(19) United States
(12) Patent Application Publication
(10) Pub. No.: US 2024/0261288 A1
(43) Pub. Date: Aug. 8, 2024

(54) COMBINATION OF A 3-(IMIDAZOL-4-YL)-4-(AMINO)-BENZENESULFONAMIDE TEAD INHIBITOR WITH AN EGFR INHIBITOR AND/OR MEK INHIBITOR FOR USE IN THE TREATMENT OF LUNG CANCER

(71) Applicant: lkena Oncology, Inc., Boston, MA (US)

(72) Inventors: Alfredo C. CASTRO, Somerville, MA (US); Michael BURKE, Melrose, MA (US); Benjamin AMIDON, Arlington, MA (US); Hyejin FROSCH, Wakefield, MA (US)

(21) Appl. No.: 18/273,411

(22) PCT Filed: Jan. 25, 2022
(86) PCT No.: PCT/US2022/070330

§ 371 Date: Jul. 20, 2023

Related U.S. Application Data
(60) Provisional application No. 63/141,105, filed on Jan. 25, 2021.

(51) Int. Cl. A61K 31/519; A61K 31/085; A61K 31/136; A61K 31/167; A61K 31/18; A61K 31/403; A61K 31/4184; A61K 31/422; A61K 31/427; A61K 31/437; A61K 31/438; A61K 31/4439; A61K 31/4468; A61K 31/454; A61K 31/4725; A61K 39/00; A61P 35/00; C07K 16/28

(52) U.S. Cl. CPC A61K 31/519; A61K 31/085; A61K 31/136; A61K 31/167; A61K 31/18; A61K 31/403; A61K 31/4184; A61K 31/422; A61K 31/427; A61K 31/437; A61K 31/438; A61K 31/4439; A61K 31/4468; A61K 31/454; A61K 31/4725; A61P 35/00; C07K 16/2863; A61K 2039/505

(57) ABSTRACT

The present invention provides TEAD inhibitors, and methods of use thereof.

### CROSS-REFERENCE TO RELATED APPLICATIONS

This application claims the benefit of and priority to under 35 U.S.C. § 119(e) of U.S. Provisional Patent Application Ser. No. 63/141,105, filed Jan. 25, 2021; the contents of which are hereby incorporated by reference in their entirety.

### TECHNICAL FIELD OF THE INVENTION

The present invention relates to TEAD inhibitors, compositions thereof, and use of a TEAD inhibitor in combination with an EGFR inhibitor and/or a MEK inhibitor for treatment of cancer.

### BACKGROUND OF THE INVENTION

Yes-associated protein (YAP) and transcriptional co-activator with PDZ-binding motif (TAZ) are transcriptional co-activators of the Hippo pathway network and regulate cell proliferation, migration, and apoptosis. Inhibition of the Hippo pathway promotes YAP/TAZ translocation to the nucleus, wherein YAP/TAZ interact with TEAD transcription factors and coactivate the expression of target genes and promote cell proliferation. Hyperactivation of YAP and TAZ and/or mutations in one or more members of the Hippo pathway network have been implicated in numerous cancers.

### SUMMARY OF THE INVENTION

It has been found that the combination of a TEAD inhibitor and an EGFR inhibitor has synergistic effects in treating cancer. Accordingly, in one aspect, the present invention provides a method for treating cancer in a patient comprising administering to the patient a therapeutically effective amount of a TEAD inhibitor and an EGFR inhibitor.

It has been also found that the combination of a TEAD inhibitor and a MEK inhibitor has synergistic effects in treating cancer. Accordingly, in one aspect, the present invention provides a method for treating cancer in a patient comprising administering to the patient a therapeutically effective amount of a TEAD inhibitor and a MEK inhibitor.

It has also been found that the combination of a TEAD inhibitor and an EGFR inhibitor has additional synergistic effects in treating cancer when used in further combination with an MEK inhibitor. Accordingly, in one aspect, the present invention provides a method for treating cancer in a patient comprising administering to the patient a therapeutically effective amount of a TEAD inhibitor, an EGFR inhibitor, and an MEK inhibitor.

In some embodiments, a TEAD inhibitor is selected from those as described herein. In some embodiments, an EGFR inhibitor is selected from those as described herein. In some embodiments, an MEK inhibitor is selected from those as described herein. In some embodiments, a cancer is selected from those as described herein.

### BRIEF DESCRIPTION OF FIGURES

FIG. 1 demonstrates the apoptosis induction effects of a TEAD inhibitor T-A-32, an EGFR inhibitor Osimertinib, an MEK inhibitor Trametinib, and combinations thereof in HCC4006 and HCC827 cells.

FIG. 2 demonstrates the effects of a TEAD inhibitor T-A-32, an EGFR inhibitor Osimertinib, an MEK inhibitor Trametinib, and combinations thereof on H1975 Tumor Growth in nude nu/nu mice.

FIG. 3 demonstrates the effects of a TEAD inhibitor T-A-32, a MEK inhibitor Trametinib, and a combination thereof on tumor growth and volume in an HCT-116 tumor model, which is a KRAS G13D mutant tumor.

FIG. 4 demonstrates the effects of a TEAD inhibitor T-A-32, a MEK inhibitor Trametinib, and a combination thereof on tumor growth and volume in an A549 tumor model, which is a KRAS G12S mutant tumor.

FIG. 5 demonstrates the effects of a TEAD inhibitor T-A-32, a MEK inhibitor Trametinib, and a combination thereof on tumor growth and volume in a LoVo tumor model, which is a KRAS G12D mutant tumor.

### DETAILED DESCRIPTION OF THE INVENTION

### 1. General Description of Certain Embodiments of the Invention

As described herein, a combination of a TEAD inhibitor and an EGFR inhibitor demonstrated unexpected synergistic effects in treating cancer. For example, a combination of a TEAD inhibitor T-A-32 and an EGFR inhibitor Osimertinib significantly reduced H1975 tumor growth in nude nu/nu mice compared to each agent alone, as shown in Examples 1 and 2. Accordingly, provided herein are methods and uses for treating cancer comprising administering a TEAD inhibitor and an EGFR inhibitor to patients in need thereof.

As also described herein, a combination of a TEAD inhibitor and an EGFR inhibitor demonstrated additional unexpected synergistic effects in treating cancer when further combined with an MEK inhibitor. For example, a combination of a TEAD inhibitor T-A-32, an EGFR inhibitor Osimertinib, and an MEK inhibitor Trametinib significantly increased apoptosis in HCC4006 and HCC827 cells and reduced H1975 tumor growth in nude nu/nu mice, compared to a combination of a TEAD inhibitor and an EGFR inhibitor Osimertinib, as shown in Examples 1 and 2. Accordingly, provided herein are methods and uses for treating cancer comprising administering a TEAD inhibitor, an EGFR inhibitor, and an MEK inhibitor to patients in need thereof.

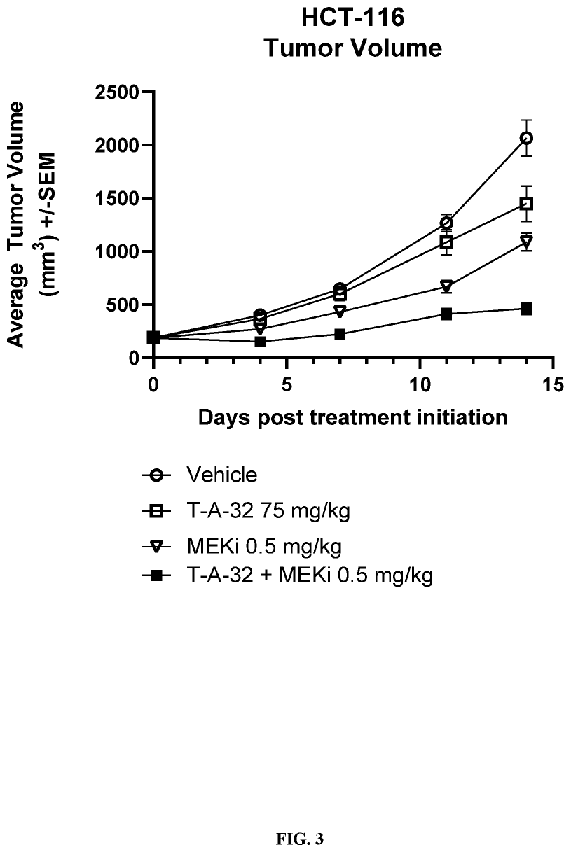

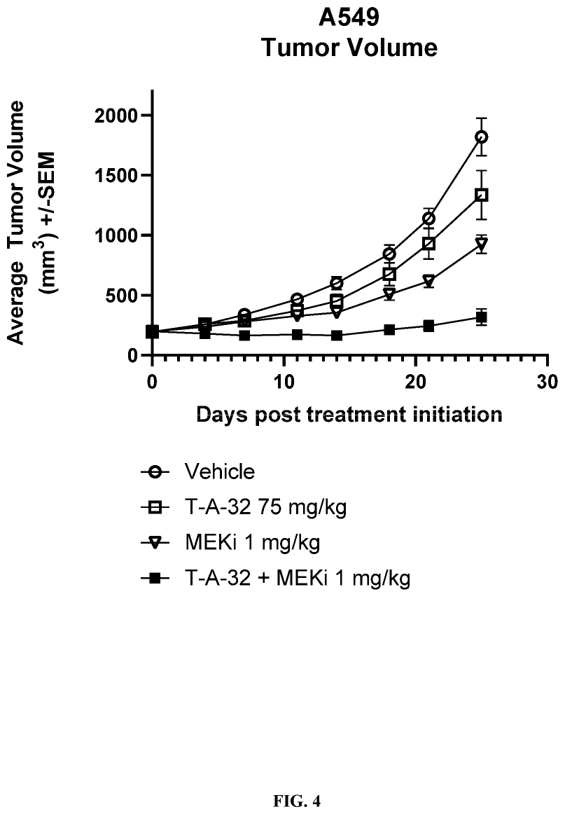

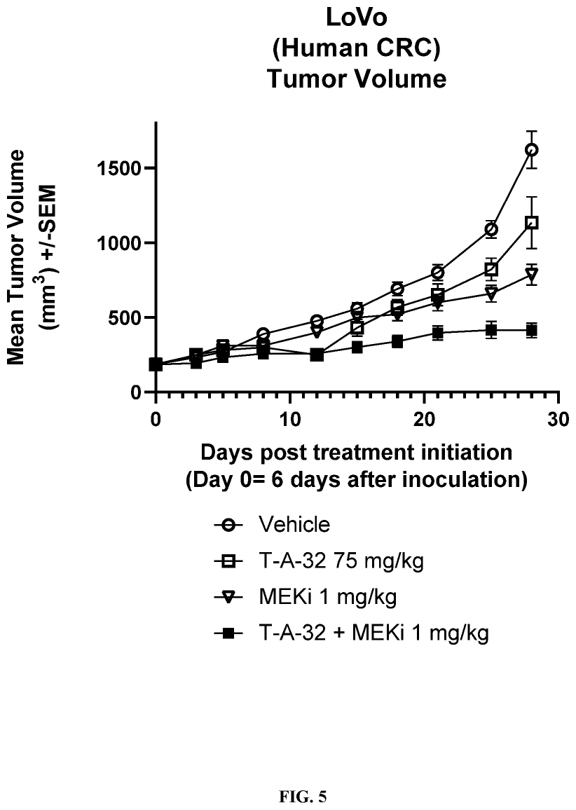

As also described herein, a combination of a TEAD inhibitor and a MEK inhibitor demonstrated additional unexpected synergistic effects in treating cancer, in various mouse xenograft models harboring KRAS mutations. For example, a combination of a TEAD inhibitor T-A-32 and a MEK inhibitor Trametinib reduced HCT-116 tumor growth, a KRAS G13D mutant human colorectal carcinoma xenograft model, in nude nu/nu mice, compared to either agent alone, as shown in Example 4 and FIG. 3. In addition, a combination of a TEAD inhibitor T-A-32 and a MEK inhibitor Trametinib reduced growth of A549 tumors, a KRAS G12S mutant tumor, in nude nu/nu mice, compared to either agent alone, as shown in Example 5 and FIG. 4. Furthermore, a combination of a TEAD inhibitor T-A-32 and a MEK inhibitor Trametinib reduced LoVo tumor growth, a KRAS G12D mutant human colorectal adenocarcinoma xenograft model, in nude nu/nu mice, compared to either agent alone, as shown in Example 6 and FIG. 5. Accordingly, provided herein are methods and uses for treating cancer comprising administering a TEAD inhibitor and an MEK inhibitor to patients in need thereof.

In one aspect, the present invention provides a method for treating cancer in a patient, comprising administering to the patient a therapeutically effective amount of a TEAD inhibitor and an EGFR inhibitor.

In one aspect, the present invention provides a method for treating cancer in a patient, comprising administering to the patient a therapeutically effective amount of a TEAD inhibitor and a MEK inhibitor.

In one aspect, the present invention provides a method for treating cancer in a patient, comprising administering to the patient a therapeutically effective amount of a TEAD inhibitor, an EGFR inhibitor, and an MEK inhibitor.

In some embodiments, a TEAD inhibitor is selected from those as described herein. In some embodiments, an EGFR inhibitor is selected from those as described herein. In some embodiments, an MEK inhibitor is selected from those as described herein. In some embodiments, a cancer is selected from those as described herein.

### 2. Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

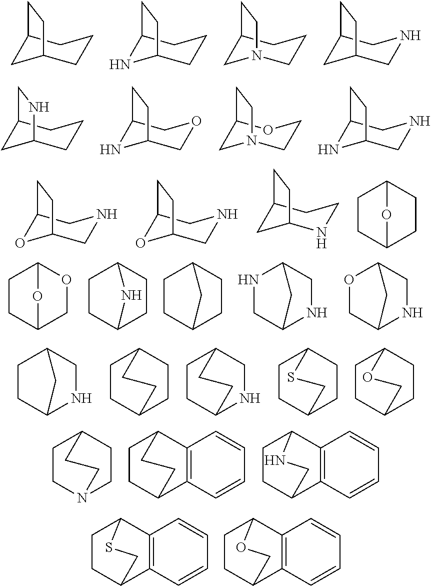

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

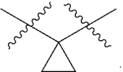 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}3; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR⁰²; —C(S)NR^{∘}₂; —C(S)SR^{∘}; —SC(S)SR^{∘}, —(CH₂)₀₋₄OC(O)NR⁰²; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{•}₂, ═NNHC(O)R^{•}, ═NNHC(O)OR^{•}, ═NNHS(O)₂R^{•}, ═NR^{•}, ═NOR^{•}, —O(C(R^{•}2))₂₋₃O—, or —S(C(R^{•}₂))₂₋₃S—, wherein each independent occurrence of R^{•} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{•}₂)₂₋₃O—, wherein each independent occurrence of R^{•} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. et al. et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium, and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the term “provided compound” refers to any TEAD inhibitor genus, subgenus, and/or species set forth herein.

As used herein, the terms “TEAD inhibitor” or “TEAD antagonist” are defined as a compound that binds to and/or inhibits TEAD with measurable affinity. In some embodiments, inhibition in the presence of a TEAD inhibitor or a TEAD antagonist is observed in a dose-dependent manner. In some embodiments, the measured signal (e.g., signaling activity or biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100% lower than the signal measured with a negative control under comparable conditions. The potency of an inhibitor is usually defined by its IC₅₀ value (half maximal inhibitory concentration or concentration required to inhibit 50% of the agonist response). The lower the IC₅₀ value the greater the potency of the antagonist and the lower the concentration that is required to inhibit the maximum biological response. In certain embodiments, an inhibitor has an IC₅₀ and/or binding constant of less than about 100 μM, less than about 50 μM, less than about 1 μM, less than about 500 nM, less than about 100 nM, less than about 10 nM, or less than about 1 nM.

The terms “measurable affinity” and “measurably inhibit,” as used herein, means a measurable change or inhibition in TEAD activity between a sample comprising a compound of the present invention, or composition thereof, and TEAD, and an equivalent sample comprising TEAD, in the absence of said compound, or composition thereof.

As used herein, an “EGFR inhibitor” refers to any inhibitor or blocker or antagonist that binds to and/or inhibits epidermal growth factor receptor (EGFR). In some embodiments, an EGFR inhibitor is selected from those as described in Ayati et al, “A review on progression of epidermal growth factor receptor (EGFR) inhibitors as an efficient approach in cancer targeted therapy,” Bioorganic Chemistry 2020, 99: 103811, the contents of which are incorporated herein by reference in their entirety. In some embodiments, an EGFR inhibitor is selected from cetuximab, necitumumab, panitumumab, zalutumumab, nimotuzumab, and matuzumab. In some embodiments, an EGFR inhibitor is cetuximab. In some embodiments, an EGFR inhibitor is necitumumab. In some embodiments, an EGFR inhibitor is panitumumab. In some embodiments, an EGFR inhibitor is zalutumumab. In some embodiments, an EGFR inhibitor is nimotuzumab. In some embodiments, an EGFR inhibitor is matuzumab.

In some embodiments, an EGFR inhibitor is selected from osimertinib, gefitinib, erlotinib, lapatinib, neratinib, vandetanib, afatinib, brigatinib, dacomitinib, and icotinib. In some embodiments, an EGFR inhibitor is osimertinib. In some embodiments, an EGFR inhibitor is gefitinib. In some embodiments, an EGFR inhibitor is erlotinib. In some embodiments, an EGFR inhibitor is lapatinib. In some embodiments, an EGFR inhibitor is neratinib. In some embodiments, an EGFR inhibitor is vandetanib. In some embodiments, an EGFR inhibitor is afatinib. In some embodiments, an EGFR inhibitor is brigatinib. In some embodiments, an EGFR inhibitor is dacomitinib. In some embodiments, an EGFR inhibitor is icotinib.

In some embodiments, an EGFR inhibitor is a “1st generation EGFR tyrosine kinase inhibitor” (“1st generation TKI”). A 1^{st} generation TKI refers to reversible EGFR inhibitors, such as gefitinib and erlotinib, which are effective in first-line treatment of, for example, NSCLC harboring EGFR activating mutations, such as deletions in exon 19 and exon 21 L858R mutation.

In some embodiments, an EGFR inhibitor is a “2nd generation EGFR tyrosine kinase inhibitor” (“2nd generation TKI”). A 2^{nd} generation TKI refers to covalent irreversible EGFR inhibitors, such as afatinib and dacomitib, which are effective in first-line treatment of NSCLC harboring EGFR activating mutations, such as deletions in exon 19 and exon 21 L858R mutation.

In some embodiments, an EGFR inhibitor is a “3rd generation EGFR tyrosine kinase inhibitor” (“3rd generation TKI”). A 3rd generation TKI refers to covalent irreversible EGFR inhibitors, such as osimertinib and lazertinib, which are selective to the EGFR activating mutations, such as deletions in exon 19 and exon 21 L858R, alone or in combination with T790M mutation, and have lower inhibitory activity against wild-type EGFR.

As used herein, a “MEK inhibitor” refers to any inhibitor or blocker or antagonist that binds to and/or inhibits mitogen-activated protein kinase enzymes MEK1 and/or MEK2. In some embodiments, a MEK inhibitor is selected from those as described in Cheng et al, “Current Development Status of MEK Inhibitors,” Molecules 2017, 22, 1551, the contents of which are incorporated herein by reference in their entirety. In certain embodiments, a MEK inhibitor is selected from binimetinib (MEK162, ARRY-438162, ARRAY BIOPHARMA INC.), cobimetinib (COTELLIC®, Exelexis/Genentech/Roche), refametinib (BAY 86-9766, RDEA119; Bayer AG), selumetinib (AZD6244, ARRY-142886; ASTRAZENECA), trametinib (MEKINIST®, Novartis), mirdametinib (PD-0325901, Spring Works Therapeutics), pimasertib (AS703026, MSC1936369B, Merck KGaA) or a pharmaceutically acceptable salt and/or solvate of any of the foregoing. In certain embodiments, a MEK inhibitor is binimetinib, cobimetinib, selumetinib, trametinib, mirdametinib, pimasertib, or a pharmaceutically acceptable salt and/or solvate of any of the foregoing. Other examples of MEK inhibitors for use in the methods and uses described herein include, but are not limited to, E6201 (Eisai Co Ltd./Strategia Therapeutics), GDC-0623 (RG 7421, Genentech, Inc.), CH5126766 (RO5126766, Chugai 232Pharmaceutical Co., Roche), HL-085 (Shanghai Kechow Pharma, Inc.), SHR7390 (HENGRUI MEDICINE), TQ-B3234 (CHIATAI TIANQING), CS-3006 (CSTONE Pharmaceuticals), FCN-159 (FosunPharmaceuticals), VS-6766 (Verastem Oncology), and IMM-1-104 (Immuneering Corp.). Other examples of MEK inhibitors in the methods and uses described herein include, but are not limited to, those described in international patent publications WO2005/121142, WO2014/169843, WO2016/035008, WO2016/168704, WO2020/125747, WO2021/142144, WO2021/142345, and WO2021/149776, the contents of each of which are herein incorporated by reference in their entireties.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

### 3. Description of Exemplary Methods and Uses

In some aspects and embodiments, the present invention provides a method for treating cancer in a patient comprising administering to the patient a therapeutically effective amount of a TEAD inhibitor and an EGFR inhibitor.

In some aspects and embodiments, the present invention provides a method for treating cancer in a patient comprising administering to the patient a therapeutically effective amount of a TEAD inhibitor, an EGFR inhibitor, and an MEK inhibitor.

In some aspects and embodiments, the present invention provides a use of a TEAD inhibitor for the treatment of cancer in combination with an EGFR inhibitor. In some aspects and embodiments, the present invention provides a use of a TEAD inhibitor for the treatment of cancer in combination with an EGFR inhibitor and an MEK inhibitor. In some embodiments, the present invention provides a use of a TEAD inhibitor in the manufacture of a medicament for the treatment of cancer, wherein the medicament is for use in combination with an EGFR inhibitor. In some embodiments, the present invention provides a use of a TEAD inhibitor in the manufacture of a medicament for the treatment of cancer, wherein the medicament is for use in combination with an EGFR inhibitor and an MEK inhibitor. In some embodiments, a medicament comprises a TEAD inhibitor, or a pharmaceutical composition thereof. In some embodiments, a pharmaceutical composition comprising a TEAD inhibitor is as described herein.

In some aspects and embodiments, the present invention provides a use of a TEAD inhibitor for the treatment of cancer in combination with a MEK inhibitor. In some embodiments, the present invention provides a use of a TEAD inhibitor in the manufacture of a medicament for the treatment of cancer, wherein the medicament is for use in combination with a MEK inhibitor. In some embodiments, a medicament comprises a TEAD inhibitor, or a pharmaceutical composition thereof. In some embodiments, a pharmaceutical composition comprising a TEAD inhibitor is as described herein.

In some embodiments, a cancer is selected from those as described herein. In some embodiments, a cancer is an EGFR mutant resistant cancer. In some embodiments, a cancer is a lung cancer. In some embodiments, a cancer is an EGFR mutant resistant lung cancer. In some embodiments, a cancer is a non-small cell lung cancer (NSCLC). In some embodiments, a cancer is an EGFR mutant resistant NSCLC.

In some embodiments, a TEAD inhibitor is a compound capable of binding to one or more of TEAD1, TEAD2, TEAD3, or TEAD4.

In some embodiments, a TEAD inhibitor is a compound capable of binding to TEAD1. In some embodiments, a TEAD inhibitor is a compound capable of binding to TEAD2. In some embodiments, a TEAD inhibitor is a compound capable of binding to TEAD3. In some embodiments, a TEAD inhibitor is a compound capable of binding to TEAD4.

In some embodiments, a TEAD inhibitor is a compound as described in Pobbati et al., “Targeting the Central Pocket in Human Transcription Factor TEAD as a Potential Cancer Therapeutic Strategy,” Structure 2015, 23, 2076-2086; Gibault et al., “Targeting Transcriptional Enhanced Associate Domains (TEADs),” J. Med. Chem. 2018, 61, 5057-5072; Bum-Erdene et al., “Small-Molecule Covalent Modification of Conserved Cysteine Leads to Allosteric Inhibition of the TEAD·Yap Protein-Protein Interaction,” Cell Chemical Biology 2019, 26, 1-12; Holden et. al., “Small Molecule Dysregulation of TEAD Lipidation Induces a Dominant-Negative Inhibition of HippoPathway Signaling,” Cell Reports 2020, 31, 107809; WO 2017/053706, WO 2017/111076, WO 2018/204532, WO 2018/235926, US 20190010136, WO 2019/040380, WO 2019/113236, WO 2019/222431, WO 2019/232216, WO 2020/051099, WO 2020/081572, WO 2020/097389, WO 2020/190774, or WO 2020/214734, the contents of each of which are herein incorporated by reference in their entirety.

In some embodiments, a TEAD inhibitor is selected from the compounds as described herein. In some embodiments, a TEAD inhibitor is administered at about 1 mg/kg to about 100 mg/kg of subject body weight per day, one or more times a day. In some embodiments, a TEAD inhibitor is administered at about 1 mg/kg to about 10 mg/kg, or about 10 mg/kg to about 25 mg/kg, or about 25 mg/kg to about 50 mg/kg, or about 50 mg/kg to about 75 mg/kg, or about 75 mg/kg to about 100 mg/kg of subject body weight per day, one or more times a day. In some embodiments, a TEAD inhibitor is administered at about 2.5 mg/kg to about 90 mg/kg, or about 5 mg/kg to about 80 mg/kg, or about 7.5 mg/kg to about 70 mg/kg, or about 10 mg/kg to about 50 mg/kg, or about 12.5 mg/kg to about 40 mg/kg, or about 15 mg/kg to about 30 mg/kg of subject body weight per day, one or more times a day. In some embodiments, a TEAD inhibitor is administered at about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 50 mg/kg, about 60 mg/kg, about 70 mg/kg, about 75 mg/kg, about 80 mg/kg, or about 85 mg/kg of subject body weight per day, one or more times a day.

In some embodiments, an EGFR inhibitor is selected from cetuximab, necitumumab, panitumumab, zalutumumab, nimotuzumab, and matuzumab. In some embodiments, an EGFR inhibitor is cetuximab. In some embodiments, an EGFR inhibitor is necitumumab. In some embodiments, an EGFR inhibitor is panitumumab. In some embodiments, an EGFR inhibitor is zalutumumab. In some embodiments, an EGFR inhibitor is nimotuzumab. In some embodiments, an EGFR inhibitor is matuzumab.

In some embodiments, an EGFR inhibitor is selected from osimertinib, gefitinib, erlotinib, lapatinib, neratinib, vandetanib, afatinib, brigatinib, dacomitinib, and icotinib. In some embodiments, an EGFR inhibitor is Osimertinib. In some embodiments, an EGFR inhibitor is gefitinib. In some embodiments, an EGFR inhibitor is erlotinib. In some embodiments, an EGFR inhibitor is lapatinib. In some embodiments, an EGFR inhibitor is neratinib. In some embodiments, an EGFR inhibitor is vandetanib. In some embodiments, an EGFR inhibitor is afatinib. In some embodiments, an EGFR inhibitor is brigatinib. In some embodiments, an EGFR inhibitor is dacomitinib. In some embodiments, an EGFR inhibitor is icotinib.

In some embodiments, an EGFR inhibitor is a “1st generation EGFR tyrosine kinase inhibitor” (1st generation TKI). A 1^{st} generation TKI refers to reversible EGFR inhibitors, such as gefitinib and erlotinib, which are effective in first-line treatment of NSCLC harboring EGFR activating mutations such as deletions in exon 19 and exon 21 L858R mutation.

In some embodiments, an EGFR inhibitor is a “2nd generation EGFR tyrosine kinase inhibitor” (2nd generation TKI). A 2^{nd} generation TKI refers to covalent irreversible EGFR inhibitors, such as afatinib and dacomitib, which are effective in first-line treatment of NSCLC harboring EGFR activating mutations such as deletions in exon 19 and exon 21 L858R mutation.

In some embodiments, an EGFR inhibitor is a “3rd generation EGFR tyrosine kinase inhibitor” (3rd generation TKI). A 3rd generation TKI refers to covalent irreversible EGFR inhibitors, such as osimertinib and lazertinib, which are selective to the EGFR activating mutations, such as deletions in exon 19 and exon 21 L858R, alone or in combination with T790M mutation, and have lower inhibitory activity against wild-type EGFR.

In some embodiments, a MEK inhibitor is selected from refametinib, selumetinib, trametinib, and cobimetinib. In some embodiments, a MEK inhibitor is refametinib. In some embodiments, a MEK inhibitor is selumetinib. In some embodiments, a MEK inhibitor is trametinib. In some embodiments, a MEK inhibitor is cobimetinib.

In some embodiments, a MEK inhibitor is selected from:

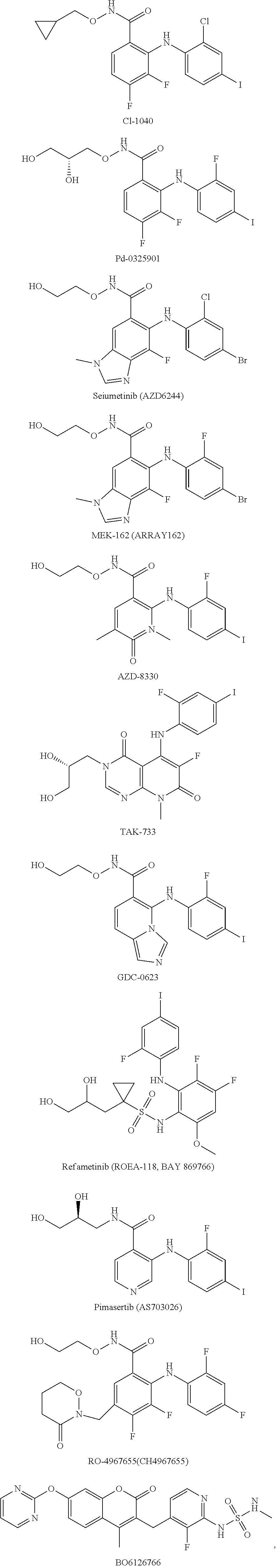

or a pharmaceutically acceptable salt thereof.

In some embodiments, a MEK inhibitor is WX-554. WX-554 is a selective, noncompetitive MEK1/2 inhibitor, which has been tested in dose-escalation phase I/II studies (ClinicalTrials.gov: NCT01859351, NCT01581060).

In some embodiments, a MEK inhibitor is HL-085. HL-085 is an orally active, selective MEK inhibitor, which has been tested in phase I clinical study.

In some embodiments, a MEK inhibitor is selected from:

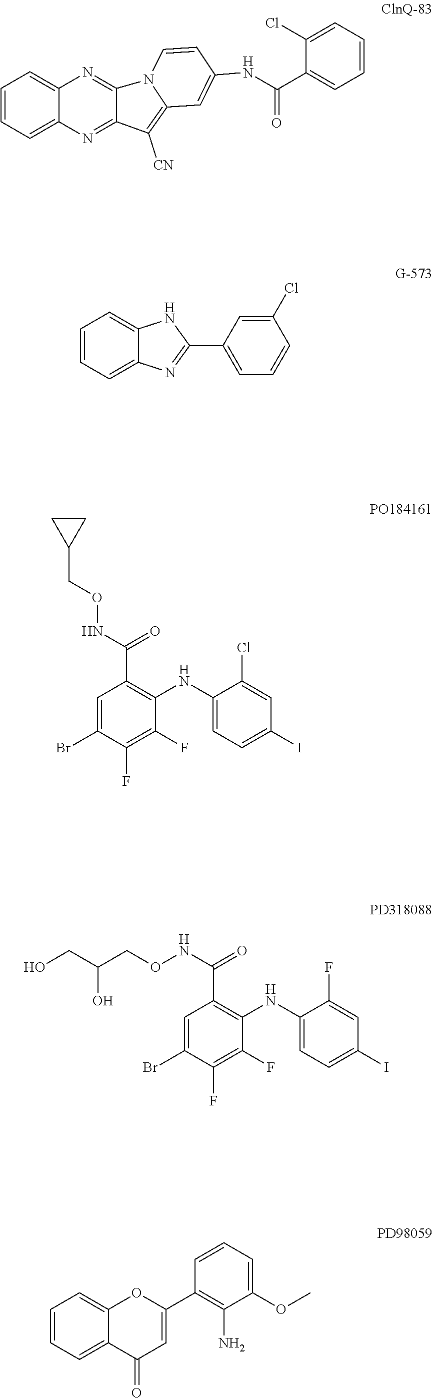 [SMILES: N#Cc1c2nc3ccccc3nc2n2ccc(NC(=O)c3ccccc3Cl)cc12 ; Clc1cccc(-c2nc3ccccc3[nH]2)c1 ; O=C(NOCC1CC1)c1cc(Br)c(F)c(F)c1Nc1ccc(I)cc1Cl ; O=C(NOCC(O)CO)c1cc(Br)c(F)c(F)c1Nc1ccc(I)cc1F ; COc1cccc(-c2cc(=O)c3ccccc3o2)c1N]

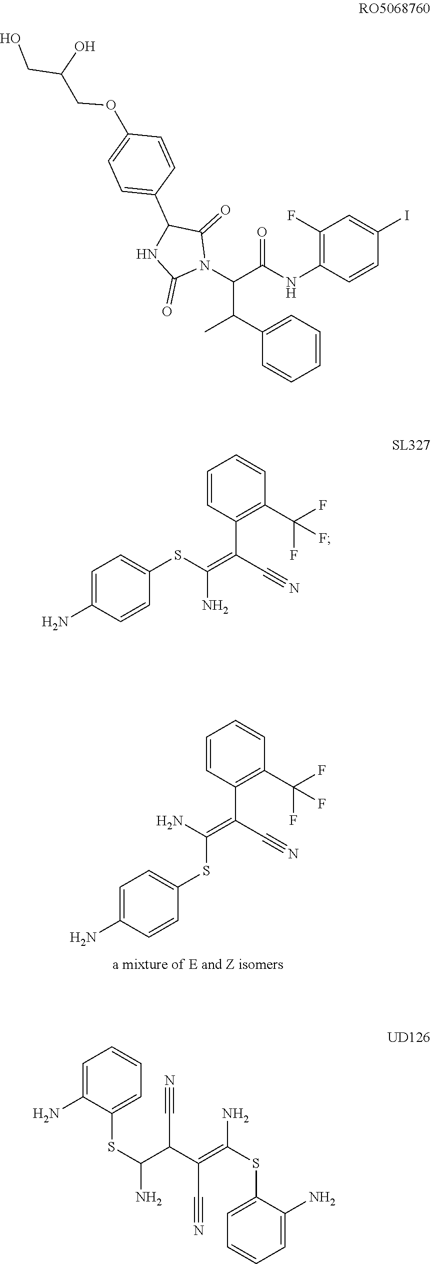 [SMILES: CC(c1ccccc1)C(C(=O)Nc1ccc(I)cc1F)N1C(=O)NC(c2ccc(OCC(O)CO)cc2)C1=O ; N#C/C(=C(\N)Sc1ccc(N)cc1)c1ccccc1C(F)(F)F ; N#C/C(=C(/N)Sc1ccc(N)cc1)c1ccccc1C(F)(F)F ; N#C/C(=C(/N)Sc1ccccc1N)C(C#N)C(N)Sc1ccccc1N]

or a pharmaceutically acceptable salt thereof.

### 1. TEAD Inhibitors of Formulae A, and A-1 to A-50

In certain embodiments, a TEAD inhibitor is selected from those as described in WO 2020/243415, the contents of which are herein incorporated by reference in their entirety.

In certain embodiments, a TEAD inhibitor is a compound of Formula A

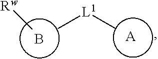

or a pharmaceutically acceptable salt thereof, wherein
L¹ is C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—;
Ring A is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur;
Ring B is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, a 8-10 membered bicyclic heteroaromatic ring having 1-5 heteroatoms independently selected from nitrogen, oxygen, or sulfur;
R^{w} is an optionally substituted 4-, 5-, or 6-membered ring having 1, 2, 3, or 4 heteroatoms independently selected from nitrogen, oxygen, or sulfur; and
each R is independently —H or optionally substituted —C₁₋₆ aliphatic.

In certain embodiments, a TEAD inhibitor is a compound of Formula A-1:

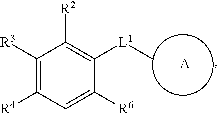

or a pharmaceutically acceptable salt thereof, wherein
L¹ is C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—;
Ring A is a 4-, 5-, or 6-membered saturated or partially unsaturated monocyclic carbocyclic ring, phenyl, a 4-, 5-, or 6-membered saturated or partially unsaturated monocyclic heterocyclic ring having 1-2 heteroatoms independently selected from nitrogen, oxygen, or sulfur, or a 5-6 membered monocyclic heteroaromatic ring having 1, 2, 3, or 4 heteroatoms independently selected from nitrogen, oxygen, or sulfur, wherein Ring A is optionally substituted 1-2 times by -halogen, —CN, —NO₂, or —C₁₋₆ aliphatic substituted 0-6 times by -halogen, —CN, or —NO₂;
R² is —H, or an optionally substituted 4-, 5-, or 6-membered ring having 1, 2, 3, or 4 heteroatoms independently selected from nitrogen, oxygen, or sulfur;
R³ is —H;
R⁴ is —H, halogen, —S(O)₂N(R)₂, —S(O)N(R)₂, or —C(O)N(R)₂;
R⁶ is —H or —C₁₋₆ aliphatic substituted 0-6 times by -halogen, —CN, or —NO₂; and
each R is independently —H or optionally substituted —C₁₋₆ aliphatic.

As defined generally above, L¹ is C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is a covalent bond, or a C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is a covalent bond.

In some embodiments, L¹ is C₁₋₆ bivalent straight or branched hydrocarbon chain wherein 1, 2, or 3 methylene units of the chain are independently and optionally replaced with —O—, —CH(OR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, or —N(R)C(O)N(R)—.

In some embodiments, L¹ is C₁₋₆ bivalent straight or branched hydrocarbon chain wherein 1, 2, or 3 methylene units of the chain are optionally replaced with —CH(SR)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is C₁₋₆ bivalent straight or branched hydrocarbon chain wherein 1, 2, or 3 methylene units of the chain are independently and optionally replaced with —O—, —S—, or —N(R)—.

In some embodiments, L¹ is C₁₋₆ bivalent straight or branched hydrocarbon chain wherein 1, 2, or 3 methylene units of the chain are independently and optionally replaced with —CH(OR)—, —CH(SR)—, or —CH(N(R)₂)—.

In some embodiments, L¹ is C₁₋₆ bivalent straight or branched hydrocarbon chain wherein 1, 2, or 3 methylene units of the chain are independently and optionally replaced with —C(O)—, —C(O)O—, —OC(O)—, —SO—, —SO₂—, —C(S)—, —C(S)O—, or —OC(S)—.

In some embodiments, L¹ is C₁₋₆ bivalent straight or branched hydrocarbon chain wherein 1, 2, or 3 methylene units of the chain are independently and optionally replaced with —C(O)N(R)—, —(R)NC(O)—, —OC(O)N(R)—, —(R)NC(O)O—, —N(R)C(O)N(R)—, —SO₂N(R)—, —(R)NSO₂—, —C(S)N(R)—, —(R)NC(S)—, or —(R)NC(S)N(R)—.

In some embodiments, L¹ is —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is —O—, —CH(OR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, or —N(R)C(O)N(R)—.

In some embodiments, L¹ is —CH(SR)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is —O—, —S—, or —N(R)—. In some embodiments, L¹ is —O—. In some embodiments, L¹ is —S—. In some embodiments, L¹ is —N(R)—. In some embodiments, L¹ is —NH—.

In some embodiments, L¹ is —CH(OR)—, —CH(SR)—, or —CH(N(R)₂)—. In some embodiments, L¹ is —CH(OR)—. In some embodiments, L¹ is —CH(SR)—. In some embodiments, L¹ is —CH(N(R)₂)—.

In some embodiments, L¹ is —C(O)—, —C(O)O—, —OC(O)—, —SO—, —SO₂—, —C(S)—, —C(S)O—, or —OC(S)—. In some embodiments, L¹ is —C(O)—. In some embodiments, L¹ is —C(O)O—. In some embodiments, L¹ is —OC(O)—. In some embodiments, L¹ is —SO—. In some embodiments, L¹ is —SO₂—. In some embodiments, L¹ is —C(S)—. In some embodiments, L¹ is —C(S)O—. In some embodiments, L¹ is —OC(S)—.

In some embodiments, L¹ is —C(O)N(R)—, —(R)NC(O)—, —OC(O)N(R)—, —(R)NC(O)O—, —N(R)C(O)N(R)—, —SO₂N(R)—, —(R)NSO₂—, —C(S)N(R)—, —(R)NC(S)—, or —(R)NC(S)N(R)—. In some embodiments, L¹ is —C(O)N(R)—. In some embodiments, L¹ is —(R)NC(O)—. In some embodiments, L¹ is —OC(O)N(R)—. In some embodiments, L¹ is —(R)NC(O)O—. In some embodiments, L¹ is —N(R)C(O)N(R)—. In some embodiments, L¹ is —SO₂N(R)—. In some embodiments, L¹ is —(R)NSO₂—. In some embodiments, L¹ is —C(S)N(R)—. In some embodiments, L¹ is —(R)NC(S)—. or In some embodiments, L¹ is —(R)NC(S)N(R)—.

In some embodiments, L¹ is —CH₂—, —CH(CH₃)—, —NH—CH₂—, —NH—CH(CH₃)—, —C(O)—NH—, or —N(CH₃)—.

In some embodiments, L¹ is

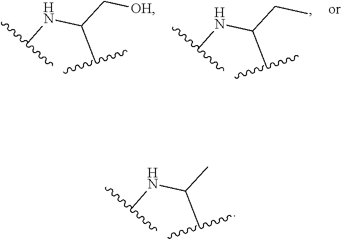

In some embodiments, L¹ is selected from those depicted in Table A, below.

As defined generally above, Ring A is a 4-, 5-, or 6-membered saturated or partially unsaturated monocyclic carbocyclic ring, phenyl, a 4-, 5-, or 6-membered saturated or partially unsaturated monocyclic heterocyclic ring having 1-2 heteroatoms independently selected from nitrogen, oxygen, or sulfur, or a 5-6 membered monocyclic heteroaromatic ring having 1, 2, 3, or 4 heteroatoms independently selected from nitrogen, oxygen, or sulfur, wherein Ring A is optionally substituted 1-2 times by halogen, —CN, —NO₂, or —C₁₋₆ aliphatic substituted 0-6 times by halogen, —CN, or —NO₂.

In some embodiments, Ring A is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur.

In some embodiments, Ring A is optionally substituted phenyl. In some embodiments, Ring A is optionally substituted 4-, 5-, or 6-membered saturated or partially unsaturated monocyclic carbocyclic ring. In some embodiments, Ring A is optionally substituted 4-, 5-, or 6-membered saturated or partially unsaturated monocyclic heterocyclic ring having 1-2 heteroatoms independently selected from nitrogen, oxygen, or sulfur. In some embodiments, Ring A is optionally substituted 5-6 membered monocyclic heteroaromatic ring having 1, 2, 3, or 4 heteroatoms independently selected from nitrogen, oxygen, or sulfur. In some embodiments, Ring A is optionally substituted 8-10 membered bicyclic aromatic ring. In some embodiments, Ring A is optionally substituted 8-10 membered bicyclic heteroaromatic ring having 1-5 heteroatoms independently selected from nitrogen, oxygen, or sulfur.

In some embodiments, Ring A is optionally substituted phenyl, a 6-membered monocyclic heteroaromatic ring having 1 or 2 nitrogen, or a 10-membered bicyclic heteroaromatic ring having 1-2 nitrogen.

In some embodiments, Ring A is optionally substituted

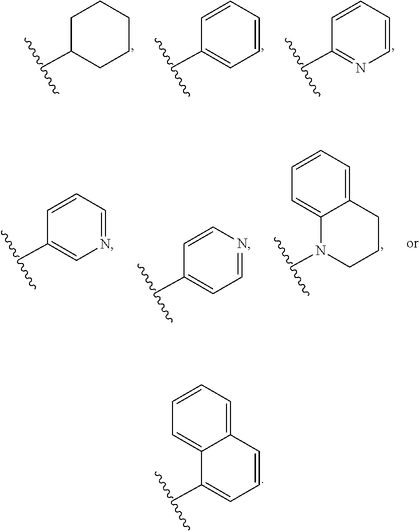

In some embodiments, Ring A is optionally substituted 1-2 times by -halogen, —CN, —NO₂, —C₁₋₆ aliphatic, or —O—C₁₋₆ aliphatic, wherein each of —C₁₋₆ aliphatic and —O—C₁₋₆ aliphatic is independently substituted 0-6 times by -halogen, —CN, or —NO₂. In some embodiments, Ring A is optionally substituted 1-2 times by halogen, —CN, —NO₂, —C₁₋₆ aliphatic, or —O—C₁₋₆ aliphatic, wherein each of —C₁₋₆ aliphatic and —O—C₁₋₆ aliphatic is independently substituted 0, 1, 2, 3, 4, 5, or 6 times by halogen, —CN, or —NO₂. In some embodiments, Ring A is optionally substituted 1-2 times by halogen, —C₁₋₆ aliphatic, or —O—C₁₋₆ aliphatic, wherein each of —C₁₋₆ aliphatic and —O—C₁₋₆ aliphatic is independently substituted 1, 2, 3, 4, 5, or 6 times by halogen.

In some embodiments, Ring A is a 4-, 5-, or 6-membered saturated or partially unsaturated monocyclic carbocyclic ring. In some embodiments, Ring A is cyclohexyl. In some embodiments, Ring A is phenyl. In some embodiments, Ring A is a 4-, 5-, or 6-membered saturated or partially unsaturated monocyclic heterocyclic ring having 1-2 heteroatoms independently selected from nitrogen, oxygen, or sulfur. In some embodiments, Ring A is a 5-6 membered monocyclic heteroaromatic ring having 1, 2, 3, or 4 heteroatoms independently selected from nitrogen, oxygen, or sulfur.

In some embodiments, Ring A is a 8-10 membered bicyclic aromatic ring. In some embodiments, Ring A is a 8-10 membered bicyclic heteroaromatic ring having 1-5 heteroatoms independently selected from nitrogen, oxygen, or sulfur.

In some embodiments, Ring A is optionally substituted 1-2 times by halogen, —CN, —NO₂, or —C₁₋₆ aliphatic substituted 0, 1, 2, 3, 4, 5, or 6 times by halogen, —CN, or —NO₂. In some embodiments, Ring A is optionally substituted 1-2 times by halogen, or —C₁₋₆ aliphatic substituted 0, 1, 2, 3, 4, 5, or 6 times by halogen.

In some embodiments, Ring A is selected from

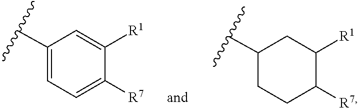

wherein each of R¹ and R⁷ is independently as described herein.

In some embodiments, Ring A is selected from

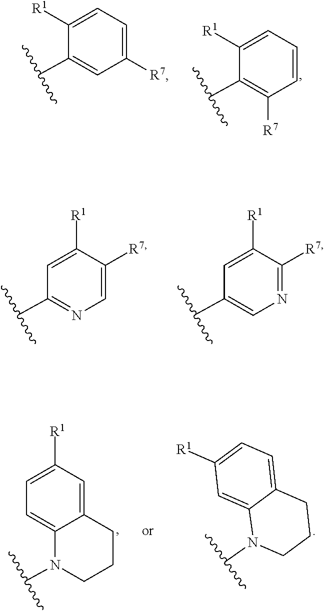

In some embodiments, R¹ is —H, -halogen, —CN, —NO₂, —C₁₋₆ aliphatic, or —O—C₁₋₆ aliphatic, wherein each of —C₁₋₆ aliphatic and —O—C₁₋₆ aliphatic is substituted 0, 1, 2, 3, 4, 5, or 6 times by -halogen, —CN, or —NO₂. In some embodiments, R¹ is unsubstituted —O—C₁₋₆ aliphatic. In some embodiments, R¹ is —OCH₃. In some embodiments, R¹ is —O—C₁₋₆ aliphatic substituted 1, 2, 3, 4, 5, or 6 times by -halogen. In some embodiments, R¹ is —O—C₁₋₃ aliphatic substituted 1, 2, 3, 4, 5, or 6 times by -halogen. In some embodiments, R¹ is —O—C₁₋₆ aliphatic substituted 1, 2, 3, 4, 5, or 6 times by —F. In some embodiments, R¹ is —OCF₃. In some embodiments, R¹ is

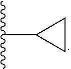 [SMILES: *C1CC1]

In some embodiments, R¹ is —H, -halogen, —CN, —NO₂, or —C₁₋₆ aliphatic substituted 0, 1, 2, 3, 4, 5, or 6 times by -halogen, —CN, or —NO₂. In some embodiments, R¹ is —H. In some embodiments, R¹ is -halogen. In some embodiments, R¹ is —F. In some embodiments, R¹ is —Cl. In some embodiments, R¹ is —Br. In some embodiments, R¹ is —CN. In some embodiments, R¹ is —NO₂. In some embodiments, R¹ is unsubstituted —C₁₋₆ aliphatic. In some embodiments, R¹ is —CH₃. In some embodiments, R¹ is —C₁₋₆ aliphatic substituted 1, 2, 3, 4, 5, or 6 times by -halogen. In some embodiments, R¹ is —C₁₋₃ aliphatic substituted 1, 2, 3, 4, 5, or 6 times by -halogen. In some embodiments, R¹ is —C₁₋₆ aliphatic substituted 1, 2, 3, 4, 5, or 6 times by —F. In some embodiments, R¹ is —C₁₋₃ aliphatic substituted 1, 2, 3, 4, 5, or 6 times by —F. In some embodiments, R¹ is —CF₃. In some embodiments, R¹ is —C₁₋₆ aliphatic substituted 1, 2, 3, 4, 5, or 6 times by —CN. In some embodiments, R¹ is —C₁₋₆ aliphatic substituted 1, 2, 3, 4, 5, or 6 times by —NO₂.

In some embodiments, R⁷ is —H, -halogen, —CN, —NO₂, —C₁₋₆ aliphatic, or —O—C₁₋₆ aliphatic, wherein each of —C₁₋₆ aliphatic and —O—C₁₋₆ aliphatic is substituted 0, 1, 2, 3, 4, 5, or 6 times by -halogen, —CN, or —NO₂. In some embodiments, R⁷ is unsubstituted —O—C₁₋₆ aliphatic. In some embodiments, R⁷ is —OCH₃. In some embodiments, R⁷ is —O—C₁₋₆ aliphatic substituted 1, 2, 3, 4, 5, or 6 times by -halogen. In some embodiments, R⁷ is —O—C₁₋₃ aliphatic substituted 1, 2, 3, 4, 5, or 6 times by -halogen. In some embodiments, R⁷ is —O—C₁₋₆ aliphatic substituted 1, 2, 3, 4, 5, or 6 times by —F. In some embodiments, R⁷ is —OCF₃. In some embodiments, R⁷ is

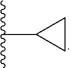 [SMILES: *C1CC1]

In some embodiments, R⁷ is —H, -halogen, —CN, —NO₂, or —C₁₋₆ aliphatic substituted 0, 1, 2, 3, 4, 5, or 6 times by -halogen, —CN, or —NO₂. In some embodiments, R⁷ is —H. In some embodiments, R⁷ is -halogen. In some embodiments, R⁷ is —F. In some embodiments, R⁷ is —Cl. In some embodiments, R⁷ is —Br. In some embodiments, R⁷ is —CN. In some embodiments, R⁷ is —NO₂. In some embodiments, R⁷ is unsubstituted —C₁₋₆ aliphatic. In some embodiments, R¹ is —CH₃. In some embodiments, R⁷ is —C₁₋₆ aliphatic substituted 1, 2, 3, 4, 5, or 6 times by -halogen. In some embodiments, R⁷ is —C₁₋₃ aliphatic substituted 1, 2, 3, 4, 5, or 6 times by -halogen. In some embodiments, R⁷ is —C₁₋₆ aliphatic substituted 1, 2, 3, 4, 5, or 6 times by —F. In some embodiments, R⁷ is —C₁₋₃ aliphatic substituted 1, 2, 3, 4, 5, or 6 times by —F. In some embodiments, R⁷ is —CF₃. In some embodiments, R⁷ is —C₁₋₆ aliphatic substituted 1, 2, 3, 4, 5, or 6 times by —CN. In some embodiments, R⁷ is —C₁₋₆ aliphatic substituted 1, 2, 3, 4, 5, or 6 times by —NO₂.

In some embodiments, Ring A is

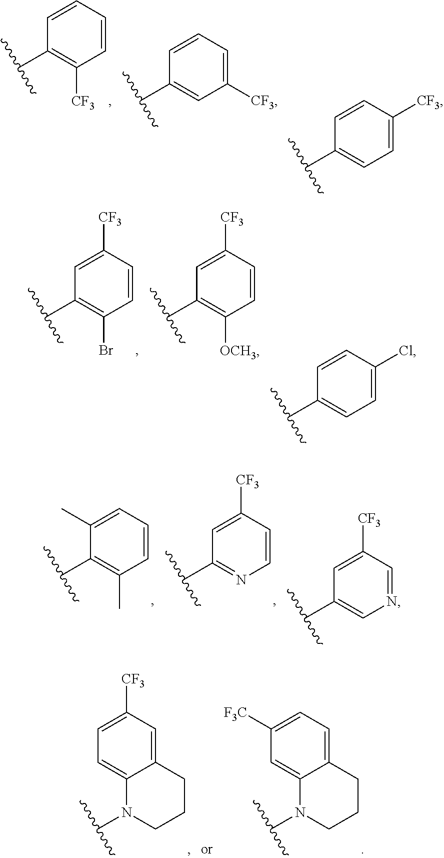

In some embodiments, Ring A is

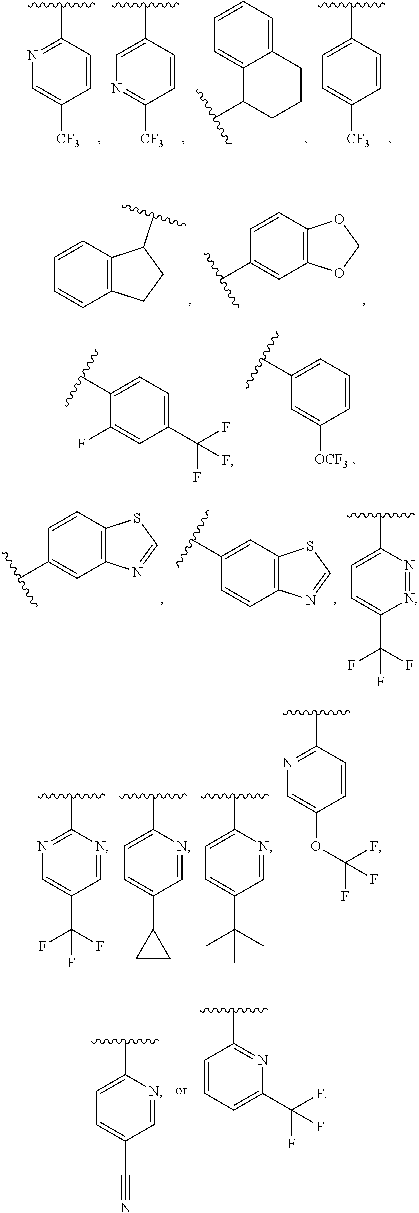

In some embodiments, Ring A is selected from those depicted in Table A, below.

As defined generally above, Ring B is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, a 8-10 membered bicyclic heteroaromatic ring having 1-5 heteroatoms independently selected from nitrogen, oxygen, or sulfur.

In some embodiments, Ring B is optionally substituted phenyl. In some embodiments, Ring B is optionally substituted 4-, 5-, or 6-membered saturated or partially unsaturated monocyclic carbocyclic ring. In some embodiments, Ring B is optionally substituted 4-, 5-, or 6-membered saturated or partially unsaturated monocyclic heterocyclic ring having 1-2 heteroatoms independently selected from nitrogen, oxygen, or sulfur. In some embodiments, Ring B is optionally substituted 5-6 membered monocyclic heteroaromatic ring having 1, 2, 3, or 4 heteroatoms independently selected from nitrogen, oxygen, or sulfur. In some embodiments, Ring B is optionally substituted 8-10 membered bicyclic aromatic ring. In some embodiments, Ring B is optionally substituted 8-10 membered bicyclic heteroaromatic ring having 1-5 heteroatoms independently selected from nitrogen, oxygen, or sulfur.

In some embodiments, Ring B is optionally substituted phenyl or a 6-membered monocyclic heteroaromatic ring having 1 or 2 nitrogen.

In some embodiments, Ring B is optionally substituted

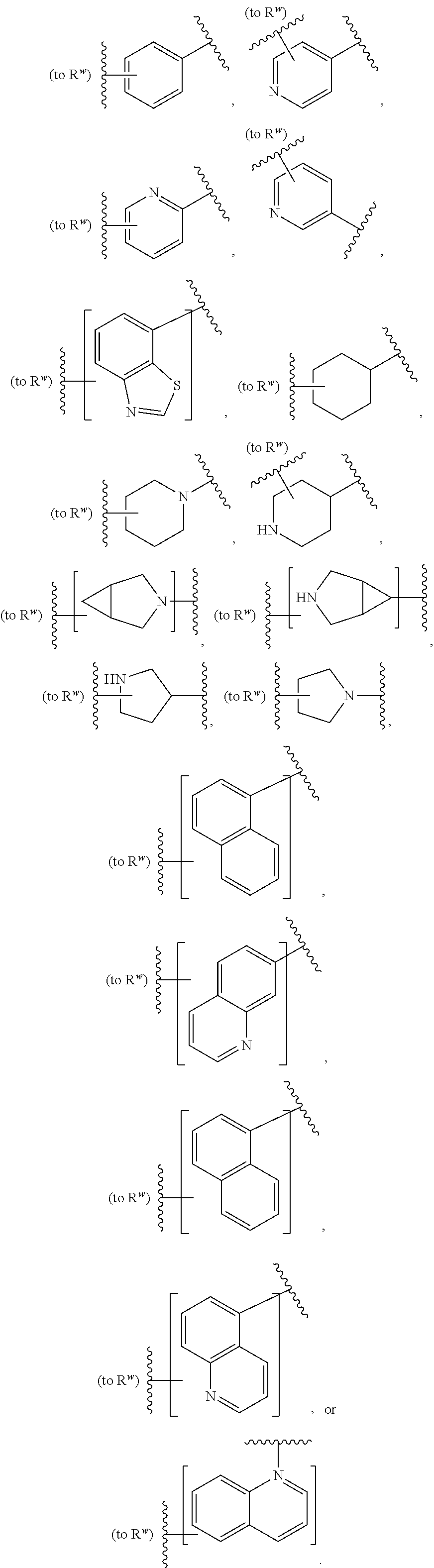

In some embodiments, Ring B is optionally substituted 1-4 times by halogen, —S(O)₂N(R)₂, —S(O)N(R)₂, —C(O)N(R)₂, —C(O)OR, —C₁₋₆ aliphatic, or —O—C₁₋₆ aliphatic, wherein each of —C₁₋₆ aliphatic and —O—C₁₋₆ aliphatic is independently substituted 0-6 times by halogen, —CN, or —NO₂.

In some embodiments, Ring B is optionally substituted 1-4 times by —F, —Cl, —Br—, —S(O)₂NHCH₃, —S(O)NHCH₃, —C(O)N(CH₃)₂, —C(O)NHCH₃, —C(O)OH, —C(O)OCH₃, —CH₃, —OCH₃, or —C(CH₃)₃.

In some embodiments, Ring B is

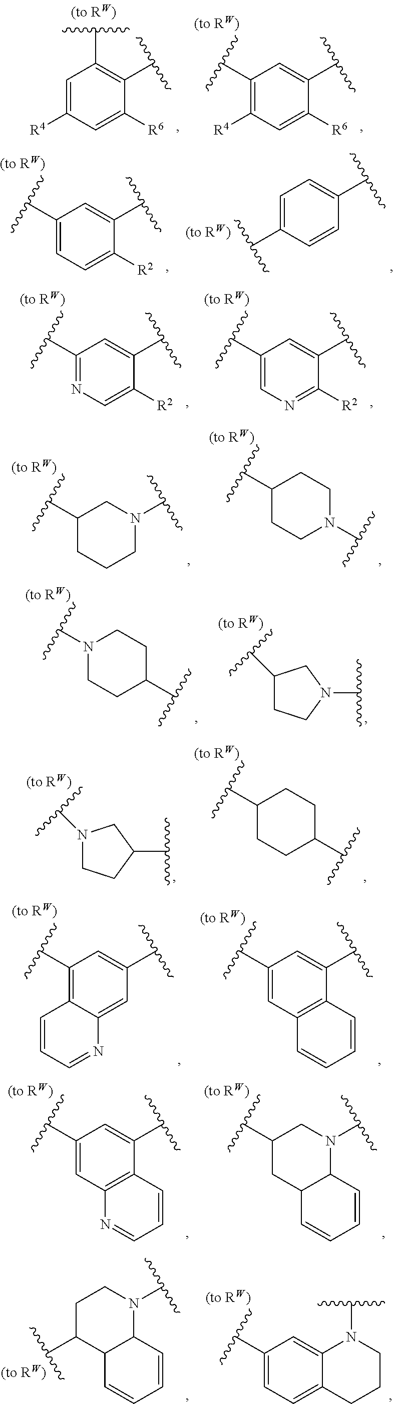

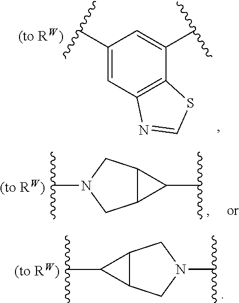

In some embodiments, Ring B is selected from those depicted in Table A, below.

As defined generally above, R² is —H, or an optionally substituted 4-, 5-, or 6-membered ring having 1, 2, 3, or 4 heteroatoms independently selected from nitrogen, oxygen, or sulfur.

In some embodiments, R² is —H.

In some embodiments, R² is an optionally substituted 4-, 5-, or 6-membered ring having 1, 2, 3, or 4 heteroatoms independently selected from nitrogen, oxygen, or sulfur. In some embodiments, R² is a 4-, 5-, or 6-membered ring having 1, 2, 3, or 4 heteroatoms independently selected from nitrogen, oxygen, or sulfur, optionally substituted 1-3 times by —C₁₋₆ alkyl.

In some embodiments, R² is

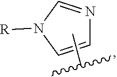

wherein R is as described herein. In some embodiments, R² is

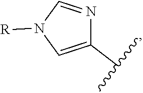

wherein R is as described herein.

In some embodiments, R² is

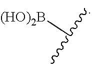

In some embodiments, R² is

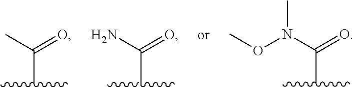

In some embodiments, R² is an optionally substituted 5-membered ring having 1, 2, 3, or 4 nitrogen. In some embodiments, R² is selected from

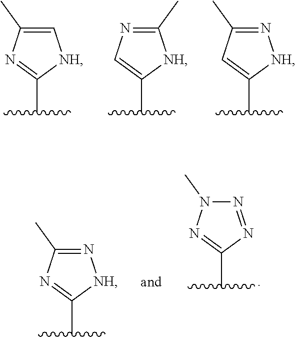

In some embodiments, R² is

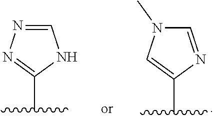

In some embodiments, R² is

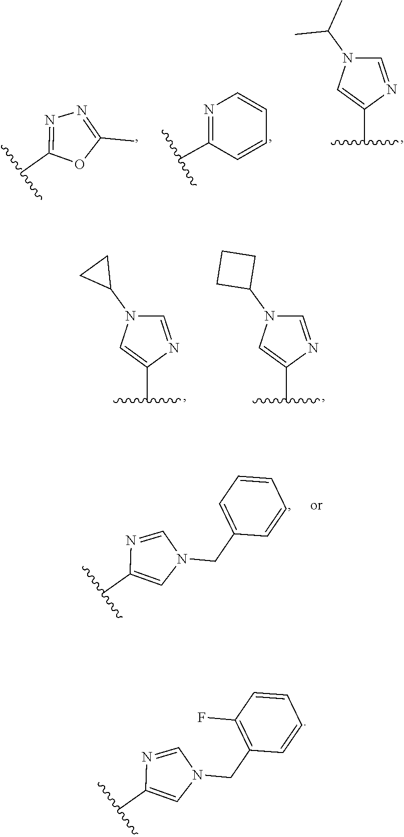

In some embodiments, R² is selected from those depicted in Table A, below.

As defined generally above, in some embodiments, R³ is —H.

In some embodiments, R³ is

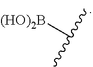 [SMILES: *B(O)O]

In some embodiments, R³ is

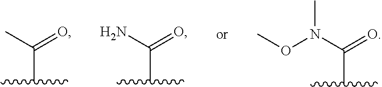

In some embodiments, R³ is selected from those depicted in Table A, below.

As defined generally above, R⁴ is —H, halogen, —S(O)₂N(R)₂, —S(O)N(R)₂, or —C(O)N(R)₂.

In some embodiments, R⁴ is —H, halogen, —S(O)₂N(R)₂, —S(O)N(R)₂, —C(O)N(R)₂, or —C(O)OR.

In some embodiments, R⁴ is —H.

In some embodiments, R⁴ is halogen. In some embodiments, R⁴ is —F. In some embodiments, R⁴ is —Cl. In some embodiments, R⁴ is —Br.

In some embodiments, R⁴ is —S(O)₂N(R)₂, —S(O)N(R)₂, or —C(O)N(R)₂. In some embodiments, R⁴ is —S(O)₂N(R)₂. In some embodiments, R⁴ is —S(O)N(R)₂. In some embodiments, R⁴ is —C(O)N(R)₂. In some embodiments, R⁴ is —S(O)₂NHCH₃.

In some embodiments, R⁴ is —S(O)NHCH₃, —C(O)N(CH₃)₂, —C(O)NHCH₃, —C(O)OH, or —C(O)OCH₃.

In some embodiments, R⁴ is

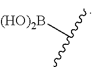 [SMILES: *B(O)O]

In some embodiments, R⁴ is

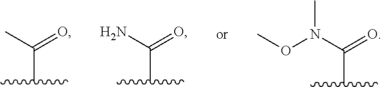

In some embodiments, R⁴ is selected from those depicted in Table A, below.

As defined generally above, R⁶ is —H or —C₁₋₆ aliphatic substituted 0, 1, 2, 3, 4, 5, or 6 times by -halogen, —CN, or —NO₂.

In some embodiments, R⁶ is —H, -halogen, —CN, —NO₂, —C₁₋₆ aliphatic, —OC₁₋₆ aliphatic, or a 4-, 5-, or 6-membered ring having 1, 2, 3, or 4 heteroatoms independently selected from nitrogen, oxygen, or sulfur optionally substituted 1-3 times by —C₁₋₆ aliphatic or —OC₁₋₆ aliphatic, wherein each of —C₁₋₆ aliphatic and —OC₁₋₆ aliphatic is independently substituted 0, 1, 2, 3, 4, 5, or 6 times by -halogen, —CN, or —NO₂.

In some embodiments, R⁶ is —H. In some embodiments, R⁶ is —F. In some embodiments, R⁶ is —Cl. In some embodiments, R⁶ is —Br. In some embodiments, R⁶ is —CN. In some embodiments, R⁶ is —NO₂.

In some embodiments, R⁶ is —C₁₋₆ aliphatic, substituted 0, 1, 2, 3, 4, 5, or 6 times by -halogen, —CN, or —NO₂. In some embodiments, R⁶ is unsubstituted —C₁₋₆ aliphatic. In some embodiments, R⁶ is —CH₃. In some embodiments, R⁶ is —C₁₋₆ aliphatic substituted 1, 2, 3, 4, 5, or 6 times by -halogen, —CN, or —NO₂. In some embodiments, R⁶ is —C₁₋₆ aliphatic substituted 1, 2, 3, 4, 5, or 6 times by —F. In some embodiments, R⁶ is —C₁₋₃ aliphatic substituted 1, 2, 3, 4, 5, or 6 times by —F. In some embodiments, R⁶ is —CF₃.

In some embodiments, R⁶ is —OC₁₋₆ aliphatic, substituted 0, 1, 2, 3, 4, 5, or 6 times by -halogen, —CN, or —NO₂. In some embodiments, R⁶ is unsubstituted —OC₁₋₆ aliphatic. In some embodiments, R⁶ is —OCH₃. In some embodiments, R⁶ is —OC₁₋₆ aliphatic substituted 1, 2, 3, 4, 5, or 6 times by -halogen, —CN, or —NO₂. In some embodiments, R⁶ is —OC₁₋₆ aliphatic substituted 1, 2, 3, 4, 5, or 6 times by —F. In some embodiments, R⁶ is —OC₁₋₃ aliphatic substituted 1, 2, 3, 4, 5, or 6 times by —F. In some embodiments, R⁶ is —OCF₃.

In some embodiments, R⁶ is a 4-, 5-, or 6-membered ring having 1, 2, 3, or 4 heteroatoms independently selected from nitrogen, oxygen, or sulfur optionally substituted 1-3 times by —C₁₋₆ aliphatic or —OC₁₋₆ aliphatic, wherein each of —C₁₋₆ aliphatic and —OC₁₋₆ aliphatic is independently substituted 0, 1, 2, 3, 4, 5, or 6 times by -halogen, —CN, or —NO₂. In some embodiments, R⁶ is a 5-membered ring having 1, 2, 3, or 4 nitrogen optionally substituted 1-3 times by —C₁₋₆ aliphatic. In some embodiments, R⁶ is

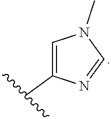 [SMILES: *c1cn(C)cn1]

In some embodiments, R⁶ is selected from those depicted in Table A, below.

As defined generally above, R^{w} is an optionally substituted 4-, 5-, or 6-membered ring having 1, 2, 3, or 4 heteroatoms independently selected from nitrogen, oxygen, or sulfur.

In some embodiments, R^{w} is an optionally substituted 4-, 5-, or 6-membered ring having 1, 2, 3, or 4 heteroatoms independently selected from nitrogen, oxygen, or sulfur. In some embodiments, R^{w} is a 4-, 5-, or 6-membered ring having 1, 2, 3, or 4 heteroatoms independently selected from nitrogen, oxygen, or sulfur, optionally substituted 1-3 times by —C₁₋₆ alkyl.

In some embodiments, R^{w} is

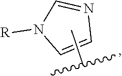

wherein R is as described herein. In some embodiments, R^{w} is

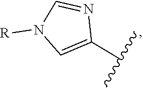

wherein R is as described herein.

In some embodiments, R^{w} is a 4-, 5-, or 6-membered ring having 1, 2, 3, or 4 heteroatoms independently selected from nitrogen, oxygen, or sulfur, optionally substituted 1-3 times by —C₁₋₆ alkyl. In some embodiments, R^{w} is an optionally substituted 5-membered ring having 1, 2, 3, or 4 nitrogen. In some embodiments, R^{w} is

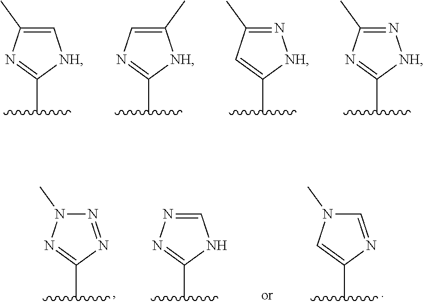

In some embodiments, R^{w} is

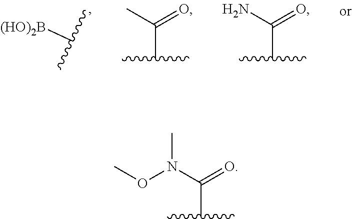

In some embodiments, R^{w} is

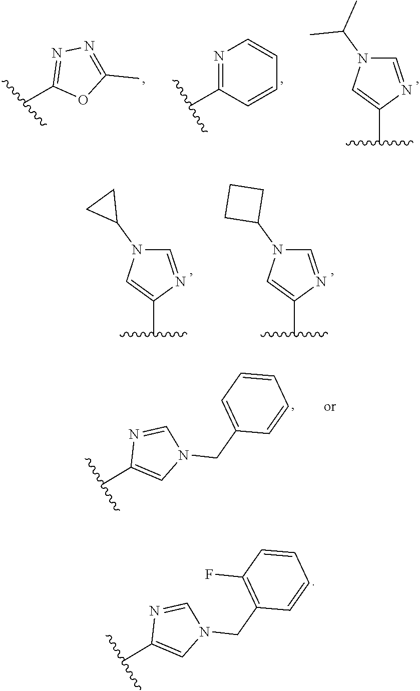

In some embodiments, R^{w} is selected from those depicted in Table A, below.

As defined generally above, R is independently —H or optionally substituted —C₁₋₆ aliphatic.

In some embodiments, R is —H.

In some embodiments, R is optionally substituted —C₁₋₆ aliphatic. In some embodiments, R is unsubstituted —C₁₋₆ aliphatic. In some embodiments, R is —CH₃. In some embodiments, R is —C₁₋₆ aliphatic substituted 1, 2, 3, 4, 5, or 6 times by -halogen, —CN, or —NO₂. In some embodiments, R is —C₁₋₆ aliphatic substituted 1, 2, 3, 4, 5, or 6 times by —F. In some embodiments, R is —C₁₋₃ aliphatic substituted 1, 2, 3, 4, 5, or 6 times by —F. In some embodiments, R is —CF₃.

In some embodiments, R is selected from those depicted in Table A, below.

In some embodiments, a TEAD inhibitor is a compound of Formula A-2:

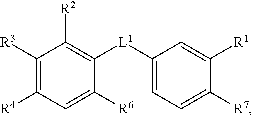

or a pharmaceutically acceptable salt thereof, wherein each of R¹, R², R³, R⁴, R⁶, R⁷, and L¹ is independently as defined and described in embodiments in Section of TEAD Inhibitors of Formulae A, and A-1 to A-50.

In some embodiments, the present invention provides a compound of formula A-2, or a pharmaceutically acceptable salt thereof, wherein:
(a):
   L¹ is —O— or —S—;
   R¹ is —C₁₋₆ aliphatic substituted 0, 1, 2, 3, 4, 5, or 6 times by halogen;
   R² is an optionally substituted 5-membered aromatic ring having 1, 2, 3, or 4 nitrogen;
   R³ is —H;
   R⁴ is —S(O)₂N(R)₂; —S(O)N(R)₂, or —C(O)N(R)₂, each R independently is selected —H and optionally substituted —C₁₋₆ aliphatic;
   R⁶ is —H or —C₁₋₆ aliphatic substituted 0, 1, 2, 3, 4, 5, or 6 times by halogen; and
   R⁷ is —H; or
(b):
   L¹ is —NH—;
   R¹ is —C₁₋₆ aliphatic substituted 1, 2, 3, 4, 5, or 6 times by halogen;
   R² is an optionally substituted 5-membered aromatic ring having 1, 2, 3, or 4 nitrogen;
   R³ is —H;
   R⁴ is —S(O)₂N(R)₂, —S(O)N(R)₂, or —C(O)N(R)₂, each R independently is selected from —H and optionally substituted —C₁₋₆ aliphatic;
   R⁶ is —C₁₋₆ aliphatic; and
   R⁷ is —H.

In some embodiments, a TEAD inhibitor is a compound of Formula:

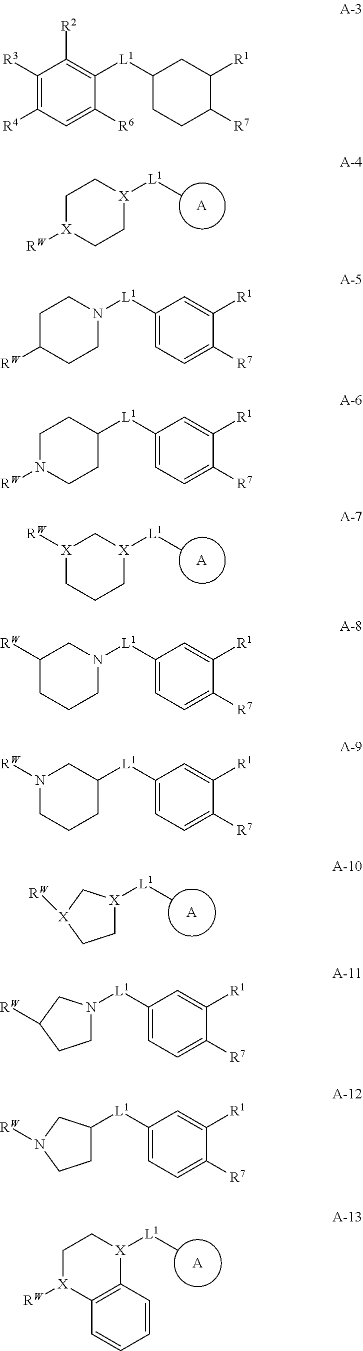

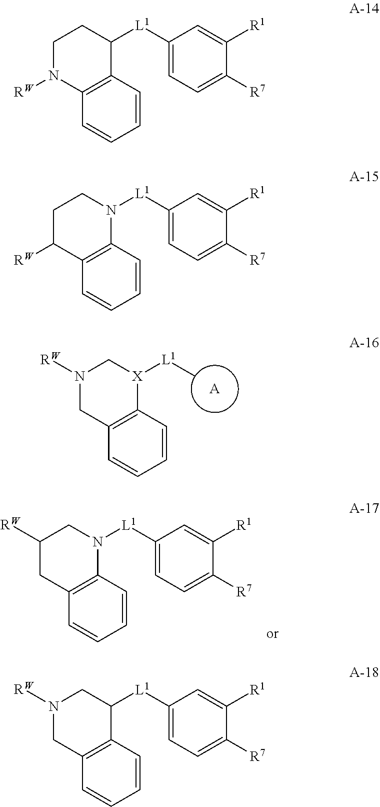

or a pharmaceutically acceptable salt thereof, wherein each of X is independently C or N, and each of Ring A, R^{w}, R¹, R², R³, R⁴, R⁶, R⁷, and L¹ is independently as defined and described in embodiments in the section of TEAD Inhibitors of Formulae A, and A-1 to A-50.

In some embodiments, a TEAD inhibitor is a compound of Formula A, or a pharmaceutically acceptable salt thereof, wherein Ring A is phenyl, a 6-membered monocyclic heteroaromatic ring having 1 or 2 nitrogen, or a 10-membered bicyclic heteroaromatic ring having 1-2 nitrogen; Ring B is phenyl or a 6-membered monocyclic heteroaromatic ring having 1 or 2 nitrogen; and each of R^{w} and L¹ is as defined above and described in embodiments herein, both singly and in combination.

In some embodiments, a TEAD inhibitor is a compound selected from the following:
i. Formula (A-19) or (A-20):

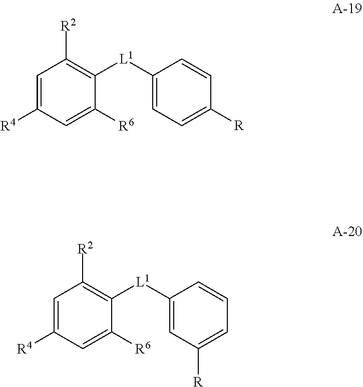

wherein L¹ is a C₂₋₆ bivalent straight or branched hydrocarbon chain wherein 1 methylene unit of the chain is replaced with —N(R)—, and each of R², R⁴, R⁶, and R is independently as defined and described in embodiments in the section of TEAD Inhibitors of Formulae A, and A-1 to A-50;
ii. Formula (A-21) or (A-22):

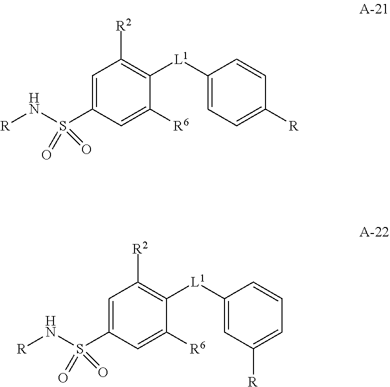

wherein L¹ is a C₂₋₆ bivalent straight or branched hydrocarbon chain wherein 1 methylene unit of the chain is replaced with —N(R)—, and each of R², R⁶, and R is independently as defined and described in embodiments in the section of TEAD Inhibitors of Formulae A, and A-1 to A-50;
iii. Formula (A-23) or (A-24):

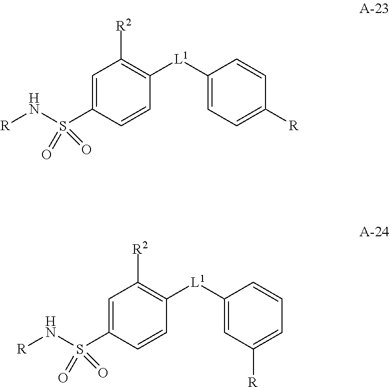

wherein L¹ is a C₂₋₆ bivalent straight or branched hydrocarbon chain wherein 1 methylene unit of the chain is replaced with —NH—, each of R² and R is independently as defined and described in embodiments in the section of TEAD Inhibitors of Formulae A, and A-1 to A-50;
iv. Formula (A-25) or (A-26):

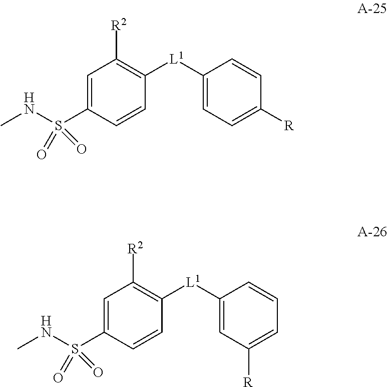

wherein L¹ is a C₂₋₆ bivalent straight or branched hydrocarbon chain wherein 1 methylene unit of the chain is replaced with —NH—, R is —C₁₋₃ aliphatic substituted 1, 2, 3, 4, 5, or 6 times by —F, and R² is as defined and described in embodiments in the section of TEAD Inhibitors of Formulae A, and A-1 to A-50;
v. Formula (A-27) or (A-28):

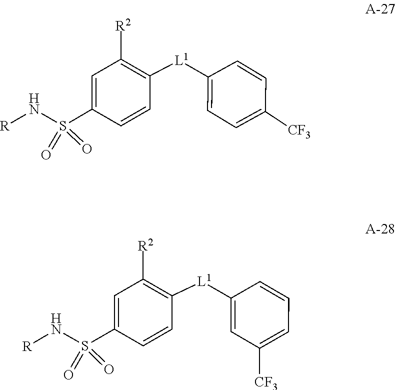

wherein L¹ is a C₂₋₆ bivalent straight hydrocarbon chain wherein 1 methylene unit of the chain is replaced with —NH—, R is optionally substituted —C₁₋₆ aliphatic, and R² is as defined and described in embodiments in the section of TEAD Inhibitors of Formulae A, and A-1 to A-50;
vi. Formula (A-29) or (A-30):

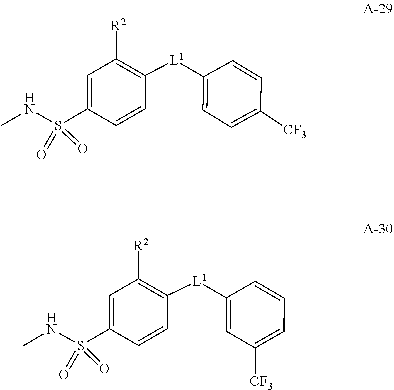

wherein L¹ is a C₂₋₆ bivalent straight hydrocarbon chain wherein 1 methylene unit of the chain is replaced with —NH—, and R² is as defined and described in embodiments in the section of TEAD Inhibitors of Formulae A, and A-1 to A-50;
vii. Formula (A-31) or (A-32):

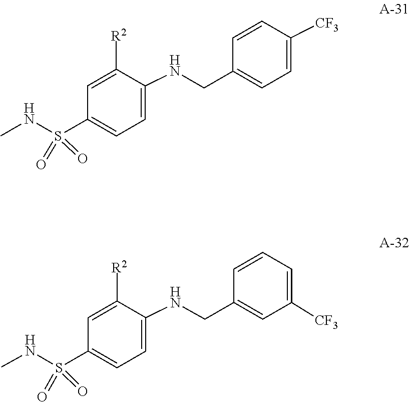

wherein R² is an optionally substituted 5-membered ring having 1, 2, 3, or 4 nitrogen; viii. Formula (A-33) or (A-34):

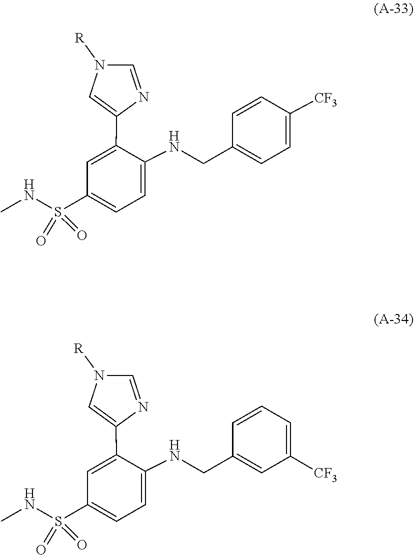

wherein R is independently as defined and described in embodiments in the section of TEAD Inhibitors of Formulae A, and A-1 to A-50;
ix. Formula (A-35) or (A-36):

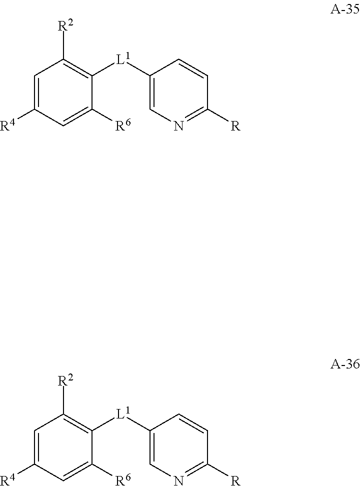

wherein L¹ is a C₁₋₆ bivalent straight or branched hydrocarbon chain wherein 1 methylene unit of the chain is replaced with —N(R)—, and each of R², R⁴, R⁶, and R is independently as defined and described in embodiments in the section of TEAD Inhibitors of Formulae A, and A-1 to A-50;
x. Formula (A-37) or (A-38):

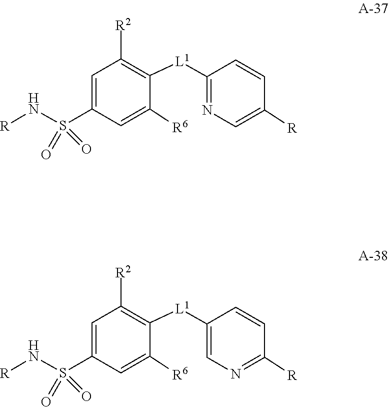

wherein L¹ is a C₁₋₆ bivalent straight or branched hydrocarbon chain wherein 1 methylene unit of the chain is replaced with —N(R)—, and each of R², R⁶, and R is independently as defined and described in embodiments in the section of TEAD Inhibitors of Formulae A, and A-1 to A-50;
xi. Formula (A-39) or (A-40):

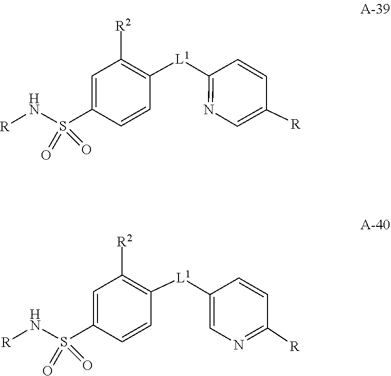

wherein L¹ is a C₁₋₆ bivalent straight or branched hydrocarbon chain wherein 1 methylene unit of the chain is replaced with —NH—, each of R² and R is independently as defined and described in embodiments in the section of TEAD Inhibitors of Formulae A, and A-1 to A-50;
xii. Formula (A-41) or (A-42):

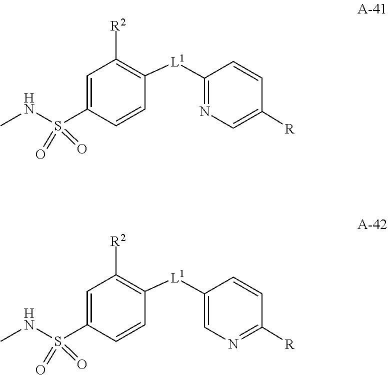

wherein L¹ is a C₁₋₆ bivalent straight or branched hydrocarbon chain wherein 1 methylene unit of the chain is replaced with —NH—, R is —C₁₋₃ aliphatic substituted 1, 2, 3, 4, 5, or 6 times by —F, and R² is as defined and described in embodiments in the section of TEAD Inhibitors of Formulae A, and A-1 to A-50;
xiii. Formula (A-43) or (A-44):

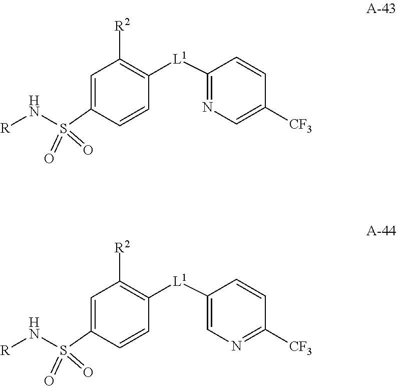

wherein L¹ is a C₁₋₆ bivalent straight hydrocarbon chain wherein 1 methylene unit of the chain is replaced with —NH—, R is optionally substituted —C₁₋₆ aliphatic, and R² is as defined and described in embodiments in the section of TEAD Inhibitors of Formulae A, and A-1 to A-50;
xiv. Formula (A-45) or (A-46):

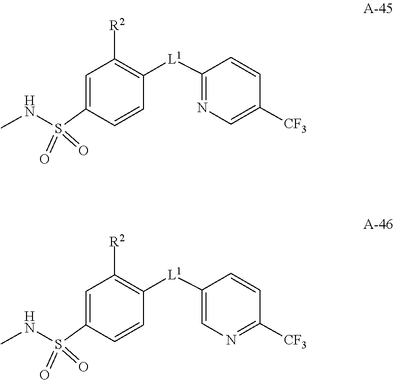

wherein L¹ is a C₁₋₆ bivalent straight hydrocarbon chain wherein 1 methylene unit of the chain is replaced with —NH—, and R² is as defined and described in embodiments in the section of TEAD Inhibitors of Formulae A, and A-1 to A-50;
xv. Formula (A-47) or (A-48):

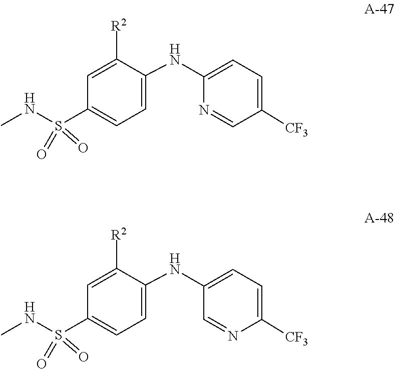

wherein R² is an optionally substituted 5-membered ring having 1, 2, 3, or 4 nitrogen; or
xvi. Formula (A-49) or (A-50):

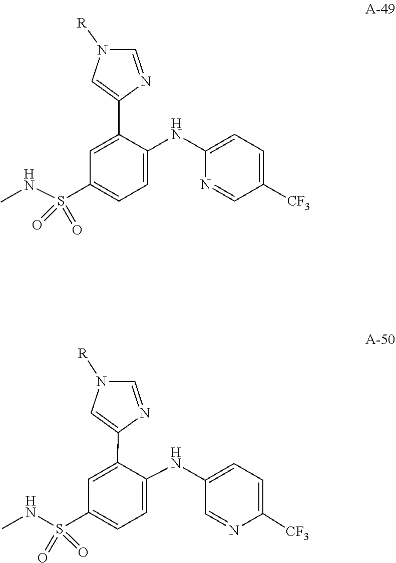

wherein R is independently as defined and described in embodiments in the section of TEAD Inhibitors of Formulae A, and A-1 to A-50.

In some embodiments, a TEAD inhibitor is selected from those listed in Table A, or a pharmaceutically acceptable salt thereof.

TABLE A
Exemplified TEAD Inhibitors

| | |
|---|---|
| 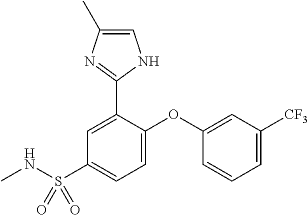 [SMILES: CNS(=O)(=O)c1ccc(Oc2cccc(C(F)(F)F)c2)c(-c2nc(C)c[nH]2)c1] | T-A-1 |
| 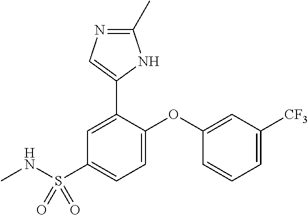 [SMILES: CNS(=O)(=O)c1ccc(Oc2cccc(C(F)(F)F)c2)c(-c2cnc(C)[nH]2)c1] | T-A-2 |
| 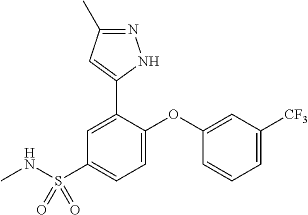 [SMILES: CNS(=O)(=O)c1ccc(Oc2cccc(C(F)(F)F)c2)c(-c2cc(C)n[nH]2)c1] | T-A-3 |
| 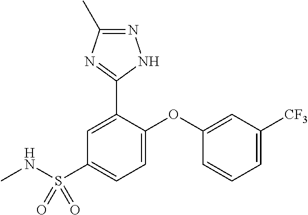 [SMILES: CNS(=O)(=O)c1ccc(Oc2cccc(C(F)(F)F)c2)c(-c2nc(C)n[nH]2)c1] | T-A-4 |
| 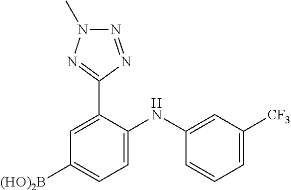 [SMILES: Cn1nnc(-c2cc(B(O)O)ccc2Nc2cccc(C(F)(F)F)c2)n1] | T-A-5 |
| 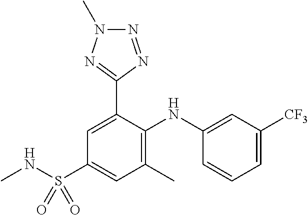 [SMILES: CNS(=O)(=O)c1cc(C)c(Nc2cccc(C(F)(F)F)c2)c(-c2nnn(C)n2)c1] | T-A-6 |
| 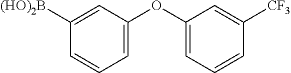 [SMILES: OB(O)c1cccc(Oc2cccc(C(F)(F)F)c2)c1] | T-A-7 |
| 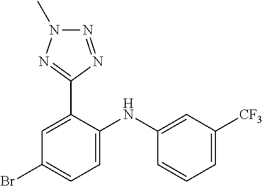 [SMILES: Cn1nnc(-c2cc(Br)ccc2Nc2cccc(C(F)(F)F)c2)n1] | T-A-8 |
| 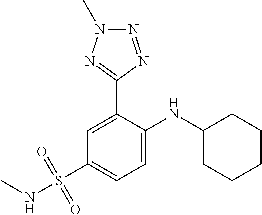 [SMILES: CNS(=O)(=O)c1ccc(NC2CCCCC2)c(-c2nnn(C)n2)c1] | T-A-9 |
| 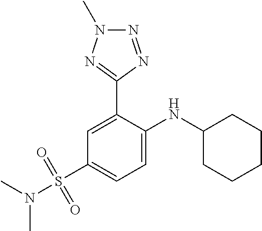 [SMILES: CN(C)S(=O)(=O)c1ccc(NC2CCCCC2)c(-c2nnn(C)n2)c1] | T-A-10 |
| 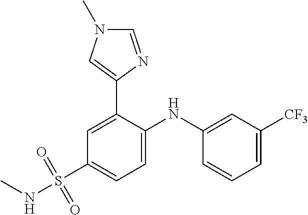 [SMILES: CNS(=O)(=O)c1ccc(Nc2cccc(C(F)(F)F)c2)c(-c2cn(C)cn2)c1] | T-A-11 |
| 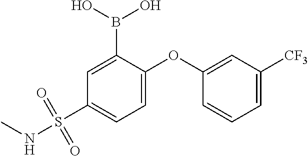 [SMILES: CNS(=O)(=O)c1ccc(Oc2cccc(C(F)(F)F)c2)c(B(O)O)c1] | T-A-12 |
| 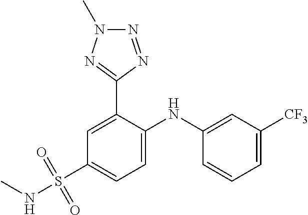 [SMILES: CNS(=O)(=O)c1ccc(Nc2cccc(C(F)(F)F)c2)c(-c2nnn(C)n2)c1] | T-A-13 |
| 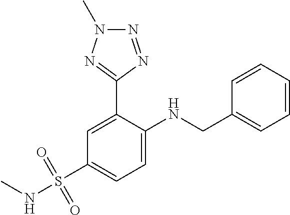 [SMILES: CNS(=O)(=O)c1ccc(NCc2ccccc2)c(-c2nnn(C)n2)c1] | T-A-14 |
| 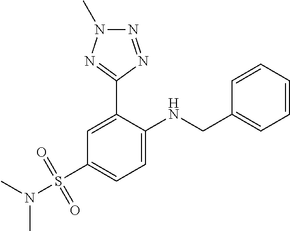 [SMILES: CN(C)S(=O)(=O)c1ccc(NCc2ccccc2)c(-c2nnn(C)n2)c1] | T-A-15 |
| 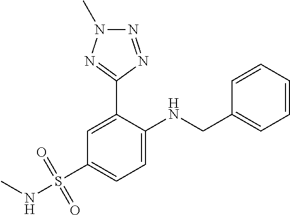 [SMILES: CNS(=O)(=O)c1ccc(NCc2ccccc2)c(-c2nnn(C)n2)c1] | T-A-16 |
| 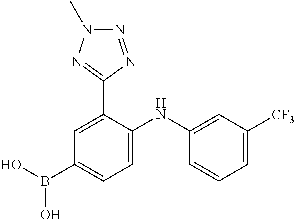 [SMILES: Cn1nnc(-c2cc(B(O)O)ccc2Nc2cccc(C(F)(F)F)c2)n1] | T-A-17 |
| 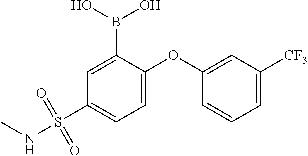 [SMILES: CNS(=O)(=O)c1ccc(Oc2cccc(C(F)(F)F)c2)c(B(O)O)c1] | T-A-18 |
| 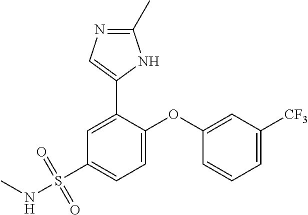 [SMILES: CNS(=O)(=O)c1ccc(Oc2cccc(C(F)(F)F)c2)c(-c2cnc(C)[nH]2)c1] | T-A-19 |
| 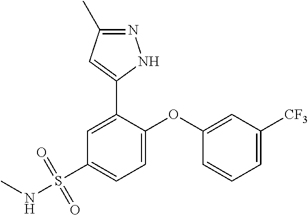 [SMILES: CNS(=O)(=O)c1ccc(Oc2cccc(C(F)(F)F)c2)c(-c2cc(C)n[nH]2)c1] | T-A-20 |
| 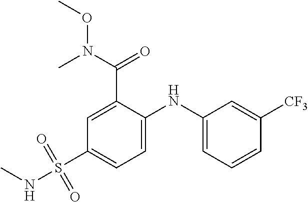 [SMILES: CNS(=O)(=O)c1ccc(Nc2cccc(C(F)(F)F)c2)c(C(=O)N(C)OC)c1] | T-A-21 |
| 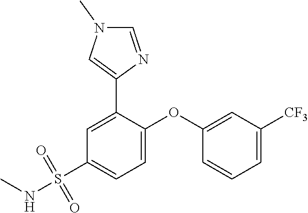 [SMILES: CNS(=O)(=O)c1ccc(Oc2cccc(C(F)(F)F)c2)c(-c2cn(C)cn2)c1] | T-A-22 |
| 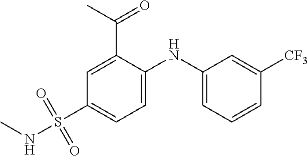 [SMILES: CNS(=O)(=O)c1ccc(Nc2cccc(C(F)(F)F)c2)c(C(C)=O)c1] | T-A-23 |
| 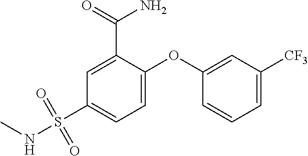 [SMILES: CNS(=O)(=O)c1ccc(Oc2cccc(C(F)(F)F)c2)c(C(N)=O)c1] | T-A-24 |
| 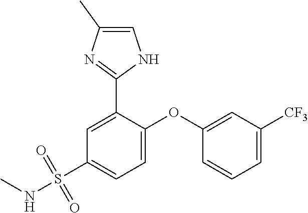 [SMILES: CNS(=O)(=O)c1ccc(Oc2cccc(C(F)(F)F)c2)c(-c2nc(C)c[nH]2)c1] | T-A-25 |
| 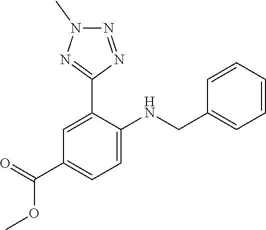 [SMILES: COC(=O)c1ccc(NCc2ccccc2)c(-c2nnn(C)n2)c1] | T-A-26 |
| 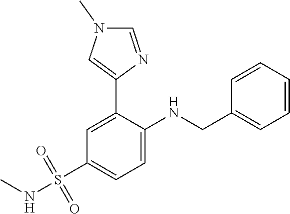 [SMILES: CNS(=O)(=O)c1ccc(NCc2ccccc2)c(-c2cn(C)cn2)c1] | T-A-27 |
| 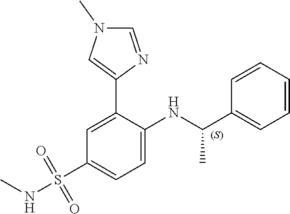 | T-A-28 |
| 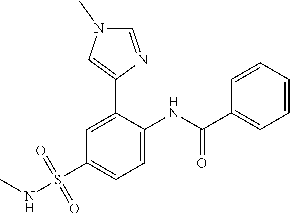 [SMILES: CNS(=O)(=O)c1ccc(NC(=O)c2ccccc2)c(-c2cn(C)cn2)c1] | T-A-29 |
| 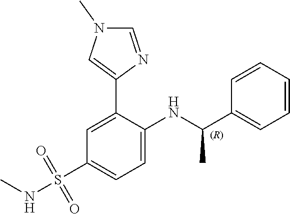 | T-A-30 |
| 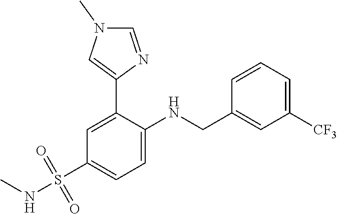 [SMILES: CNS(=O)(=O)c1ccc(NCc2cccc(C(F)(F)F)c2)c(-c2cn(C)cn2)c1] | T-A-31 |
| 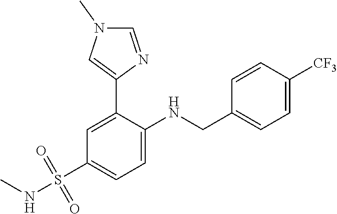 [SMILES: CNS(=O)(=O)c1ccc(NCc2ccc(C(F)(F)F)cc2)c(-c2cn(C)cn2)c1] | T-A-32 |
| 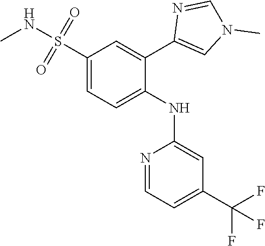 [SMILES: CNS(=O)(=O)c1ccc(Nc2cc(C(F)(F)F)ccn2)c(-c2cn(C)cn2)c1] | T-A-33 |
| 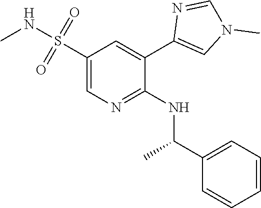 [SMILES: CNS(=O)(=O)c1cnc(N[C@@H](C)c2ccccc2)c(-c2cn(C)cn2)c1] | T-A-34 |
| 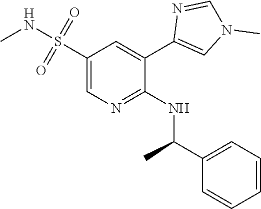 [SMILES: CNS(=O)(=O)c1cnc(N[C@H](C)c2ccccc2)c(-c2cn(C)cn2)c1] | T-A-35 |
| 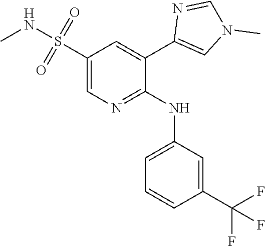 [SMILES: CNS(=O)(=O)c1cnc(Nc2cccc(C(F)(F)F)c2)c(-c2cn(C)cn2)c1] | T-A-36 |
| 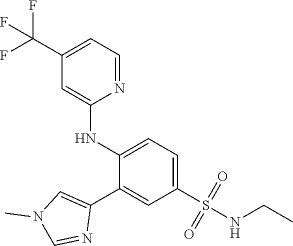 [SMILES: CCNS(=O)(=O)c1ccc(Nc2cc(C(F)(F)F)ccn2)c(-c2cn(C)cn2)c1] | T-A-37 |
| 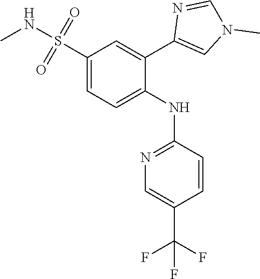 | T-A-38 |
| 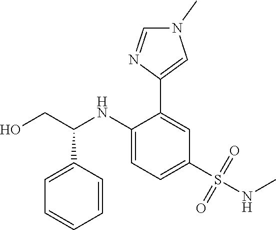 [SMILES: CNS(=O)(=O)c1ccc(N[C@@H](CO)c2ccccc2)c(-c2cn(C)cn2)c1] | T-A-39 |
| 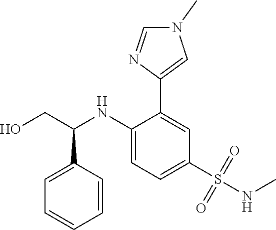 [SMILES: CNS(=O)(=O)c1ccc(N[C@H](CO)c2ccccc2)c(-c2cn(C)cn2)c1] | T-A-40 |
| 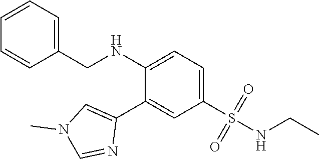 [SMILES: CCNS(=O)(=O)c1ccc(NCc2ccccc2)c(-c2cn(C)cn2)c1] | T-A-41 |
| 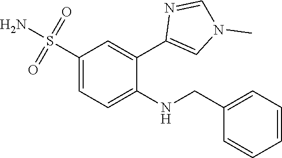 [SMILES: Cn1cnc(-c2cc(S(N)(=O)=O)ccc2NCc2ccccc2)c1] | T-A-42 |
| 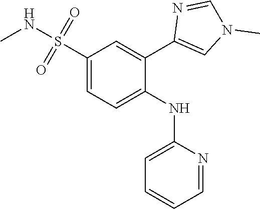 | T-A-43 |
| 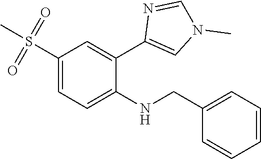 [SMILES: Cn1cnc(-c2cc(S(C)(=O)=O)ccc2NCc2ccccc2)c1] | T-A-44 |
| 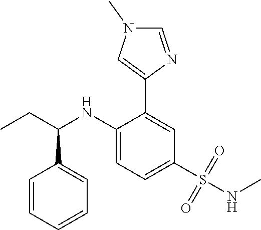 [SMILES: CC[C@@H](Nc1ccc(S(=O)(=O)NC)cc1-c1cn(C)cn1)c1ccccc1] | T-A-45 |
| 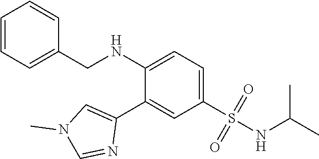 [SMILES: CC(C)NS(=O)(=O)c1ccc(NCc2ccccc2)c(-c2cn(C)cn2)c1] | T-A-46 |
| 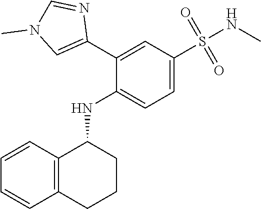 [SMILES: CNS(=O)(=O)c1ccc(N[C@@H]2CCCc3ccccc32)c(-c2cn(C)cn2)c1] | T-A-47 |
| 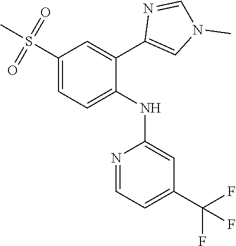 [SMILES: Cn1cnc(-c2cc(S(C)(=O)=O)ccc2Nc2cc(C(F)(F)F)ccn2)c1] | T-A-48 |
| 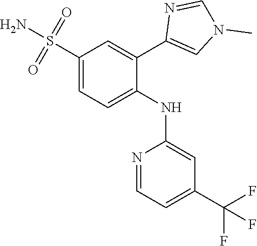 [SMILES: Cn1cnc(-c2cc(S(N)(=O)=O)ccc2Nc2cc(C(F)(F)F)ccn2)c1] | T-A-49 |
| 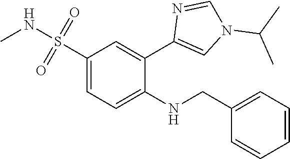 [SMILES: CNS(=O)(=O)c1ccc(NCc2ccccc2)c(-c2cn(C(C)C)cn2)c1] | T-A-50 |
| 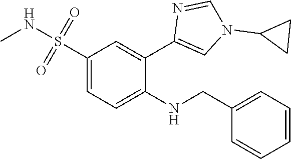 [SMILES: CNS(=O)(=O)c1ccc(NCc2ccccc2)c(-c2cn(C3CC3)cn2)c1] | T-A-51 |
| 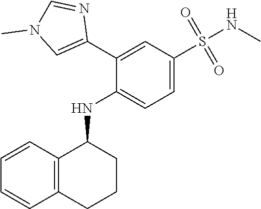 [SMILES: CNS(=O)(=O)c1ccc(N[C@H]2CCCc3ccccc32)c(-c2cn(C)cn2)c1] | T-A-52 |
| 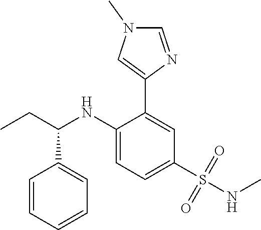 [SMILES: CC[C@H](Nc1ccc(S(=O)(=O)NC)cc1-c1cn(C)cn1)c1ccccc1] | T-A-53 |
| 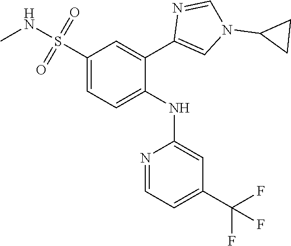 [SMILES: CNS(=O)(=O)c1ccc(Nc2cc(C(F)(F)F)ccn2)c(-c2cn(C3CC3)cn2)c1] | T-A-54 |
| 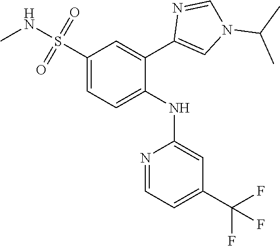 [SMILES: CNS(=O)(=O)c1ccc(Nc2cc(C(F)(F)F)ccn2)c(-c2cn(C(C)C)cn2)c1] | T-A-55 |
| 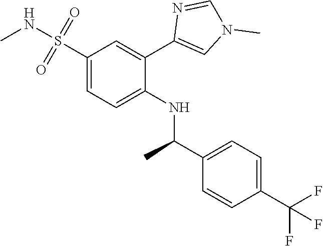 [SMILES: CNS(=O)(=O)c1ccc(N[C@H](C)c2ccc(C(F)(F)F)cc2)c(-c2cn(C)cn2)c1] | T-A-56 |
| 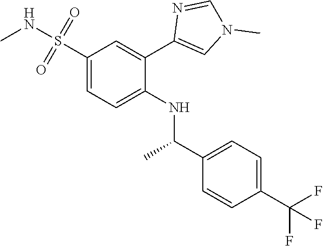 [SMILES: CNS(=O)(=O)c1ccc(N[C@@H](C)c2ccc(C(F)(F)F)cc2)c(-c2cn(C)cn2)c1] | T-A-57 |
| 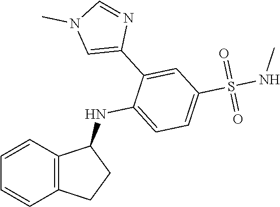 [SMILES: CNS(=O)(=O)c1ccc(N[C@H]2CCc3ccccc32)c(-c2cn(C)cn2)c1] | T-A-58 |
| 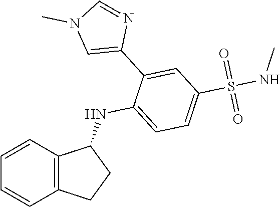 [SMILES: CNS(=O)(=O)c1ccc(N[C@@H]2CCc3ccccc32)c(-c2cn(C)cn2)c1] | T-A-59 |
| 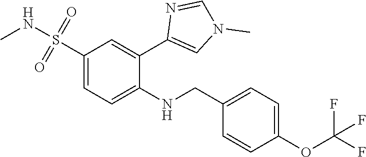 [SMILES: CNS(=O)(=O)c1ccc(NCc2ccc(OC(F)(F)F)cc2)c(-c2cn(C)cn2)c1] | T-A-60 |
| 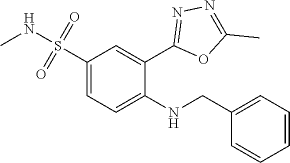 [SMILES: CNS(=O)(=O)c1ccc(NCc2ccccc2)c(-c2nnc(C)o2)c1] | T-A-61 |
| 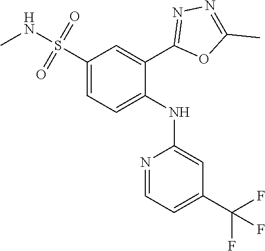 [SMILES: CNS(=O)(=O)c1ccc(Nc2cc(C(F)(F)F)ccn2)c(-c2nnc(C)o2)c1] | T-A-62 |
| 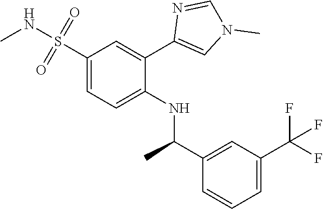 [SMILES: CNS(=O)(=O)c1ccc(N[C@H](C)c2cccc(C(F)(F)F)c2)c(-c2cn(C)cn2)c1] | T-A-63 |
| 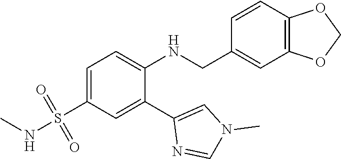 [SMILES: CNS(=O)(=O)c1ccc(NCc2ccc3c(c2)OCO3)c(-c2cn(C)cn2)c1] | T-A-64 |
| 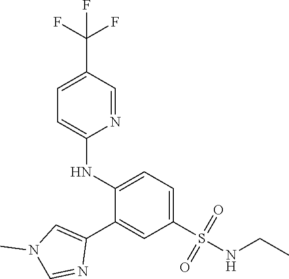 [SMILES: CCNS(=O)(=O)c1ccc(Nc2ccc(C(F)(F)F)cn2)c(-c2cn(C)cn2)c1] | T-A-65 |
| 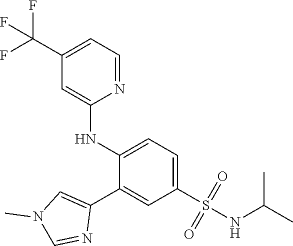 [SMILES: CC(C)NS(=O)(=O)c1ccc(Nc2cc(C(F)(F)F)ccn2)c(-c2cn(C)cn2)c1] | T-A-66 |
| 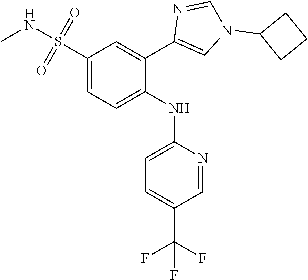 [SMILES: CNS(=O)(=O)c1ccc(Nc2ccc(C(F)(F)F)cn2)c(-c2cn(C3CCC3)cn2)c1] | T-A-67 |
| 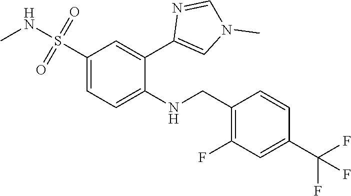 [SMILES: CNS(=O)(=O)c1ccc(NCc2ccc(C(F)(F)F)cc2F)c(-c2cn(C)cn2)c1] | T-A-68 |
| 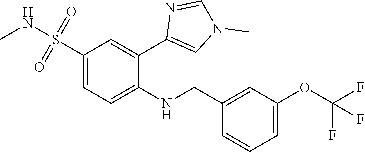 [SMILES: CNS(=O)(=O)c1ccc(NCc2cccc(OC(F)(F)F)c2)c(-c2cn(C)cn2)c1] | T-A-69 |
| 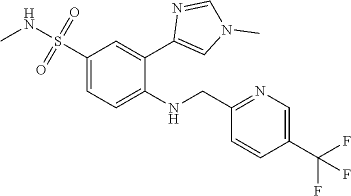 [SMILES: CNS(=O)(=O)c1ccc(NCc2ccc(C(F)(F)F)cn2)c(-c2cn(C)cn2)c1] | T-A-70 |
| 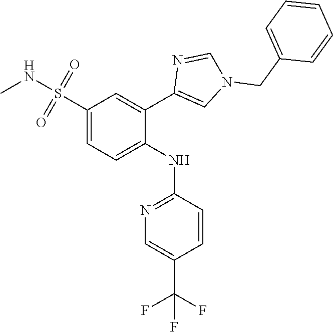 [SMILES: CNS(=O)(=O)c1ccc(Nc2ccc(C(F)(F)F)cn2)c(-c2cn(Cc3ccccc3)cn2)c1] | T-A-71 |
| 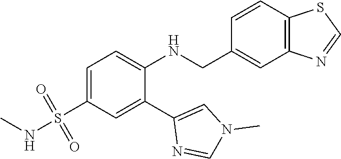 [SMILES: CNS(=O)(=O)c1ccc(NCc2ccc3scnc3c2)c(-c2cn(C)cn2)c1] | T-A-72 |
| 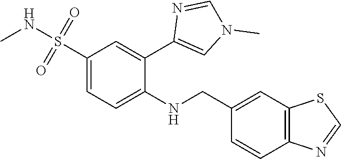 [SMILES: CNS(=O)(=O)c1ccc(NCc2ccc3ncsc3c2)c(-c2cn(C)cn2)c1] | T-A-73 |
| 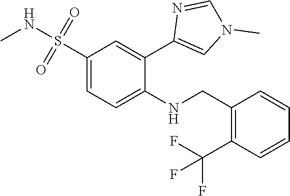 [SMILES: CNS(=O)(=O)c1ccc(NCc2ccccc2C(F)(F)F)c(-c2cn(C)cn2)c1] | T-A-74 |
| 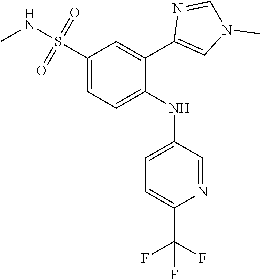 | T-A-75 |
| 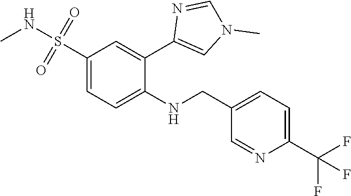 [SMILES: CNS(=O)(=O)c1ccc(NCc2ccc(C(F)(F)F)nc2)c(-c2cn(C)cn2)c1] | T-A-76 |
| 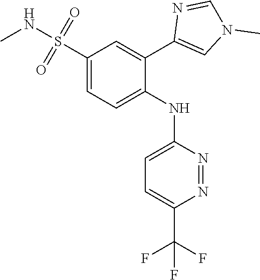 | T-A-77 |
| 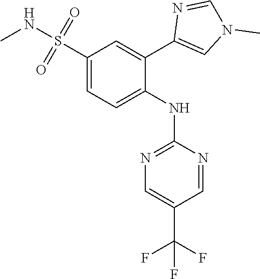 | T-A-78 |
| 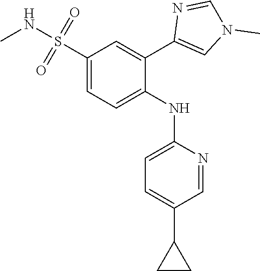 | T-A-79 |
| 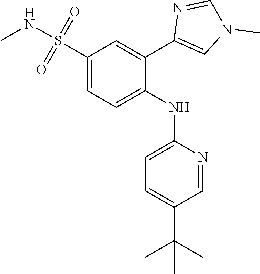 | T-A-80 |
| 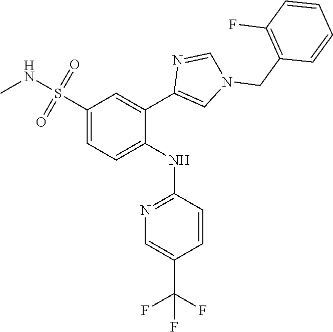 [SMILES: CNS(=O)(=O)c1ccc(Nc2ccc(C(F)(F)F)cn2)c(-c2cn(Cc3ccccc3F)cn2)c1] | T-A-81 |
| 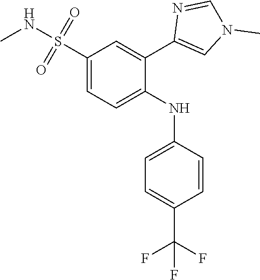 | T-A-82 |
| 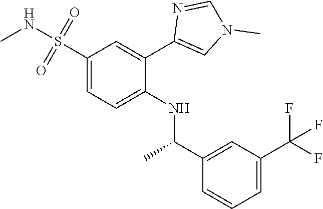 [SMILES: CNS(=O)(=O)c1ccc(N[C@@H](C)c2cccc(C(F)(F)F)c2)c(-c2cn(C)cn2)c1] | T-A-83 |
| 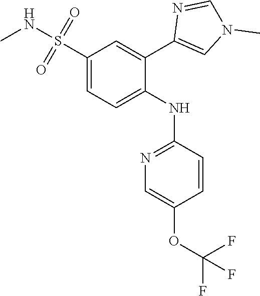 | T-A-84 |
| 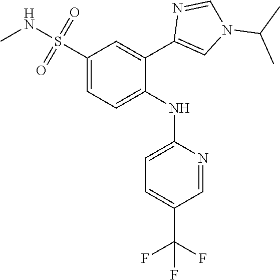 [SMILES: CNS(=O)(=O)c1ccc(Nc2ccc(C(F)(F)F)cn2)c(-c2cn(C(C)C)cn2)c1] | T-A-85 |
| 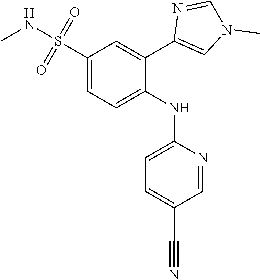 | T-A-86 |
| 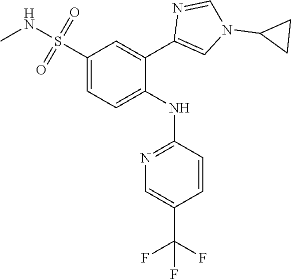 [SMILES: CNS(=O)(=O)c1ccc(Nc2ccc(C(F)(F)F)cn2)c(-c2cn(C3CC3)cn2)c1] | T-A-87 |
| 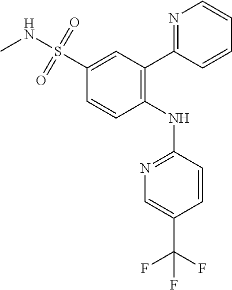 [SMILES: CNS(=O)(=O)c1ccc(Nc2ccc(C(F)(F)F)cn2)c(-c2ccccn2)c1] | T-A-88 |
| 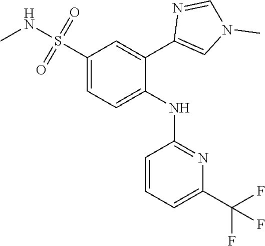 [SMILES: CNS(=O)(=O)c1ccc(Nc2cccc(C(F)(F)F)n2)c(-c2cn(C)cn2)c1] | T-A-89 |
| 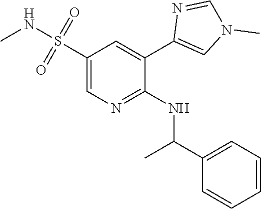 [SMILES: CNS(=O)(=O)c1cnc(NC(C)c2ccccc2)c(-c2cn(C)cn2)c1] | T-A-90 |
| 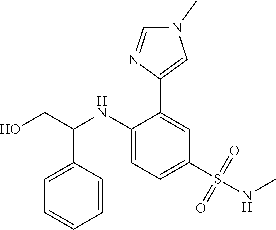 [SMILES: CNS(=O)(=O)c1ccc(NC(CO)c2ccccc2)c(-c2cn(C)cn2)c1] | T-A-91 |
| 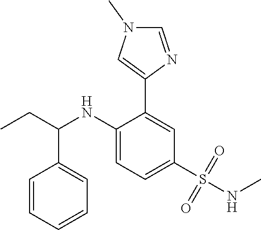 [SMILES: CCC(Nc1ccc(S(=O)(=O)NC)cc1-c1cn(C)cn1)c1ccccc1] | T-A-92 |
| 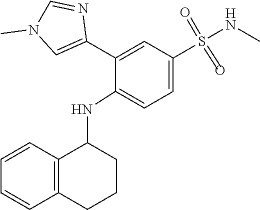 | T-A-93 |
| 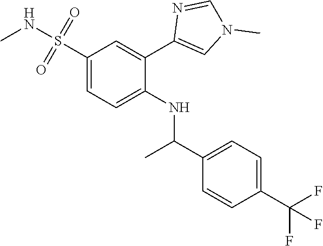 [SMILES: CNS(=O)(=O)c1ccc(NC(C)c2ccc(C(F)(F)F)cc2)c(-c2cn(C)cn2)c1] | T-A-94 |
| 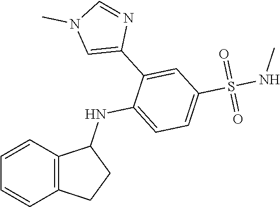 [SMILES: CNS(=O)(=O)c1ccc(NC2CCc3ccccc32)c(-c2cn(C)cn2)c1] | T-A-95 |
| 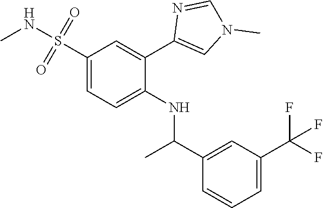 [SMILES: CNS(=O)(=O)c1ccc(NC(C)c2cccc(C(F)(F)F)c2)c(-c2cn(C)cn2)c1] | T-A-96 |

### 2. TEAD Inhibitors of Formulae B, and B-1 to B-34

In certain embodiments, a TEAD inhibitor is selected from those as described in WO 2020/243423, the contents of which are herein incorporated by reference in their entirety.

In certain embodiments, a TEAD inhibitor is a compound of Formula B:

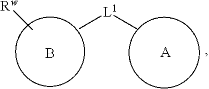

or a pharmaceutically acceptable salt thereof, wherein
L¹ is a covalent bond, or a C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—;
Ring A is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur;
Ring B is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, a 8-10 membered bicyclic heteroaromatic ring having 1-5 heteroatoms independently selected from nitrogen, oxygen, or sulfur;
R^{w} is a warhead group; wherein when R^{w} is a saturated or partially unsaturated monocyclic carbocyclic or heterocyclic ring, it optionally forms a spiro bicyclic ring with Ring B; and
each R is independently —H or optionally substituted —C₁₋₆ aliphatic.

In certain embodiments, a TEAD inhibitor is a compound of formula B-1

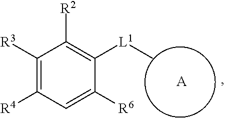

or a pharmaceutically acceptable salt thereof, wherein
L¹ is C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—;
Ring A is a 4-, 5-, or 6-membered saturated or partially unsaturated monocyclic carbocyclic ring, phenyl, a 4-, 5-, or 6-membered saturated or partially unsaturated monocyclic heterocyclic ring having 1-2 heteroatoms independently selected from nitrogen, oxygen, or sulfur, or a 5-6 membered monocyclic heteroaromatic ring having 1, 2, 3, or 4 heteroatoms independently selected from nitrogen, oxygen, or sulfur, wherein Ring A is optionally substituted 1-2 times by halogen, —CN, —NO₂, or —C₁₋₆ aliphatic substituted 0-6 times by halogen, —CN, or —NO₂;
R² is —H, or a warhead group;
R³ is —H or a warhead group;
R⁴ is —H, halogen, —S(O)₂N(R)₂, —S(O)N(R)₂, —C(O)N(R)₂, or a warhead group;
R⁶ is —H or —C₁₋₆ aliphatic substituted 0-6 times by halogen, —CN, or —NO₂; and each R is independently —H or optionally substituted —C₁₋₆ aliphatic.

As defined generally above, L¹ is C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is a covalent bond, or a C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is a covalent bond.

In some embodiments, L¹ is C₁₋₆ bivalent straight or branched hydrocarbon chain wherein 1, 2, or 3 methylene units of the chain are independently and optionally replaced with —O—, —CH(OR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, or —N(R)C(O)N(R)—.

In some embodiments, L¹ is C₁₋₆ bivalent straight or branched hydrocarbon chain wherein 1, 2, or 3 methylene units of the chain are optionally replaced with —CH(SR)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is C₁₋₆ bivalent straight or branched hydrocarbon chain wherein 1, 2, or 3 methylene units of the chain are independently and optionally replaced with —O—, —S—, or —N(R)—.

In some embodiments, L¹ is C₁₋₆ bivalent straight or branched hydrocarbon chain wherein 1, 2, or 3 methylene units of the chain are independently and optionally replaced with —CH(OR)—, —CH(SR)—, or —CH(N(R)₂)—.

In some embodiments, L¹ is C₁₋₆ bivalent straight or branched hydrocarbon chain wherein 1, 2, or 3 methylene units of the chain are independently and optionally replaced with —C(O)—, —C(O)O—, —OC(O)—, —SO—, —SO₂—, —C(S)—, —C(S)O—, or —OC(S)—.

In some embodiments, L¹ is C₁₋₆ bivalent straight or branched hydrocarbon chain wherein 1, 2, or 3 methylene units of the chain are independently and optionally replaced with —C(O)N(R)—, —(R)NC(O)—, —OC(O)N(R)—, —(R)NC(O)O—, —N(R)C(O)N(R)—, —SO₂N(R)—, —(R)NSO₂—, —C(S)N(R)—, —(R)NC(S)—, or —(R)NC(S)N(R)—.

In some embodiments, L¹ is —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is —O—, —CH(OR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, or —N(R)C(O)N(R)—.

In some embodiments, L¹ is —CH(SR)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—.

In some embodiments, L¹ is —O—, —S—, or —N(R)—. In some embodiments, L¹ is —O—. In some embodiments, L¹ is —S—. In some embodiments, L¹ is —N(R)—. In some embodiments, L¹ is —NH—.

In some embodiments, L¹ is —CH(OR)—, —CH(SR)—, or —CH(N(R)₂)—. In some embodiments, L¹ is —CH(OR)—. In some embodiments, L¹ is —CH(SR)—. In some embodiments, L¹ is —CH(N(R)₂)—.

In some embodiments, L¹ is —C(O)—, —C(O)O—, —OC(O)—, —SO—, —SO₂—, —C(S)—, —C(S)O—, or —OC(S)—. In some embodiments, L¹ is —C(O)—. In some embodiments, L¹ is —C(O)O—. In some embodiments, L¹ is —OC(O)—. In some embodiments, L¹ is —SO—. In some embodiments, L¹ is —SO₂—. In some embodiments, L¹ is —C(S)—. In some embodiments, L¹ is —C(S)O—. In some embodiments, L¹ is —OC(S)—.

In some embodiments, L¹ is —C(O)N(R)—, —(R)NC(O)—, —OC(O)N(R)—, —(R)NC(O)O—, —N(R)C(O)N(R)—, —SO₂N(R)—, —(R)NSO₂—, —C(S)N(R)—, —(R)NC(S)—, or —(R)NC(S)N(R)—. In some embodiments, L¹ is —C(O)N(R)—. In some embodiments, L¹ is —(R)NC(O)—. In some embodiments, L¹ is —OC(O)N(R)—. In some embodiments, L¹ is —(R)NC(O)O—. In some embodiments, L¹ is —N(R)C(O)N(R)—. In some embodiments, L¹ is —SO₂N(R)—. In some embodiments, L¹ is —(R)NSO₂—. In some embodiments, L¹ is —C(S)N(R)—. In some embodiments, L¹ is —(R)NC(S)—. or In some embodiments, L¹ is —(R)NC(S)N(R)—.

In some embodiments, L¹ is —CH₂—, —CH(CH₃)—, —NH—CH₂—, —NH—CH(CH₃)—, —C(O)—NH—, or —N(CH₃)—.

In some embodiments, L¹ is

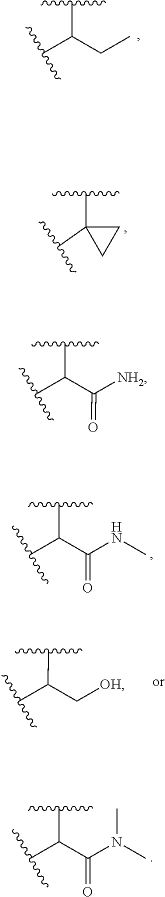

In some embodiments, L¹ is selected from those depicted in Table B, below.

As defined generally above, Ring A is a 4-, 5-, or 6-membered saturated or partially unsaturated monocyclic carbocyclic ring, phenyl, a 4-, 5-, or 6-membered saturated or partially unsaturated monocyclic heterocyclic ring having 1-2 heteroatoms independently selected from nitrogen, oxygen, or sulfur, or a 5-6 membered monocyclic heteroaromatic ring having 1, 2, 3, or 4 heteroatoms independently selected from nitrogen, oxygen, or sulfur, wherein Ring A is optionally substituted 1-2 times by halogen, —CN, —NO₂, or —C₁₋₆ aliphatic substituted 0-6 times by halogen, —CN, or —NO₂.

In some embodiments, Ring A is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur.

In some embodiments, Ring A is optionally substituted phenyl. In some embodiments, Ring A is optionally substituted 4-, 5-, or 6-membered saturated or partially unsaturated monocyclic carbocyclic ring. In some embodiments, Ring A is optionally substituted 4-, 5-, or 6-membered saturated or partially unsaturated monocyclic heterocyclic ring having 1-2 heteroatoms independently selected from nitrogen, oxygen, or sulfur. In some embodiments, Ring A is optionally substituted 5-6 membered monocyclic heteroaromatic ring having 1, 2, 3, or 4 heteroatoms independently selected from nitrogen, oxygen, or sulfur. In some embodiments, Ring A is optionally substituted 8-10 membered bicyclic aromatic ring. In some embodiments, Ring A is optionally substituted 8-10 membered bicyclic heteroaromatic ring having 1-5 heteroatoms independently selected from nitrogen, oxygen, or sulfur.

In some embodiments, Ring A is optionally substituted phenyl, a 6-membered monocyclic heteroaromatic ring having 1 or 2 nitrogen, or a 10-membered bicyclic heteroaromatic ring having 1-2 nitrogen.

In some embodiments, Ring A is optionally substituted

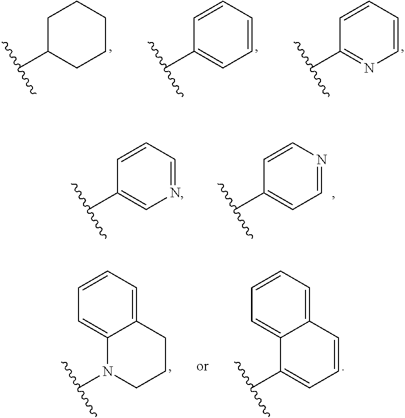

In some embodiments, Ring A is optionally substituted 1-2 times by -halogen, —CN, —NO₂, —C₁₋₆ aliphatic, or —O—C₁₋₆ aliphatic, wherein each of —C₁₋₆ aliphatic and —O—C₁₋₆ aliphatic is independently substituted 0-6 times by -halogen, —CN, or —NO₂. In some embodiments, Ring A is optionally substituted 1-2 times by halogen, —CN, —NO₂, —C₁₋₆ aliphatic, or —O—C₁₋₆ aliphatic, wherein each of —C₁₋₆ aliphatic and —O—C₁₋₆ aliphatic is independently substituted 0, 1, 2, 3, 4, 5, or 6 times by halogen, —CN, or —NO₂. In some embodiments, Ring A is optionally substituted 1-2 times by halogen, —C₁₋₆ aliphatic, or —O—C₁₋₆ aliphatic, wherein each of —C₁₋₆ aliphatic and —O—C₁₋₆ aliphatic is independently substituted 1, 2, 3, 4, 5, or 6 times by halogen.

In some embodiments, Ring A is a 4-, 5-, or 6-membered saturated or partially unsaturated monocyclic carbocyclic ring. In some embodiments, Ring A is cyclohexyl. In some embodiments, Ring A is phenyl. In some embodiments, Ring A is a 4-, 5-, or 6-membered saturated or partially unsaturated monocyclic heterocyclic ring having 1-2 heteroatoms independently selected from nitrogen, oxygen, or sulfur. In some embodiments, Ring A is a 5-6 membered monocyclic heteroaromatic ring having 1, 2, 3, or 4 heteroatoms independently selected from nitrogen, oxygen, or sulfur.

In some embodiments, Ring A is a 8-10 membered bicyclic aromatic ring. In some embodiments, Ring A is a 8-10 membered bicyclic heteroaromatic ring having 1-5 heteroatoms independently selected from nitrogen, oxygen, or sulfur.

In some embodiments, Ring A is optionally substituted 1-2 times by halogen, —CN, —NO₂, or —C₁₋₆ aliphatic substituted 0, 1, 2, 3, 4, 5, or 6 times by halogen, —CN, or —NO₂. In some embodiments, Ring A is optionally substituted 1-2 times by halogen, or —C₁₋₆ aliphatic substituted 0, 1, 2, 3, 4, 5, or 6 times by halogen.

In some embodiments, Ring A is selected from

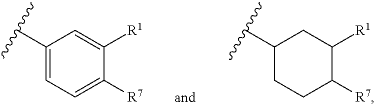

wherein each of R¹ and R⁷ is independently as described herein.

In some embodiments, Ring A is selected from

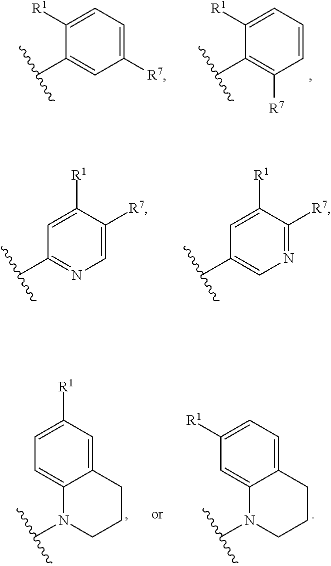

In some embodiments, R¹ is —H, -halogen, —CN, —NO₂, —C₁₋₆ aliphatic, or —O—C₁₋₆ aliphatic, wherein each of —C₁₋₆ aliphatic and —O—C₁₋₆ aliphatic is substituted 0, 1, 2, 3, 4, 5, or 6 times by -halogen, —CN, or —NO₂. In some embodiments, R¹ is unsubstituted —O—C₁₋₆ aliphatic. In some embodiments, R¹ is —OCH₃. In some embodiments, R¹ is —O—C₁₋₆ aliphatic substituted 1, 2, 3, 4, 5, or 6 times by -halogen. In some embodiments, R¹ is —O—C₁₋₃ aliphatic substituted 1, 2, 3, 4, 5, or 6 times by -halogen. In some embodiments, R¹ is —O—C₁₋₆ aliphatic substituted 1, 2, 3, 4, 5, or 6 times by —F.

In some embodiments, R¹ is —H, -halogen, —CN, —NO₂, or —C₁₋₆ aliphatic substituted 0, 1, 2, 3, 4, 5, or 6 times by -halogen, —CN, or —NO₂. In some embodiments, R¹ is —H. In some embodiments, R¹ is -halogen. In some embodiments, R¹ is —F. In some embodiments, R¹ is —Cl. In some embodiments, R¹ is —Br. In some embodiments, R¹ is —CN. In some embodiments, R¹ is —NO₂. In some embodiments, R¹ is unsubstituted —C₁₋₆ aliphatic. In some embodiments, R¹ is —CH₃. In some embodiments, R¹ is —C₁₋₆ aliphatic substituted 1, 2, 3, 4, 5, or 6 times by -halogen. In some embodiments, R¹ is —C₁₋₃ aliphatic substituted 1, 2, 3, 4, 5, or 6 times by -halogen. In some embodiments, R¹ is —C₁₋₆ aliphatic substituted 1, 2, 3, 4, 5, or 6 times by —F. In some embodiments, R¹ is —C₁₋₃ aliphatic substituted 1, 2, 3, 4, 5, or 6 times by —F. In some embodiments, R¹ is —CF₃. In some embodiments, R¹ is —C₁₋₆ aliphatic substituted 1, 2, 3, 4, 5, or 6 times by —CN. In some embodiments, R¹ is —C₁₋₆ aliphatic substituted 1, 2, 3, 4, 5, or 6 times by —NO₂.

In some embodiments, R¹ is phenyl. In some embodiments, R¹ is —C(CH₃)₃. In some embodiments, R¹ is —SCF₃. In some embodiments, R¹ is —S(O)₂CF₃. In some embodiments, R¹ is —N(CH₃)₂. In some embodiments, R¹ is —CHF₂. In some embodiments, R¹ is cyclopropyl. In some embodiments, R¹ is —CF₂CF₃. In some embodiments, R¹ is

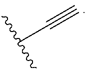 [SMILES: *C#C]

In some embodiments, R⁷ is —H, -halogen, —CN, —NO₂, —C₁₋₆ aliphatic, or —O—C₁₋₆ aliphatic, wherein each of —C₁₋₆ aliphatic and —O—C₁₋₆ aliphatic is substituted 0, 1, 2, 3, 4, 5, or 6 times by -halogen, —CN, or —NO₂. In some embodiments, R⁷ is unsubstituted —O—C₁₋₆ aliphatic. In some embodiments, R⁷ is —OCH₃. In some embodiments, R⁷ is —O—C₁₋₆ aliphatic substituted 1, 2, 3, 4, 5, or 6 times by -halogen. In some embodiments, R⁷ is —O—C₁₋₃ aliphatic substituted 1, 2, 3, 4, 5, or 6 times by -halogen. In some embodiments, R⁷ is —O—C₁₋₆ aliphatic substituted 1, 2, 3, 4, 5, or 6 times by —F.

In some embodiments, R⁷ is —H, -halogen, —CN, —NO₂, or —C₁₋₆ aliphatic substituted 0, 1, 2, 3, 4, 5, or 6 times by -halogen, —CN, or —NO₂. In some embodiments, R⁷ is —H. In some embodiments, R⁷ is -halogen. In some embodiments, R⁷ is —F. In some embodiments, R⁷ is —Cl. In some embodiments, R⁷ is —Br. In some embodiments, R⁷ is —CN. In some embodiments, R⁷ is —NO₂. In some embodiments, R⁷ is unsubstituted —C₁₋₆ aliphatic. In some embodiments, R¹ is —CH₃. In some embodiments, R⁷ is —C₁₋₆ aliphatic substituted 1, 2, 3, 4, 5, or 6 times by -halogen. In some embodiments, R⁷ is —C₁₋₃ aliphatic substituted 1, 2, 3, 4, 5, or 6 times by -halogen. In some embodiments, R⁷ is —C₁₋₆ aliphatic substituted 1, 2, 3, 4, 5, or 6 times by —F. In some embodiments, R⁷ is —C₁₋₃ aliphatic substituted 1, 2, 3, 4, 5, or 6 times by —F. In some embodiments, R⁷ is —CF₃. In some embodiments, R⁷ is —C₁₋₆ aliphatic substituted 1, 2, 3, 4, 5, or 6 times by —CN. In some embodiments, R⁷ is —C₁₋₆ aliphatic substituted 1, 2, 3, 4, 5, or 6 times by —NO₂.

In some embodiments, R⁷ is phenyl. In some embodiments, R⁷ is —C(CH₃)₃. In some embodiments, R⁷ is —SCF₃. In some embodiments, R⁷ is —S(O)₂CF₃. In some embodiments, R⁷ is —N(CH₃)₂. In some embodiments, R⁷ is —CHF₂. In some embodiments, R⁷ is cyclopropyl. In some embodiments, R⁷ is —CF₂CF₃. In some embodiments, R⁷ is.

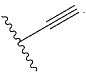 [SMILES: *C#C]

In some embodiments, Ring A is

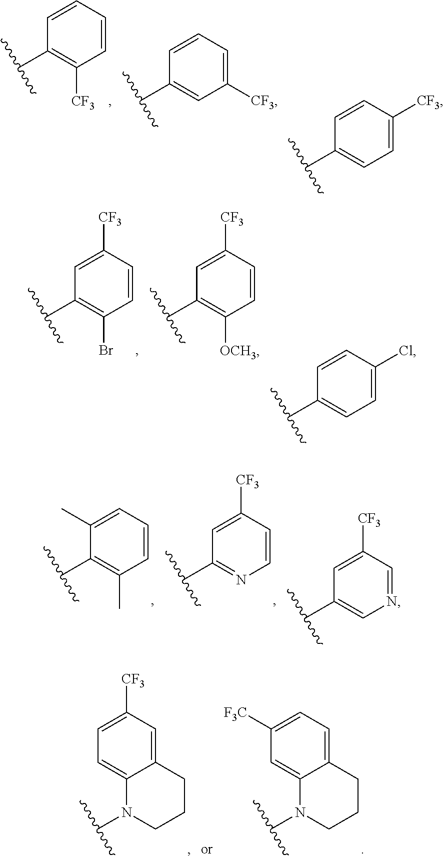

In some embodiments, Ring A is

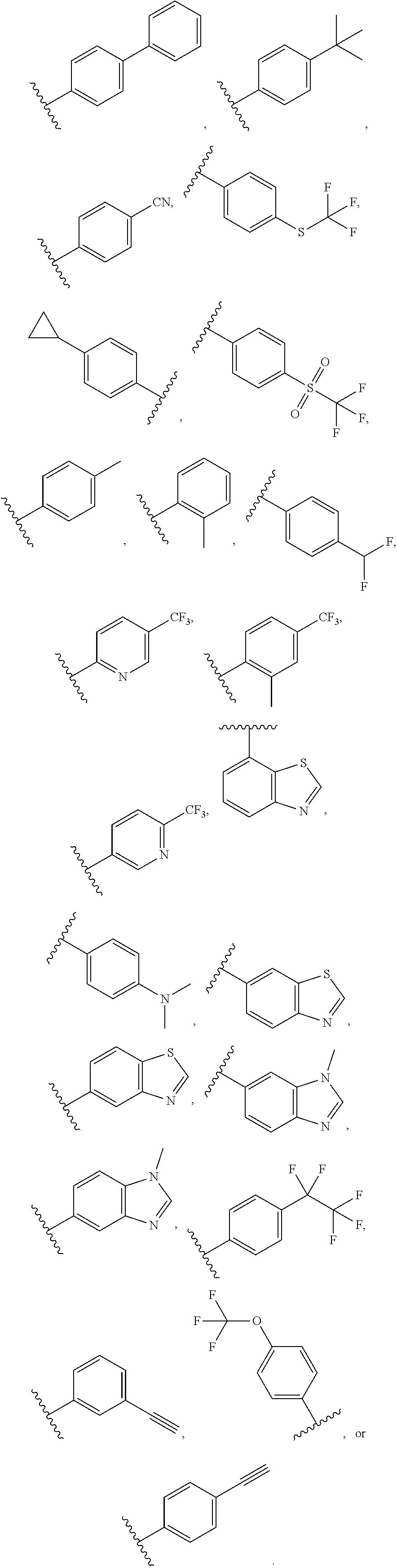

In some embodiments, Ring A is selected from those depicted in Table B, below.

As defined generally above, Ring B is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, a 8-10 membered bicyclic heteroaromatic ring having 1-5 heteroatoms independently selected from nitrogen, oxygen, or sulfur.

In some embodiments, Ring B is optionally substituted phenyl. In some embodiments, Ring B is optionally substituted 4-, 5-, or 6-membered saturated or partially unsaturated monocyclic carbocyclic ring. In some embodiments, Ring B is optionally substituted 4-, 5-, or 6-membered saturated or partially unsaturated monocyclic heterocyclic ring having 1-2 heteroatoms independently selected from nitrogen, oxygen, or sulfur. In some embodiments, Ring B is optionally substituted 5-6 membered monocyclic heteroaromatic ring having 1, 2, 3, or 4 heteroatoms independently selected from nitrogen, oxygen, or sulfur. In some embodiments, Ring B is optionally substituted 8-10 membered bicyclic aromatic ring. In some embodiments, Ring B is optionally substituted 8-10 membered bicyclic heteroaromatic ring having 1-5 heteroatoms independently selected from nitrogen, oxygen, or sulfur.

In some embodiments, Ring B is an optionally substituted 6-, 7-, 8-, 9-, or 10-membered bicyclic carbocyclic ring. In some embodiments, Ring B is an optionally substituted 6-, 7-, 8-, 9-, or 10-membered bicyclic heterocyclic ring having 1, 2, 3, 4, or 5 heteroatoms independently selected from nitrogen, oxygen, or sulfur. In some embodiments, Ring B is an optionally substituted 6-membered bicyclic heterocyclic ring having 1 nitrogen.

In some embodiments, Ring B is optionally substituted phenyl or a 6-membered monocyclic heteroaromatic ring having 1 or 2 nitrogen.

In some embodiments, Ring B is optionally substituted

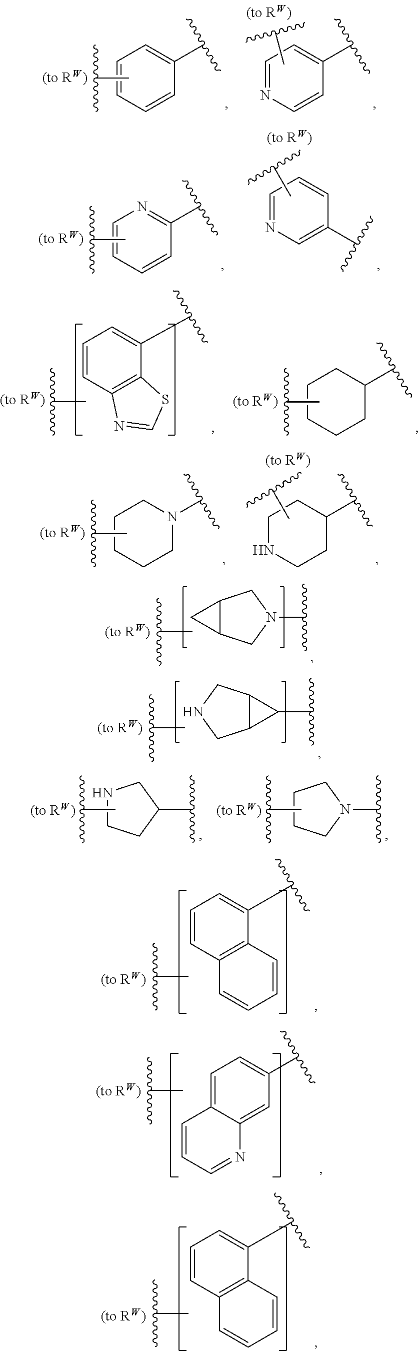

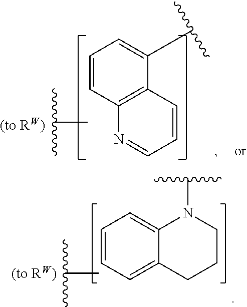

In some embodiments, Ring B is optionally substituted 1-4 times by halogen, —S(O)₂N(R)₂, —S(O)N(R)₂, —C(O)N(R)₂, —C(O)OR, —C₁₋₆ aliphatic, or —O—C₁₋₆ aliphatic, wherein each of —C₁₋₆ aliphatic and —O—C₁₋₆ aliphatic is independently substituted 0-6 times by halogen, —CN, or —NO₂.

In some embodiments, Ring B is optionally substituted 1-4 times by —F, —Cl, —Br—, —S(O)₂NHCH₃, —S(O)NHCH₃, —C(O)N(CH₃)₂, —C(O)NHCH₃, —C(O)OH, —C(O)OCH₃, —CH₃, —OCH₃, or —C(CH₃)₃.

In some embodiments, Ring B is

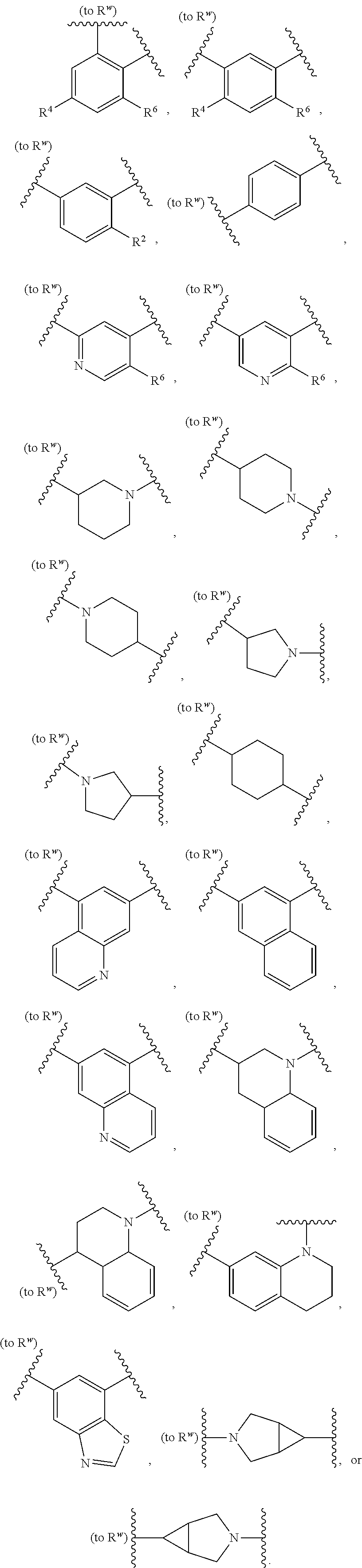

In some embodiments, Ring B is

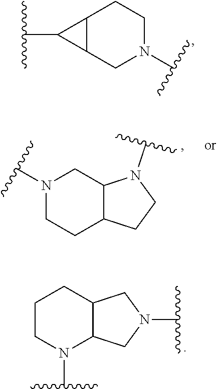 [SMILES: *C1C2CCN(*)CC12 ; *N1CCC2CCN(*)C2C1 ; *N1CC2CCCN(*)C2C1]

In some embodiments, Ring B is selected from those depicted in Table B, below.

As defined generally above, R² is —H, or a warhead group.

In some embodiments, R² is —H.

In some embodiments, R² is a warhead group. In some embodiments, R² is

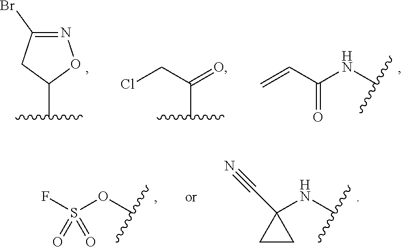

In some embodiments, R² is

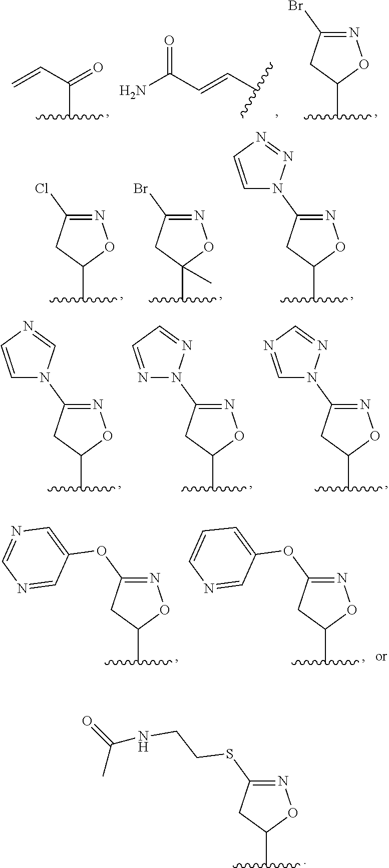

In some embodiments, R² is selected from those depicted in Table B, below.

As defined generally above, R³ is —H or a warhead group.

In some embodiments, R³ is —H.

In some embodiments, R³ is a warhead group. In some embodiments, R³ is

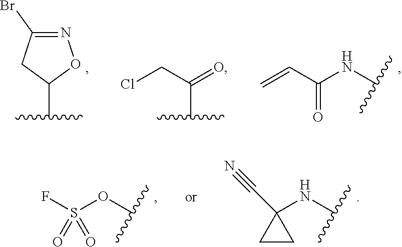

In some embodiments, R³ is

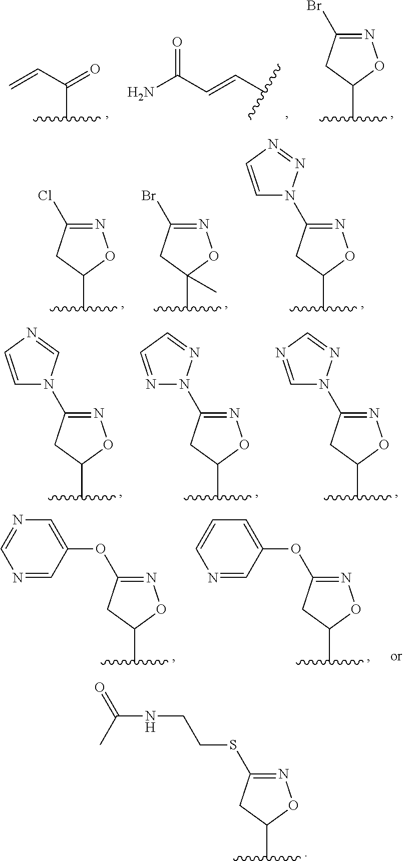

In some embodiments, R³ is selected from those depicted in Table B, below.

As defined generally above, R⁴ is —H, halogen, —S(O)₂N(R)₂, —S(O)N(R)₂, —C(O)N(R)₂, or a warhead group.

In some embodiments, R⁴ is —H, halogen, —S(O)₂N(R)₂, —S(O)N(R)₂, —C(O)N(R)₂, —C(O)OR, or a warhead group.

In some embodiments, R⁴ is —H.

In some embodiments, R⁴ is halogen. In some embodiments, R⁴ is —F. In some embodiments, R⁴ is —Cl. In some embodiments, R⁴ is —Br.

In some embodiments, R⁴ is —S(O)₂N(R)₂, —S(O)N(R)₂, or —C(O)N(R)₂. In some embodiments, R⁴ is —S(O)₂N(R)₂. In some embodiments, R⁴ is —S(O)N(R)₂. In some embodiments, R⁴ is —C(O)N(R)₂. In some embodiments, R⁴ is —S(O)₂NHCH₃.

In some embodiments, R⁴ is —S(O)NHCH₃, —C(O)N(CH₃)₂, —C(O)NHCH₃, —C(O)OH, or —C(O)OCH₃.

In some embodiments, R⁴ is a warhead group. In some embodiments, R⁴ is

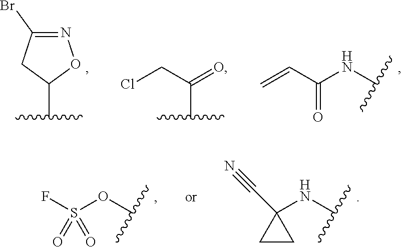

In some embodiments, R is

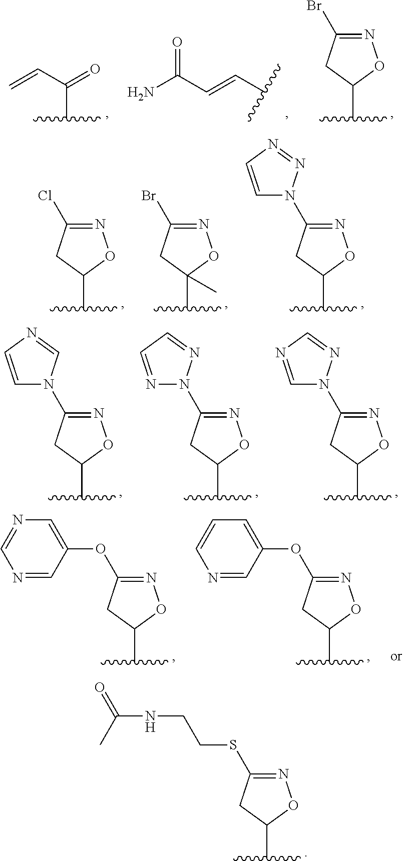

In some embodiments, R⁴ is selected from those depicted in Table B, below.

As defined generally above, R⁶ is —H or —C₁₋₆ aliphatic substituted 0, 1, 2, 3, 4, 5, or 6 times by -halogen, —CN, or —NO₂.

In some embodiments, R⁶ is —H, -halogen, —CN, —NO₂, —C₁₋₆ aliphatic, —OC₁₋₆ aliphatic, or a 4-, 5-, or 6-membered ring having 1, 2, 3, or 4 heteroatoms independently selected from nitrogen, oxygen, or sulfur optionally substituted 1-3 times by —C₁₋₆ aliphatic or —OC₁₋₆ aliphatic, wherein each of —C₁₋₆ aliphatic and —OC₁₋₆ aliphatic is independently substituted 0, 1, 2, 3, 4, 5, or 6 times by -halogen, —CN, or —NO₂.

In some embodiments, R⁶ is —H. In some embodiments, R⁶ is —F. In some embodiments, R⁶ is —Cl. In some embodiments, R⁶ is —Br. In some embodiments, R⁶ is —CN. In some embodiments, R⁶ is —NO₂.

In some embodiments, R⁶ is —C₁₋₆ aliphatic, substituted 0, 1, 2, 3, 4, 5, or 6 times by -halogen, —CN, or —NO₂. In some embodiments, R⁶ is unsubstituted —C₁₋₆ aliphatic. In some embodiments, R⁶ is —CH₃. In some embodiments, R⁶ is —C₁₋₆ aliphatic substituted 1, 2, 3, 4, 5, or 6 times by -halogen, —CN, or —NO₂. In some embodiments, R⁶ is —C₁₋₆ aliphatic substituted 1, 2, 3, 4, 5, or 6 times by —F. In some embodiments, R⁶ is —C₁₋₃ aliphatic substituted 1, 2, 3, 4, 5, or 6 times by —F. In some embodiments, R⁶ is —CF₃.

In some embodiments, R⁶ is —OC₁₋₆ aliphatic, substituted 0, 1, 2, 3, 4, 5, or 6 times by -halogen, —CN, or —NO₂. In some embodiments, R⁶ is unsubstituted —OC₁₋₆ aliphatic. In some embodiments, R⁶ is —OCH₃. In some embodiments, R⁶ is —OC₁₋₆ aliphatic substituted 1, 2, 3, 4, 5, or 6 times by -halogen, —CN, or —NO₂. In some embodiments, R⁶ is —OC₁₋₆ aliphatic substituted 1, 2, 3, 4, 5, or 6 times by —F. In some embodiments, R⁶ is —OC₁₋₃ aliphatic substituted 1, 2, 3, 4, 5, or 6 times by —F. In some embodiments, R⁶ is —OCF₃.

In some embodiments, R⁶ is a 4-, 5-, or 6-membered ring having 1, 2, 3, or 4 heteroatoms independently selected from nitrogen, oxygen, or sulfur optionally substituted 1-3 times by —C₁₋₆ aliphatic or —OC₁₋₆ aliphatic, wherein each of —C₁₋₆ aliphatic and —OC₁₋₆ aliphatic is independently substituted 0, 1, 2, 3, 4, 5, or 6 times by -halogen, —CN, or —NO₂. In some embodiments, R⁶ is a 5-membered ring having 1, 2, 3, or 4 nitrogen optionally substituted 1-3 times by —C₁₋₆ aliphatic. In some embodiments, R⁶ is

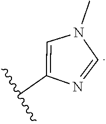 [SMILES: *c1cn(C)cn1]

In some embodiments, R⁶ is selected from those depicted in Table B, below.

As defined generally above, R^{w} is a warhead group; wherein when R^{w} is a saturated or partially unsaturated monocyclic carbocyclic or heterocyclic ring, it optionally forms a spiro bicyclic ring with Ring B.

In some embodiments, R^{w} is a warhead group.

In some embodiments, R^{w} is

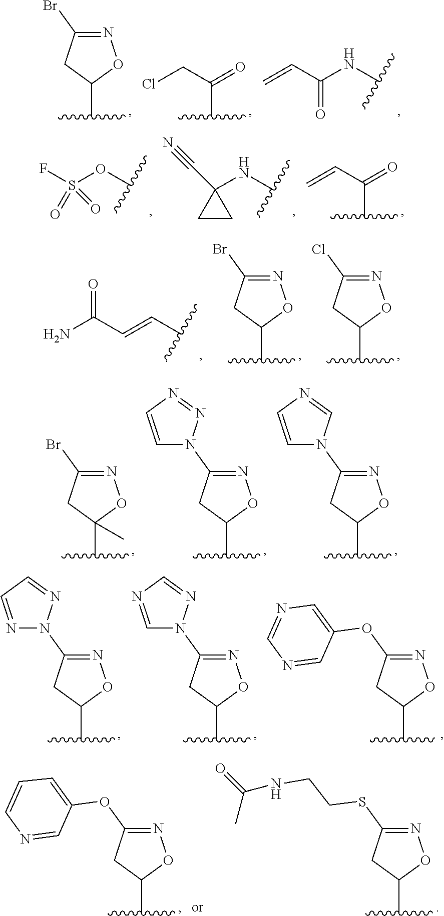

In some embodiments, wherein R^{w} is a saturated or partially unsaturated monocyclic carbocyclic or heterocyclic ring, R^{w} forms a spiro bicyclic ring with Ring B. In some embodiments, wherein R^{w} is a saturated or partially unsaturated 4-, 5-, or 6-membered carbocyclic or heterocyclic ring, R^{w} forms a spiro bicyclic ring with Ring B. In some embodiments, wherein R^{w} is optionally substituted

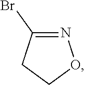

it forms a spiro bicyclic ring with Ring B. In some embodiments, wherein R^{w} is optionally substituted

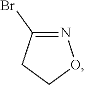

it forms a spiro bicyclic ring with Ring B, for example,

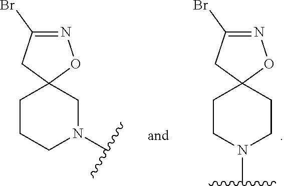 [SMILES: *N1CCCC2(CC(Br)=NO2)C1 ; *N1CCC2(CC1)CC(Br)=NO2]

In some embodiments, R^{w} is selected from those depicted in Table B, below.

As defined generally above, R is independently —H or optionally substituted —C₁₋₆ aliphatic.

In some embodiments, R is —H.

In some embodiments, R is optionally substituted —C₁₋₆ aliphatic. In some embodiments, R is unsubstituted —C₁₋₆ aliphatic. In some embodiments, R is —C₁₋₆ aliphatic substituted 1, 2, 3, 4, 5, or 6 times by -halogen, —CN, or —NO₂. In some embodiments, R is —C₁₋₆ aliphatic substituted 1, 2, 3, 4, 5, or 6 times by —F. In some embodiments, R is —C₁₋₃ aliphatic substituted 1, 2, 3, 4, 5, or 6 times by —F. In some embodiments, R is —CF₃.

In some embodiments, R is —CH₃, —C(CH₃)₃, —CHF₂, cyclopropyl, —CF₂CF₃, or

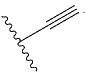 [SMILES: *C#C]

In some embodiments, R is selected from those depicted in Table B, below.

A “warhead group,” as used herein, is capable of covalently binding to an amino acid residue (such as cysteine, lysine, histidine, or other residues capable of being covalently modified) present in the binding pocket of a target protein, for example, TEAD, thereby irreversibly inhibiting the protein. In some embodiments, a warhead group is as defined and described in embodiments in WO 2020/243423, the content of which is herein incorporated by reference in its entirety.

In some embodiments, a TEAD inhibitor is a compound of Formula B-2:

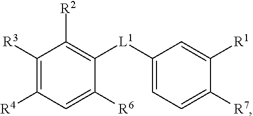

or a pharmaceutically acceptable salt thereof, wherein each of R¹, R², R³, R⁴, R⁶, R⁷, and L¹ is independently as defined and described in embodiments in the section of TEAD Inhibitors of Formulae B, and B-1 to B-34.

In some embodiments, a TEAD inhibitor is a compound of formula B-2, or a pharmaceutically acceptable salt thereof, wherein:
(a):
   L¹ is —NH—;
   R¹ is —C₁₋₆ aliphatic substituted 1, 2, 3, 4, 5, or 6 times by halogen;
   R² is a warhead group;
   R³ is —H;
   R⁴ is —H, —S(O)₂N(R)₂; —S(O)N(R)₂, or —C(O)N(R)₂, each R independently is selected from —H and optionally substituted —C₁₋₆ aliphatic;
   R⁶ is —H or —C₁₋₆ aliphatic; and
   R⁷ is —H; or
(b):
   L¹ is —NH—;
   R¹ is —C₁₋₆ aliphatic substituted 1, 2, 3, 4, 5, or 6 times by halogen;
   R² is an optionally substituted 5-membered aromatic ring having 1, 2, 3, or 4 nitrogen;
   R³ is —H;
   R⁴ is a warhead group;
   R⁶ is —H or —C₁₋₆ aliphatic; and
   R⁷ is —H; or
(c):
   L¹ is —O—;
   R¹ is —H, or —C₁₋₆ aliphatic substituted 1, 2, 3, 4, 5, or 6 times by halogen;
   R² is —H;
   R³ is a warhead group;
   R⁴ is —H;
   R⁶ is —H or —C₁₋₆ aliphatic;
   R⁷ is —H; or
(d):
   L¹ is —O—;
   R¹ is —H, or —C₁₋₆ aliphatic substituted 1, 2, 3, 4, 5, or 6 times by halogen;
   R² is —H;
   R³ is a warhead group;
   R⁴ is —H;
   R⁶ is —H;
   R⁷ is —H or halogen; or
(e):
   L¹ is —O—;
   R¹ is —H, or —C₁₋₆ aliphatic substituted 1, 2, 3, 4, 5, or 6 times by halogen;
   R² is —H;
   R³ is a warhead group;
   R⁴ is —H;
   R⁶ is —H or —C₁₋₆ aliphatic; and
   R⁷ is —H or halogen; or
(f):
   L¹ is —NH—;
   R¹ is —H, or —C₁₋₆ aliphatic substituted 1, 2, 3, 4, 5, or 6 times by halogen;
   R² is —H;
   R³ is a warhead group;
   R⁴ is —H;
   R⁶ is —H or —C₁₋₆ aliphatic;
   R⁷ is —H or halogen; or
(g):
   L¹ is —NH—;
   R¹ is —C₁₋₆ aliphatic substituted 1, 2, 3, 4, 5, or 6 times by halogen;
   each of R² and R⁴ independently is a warhead group;
   R³ is —H;
   R⁶ is —H or —C₁₋₆ aliphatic; and
   R⁷ is —H or halogen.

In some embodiments, a TEAD inhibitor is a compound of Formula B-3:

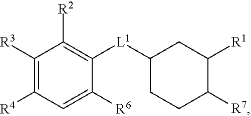

or a pharmaceutically acceptable salt thereof, wherein each of R¹, R², R³, R⁴, R⁶, R⁷, and L¹ is independently as defined and described in embodiments in the section of TEAD Inhibitors of Formulae B, and B-1 to B-34.

In some embodiments, a TEAD inhibitor is a compound of formula B-3, or a pharmaceutically acceptable salt thereof, wherein:
L¹ is —NH—;
R¹ is —C₁₋₆ aliphatic substituted 1, 2, 3, 4, 5, or 6 times by halogen;
R² is a warhead group;
R³ is —H;
R⁴ is —S(O)₂N(R)₂, —S(O)N(R)₂, or —C(O)N(R)₂, each R independently is selected from —H and optionally substituted —C₁₋₆ aliphatic;
R⁶ is —H or —C₁₋₆ aliphatic; and
R⁷ is —H or halogen.

In some embodiments, a TEAD inhibitor is a compound selected from the following:
i. Formula (B-4):

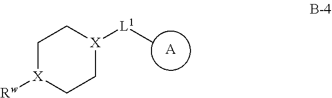

wherein each of X is independently C or N; and each of Ring A, R^{w}, and L¹ is independently as defined and described in embodiments in the section of TEAD Inhibitors of Formulae B, and B-1 to B-34;
ii. Formula (B-5) or (B-6):

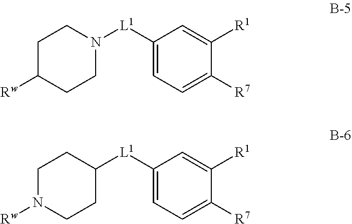

wherein each of R¹, R⁷, R^{w}, and L¹ is independently as defined and described in embodiments in the section of TEAD Inhibitors of Formulae B, and B-1 to B-34;
iii. Formula (B-7):

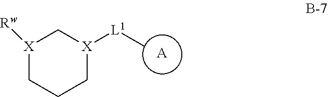

wherein each of X is independently C or N; and each of Ring A, R^{w}, and L¹ is independently as defined and described in embodiments in the section of TEAD Inhibitors of Formulae B, and B-1 to B-34;
iv. Formula (B-8) or (B-9):

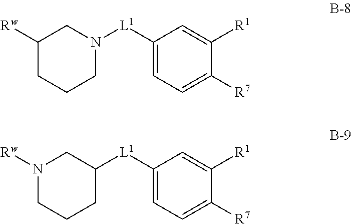

wherein each of R¹, R⁷, R^{w}, and L¹ is independently as defined and described in embodiments in the section of TEAD Inhibitors of Formulae B, and B-1 to B-34;
v. Formula (B-10):

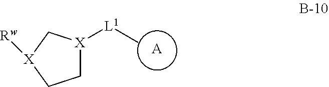

wherein each of X is independently C or N; and each of Ring A, R^{w}, and L¹ is independently as defined and described in embodiments in the section of TEAD Inhibitors of Formulae B, and B-1 to B-34;
vi. Formula (B-11) or (B-12):

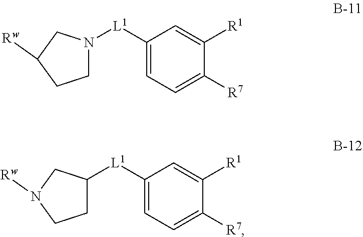

wherein each of R¹, R⁷, R^{w}, and L¹ is independently as defined and described in embodiments in the section of TEAD Inhibitors of Formulae B, and B-1 to B-34;
vii. Formula (B-13):

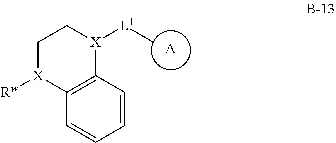

wherein each of X is independently C or N; and each of Ring A, R^{w}, and L¹ is independently as defined and described in embodiments in the section of TEAD Inhibitors of Formulae B, and B-1 to B-34;
viii. Formula (B-14) or (B-15):

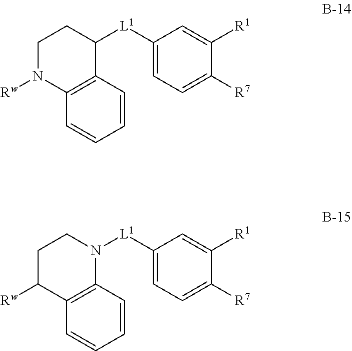

wherein each of R¹, R⁷, R^{w}, and L¹ is independently as defined and described in embodiments in the section of TEAD Inhibitors of Formulae B, and B-1 to B-34;
ix. Formula (B-16):

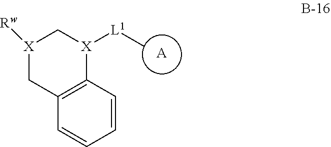

wherein each of X is independently C or N; and each of Ring A, R^{w}, and L¹ is independently as defined and described in embodiments in the section of TEAD Inhibitors of Formulae B, and B-1 to B-34; or
x. Formula (B-17) or (B-18):

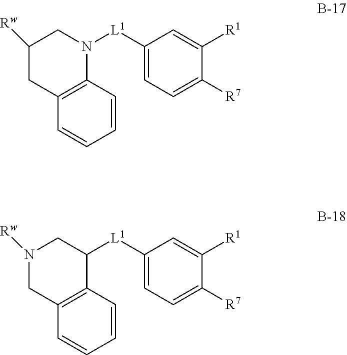

wherein each of R¹, R⁷, R^{w}, and L¹ is independently as defined and described in embodiments in the section of TEAD Inhibitors of Formulae B, and B-1 to B-34.

In some embodiments, a TEAD Inhibitor is a compound selected from Formulae B-4 to B-18, wherein L¹ is —CH₂—, —O—, —CH(CH₃)—, —NH—, —C(O)—, or —NH—CH₂—; R¹ is —H or —C₁₋₆ aliphatic substituted 1, 2, 3, 4, 5, or 6 times by halogen; R^{w} is a warhead group; and R⁷ is —H or —C₁₋₆ aliphatic substituted 1, 2, 3, 4, 5, or 6 times by halogen.

In some embodiments, a TEAD Inhibitor is a compound of Formula B, or a pharmaceutically acceptable salt thereof, wherein Ring A is phenyl, a 6-membered monocyclic heteroaromatic ring having 1 or 2 nitrogen, or a 10-membered bicyclic heteroaromatic ring having 1-2 nitrogen; Ring B is phenyl or a 6-membered monocyclic heteroaromatic ring having 1 or 2 nitrogen; and each of R^{w} and L¹ is independently as defined and described in embodiments in the section of TEAD Inhibitors of Formulae B, and B-1 to B-34.

In some embodiments, a TEAD Inhibitor is a compound selected from the following:
i. Formula (B-19):

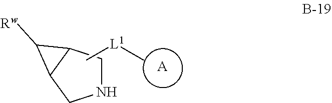

wherein each of Ring A, R^{w}, and L¹ is independently as defined and described in embodiments in the section of TEAD Inhibitors of Formulae B, and B-1 to B-34;
ii. Formula (B-20) or (B-21):

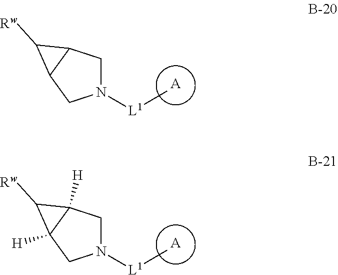

wherein each of Ring A, R^{w}, and L¹ is independently as defined and described in embodiments in the section of TEAD Inhibitors of Formulae B, and B-1 to B-34;
iii. Formula (B-22):

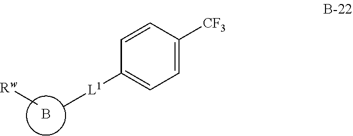

wherein each of Ring B, R^{w}, and L¹ is independently as defined and described in embodiments in the section of TEAD Inhibitors of Formulae B, and B-1 to B-34; optionally, L¹ is not —NH—C(O)— or —O—CH₂—;
iv. Formula (B-23):

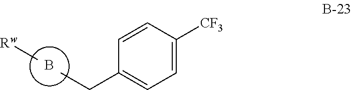

wherein each of Ring B and R^{w} is independently as defined and described in embodiments in the section of TEAD Inhibitors of Formulae B, and B-1 to B-34;
v. Formula (B-24):

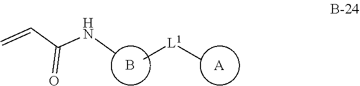

wherein each of Ring A, Ring B, and L¹ is independently as defined and described in embodiments in the section of TEAD Inhibitors of Formulae B, and B-1 to B-34, with the proviso that Ring B is not

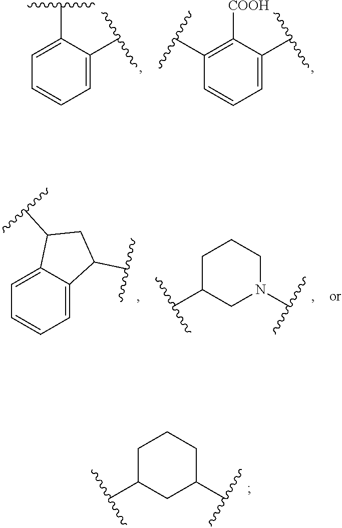 [SMILES: *c1ccccc1* ; *c1cccc(*)c1C(=O)O ; *C1CC(*)c2ccccc21 ; *C1CCCN(*)C1 ; *C1CCCC(*)C1]

optionally, Ring B is an optionally substituted 6-, 7-, 8-, 9-, or 10-membered bicyclic heterocyclic ring having 1, 2, 3, 4, or 5 heteroatoms independently selected from nitrogen, oxygen, or sulfur; further optionally, Ring B is an optionally substituted 6-membered bicyclic heterocyclic ring having 1 nitrogen;
vi. Formula (B-25):

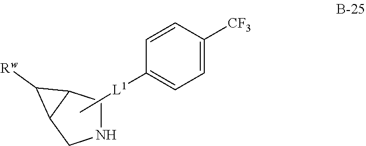

wherein each of R^{w} and L¹ is independently as defined and described in embodiments in the section of TEAD Inhibitors of Formulae B, and B-1 to B-34;
vii. Formula (B-26) or (B-27):

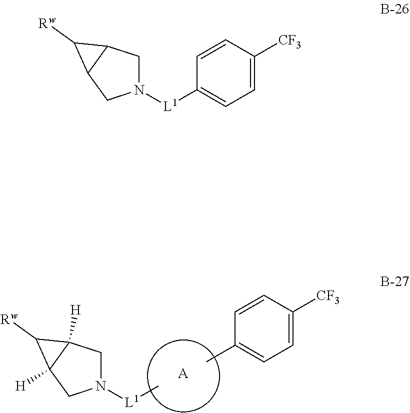

wherein each of R^{w} and L¹ is independently as defined and described in embodiments in the section of TEAD Inhibitors of Formulae B, and B-1 to B-34;
viii. Formula (B-28):

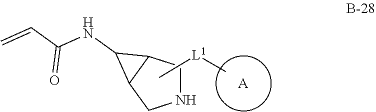

wherein each of Ring A and L¹ is independently as defined and described in embodiments in the section of TEAD Inhibitors of Formulae B, and B-1 to B-34;
ix. Formula (B-29) or (B-30):

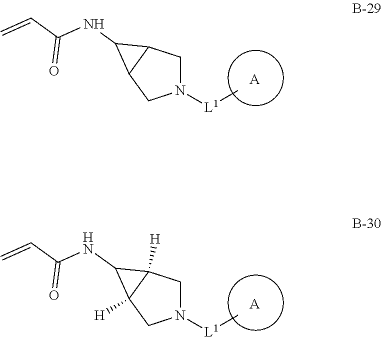

wherein each of Ring A and L¹ is independently as defined and described in embodiments in the section of TEAD Inhibitors of Formulae B, and B-1 to B-34;
x. Formula (B-31):

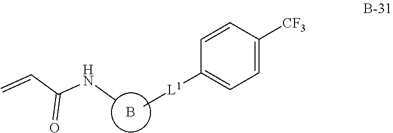

wherein each of Ring B and L¹ is independently as defined and described in embodiments in the section of TEAD Inhibitors of Formulae B, and B-1 to B-34; optionally, L¹ is —CH₂—;
xi. Formula (B-32):

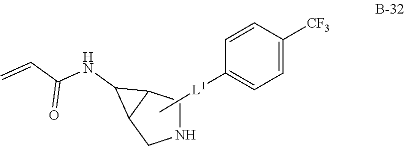

wherein L¹ is as defined and described in embodiments in the section of TEAD Inhibitors of Formulae B, and B-1 to B-34; or
xii. Formula (B-33) or (B-34):

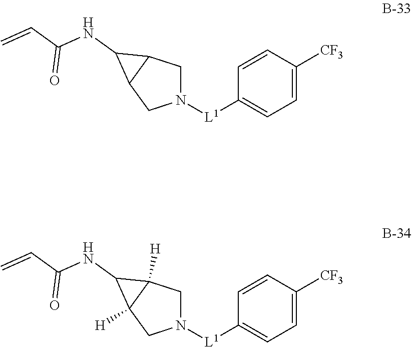

wherein L¹ is as defined and described in embodiments in the section of TEAD Inhibitors of Formulae B, and B-1 to B-34.

In some embodiments, a TEAD inhibitor is selected from those listed in Table B, or a pharmaceutically acceptable salt thereof.

TABLE B
Exemplified TEAD Inhibitors

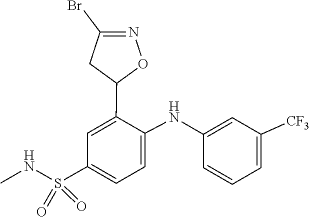 [SMILES: CNS(=O)(=O)c1ccc(Nc2cccc(C(F)(F)F)c2)c(C2CC(Br)=NO2)c1]

T-B-1

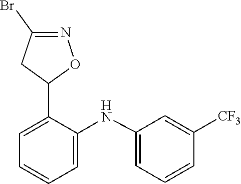 [SMILES: FC(F)(F)c1cccc(Nc2ccccc2C2CC(Br)=NO2)c1]

T-B-2

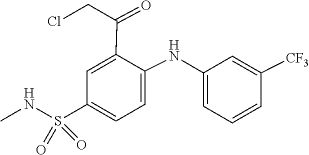 [SMILES: CNS(=O)(=O)c1ccc(Nc2cccc(C(F)(F)F)c2)c(C(=O)CCl)c1]

T-B-3

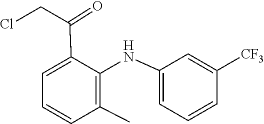 [SMILES: Cc1cccc(C(=O)CCl)c1Nc1cccc(C(F)(F)F)c1]

T-B-4

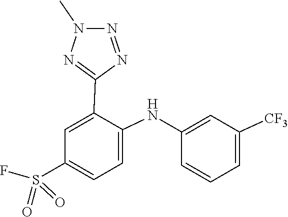 [SMILES: Cn1nnc(-c2cc(S(=O)(=O)F)ccc2Nc2cccc(C(F)(F)F)c2)n1]

T-B-5

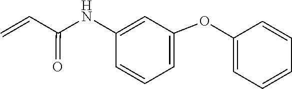 [SMILES: C=CC(=O)Nc1cccc(Oc2ccccc2)c1]

T-B-6

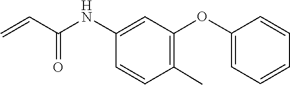 [SMILES: C=CC(=O)Nc1ccc(C)c(Oc2ccccc2)c1]

T-B-7

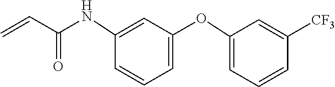 [SMILES: C=CC(=O)Nc1cccc(Oc2cccc(C(F)(F)F)c2)c1]

T-B-8

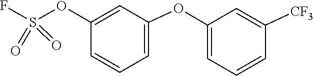 [SMILES: O=S(=O)(F)Oc1cccc(Oc2cccc(C(F)(F)F)c2)c1]

T-B-9

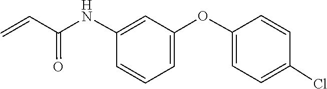 [SMILES: C=CC(=O)Nc1cccc(Oc2ccc(Cl)cc2)c1]

T-B-10

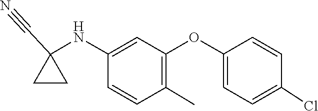 [SMILES: Cc1ccc(NC2(C#N)CC2)cc1Oc1ccc(Cl)cc1]

T-B-11

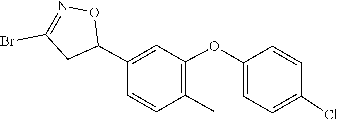 [SMILES: Cc1ccc(C2CC(Br)=NO2)cc1Oc1ccc(Cl)cc1]

T-B-12

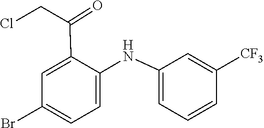 [SMILES: O=C(CCl)c1cc(Br)ccc1Nc1cccc(C(F)(F)F)c1]

T-B-13

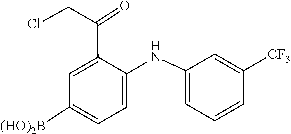 [SMILES: O=C(CCl)c1cc(B(O)O)ccc1Nc1cccc(C(F)(F)F)c1]

T-B-14

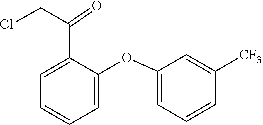 [SMILES: O=C(CCl)c1ccccc1Oc1cccc(C(F)(F)F)c1]

T-B-15

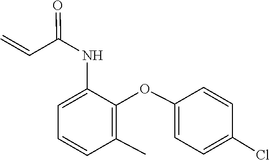 [SMILES: C=CC(=O)Nc1cccc(C)c1Oc1ccc(Cl)cc1]

T-B-16

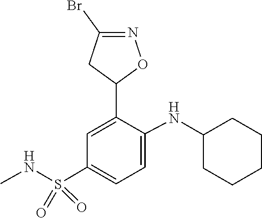 [SMILES: CNS(=O)(=O)c1ccc(NC2CCCCC2)c(C2CC(Br)=NO2)c1]

T-B-17

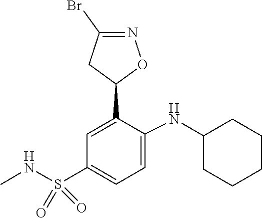 [SMILES: CNS(=O)(=O)c1ccc(NC2CCCCC2)c([C@H]2CC(Br)=NO2)c1]

T-B-18

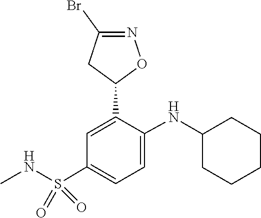 [SMILES: CNS(=O)(=O)c1ccc(NC2CCCCC2)c([C@@H]2CC(Br)=NO2)c1]

T-B-19

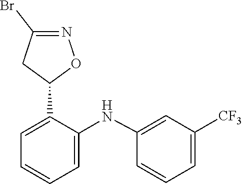 [SMILES: FC(F)(F)c1cccc(Nc2ccccc2[C@@H]2CC(Br)=NO2)c1]

T-B-20

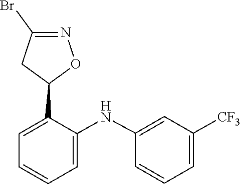 [SMILES: FC(F)(F)c1cccc(Nc2ccccc2[C@H]2CC(Br)=NO2)c1]

T-B-21

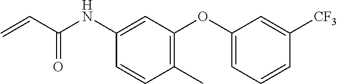 [SMILES: C=CC(=O)Nc1ccc(C)c(Oc2cccc(C(F)(F)F)c2)c1]

T-B-22

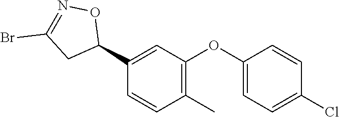 [SMILES: Cc1ccc([C@H]2CC(Br)=NO2)cc1Oc1ccc(Cl)cc1]

T-B-23

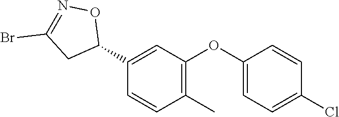 [SMILES: Cc1ccc([C@@H]2CC(Br)=NO2)cc1Oc1ccc(Cl)cc1]

T-B-24

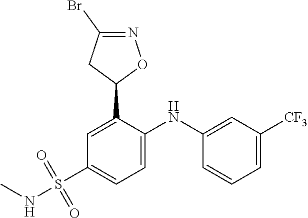 [SMILES: CNS(=O)(=O)c1ccc(Nc2cccc(C(F)(F)F)c2)c([C@H]2CC(Br)=NO2)c1]

T-B-25

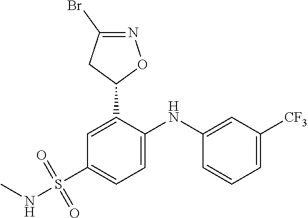 [SMILES: CNS(=O)(=O)c1ccc(Nc2cccc(C(F)(F)F)c2)c([C@@H]2CC(Br)=NO2)c1]

T-B-26

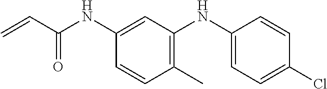 [SMILES: C=CC(=O)Nc1ccc(C)c(Nc2ccc(Cl)cc2)c1]

T-B-27

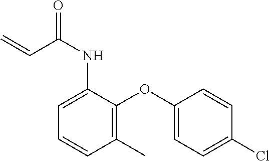 [SMILES: C=CC(=O)Nc1cccc(C)c1Oc1ccc(Cl)cc1]

T-B-28

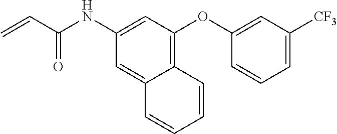 [SMILES: C=CC(=O)Nc1cc(Oc2cccc(C(F)(F)F)c2)c2ccccc2c1]

T-B-29

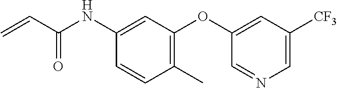 [SMILES: C=CC(=O)Nc1ccc(C)c(Oc2cncc(C(F)(F)F)c2)c1]

T-B-30

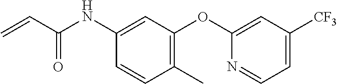 [SMILES: C=CC(=O)Nc1ccc(C)c(Oc2cc(C(F)(F)F)ccn2)c1]

T-B-31

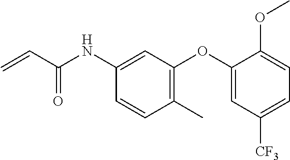 [SMILES: C=CC(=O)Nc1ccc(C)c(Oc2cc(C(F)(F)F)ccc2OC)c1]

T-B-32

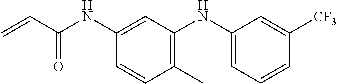 [SMILES: C=CC(=O)Nc1ccc(C)c(Nc2cccc(C(F)(F)F)c2)c1]

T-B-33

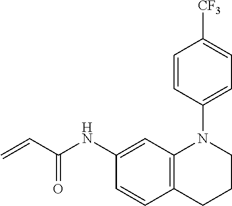 [SMILES: C=CC(=O)Nc1ccc2c(c1)N(c1ccc(C(F)(F)F)cc1)CCC2]

T-B-34

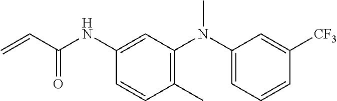 [SMILES: C=CC(=O)Nc1ccc(C)c(N(C)c2cccc(C(F)(F)F)c2)c1]

T-B-35

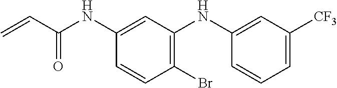 [SMILES: C=CC(=O)Nc1ccc(Br)c(Nc2cccc(C(F)(F)F)c2)c1]

T-B-36

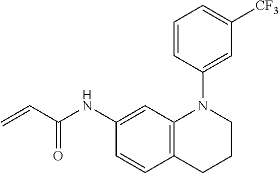 [SMILES: C=CC(=O)Nc1ccc2c(c1)N(c1cccc(C(F)(F)F)c1)CCC2]

T-B-37

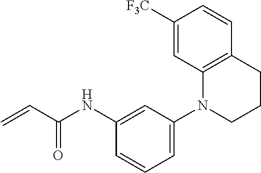 [SMILES: C=CC(=O)Nc1cccc(N2CCCc3ccc(C(F)(F)F)cc32)c1]

T-B-38

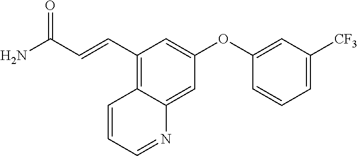 [SMILES: NC(=O)/C=C/c1cc(Oc2cccc(C(F)(F)F)c2)cc2ncccc12]

T-B-39

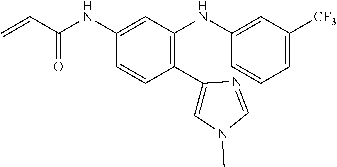 [SMILES: C=CC(=O)Nc1ccc(-c2cn(C)cn2)c(Nc2cccc(C(F)(F)F)c2)c1]

T-B-40

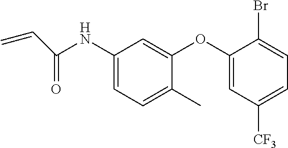 [SMILES: C=CC(=O)Nc1ccc(C)c(Oc2cc(C(F)(F)F)ccc2Br)c1]

T-B-41

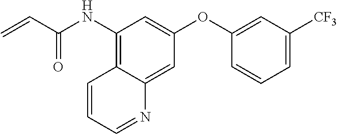 [SMILES: C=CC(=O)Nc1cc(Oc2cccc(C(F)(F)F)c2)cc2ncccc12]

T-B-42

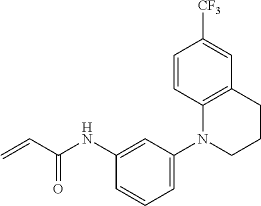 [SMILES: C=CC(=O)Nc1cccc(N2CCCc3cc(C(F)(F)F)ccc32)c1]

T-B-43

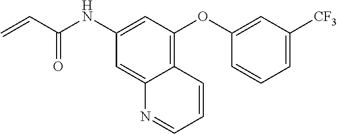 [SMILES: C=CC(=O)Nc1cc(Oc2cccc(C(F)(F)F)c2)c2cccnc2c1]

T-B-44

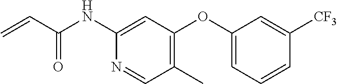 [SMILES: C=CC(=O)Nc1cc(Oc2cccc(C(F)(F)F)c2)c(C)cn1]

T-B-45

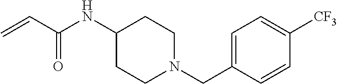 [SMILES: C=CC(=O)NC1CCN(Cc2ccc(C(F)(F)F)cc2)CC1]

T-B-46

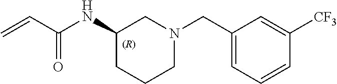

T-B-47

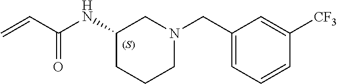

T-B-48

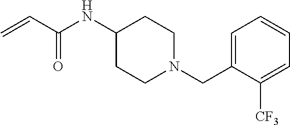 [SMILES: C=CC(=O)NC1CCN(Cc2ccccc2C(F)(F)F)CC1]

T-B-49

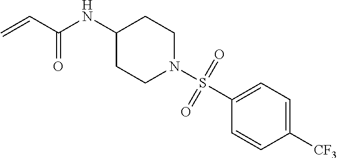 [SMILES: C=CC(=O)NC1CCN(S(=O)(=O)c2ccc(C(F)(F)F)cc2)CC1]

T-B-50

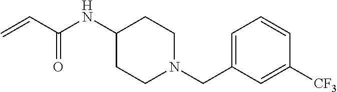 [SMILES: C=CC(=O)NC1CCN(Cc2cccc(C(F)(F)F)c2)CC1]

T-B-51

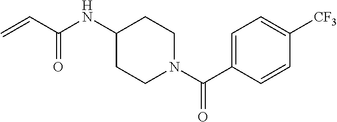 [SMILES: C=CC(=O)NC1CCN(C(=O)c2ccc(C(F)(F)F)cc2)CC1]

T-B-52

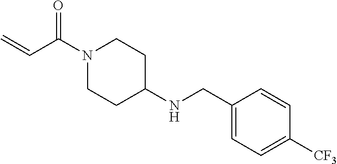 [SMILES: C=CC(=O)N1CCC(NCc2ccc(C(F)(F)F)cc2)CC1]

T-B-53

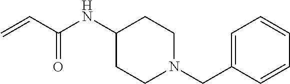 [SMILES: C=CC(=O)NC1CCN(Cc2ccccc2)CC1]

T-B-54

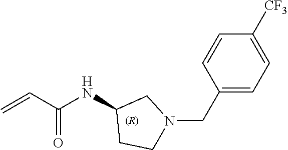

T-B-55

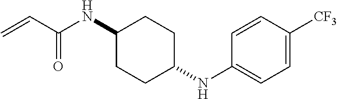

T-B-56

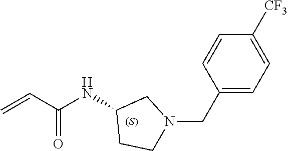

T-B-57

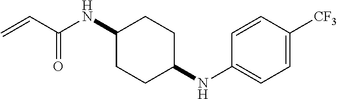

T-B-58

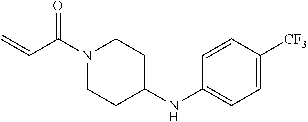 [SMILES: C=CC(=O)N1CCC(Nc2ccc(C(F)(F)F)cc2)CC1]

T-B-59

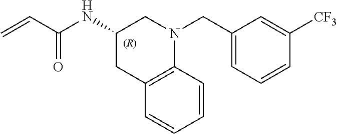

T-B-60

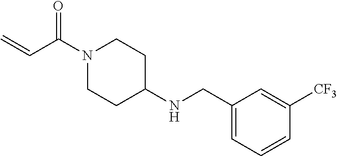 [SMILES: C=CC(=O)N1CCC(NCc2cccc(C(F)(F)F)c2)CC1]

T-B-61

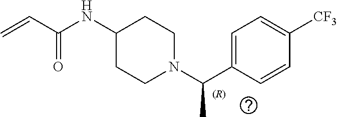

T-B-62

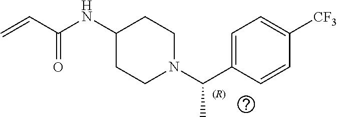

T-B-63

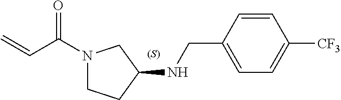

T-B-64

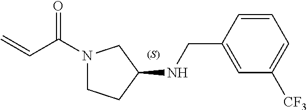

T-B-65

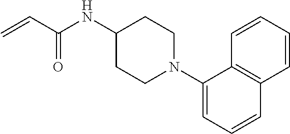 [SMILES: C=CC(=O)NC1CCN(c2cccc3ccccc23)CC1]

T-B-66

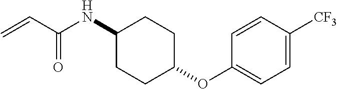

T-B-67

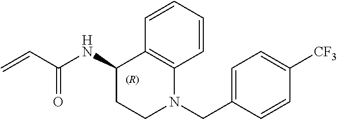

T-B-68

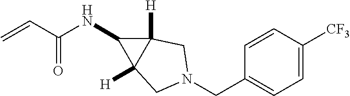 [SMILES: C=CC(=O)N[C@H]1[C@@H]2CN(Cc3ccc(C(F)(F)F)cc3)C[C@@H]21]

T-B-69

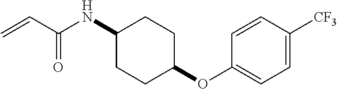

T-B-70

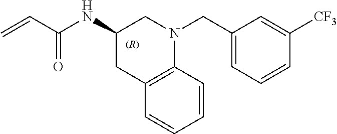

T-B-71

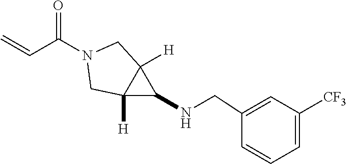

T-B-72

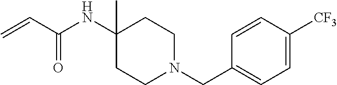 [SMILES: C=CC(=O)NC1(C)CCN(Cc2ccc(C(F)(F)F)cc2)CC1]

T-B-73

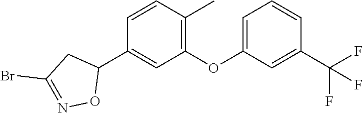 [SMILES: Cc1ccc(C2CC(Br)=NO2)cc1Oc1cccc(C(F)(F)F)c1]

T-B-74

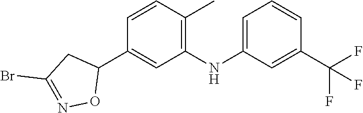 [SMILES: Cc1ccc(C2CC(Br)=NO2)cc1Nc1cccc(C(F)(F)F)c1]

T-B-75

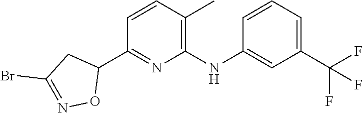 [SMILES: Cc1ccc(C2CC(Br)=NO2)nc1Nc1cccc(C(F)(F)F)c1]

T-B-76

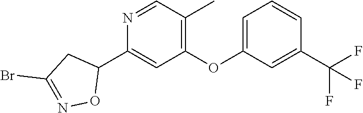 [SMILES: Cc1cnc(C2CC(Br)=NO2)cc1Oc1cccc(C(F)(F)F)c1]

T-B-77

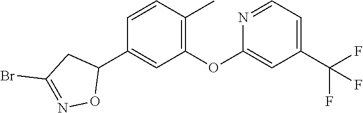 [SMILES: Cc1ccc(C2CC(Br)=NO2)cc1Oc1cc(C(F)(F)F)ccn1]

T-B-78

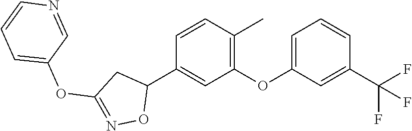 [SMILES: Cc1ccc(C2CC(Oc3cccnc3)=NO2)cc1Oc1cccc(C(F)(F)F)c1]

T-B-79

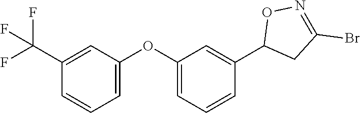 [SMILES: FC(F)(F)c1cccc(Oc2cccc(C3CC(Br)=NO3)c2)c1]

T-B-80

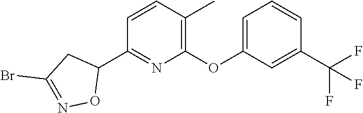 [SMILES: Cc1ccc(C2CC(Br)=NO2)nc1Oc1cccc(C(F)(F)F)c1]

T-B-81

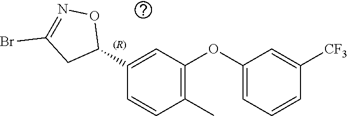

T-B-82

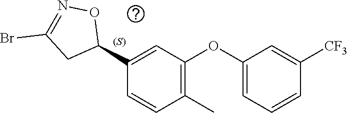

T-B-83

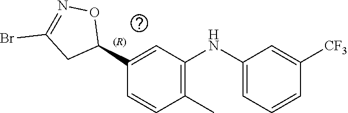

T-B-84

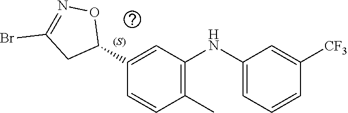

T-B-85

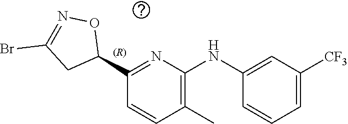

T-B-86

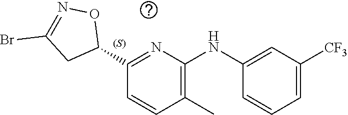

T-B-87

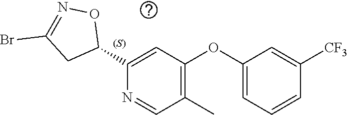

T-B-88

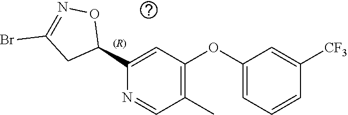

T-B-89

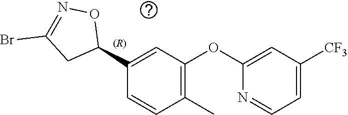

T-B-90

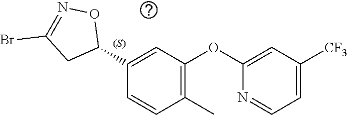

T-B-91

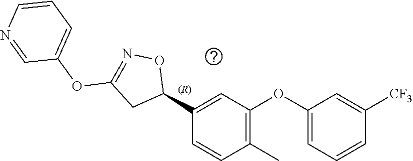

T-B-92

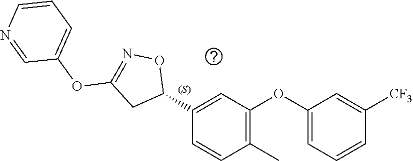

T-B-93

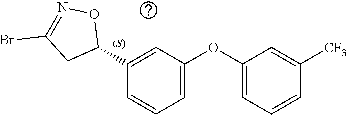

T-B-94

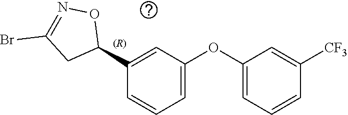

T-B-95

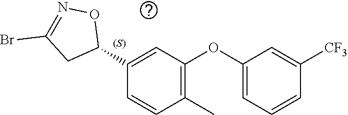

T-B-96

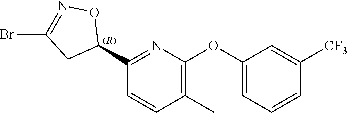

T-B-97

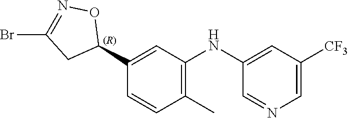

T-B-98

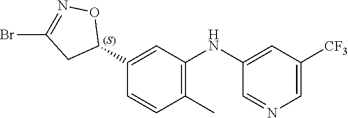

T-B-99

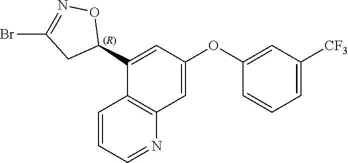

T-B-100

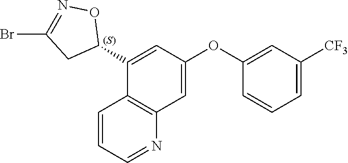

T-B-101

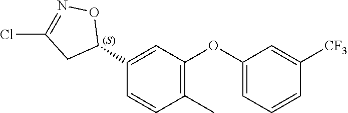

T-B-102

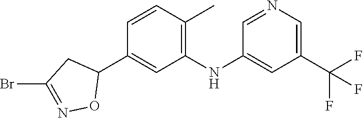 [SMILES: Cc1ccc(C2CC(Br)=NO2)cc1Nc1cncc(C(F)(F)F)c1]

T-B-103

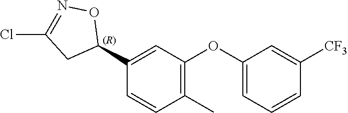

T-B-104

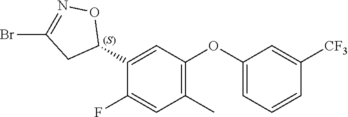

T-B-105

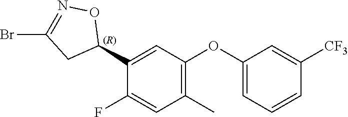

T-B-106

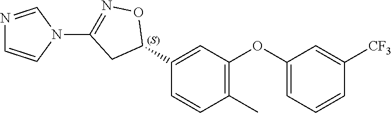

T-B-107

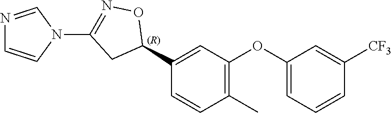

T-B-108

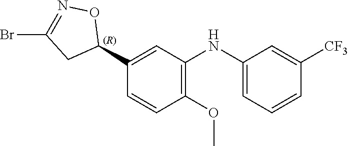

T-B-109

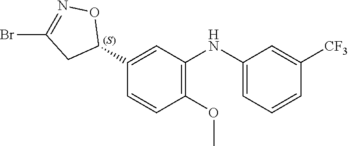

T-B-110

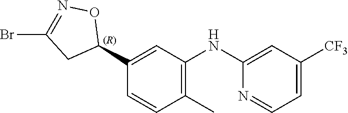

T-B-111

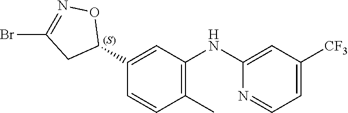

T-B-112

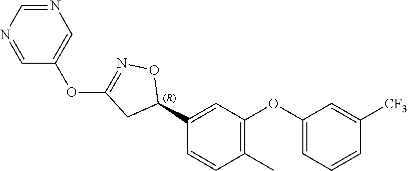

T-B-113

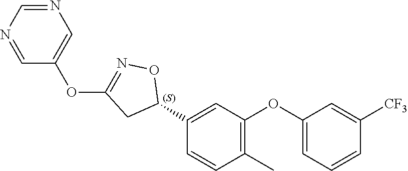

T-B-114

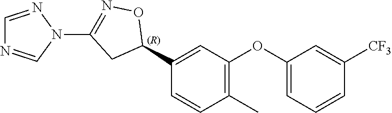

T-B-115

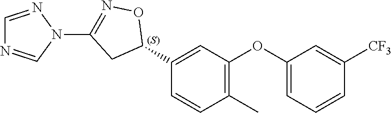

T-B-116

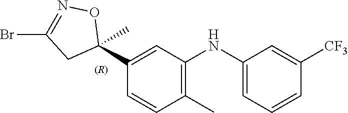

T-B-117

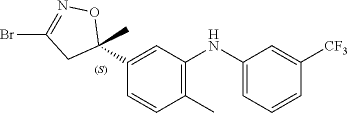

T-B-118

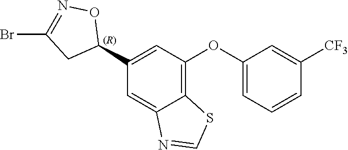

T-B-119

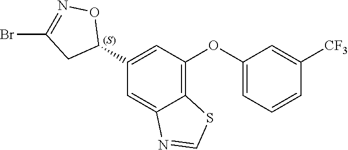

T-B-120

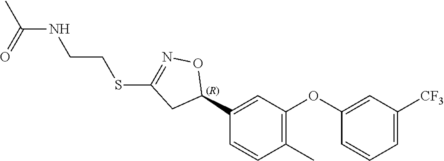

T-B-121

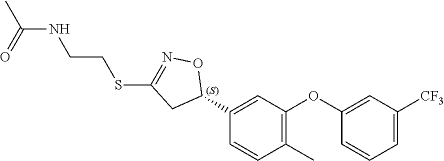

T-B-122

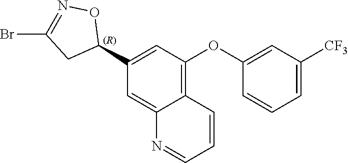

T-B-123

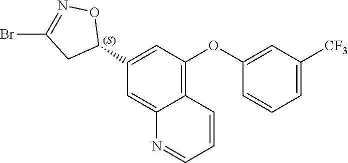

T-B-124

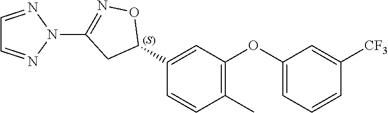

T-B-125

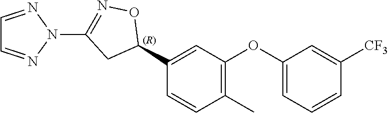

T-B-126

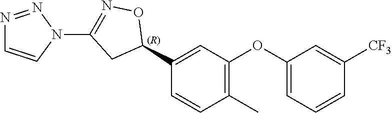

T-B-127

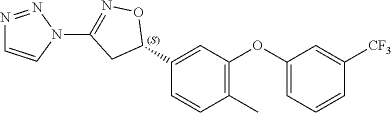

T-B-128

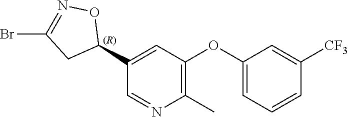

T-B-129

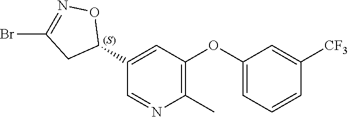

T-B-130

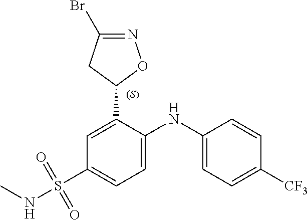

T-B-131

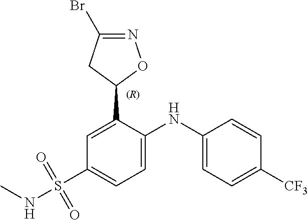

T-B-132

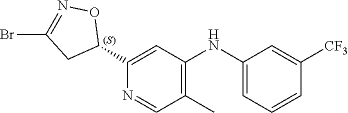

T-B-133

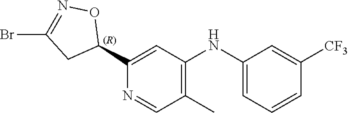

T-B-134

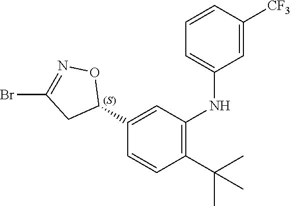

T-B-135

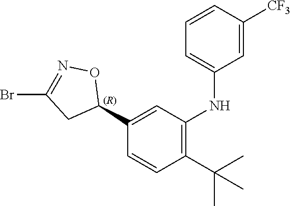

T-B-136

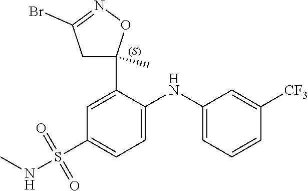

T-B-137

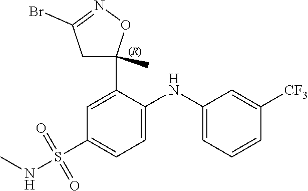

T-B-138

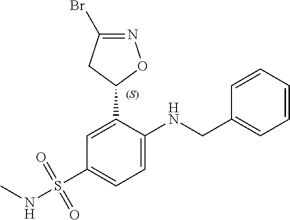

T-B-139

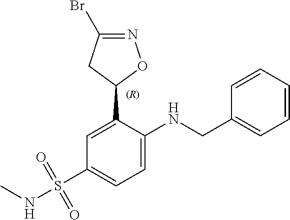

T-B-140

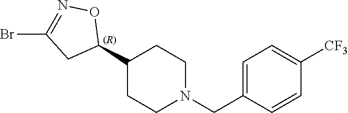

T-B-141

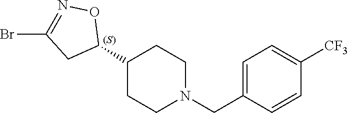

T-B-142

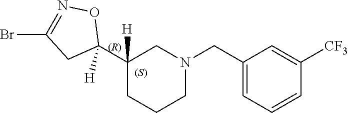

T-B-143

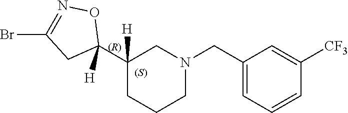

T-B-144

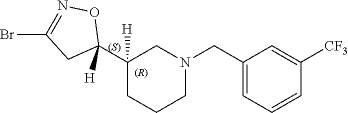

T-B-145

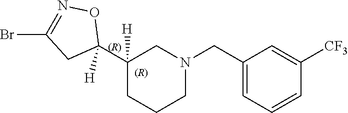

T-B-146

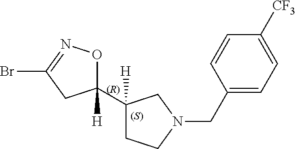

T-B-147

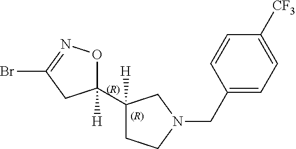

T-B-148

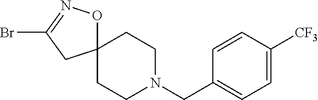 [SMILES: FC(F)(F)c1ccc(CN2CCC3(CC2)CC(Br)=NO3)cc1]

T-B-149

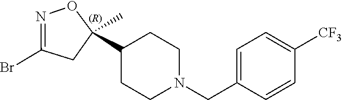

T-B-150

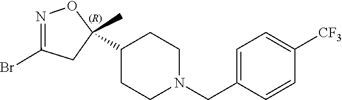

T-B-151

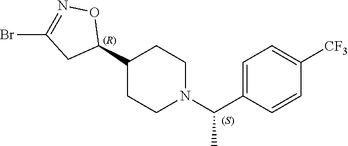

T-B-152

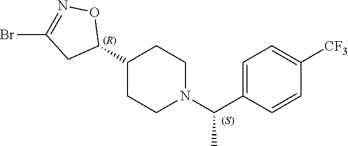

T-B-153

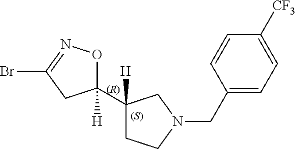

T-B-154

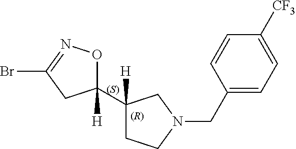

T-B-155

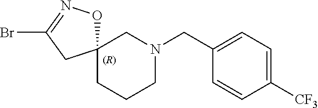

T-B-156

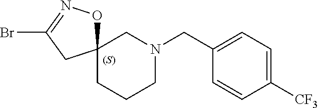

T-B-157

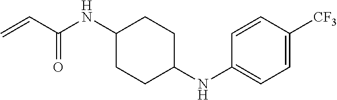 [SMILES: C=CC(=O)NC1CCC(Nc2ccc(C(F)(F)F)cc2)CC1]

T-B-158

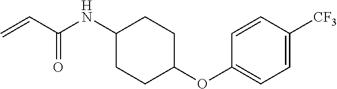 [SMILES: C=CC(=O)NC1CCC(Oc2ccc(C(F)(F)F)cc2)CC1]

T-B-159

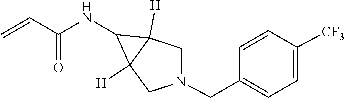 [SMILES: C=CC(=O)NC1C2CN(Cc3ccc(C(F)(F)F)cc3)CC21]

T-B-160

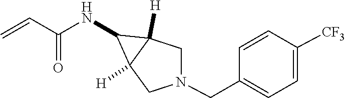

T-B-161

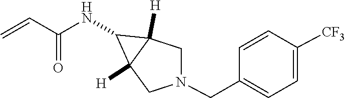

T-B-162

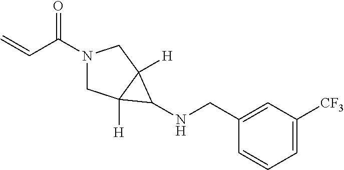 [SMILES: C=CC(=O)N1CC2C(C1)C2NCc1cccc(C(F)(F)F)c1]

T-B-163

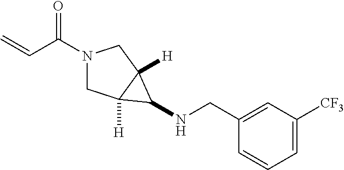

T-B-164

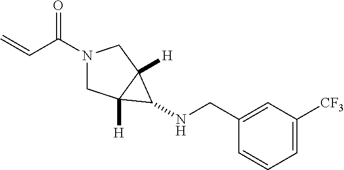

T-B-165

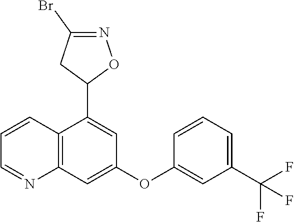 [SMILES: FC(F)(F)c1cccc(Oc2cc(C3CC(Br)=NO3)c3cccnc3c2)c1]

T-B-166

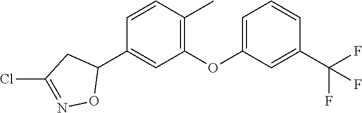 [SMILES: Cc1ccc(C2CC(Cl)=NO2)cc1Oc1cccc(C(F)(F)F)c1]

T-B-167

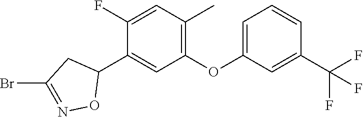 [SMILES: Cc1cc(F)c(C2CC(Br)=NO2)cc1Oc1cccc(C(F)(F)F)c1]

T-B-168

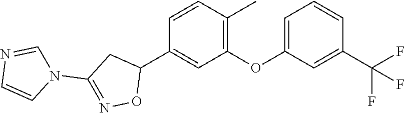 [SMILES: Cc1ccc(C2CC(n3ccnc3)=NO2)cc1Oc1cccc(C(F)(F)F)c1]

T-B-169

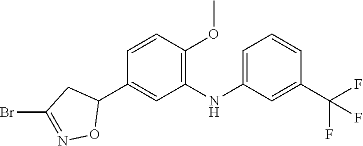 [SMILES: COc1ccc(C2CC(Br)=NO2)cc1Nc1cccc(C(F)(F)F)c1]

T-B-170

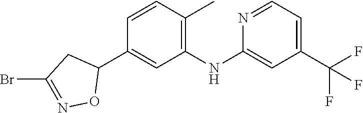 [SMILES: Cc1ccc(C2CC(Br)=NO2)cc1Nc1cc(C(F)(F)F)ccn1]

T-B-171

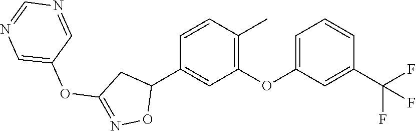 [SMILES: Cc1ccc(C2CC(Oc3cncnc3)=NO2)cc1Oc1cccc(C(F)(F)F)c1]

T-B-172

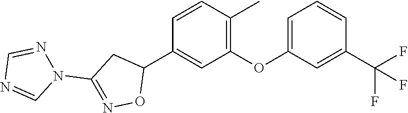 [SMILES: Cc1ccc(C2CC(n3cncn3)=NO2)cc1Oc1cccc(C(F)(F)F)c1]

T-B-173

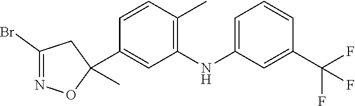 [SMILES: Cc1ccc(C2(C)CC(Br)=NO2)cc1Nc1cccc(C(F)(F)F)c1]

T-B-174

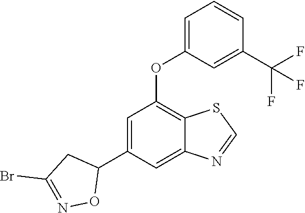 [SMILES: FC(F)(F)c1cccc(Oc2cc(C3CC(Br)=NO3)cc3ncsc23)c1]

T-B-175

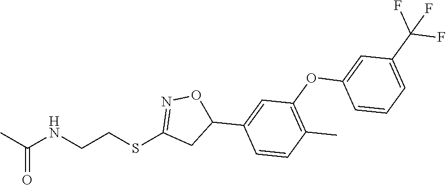 [SMILES: CC(=O)NCCSC1=NOC(c2ccc(C)c(Oc3cccc(C(F)(F)F)c3)c2)C1]

T-B-176

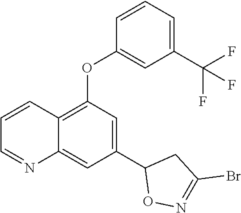 [SMILES: FC(F)(F)c1cccc(Oc2cc(C3CC(Br)=NO3)cc3ncccc23)c1]

T-B-177

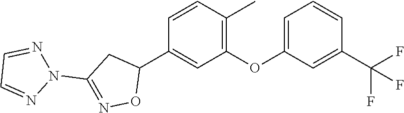 [SMILES: Cc1ccc(C2CC(n3nccn3)=NO2)cc1Oc1cccc(C(F)(F)F)c1]

T-B-178

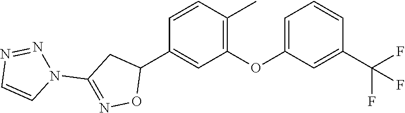 [SMILES: Cc1ccc(C2CC(n3ccnn3)=NO2)cc1Oc1cccc(C(F)(F)F)c1]

T-B-179

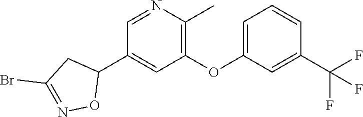 [SMILES: Cc1ncc(C2CC(Br)=NO2)cc1Oc1cccc(C(F)(F)F)c1]

T-B-180

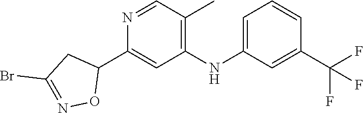 [SMILES: Cc1cnc(C2CC(Br)=NO2)cc1Nc1cccc(C(F)(F)F)c1]

T-B-181

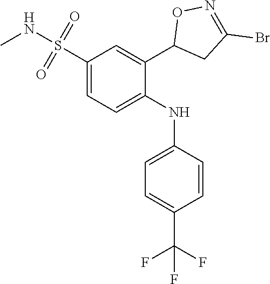 [SMILES: CNS(=O)(=O)c1ccc(Nc2ccc(C(F)(F)F)cc2)c(C2CC(Br)=NO2)c1]

T-B-182

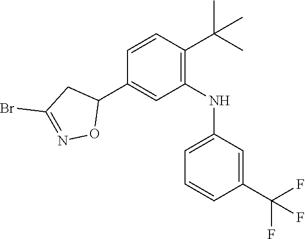 [SMILES: CC(C)(C)c1ccc(C2CC(Br)=NO2)cc1Nc1cccc(C(F)(F)F)c1]

T-B-183

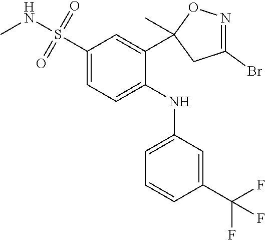 [SMILES: CNS(=O)(=O)c1ccc(Nc2cccc(C(F)(F)F)c2)c(C2(C)CC(Br)=NO2)c1]

T-B-184

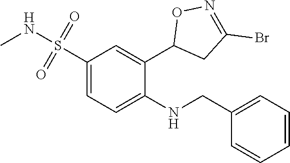 [SMILES: CNS(=O)(=O)c1ccc(NCc2ccccc2)c(C2CC(Br)=NO2)c1]

T-B-185

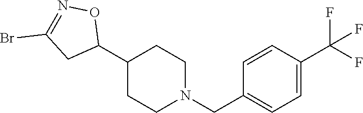 [SMILES: FC(F)(F)c1ccc(CN2CCC(C3CC(Br)=NO3)CC2)cc1]

T-B-186

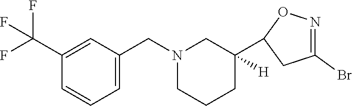 [SMILES: FC(F)(F)c1cccc(CN2CCC[C@H](C3CC(Br)=NO3)C2)c1]

T-B-187

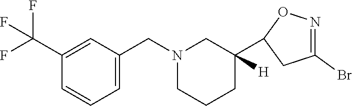 [SMILES: FC(F)(F)c1cccc(CN2CCC[C@@H](C3CC(Br)=NO3)C2)c1]

T-B-188

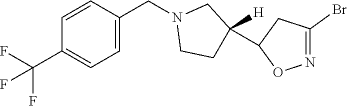 [SMILES: FC(F)(F)c1ccc(CN2CC[C@@H](C3CC(Br)=NO3)C2)cc1]

T-B-189

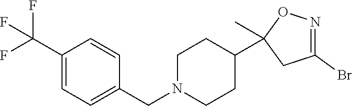 [SMILES: CC1(C2CCN(Cc3ccc(C(F)(F)F)cc3)CC2)CC(Br)=NO1]

T-B-190

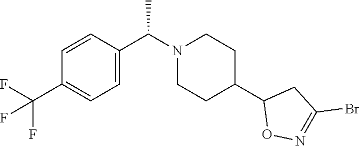 [SMILES: C[C@@H](c1ccc(C(F)(F)F)cc1)N1CCC(C2CC(Br)=NO2)CC1]

T-B-191

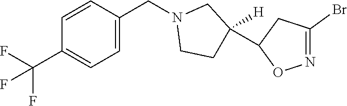 [SMILES: FC(F)(F)c1ccc(CN2CC[C@H](C3CC(Br)=NO3)C2)cc1]

T-B-192

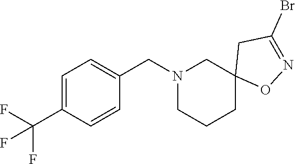 [SMILES: FC(F)(F)c1ccc(CN2CCCC3(CC(Br)=NO3)C2)cc1]

T-B-193

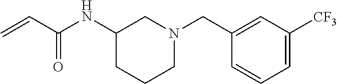 [SMILES: C=CC(=O)NC1CCCN(Cc2cccc(C(F)(F)F)c2)C1]

T-B-194

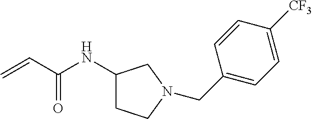 [SMILES: C=CC(=O)NC1CCN(Cc2ccc(C(F)(F)F)cc2)C1]

T-B-195

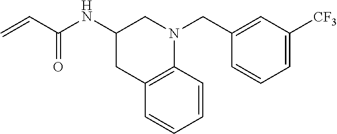 [SMILES: C=CC(=O)NC1Cc2ccccc2N(Cc2cccc(C(F)(F)F)c2)C1]

T-B-196

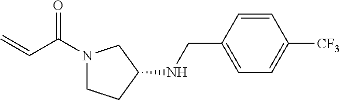 [SMILES: C=CC(=O)N1CC[C@@H](NCc2ccc(C(F)(F)F)cc2)C1]

T-B-197

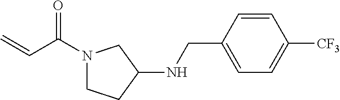 [SMILES: C=CC(=O)N1CCC(NCc2ccc(C(F)(F)F)cc2)C1]

T-B-198

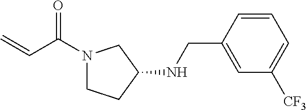 [SMILES: C=CC(=O)N1CC[C@@H](NCc2cccc(C(F)(F)F)c2)C1]

T-B-199

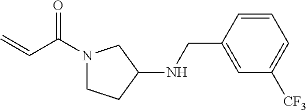 [SMILES: C=CC(=O)N1CCC(NCc2cccc(C(F)(F)F)c2)C1]

T-B-200

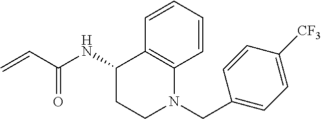 [SMILES: C=CC(=O)N[C@H]1CCN(Cc2ccc(C(F)(F)F)cc2)c2ccccc21]

T-B-201

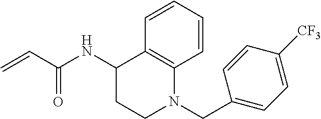 [SMILES: C=CC(=O)NC1CCN(Cc2ccc(C(F)(F)F)cc2)c2ccccc21]

T-B-202

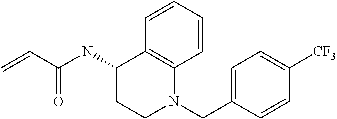 [SMILES: C=CC(=O)N[C@H]1CCN(Cc2ccc(C(F)(F)F)cc2)c2ccccc21]

T-B-203

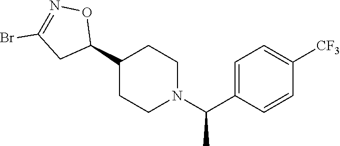 [SMILES: C[C@H](c1ccc(C(F)(F)F)cc1)N1CCC([C@H]2CC(Br)=NO2)CC1]

T-B-204

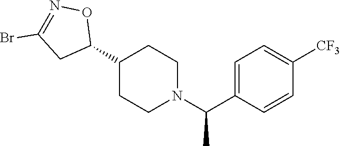 [SMILES: C[C@H](c1ccc(C(F)(F)F)cc1)N1CCC([C@@H]2CC(Br)=NO2)CC1]

T-B-205

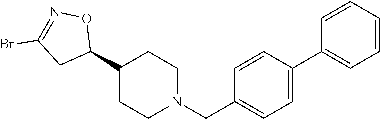 [SMILES: BrC1=NO[C@@H](C2CCN(Cc3ccc(-c4ccccc4)cc3)CC2)C1]

T-B-206

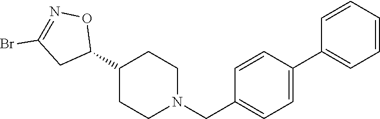 [SMILES: BrC1=NO[C@H](C2CCN(Cc3ccc(-c4ccccc4)cc3)CC2)C1]

T-B-207

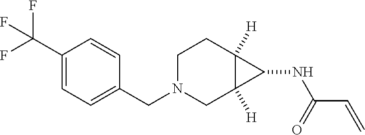 [SMILES: C=CC(=O)N[C@H]1[C@@H]2CCN(Cc3ccc(C(F)(F)F)cc3)C[C@@H]21]

T-B-208

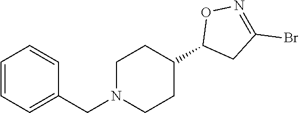 [SMILES: BrC1=NO[C@@H](C2CCN(Cc3ccccc3)CC2)C1]

T-B-209

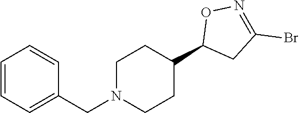 [SMILES: BrC1=NO[C@H](C2CCN(Cc3ccccc3)CC2)C1]

T-B-210

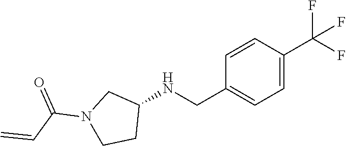 [SMILES: C=CC(=O)N1CC[C@@H](NCc2ccc(C(F)(F)F)cc2)C1]

T-B-211

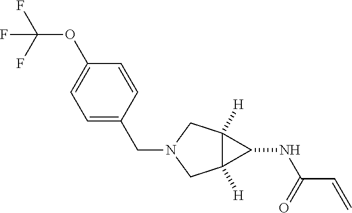 [SMILES: C=CC(=O)N[C@H]1[C@@H]2CN(Cc3ccc(OC(F)(F)F)cc3)C[C@@H]21]

T-B-212

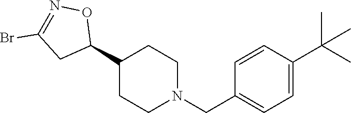 [SMILES: CC(C)(C)c1ccc(CN2CCC([C@H]3CC(Br)=NO3)CC2)cc1]

T-B-213

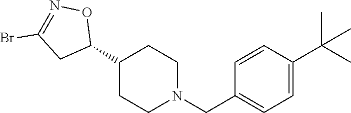 [SMILES: CC(C)(C)c1ccc(CN2CCC([C@@H]3CC(Br)=NO3)CC2)cc1]

T-B-214

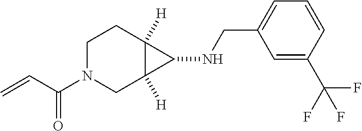 [SMILES: C=CC(=O)N1CC[C@@H]2[C@H](C1)[C@H]2NCc1cccc(C(F)(F)F)c1]

T-B-217

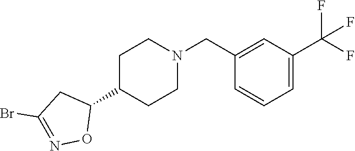 [SMILES: FC(F)(F)c1cccc(CN2CCC([C@H]3CC(Br)=NO3)CC2)c1]

T-B-218

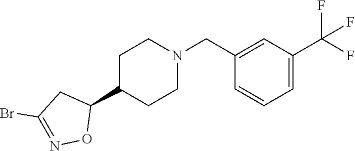 [SMILES: FC(F)(F)c1cccc(CN2CCC([C@@H]3CC(Br)=NO3)CC2)c1]

T-B-219

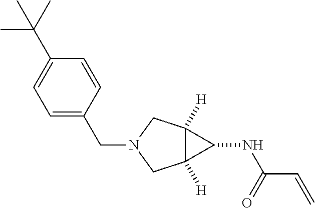 [SMILES: C=CC(=O)N[C@H]1[C@@H]2CN(Cc3ccc(C(C)(C)C)cc3)C[C@@H]21]

T-B-220

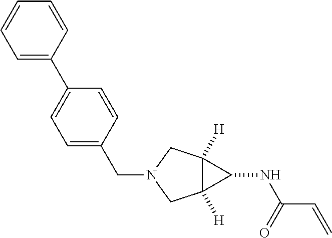 [SMILES: C=CC(=O)N[C@H]1[C@@H]2CN(Cc3ccc(-c4ccccc4)cc3)C[C@@H]21]

T-B-221

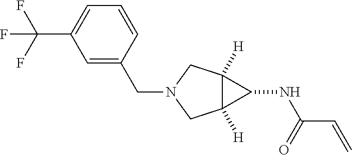 [SMILES: C=CC(=O)N[C@H]1[C@@H]2CN(Cc3cccc(C(F)(F)F)c3)C[C@@H]21]

T-B-222

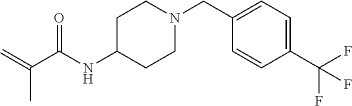 [SMILES: C=C(C)C(=O)NC1CCN(Cc2ccc(C(F)(F)F)cc2)CC1]

T-B-223

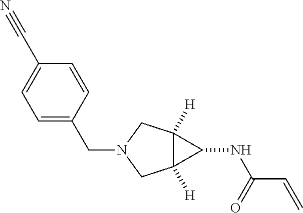 [SMILES: C=CC(=O)N[C@H]1[C@@H]2CN(Cc3ccc(C#N)cc3)C[C@@H]21]

T-B-224

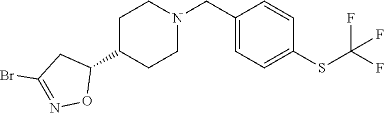 [SMILES: FC(F)(F)Sc1ccc(CN2CCC([C@H]3CC(Br)=NO3)CC2)cc1]

T-B-225

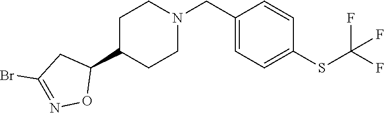 [SMILES: FC(F)(F)Sc1ccc(CN2CCC([C@@H]3CC(Br)=NO3)CC2)cc1]

T-B-226

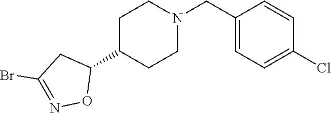 [SMILES: Clc1ccc(CN2CCC([C@H]3CC(Br)=NO3)CC2)cc1]

T-B-227

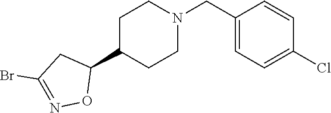 [SMILES: Clc1ccc(CN2CCC([C@@H]3CC(Br)=NO3)CC2)cc1]

T-B-228

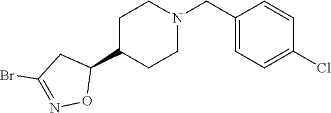 [SMILES: Clc1ccc(CN2CCC([C@@H]3CC(Br)=NO3)CC2)cc1]

T-B-229

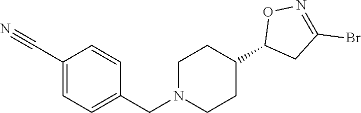 [SMILES: N#Cc1ccc(CN2CCC([C@H]3CC(Br)=NO3)CC2)cc1]

T-B-230

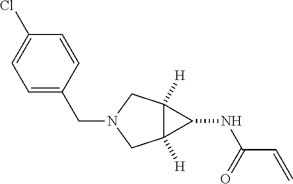 [SMILES: C=CC(=O)N[C@H]1[C@@H]2CN(Cc3ccc(Cl)cc3)C[C@@H]21]

T-B-231

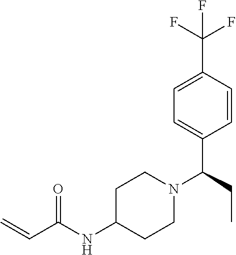 [SMILES: C=CC(=O)NC1CCN([C@H](CC)c2ccc(C(F)(F)F)cc2)CC1]

T-B-232

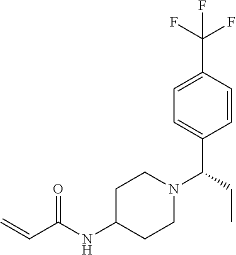 [SMILES: C=CC(=O)NC1CCN([C@@H](CC)c2ccc(C(F)(F)F)cc2)CC1]

T-B-233

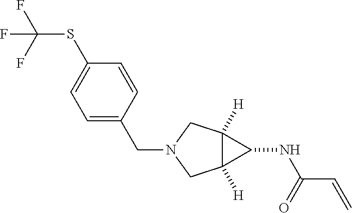 [SMILES: C=CC(=O)N[C@H]1[C@@H]2CN(Cc3ccc(SC(F)(F)F)cc3)C[C@@H]21]

T-B-234

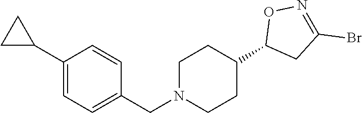 [SMILES: BrC1=NO[C@@H](C2CCN(Cc3ccc(C4CC4)cc3)CC2)C1]

T-B-235

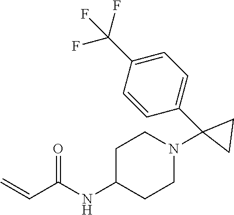 [SMILES: C=CC(=O)NC1CCN(C2(c3ccc(C(F)(F)F)cc3)CC2)CC1]

T-B-236

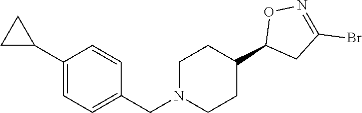 [SMILES: BrC1=NO[C@H](C2CCN(Cc3ccc(C4CC4)cc3)CC2)C1]

T-B-237

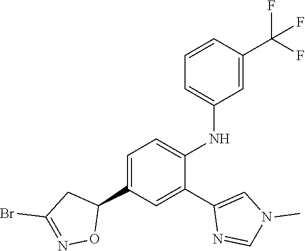 [SMILES: Cn1cnc(-c2cc([C@@H]3CC(Br)=NO3)ccc2Nc2cccc(C(F)(F)F)c2)c1]

T-B-238

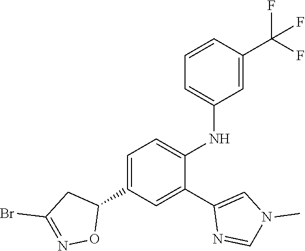 [SMILES: Cn1cnc(-c2cc([C@H]3CC(Br)=NO3)ccc2Nc2cccc(C(F)(F)F)c2)c1]

T-B-239

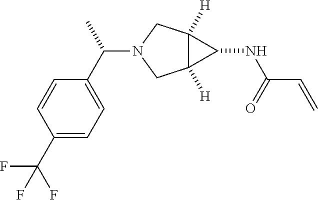 [SMILES: C=CC(=O)N[C@H]1[C@@H]2CN([C@@H](C)c3ccc(C(F)(F)F)cc3)C[C@@H]21]

T-B-240

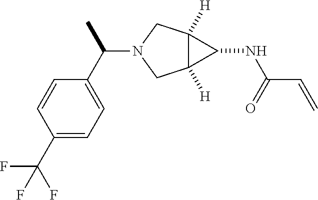 [SMILES: C=CC(=O)N[C@H]1[C@@H]2CN([C@H](C)c3ccc(C(F)(F)F)cc3)C[C@@H]21]

T-B-241

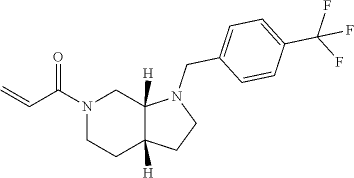 [SMILES: C=CC(=O)N1CC[C@H]2CCN(Cc3ccc(C(F)(F)F)cc3)[C@H]2C1]

T-B-242

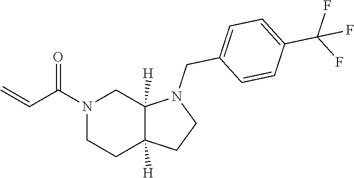 [SMILES: C=CC(=O)N1CC[C@@H]2CCN(Cc3ccc(C(F)(F)F)cc3)[C@@H]2C1]

T-B-243

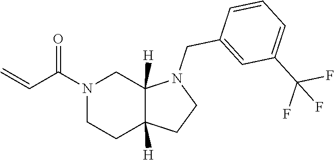 [SMILES: C=CC(=O)N1CC[C@H]2CCN(Cc3cccc(C(F)(F)F)c3)[C@H]2C1]

T-B-244

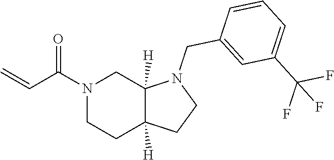 [SMILES: C=CC(=O)N1CC[C@@H]2CCN(Cc3cccc(C(F)(F)F)c3)[C@@H]2C1]

T-B-245

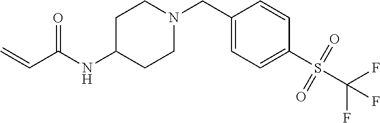 [SMILES: C=CC(=O)NC1CCN(Cc2ccc(S(=O)(=O)C(F)(F)F)cc2)CC1]

T-B-246

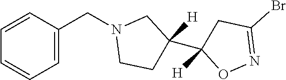 [SMILES: BrC1=NO[C@H]([C@@H]2CCN(Cc3ccccc3)C2)C1]

T-B-247

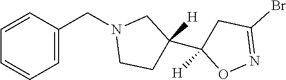 [SMILES: BrC1=NO[C@@H]([C@@H]2CCN(Cc3ccccc3)C2)C1]

T-B-248

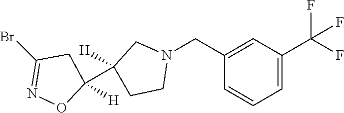 [SMILES: FC(F)(F)c1cccc(CN2CC[C@@H]([C@@H]3CC(Br)=NO3)C2)c1]

T-B-249

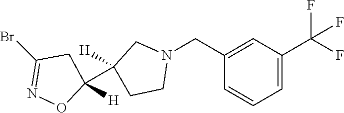 [SMILES: FC(F)(F)c1cccc(CN2CC[C@@H]([C@H]3CC(Br)=NO3)C2)c1]

T-B-250

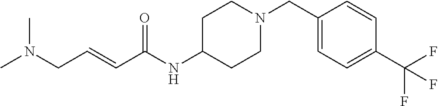 [SMILES: CN(C)C/C=C/C(=O)NC1CCN(Cc2ccc(C(F)(F)F)cc2)CC1]

T-B-251

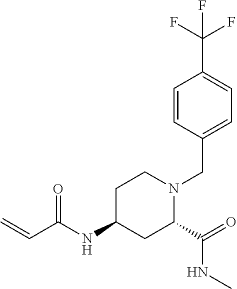 [SMILES: C=CC(=O)N[C@H]1CCN(Cc2ccc(C(F)(F)F)cc2)[C@H](C(=O)NC)C1]

T-B-252

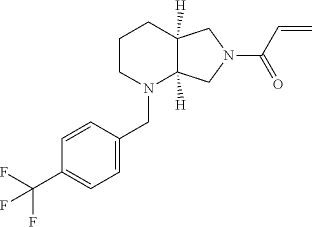 [SMILES: C=CC(=O)N1C[C@@H]2CCCN(Cc3ccc(C(F)(F)F)cc3)[C@@H]2C1]

T-B-253

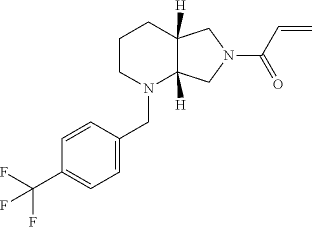 [SMILES: C=CC(=O)N1C[C@H]2CCCN(Cc3ccc(C(F)(F)F)cc3)[C@H]2C1]

T-B-254

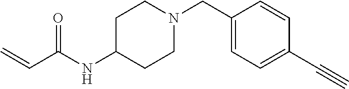 [SMILES: C#Cc1ccc(CN2CCC(NC(=O)C=C)CC2)cc1]

T-B-255

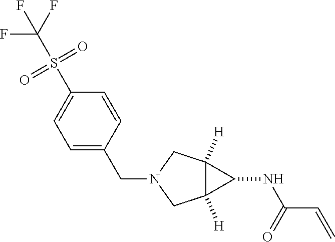 [SMILES: C=CC(=O)N[C@H]1[C@@H]2CN(Cc3ccc(S(=O)(=O)C(F)(F)F)cc3)C[C@@H]21]

T-B-256

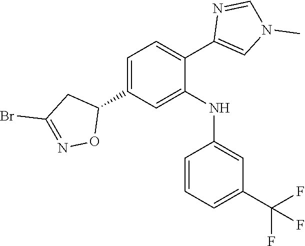 [SMILES: Cn1cnc(-c2ccc([C@H]3CC(Br)=NO3)cc2Nc2cccc(C(F)(F)F)c2)c1]

T-B-257

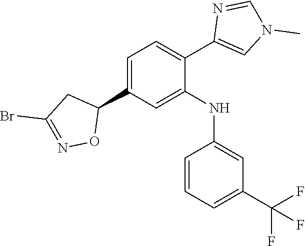 [SMILES: Cn1cnc(-c2ccc([C@@H]3CC(Br)=NO3)cc2Nc2cccc(C(F)(F)F)c2)c1]

T-B-258

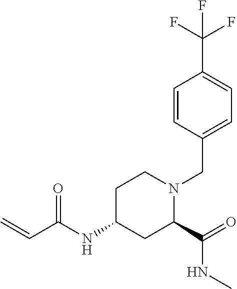 [SMILES: C=CC(=O)N[C@@H]1CCN(Cc2ccc(C(F)(F)F)cc2)[C@@H](C(=O)NC)C1]

T-B-259

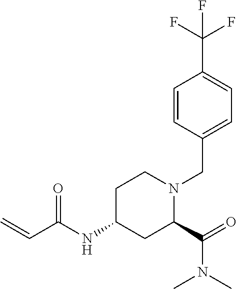 [SMILES: C=CC(=O)N[C@@H]1CCN(Cc2ccc(C(F)(F)F)cc2)[C@@H](C(=O)N(C)C)C1]

T-B-260

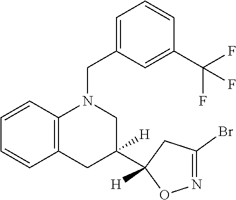 [SMILES: FC(F)(F)c1cccc(CN2C[C@@H]([C@@H]3CC(Br)=NO3)Cc3ccccc32)c1]

T-B-261

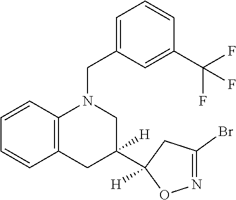 [SMILES: FC(F)(F)c1cccc(CN2C[C@@H]([C@H]3CC(Br)=NO3)Cc3ccccc32)c1]

T-B-262

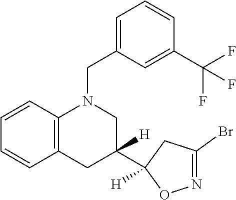 [SMILES: FC(F)(F)c1cccc(CN2C[C@H]([C@H]3CC(Br)=NO3)Cc3ccccc32)c1]

T-B-263

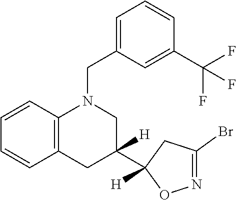 [SMILES: FC(F)(F)c1cccc(CN2C[C@H]([C@@H]3CC(Br)=NO3)Cc3ccccc32)c1]

T-B-264

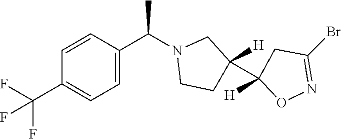 [SMILES: C[C@H](c1ccc(C(F)(F)F)cc1)N1CC[C@@H]([C@@H]2CC(Br)=NO2)C1]

T-B-265

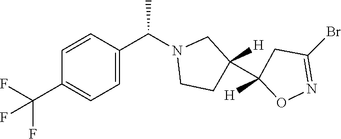 [SMILES: C[C@@H](c1ccc(C(F)(F)F)cc1)N1CC[C@@H]([C@@H]2CC(Br)=NO2)C1]

T-B-266

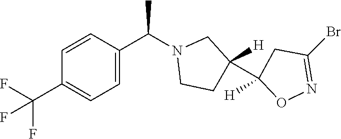 [SMILES: C[C@H](c1ccc(C(F)(F)F)cc1)N1CC[C@@H]([C@H]2CC(Br)=NO2)C1]

T-B-267

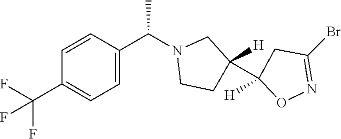 [SMILES: C[C@@H](c1ccc(C(F)(F)F)cc1)N1CC[C@@H]([C@H]2CC(Br)=NO2)C1]

T-B-268

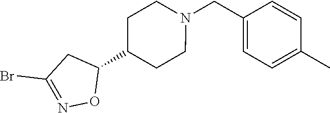 [SMILES: Cc1ccc(CN2CCC([C@H]3CC(Br)=NO3)CC2)cc1]

T-B-269

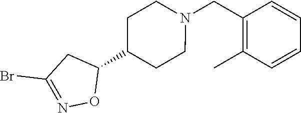 [SMILES: Cc1ccccc1CN1CCC([C@H]2CC(Br)=NO2)CC1]

T-B-270

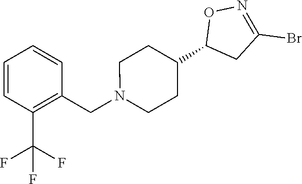 [SMILES: FC(F)(F)c1ccccc1CN1CCC([C@H]2CC(Br)=NO2)CC1]

T-B-271

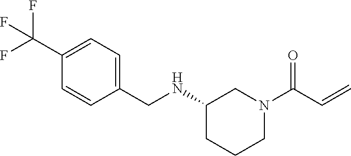 [SMILES: C=CC(=O)N1CCC[C@H](NCc2ccc(C(F)(F)F)cc2)C1]

T-B-272

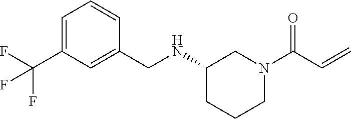 [SMILES: C=CC(=O)N1CCC[C@H](NCc2cccc(C(F)(F)F)c2)C1]

T-B-273

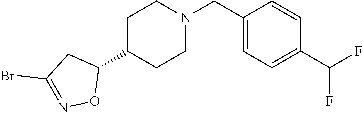 [SMILES: FC(F)c1ccc(CN2CCC([C@H]3CC(Br)=NO3)CC2)cc1]

T-B-274

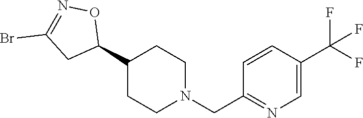 [SMILES: FC(F)(F)c1ccc(CN2CCC([C@H]3CC(Br)=NO3)CC2)nc1]

T-B-275

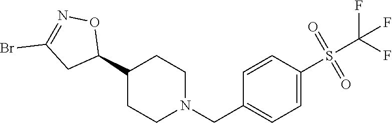 [SMILES: O=S(=O)(c1ccc(CN2CCC([C@H]3CC(Br)=NO3)CC2)cc1)C(F)(F)F]

T-B-276

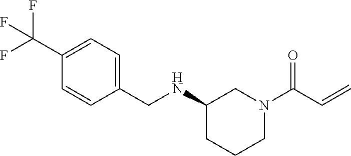 [SMILES: C=CC(=O)N1CCC[C@@H](NCc2ccc(C(F)(F)F)cc2)C1]

T-B-277

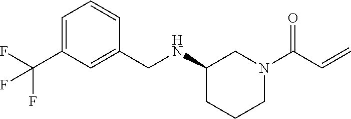 [SMILES: C=CC(=O)N1CCC[C@@H](NCc2cccc(C(F)(F)F)c2)C1]

T-B-278

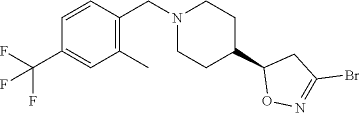 [SMILES: Cc1cc(C(F)(F)F)ccc1CN1CCC([C@H]2CC(Br)=NO2)CC1]

T-B-279

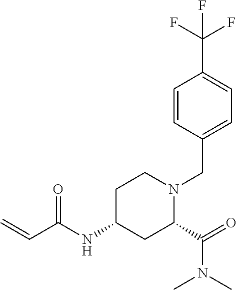 [SMILES: C=CC(=O)N[C@@H]1CCN(Cc2ccc(C(F)(F)F)cc2)[C@H](C(=O)N(C)C)C1]

T-B-280

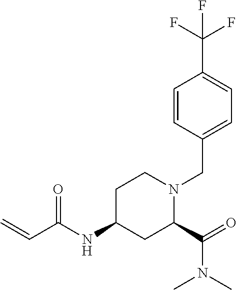 [SMILES: C=CC(=O)N[C@H]1CCN(Cc2ccc(C(F)(F)F)cc2)[C@@H](C(=O)N(C)C)C1]

T-B-281

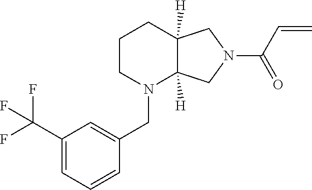 [SMILES: C=CC(=O)N1C[C@@H]2CCCN(Cc3cccc(C(F)(F)F)c3)[C@@H]2C1]

T-B-282

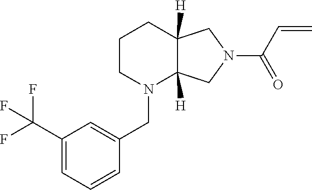 [SMILES: C=CC(=O)N1C[C@H]2CCCN(Cc3cccc(C(F)(F)F)c3)[C@H]2C1]

T-B-283

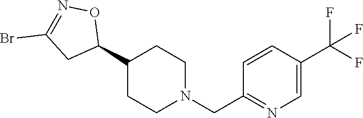 [SMILES: FC(F)(F)c1ccc(CN2CCC([C@H]3CC(Br)=NO3)CC2)nc1]

T-B-284

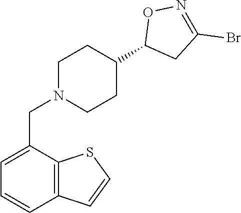 [SMILES: BrC1=NO[C@@H](C2CCN(Cc3cccc4ccsc34)CC2)C1]

T-B-285

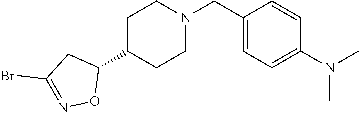 [SMILES: CN(C)c1ccc(CN2CCC([C@H]3CC(Br)=NO3)CC2)cc1]

T-B-286

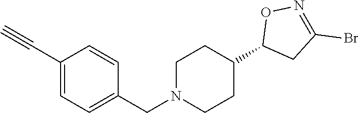 [SMILES: C#Cc1ccc(CN2CCC([C@H]3CC(Br)=NO3)CC2)cc1]

T-B-287

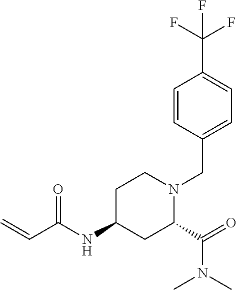 [SMILES: C=CC(=O)N[C@H]1CCN(Cc2ccc(C(F)(F)F)cc2)[C@H](C(=O)N(C)C)C1]

T-B-288

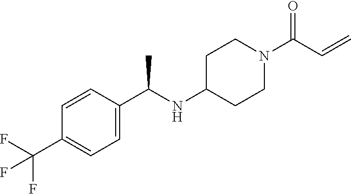 [SMILES: C=CC(=O)N1CCC(N[C@H](C)c2ccc(C(F)(F)F)cc2)CC1]

T-B-289

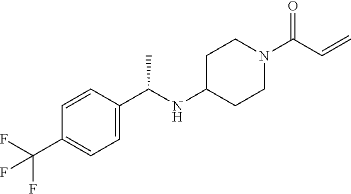 [SMILES: C=CC(=O)N1CCC(N[C@@H](C)c2ccc(C(F)(F)F)cc2)CC1]

T-B-290

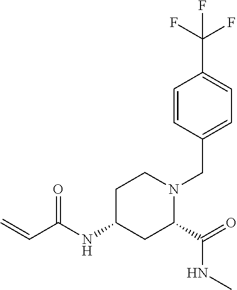 [SMILES: C=CC(=O)N[C@@H]1CCN(Cc2ccc(C(F)(F)F)cc2)[C@H](C(=O)NC)C1]

T-B-291

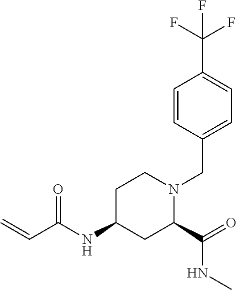 [SMILES: C=CC(=O)N[C@H]1CCN(Cc2ccc(C(F)(F)F)cc2)[C@@H](C(=O)NC)C1]

T-B-292

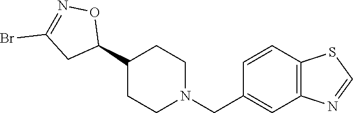 [SMILES: BrC1=NO[C@@H](C2CCN(Cc3ccc4scnc4c3)CC2)C1]

T-B-293

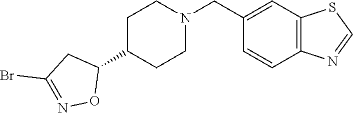 [SMILES: BrC1=NO[C@@H](C2CCN(Cc3ccc4ncsc4c3)CC2)C1]

T-B-294

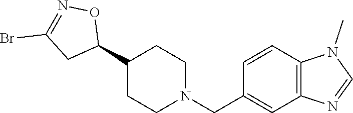 [SMILES: Cn1cnc2cc(CN3CCC([C@H]4CC(Br)=NO4)CC3)ccc21]

T-B-295

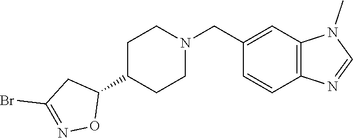 [SMILES: Cn1cnc2ccc(CN3CCC([C@H]4CC(Br)=NO4)CC3)cc21]

T-B-296

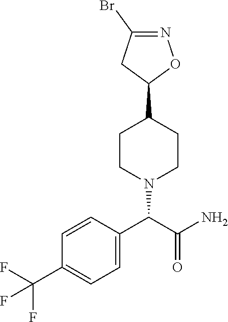 [SMILES: NC(=O)[C@H](c1ccc(C(F)(F)F)cc1)N1CCC([C@H]2CC(Br)=NO2)CC1]

T-B-297

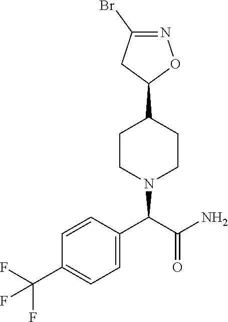 [SMILES: NC(=O)[C@@H](c1ccc(C(F)(F)F)cc1)N1CCC([C@H]2CC(Br)=NO2)CC1]

T-B-298

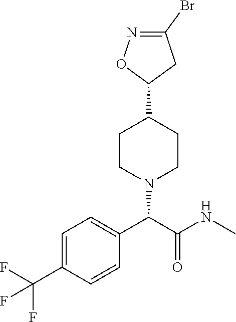 [SMILES: CNC(=O)[C@H](c1ccc(C(F)(F)F)cc1)N1CCC([C@H]2CC(Br)=NO2)CC1]

T-B-299

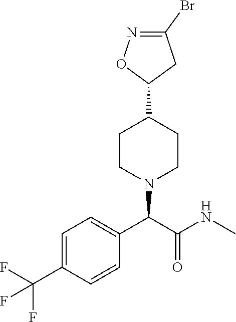 [SMILES: CNC(=O)[C@@H](c1ccc(C(F)(F)F)cc1)N1CCC([C@H]2CC(Br)=NO2)CC1]

T-B-300

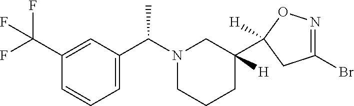 [SMILES: C[C@@H](c1cccc(C(F)(F)F)c1)N1CCC[C@@H]([C@@H]2CC(Br)=NO2)C1]

T-B-301

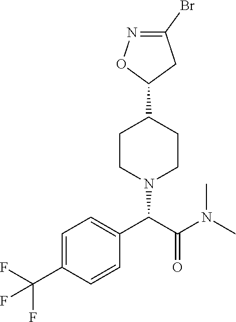 [SMILES: CN(C)C(=O)[C@H](c1ccc(C(F)(F)F)cc1)N1CCC([C@H]2CC(Br)=NO2)CC1]

T-B-302

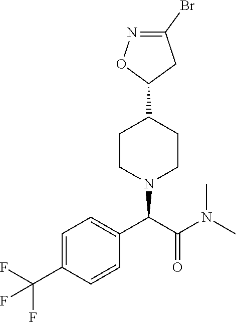 [SMILES: CN(C)C(=O)[C@@H](c1ccc(C(F)(F)F)cc1)N1CCC([C@H]2CC(Br)=NO2)CC1]

T-B-303

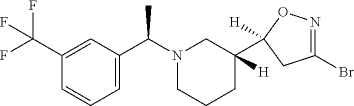 [SMILES: C[C@H](c1cccc(C(F)(F)F)c1)N1CCC[C@@H]([C@@H]2CC(Br)=NO2)C1]

T-B-304

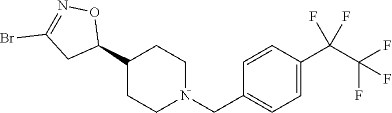 [SMILES: FC(F)(F)C(F)(F)c1ccc(CN2CCC([C@H]3CC(Br)=NO3)CC2)cc1]

T-B-305

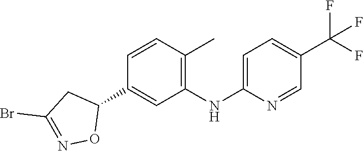 [SMILES: Cc1ccc([C@H]2CC(Br)=NO2)cc1Nc1ccc(C(F)(F)F)cn1]

T-B-306

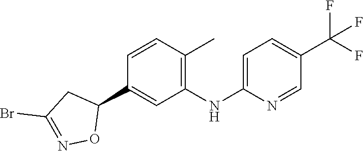 [SMILES: Cc1ccc([C@@H]2CC(Br)=NO2)cc1Nc1ccc(C(F)(F)F)cn1]

T-B-307

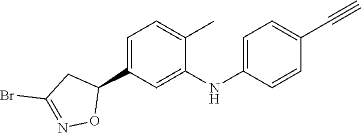 [SMILES: C#Cc1ccc(Nc2cc([C@@H]3CC(Br)=NO3)ccc2C)cc1]

T-B-308

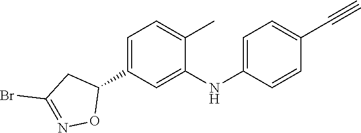 [SMILES: C#Cc1ccc(Nc2cc([C@H]3CC(Br)=NO3)ccc2C)cc1]

T-B-309

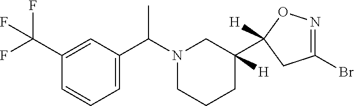 [SMILES: CC(c1cccc(C(F)(F)F)c1)N1CCC[C@@H]([C@H]2CC(Br)=NO2)C1]

T-B-310

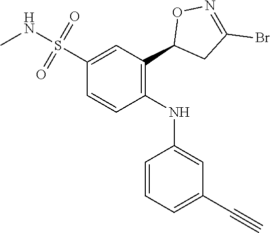 [SMILES: C#Cc1cccc(Nc2ccc(S(=O)(=O)NC)cc2[C@@H]2CC(Br)=NO2)c1]

T-B-311

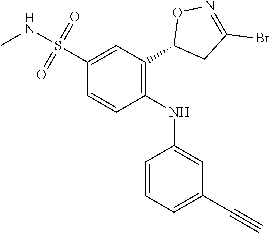 [SMILES: C#Cc1cccc(Nc2ccc(S(=O)(=O)NC)cc2[C@H]2CC(Br)=NO2)c1]

T-B-312

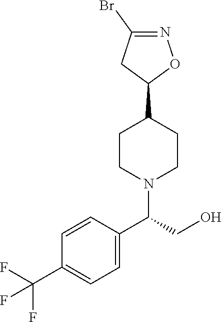 [SMILES: OC[C@H](c1ccc(C(F)(F)F)cc1)N1CCC([C@H]2CC(Br)=NO2)CC1]

T-B-313

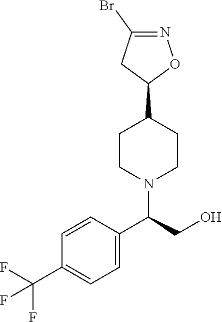 [SMILES: OC[C@@H](c1ccc(C(F)(F)F)cc1)N1CCC([C@H]2CC(Br)=NO2)CC1]

T-B-314

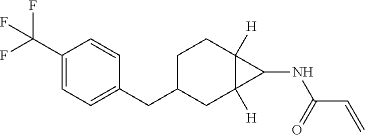 [SMILES: C=CC(=O)NC1C2CCC(Cc3ccc(C(F)(F)F)cc3)CC21]

T-B-315

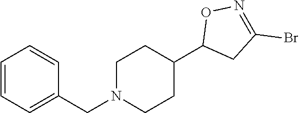 [SMILES: BrC1=NOC(C2CCN(Cc3ccccc3)CC2)C1]

T-B-316

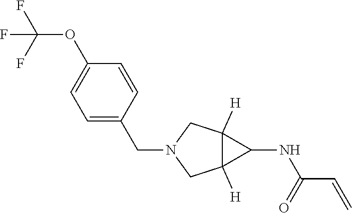 [SMILES: C=CC(=O)NC1C2CN(Cc3ccc(OC(F)(F)F)cc3)CC21]

T-B-317

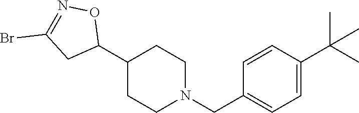 [SMILES: CC(C)(C)c1ccc(CN2CCC(C3CC(Br)=NO3)CC2)cc1]

T-B-318

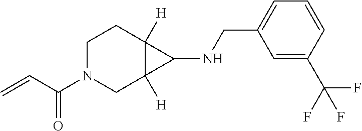 [SMILES: C=CC(=O)N1CCC2C(C1)C2NCc1cccc(C(F)(F)F)c1]

T-B-320

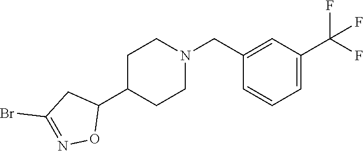 [SMILES: FC(F)(F)c1cccc(CN2CCC(C3CC(Br)=NO3)CC2)c1]

T-B-321

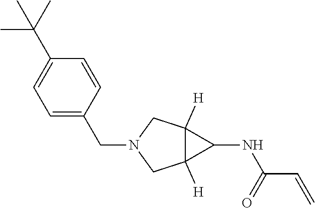 [SMILES: C=CC(=O)NC1C2CN(Cc3ccc(C(C)(C)C)cc3)CC21]

T-B-322

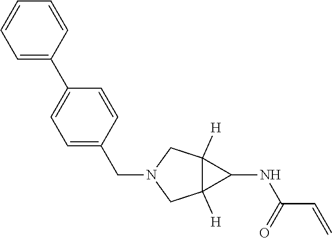 [SMILES: C=CC(=O)NC1C2CN(Cc3ccc(-c4ccccc4)cc3)CC21]

T-B-323

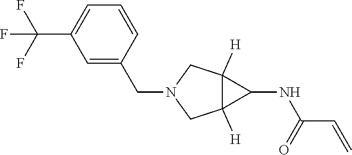 [SMILES: C=CC(=O)NC1C2CN(Cc3cccc(C(F)(F)F)c3)CC21]

T-B-324

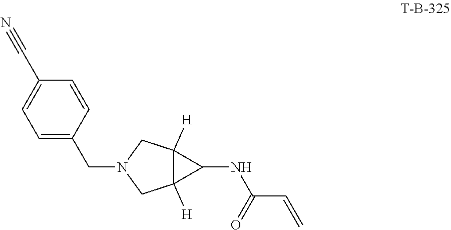 [SMILES: C=CC(=O)NC1C2CN(Cc3ccc(C#N)cc3)CC21]

T-B-325

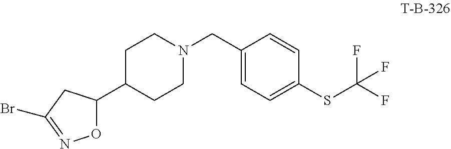 [SMILES: FC(F)(F)Sc1ccc(CN2CCC(C3CC(Br)=NO3)CC2)cc1]

T-B-326

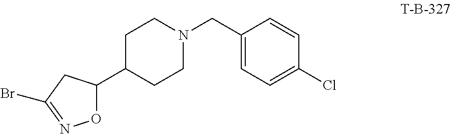 [SMILES: Clc1ccc(CN2CCC(C3CC(Br)=NO3)CC2)cc1]

T-B-327

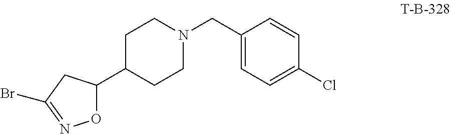 [SMILES: Clc1ccc(CN2CCC(C3CC(Br)=NO3)CC2)cc1]

T-B-328

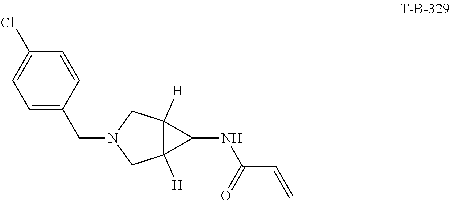 [SMILES: C=CC(=O)NC1C2CN(Cc3ccc(Cl)cc3)CC21]

T-B-329

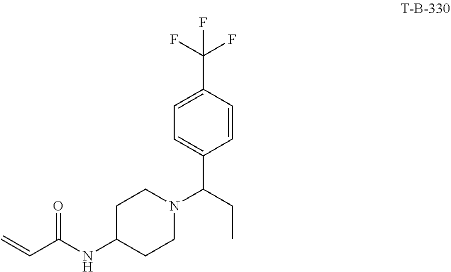 [SMILES: C=CC(=O)NC1CCN(C(CC)c2ccc(C(F)(F)F)cc2)CC1]

T-B-330

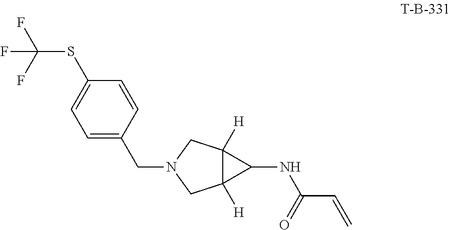 [SMILES: C=CC(=O)NC1C2CN(Cc3ccc(SC(F)(F)F)cc3)CC21]

T-B-331

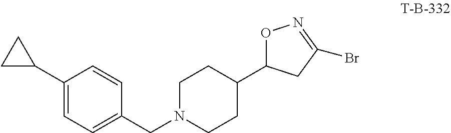 [SMILES: BrC1=NOC(C2CCN(Cc3ccc(C4CC4)cc3)CC2)C1]

T-B-332

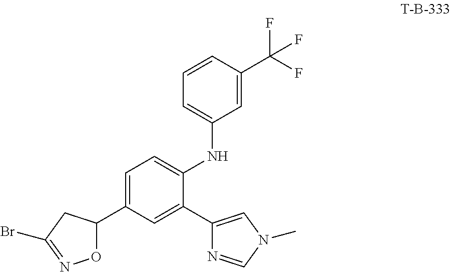 [SMILES: Cn1cnc(-c2cc(C3CC(Br)=NO3)ccc2Nc2cccc(C(F)(F)F)c2)c1]

T-B-333

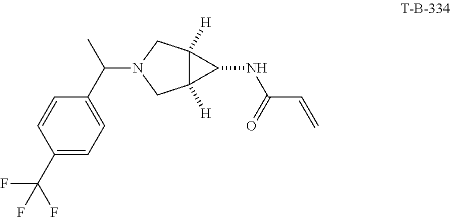 [SMILES: C=CC(=O)N[C@H]1[C@@H]2CN(C(C)c3ccc(C(F)(F)F)cc3)C[C@@H]21]

T-B-334

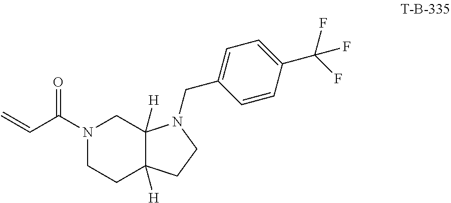 [SMILES: C=CC(=O)N1CCC2CCN(Cc3ccc(C(F)(F)F)cc3)C2C1]

T-B-335

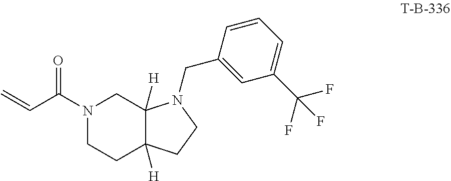 [SMILES: C=CC(=O)N1CCC2CCN(Cc3cccc(C(F)(F)F)c3)C2C1]

T-B-336

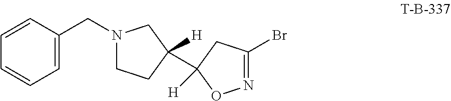 [SMILES: BrC1=NOC([C@@H]2CCN(Cc3ccccc3)C2)C1]

T-B-337

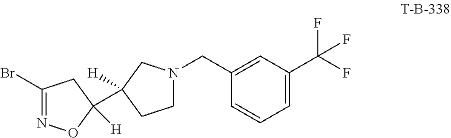 [SMILES: FC(F)(F)c1cccc(CN2CC[C@@H](C3CC(Br)=NO3)C2)c1]

T-B-338

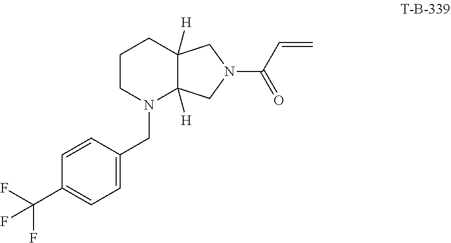 [SMILES: C=CC(=O)N1CC2CCCN(Cc3ccc(C(F)(F)F)cc3)C2C1]

T-B-339

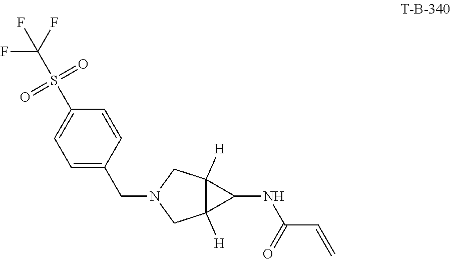 [SMILES: C=CC(=O)NC1C2CN(Cc3ccc(S(=O)(=O)C(F)(F)F)cc3)CC21]

T-B-340

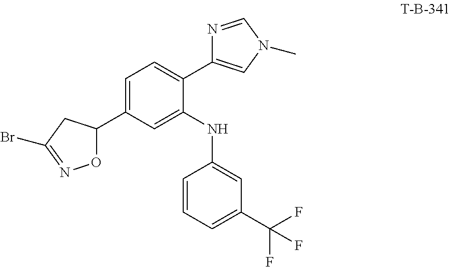 [SMILES: Cn1cnc(-c2ccc(C3CC(Br)=NO3)cc2Nc2cccc(C(F)(F)F)c2)c1]

T-B-341

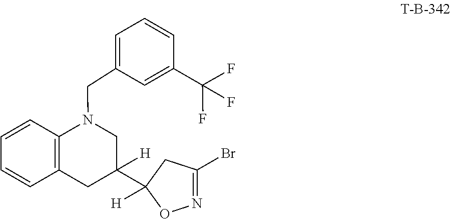 [SMILES: FC(F)(F)c1cccc(CN2CC(C3CC(Br)=NO3)Cc3ccccc32)c1]

T-B-342

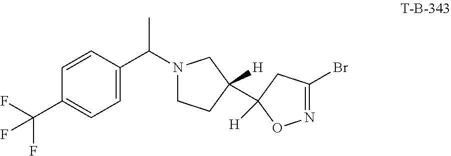 [SMILES: CC(c1ccc(C(F)(F)F)cc1)N1CC[C@@H](C2CC(Br)=NO2)C1]

T-B-343

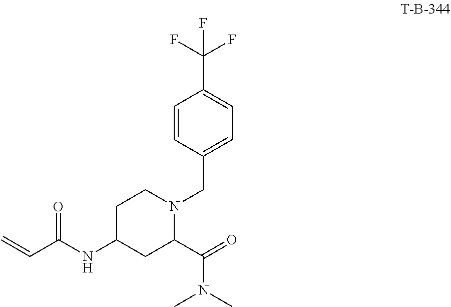 [SMILES: C=CC(=O)NC1CCN(Cc2ccc(C(F)(F)F)cc2)C(C(=O)N(C)C)C1]

T-B-344

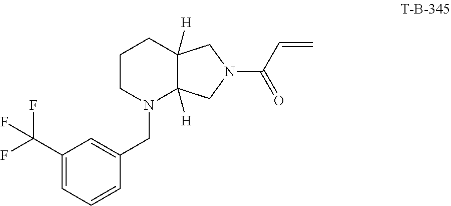 [SMILES: C=CC(=O)N1CC2CCCN(Cc3cccc(C(F)(F)F)c3)C2C1]

T-B-345

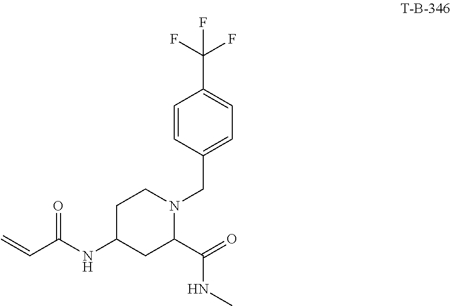 [SMILES: C=CC(=O)NC1CCN(Cc2ccc(C(F)(F)F)cc2)C(C(=O)NC)C1]

T-B-346

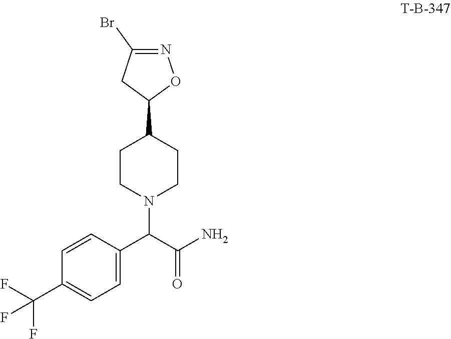

T-B-347

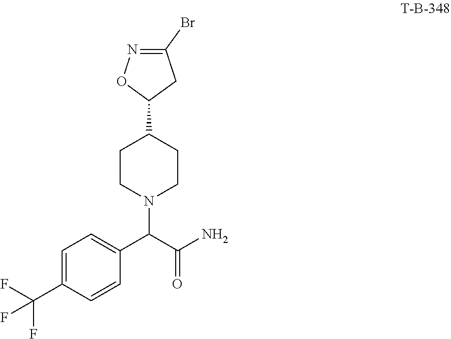

T-B-348

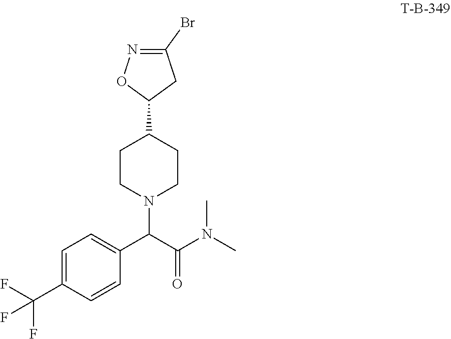

T-B-349

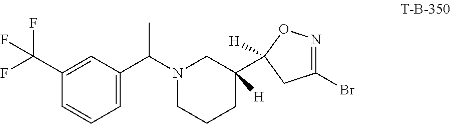 [SMILES: CC(c1cccc(C(F)(F)F)c1)N1CCC[C@@H]([C@@H]2CC(Br)=NO2)C1]

T-B-350

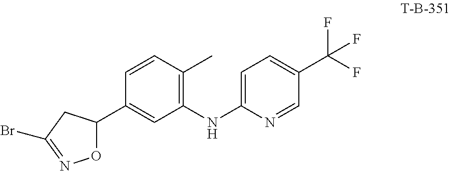 [SMILES: Cc1ccc(C2CC(Br)=NO2)cc1Nc1ccc(C(F)(F)F)cn1]

T-B-351

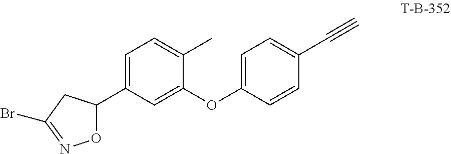 [SMILES: C#Cc1ccc(Oc2cc(C3CC(Br)=NO3)ccc2C)cc1]

T-B-352

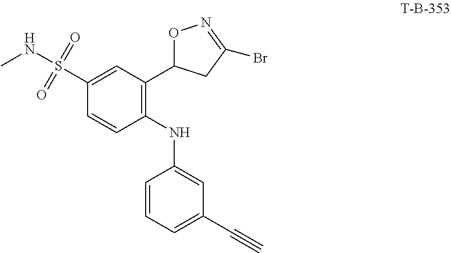 [SMILES: C#Cc1cccc(Nc2ccc(S(=O)(=O)NC)cc2C2CC(Br)=NO2)c1]

T-B-353

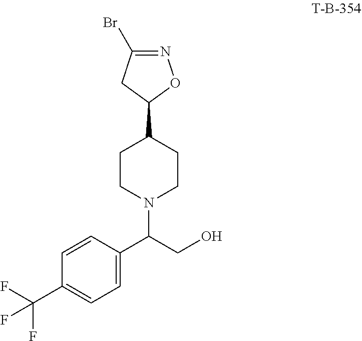 [SMILES: OCC(c1ccc(C(F)(F)F)cc1)N1CCC([C@H]2CC(Br)=NO2)CC1]

T-B-354

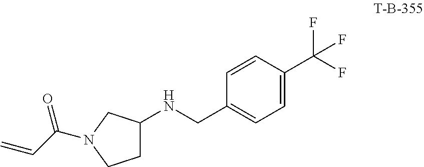 [SMILES: C=CC(=O)N1CCC(NCc2ccc(C(F)(F)F)cc2)C1]

T-B-355

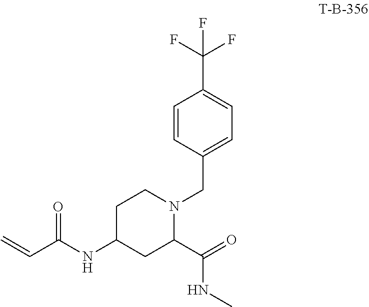 [SMILES: C=CC(=O)NC1CCN(Cc2ccc(C(F)(F)F)cc2)C(C(=O)NC)C1]

T-B-356

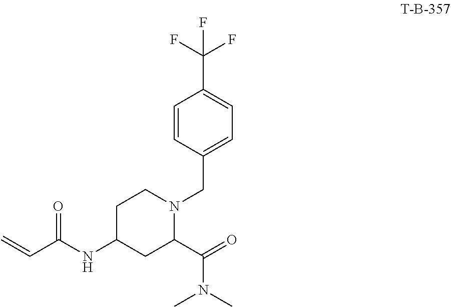 [SMILES: C=CC(=O)NC1CCN(Cc2ccc(C(F)(F)F)cc2)C(C(=O)N(C)C)C1]

T-B-357

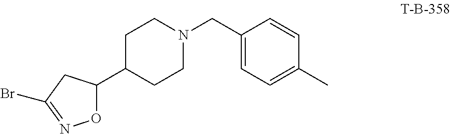 [SMILES: Cc1ccc(CN2CCC(C3CC(Br)=NO3)CC2)cc1]

T-B-358

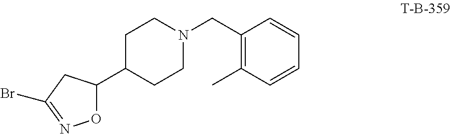 [SMILES: Cc1ccccc1CN1CCC(C2CC(Br)=NO2)CC1]

T-B-359

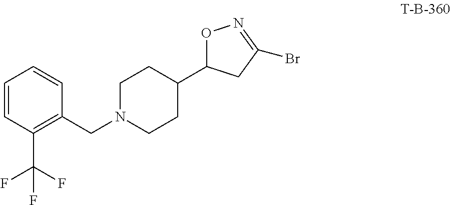 [SMILES: FC(F)(F)c1ccccc1CN1CCC(C2CC(Br)=NO2)CC1]

T-B-360

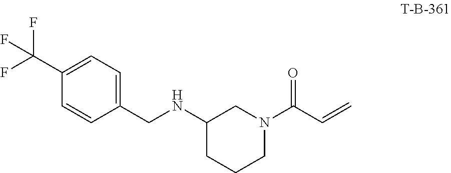 [SMILES: C=CC(=O)N1CCCC(NCc2ccc(C(F)(F)F)cc2)C1]

T-B-361

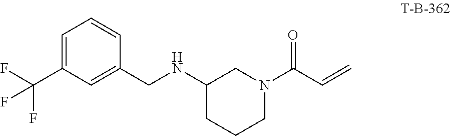 [SMILES: C=CC(=O)N1CCCC(NCc2cccc(C(F)(F)F)c2)C1]

T-B-362

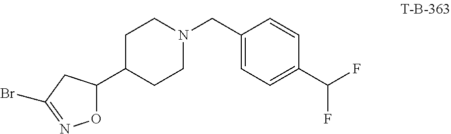 [SMILES: FC(F)c1ccc(CN2CCC(C3CC(Br)=NO3)CC2)cc1]

T-B-363

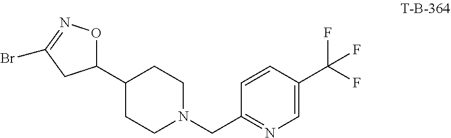 [SMILES: FC(F)(F)c1ccc(CN2CCC(C3CC(Br)=NO3)CC2)nc1]

T-B-364

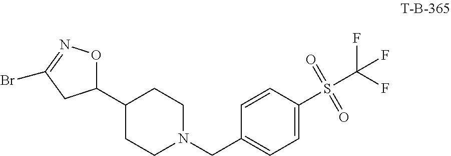 [SMILES: O=S(=O)(c1ccc(CN2CCC(C3CC(Br)=NO3)CC2)cc1)C(F)(F)F]

T-B-365

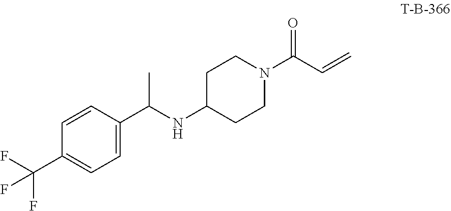 [SMILES: C=CC(=O)N1CCC(NC(C)c2ccc(C(F)(F)F)cc2)CC1]

T-B-366

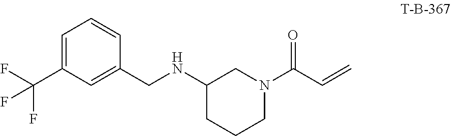 [SMILES: C=CC(=O)N1CCCC(NCc2cccc(C(F)(F)F)c2)C1]

T-B-367

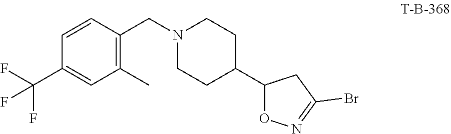 [SMILES: Cc1cc(C(F)(F)F)ccc1CN1CCC(C2CC(Br)=NO2)CC1]

T-B-368

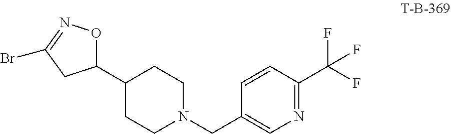 [SMILES: FC(F)(F)c1ccc(CN2CCC(C3CC(Br)=NO3)CC2)cn1]

T-B-369

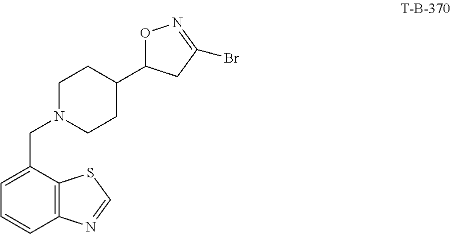 [SMILES: BrC1=NOC(C2CCN(Cc3cccc4ncsc34)CC2)C1]

T-B-370

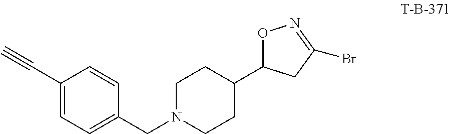 [SMILES: C#Cc1ccc(CN2CCC(C3CC(Br)=NO3)CC2)cc1]

T-B-371

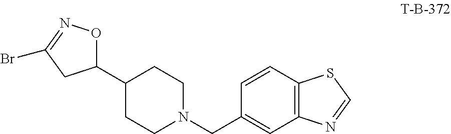 [SMILES: BrC1=NOC(C2CCN(Cc3ccc4scnc4c3)CC2)C1]

T-B-372

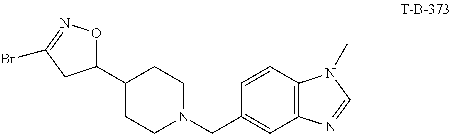 [SMILES: Cn1cnc2cc(CN3CCC(C4CC(Br)=NO4)CC3)ccc21]

T-B-373

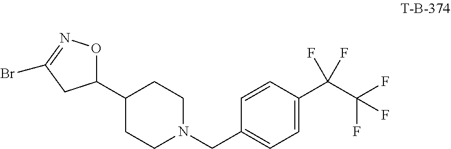 [SMILES: FC(F)(F)C(F)(F)c1ccc(CN2CCC(C3CC(Br)=NO3)CC2)cc1]

T-B-374

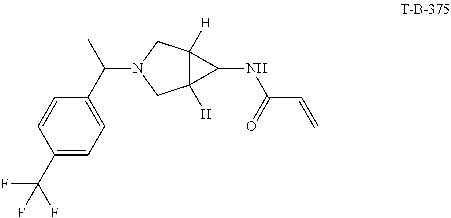 [SMILES: C=CC(=O)NC1C2CN(C(C)c3ccc(C(F)(F)F)cc3)CC21]

T-B-375

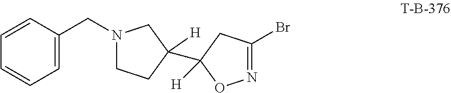 [SMILES: BrC1=NOC(C2CCN(Cc3ccccc3)C2)C1]

T-B-376

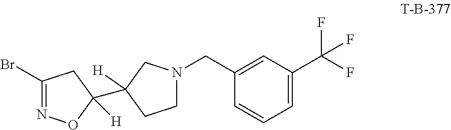 [SMILES: FC(F)(F)c1cccc(CN2CCC(C3CC(Br)=NO3)C2)c1]

T-B-377

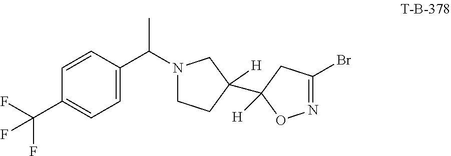 [SMILES: CC(c1ccc(C(F)(F)F)cc1)N1CCC(C2CC(Br)=NO2)C1]

T-B-378

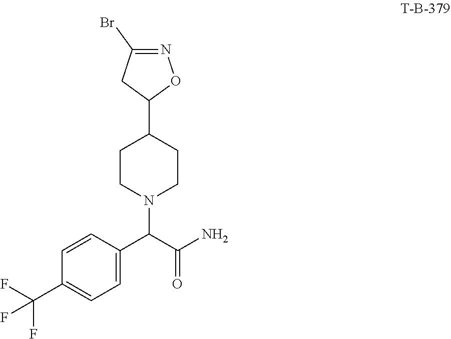 [SMILES: NC(=O)C(c1ccc(C(F)(F)F)cc1)N1CCC(C2CC(Br)=NO2)CC1]

T-B-379

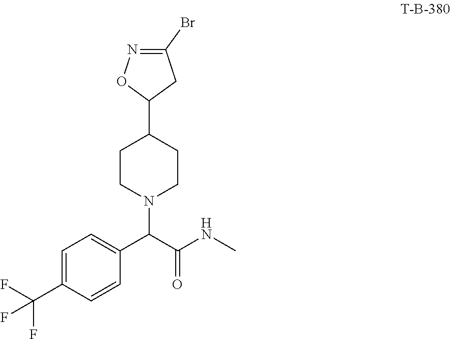 [SMILES: CNC(=O)C(c1ccc(C(F)(F)F)cc1)N1CCC(C2CC(Br)=NO2)CC1]

T-B-380

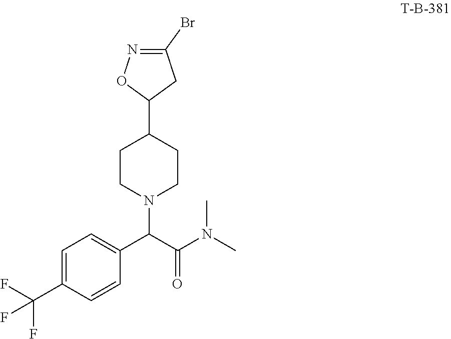 [SMILES: CN(C)C(=O)C(c1ccc(C(F)(F)F)cc1)N1CCC(C2CC(Br)=NO2)CC1]

T-B-381

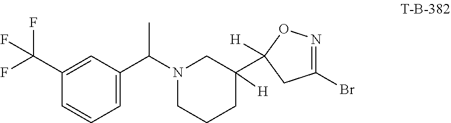 [SMILES: CC(c1cccc(C(F)(F)F)c1)N1CCCC(C2CC(Br)=NO2)C1]

T-B-382

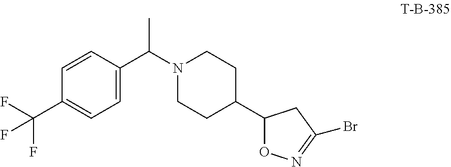 [SMILES: CC(c1ccc(C(F)(F)F)cc1)N1CCC(C2CC(Br)=NO2)CC1]

T-B-385

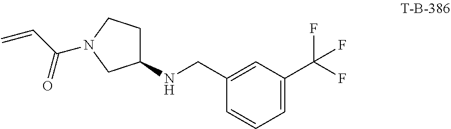 [SMILES: C=CC(=O)N1CC[C@@H](NCc2cccc(C(F)(F)F)c2)C1]

T-B-386

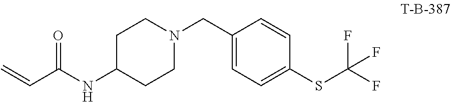 [SMILES: C=CC(=O)NC1CCN(Cc2ccc(SC(F)(F)F)cc2)CC1]

T-B-387

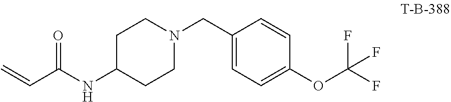 [SMILES: C=CC(=O)NC1CCN(Cc2ccc(OC(F)(F)F)cc2)CC1]

T-B-388

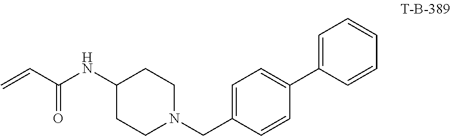 [SMILES: C=CC(=O)NC1CCN(Cc2ccc(-c3ccccc3)cc2)CC1]

T-B-389

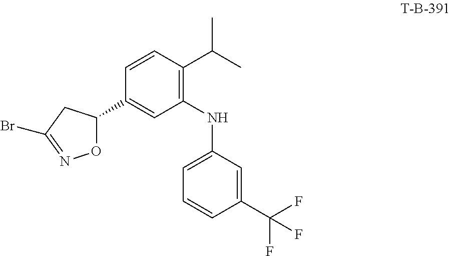 [SMILES: CC(C)c1ccc([C@H]2CC(Br)=NO2)cc1Nc1cccc(C(F)(F)F)c1]

T-B-391

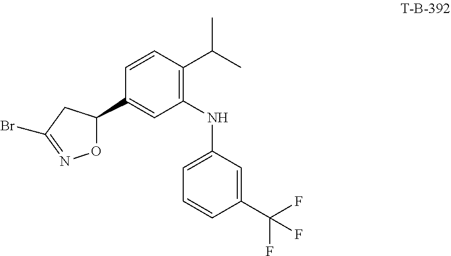 [SMILES: CC(C)c1ccc([C@@H]2CC(Br)=NO2)cc1Nc1cccc(C(F)(F)F)c1]

T-B-392

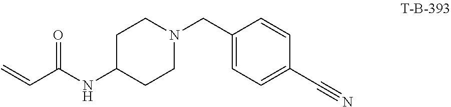 [SMILES: C=CC(=O)NC1CCN(Cc2ccc(C#N)cc2)CC1]

T-B-393

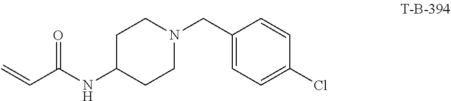 [SMILES: C=CC(=O)NC1CCN(Cc2ccc(Cl)cc2)CC1]

T-B-394

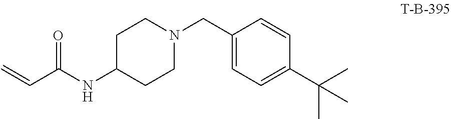 [SMILES: C=CC(=O)NC1CCN(Cc2ccc(C(C)(C)C)cc2)CC1]

T-B-395

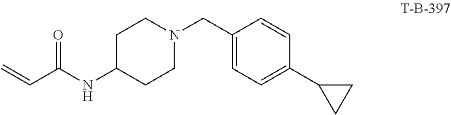 [SMILES: C=CC(=O)NC1CCN(Cc2ccc(C3CC3)cc2)CC1]

T-B-397

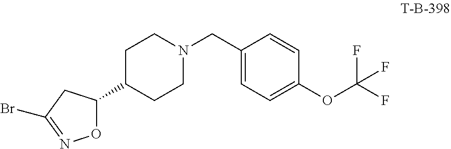 [SMILES: FC(F)(F)Oc1ccc(CN2CCC([C@H]3CC(Br)=NO3)CC2)cc1]

T-B-398

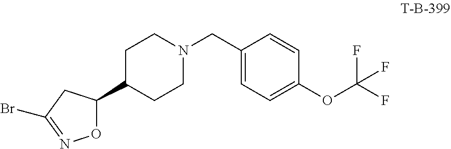 [SMILES: FC(F)(F)Oc1ccc(CN2CCC([C@@H]3CC(Br)=NO3)CC2)cc1]

T-B-399

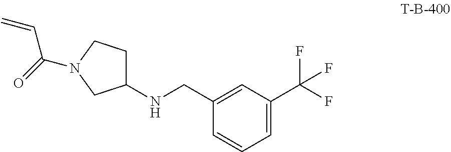 [SMILES: C=CC(=O)N1CCC(NCc2cccc(C(F)(F)F)c2)C1]

T-B-400

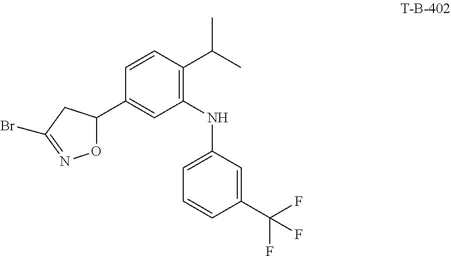 [SMILES: CC(C)c1ccc(C2CC(Br)=NO2)cc1Nc1cccc(C(F)(F)F)c1]

T-B-402

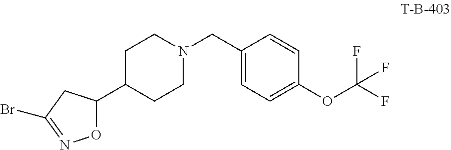 [SMILES: FC(F)(F)Oc1ccc(CN2CCC(C3CC(Br)=NO3)CC2)cc1]

T-B-403

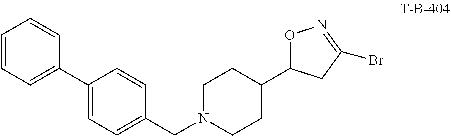 [SMILES: BrC1=NOC(C2CCN(Cc3ccc(-c4ccccc4)cc3)CC2)C1]

T-B-404

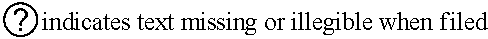

### 3. TEAD Inhibitors of Formulae C, and C-1 to C-85

In certain embodiments, a TEAD inhibitor is a compound of Formula C:

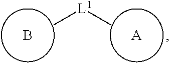

or a pharmaceutically acceptable salt thereof, wherein
L¹ is a covalent bound, or a C₁₋₆ bivalent straight or branched hydrocarbon chain wherein 1, 2, or 3 methylene units of the chain are independently and optionally replaced with —N(R)—, —O—, or —C(O)—;
Ring A is selected from

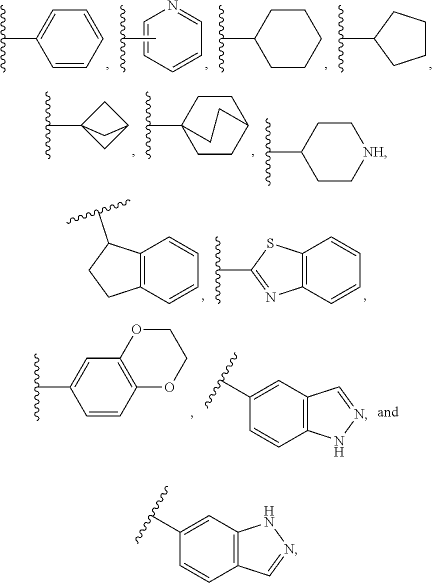

each of which is optionally substituted;
Ring B is selected from

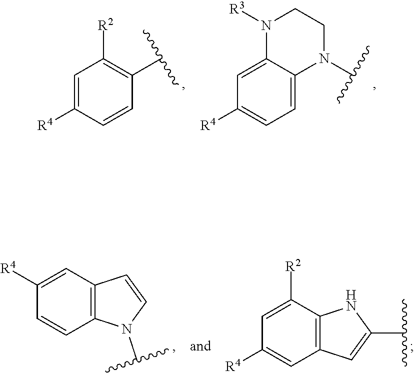

each R² is independently selected from —OR, —C(O)NR₂, optionally substituted —C₁₋₆ aliphatic,

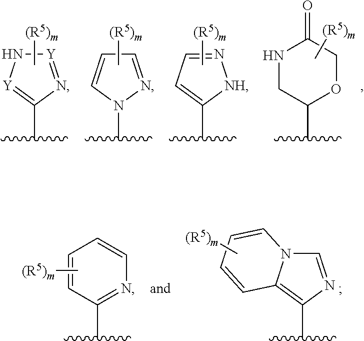

each Y is independently N or CR⁵;
R³ is H, —C(O)R, or optionally substituted —C₁₋₆ aliphatic;
each R⁴ is independently —S(O)₂NR₂, —S(O)₂R, —C(O)NR₂, —C(O)R, or optionally substituted —C₁₋₆ aliphatic;
each R⁵ is independently R, —CN, —C(O)R, —C(O)NR₂, or optionally substituted 5-6 membered heteroaryl having 1-2 heteroatoms independently selected from nitrogen, oxygen, or sulfur;
each m is independently 0, 1, or 2; and
each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

As defined generally above, L¹ is a covalent bound, or a C₁₋₆ bivalent straight or branched hydrocarbon chain wherein 1, 2, or 3 methylene units of the chain are independently and optionally replaced with —N(R)—, —O—, or —C(O)—.

In some embodiments, L¹ is a covalent bond.

In some embodiments, L¹ is C₁₋₆ bivalent straight or branched hydrocarbon chain wherein 1, 2, or 3 methylene units of the chain are independently and optionally replaced with —N(R)—.

In some embodiments, L¹ is C₁₋₆ bivalent straight or branched hydrocarbon chain wherein 1, 2, or 3 methylene units of the chain are independently and optionally replaced with —O—.

In some embodiments, L¹ is C₁₋₆ bivalent straight or branched hydrocarbon chain wherein 1, 2, or 3 methylene units of the chain are independently and optionally replaced with —C(O)—.

In some embodiments, L¹ is —NH—. In some embodiments, L¹ is —NH—CH₂—. In some embodiments, L¹ is —NH—CH₂—CH₂—. In some embodiments, L¹ is —CH₂—. In some embodiments, L is

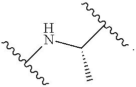 [SMILES: *N[C@H](*)C]

In some embodiments, L¹ is

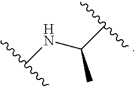 [SMILES: *N[C@@H](*)C]

In some embodiments, L is

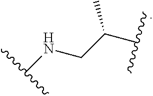 [SMILES: *NC[C@@H](*)C]

In some embodiments, L¹ is

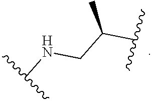 [SMILES: *NC[C@H](*)C]

In some embodiments, L¹ is —CH═CH—. In some embodiments, L¹ is

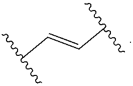 [SMILES: */C=C/*]

In some embodiments, L is

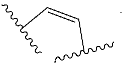 [SMILES: */C=C\*]

In some embodiments, L¹ is —NH—C(O)—.

In some embodiments, L¹ is selected from those depicted in Table C, below.

As defined generally above, Ring A is selected from

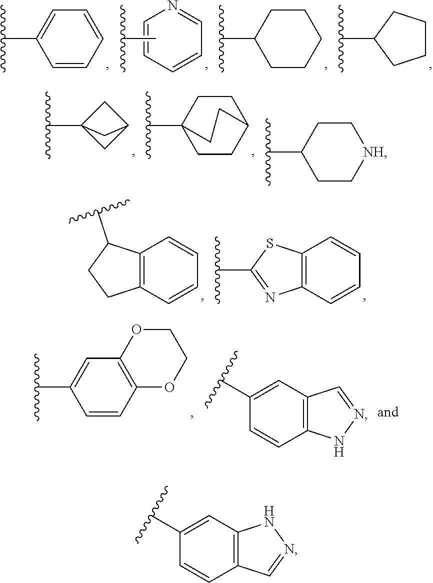

each of which is optionally substituted.

In some embodiments, Ring A is optionally substituted

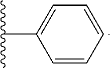 [SMILES: *c1ccccc1]

In some embodiments, Ring A is optionally substituted

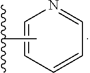

In some embodiments, Ring A is optionally substituted

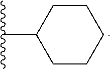 [SMILES: *C1CCCCC1]

In some embodiments, Ring A is optionally substituted

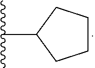 [SMILES: *C1CCCC1]

In some embodiments, Ring A is optionally substituted

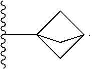 [SMILES: *C12CC(C1)C2]

In some embodiments, Ring A is optionally substituted

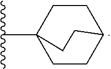 [SMILES: *C12CCC(CC1)CC2]

In some embodiments, Ring A is optionally substituted

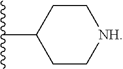 [SMILES: *C1CCNCC1]

In some embodiments, Ring A is optionally substituted

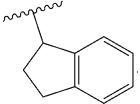 [SMILES: *C1CCc2ccccc21]

In some embodiments, Ring A is optionally substituted

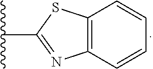 [SMILES: *c1nc2ccccc2s1]

In some embodiments, Ring A is optionally substituted

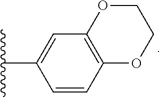 [SMILES: *c1ccc2c(c1)OCCO2]

In some embodiments, Ring A is optionally substituted

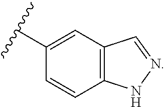 [SMILES: *c1ccc2[nH]ncc2c1]

In some embodiments, Ring A is optionally substituted

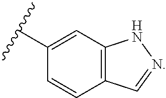 [SMILES: *c1ccc2cn[nH]c2c1]

In some embodiments, Ring A is selected from

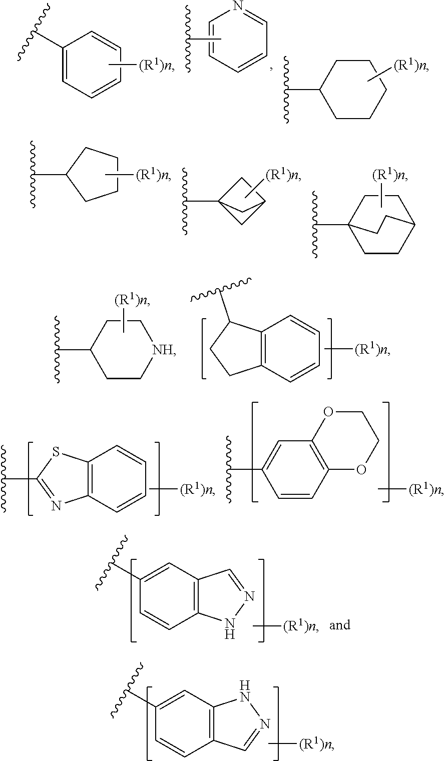

wherein each R¹ is independently R, halogen, —CN, —C(O)R, —C(O)NR₂, —OR, —SR, —S(O)₂NR₂, or —S(O)₂R, and each n is independently 0, 1, 2, or 3, wherein each R is independently as defined herein and as described in embodiments herein.

In some embodiments, R¹ is R. In some embodiments, R¹ is halogen. In some embodiments, R¹ is —CN. In some embodiments, R¹ is —C(O)R. In some embodiments, R¹ is —C(O)NR₂. In some embodiments, R¹ is —OR. In some embodiments, R¹ is —SR. In some embodiments, R¹ is —S(O)₂NR₂. In some embodiments, R¹ is —S(O)₂R.

In some embodiments, each R¹ is independently H, halogen, —C₁₋₆ aliphatic optionally substituted by 1-6 halogen, 3-8 membered saturated or partially unsaturated monocyclic carbocyclyl optionally substituted by 1-6 halogen, or 3-8 membered saturated or partially unsaturated monocyclic heterocyclyl having 1-2 heteroatoms independently selected from nitrogen, oxygen, or sulfur optionally substituted by 1-6 halogen.

In some embodiments, each R¹ is independently H, —CF₃, —C(O)NH₂, —CH₃, —CH₂CH₃, —OCH₃, —CHF₂, —OCF₃, —OCHF₂, —SCF₃, —Cl, —S(O)₂—NH₂, —OCH₂CH₃, —F, —C(O)NHCH₃, —CN, —S(O)₂—CH₃, —OCH(CH₃)₂, —CH(CH₃)₂, —C(CH₃)₃, —CH₂OH,

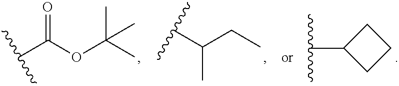

In some embodiments, each R¹ is independently selected from those depicted in Table C, below.

In some embodiments, n is 0. In some embodiments, n is 1. In some embodiments, n is 2. In some embodiments, n is 3.

In some embodiments, Ring A is selected from

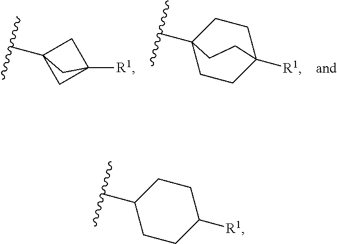

wherein each of R¹ is as defined above and described in embodiments herein, both singly and in combination.

In some embodiments, Ring A is selected from

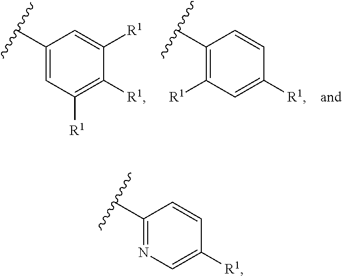

wherein each R¹ is as defined above and as described in embodiments herein, both singly and in combination.

In some embodiments, Ring A is

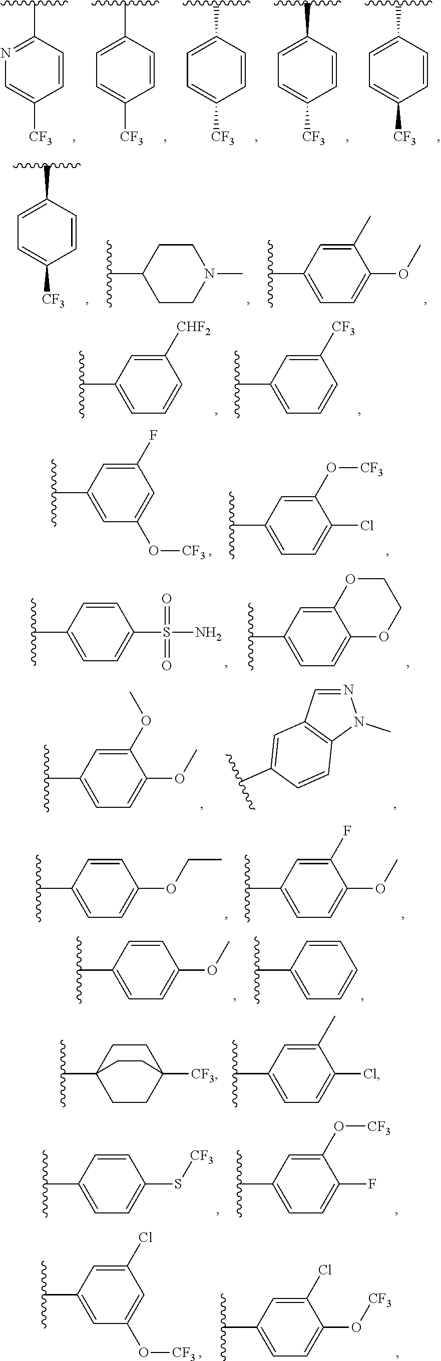

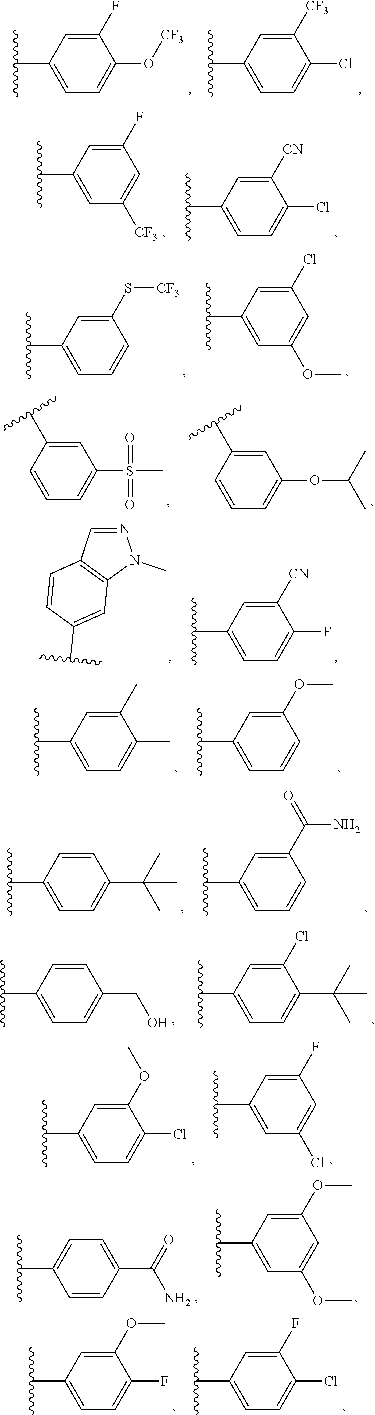

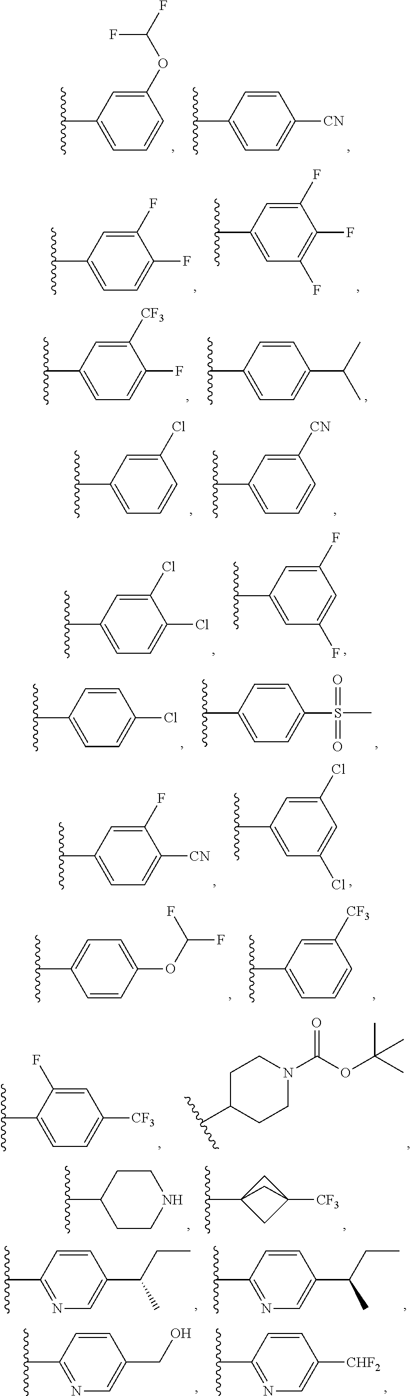

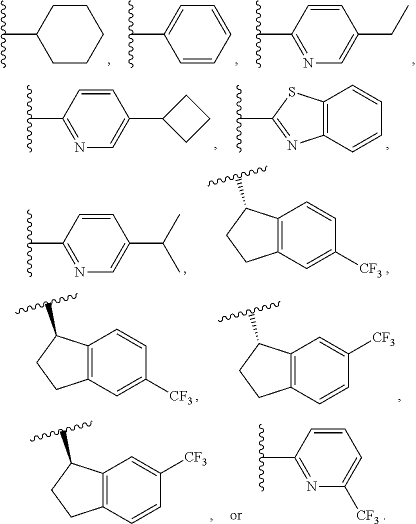

In some embodiments, Ring A is selected from those depicted in Table C, below.

As defined generally above, Ring B is selected from

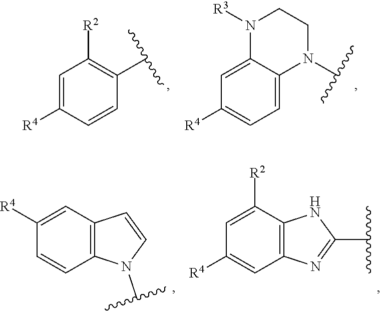

wherein each of R², R³, and R⁴ is as defined herein and as described in embodiments herein, both singly and in combination.

In some embodiments, Ring B is

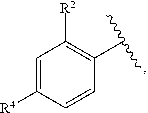

wherein each of R² and R⁴ is as defined above and as described in embodiments herein, both singly and in combination.

In some embodiments, Ring B is

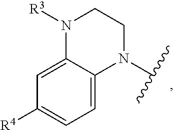

wherein each of R³ and R⁴ is as defined above and as described in embodiments herein, both singly and in combination.

In some embodiments, Ring B is

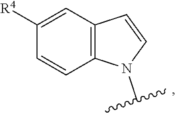

wherein R⁴ is as defined above and as described in embodiments herein.

In some embodiments, Ring B is

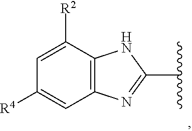

wherein each of R² and R⁴ is as defined above and as described in embodiments herein, both singly and in combination.

In some embodiments, Ring B is

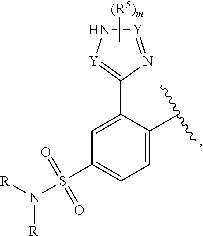

wherein each of R, Y, m, and R⁵ is as defined above and as described in embodiments herein, both singly and in combination. In some embodiments, Ring B is

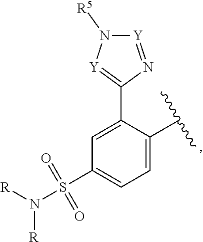

wherein each of Y, R, and R⁵ is as defined above and as described in embodiments herein, both singly and in combination. In some embodiments, Ring B is

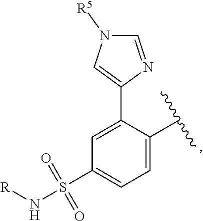

wherein each of R and R⁵ is as defined above and as described in embodiments herein, both singly and in combination.

In some embodiments, Ring B is

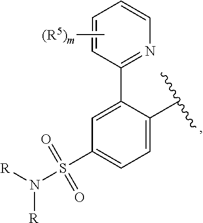

wherein each of m, R, and R⁵ is as defined above and as described in embodiments herein, both singly and in combination. In some embodiments, Ring B is

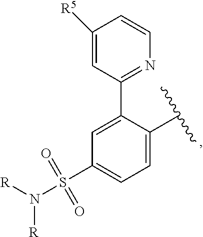

wherein each of R and R⁵ is as defined above and as described in embodiments herein, both singly and in combination. In some embodiments, Ring B is

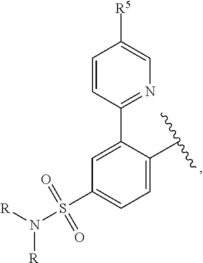

wherein each of R and R⁵ is as defined above and as described in embodiments herein, both singly and in combination. In some embodiments, Ring B is

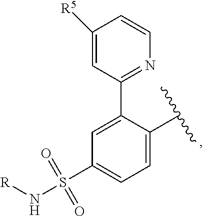

wherein each of R and R⁵ is as defined above and as described in embodiments herein, both singly and in combination. In some embodiments, Ring B is

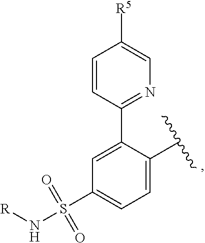

wherein each of R and R⁵ is as defined above and as described in embodiments herein, both singly and in combination.

In some embodiments, Ring B is

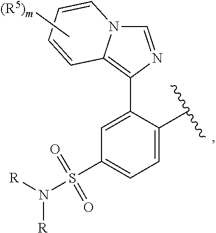

wherein each of m, R, and R⁵ is as defined above and as described in embodiments herein, both singly and in combination. In some embodiments, Ring B is

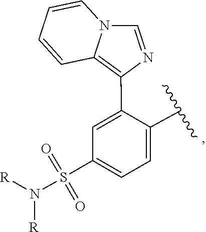

wherein each R is independently as defined above and described in embodiments herein. In some embodiments, Ring B is

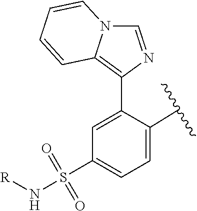

wherein R is as defined above and as described in embodiments herein.

In some embodiments, Ring B is

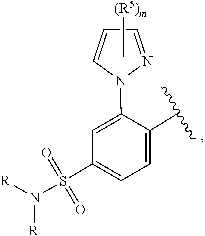

wherein each of m, R, and R⁵ is as defined above and as described in embodiments herein, both singly and in combination. In some embodiments, Ring B is

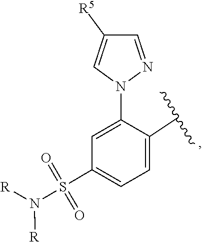

wherein each of R and R⁵ is as defined above and as described in embodiments herein, both singly and in combination. In some embodiments, Ring B is

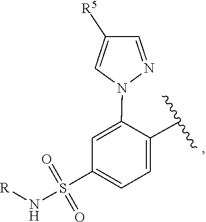

wherein each of R and R⁵ is as defined above and as described in embodiments herein, both singly and in combination.

In some embodiments, Ring B is selected from those depicted in Table C, below.

As defined generally above, each R² is independently selected from —OR, —C(O)NR₂, optionally substituted —C₁₋₆ aliphatic,

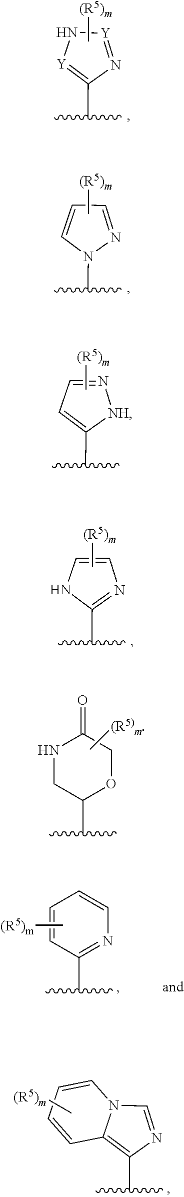

wherein each of Y, m, and R⁵ is as defined herein and as described in embodiments herein, both singly and in combination.

In some embodiments, R² is —OR. In some embodiments, R² is —C(O)NR₂. In some embodiments, R² is optionally substituted —C₁₋₆ aliphatic. In some embodiments, R² is

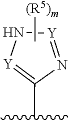

In some embodiments, R² is

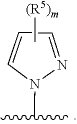

In some embodiments, R² is

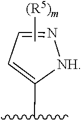

In some embodiments, R² is

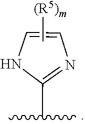

In some embodiments, R² is

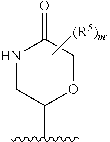

In some embodiments, R² is

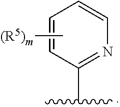

In some embodiments, R² is

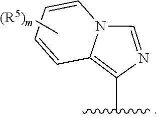

In some embodiments, R² is

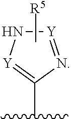

In some embodiments, R² is

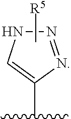

In some embodiments, R² is

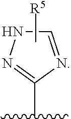

In some embodiments, R² is

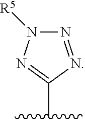

In some embodiments, R² is

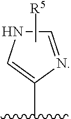

In some embodiments, R² is

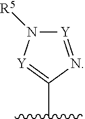

In some embodiments, R² is

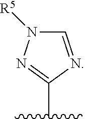

In some embodiments, R² is

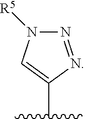

In some embodiments, R² is

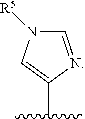

In some embodiments, R² is

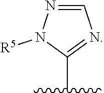

In some embodiments, R² is

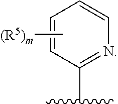

In some embodiments, R² is

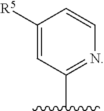

In some embodiments, R² is

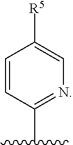

In some embodiments, R² is

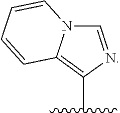 [SMILES: *c1ncn2ccccc12]

In some embodiments, R² is

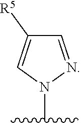

In some embodiments, R² is selected from:

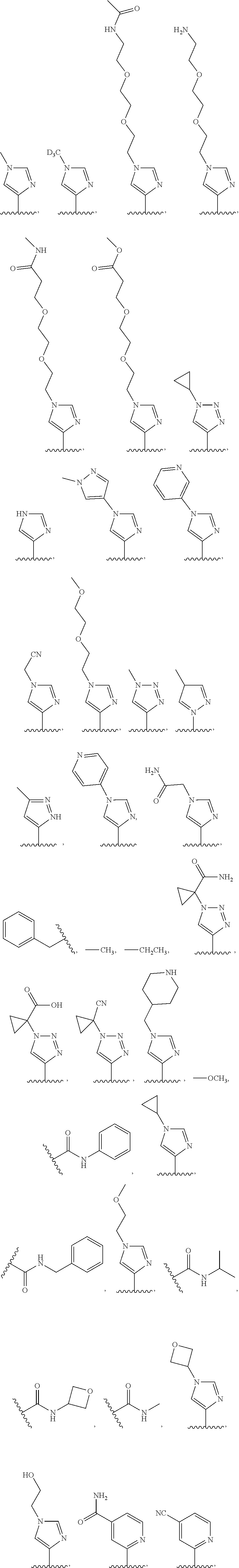

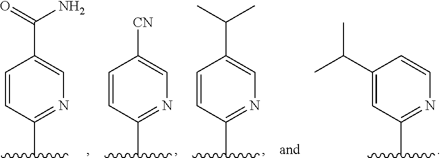

In some embodiments, R² is selected from

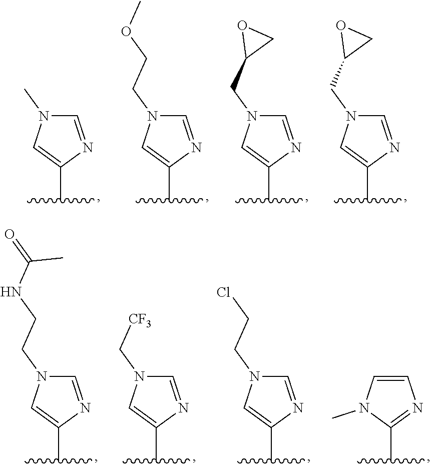

### and —OCH₃.

In some embodiments, R² is selected from those depicted in Table C, below.

As defined generally above, each Y is independently N or CR⁵.

In some embodiments, Y is N. In some embodiments, Y is CR⁵. In some embodiments, Y is CH.

In some embodiments, both Y are N. In some embodiments, both Y are CR⁵. In some embodiments, one Y is N, and the other Y is CR⁵. In some embodiments, both Y are CH. In some embodiments, one Y is N, and the other Y is CH.

In some embodiments, Y is selected from those depicted in Table C, below.

As defined generally above, R³ is —H, —C(O)R, or optionally substituted —C₁₋₆ aliphatic, wherein R is as defined herein and described in embodiments herein.

In some embodiments, R³ is —H.

In some embodiments, R³ is —C(O)R.

In some embodiments, R³ is optionally substituted —C₁₋₆ aliphatic.

In some embodiments, R³ is selected from H, —CH₃, —CH₂CH₃, —C(O)CH₃, and

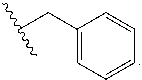 [SMILES: *Cc1ccccc1]

In some embodiments, R³ is selected from those depicted in Table C, below.

As defined generally above, each R⁴ is independently —S(O)₂NR₂, —S(O)₂R, —C(O)NR₂, —C(O)R, or optionally substituted —C₁₋₆ aliphatic, wherein each R is independently as defined herein and as described in embodiments herein.

In some embodiments, R⁴ is —S(O)₂NR₂.

In some embodiments, R⁴ is —S(O)₂R.

In some embodiments, R⁴ is —C(O)NR₂.

In some embodiments, R⁴ is —C(O)R.

In some embodiments, R⁴ is -optionally substituted —C₁₋₆ aliphatic.

In some embodiments, R⁴ is selected from

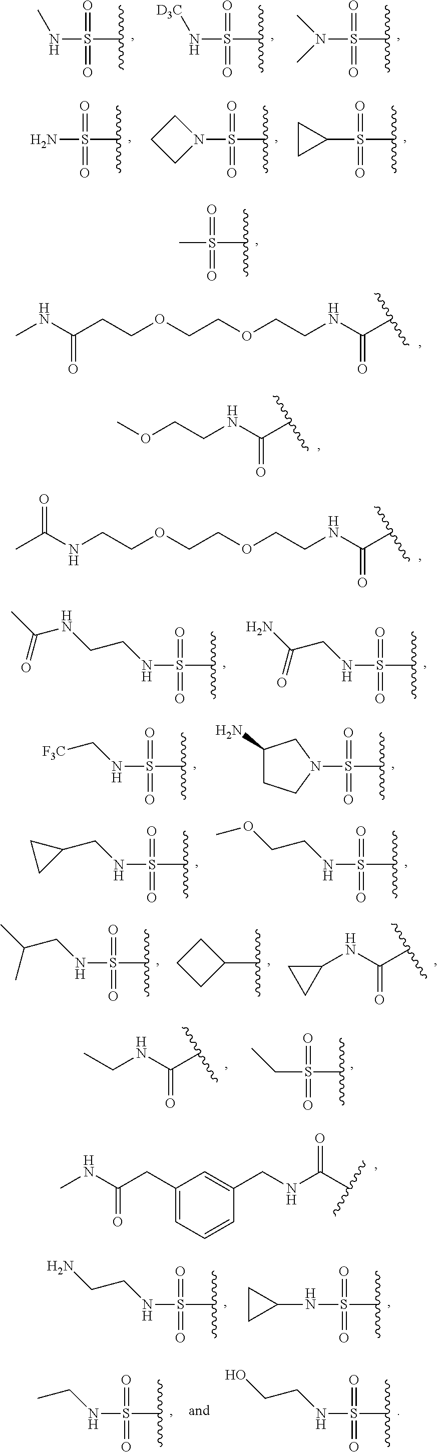

In some embodiments, R⁴ is selected from:

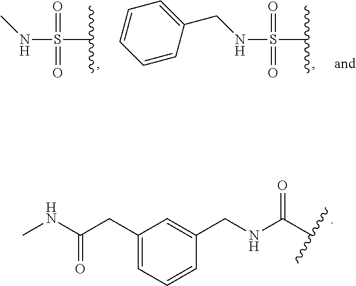

In some embodiments, R⁴ is selected from those depicted in Table C, below.

As defined generally above, each R⁵ is independently R, —CN, —C(O)R, —C(O)NR₂, or optionally substituted 5-6 membered heteroaryl having 1-2 heteroatoms independently selected from nitrogen, oxygen, or sulfur, wherein each R is independently as defined herein and as described in embodiments herein.

In some embodiments, R⁵ is R.

In some embodiments, R⁵ is —CN.

In some embodiments, R⁵ is —C(O)R.

In some embodiments, R⁵ is —C(O)NR₂.

In some embodiments, R⁵ is optionally substituted 5-6 membered heteroaryl having 1-2 heteroatoms independently selected from nitrogen, oxygen, or sulfur.

In some embodiments, each R⁵ is independently selected from: H, —CH₃, —CD₃,

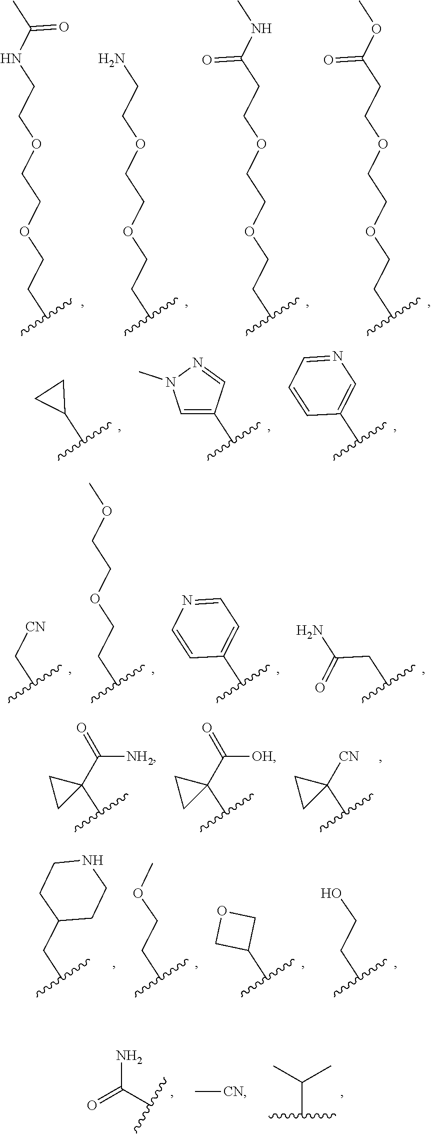

—CH₂CH₃, —C(O)CH₃, —CH₂C(O)NHCH₃,

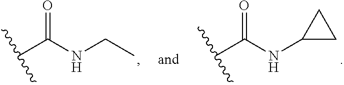

In some embodiments, each R⁵ is independently selected from: —CH₃, —CH₂CH₂OCH₃, —CH₂CF₃, —CH₂CH₂Cl,

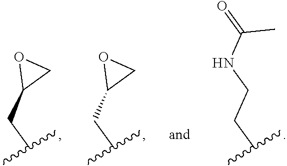

In some embodiments, R⁵ is selected from those depicted in Table C, below.

As defined generally above, each m is independently 0, 1, or 2.

In some embodiments, m is 0. In some embodiments, m is 1. In some embodiments, m is 2.

In some embodiments, m is selected from those depicted in Table C, below.

As defined generally above, each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

In some embodiments, R is H.

In some embodiments, R is optionally substituted —C₁₋₆ aliphatic. In some embodiments, R is unsubstituted —C₁₋₆ aliphatic. In some embodiments, R is —C₁₋₆ aliphatic substituted 1, 2, 3, 4, 5, or 6 times by -halogen, —CN, or —NO₂. In some embodiments, R is —C₁₋₆ aliphatic substituted 1, 2, 3, 4, 5, or 6 times by —F. In some embodiments, R is —C₁₋₃ aliphatic substituted 1, 2, 3, 4, 5, or 6 times by —F. In some embodiments, R is —CH₃. In some embodiments, R is —CH₂CH₃. In some embodiments, R is —CF₃. In some embodiments, R is —CHF₂.

In some embodiments, R is optionally substituted 3-8 membered saturated or partially unsaturated monocyclic carbocyclyl. In some embodiments, R is unsubstituted 3, 4, 5, 6, 7, or 8 membered saturated or partially unsaturated monocyclic carbocyclyl. In some embodiments, R is 3, 4, 5, 6, 7, or 8 membered saturated or partially unsaturated monocyclic carbocyclyl substituted 1, 2, 3, 4, 5, or 6 times by -halogen, —CN, —NO₂, or —C₁₋₆ aliphatic, wherein the —C₁₋₆ aliphatic is optionally substituted 1, 2, 3, 4, 5, or 6 times by -halogen, —CN, or —NO₂. In some embodiments, R is 3, 4, 5, 6, 7, or 8 membered saturated or partially unsaturated monocyclic carbocyclyl substituted 1, 2, 3, 4, 5, or 6 times by -halogen. In some embodiments, R is 3, 4, 5, 6, 7, or 8 membered saturated or partially unsaturated monocyclic carbocyclyl substituted 1, 2, 3, 4, 5, or 6 times by —C₁₋₆ aliphatic, wherein the —C₁₋₆ aliphatic is optionally substituted 1, 2, 3, 4, 5, or 6 times by -halogen. In some embodiments, R is 3, 4, 5, 6, 7, or 8 membered saturated or partially unsaturated monocyclic carbocyclyl substituted 1, 2, 3, 4, 5, or 6 times by —F. In some embodiments, R is 3, 4, 5, 6, 7, or 8 membered saturated or partially unsaturated monocyclic carbocyclyl substituted 1, 2, 3, 4, 5, or 6 times by —C₁₋₆ aliphatic, wherein the —C₁₋₆ aliphatic is optionally substituted 1, 2, 3, 4, 5, or 6 times by —F.

In some embodiments, R is optionally substituted 3-8 membered saturated or partially unsaturated monocyclic heterocyclyl having 1-2 heteroatoms independently selected from nitrogen, oxygen, or sulfur. In some embodiments, R is unsubstituted 3, 4, 5, 6, 7, or 8 membered saturated or partially unsaturated monocyclic heterocyclyl having 1-2 heteroatoms independently selected from nitrogen, oxygen, or sulfur. In some embodiments, R is 3, 4, 5, 6, 7, or 8 membered saturated or partially unsaturated monocyclic heterocyclyl having 1-2 heteroatoms independently selected from nitrogen, oxygen, or sulfur, which is substituted 1, 2, 3, 4, 5, or 6 times by -halogen, —CN, —NO₂, or —C₁₋₆ aliphatic, wherein the —C₁₋₆ aliphatic is optionally substituted 1, 2, 3, 4, 5, or 6 times by -halogen, —CN, or —NO₂. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by -halogen. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by —C₁₋₆ aliphatic, wherein the —C₁₋₆ aliphatic is optionally substituted 1, 2, 3, 4, 5, or 6 times by -halogen. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by —F. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by —C₁₋₆ aliphatic, wherein the —C₁₋₆ aliphatic is optionally substituted 1, 2, 3, 4, 5, or 6 times by —F.

In some embodiments, R is selected from

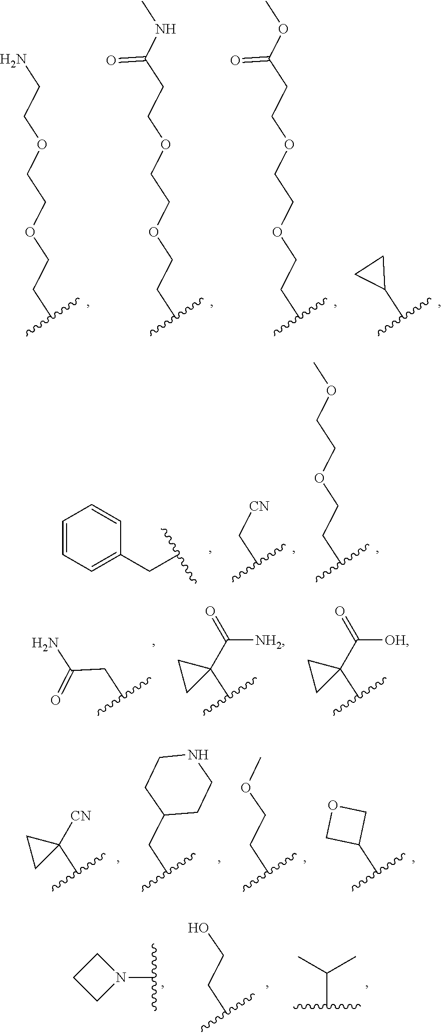

—CH₃, —CD₃, —CH₂CH₃, —CH₂C(O)NHCH₃,

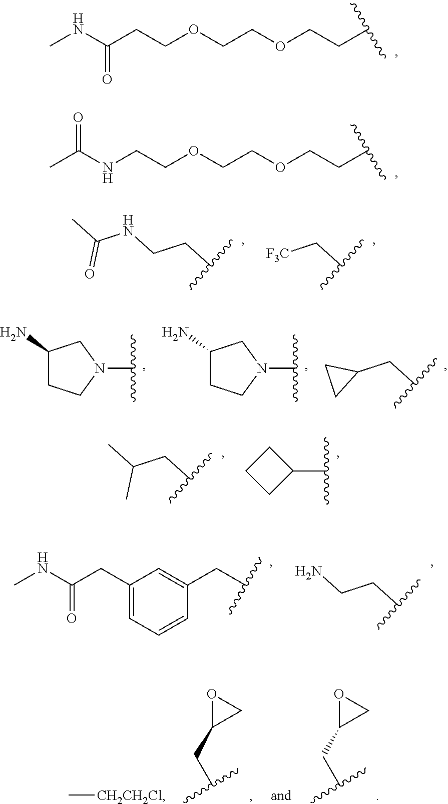

In some embodiments, R is selected from those depicted in Table C, below.

In some embodiments, a TEAD inhibitor is a compound selected from the following:

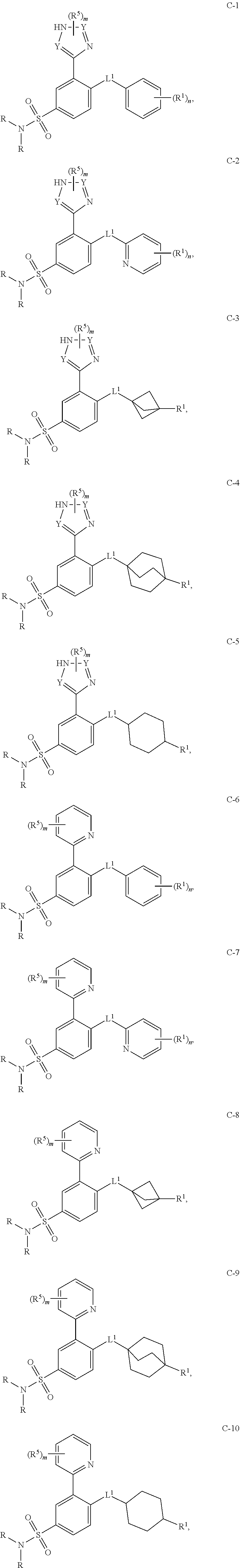

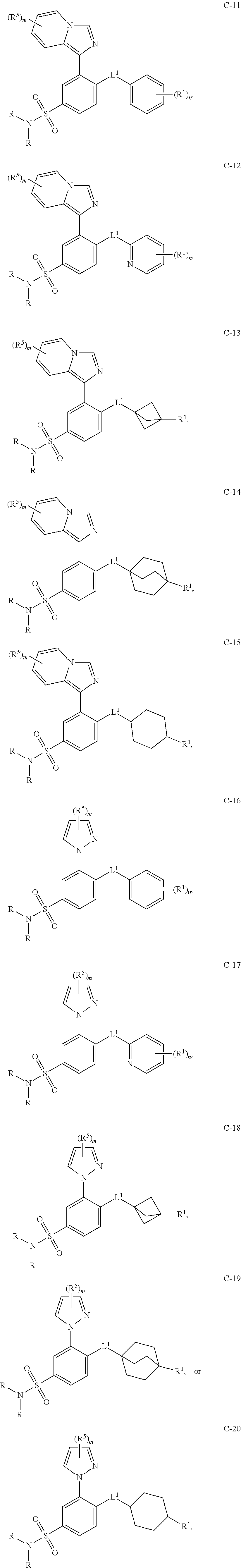

or a pharmaceutically acceptable salt thereof, wherein each of R, R¹, Y, L¹, m, n, and R is independently as defined and described in embodiments in the section of TEAD Inhibitors of Formulae C, and C-1 to C-85.

In some embodiments, a TEAD inhibitor is a compound selected from the following:

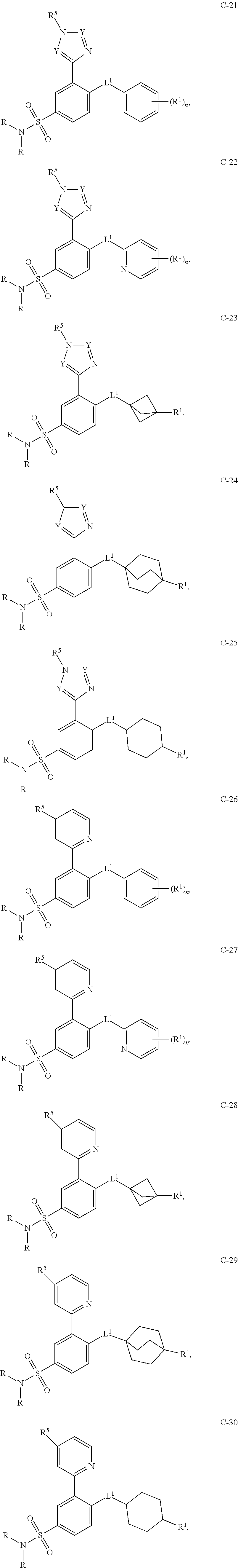

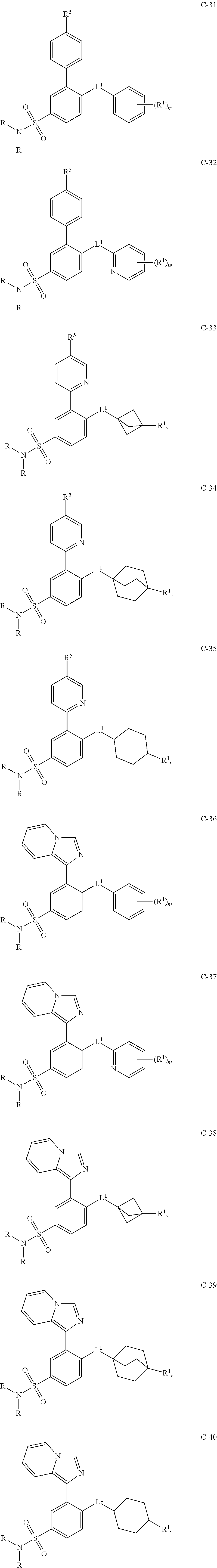

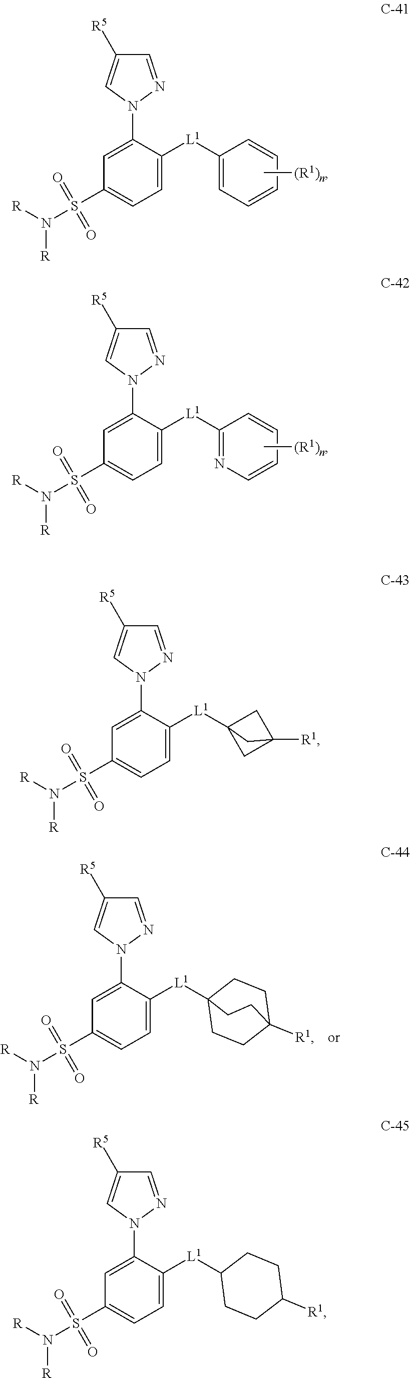

or a pharmaceutically acceptable salt thereof, wherein each of R, R¹, Y, L¹, n, and R⁵ is independently as defined and described in embodiments in the section of TEAD Inhibitors of Formulae C, and C-1 to C-85.

In some embodiments, a TEAD inhibitor is a compound selected from the following:

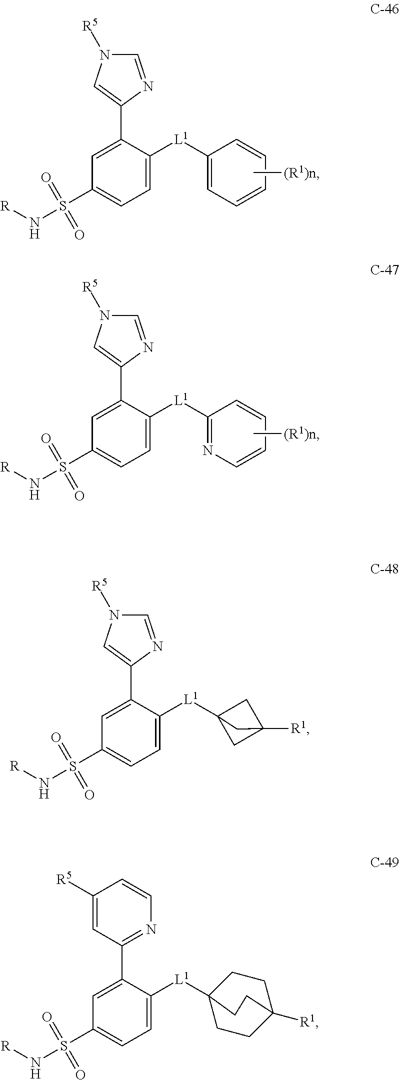

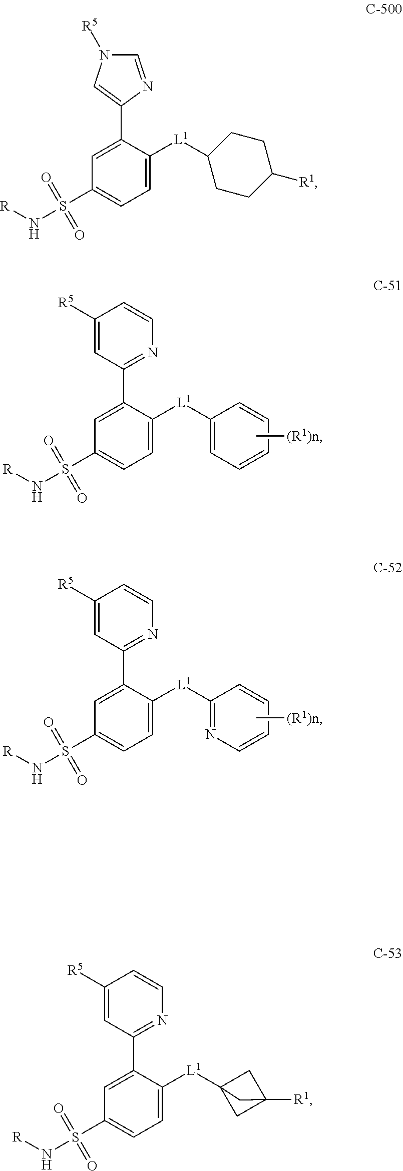

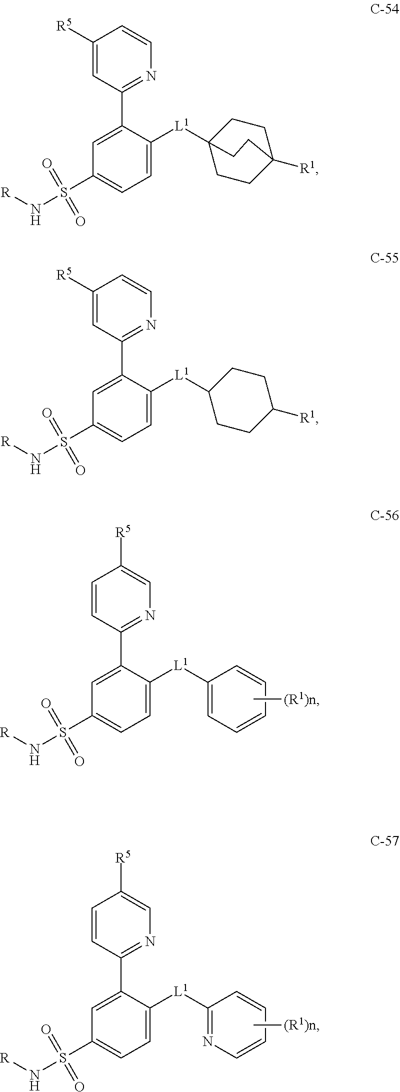

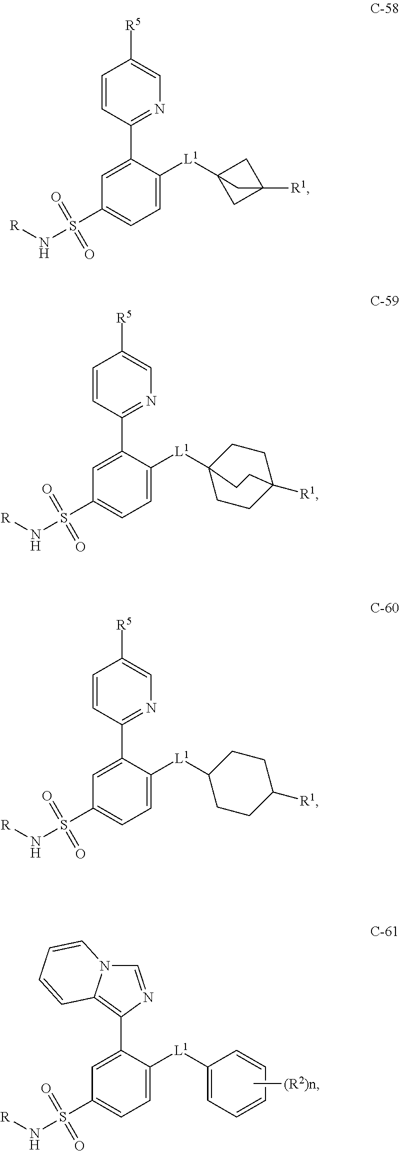

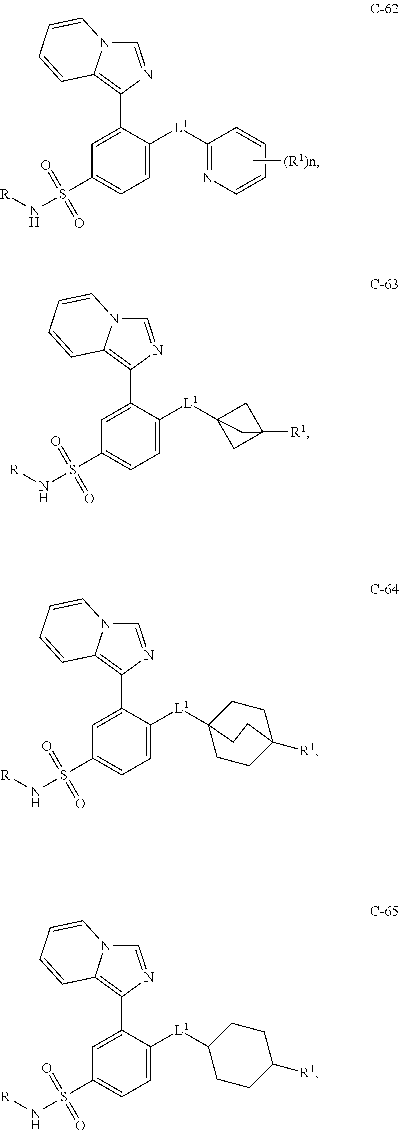

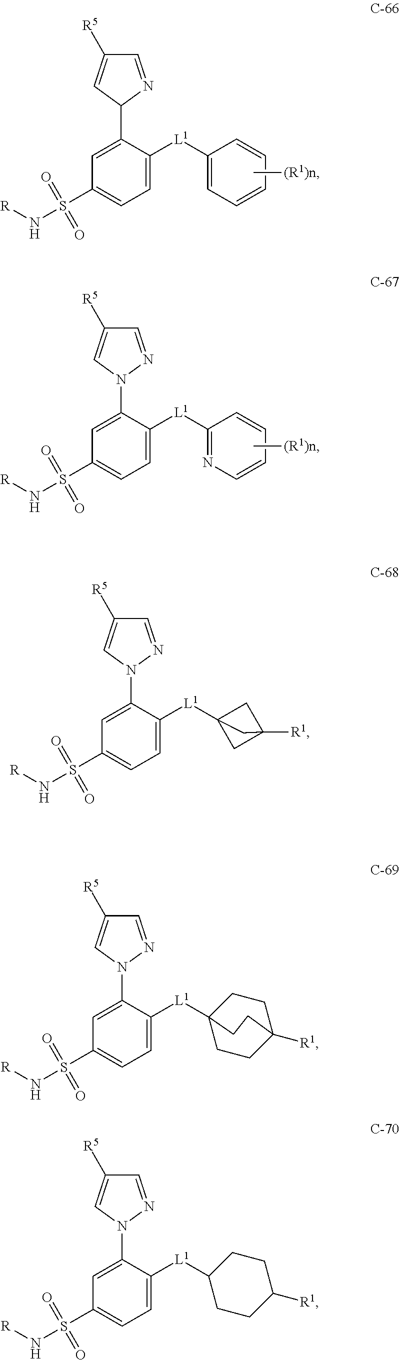

or a pharmaceutically acceptable salt thereof, wherein each of R, R¹, Y, L¹, n, and R⁵ is independently as defined and described in embodiments in the section of TEAD Inhibitors of Formulae C, and C-1 to C-85.

In some embodiments, a TEAD inhibitor is a compound selected from the following:

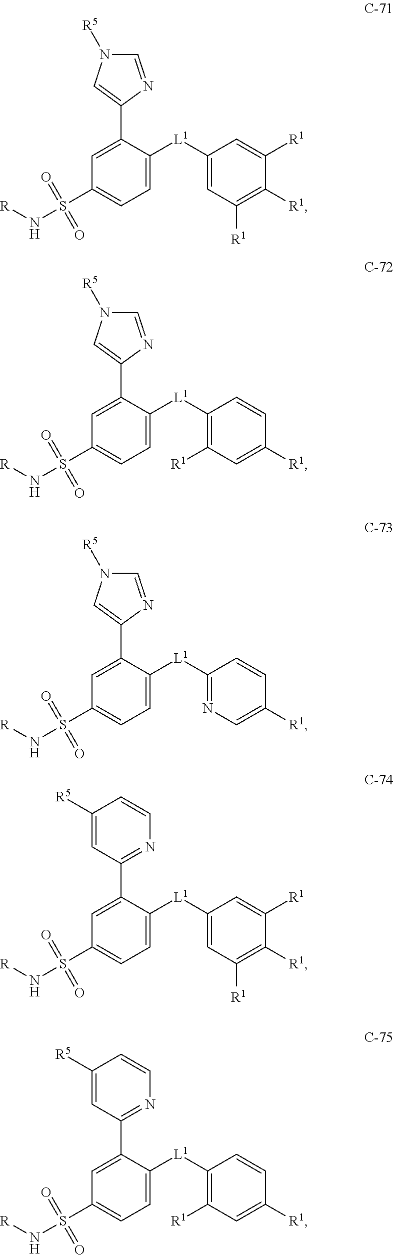

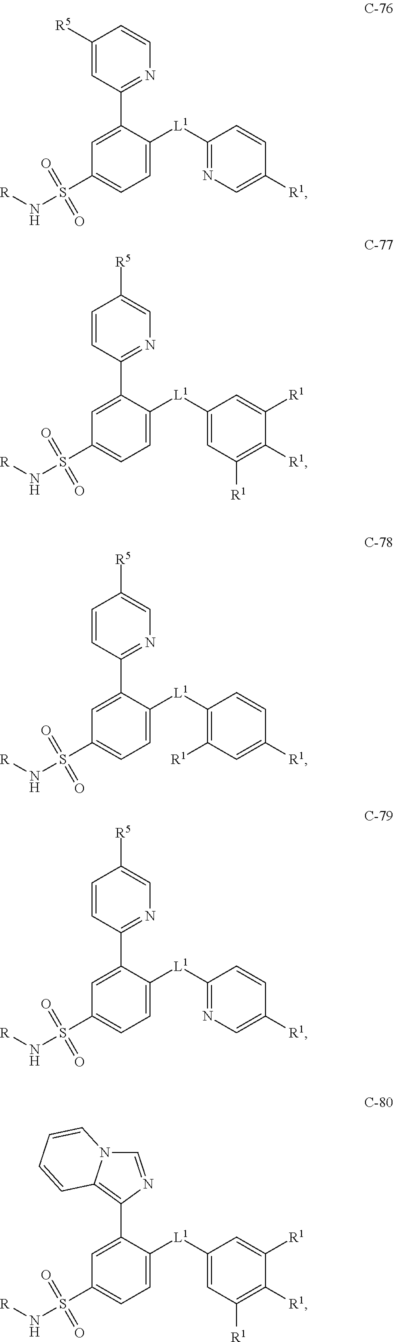

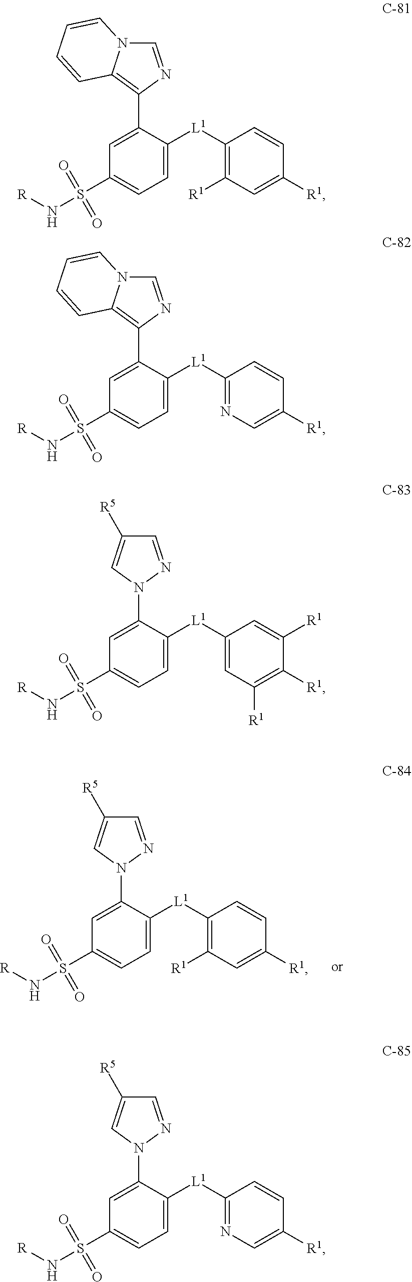

or a pharmaceutically acceptable salt thereof, wherein each of R, R¹, L¹, and R⁵ is independently as defined and described in embodiments in the section of TEAD Inhibitors of Formulae C, and C-1 to C-85.

In some embodiments, a TEAD inhibitor is selected from those listed in Table C, or a pharmaceutically acceptable salt thereof.

TABLE C
Exemplified TEAD Inhibitors

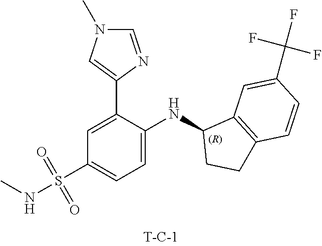

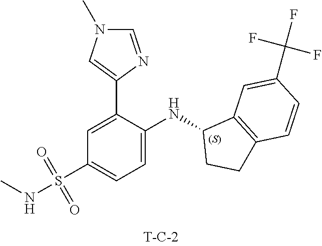

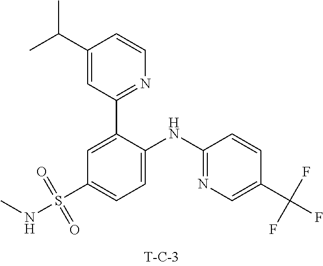 [SMILES: CNS(=O)(=O)c1ccc(Nc2ccc(C(F)(F)F)cn2)c(-c2cc(C(C)C)ccn2)c1]

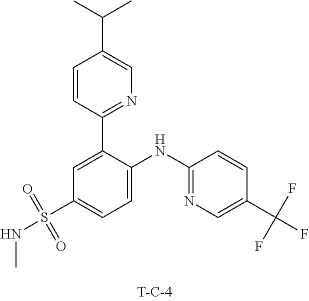 [SMILES: CNS(=O)(=O)c1ccc(Nc2ccc(C(F)(F)F)cn2)c(-c2ccc(C(C)C)cn2)c1]

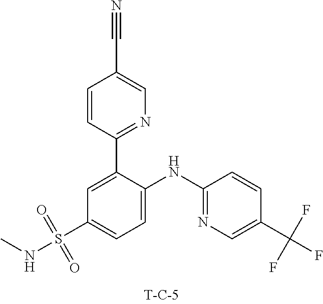 [SMILES: CNS(=O)(=O)c1ccc(Nc2ccc(C(F)(F)F)cn2)c(-c2ccc(C#N)cn2)c1]

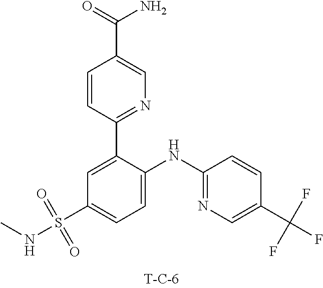 [SMILES: CNS(=O)(=O)c1ccc(Nc2ccc(C(F)(F)F)cn2)c(-c2ccc(C(N)=O)cn2)c1]

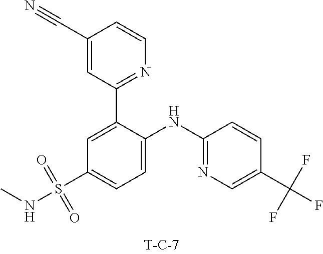 [SMILES: CNS(=O)(=O)c1ccc(Nc2ccc(C(F)(F)F)cn2)c(-c2cc(C#N)ccn2)c1]

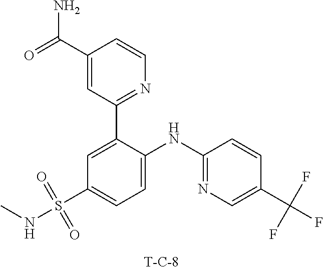 [SMILES: CNS(=O)(=O)c1ccc(Nc2ccc(C(F)(F)F)cn2)c(-c2cc(C(N)=O)ccn2)c1]

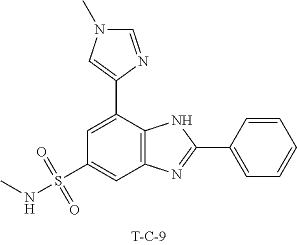 [SMILES: CNS(=O)(=O)c1cc(-c2cn(C)cn2)c2[nH]c(-c3ccccc3)nc2c1]

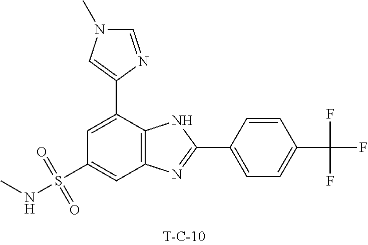 [SMILES: CNS(=O)(=O)c1cc(-c2cn(C)cn2)c2[nH]c(-c3ccc(C(F)(F)F)cc3)nc2c1]

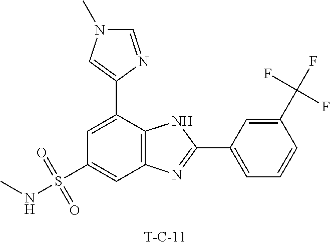 [SMILES: CNS(=O)(=O)c1cc(-c2cn(C)cn2)c2[nH]c(-c3cccc(C(F)(F)F)c3)nc2c1]

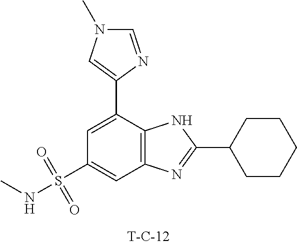 [SMILES: CNS(=O)(=O)c1cc(-c2cn(C)cn2)c2[nH]c(C3CCCCC3)nc2c1]

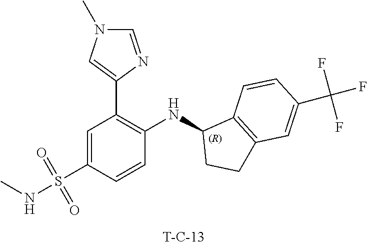

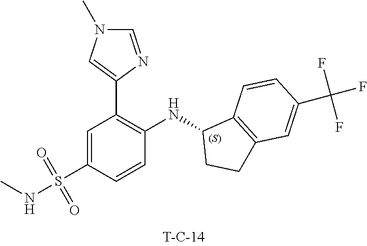

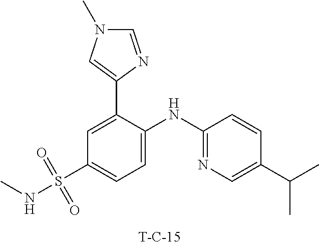 [SMILES: CNS(=O)(=O)c1ccc(Nc2ccc(C(C)C)cn2)c(-c2cn(C)cn2)c1]

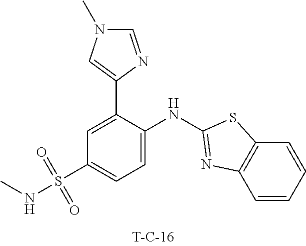 [SMILES: CNS(=O)(=O)c1ccc(Nc2nc3ccccc3s2)c(-c2cn(C)cn2)c1]

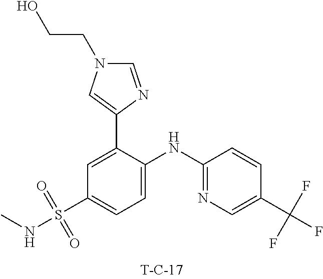 [SMILES: CNS(=O)(=O)c1ccc(Nc2ccc(C(F)(F)F)cn2)c(-c2cn(CCO)cn2)c1]

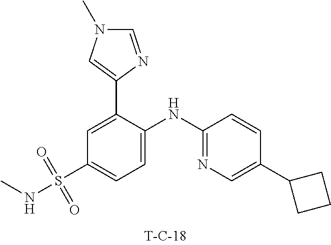 [SMILES: CNS(=O)(=O)c1ccc(Nc2ccc(C3CCC3)cn2)c(-c2cn(C)cn2)c1]

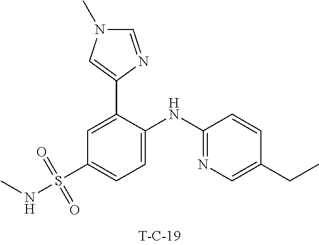 [SMILES: CCc1ccc(Nc2ccc(S(=O)(=O)NC)cc2-c2cn(C)cn2)nc1]

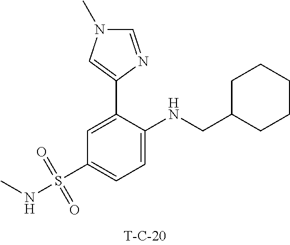 [SMILES: CNS(=O)(=O)c1ccc(NCC2CCCCC2)c(-c2cn(C)cn2)c1]

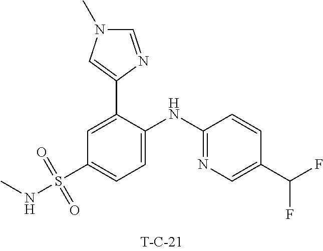 [SMILES: CNS(=O)(=O)c1ccc(Nc2ccc(C(F)F)cn2)c(-c2cn(C)cn2)c1]

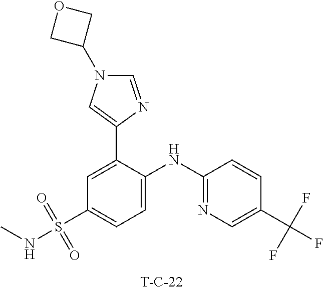 [SMILES: CNS(=O)(=O)c1ccc(Nc2ccc(C(F)(F)F)cn2)c(-c2cn(C3COC3)cn2)c1]

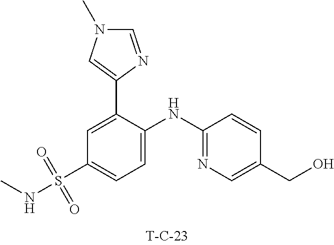 [SMILES: CNS(=O)(=O)c1ccc(Nc2ccc(CO)cn2)c(-c2cn(C)cn2)c1]

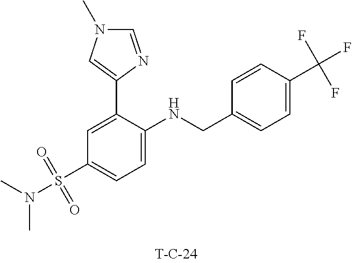 [SMILES: CN(C)S(=O)(=O)c1ccc(NCc2ccc(C(F)(F)F)cc2)c(-c2cn(C)cn2)c1]

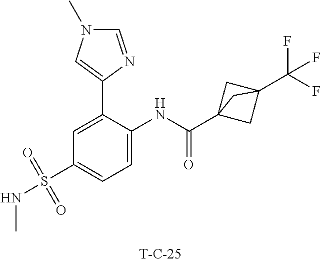 [SMILES: CNS(=O)(=O)c1ccc(NC(=O)C23CC(C(F)(F)F)(C2)C3)c(-c2cn(C)cn2)c1]

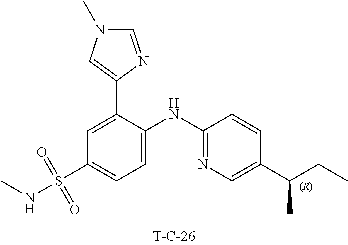

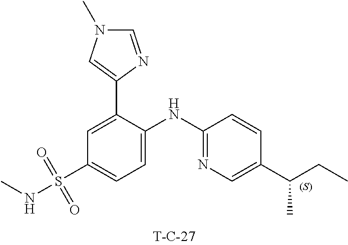

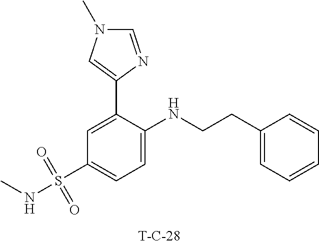 [SMILES: CNS(=O)(=O)c1ccc(NCCc2ccccc2)c(-c2cn(C)cn2)c1]

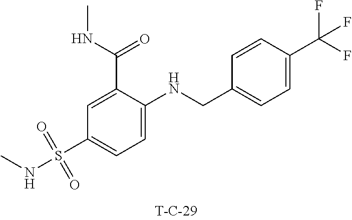 [SMILES: CNC(=O)c1cc(S(=O)(=O)NC)ccc1NCc1ccc(C(F)(F)F)cc1]

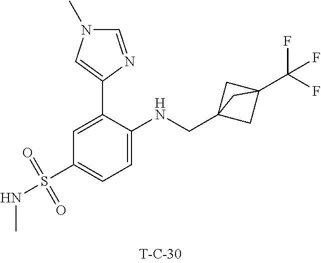 [SMILES: CNS(=O)(=O)c1ccc(NCC23CC(C(F)(F)F)(C2)C3)c(-c2cn(C)cn2)c1]

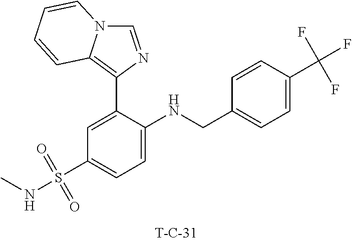 [SMILES: CNS(=O)(=O)c1ccc(NCc2ccc(C(F)(F)F)cc2)c(-c2ncn3ccccc23)c1]

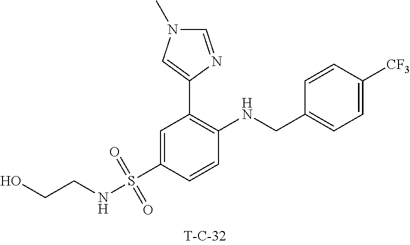 [SMILES: Cn1cnc(-c2cc(S(=O)(=O)NCCO)ccc2NCc2ccc(C(F)(F)F)cc2)c1]

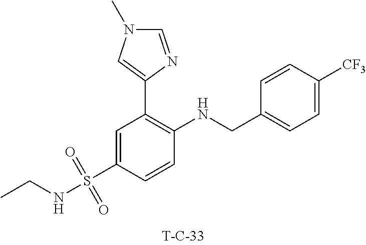 [SMILES: CCNS(=O)(=O)c1ccc(NCc2ccc(C(F)(F)F)cc2)c(-c2cn(C)cn2)c1]

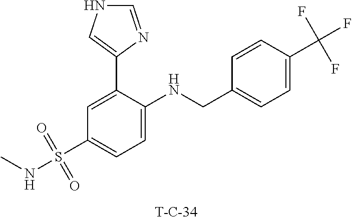 [SMILES: CNS(=O)(=O)c1ccc(NCc2ccc(C(F)(F)F)cc2)c(-c2c[nH]cn2)c1]

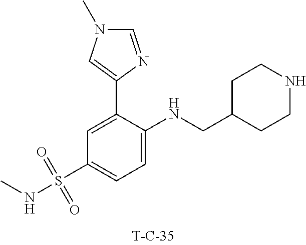 [SMILES: CNS(=O)(=O)c1ccc(NCC2CCNCC2)c(-c2cn(C)cn2)c1]

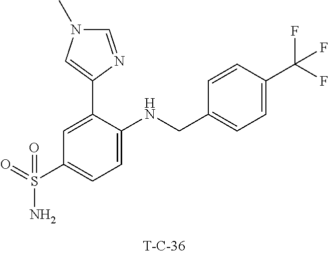 [SMILES: Cn1cnc(-c2cc(S(N)(=O)=O)ccc2NCc2ccc(C(F)(F)F)cc2)c1]

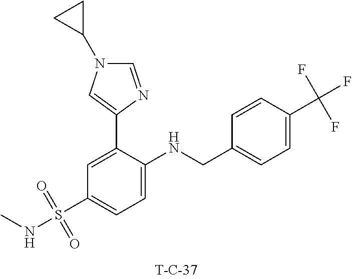 [SMILES: CNS(=O)(=O)c1ccc(NCc2ccc(C(F)(F)F)cc2)c(-c2cn(C3CC3)cn2)c1]

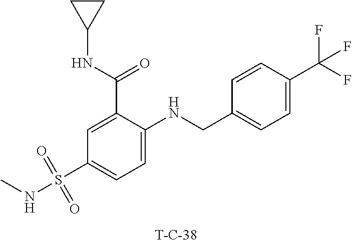 [SMILES: CNS(=O)(=O)c1ccc(NCc2ccc(C(F)(F)F)cc2)c(C(=O)NC2CC2)c1]

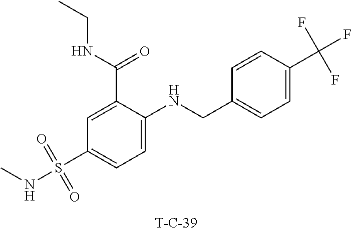 [SMILES: CCNC(=O)c1cc(S(=O)(=O)NC)ccc1NCc1ccc(C(F)(F)F)cc1]

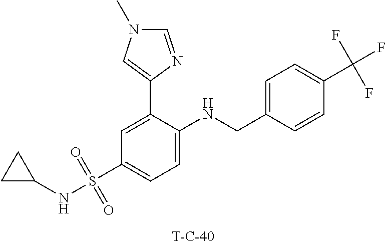 [SMILES: Cn1cnc(-c2cc(S(=O)(=O)NC3CC3)ccc2NCc2ccc(C(F)(F)F)cc2)c1]

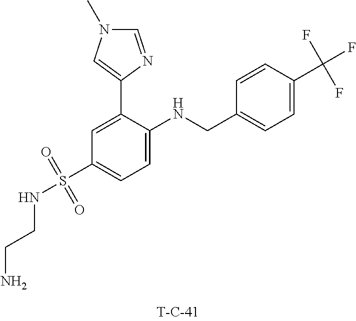 [SMILES: Cn1cnc(-c2cc(S(=O)(=O)NCCN)ccc2NCc2ccc(C(F)(F)F)cc2)c1]

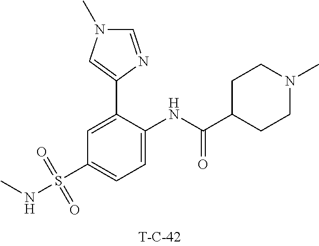

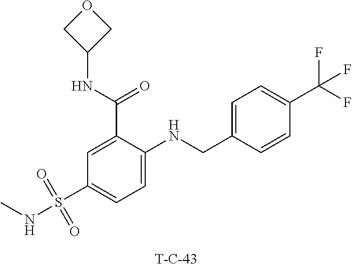 [SMILES: CNS(=O)(=O)c1ccc(NCc2ccc(C(F)(F)F)cc2)c(C(=O)NC2COC2)c1]

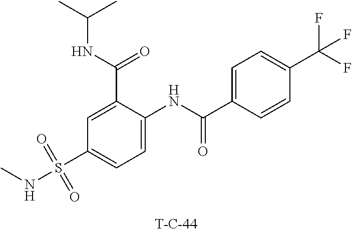 [SMILES: CNS(=O)(=O)c1ccc(NC(=O)c2ccc(C(F)(F)F)cc2)c(C(=O)NC(C)C)c1]

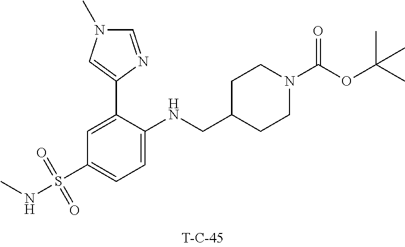 [SMILES: CNS(=O)(=O)c1ccc(NCC2CCN(C(=O)OC(C)(C)C)CC2)c(-c2cn(C)cn2)c1]

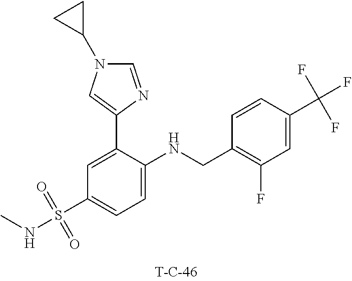 [SMILES: CNS(=O)(=O)c1ccc(NCc2ccc(C(F)(F)F)cc2F)c(-c2cn(C3CC3)cn2)c1]

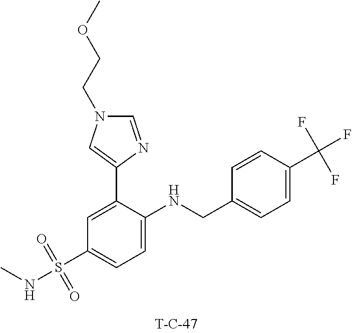 [SMILES: CNS(=O)(=O)c1ccc(NCc2ccc(C(F)(F)F)cc2)c(-c2cn(CCOC)cn2)c1]

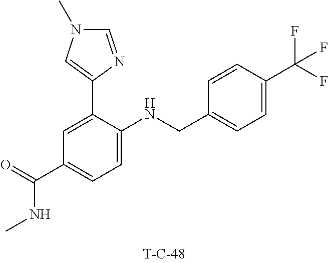 [SMILES: CNC(=O)c1ccc(NCc2ccc(C(F)(F)F)cc2)c(-c2cn(C)cn2)c1]

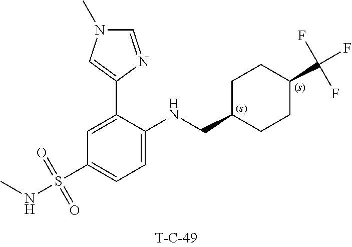 [SMILES: CNS(=O)(=O)c1ccc(NC[C@H]2CC[C@@H](C(F)(F)F)CC2)c(-c2cn(C)cn2)c1]

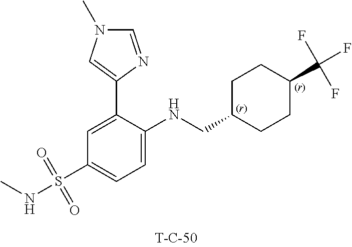 [SMILES: CNS(=O)(=O)c1ccc(NC[C@H]2CC[C@H](C(F)(F)F)CC2)c(-c2cn(C)cn2)c1]

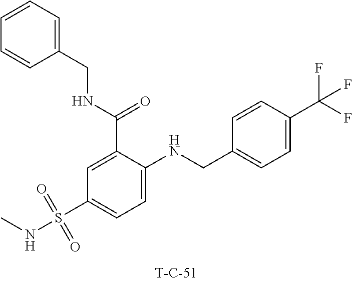 [SMILES: CNS(=O)(=O)c1ccc(NCc2ccc(C(F)(F)F)cc2)c(C(=O)NCc2ccccc2)c1]

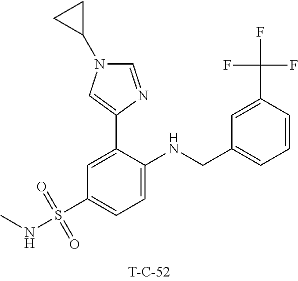 [SMILES: CNS(=O)(=O)c1ccc(NCc2cccc(C(F)(F)F)c2)c(-c2cn(C3CC3)cn2)c1]

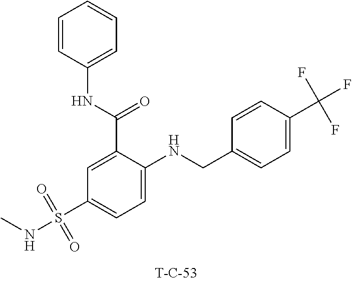 [SMILES: CNS(=O)(=O)c1ccc(NCc2ccc(C(F)(F)F)cc2)c(C(=O)Nc2ccccc2)c1]

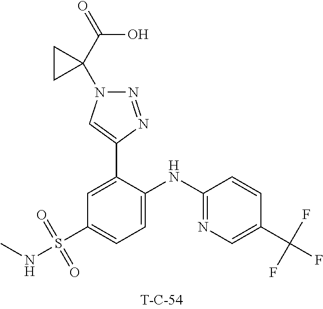 [SMILES: CNS(=O)(=O)c1ccc(Nc2ccc(C(F)(F)F)cn2)c(-c2cn(C3(C(=O)O)CC3)nn2)c1]

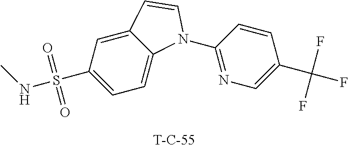 [SMILES: CNS(=O)(=O)c1ccc2c(ccn2-c2ccc(C(F)(F)F)cn2)c1]

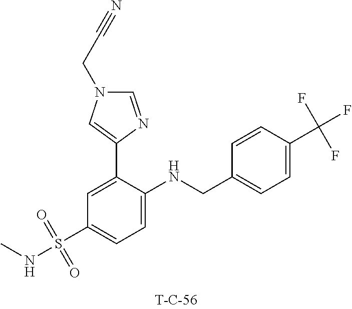 [SMILES: CNS(=O)(=O)c1ccc(NCc2ccc(C(F)(F)F)cc2)c(-c2cn(CC#N)cn2)c1]

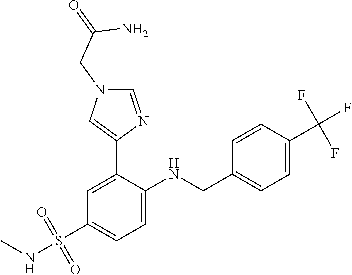 [SMILES: CNS(=O)(=O)c1ccc(NCc2ccc(C(F)(F)F)cc2)c(-c2cn(CC(N)=O)cn2)c1]

T-C-57

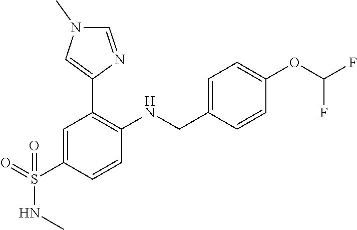 [SMILES: CNS(=O)(=O)c1ccc(NCc2ccc(OC(F)F)cc2)c(-c2cn(C)cn2)c1]

T-C-58

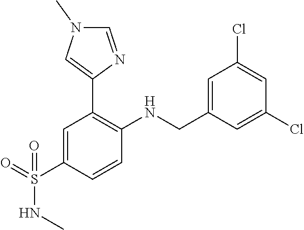 [SMILES: CNS(=O)(=O)c1ccc(NCc2cc(Cl)cc(Cl)c2)c(-c2cn(C)cn2)c1]

T-C-59

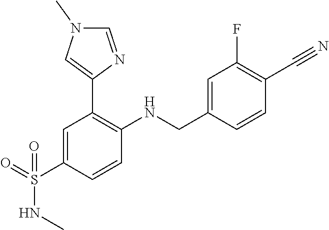 [SMILES: CNS(=O)(=O)c1ccc(NCc2ccc(C#N)c(F)c2)c(-c2cn(C)cn2)c1]

T-C-60

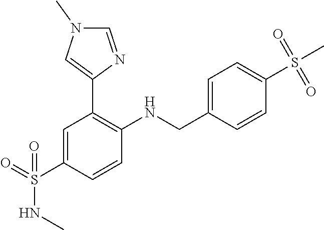 [SMILES: CNS(=O)(=O)c1ccc(NCc2ccc(S(C)(=O)=O)cc2)c(-c2cn(C)cn2)c1]

T-C-61

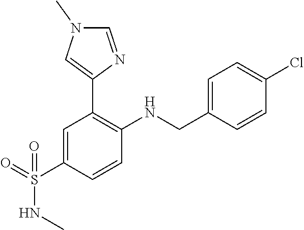 [SMILES: CNS(=O)(=O)c1ccc(NCc2ccc(Cl)cc2)c(-c2cn(C)cn2)c1]

T-C-62

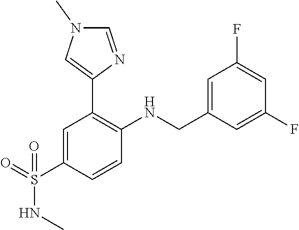 [SMILES: CNS(=O)(=O)c1ccc(NCc2cc(F)cc(F)c2)c(-c2cn(C)cn2)c1]

T-C-63

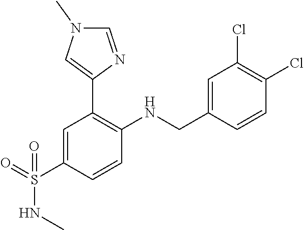 [SMILES: CNS(=O)(=O)c1ccc(NCc2ccc(Cl)c(Cl)c2)c(-c2cn(C)cn2)c1]

T-C-64

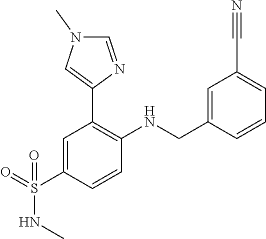 [SMILES: CNS(=O)(=O)c1ccc(NCc2cccc(C#N)c2)c(-c2cn(C)cn2)c1]

T-C-65

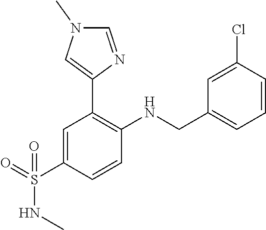 [SMILES: CNS(=O)(=O)c1ccc(NCc2cccc(Cl)c2)c(-c2cn(C)cn2)c1]

T-C-66

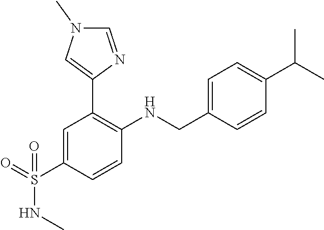 [SMILES: CNS(=O)(=O)c1ccc(NCc2ccc(C(C)C)cc2)c(-c2cn(C)cn2)c1]

T-C-67

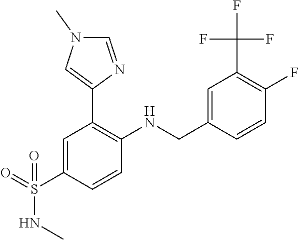 [SMILES: CNS(=O)(=O)c1ccc(NCc2ccc(F)c(C(F)(F)F)c2)c(-c2cn(C)cn2)c1]

T-C-68

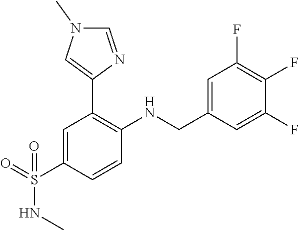 [SMILES: CNS(=O)(=O)c1ccc(NCc2cc(F)c(F)c(F)c2)c(-c2cn(C)cn2)c1]

T-C-69

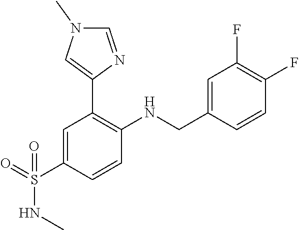 [SMILES: CNS(=O)(=O)c1ccc(NCc2ccc(F)c(F)c2)c(-c2cn(C)cn2)c1]

T-C-70

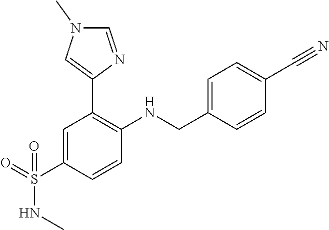 [SMILES: CNS(=O)(=O)c1ccc(NCc2ccc(C#N)cc2)c(-c2cn(C)cn2)c1]

T-C-71

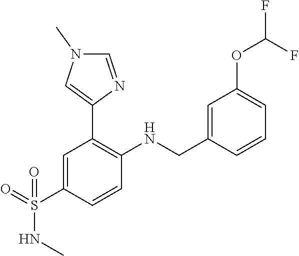 [SMILES: CNS(=O)(=O)c1ccc(NCc2cccc(OC(F)F)c2)c(-c2cn(C)cn2)c1]

T-C-72

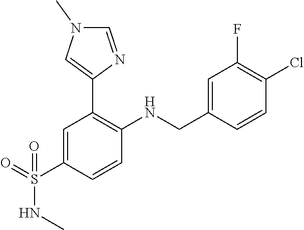 [SMILES: CNS(=O)(=O)c1ccc(NCc2ccc(Cl)c(F)c2)c(-c2cn(C)cn2)c1]

T-C-73

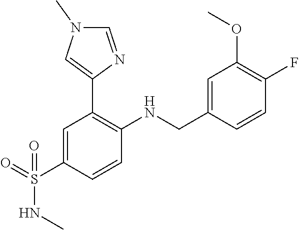 [SMILES: CNS(=O)(=O)c1ccc(NCc2ccc(F)c(OC)c2)c(-c2cn(C)cn2)c1]

T-C-74

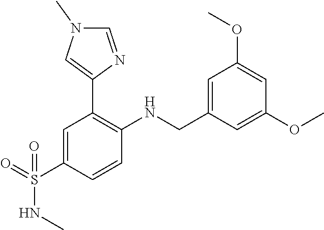 [SMILES: CNS(=O)(=O)c1ccc(NCc2cc(OC)cc(OC)c2)c(-c2cn(C)cn2)c1]

T-C-75

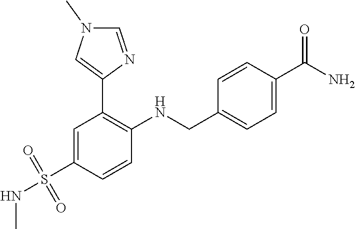 [SMILES: CNS(=O)(=O)c1ccc(NCc2ccc(C(N)=O)cc2)c(-c2cn(C)cn2)c1]

T-C-76

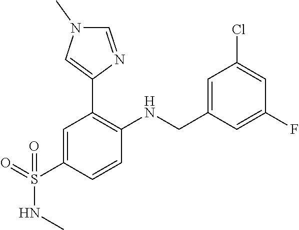 [SMILES: CNS(=O)(=O)c1ccc(NCc2cc(F)cc(Cl)c2)c(-c2cn(C)cn2)c1]

T-C-77

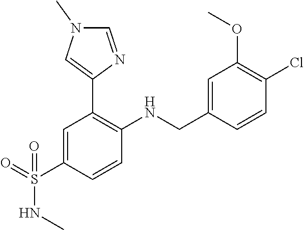 [SMILES: CNS(=O)(=O)c1ccc(NCc2ccc(Cl)c(OC)c2)c(-c2cn(C)cn2)c1]

T-C-78

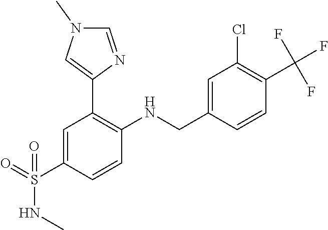 [SMILES: CNS(=O)(=O)c1ccc(NCc2ccc(C(F)(F)F)c(Cl)c2)c(-c2cn(C)cn2)c1]

T-C-79

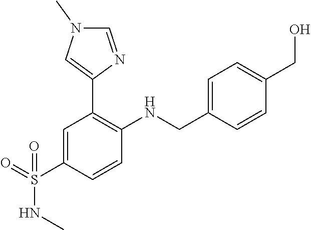 [SMILES: CNS(=O)(=O)c1ccc(NCc2ccc(CO)cc2)c(-c2cn(C)cn2)c1]

T-C-80

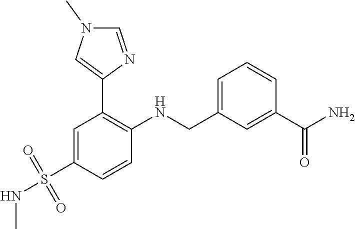 [SMILES: CNS(=O)(=O)c1ccc(NCc2cccc(C(N)=O)c2)c(-c2cn(C)cn2)c1]

T-C-81

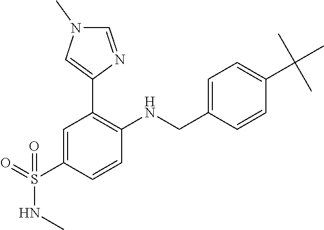 [SMILES: CNS(=O)(=O)c1ccc(NCc2ccc(C(C)(C)C)cc2)c(-c2cn(C)cn2)c1]

T-C-82

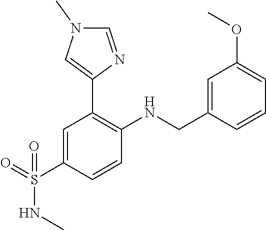 [SMILES: CNS(=O)(=O)c1ccc(NCc2cccc(OC)c2)c(-c2cn(C)cn2)c1]

T-C-83

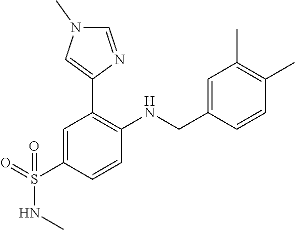 [SMILES: CNS(=O)(=O)c1ccc(NCc2ccc(C)c(C)c2)c(-c2cn(C)cn2)c1]

T-C-84

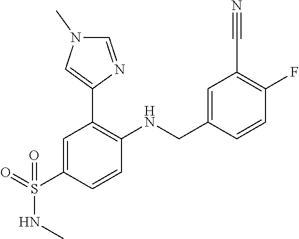 [SMILES: CNS(=O)(=O)c1ccc(NCc2ccc(F)c(C#N)c2)c(-c2cn(C)cn2)c1]

T-C-85

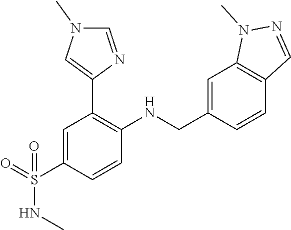 [SMILES: CNS(=O)(=O)c1ccc(NCc2ccc3cnn(C)c3c2)c(-c2cn(C)cn2)c1]

T-C-86

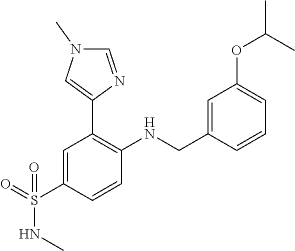 [SMILES: CNS(=O)(=O)c1ccc(NCc2cccc(OC(C)C)c2)c(-c2cn(C)cn2)c1]

T-C-87

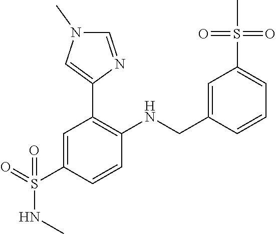 [SMILES: CNS(=O)(=O)c1ccc(NCc2cccc(S(C)(=O)=O)c2)c(-c2cn(C)cn2)c1]

T-C-88

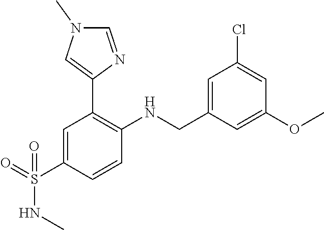 [SMILES: CNS(=O)(=O)c1ccc(NCc2cc(Cl)cc(OC)c2)c(-c2cn(C)cn2)c1]

T-C-89

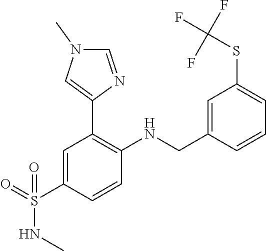 [SMILES: CNS(=O)(=O)c1ccc(NCc2cccc(SC(F)(F)F)c2)c(-c2cn(C)cn2)c1]

T-C-90

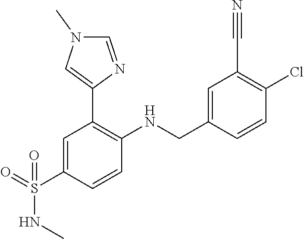 [SMILES: CNS(=O)(=O)c1ccc(NCc2ccc(Cl)c(C#N)c2)c(-c2cn(C)cn2)c1]

T-C-91

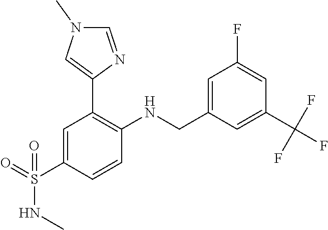 [SMILES: CNS(=O)(=O)c1ccc(NCc2cc(F)cc(C(F)(F)F)c2)c(-c2cn(C)cn2)c1]

T-C-92

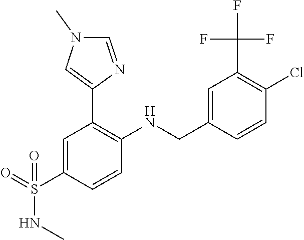 [SMILES: CNS(=O)(=O)c1ccc(NCc2ccc(Cl)c(C(F)(F)F)c2)c(-c2cn(C)cn2)c1]

T-C-93

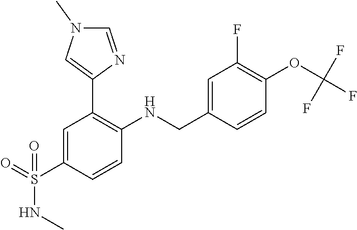 [SMILES: CNS(=O)(=O)c1ccc(NCc2ccc(OC(F)(F)F)c(F)c2)c(-c2cn(C)cn2)c1]

T-C-94

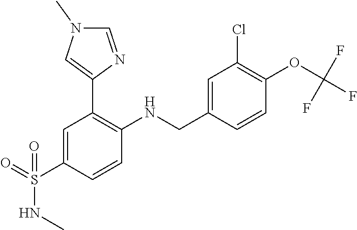 [SMILES: CNS(=O)(=O)c1ccc(NCc2ccc(OC(F)(F)F)c(Cl)c2)c(-c2cn(C)cn2)c1]

T-C-95

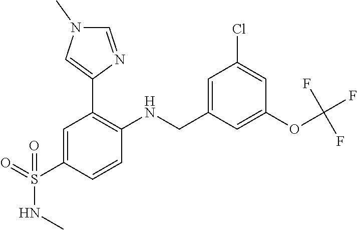 [SMILES: CNS(=O)(=O)c1ccc(NCc2cc(Cl)cc(OC(F)(F)F)c2)c(-c2cn(C)cn2)c1]

T-C-96

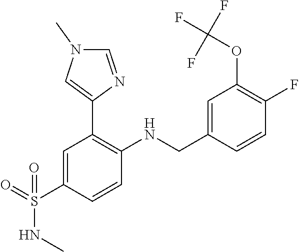 [SMILES: CNS(=O)(=O)c1ccc(NCc2ccc(F)c(OC(F)(F)F)c2)c(-c2cn(C)cn2)c1]

T-C-97

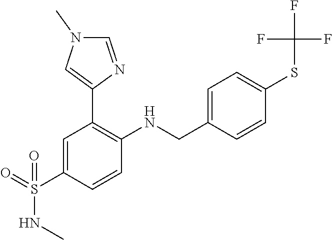 [SMILES: CNS(=O)(=O)c1ccc(NCc2ccc(SC(F)(F)F)cc2)c(-c2cn(C)cn2)c1]

T-C-98

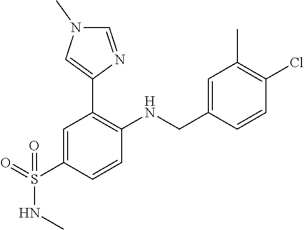 [SMILES: CNS(=O)(=O)c1ccc(NCc2ccc(Cl)c(C)c2)c(-c2cn(C)cn2)c1]

T-C-99

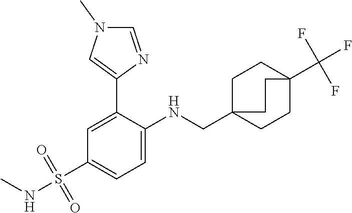 [SMILES: CNS(=O)(=O)c1ccc(NCC23CCC(C(F)(F)F)(CC2)CC3)c(-c2cn(C)cn2)c1]

T-C-100

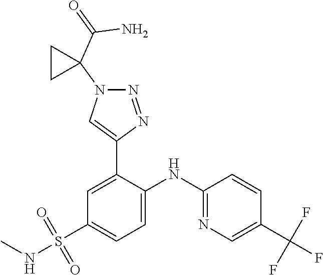 [SMILES: CNS(=O)(=O)c1ccc(Nc2ccc(C(F)(F)F)cn2)c(-c2cn(C3(C(N)=O)CC3)nn2)c1]

T-C-101

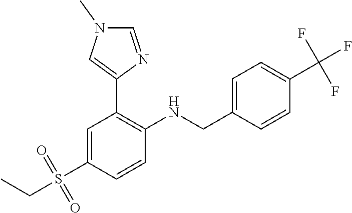 [SMILES: CCS(=O)(=O)c1ccc(NCc2ccc(C(F)(F)F)cc2)c(-c2cn(C)cn2)c1]

T-C-102

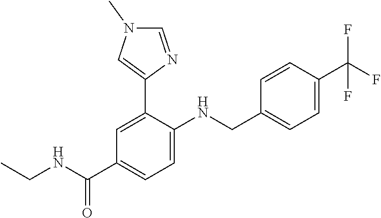 [SMILES: CCNC(=O)c1ccc(NCc2ccc(C(F)(F)F)cc2)c(-c2cn(C)cn2)c1]

T-C-103

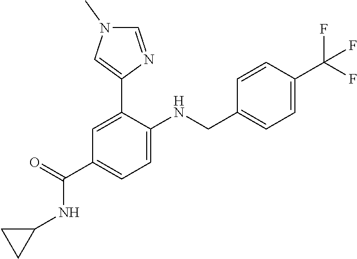 [SMILES: Cn1cnc(-c2cc(C(=O)NC3CC3)ccc2NCc2ccc(C(F)(F)F)cc2)c1]

T-C-104

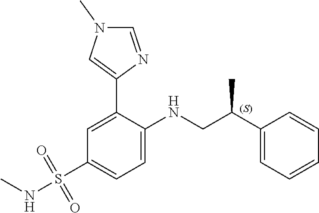

T-C-105

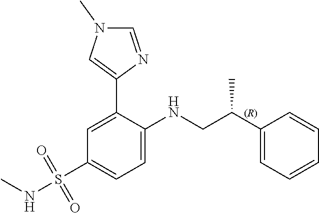

T-C-106

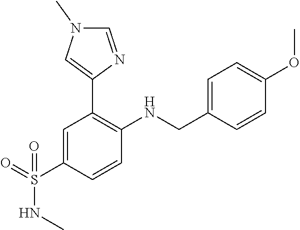 [SMILES: CNS(=O)(=O)c1ccc(NCc2ccc(OC)cc2)c(-c2cn(C)cn2)c1]

T-C-107

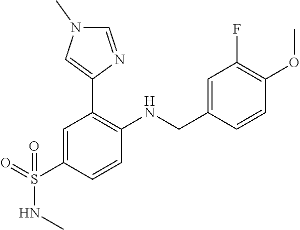 [SMILES: CNS(=O)(=O)c1ccc(NCc2ccc(OC)c(F)c2)c(-c2cn(C)cn2)c1]

T-C-108

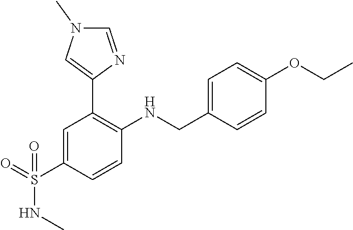 [SMILES: CCOc1ccc(CNc2ccc(S(=O)(=O)NC)cc2-c2cn(C)cn2)cc1]

T-C-109

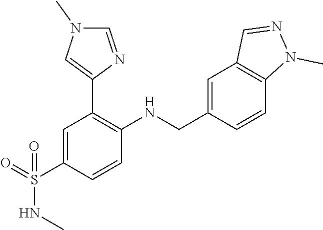 [SMILES: CNS(=O)(=O)c1ccc(NCc2ccc3c(cnn3C)c2)c(-c2cn(C)cn2)c1]

T-C-110

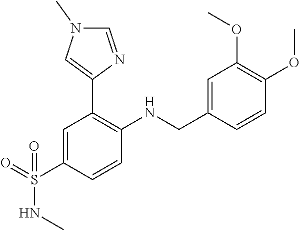 [SMILES: CNS(=O)(=O)c1ccc(NCc2ccc(OC)c(OC)c2)c(-c2cn(C)cn2)c1]

T-C-111

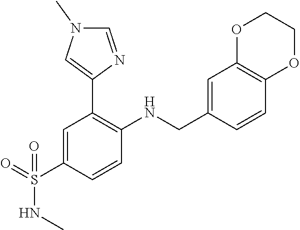 [SMILES: CNS(=O)(=O)c1ccc(NCc2ccc3c(c2)OCCO3)c(-c2cn(C)cn2)c1]

T-C-112

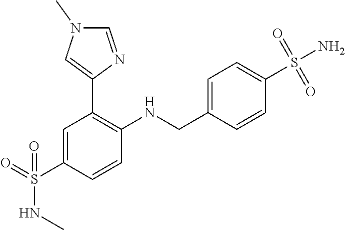 [SMILES: CNS(=O)(=O)c1ccc(NCc2ccc(S(N)(=O)=O)cc2)c(-c2cn(C)cn2)c1]

T-C-113

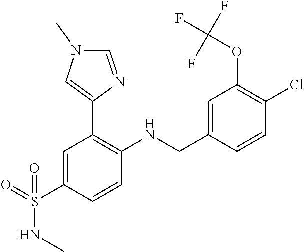 [SMILES: CNS(=O)(=O)c1ccc(NCc2ccc(Cl)c(OC(F)(F)F)c2)c(-c2cn(C)cn2)c1]

T-C-114

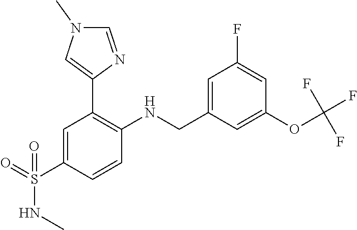 [SMILES: CNS(=O)(=O)c1ccc(NCc2cc(F)cc(OC(F)(F)F)c2)c(-c2cn(C)cn2)c1]

T-C-115

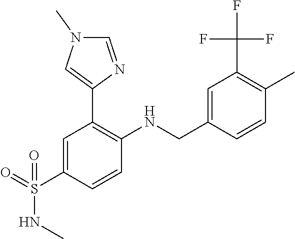 [SMILES: CNS(=O)(=O)c1ccc(NCc2ccc(C)c(C(F)(F)F)c2)c(-c2cn(C)cn2)c1]

T-C-116

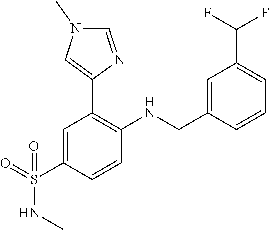 [SMILES: CNS(=O)(=O)c1ccc(NCc2cccc(C(F)F)c2)c(-c2cn(C)cn2)c1]

T-C-117

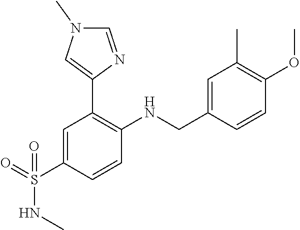 [SMILES: CNS(=O)(=O)c1ccc(NCc2ccc(OC)c(C)c2)c(-c2cn(C)cn2)c1]

T-C-118

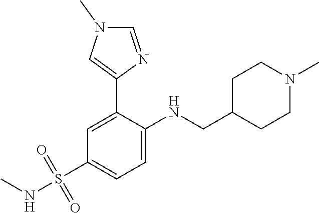

T-C-119

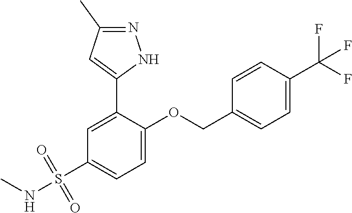 [SMILES: CNS(=O)(=O)c1ccc(OCc2ccc(C(F)(F)F)cc2)c(-c2cc(C)n[nH]2)c1]

T-C-120

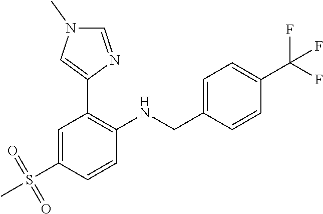 [SMILES: Cn1cnc(-c2cc(S(C)(=O)=O)ccc2NCc2ccc(C(F)(F)F)cc2)c1]

T-C-121

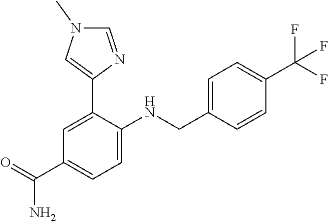 [SMILES: Cn1cnc(-c2cc(C(N)=O)ccc2NCc2ccc(C(F)(F)F)cc2)c1]

T-C-122

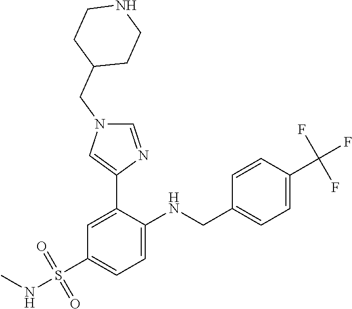 [SMILES: CNS(=O)(=O)c1ccc(NCc2ccc(C(F)(F)F)cc2)c(-c2cn(CC3CCNCC3)cn2)c1]

T-C-123

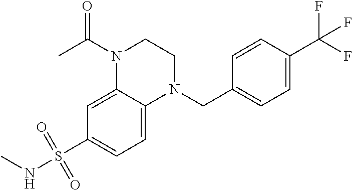 [SMILES: CNS(=O)(=O)c1ccc2c(c1)N(C(C)=O)CCN2Cc1ccc(C(F)(F)F)cc1]

T-C-124

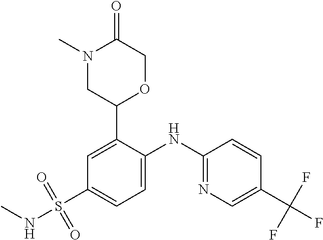 [SMILES: CNS(=O)(=O)c1ccc(Nc2ccc(C(F)(F)F)cn2)c(C2CN(C)C(=O)CO2)c1]

T-C-125

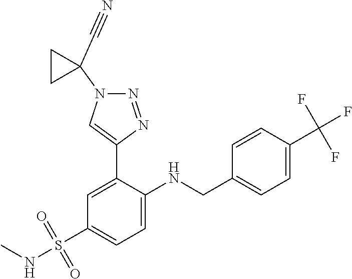 [SMILES: CNS(=O)(=O)c1ccc(NCc2ccc(C(F)(F)F)cc2)c(-c2cn(C3(C#N)CC3)nn2)c1]

T-C-126

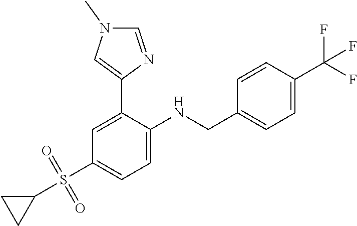 [SMILES: Cn1cnc(-c2cc(S(=O)(=O)C3CC3)ccc2NCc2ccc(C(F)(F)F)cc2)c1]

T-C-127

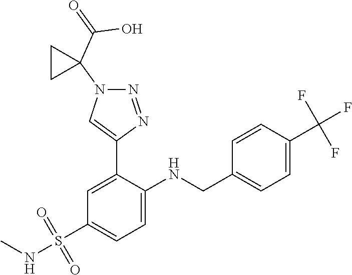 [SMILES: CNS(=O)(=O)c1ccc(NCc2ccc(C(F)(F)F)cc2)c(-c2cn(C3(C(=O)O)CC3)nn2)c1]

T-C-128

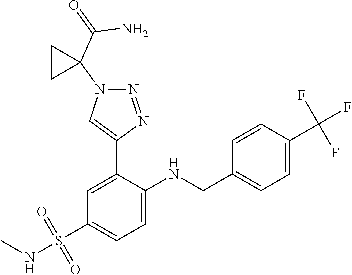 [SMILES: CNS(=O)(=O)c1ccc(NCc2ccc(C(F)(F)F)cc2)c(-c2cn(C3(C(N)=O)CC3)nn2)c1]

T-C-129

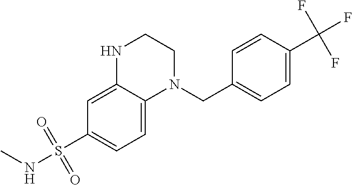 [SMILES: CNS(=O)(=O)c1ccc2c(c1)NCCN2Cc1ccc(C(F)(F)F)cc1]

T-C-130

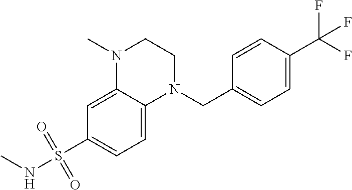 [SMILES: CNS(=O)(=O)c1ccc2c(c1)N(C)CCN2Cc1ccc(C(F)(F)F)cc1]

T-C-131

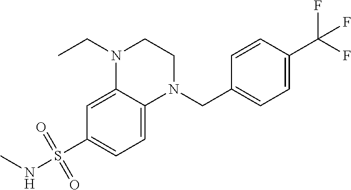 [SMILES: CCN1CCN(Cc2ccc(C(F)(F)F)cc2)c2ccc(S(=O)(=O)NC)cc21]

T-C-132

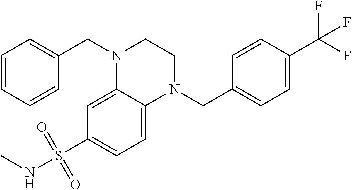 [SMILES: CNS(=O)(=O)c1ccc2c(c1)N(Cc1ccccc1)CCN2Cc1ccc(C(F)(F)F)cc1]

T-C-133

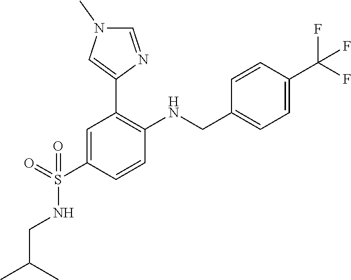 [SMILES: CC(C)CNS(=O)(=O)c1ccc(NCc2ccc(C(F)(F)F)cc2)c(-c2cn(C)cn2)c1]

T-C-134

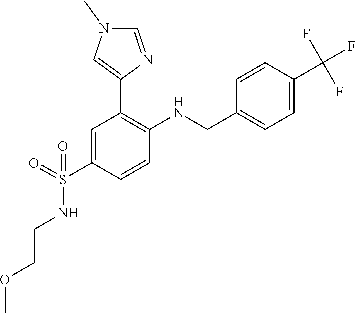 [SMILES: COCCNS(=O)(=O)c1ccc(NCc2ccc(C(F)(F)F)cc2)c(-c2cn(C)cn2)c1]

T-C-135

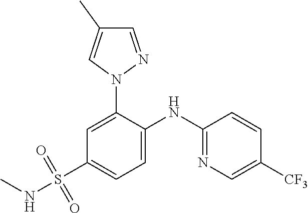 [SMILES: CNS(=O)(=O)c1ccc(Nc2ccc(C(F)(F)F)cn2)c(-n2cc(C)cn2)c1]

T-C-136

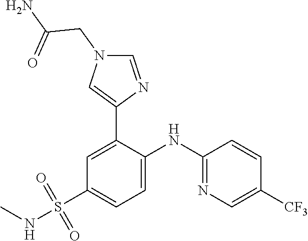 [SMILES: CNS(=O)(=O)c1ccc(Nc2ccc(C(F)(F)F)cn2)c(-c2cn(CC(N)=O)cn2)c1]

T-C-137

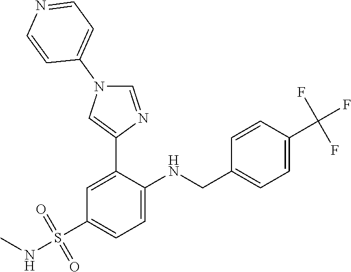 [SMILES: CNS(=O)(=O)c1ccc(NCc2ccc(C(F)(F)F)cc2)c(-c2cn(-c3ccncc3)cn2)c1]

T-C-138

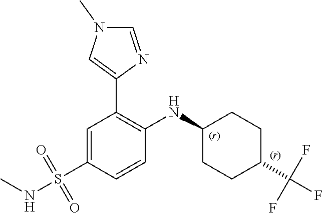 [SMILES: CNS(=O)(=O)c1ccc(N[C@H]2CC[C@H](C(F)(F)F)CC2)c(-c2cn(C)cn2)c1]

T-C-139

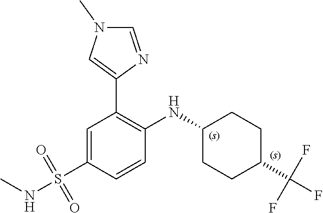 [SMILES: CNS(=O)(=O)c1ccc(N[C@H]2CC[C@@H](C(F)(F)F)CC2)c(-c2cn(C)cn2)c1]

T-C-140

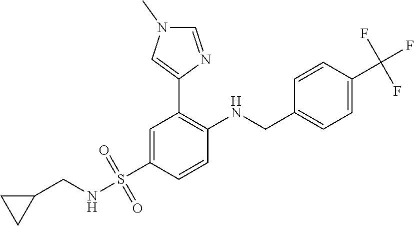 [SMILES: Cn1cnc(-c2cc(S(=O)(=O)NCC3CC3)ccc2NCc2ccc(C(F)(F)F)cc2)c1]

T-C-141

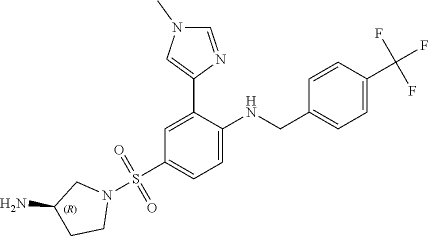

T-C-142

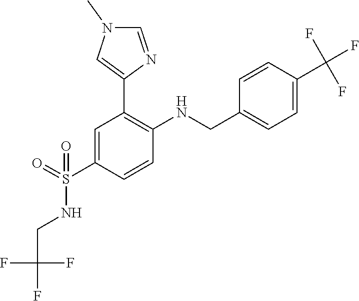 [SMILES: Cn1cnc(-c2cc(S(=O)(=O)NCC(F)(F)F)ccc2NCc2ccc(C(F)(F)F)cc2)c1]

T-C-143

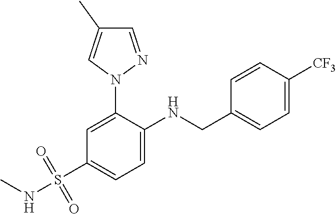 [SMILES: CNS(=O)(=O)c1ccc(NCc2ccc(C(F)(F)F)cc2)c(-n2cc(C)cn2)c1]

T-C-144

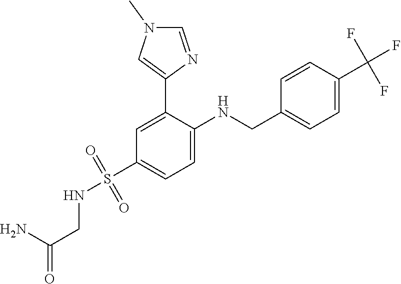 [SMILES: Cn1cnc(-c2cc(S(=O)(=O)NCC(N)=O)ccc2NCc2ccc(C(F)(F)F)cc2)c1]

T-C-145

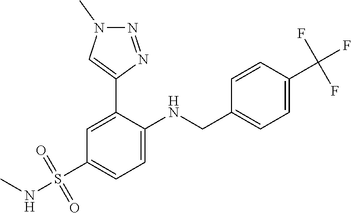 [SMILES: CNS(=O)(=O)c1ccc(NCc2ccc(C(F)(F)F)cc2)c(-c2cn(C)nn2)c1]

T-C-146

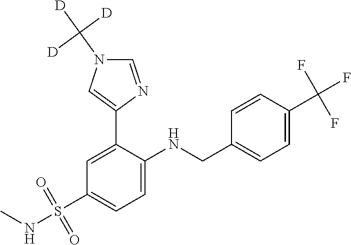 [SMILES: [2H]C([2H])([2H])n1cnc(-c2cc(S(=O)(=O)NC)ccc2NCc2ccc(C(F)(F)F)cc2)c1]

T-C-147

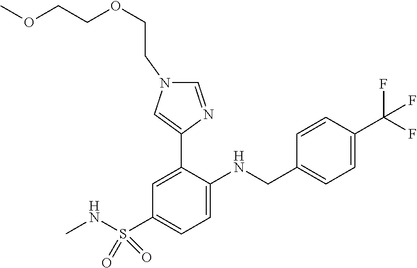 [SMILES: CNS(=O)(=O)c1ccc(NCc2ccc(C(F)(F)F)cc2)c(-c2cn(CCOCCOC)cn2)c1]

T-C-148

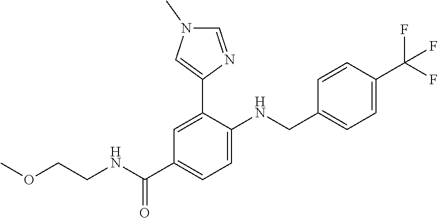 [SMILES: COCCNC(=O)c1ccc(NCc2ccc(C(F)(F)F)cc2)c(-c2cn(C)cn2)c1]

T-C-149

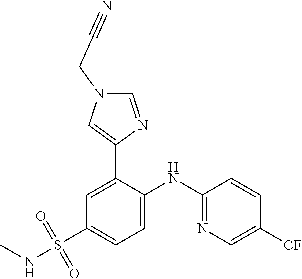

T-C-150

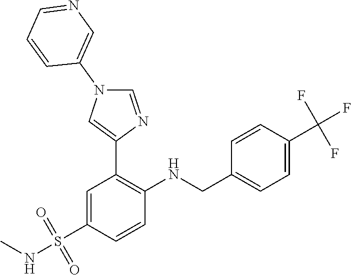 [SMILES: CNS(=O)(=O)c1ccc(NCc2ccc(C(F)(F)F)cc2)c(-c2cn(-c3cccnc3)cn2)c1]

T-C-151

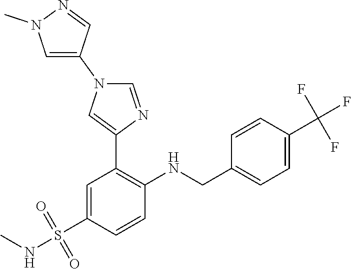 [SMILES: CNS(=O)(=O)c1ccc(NCc2ccc(C(F)(F)F)cc2)c(-c2cn(-c3cnn(C)c3)cn2)c1]

T-C-152

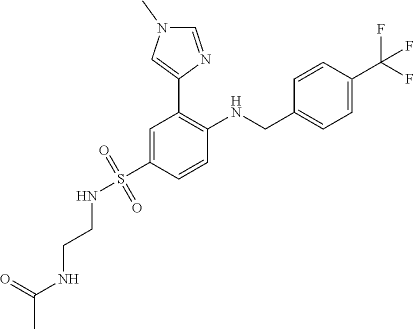 [SMILES: CC(=O)NCCNS(=O)(=O)c1ccc(NCc2ccc(C(F)(F)F)cc2)c(-c2cn(C)cn2)c1]

T-C-153

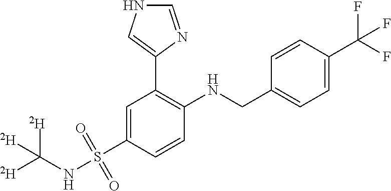 [SMILES: [2H]C([2H])([2H])NS(=O)(=O)c1ccc(NCc2ccc(C(F)(F)F)cc2)c(-c2c[nH]cn2)c1]

T-C-154

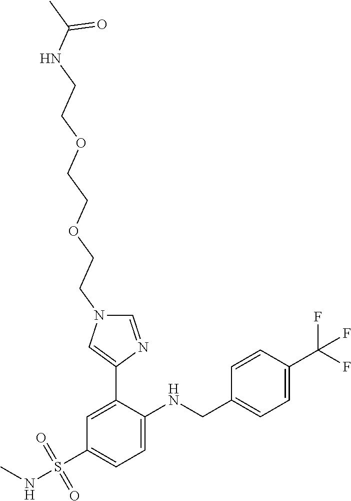 [SMILES: CNS(=O)(=O)c1ccc(NCc2ccc(C(F)(F)F)cc2)c(-c2cn(CCOCCOCCNC(C)=O)cn2)c1]

T-C-155

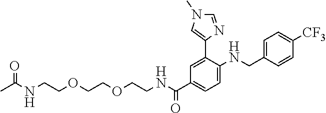 [SMILES: CC(=O)NCCOCCOCCNC(=O)c1ccc(NCc2ccc(C(F)(F)F)cc2)c(-c2cn(C)cn2)c1]

T-C-156

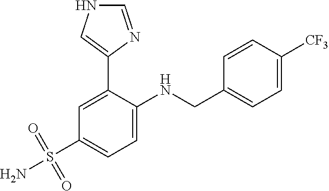 [SMILES: NS(=O)(=O)c1ccc(NCc2ccc(C(F)(F)F)cc2)c(-c2c[nH]cn2)c1]

T-C-157

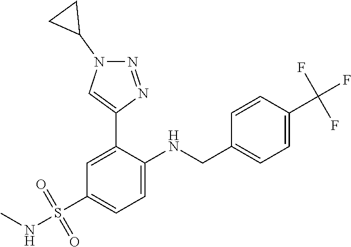 [SMILES: CNS(=O)(=O)c1ccc(NCc2ccc(C(F)(F)F)cc2)c(-c2cn(C3CC3)nn2)c1]

T-C-158

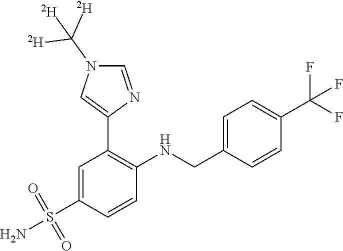 [SMILES: [2H]C([2H])([2H])n1cnc(-c2cc(S(N)(=O)=O)ccc2NCc2ccc(C(F)(F)F)cc2)c1]

T-C-159

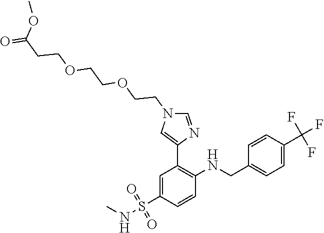 [SMILES: CNS(=O)(=O)c1ccc(NCc2ccc(C(F)(F)F)cc2)c(-c2cn(CCOCCOCCC(=O)OC)cn2)c1]

T-C-160

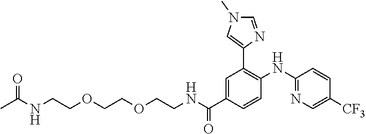 [SMILES: CC(=O)NCCOCCOCCNC(=O)c1ccc(Nc2ccc(C(F)(F)F)cn2)c(-c2cn(C)cn2)c1]

T-C-161

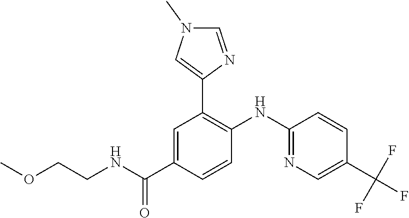 [SMILES: COCCNC(=O)c1ccc(Nc2ccc(C(F)(F)F)cn2)c(-c2cn(C)cn2)c1]

T-C-162

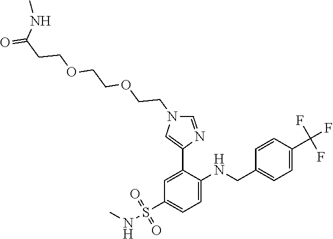 [SMILES: CNC(=O)CCOCCOCCn1cnc(-c2cc(S(=O)(=O)NC)ccc2NCc2ccc(C(F)(F)F)cc2)c1]

T-C-163

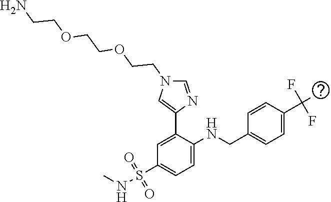 [SMILES: CNS(=O)(=O)c1ccc(NCc2ccc(C(C)(F)F)cc2)c(-c2cn(CCOCCOCCN)cn2)c1]

T-C-164

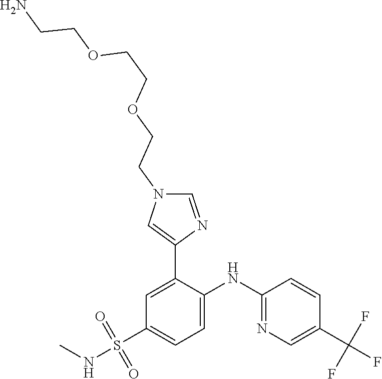 [SMILES: CNS(=O)(=O)c1ccc(Nc2ccc(C(F)(F)F)cn2)c(-c2cn(CCOCCOCCN)cn2)c1]

T-C-165

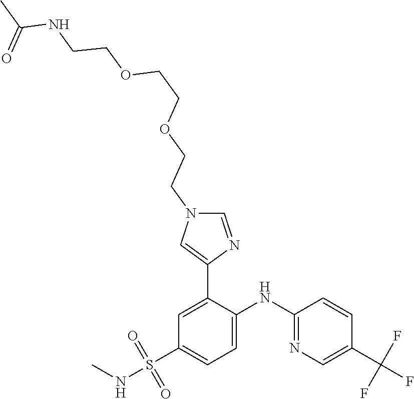 [SMILES: CNS(=O)(=O)c1ccc(Nc2ccc(C(F)(F)F)cn2)c(-c2cn(CCOCCOCCNC(C)=O)cn2)c1]

T-C-166

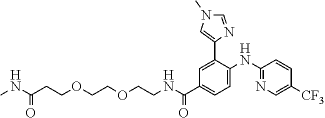 [SMILES: CNC(=O)CCOCCOCCNC(=O)c1ccc(Nc2ccc(C(F)(F)F)cn2)c(-c2cn(C)cn2)c1]

T-C-167

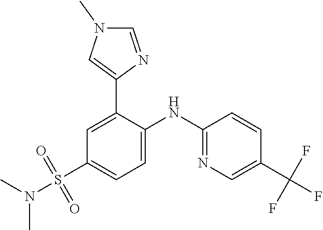 [SMILES: CN(C)S(=O)(=O)c1ccc(Nc2ccc(C(F)(F)F)cn2)c(-c2cn(C)cn2)c1]

T-C-168

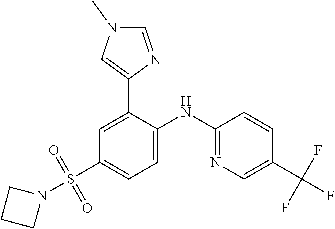 [SMILES: Cn1cnc(-c2cc(S(=O)(=O)N3CCC3)ccc2Nc2ccc(C(F)(F)F)cn2)c1]

T-C-169

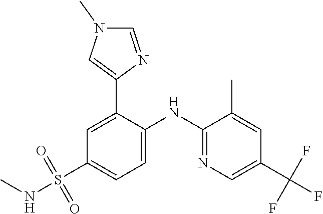 [SMILES: CNS(=O)(=O)c1ccc(Nc2ncc(C(F)(F)F)cc2C)c(-c2cn(C)cn2)c1]

T-C-170

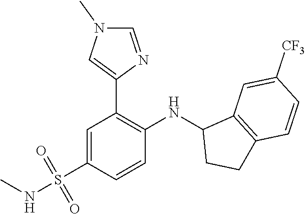 [SMILES: CNS(=O)(=O)c1ccc(NC2CCc3ccc(C(F)(F)F)cc32)c(-c2cn(C)cn2)c1]

T-C-171

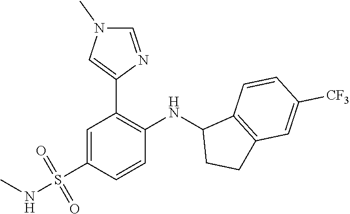 [SMILES: CNS(=O)(=O)c1ccc(NC2CCc3cc(C(F)(F)F)ccc32)c(-c2cn(C)cn2)c1]

T-C-172

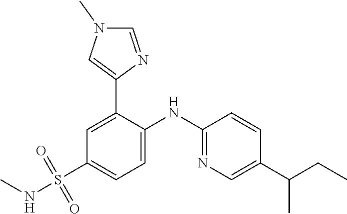 [SMILES: CCC(C)c1ccc(Nc2ccc(S(=O)(=O)NC)cc2-c2cn(C)cn2)nc1]

T-C-173

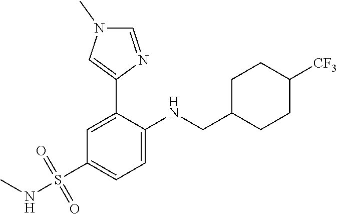 [SMILES: CNS(=O)(=O)c1ccc(NCC2CCC(C(F)(F)F)CC2)c(-c2cn(C)cn2)c1]

T-C-174

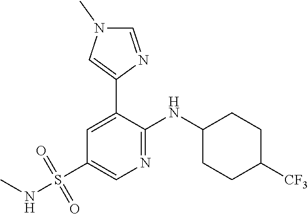 [SMILES: CNS(=O)(=O)c1cnc(NC2CCC(C(F)(F)F)CC2)c(-c2cn(C)cn2)c1]

T-C-175

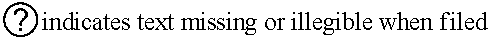

### 4. TEAD Inhibitors of Formulae D, and D-1 to D-85

In certain embodiments, a TEAD inhibitor is a compound of Formula D:

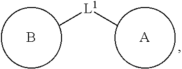

or a pharmaceutically acceptable salt thereof, wherein
L¹ is a covalent bound, or a C₁₋₆ bivalent straight or branched hydrocarbon chain wherein 1, 2, or 3 methylene units of the chain are independently and optionally replaced with —N(R)—, —O—, or —C(O)—;
Ring A is selected from

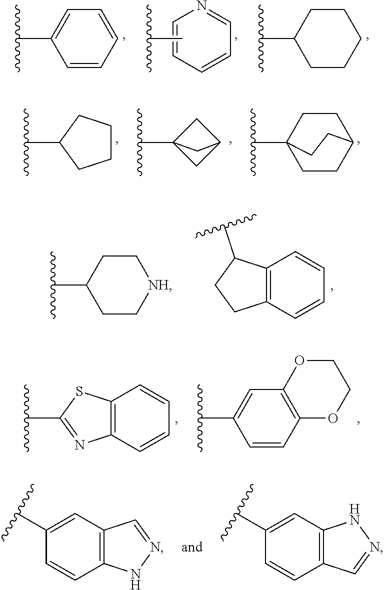

each of which is optionally substituted;
Ring B is

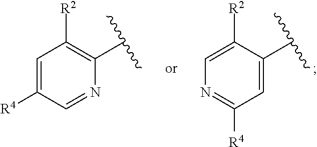

each R² is independently selected from —OR, —C(O)NR₂, optionally substituted —C₁₋₆ aliphatic,

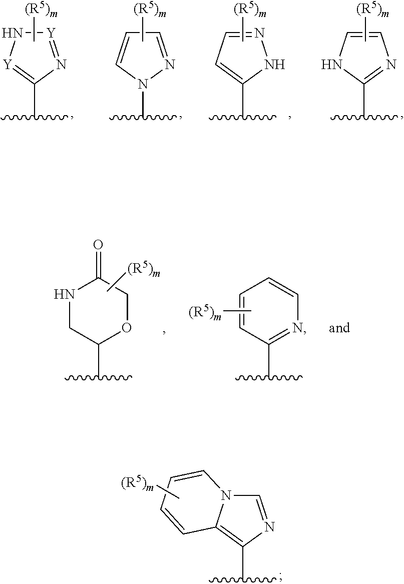

each Y is independently N or CR⁵;
each R⁴ is independently —S(O)₂NR₂, —S(O)₂R, —C(O)NR₂, —C(O)R, or optionally substituted —C₁₋₆ aliphatic;
each R⁵ is independently R, —CN, —C(O)R, —C(O)NR₂, or optionally substituted 5-6 membered heteroaryl having 1-2 heteroatoms independently selected from nitrogen, oxygen, or sulfur;
each m is independently 0, 1, or 2; and
each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

As defined generally above, L¹ is a covalent bound, or a C₁₋₆ bivalent straight or branched hydrocarbon chain wherein 1, 2, or 3 methylene units of the chain are independently and optionally replaced with —N(R)—, —O—, or —C(O)—.

In some embodiments, L¹ is a covalent bond.

In some embodiments, L¹ is C₁₋₆ bivalent straight or branched hydrocarbon chain wherein 1, 2, or 3 methylene units of the chain are independently and optionally replaced with —N(R)—.

In some embodiments, L¹ is C₁₋₆ bivalent straight or branched hydrocarbon chain wherein 1, 2, or 3 methylene units of the chain are independently and optionally replaced with —O—.

In some embodiments, L¹ is C₁₋₆ bivalent straight or branched hydrocarbon chain wherein 1, 2, or 3 methylene units of the chain are independently and optionally replaced with —C(O)—.

In some embodiments, L¹ is —NH—. In some embodiments, L¹ is —NH—CH₂—. In some embodiments, L¹ is —NH—CH₂—CH₂—. In some embodiments, L¹ is —CH₂—. In some embodiments, L¹ is

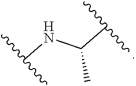 [SMILES: *N[C@H](*)C]

In some embodiments, L¹ is

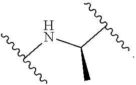 [SMILES: *N[C@@H](*)C]

In some embodiments, L¹ is

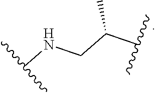 [SMILES: *NC[C@@H](*)C]

In some embodiments, L¹ is

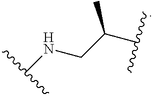 [SMILES: *NC[C@H](*)C]

In some embodiments, L¹ is —CH═CH—. In some embodiments, L¹ is

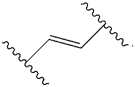 [SMILES: */C=C/*]

In some embodiments, L¹ is

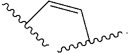 [SMILES: */C=C\*]

In some embodiments, L¹ is —NH—C(O)—.

In some embodiments, L¹ is selected from those depicted in Table D, below.

As defined generally above, Ring A is selected from

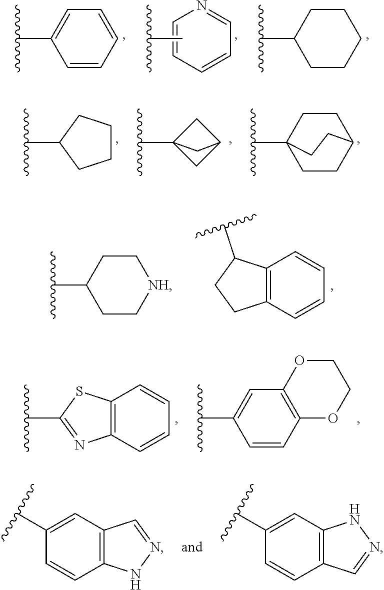

each of which is optionally substituted.

In some embodiments, Ring A is optionally substituted

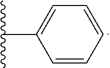 [SMILES: *c1ccccc1]

In some embodiments, Ring A is optionally substituted

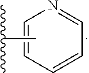

In some embodiments, Ring A is optionally substituted

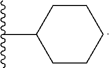 [SMILES: *C1CCCCC1]

In some embodiments, Ring A is optionally substituted

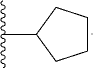 [SMILES: *C1CCCC1]

In some embodiments, Ring A is optionally substituted

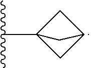 [SMILES: *C12CC(C1)C2]

In some embodiments, Ring A is optionally substituted

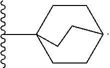 [SMILES: *C12CCC(CC1)CC2]

In some embodiments, Ring A is optionally substituted

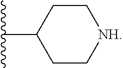 [SMILES: *C1CCNCC1]

In some embodiments, Ring A is optionally substituted

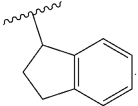 [SMILES: *C1CCc2ccccc21]

In some embodiments, Ring A is optionally substituted

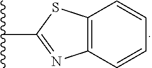 [SMILES: *c1nc2ccccc2s1]

In some embodiments, Ring A is optionally substituted

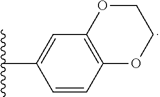 [SMILES: *c1ccc2c(c1)OCCO2]

In some embodiments, Ring A is optionally substituted

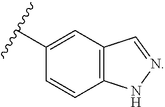 [SMILES: *c1ccc2[nH]ncc2c1]

In some embodiments, Ring A is optionally substituted

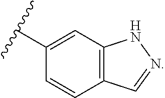 [SMILES: *c1ccc2cn[nH]c2c1]

In some embodiments, Ring A is selected from

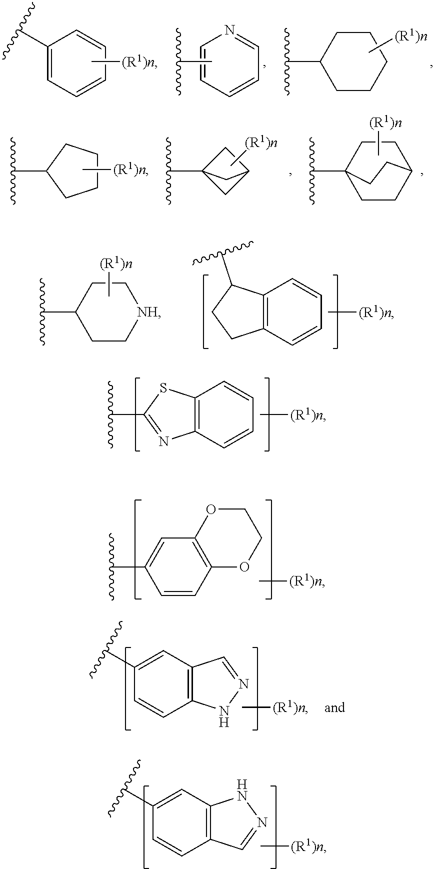

wherein each R¹ is independently R, halogen, —CN, —C(O)R, —C(O)NR₂, —OR, —SR, —S(O)₂NR₂, or —S(O)₂R, and each n is independently 0, 1, 2, or 3, wherein each R is independently as defined herein and as described in embodiments herein.

In some embodiments, R¹ is R. In some embodiments, R¹ is halogen. In some embodiments, R¹ is —CN. In some embodiments, R¹ is —C(O)R. In some embodiments, R¹ is —C(O)NR₂. In some embodiments, R¹ is —OR. In some embodiments, R¹ is —SR. In some embodiments, R¹ is —S(O)₂NR₂. In some embodiments, R¹ is —S(O)₂R.

In some embodiments, each R¹ is independently H, halogen, —C₁₋₆ aliphatic optionally substituted by 1-6 halogen, 3-8 membered saturated or partially unsaturated monocyclic carbocyclyl optionally substituted by 1-6 halogen, or 3-8 membered saturated or partially unsaturated monocyclic heterocyclyl having 1-2 heteroatoms independently selected from nitrogen, oxygen, or sulfur optionally substituted by 1-6 halogen.

In some embodiments, each R¹ is independently H, —CF₃, —C(O)NH₂, —CH₃, —CH₂CH₃, —OCH₃, —CHF₂, —OCF₃, —OCHF₂, —SCF₃, —Cl, —S(O)₂—NH₂, —OCH₂CH₃, —F, —C(O)NHCH₃, —CN, —S(O)₂—CH₃, —OCH(CH₃)₂, —CH(CH₃)₂, —C(CH₃)₃, —CH₂OH,

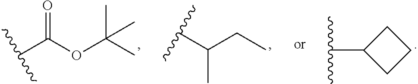

In some embodiments, each R¹ is independently selected from those depicted in Table D, below.

In some embodiments, n is 0. In some embodiments, n is 1. In some embodiments, n is 2. In some embodiments, n is 3.

In some embodiments, Ring A is selected from

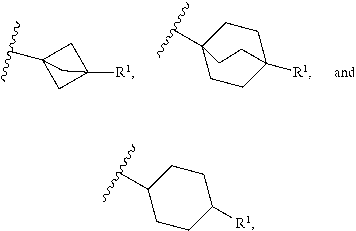

wherein each of R¹ is as defined herein and described in embodiments herein, both singly and in combination.

In some embodiments, Ring A is selected from

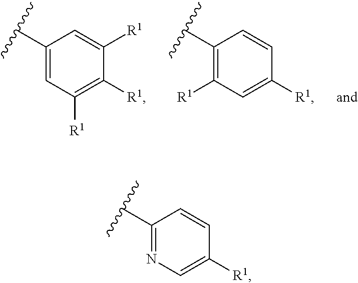

wherein each R¹ is as defined herein and described in embodiments herein, both singly and in combination.

In some embodiments, Ring A is

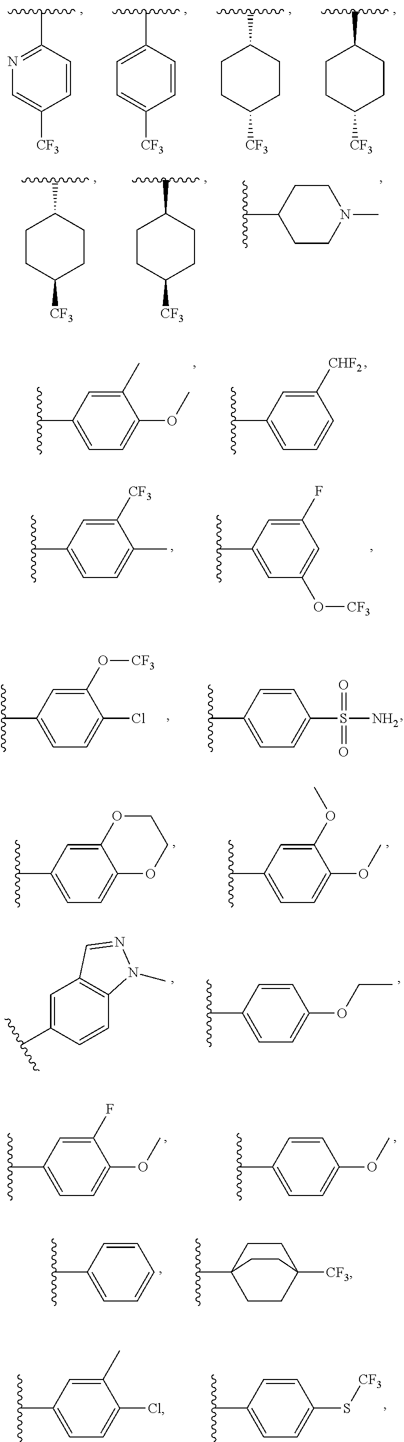

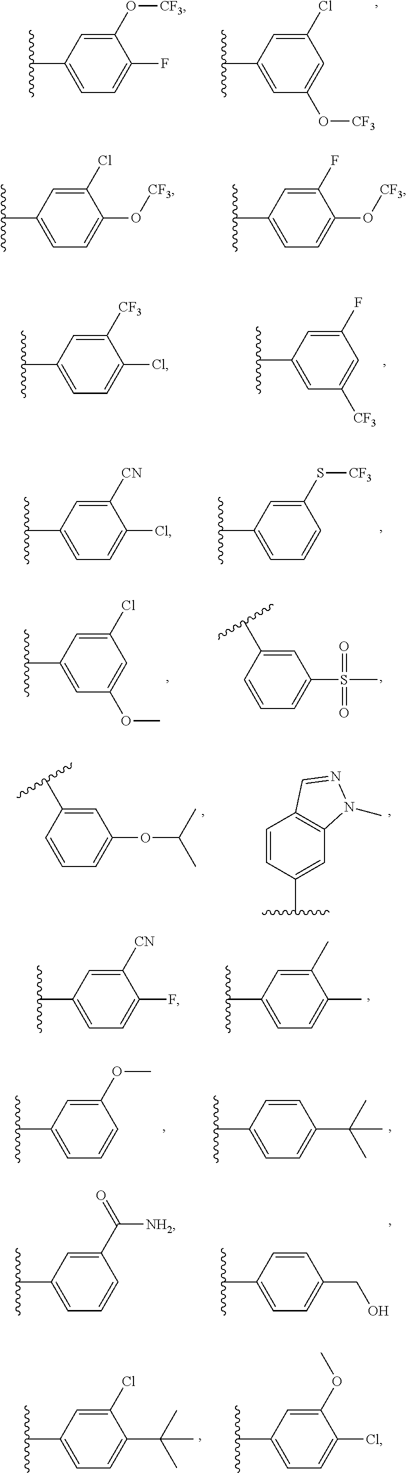

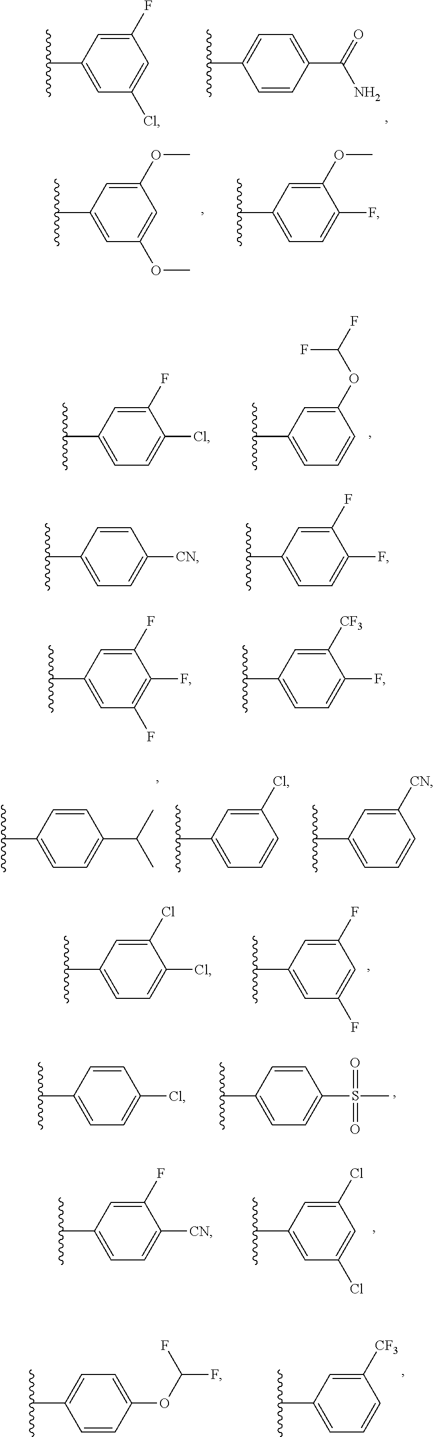

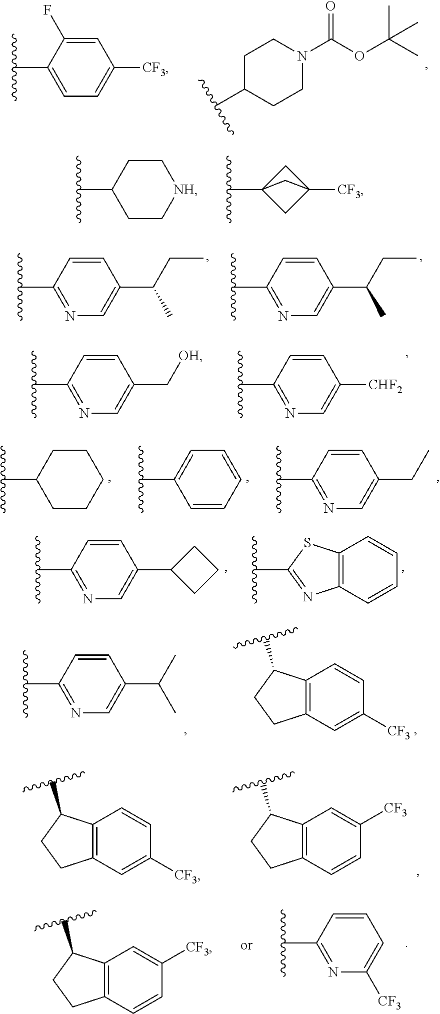

In some embodiments, Ring A is selected from those depicted in Table D, below.

As defined generally above, Ring B is

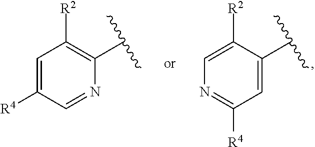

wherein each of R² and R⁴ is as defined herein and as described in embodiments herein, both singly and in combination.

In some embodiments, Ring B is

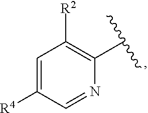

wherein each of R² and R⁴ is as defined herein and described in embodiments herein, both singly and in combination.

In some embodiments, Ring B is

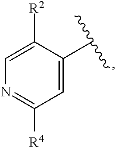

wherein each of R² and R⁴ is as defined herein and described in embodiments herein, both singly and in combination.

In some embodiments, Ring B is

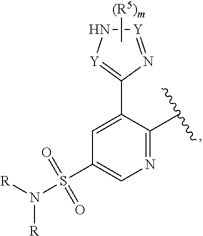

wherein each of R, Y, m, and R⁵ is as defined herein and described in embodiments herein, both singly and in combination. In some embodiments, Ring B is

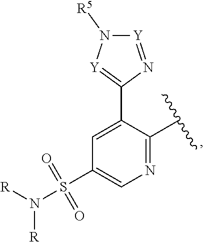

wherein each of Y, R, and R⁵ is as defined herein and described in embodiments herein, both singly and in combination. In some embodiments, Ring B is

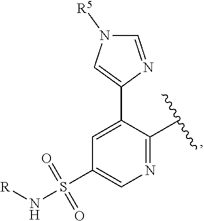

wherein each of R and R⁵ is as defined herein and described in embodiments herein, both singly and in combination.

In some embodiments, Ring B is

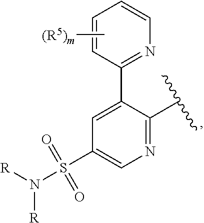

wherein each of m, R, and R⁵ is as defined herein and described in embodiments herein, both singly and in combination. In some embodiments, Ring B is

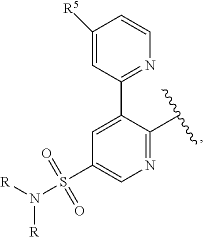

wherein each of R and R⁵ is as defined herein and described in embodiments herein, both singly and in combination. In some embodiments, Ring B is

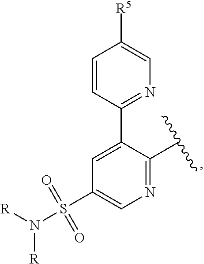

wherein each of R and R⁵ is as defined herein and described in embodiments herein, both singly and in combination. In some embodiments, Ring B is

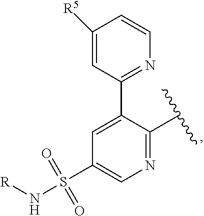

wherein each of R and R is as defined herein and described in embodiments herein, both singly and in combination. In some embodiments, Ring B is

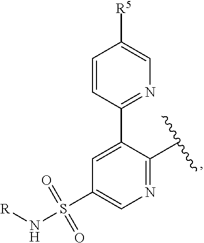

wherein each of R and R⁵ is as defined herein and described in embodiments herein, both singly and in combination.

In some embodiments, Ring B is

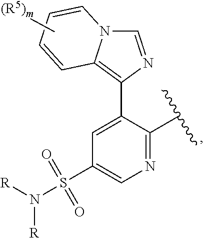

wherein each of m, R, and R⁵ is as defined herein and described in embodiments herein, both singly and in combination. In some embodiments, Ring B is

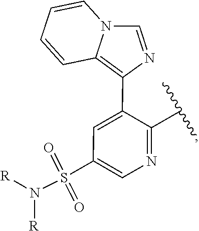

wherein each R is independently as defined herein and described in embodiments herein. In some embodiments, Ring B is

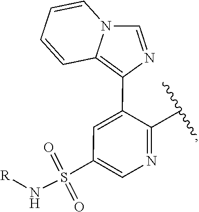

wherein R is as defined above and described in embodiments herein.

In some embodiments, Ring B is

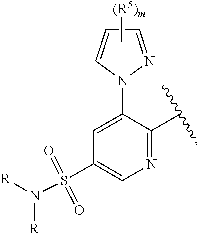

wherein each of m, R, and R⁵ is as defined herein and described in embodiments herein, both singly and in combination. In some embodiments, Ring B is

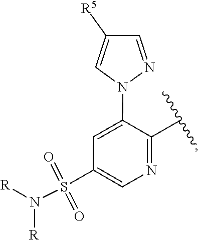

wherein each of R and R⁵ is as defined herein and described in embodiments herein, both singly and in combination. In some embodiments, Ring B is

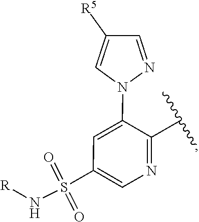

wherein each of R and R⁵ is as defined herein and described in embodiments herein, both singly and in combination.

In some embodiments, Ring B is

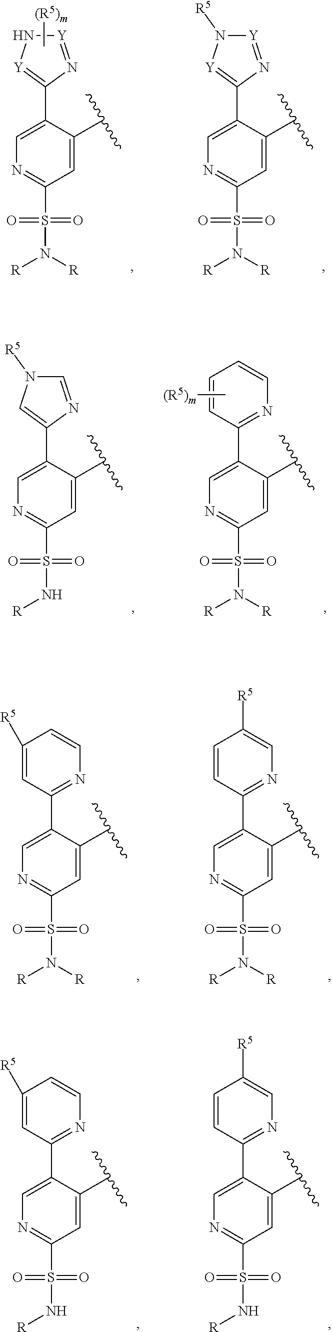

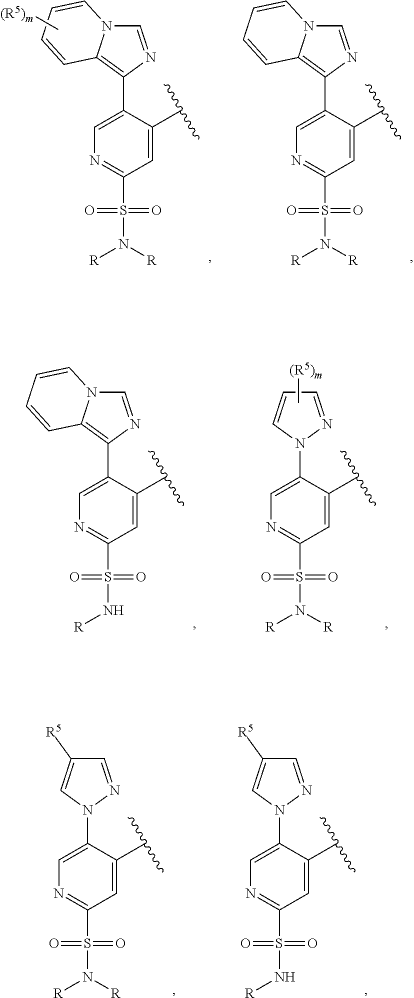

wherein each of R, Y, m, and R⁵ is as defined herein and described in embodiments herein, both singly and in combination.

In some embodiments, Ring B is selected from those depicted in Table D, below.

As defined generally above, each R² is independently selected from —OR, —C(O)NR₂, optionally substituted —C₁₋₆ aliphatic,

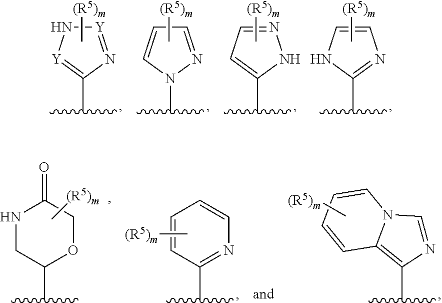

wherein each of Y, m, and R⁵ is as defined herein and described in embodiments herein, both singly and in combination.

In some embodiments, R² is —OR. In some embodiments, R² is —C(O)NR₂. In some embodiments, R² is optionally substituted —C₁₋₆ aliphatic. In some embodiments, R² is

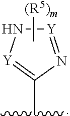

In some embodiments, R² is

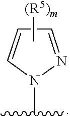

In some embodiments, R² is

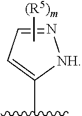

In some embodiments, R² is

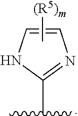

In some embodiments, R² is

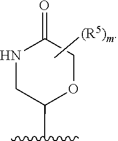

In some embodiments, R² is

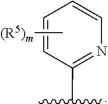

In some embodiments, R² is

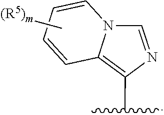

In some embodiments, R² is

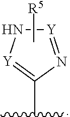

In some embodiments, R² is

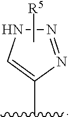

In some embodiments, R² is

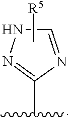

In some embodiments, R² is

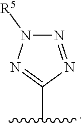

In some embodiments, R² is

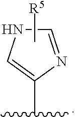

In some embodiments, R² is

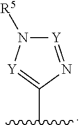

In some embodiments, R² is

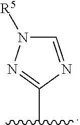

In some embodiments R² is

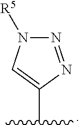

In some embodiments, R² is

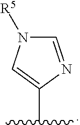

In some embodiments, R² is

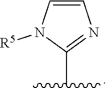

In some embodiments, R² is

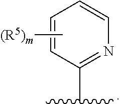

In some embodiments, R² is

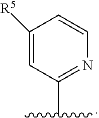

In some embodiments, R² is

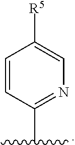

In some embodiments, R² is

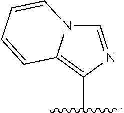 [SMILES: *c1ncn2ccccc12]

In some embodiments, R² is

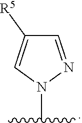

In some embodiments, R² is selected from:

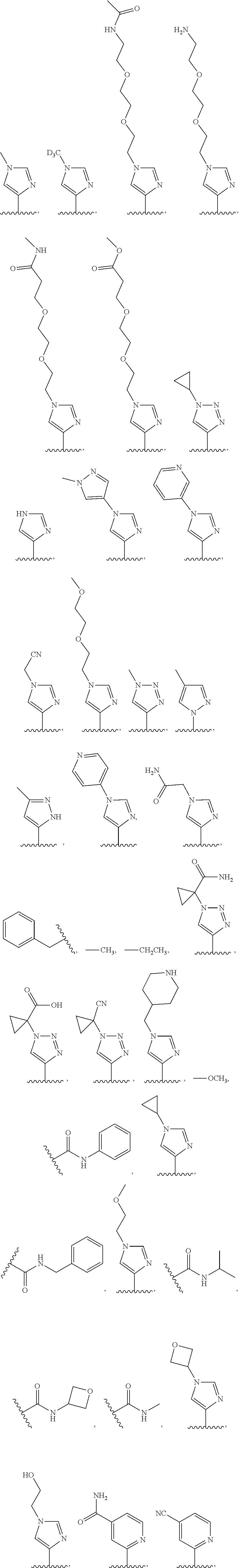

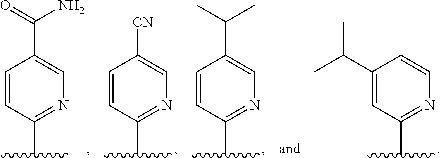

In some embodiments, R² is selected from

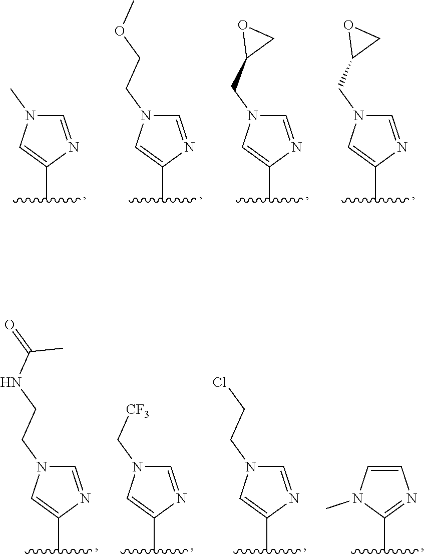

### and —OCH₃.

In some embodiments, R² is selected from those depicted in Table D, below.

As defined generally above, each Y is independently N or CR⁵, wherein R⁵ is as defined herein and as described in embodiments herein.

In some embodiments, Y is N. In some embodiments, Y is CR⁵. In some embodiments, Y is CH.

In some embodiments, both Y are N. In some embodiments, both Y are CR⁵. In some embodiments, both Y are CH. In some embodiments, one Y is N, and the other Y is CR⁵. In some embodiments, one Y is N, and the other Y is CH.

In some embodiments, Y is selected from those depicted in Table D, below.

As defined generally above, each R⁴ is independently —S(O)₂NR₂, —S(O)₂R, —C(O)NR₂, —C(O)R, or optionally substituted —C₁₋₆ aliphatic, wherein each R is independently as defined herein and as described in embodiments herein.

In some embodiments, R⁴ is —S(O)₂NR₂.

In some embodiments, R⁴ is —S(O)₂R.

In some embodiments, R⁴ is —C(O)NR₂.

In some embodiments, R⁴ is —C(O)R.

In some embodiments, R⁴ is -optionally substituted —C₁₋₆ aliphatic.

In some embodiments, R⁴ is selected from

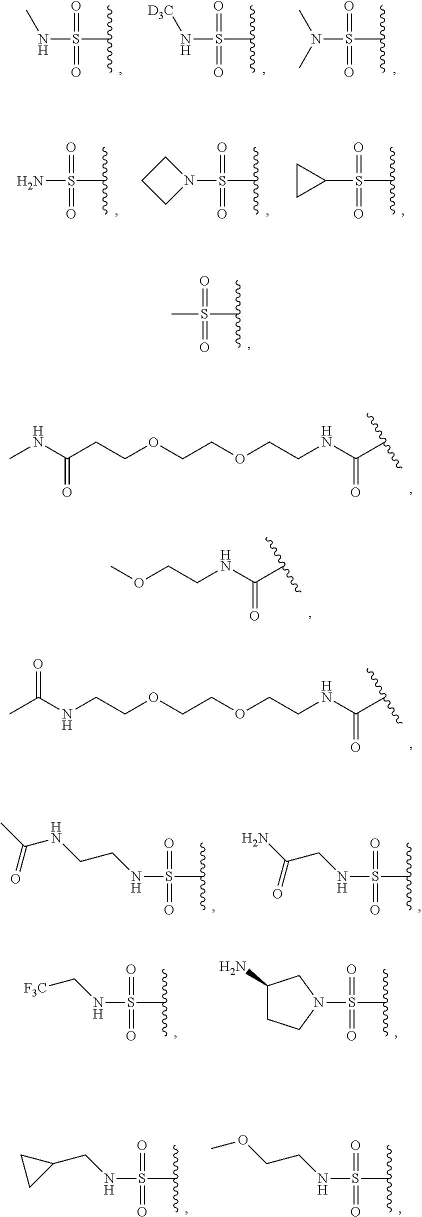

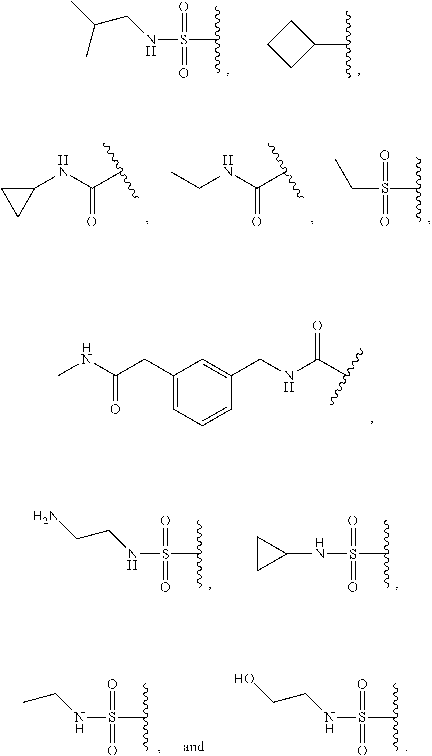

In some embodiments, R⁴ is selected from:

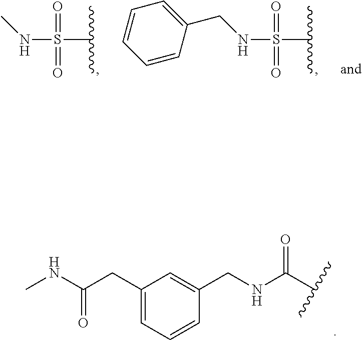

In some embodiments, R⁴ is selected from those depicted in Table D, below.

As defined generally above, each R⁵ is independently R, —CN, —C(O)R, —C(O)NR₂, or optionally substituted 5-6 membered heteroaryl having 1-2 heteroatoms independently selected from nitrogen, oxygen, or sulfur, wherein each R is as defined herein and as described in embodiments herein.

In some embodiments, R⁵ is R.

In some embodiments, R⁵ is —CN.

In some embodiments, R⁵ is —C(O)R.

In some embodiments, R⁵ is —C(O)NR₂.

In some embodiments, R⁵ is optionally substituted 5-6 membered heteroaryl having 1-2 heteroatoms independently selected from nitrogen, oxygen, or sulfur.

In some embodiments, each R⁵ is independently selected from: H, —CH₃, —CD₃,

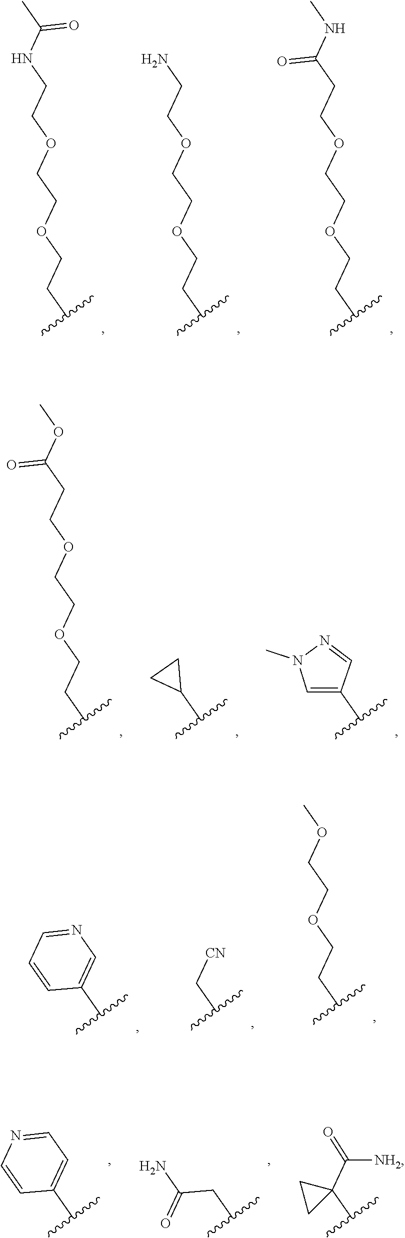

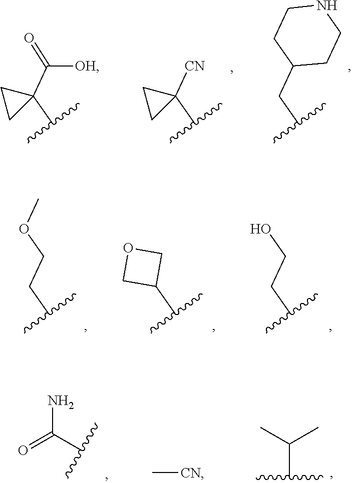

—CH₂CH₃, —C(O)CH₃, —CH₂C(O)NHCH₃,

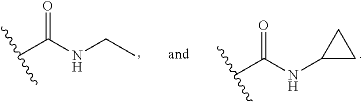

In some embodiments, each R⁵ is independently selected from: —CH₃, —CH₂CH₂OCH₃, —CH₂CF₃, —CH₂CH₂Cl,

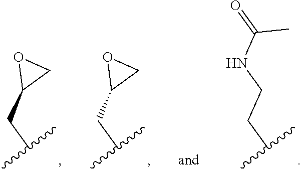

In some embodiments, R⁵ is selected from those depicted in Table D, below.

As defined generally above, each m is independently 0, 1, or 2.

In some embodiments, m is 0. In some embodiments, m is 1. In some embodiments, m is 2.

In some embodiments, m is selected from those depicted in Table D, below.

As defined generally above, each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

In some embodiments, R is H.

In some embodiments, R is optionally substituted —C₁₋₆ aliphatic. In some embodiments, R is unsubstituted —C₁₋₆ aliphatic. In some embodiments, R is —C₁₋₆ aliphatic substituted 1, 2, 3, 4, 5, or 6 times by -halogen, —CN, or —NO₂. In some embodiments, R is —C₁₋₆ aliphatic substituted 1, 2, 3, 4, 5, or 6 times by —F. In some embodiments, R is —C₁₋₃ aliphatic substituted 1, 2, 3, 4, 5, or 6 times by —F. In some embodiments, R is —CH₃. In some embodiments, R is —CH₂CH₃. In some embodiments, R is —CF₃. In some embodiments, R is —CHF₂.

In some embodiments, R is optionally substituted 3-8 membered saturated or partially unsaturated monocyclic carbocyclyl. In some embodiments, R is unsubstituted 3, 4, 5, 6, 7, or 8 membered saturated or partially unsaturated monocyclic carbocyclyl. In some embodiments, R is 3, 4, 5, 6, 7, or 8 membered saturated or partially unsaturated monocyclic carbocyclyl substituted 1, 2, 3, 4, 5, or 6 times by -halogen, —CN, —NO₂, or —C₁₋₆ aliphatic, wherein the —C₁₋₆ aliphatic is optionally substituted 1, 2, 3, 4, 5, or 6 times by -halogen, —CN, or —NO₂. In some embodiments, R is 3, 4, 5, 6, 7, or 8 membered saturated or partially unsaturated monocyclic carbocyclyl substituted 1, 2, 3, 4, 5, or 6 times by -halogen. In some embodiments, R is 3, 4, 5, 6, 7, or 8 membered saturated or partially unsaturated monocyclic carbocyclyl substituted 1, 2, 3, 4, 5, or 6 times by —C₁₋₆ aliphatic, wherein the —C₁₋₆ aliphatic is optionally substituted 1, 2, 3, 4, 5, or 6 times by -halogen. In some embodiments, R is 3, 4, 5, 6, 7, or 8 membered saturated or partially unsaturated monocyclic carbocyclyl substituted 1, 2, 3, 4, 5, or 6 times by —F. In some embodiments, R is 3, 4, 5, 6, 7, or 8 membered saturated or partially unsaturated monocyclic carbocyclyl substituted 1, 2, 3, 4, 5, or 6 times by —C₁₋₆ aliphatic, wherein the —C₁₋₆ aliphatic is optionally substituted 1, 2, 3, 4, 5, or 6 times by —F.

In some embodiments, R is optionally substituted 3-8 membered saturated or partially unsaturated monocyclic heterocyclyl having 1-2 heteroatoms independently selected from nitrogen, oxygen, or sulfur. In some embodiments, R is unsubstituted 3, 4, 5, 6, 7, or 8 membered saturated or partially unsaturated monocyclic heterocyclyl having 1-2 heteroatoms independently selected from nitrogen, oxygen, or sulfur. In some embodiments, R is 3, 4, 5, 6, 7, or 8 membered saturated or partially unsaturated monocyclic heterocyclyl having 1-2 heteroatoms independently selected from nitrogen, oxygen, or sulfur, which is substituted 1, 2, 3, 4, 5, or 6 times by -halogen, —CN, —NO₂, or —C₁₋₆ aliphatic, wherein the —C₁₋₆ aliphatic is optionally substituted 1, 2, 3, 4, 5, or 6 times by -halogen, —CN, or —NO₂. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by -halogen. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by —C₁₋₆ aliphatic, wherein the —C₁₋₆ aliphatic is optionally substituted 1, 2, 3, 4, 5, or 6 times by -halogen. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by —F. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by —C₁₋₆ aliphatic, wherein the —C₁₋₆ aliphatic is optionally substituted 1, 2, 3, 4, 5, or 6 times by —F.

In some embodiments, R is selected from

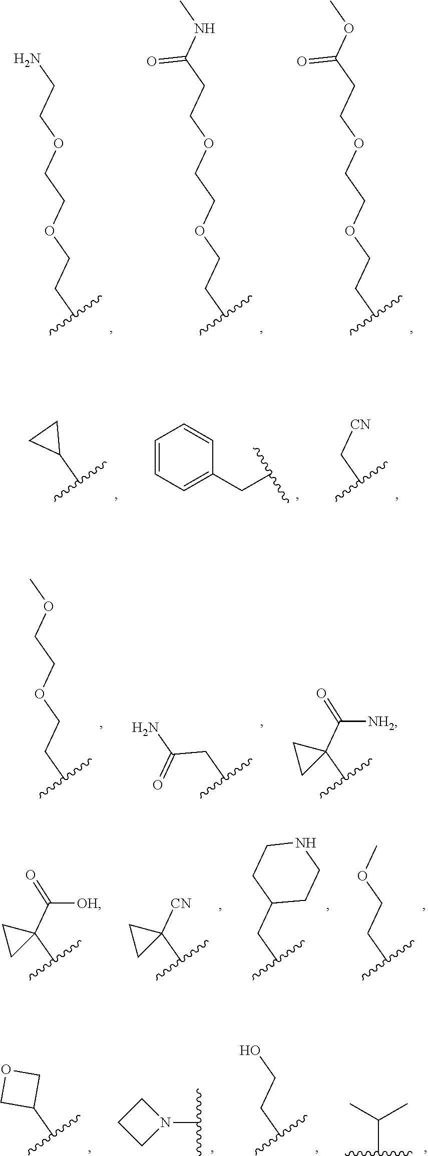

—CH₃, —CD₃, —CH₂CH₃, —CH₂C(O)NHCH₃,

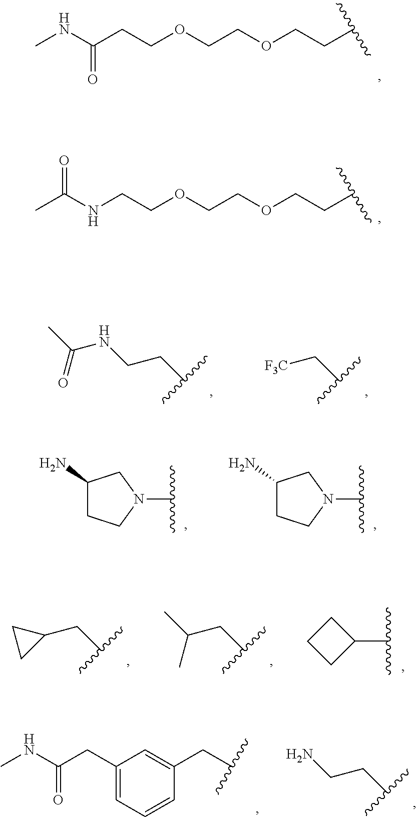

—CH₂CH₂Cl,

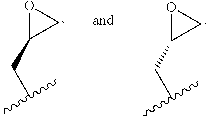 [SMILES: *C[C@@H]1CO1 ; *C[C@H]1CO1]

In some embodiments, R is selected from those depicted in Table D, below.

In some embodiments, a TEAD inhibitor is a compound selected from the following:

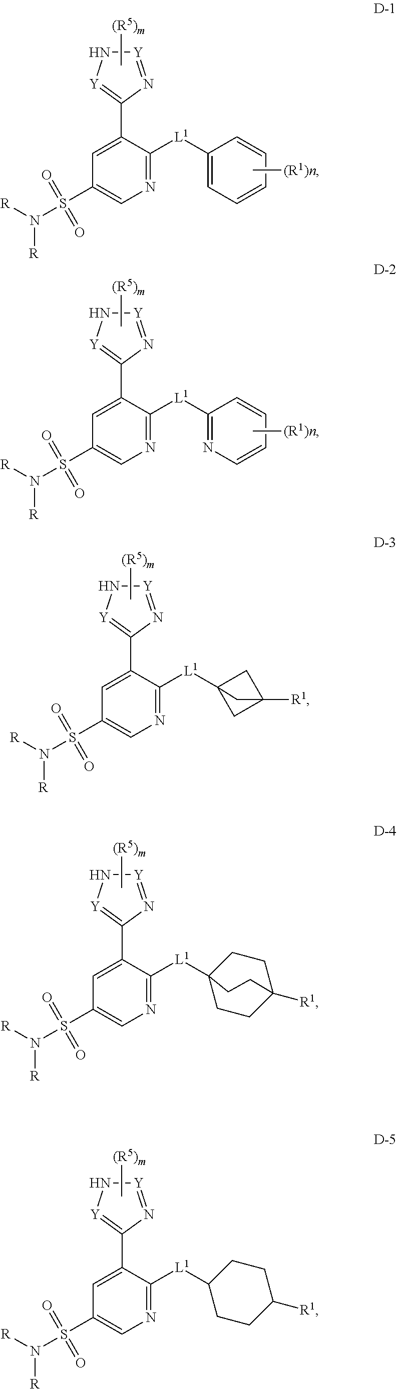

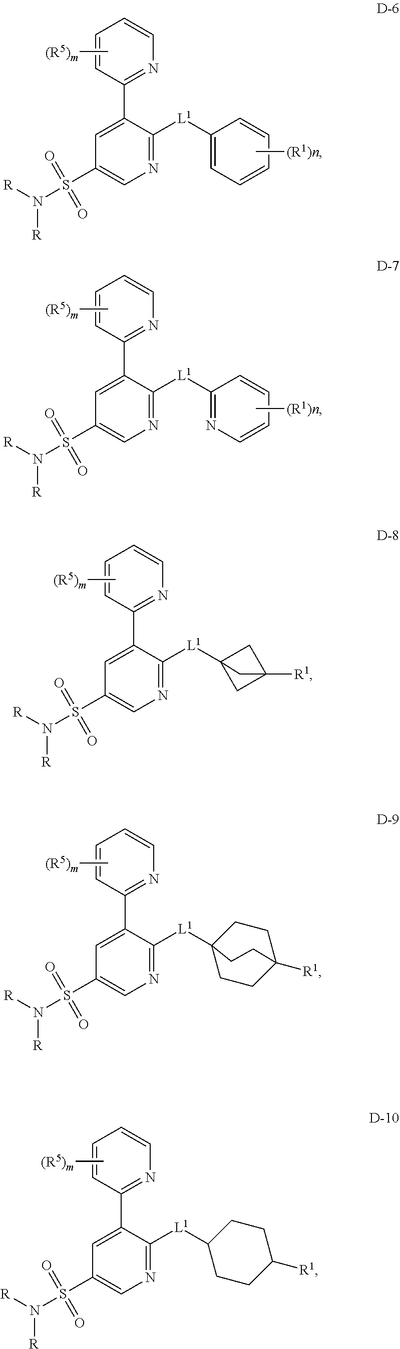

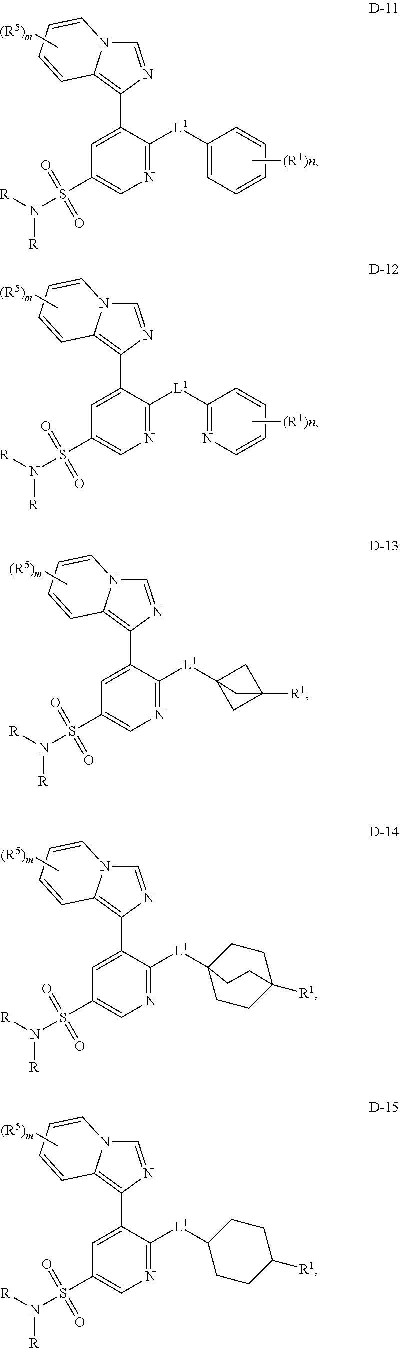

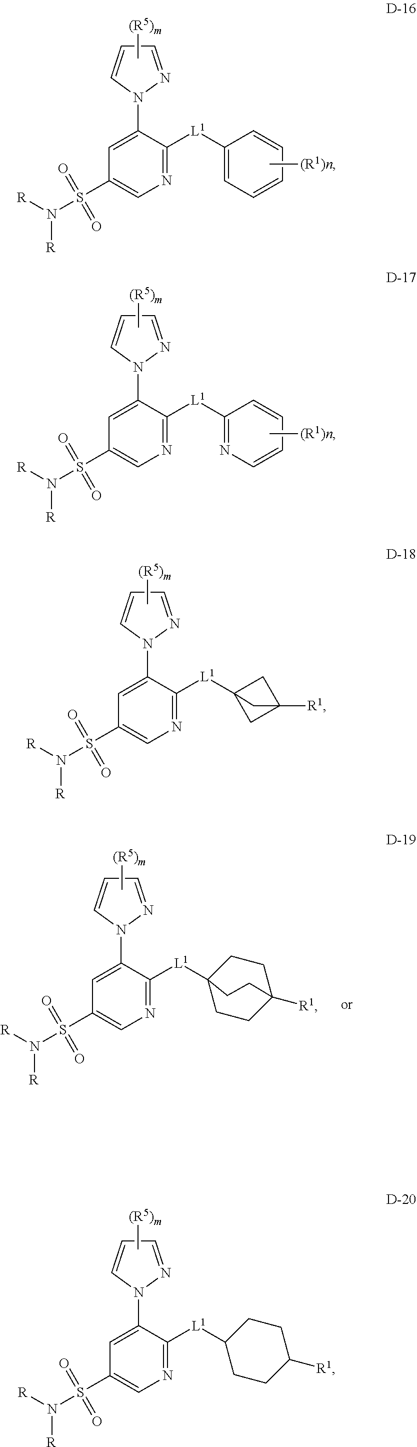

or a pharmaceutically acceptable salt thereof, wherein each of R, R¹, Y, L¹, m, n, and R⁵ is independently as defined above and described in embodiments in the section of TEAD Inhibitors of Formulae D, and D-1 to D-85.

In some embodiments, a TEAD inhibitor is a compound selected from the following

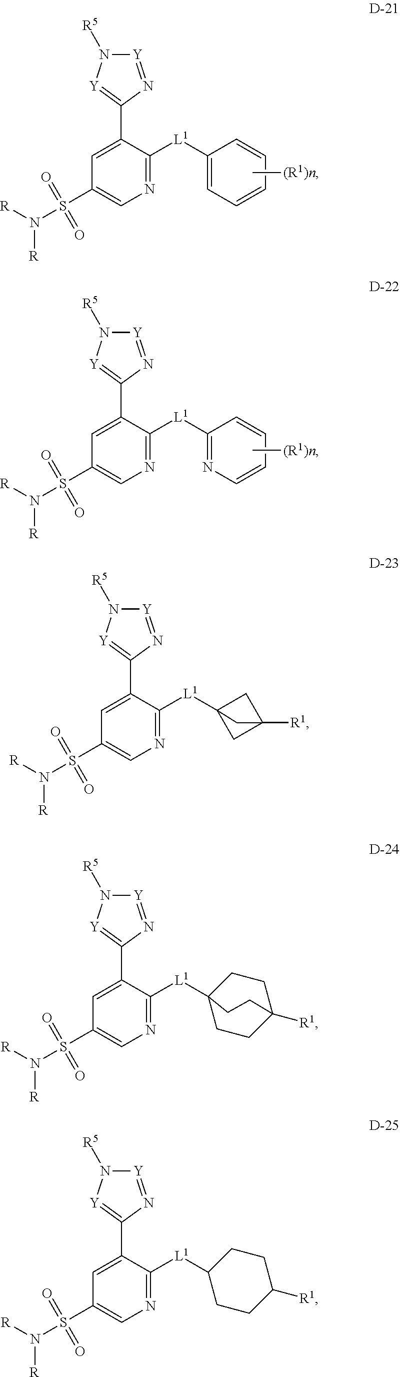

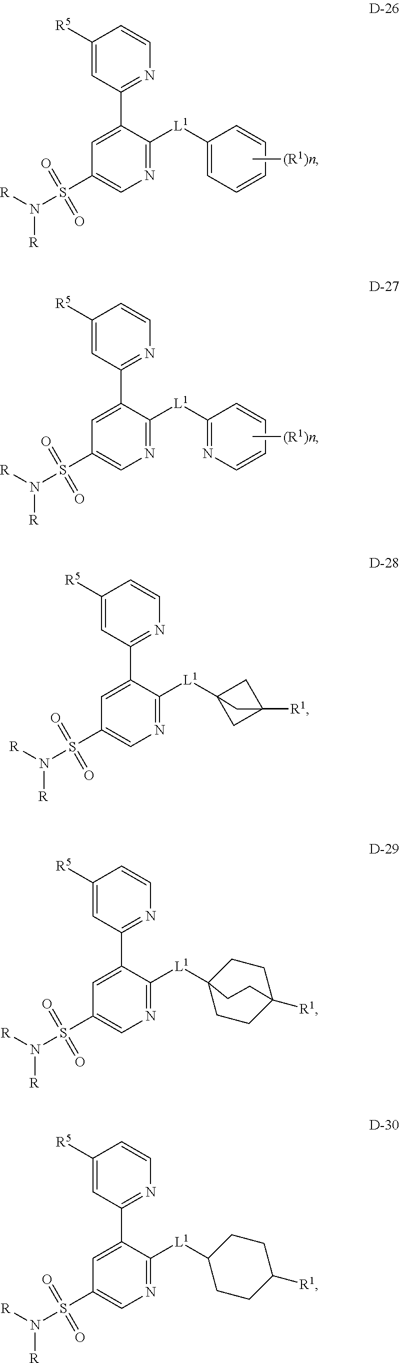

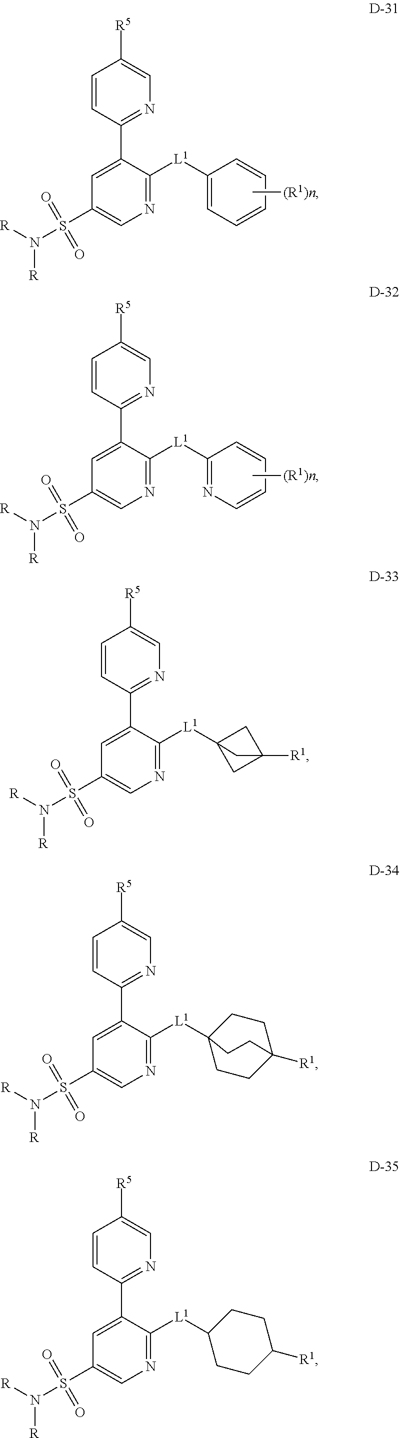

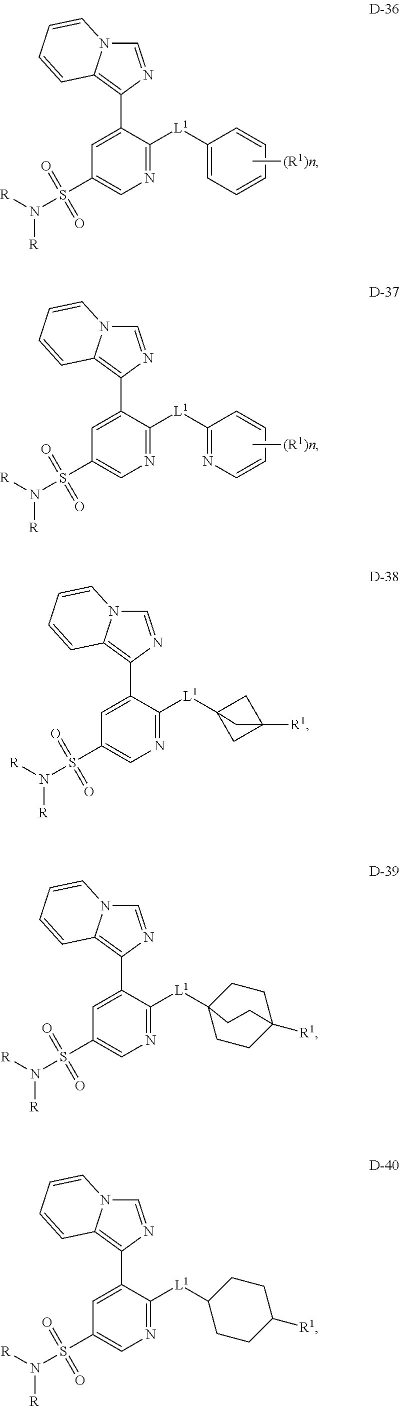

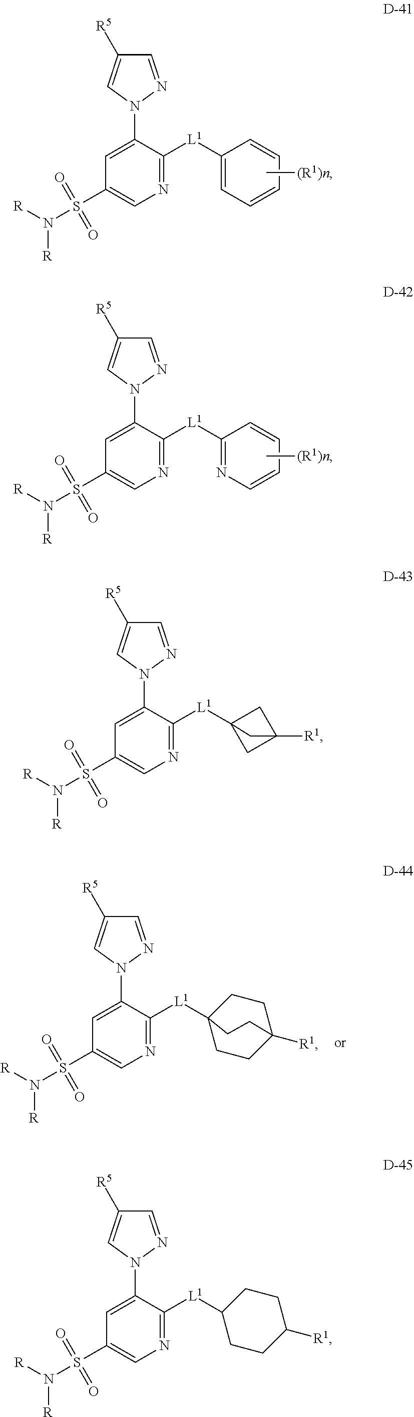

or a pharmaceutically acceptable salt thereof, wherein each of R, R¹, Y, L¹, n, and R⁵ is independently as defined above and described in embodiments in the section of TEAD Inhibitors of Formulae D, and D-1 to D-85.

In some embodiments, a TEAD inhibitor is a compound selected from the following:

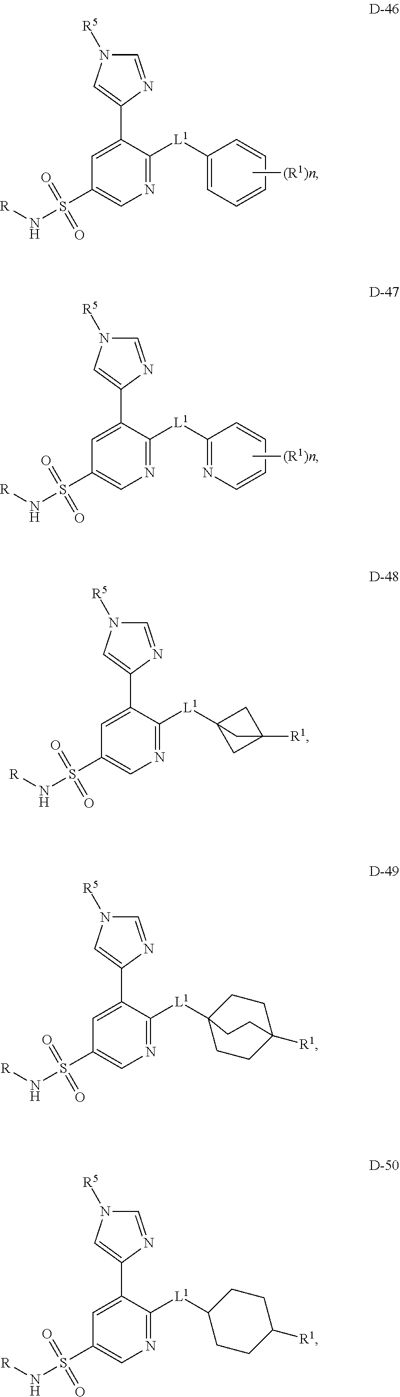

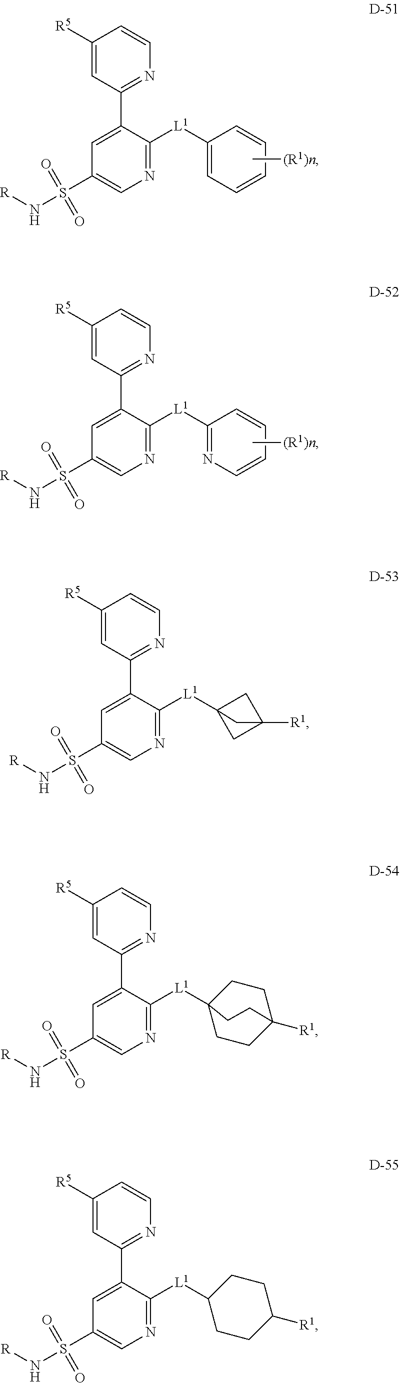

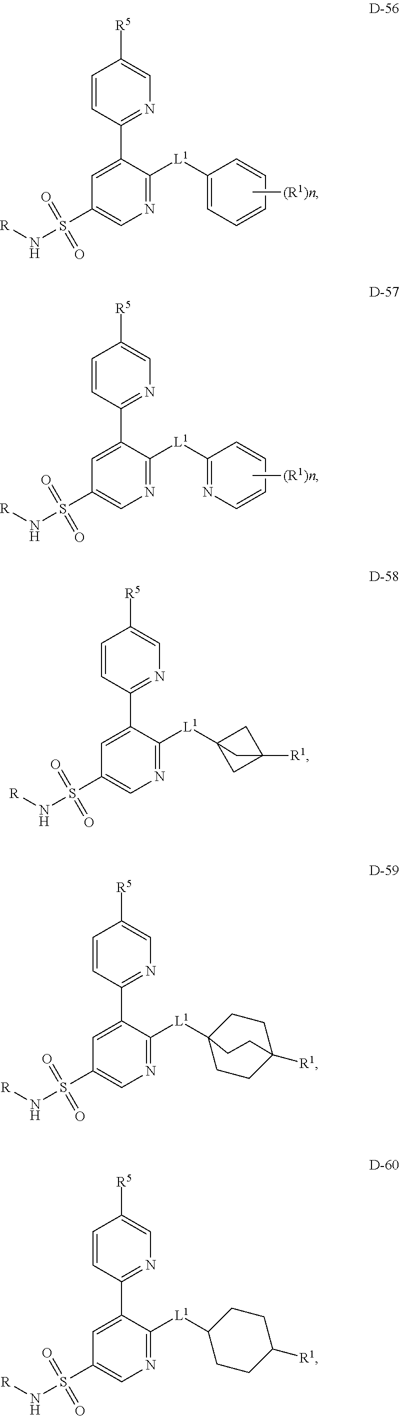

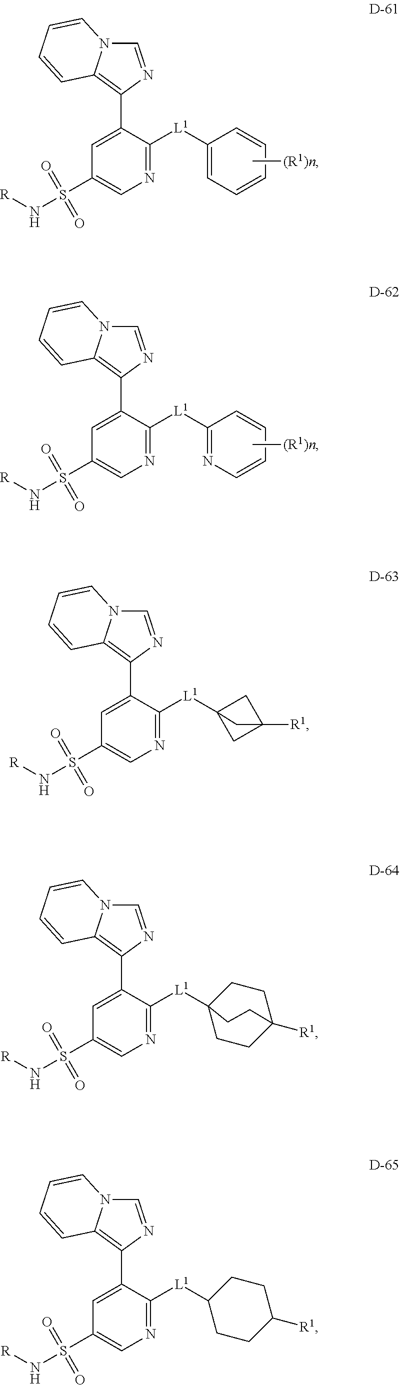

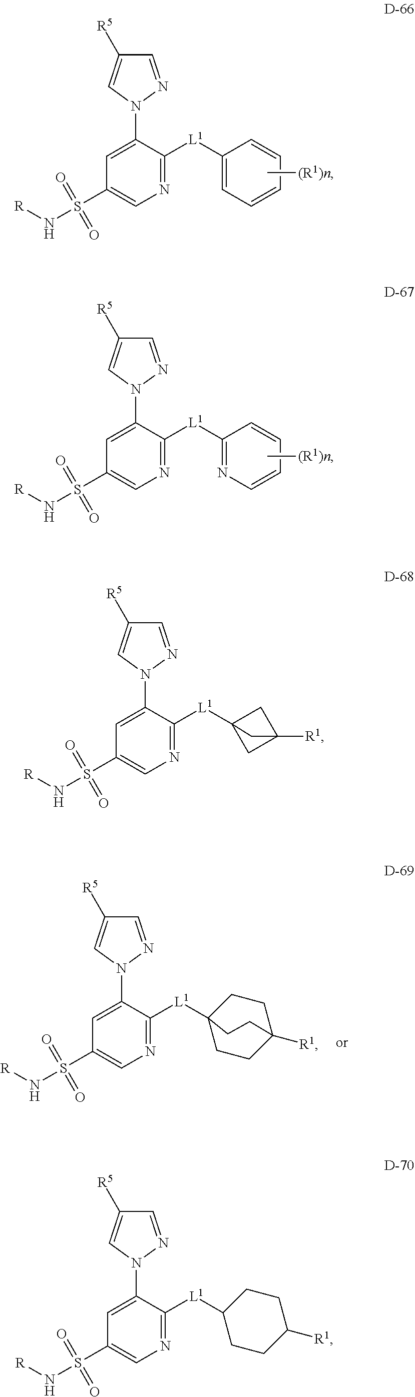

or a pharmaceutically acceptable salt thereof, wherein each of R, R¹, Y, L¹, n, and R⁵ is independently as defined and described in embodiments in the section of TEAD Inhibitors of Formulae D, and D-1 to D-85.

In some embodiments, a TEAD inhibitor is a compound selected from the following:

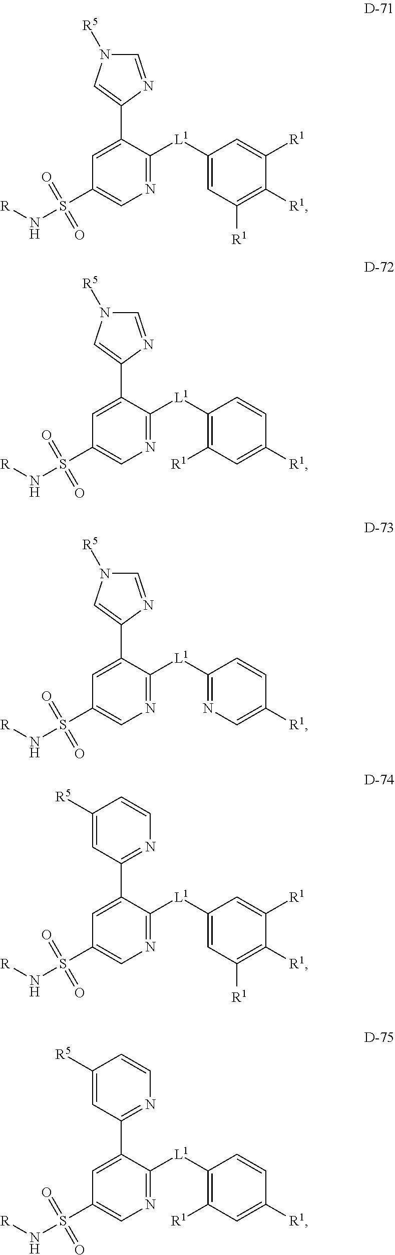

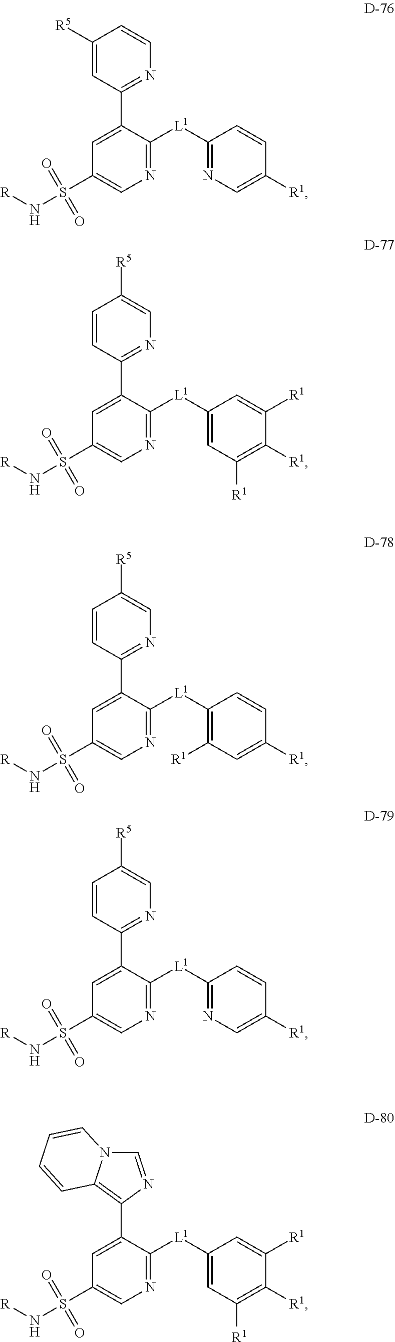

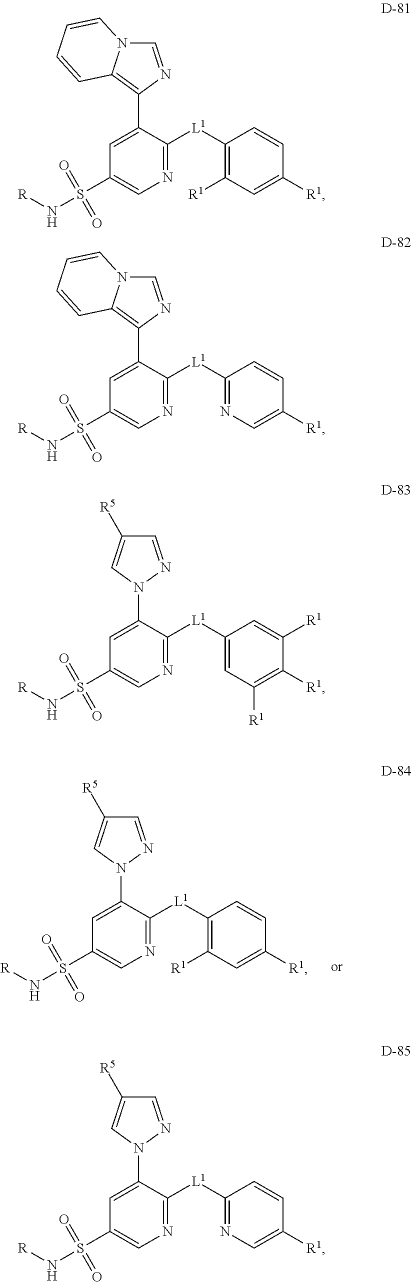

or a pharmaceutically acceptable salt thereof, wherein each of R, R¹, L¹, and R⁵ is independently as defined and described in embodiments in the section of TEAD Inhibitors of Formulae D, and D-1 to D-85.

In some embodiments, a TEAD inhibitor is a compound selected from those listed in Table D, or a pharmaceutically acceptable salt thereof.

TABLE D
Exemplified TEAD Inhibitors
T-D-1

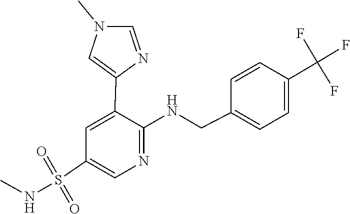 [SMILES: CNS(=O)(=O)c1cnc(NCc2ccc(C(F)(F)F)cc2)c(-c2cn(C)cn2)c1]

T-D-2

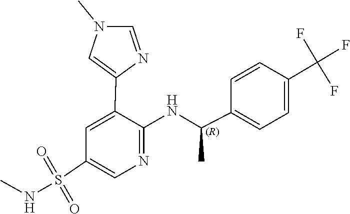

T-D-3

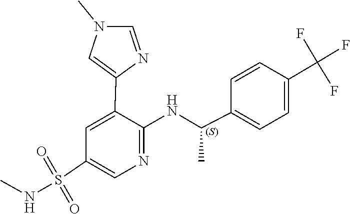

T-D-4

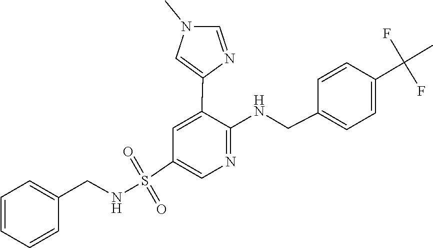 [SMILES: Cn1cnc(-c2cc(S(=O)(=O)NCc3ccccc3)cnc2NCc2ccc(C(C)(F)F)cc2)c1]

T-D-5

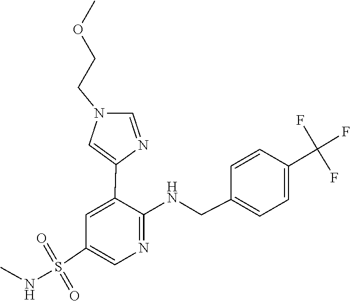 [SMILES: CNS(=O)(=O)c1cnc(NCc2ccc(C(F)(F)F)cc2)c(-c2cn(CCOC)cn2)c1]

T-D-6

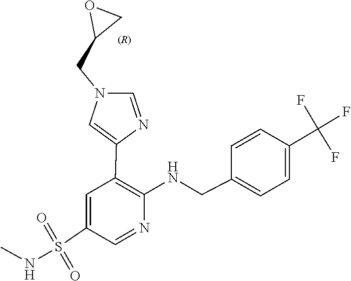

T-D-7

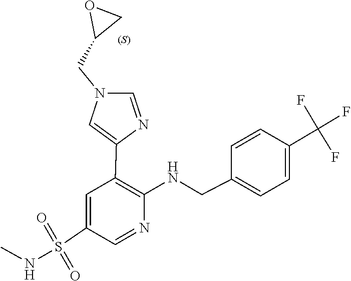

T-D-8

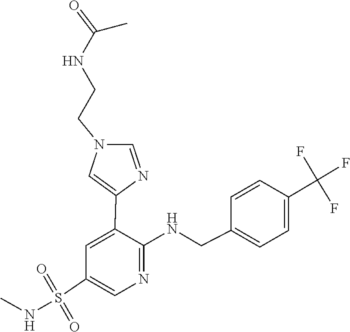 [SMILES: CNS(=O)(=O)c1cnc(NCc2ccc(C(F)(F)F)cc2)c(-c2cn(CCNC(C)=O)cn2)c1]

T-D-9

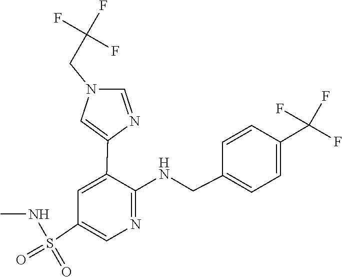 [SMILES: CNS(=O)(=O)c1cnc(NCc2ccc(C(F)(F)F)cc2)c(-c2cn(CC(F)(F)F)cn2)c1]

T-D-10

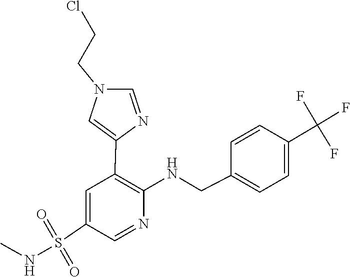 [SMILES: CNS(=O)(=O)c1cnc(NCc2ccc(C(F)(F)F)cc2)c(-c2cn(CCCl)cn2)c1]

T-D-11

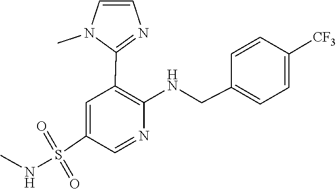 [SMILES: CNS(=O)(=O)c1cnc(NCc2ccc(C(F)(F)F)cc2)c(-c2nccn2C)c1]

T-D-12

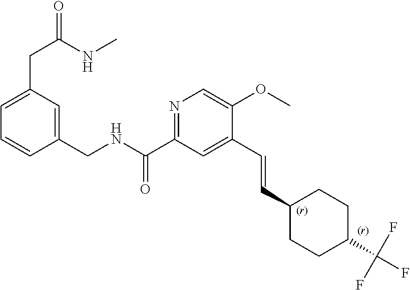

### 5. TEAD Inhibitors of Formulae E, and E-1 to E-204

In certain embodiments, a TEAD inhibitor is a compound of Formula E:

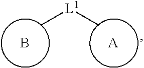

or a pharmaceutically acceptable salt thereof, wherein
L¹ is a covalent bound, or a C₁₋₆ bivalent straight or branched hydrocarbon chain wherein 1, 2, or 3 methylene units of the chain are independently and optionally replaced with —N(R)—, —O—, or —C(O)—;
Ring A is selected from

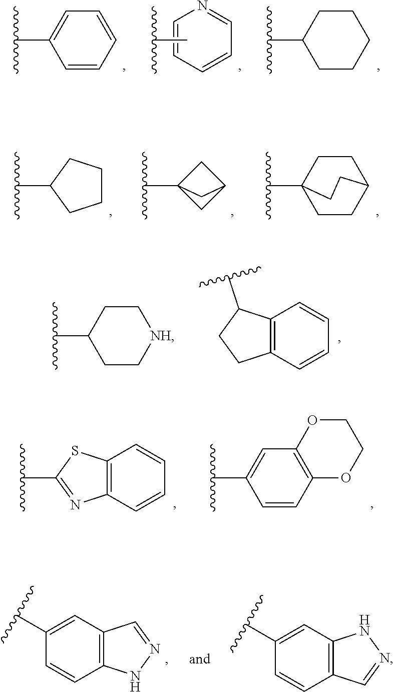

each of which is optionally substituted;
Ring B is selected from

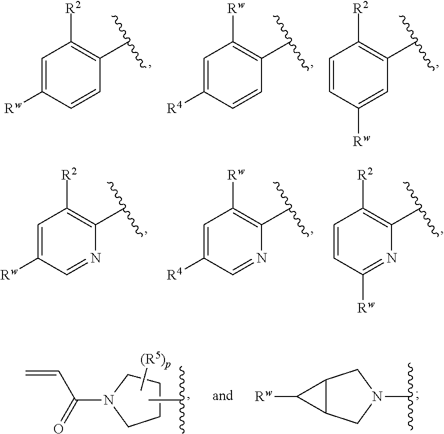

each R^{w} is independently selected from

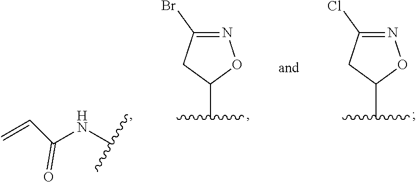

each R² is independently selected from —OR, —C(O)NR₂, optionally substituted —C₁₋₆ aliphatic,

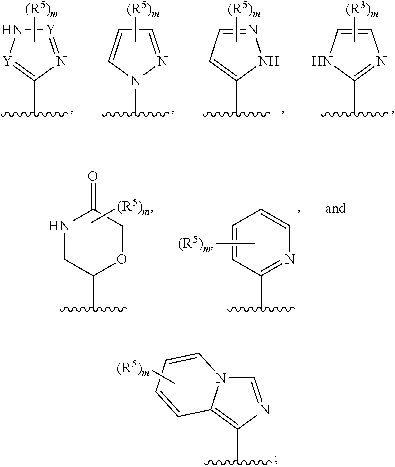

each Y is independently N or CR⁵;
each R³ is independently H or optionally substituted —C₁₋₆ aliphatic;
each R⁴ is independently —S(O)₂NR₂, —S(O)₂R, —C(O)NR₂, —C(O)R, or optionally substituted —C₁₋₆ aliphatic;
each R⁵ is independently R, —CN, —C(O)R, —C(O)NR₂, or optionally substituted 5-6 membered heteroaryl having 1-2 heteroatoms independently selected from nitrogen, oxygen, or sulfur;
each m is independently 0, 1, or 2;
p is 0, 1, or 2, and
each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

As defined generally above, L¹ is a covalent bound, or a C₁₋₆ bivalent straight or branched hydrocarbon chain wherein 1, 2, or 3 methylene units of the chain are independently and optionally replaced with —N(R)—, —O—, or —C(O)—.

In some embodiments, L¹ is a covalent bond.

In some embodiments, L¹ is C₁₋₆ bivalent straight or branched hydrocarbon chain wherein 1, 2, or 3 methylene units of the chain are independently and optionally replaced with —N(R)—.

In some embodiments, L¹ is C₁₋₆ bivalent straight or branched hydrocarbon chain wherein 1, 2, or 3 methylene units of the chain are independently and optionally replaced with —O—.

In some embodiments, L¹ is C₁₋₆ bivalent straight or branched hydrocarbon chain wherein 1, 2, or 3 methylene units of the chain are independently and optionally replaced with —C(O)—.

In some embodiments, L¹ is —NH—. In some embodiments, L¹ is —NH—CH₂—. In some embodiments, L¹ is —NH—CH₂—CH₂—. In some embodiments, L¹ is —CH₂—. In some embodiments, L¹ is

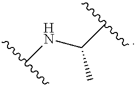 [SMILES: *N[C@H](*)C]

In some embodiments, L¹ is

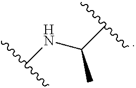 [SMILES: *N[C@@H](*)C]

In some embodiments, L¹ is

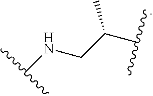 [SMILES: *NC[C@@H](*)C]

In some embodiments, L¹ is

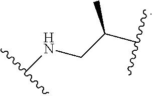 [SMILES: *NC[C@H](*)C]

In some embodiments, L¹ is —CH═CH—. In some embodiments, L¹ is

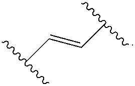 [SMILES: */C=C/*]

In some embodiments, L¹ is

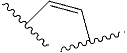 [SMILES: */C=C\*]

In some embodiments, L¹ is —NH—C(O)—.

In some embodiments, L¹ is selected from those depicted in Table E, below.

As defined generally above, Ring A is selected from

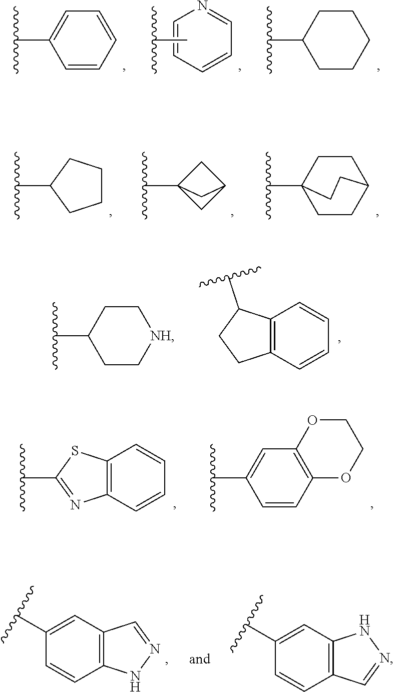

each of which is optionally substituted.

In some embodiments, Ring A is optionally substituted

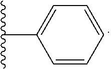 [SMILES: *c1ccccc1]

In some embodiments, Ring A is optionally substituted

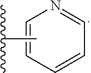

In some embodiments, Ring A is optionally substituted

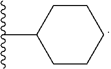 [SMILES: *C1CCCCC1]

In some embodiments, Ring A is optionally substituted

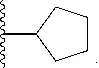 [SMILES: *C1CCCC1]

In some embodiments, Ring A is optionally substituted

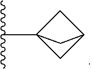 [SMILES: *C12CC(C1)C2]

In some embodiments, Ring A is optionally substituted

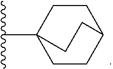 [SMILES: *C12CCC(CC1)CC2]

In some embodiments, Ring A is optionally substituted

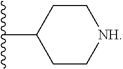 [SMILES: *C1CCNCC1]

In some embodiments, Ring A is optionally substituted

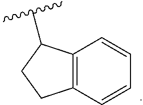 [SMILES: *C1CCc2ccccc21]

In some embodiments, Ring A is optionally substituted

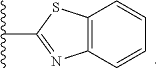 [SMILES: *c1nc2ccccc2s1]

In some embodiments, Ring A is optionally substituted

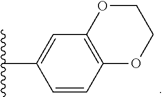 [SMILES: *c1ccc2c(c1)OCCO2]

In some embodiments, Ring A is optionally substituted

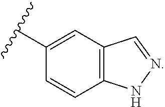 [SMILES: *c1ccc2[nH]ncc2c1]

In some embodiments, Ring A is optionally substituted

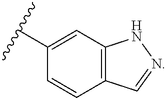 [SMILES: *c1ccc2cn[nH]c2c1]

In some embodiments, Ring A is selected from

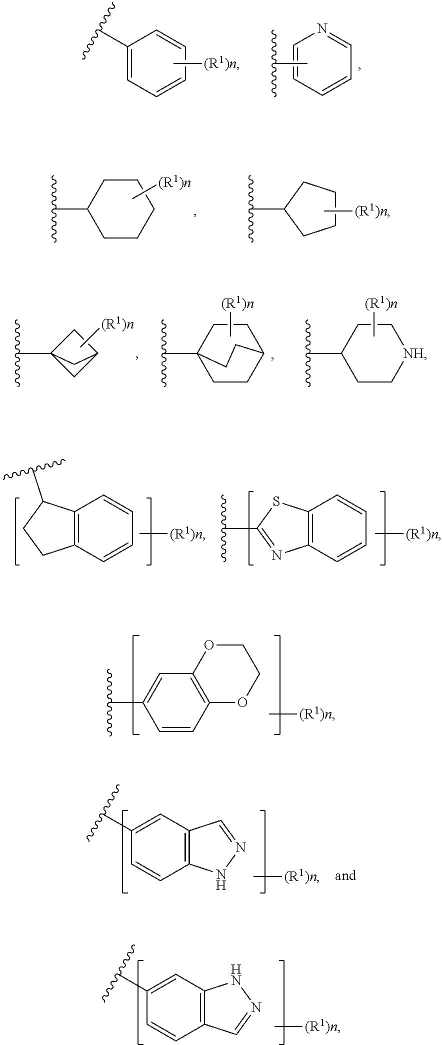

wherein each R¹ is independently R, halogen, —CN, —C(O)R, —C(O)NR₂, —OR, —SR, —S(O)₂NR₂, or —S(O)₂R, and each n is independently 0, 1, 2, or 3, wherein each R is independently as defined herein and as described in embodiments herein.

In some embodiments, R¹ is R. In some embodiments, R¹ is halogen. In some embodiments, R¹ is —CN. In some embodiments, R¹ is —C(O)R. In some embodiments, R¹ is —C(O)NR₂. In some embodiments, R¹ is —OR. In some embodiments, R¹ is —SR. In some embodiments, R¹ is —S(O)₂NR₂. In some embodiments, R¹ is —S(O)₂R.

In some embodiments, each R¹ is independently H, halogen, —C₁₋₆ aliphatic optionally substituted by 1-6 halogen, 3-8 membered saturated or partially unsaturated monocyclic carbocyclyl optionally substituted by 1-6 halogen, or 3-8 membered saturated or partially unsaturated monocyclic heterocyclyl having 1-2 heteroatoms independently selected from nitrogen, oxygen, or sulfur optionally substituted by 1-6 halogen.

In some embodiments, each R¹ is independently H, —CF₃, —C(O)NH₂, —CH₃, —CH₂CH₃, —OCH₃, —CHF₂, —OCF₃, —OCHF₂, —SCF₃, —Cl, —S(O)₂—NH₂, —OCH₂CH₃, —F, —C(O)NHCH₃, —CN, —S(O)₂—CH₃, —OCH(CH₃)₂, —CH(CH₃)₂, —C(CH₃)₃, —CH₂OH,

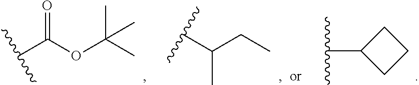

In some embodiments, each R¹ is independently selected from those depicted in Table E, below.

In some embodiments, n is 0. In some embodiments, n is 1. In some embodiments, n is 2. In some embodiments, n is 3.

In some embodiments, Ring A is selected from R¹,

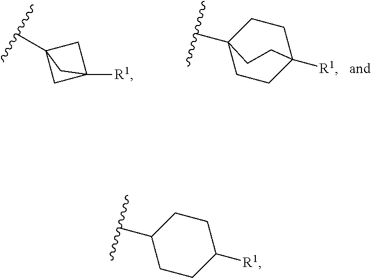

wherein each of R¹ is as defined above and described in embodiments herein, both singly and in combination.

In some embodiments, Ring A is selected from

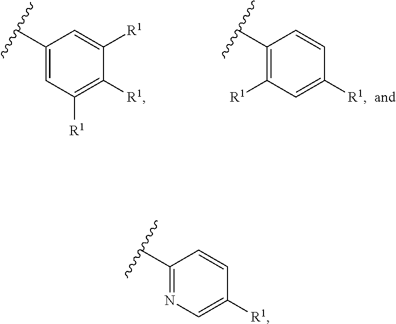

wherein each R¹ is as defined above and described in embodiments herein, both singly and in combination.

In some embodiments, Ring A is

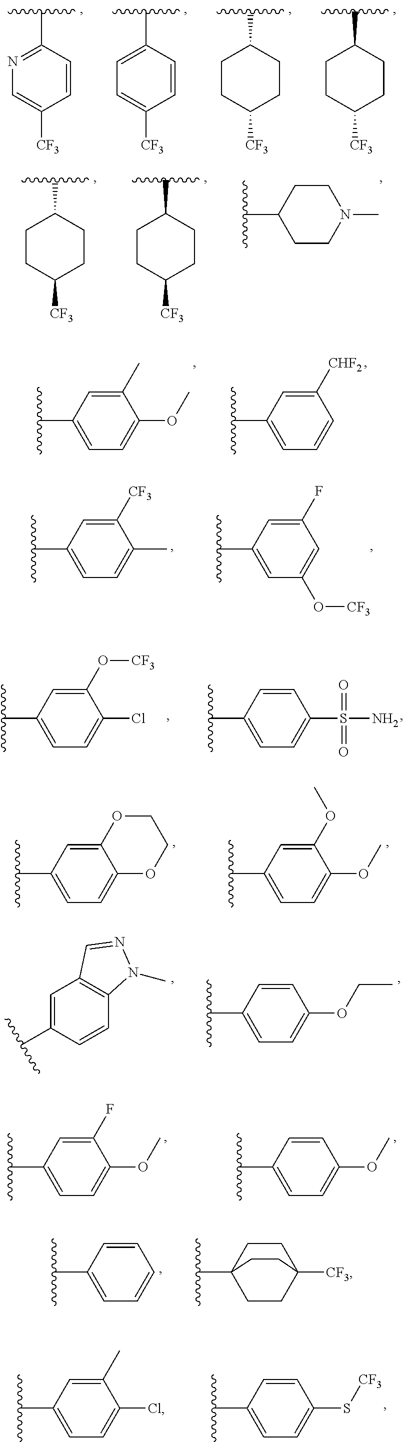

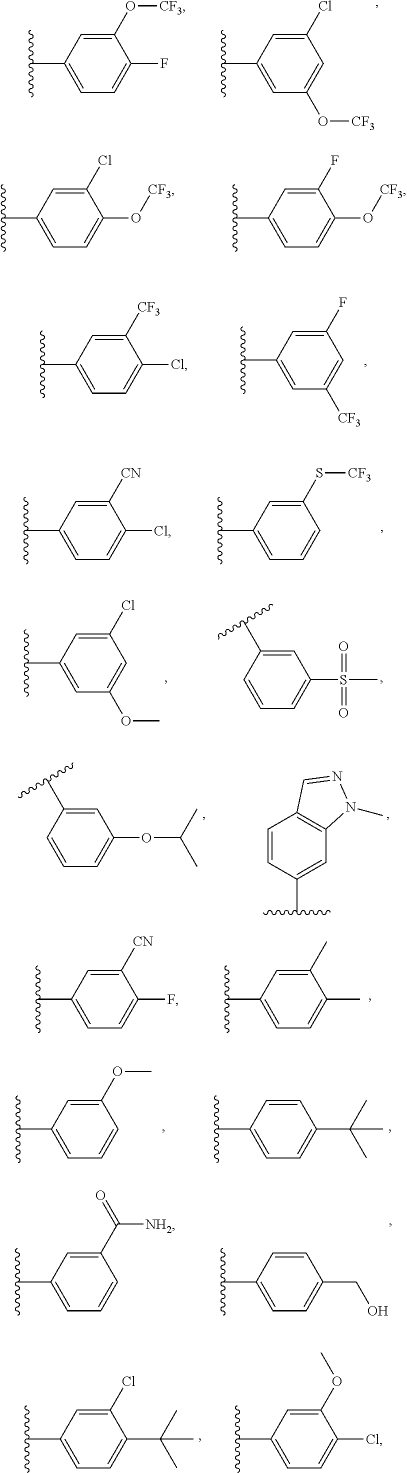

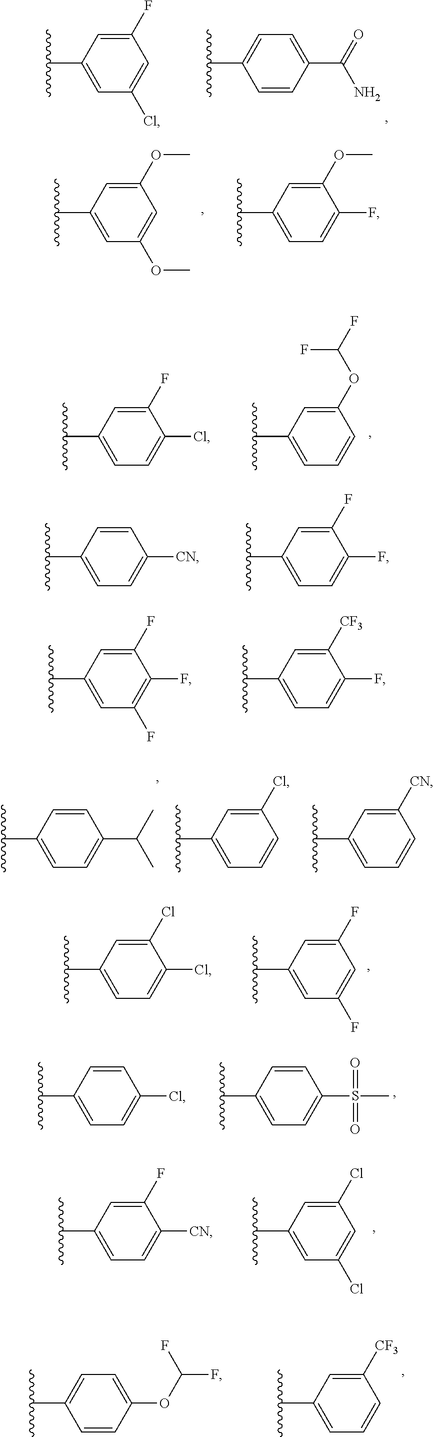

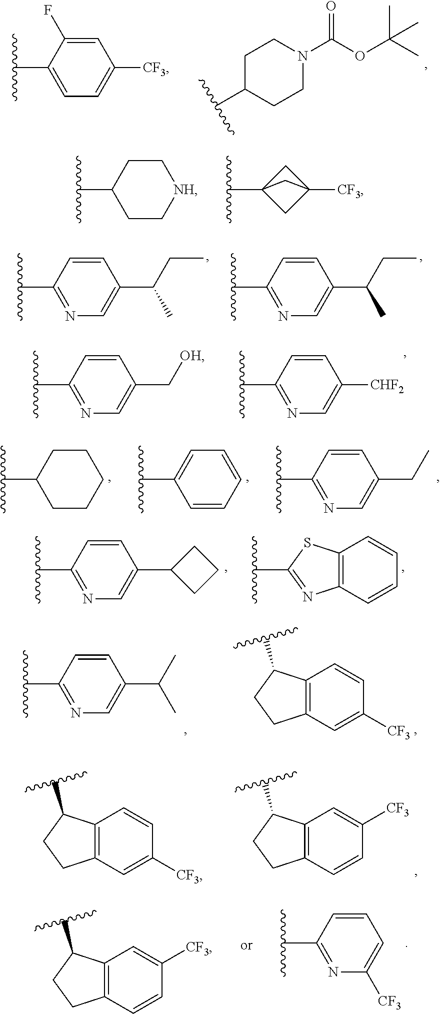

In some embodiments, Ring A is selected from

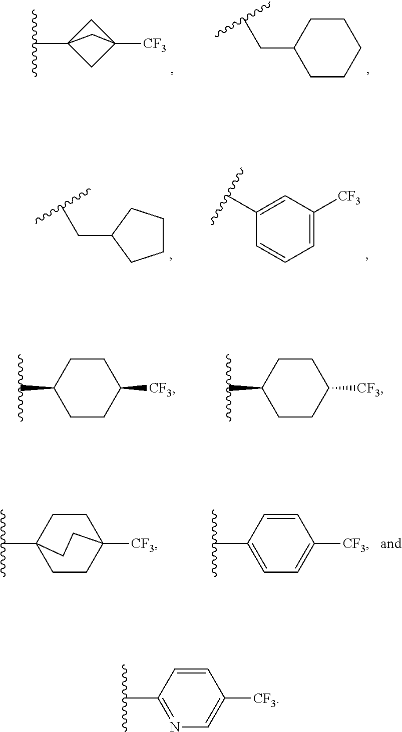

In some embodiments, Ring A is selected from those depicted in Table E, below.

As defined generally above, Ring B is selected from

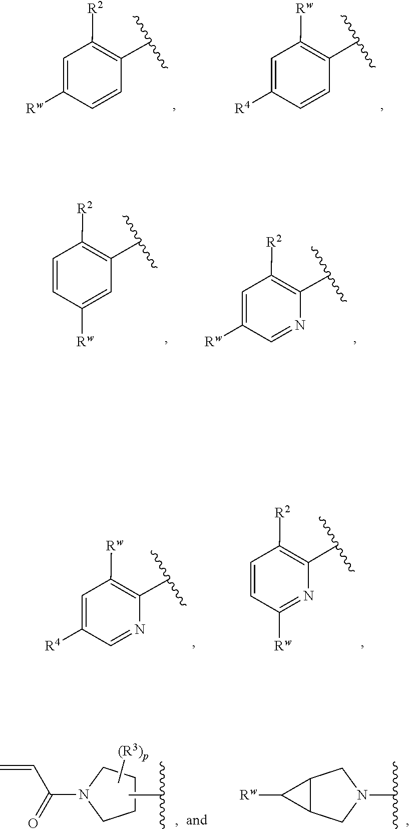

wherein each of R², R³, R^{w}, p, and R⁴ is as defined herein and described in embodiments herein, both singly and in combination.

In some embodiments, Ring B is

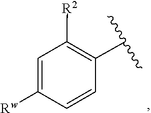

wherein each of R² and R^{w} is as defined herein and described in embodiments herein, both singly and in combination.

In some embodiments, Ring B is

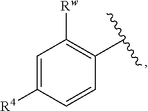

wherein each of R⁴ and R^{w} is as defined herein and described in embodiments herein, both singly and in combination.

In some embodiments, Ring B is

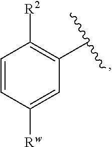

wherein each of R² and R^{w} is as defined herein and described in embodiments herein, both singly and in combination.

In some embodiments, Ring B is

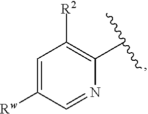

wherein each of R² and R^{w} is as defined herein and described in embodiments herein, both singly and in combination.

In some embodiments, Ring B is

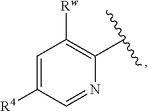

wherein each of R⁴ and R^{w} is as defined herein and described in embodiments herein, both singly and in combination.

In some embodiments, Ring B is

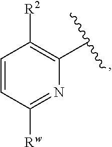

wherein each of R² and R^{w} is as defined herein and described in embodiments herein, both singly and in combination.

In some embodiments, Ring B is

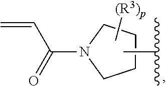

wherein each of R³ and p is as defined herein and described in embodiments herein, both singly and in combination. In some embodiments, Ring B is

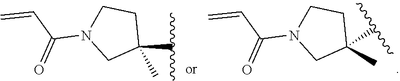 [SMILES: *[C@]1(C)CCN(C(=O)C=C)C1 ; *[C@@]1(C)CCN(C(=O)C=C)C1]

In some embodiments, Ring B is

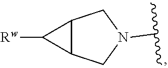

wherein R^{w} is as defined herein and described in embodiments herein. In some embodiments, Ring B is

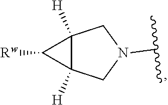

wherein R^{w} is as defined herein and described in embodiments herein.

In some embodiments, Ring B is selected from those depicted in Table E, below.

As defined generally above, R^{w} is selected from

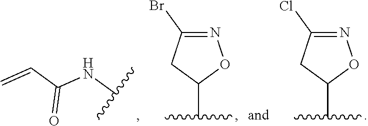

In some embodiments, R^{w} is

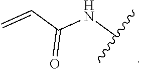 [SMILES: *NC(=O)C=C]

In some embodiments, R^{w} is

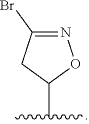 [SMILES: *C1CC(Br)=NO1]

In some embodiments, R^{w} is

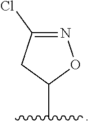 [SMILES: *C1CC(Cl)=NO1]

In some embodiments, R^{w} is

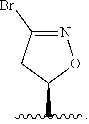 [SMILES: *[C@H]1CC(Br)=NO1]

In some embodiments, R^{w} is

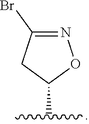 [SMILES: *[C@@H]1CC(Br)=NO1]

In some embodiments, R^{w} is

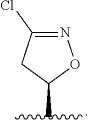 [SMILES: *[C@H]1CC(Cl)=NO1]

In some embodiments R^{w} is

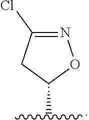 [SMILES: *[C@@H]1CC(Cl)=NO1]

In some embodiments, R^{w} is selected from those depicted in Table E, below.

As defined generally above, each R² is independently selected from —OR, —C(O)NR₂, optionally substituted —C₁₋₆ aliphatic,

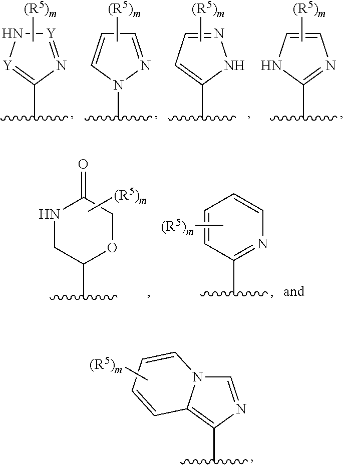

wherein each of Y, m, and R⁵ is as defined herein and described in embodiments herein, both singly and in combination.

In some embodiments, R² is —OR. In some embodiments, R² is —C(O)NR₂. In some embodiments, R² is optionally substituted —C₁₋₆ aliphatic. In some embodiments, R² is

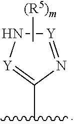

In some embodiments, R² is

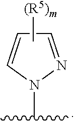

In some embodiments, R² is

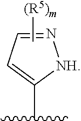

In some embodiments, R² is

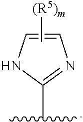

In some embodiments, R² is

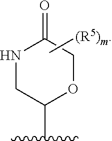

In some embodiments, R² is

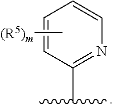

In some embodiments, R² is

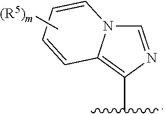

In some embodiments, R² is

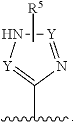

In some embodiments, R² is

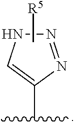

In some embodiments, R² is

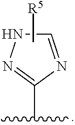

In some embodiments, R² is

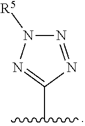

In some embodiments, R² is

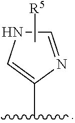

In some embodiments, R² is

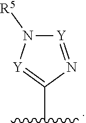

In some embodiments, R² is

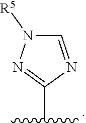

In some embodiments, R² is

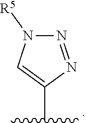

In some embodiments, R² is

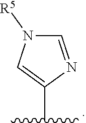

In some embodiments, R² is

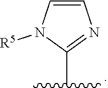

In some embodiments R² is

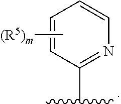

In some embodiments, R² is

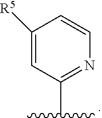

In some embodiments, R² is

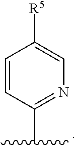

In some embodiments, R² is

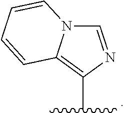 [SMILES: *c1ncn2ccccc12]

In some embodiments, R² is

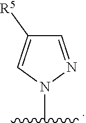

In some embodiments, R² is selected from:

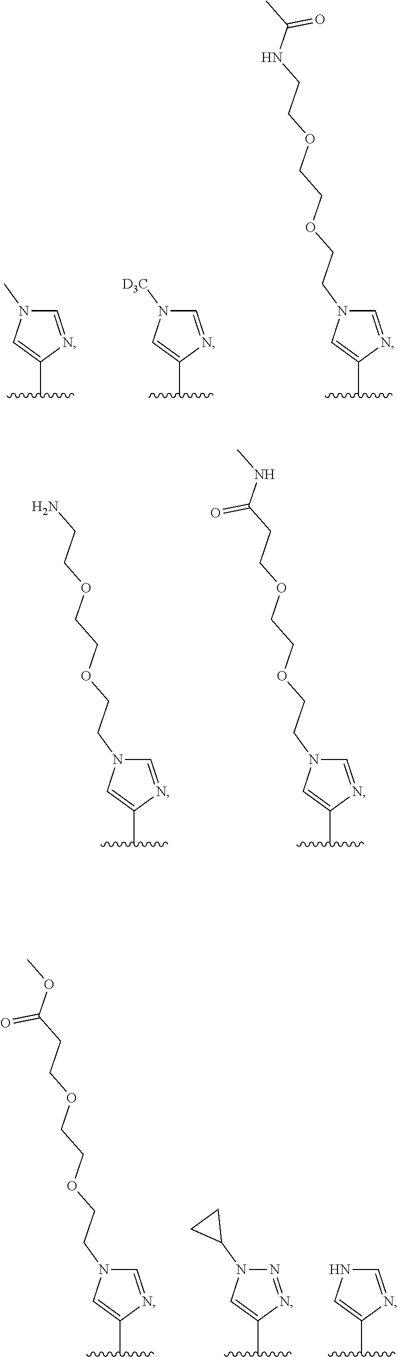

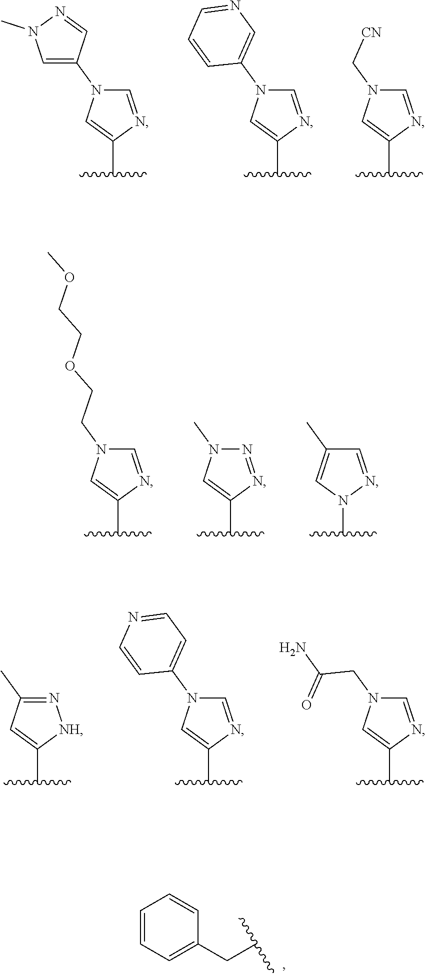

—CH₃, —CH₂CH₃,

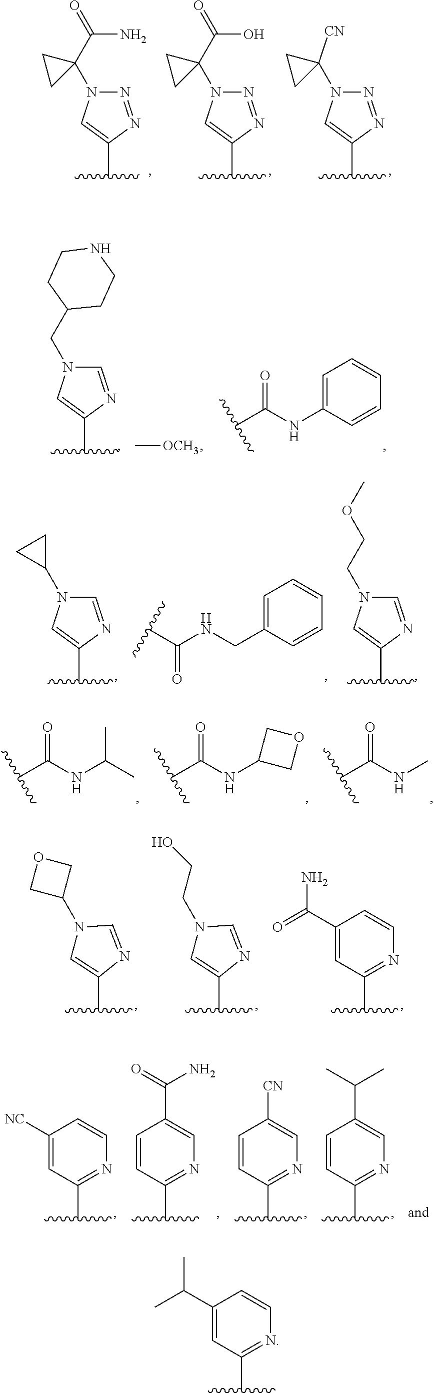

In some embodiments, R² is selected from

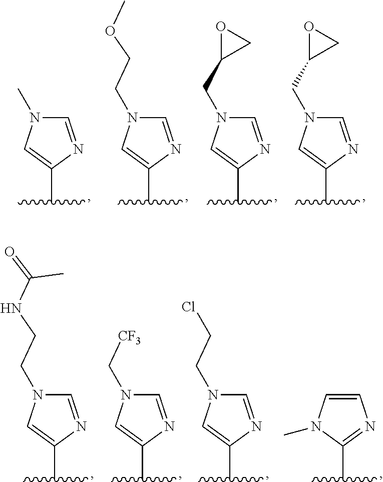

### and —OCH₃.

In some embodiments, R² is selected from those depicted in Table E, below.

As defined generally above, each Y is independently N or CR⁵.

In some embodiments, Y is N. In some embodiments, Y is CR⁵. In some embodiments, Y is CH.

In some embodiments, both Y are N. In some embodiments, both Y are CR⁵. In some embodiments, one Y is N, and the other Y is CR⁵. In some embodiments, both Y are CH. In some embodiments, one Y is N, and the other Y is CH.

In some embodiments, Y is selected from those depicted in Table E, below.

As defined generally above, each R³ is independently H, —C(O)R, or optionally substituted —C₁₋₆ aliphatic, wherein R is as defined herein and described in embodiments herein.

In some embodiments, R³ is H.

In some embodiments, R³ is —C(O)R.

In some embodiments, R³ is optionally substituted —C₁₋₆ aliphatic.

In some embodiments, R³ is selected from H, —CH₃, —CH₂CH₃, —C(O)CH₃, and

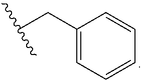 [SMILES: *Cc1ccccc1]

In some embodiments, R³ is selected from those depicted in Table E, below.

As defined generally above, each R⁴ is independently —S(O)₂NR₂, —S(O)₂R, —C(O)NR₂, —C(O)R, or optionally substituted —C₁₋₆ aliphatic, wherein each R is independently as defined herein and as described in embodiments herein.

In some embodiments, R⁴ is —S(O)₂NR₂.

In some embodiments, R⁴ is —S(O)₂R.

In some embodiments, R⁴ is —C(O)NR₂.

In some embodiments, R⁴ is —C(O)R.

In some embodiments, R⁴ is -optionally substituted —C₁₋₆ aliphatic.

In some embodiments, R⁴ is selected from

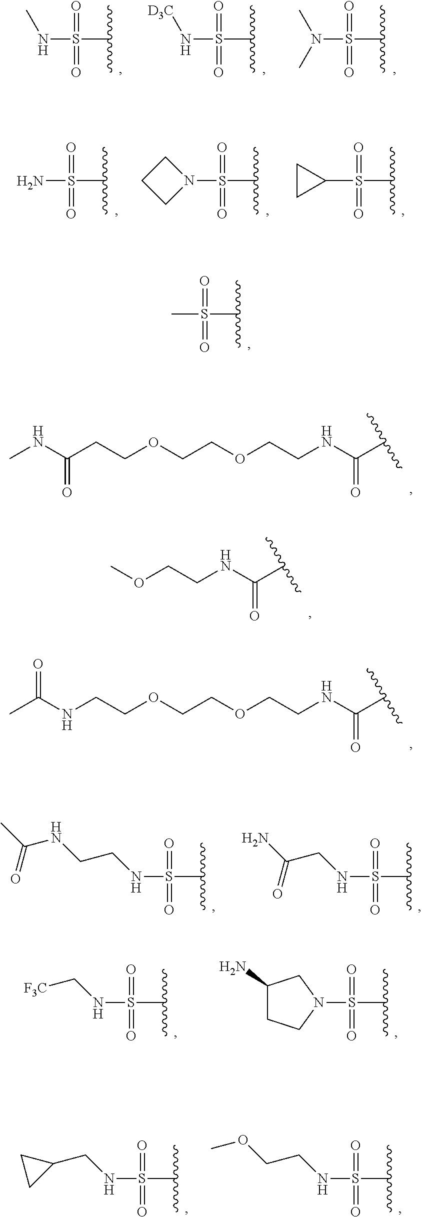

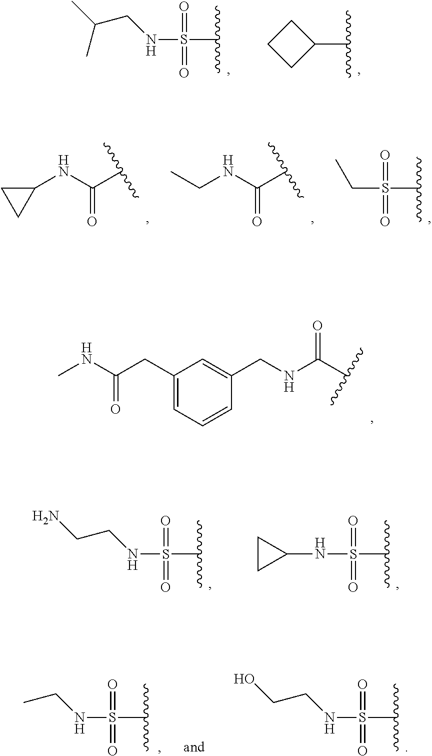

In some embodiments, R⁴ is selected from:

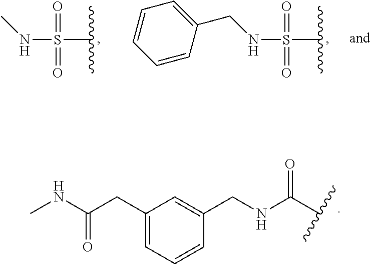

In some embodiments, R⁴ is selected from those depicted in Table E, below.

As defined generally above, each R⁵ is independently R, —CN, —C(O)R, —C(O)NR₂, or optionally substituted 5-6 membered heteroaryl having 1-2 heteroatoms independently selected from nitrogen, oxygen, or sulfur, wherein each R is independently as defined herein and as described in embodiments herein.

In some embodiments, R⁵ is R.

In some embodiments, R⁵ is —CN.

In some embodiments, R⁵ is —C(O)R.

In some embodiments, R⁵ is —C(O)NR₂.

In some embodiments, R⁵ is optionally substituted 5-6 membered heteroaryl having 1-2 heteroatoms independently selected from nitrogen, oxygen, or sulfur.

In some embodiments, each R⁵ is independently selected from: H, —CH₃, —CD₃,

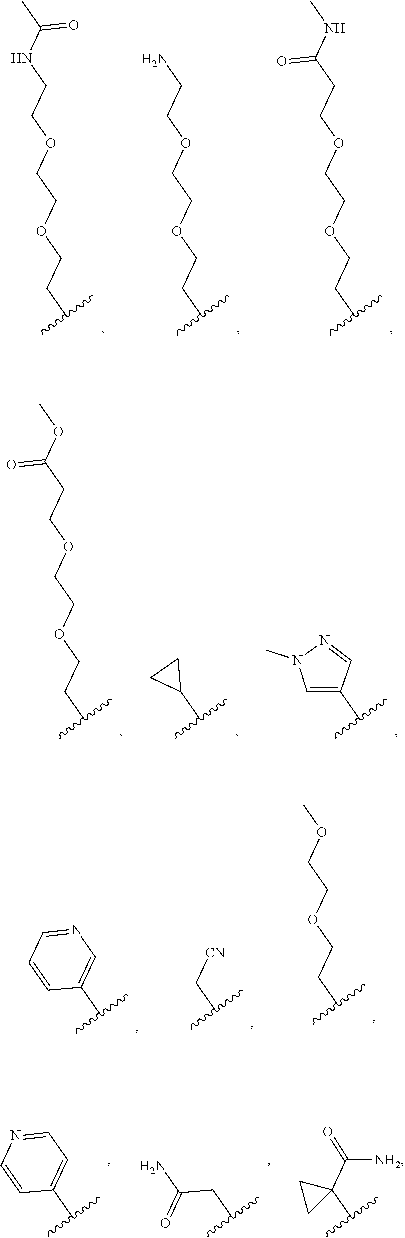

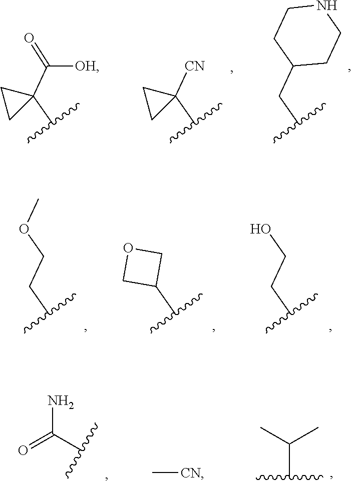

—CH₂CH₃, —C(O)CH₃, —CH₂C(O)NHCH₃,

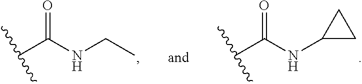

In some embodiments, each R⁵ is independently selected from: —CH₃, —CH₂CH₂OCH₃, —CH₂CF₃, —CH₂CH₂Cl,

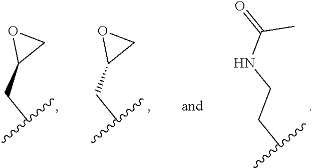

In some embodiments, R⁵ is selected from those depicted in Table E, below.

As defined generally above, each m is independently 0, 1, or 2.

In some embodiments, m is 0. In some embodiments, m is 1. In some embodiments, m is 2.

In some embodiments, m is selected from those depicted in Table E, below.

As defined generally above, p is 0, 1, or 2.

In some embodiments, p is 0. In some embodiments, p is 1. In some embodiments, p is 2.

In some embodiments, p is selected from those depicted in Table E, below.

As defined generally above, each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

In some embodiments, R is H.

In some embodiments, R is optionally substituted —C₁₋₆ aliphatic. In some embodiments, R is unsubstituted —C₁₋₆ aliphatic. In some embodiments, R is —C₁₋₆ aliphatic substituted 1, 2, 3, 4, 5, or 6 times by -halogen, —CN, or —NO₂. In some embodiments, R is —C₁₋₆ aliphatic substituted 1, 2, 3, 4, 5, or 6 times by —F. In some embodiments, R is —C₁₋₃ aliphatic substituted 1, 2, 3, 4, 5, or 6 times by —F. In some embodiments, R is —CH₃. In some embodiments, R is —CH₂CH₃. In some embodiments, R is —CF₃. In some embodiments, R is —CHF₂.

In some embodiments, R is optionally substituted 3-8 membered saturated or partially unsaturated monocyclic carbocyclyl. In some embodiments, R is unsubstituted 3, 4, 5, 6, 7, or 8 membered saturated or partially unsaturated monocyclic carbocyclyl. In some embodiments, R is 3, 4, 5, 6, 7, or 8 membered saturated or partially unsaturated monocyclic carbocyclyl substituted 1, 2, 3, 4, 5, or 6 times by -halogen, —CN, —NO₂, or —C₁₋₆ aliphatic, wherein the —C₁₋₆ aliphatic is optionally substituted 1, 2, 3, 4, 5, or 6 times by -halogen, —CN, or —NO₂. In some embodiments, R is 3, 4, 5, 6, 7, or 8 membered saturated or partially unsaturated monocyclic carbocyclyl substituted 1, 2, 3, 4, 5, or 6 times by -halogen. In some embodiments, R is 3, 4, 5, 6, 7, or 8 membered saturated or partially unsaturated monocyclic carbocyclyl substituted 1, 2, 3, 4, 5, or 6 times by —C₁₋₆ aliphatic, wherein the —C₁₋₆ aliphatic is optionally substituted 1, 2, 3, 4, 5, or 6 times by -halogen. In some embodiments, R is 3, 4, 5, 6, 7, or 8 membered saturated or partially unsaturated monocyclic carbocyclyl substituted 1, 2, 3, 4, 5, or 6 times by —F. In some embodiments, R is 3, 4, 5, 6, 7, or 8 membered saturated or partially unsaturated monocyclic carbocyclyl substituted 1, 2, 3, 4, 5, or 6 times by —C₁₋₆ aliphatic, wherein the —C₁₋₆ aliphatic is optionally substituted 1, 2, 3, 4, 5, or 6 times by —F.

In some embodiments, R is optionally substituted 3-8 membered saturated or partially unsaturated monocyclic heterocyclyl having 1-2 heteroatoms independently selected from nitrogen, oxygen, or sulfur. In some embodiments, R is unsubstituted 3, 4, 5, 6, 7, or 8 membered saturated or partially unsaturated monocyclic heterocyclyl having 1-2 heteroatoms independently selected from nitrogen, oxygen, or sulfur. In some embodiments, R is 3, 4, 5, 6, 7, or 8 membered saturated or partially unsaturated monocyclic heterocyclyl having 1-2 heteroatoms independently selected from nitrogen, oxygen, or sulfur, which is substituted 1, 2, 3, 4, 5, or 6 times by -halogen, —CN, —NO₂, or —C₁₋₆ aliphatic, wherein the —C₁₋₆ aliphatic is optionally substituted 1, 2, 3, 4, 5, or 6 times by -halogen, —CN, or —NO₂. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by -halogen. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by —C₁₋₆ aliphatic, wherein the —C₁₋₆ aliphatic is optionally substituted 1, 2, 3, 4, 5, or 6 times by -halogen. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by —F. In some embodiments, R is 3, 4, 5, 6, 7, or 8 membered saturated or partially unsaturated monocyclic heterocyclyl having 1-2 heteroatoms independently selected from nitrogen, oxygen, or sulfur substituted 1, 2, 3, 4, 5, or 6 times by —C₁₋₆ aliphatic, wherein the —C₁₋₆ aliphatic is optionally substituted 1, 2, 3, 4, 5, or 6 times by —F.

In some embodiments, R is selected from

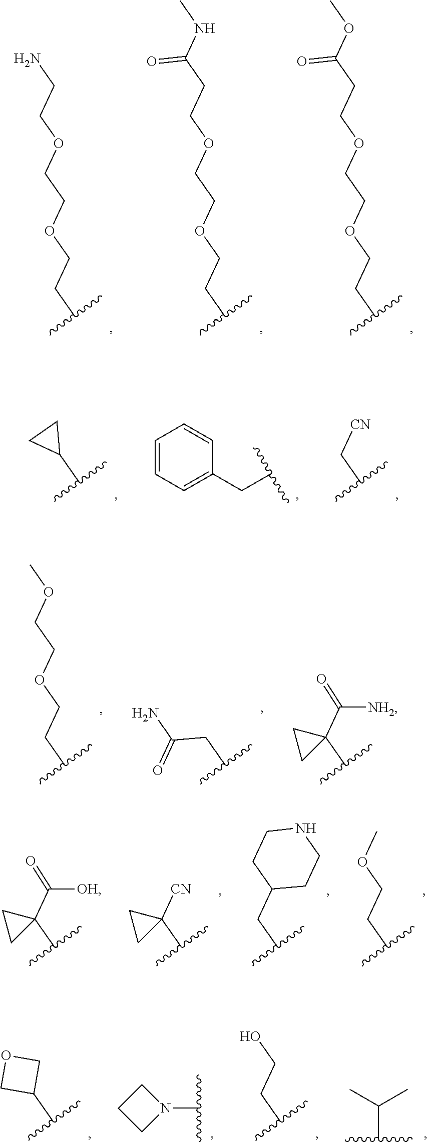

—CH₃, —CD₃, —CH₂CH₃, —CH₂C(O)NHCH₃,

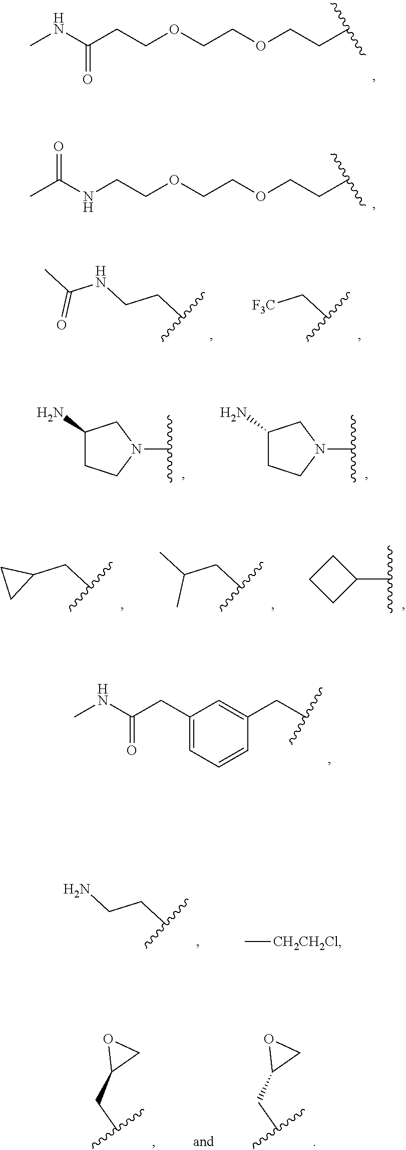

In some embodiments, R is selected from those depicted in Table E, below.

In some embodiments, a TEAD inhibitor is a compound selected from the following:

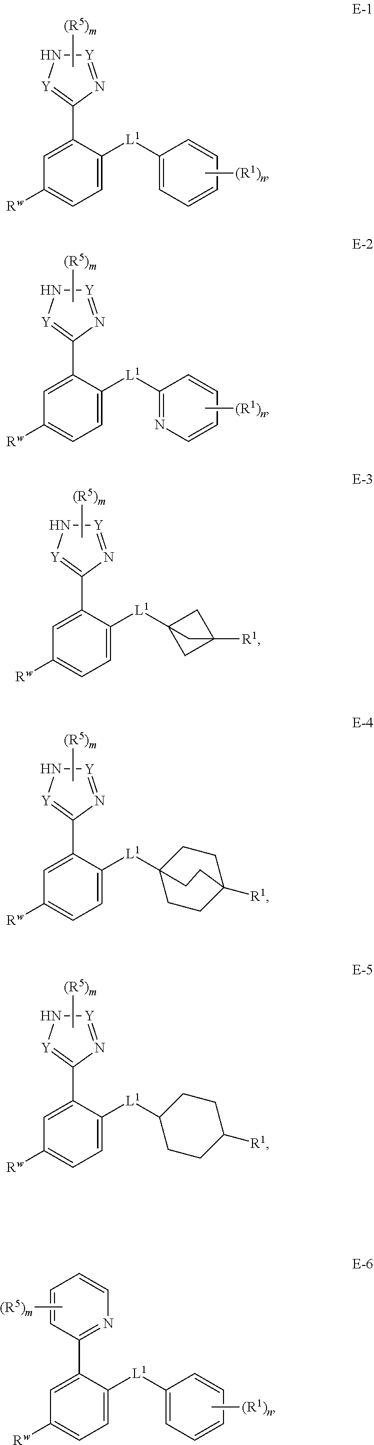

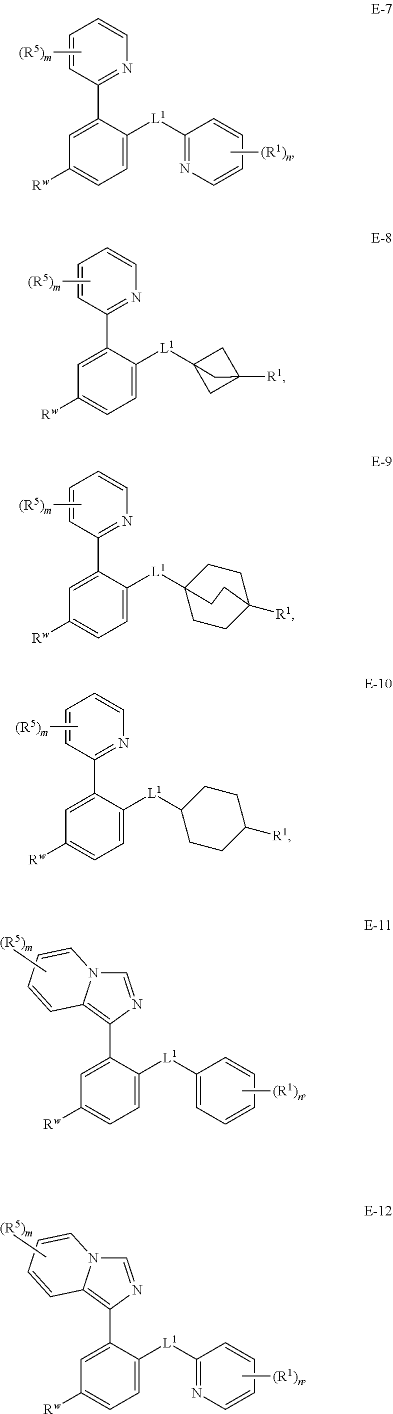

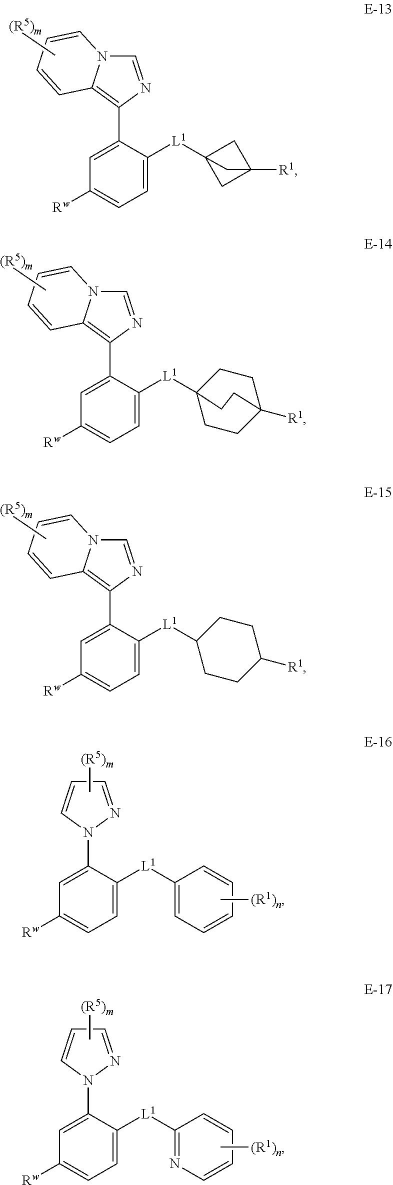

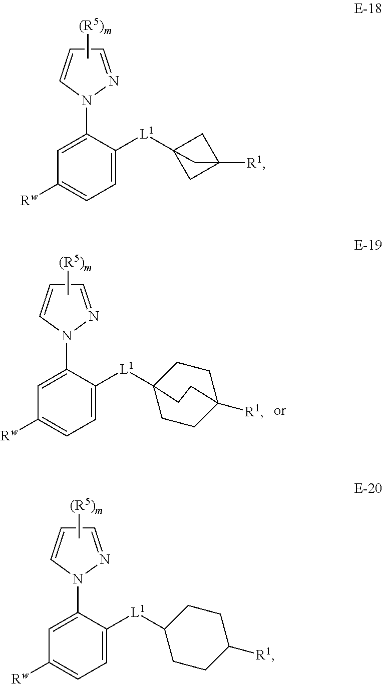

or a pharmaceutically acceptable salt thereof, wherein each of R¹, L¹, R^{w}, Y, m, n, and R⁵ is independently as defined and as described in embodiments in the section of TEAD Inhibitors of Formulae E, and E-1 to E-204.

In some embodiments, a TEAD inhibitor is a compound selected from the following:

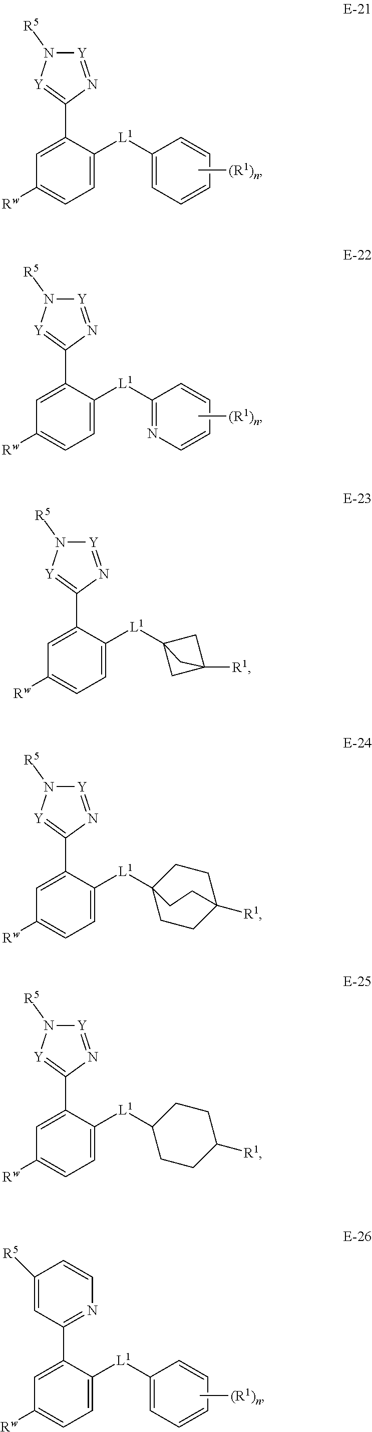

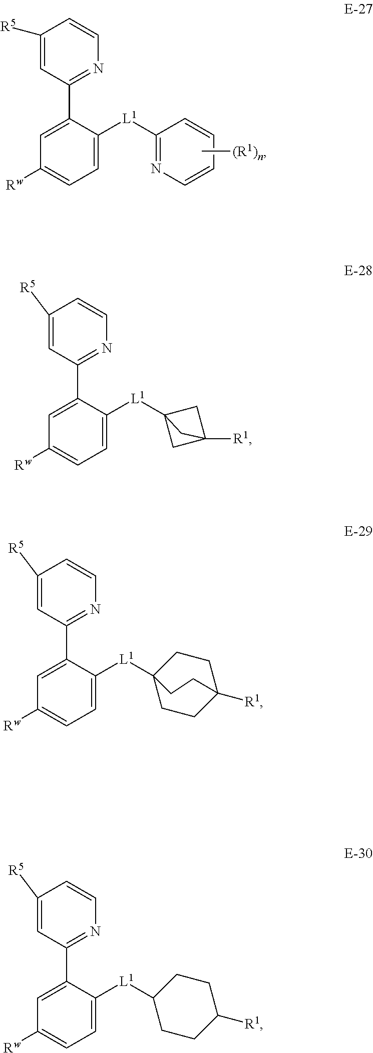

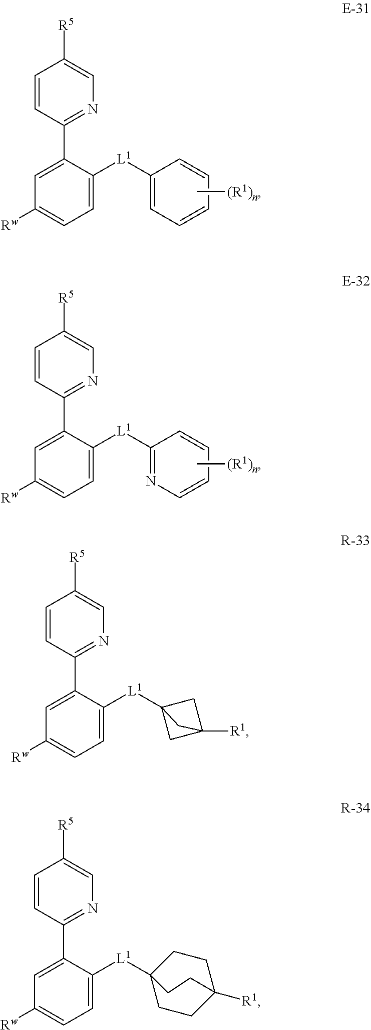

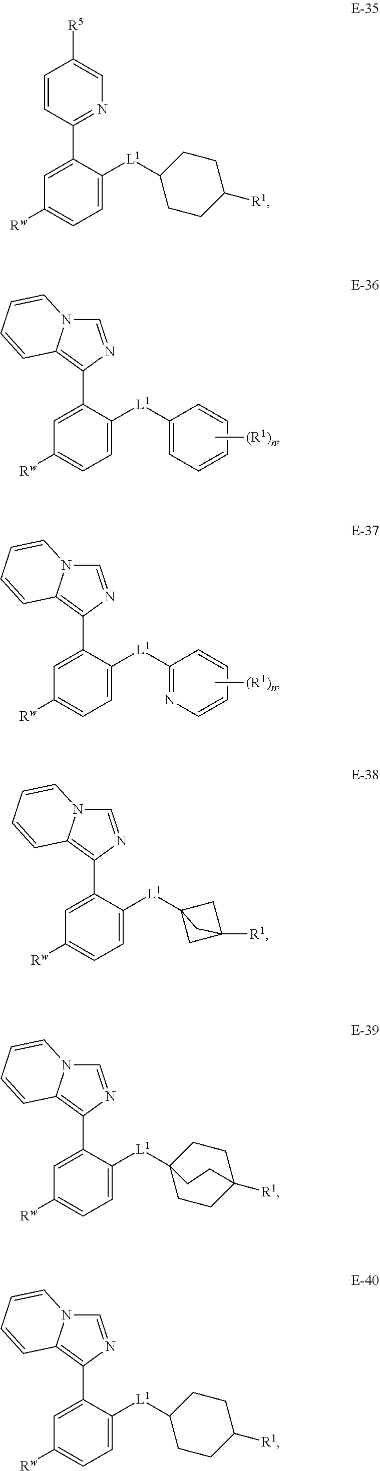

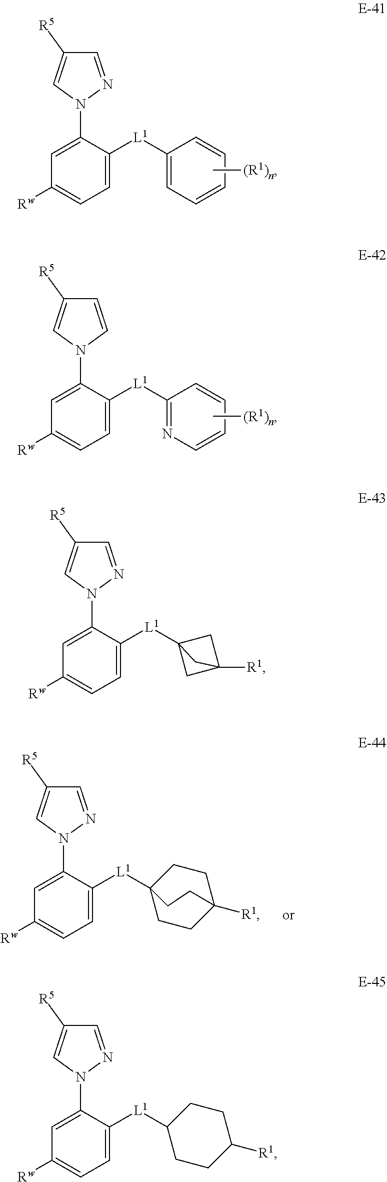

or a pharmaceutically acceptable salt thereof, wherein each of R¹, L¹, R^{w}, Y, n, and R⁵ is independently as defined above and as described in the section of TEAD Inhibitors of Formulae E, and E-1 to E-204.

In some embodiments, a TEAD inhibitor is a compound selected from the following:

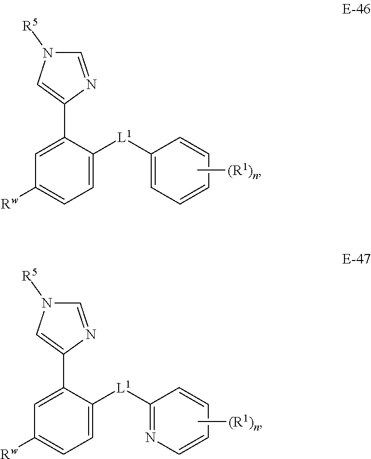

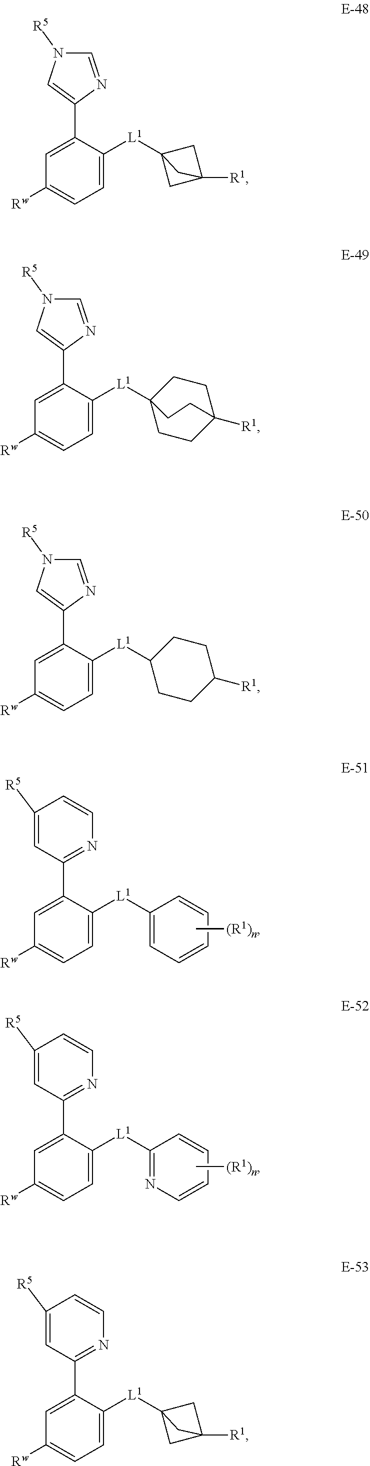

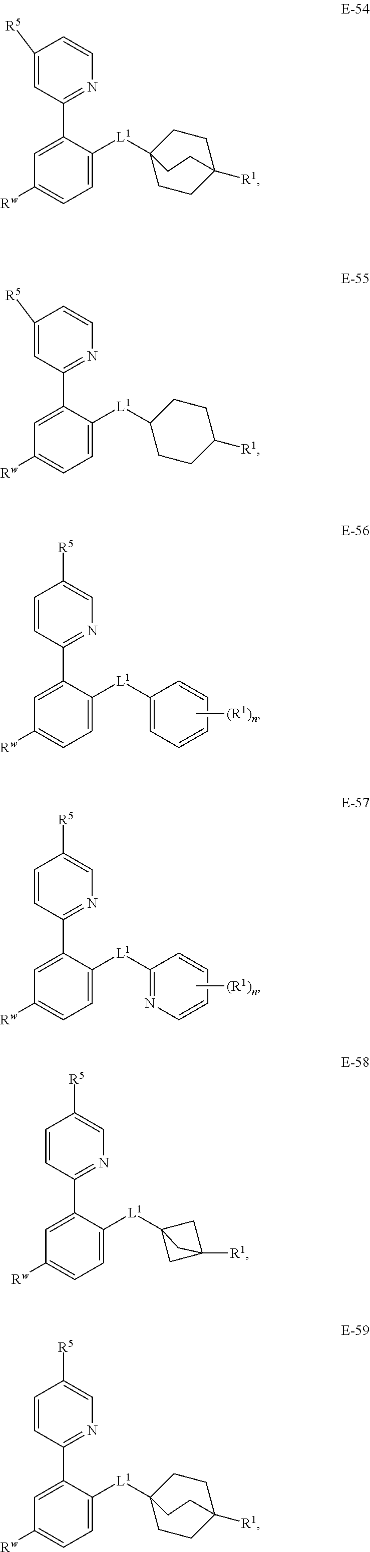

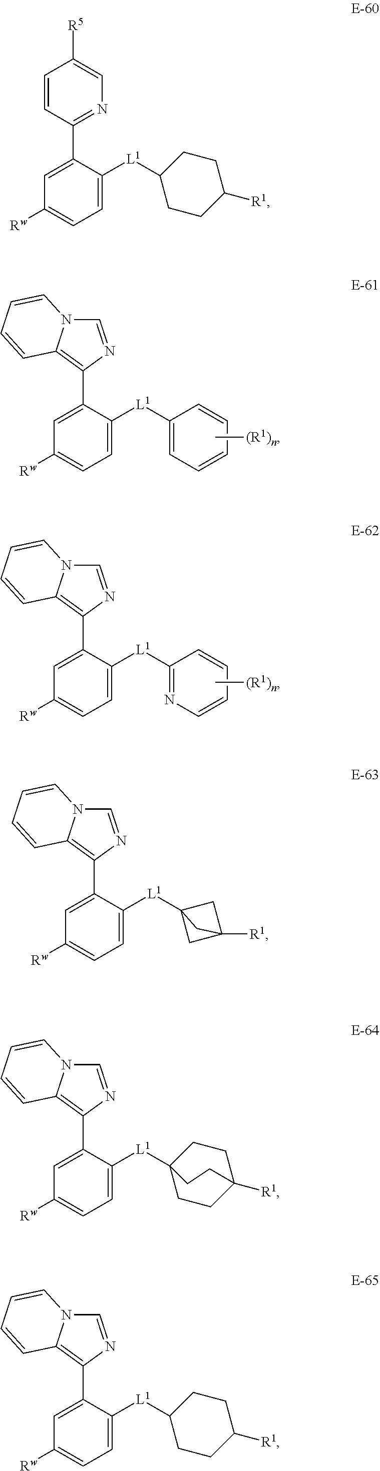

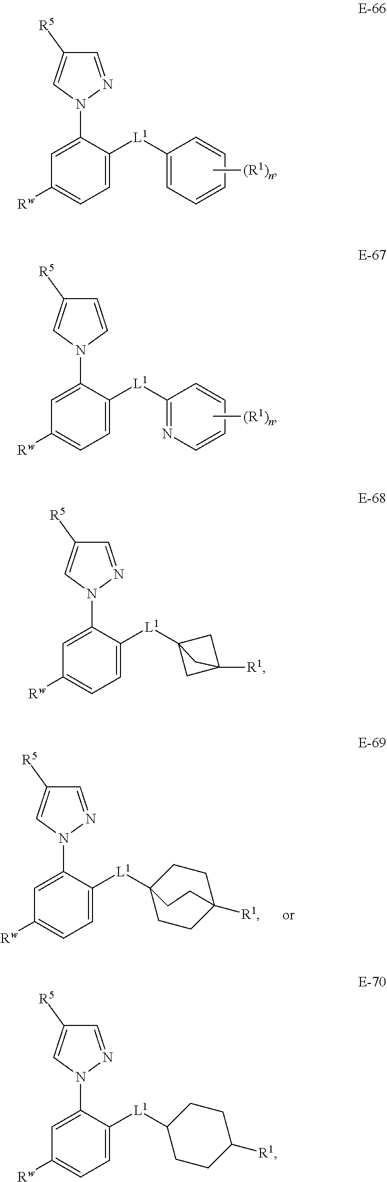

or a pharmaceutically acceptable salt thereof, wherein each of R¹, L¹, R^{w}, n, and R⁵ is independently as defined and as described in embodiments in the section of TEAD Inhibitors of Formulae E, and E-1 to E-204.

In some embodiments, a TEAD inhibitor is a compound selected from the following:

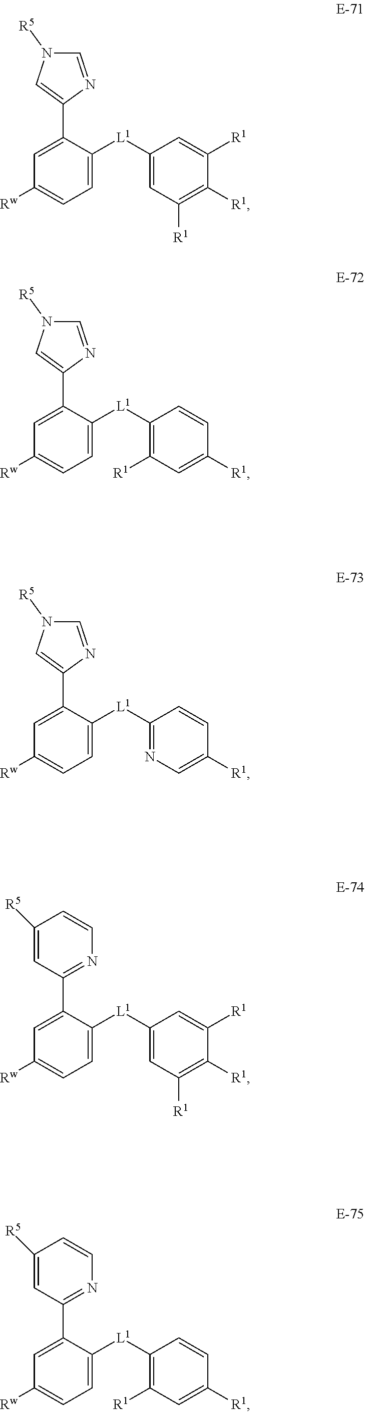

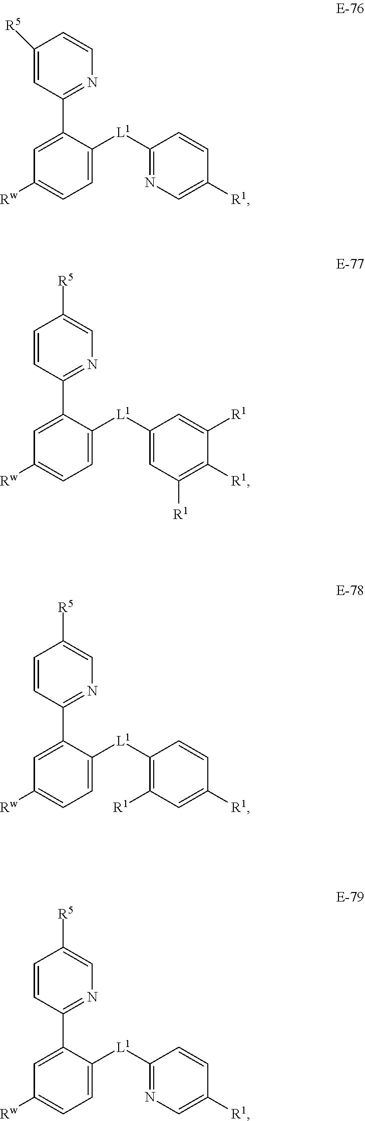

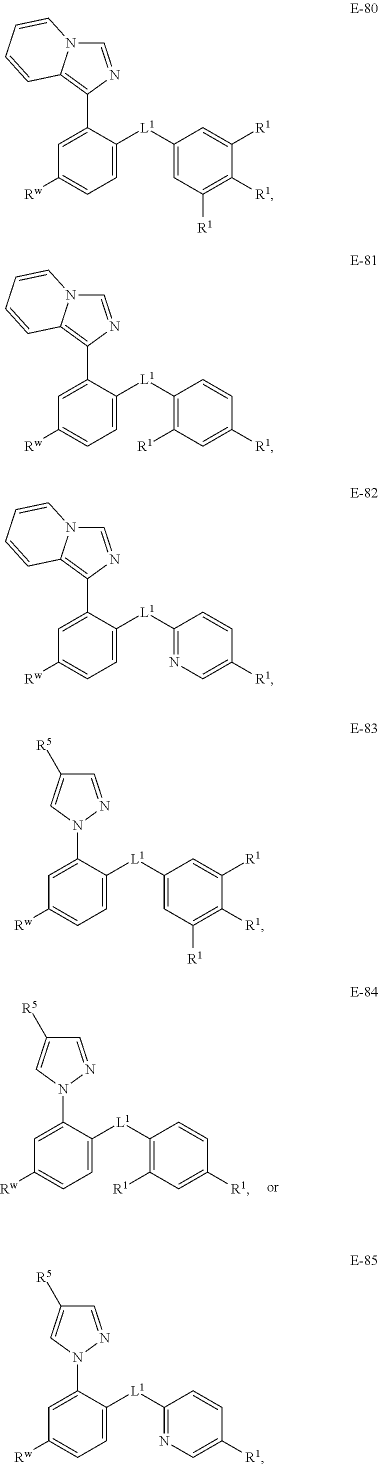

or a pharmaceutically acceptable salt thereof, wherein each of R¹, R^{w}, L¹, and R⁵ is independently as defined and as described in embodiments in the section of TEAD Inhibitors of Formulae E, and E-1 to E-204.

In some embodiments, a TEAD inhibitor is a compound selected from the following:

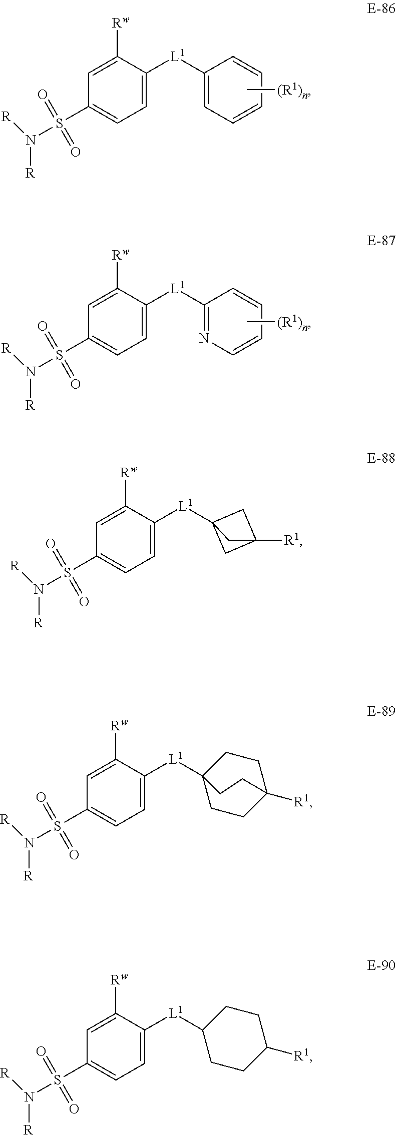

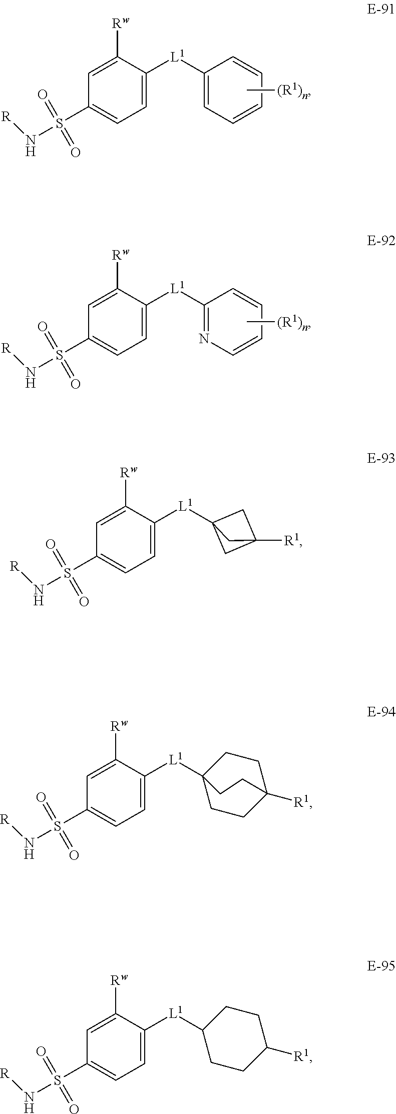

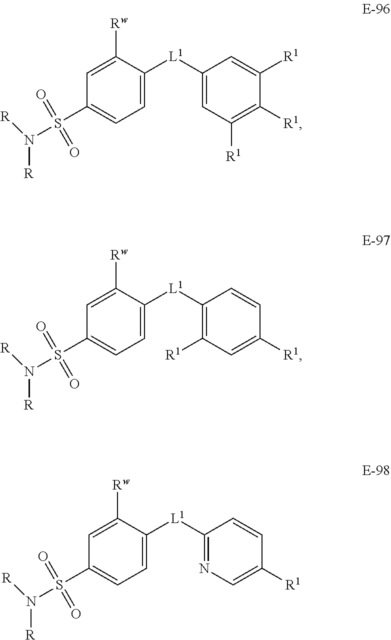

or a pharmaceutically acceptable salt thereof, wherein each of R, R¹, L¹, R^{w}, and n is independently as defined and as described in the section of TEAD Inhibitors of Formulae E, and E-1 to E-204.

In some embodiments, a TEAD inhibitor is a compound selected from the following:

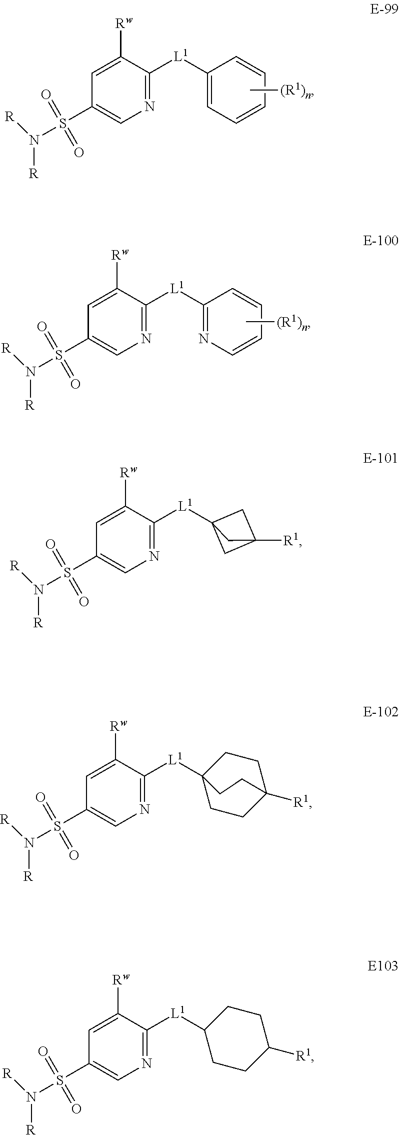

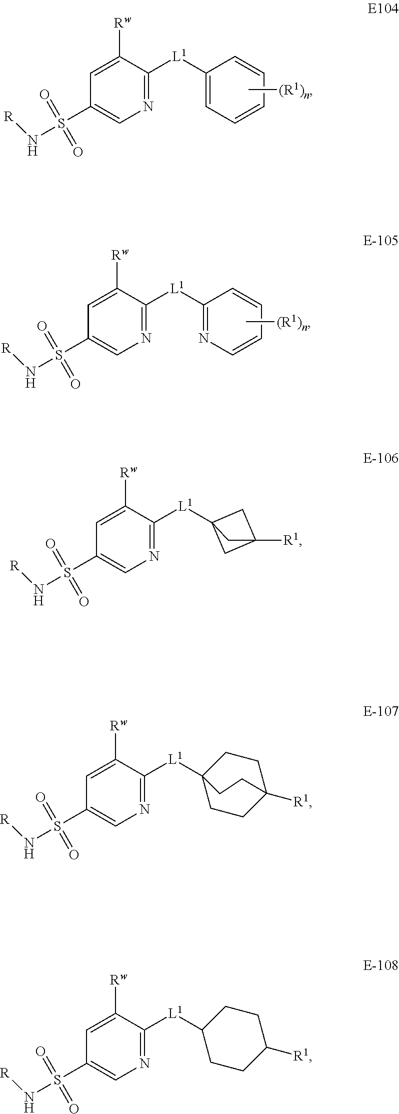

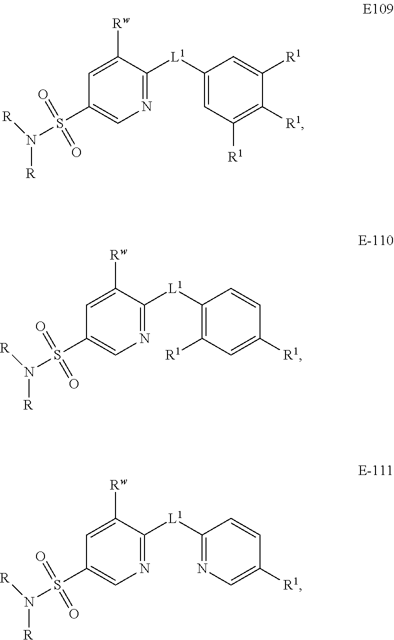

or a pharmaceutically acceptable salt thereof, wherein each of R, R¹, L¹, R^{w}, and n is independently as defined and as described in embodiments in the section of TEAD Inhibitors of Formulae E, and E-1 to E-204.

In some embodiments, a TEAD inhibitor is a compound selected from the following:

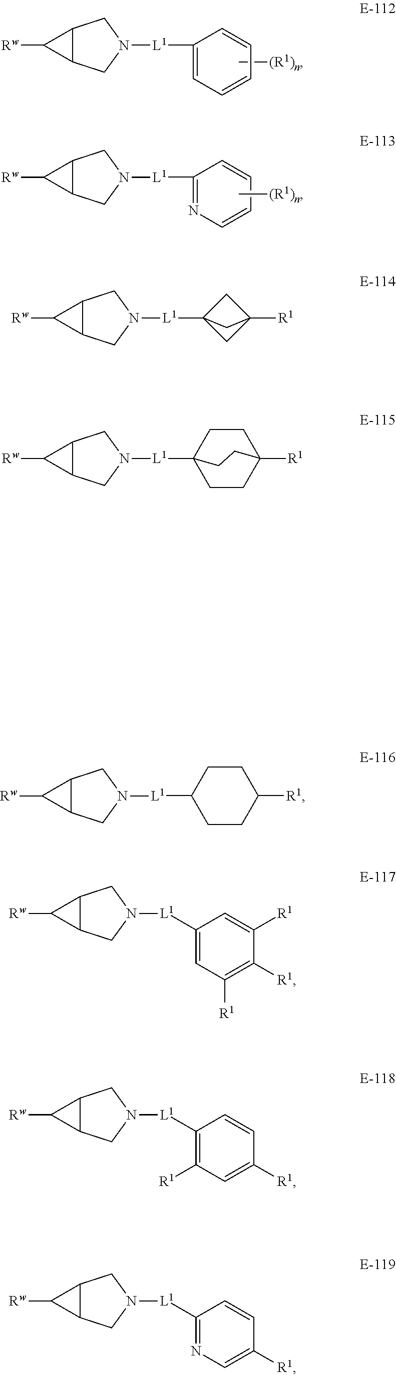

or a pharmaceutically acceptable salt thereof, wherein each of R¹, L¹, R^{w}, and n is independently as defined and as described in embodiments in the section of TEAD Inhibitors of Formulae E, and E-1 to E-204.

In some embodiments, a TEAD inhibitor is a compound selected from the following:

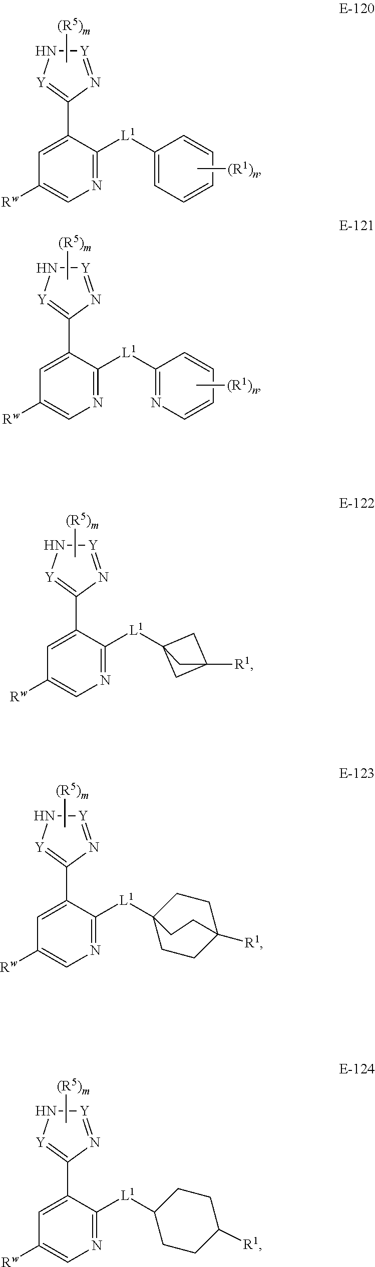

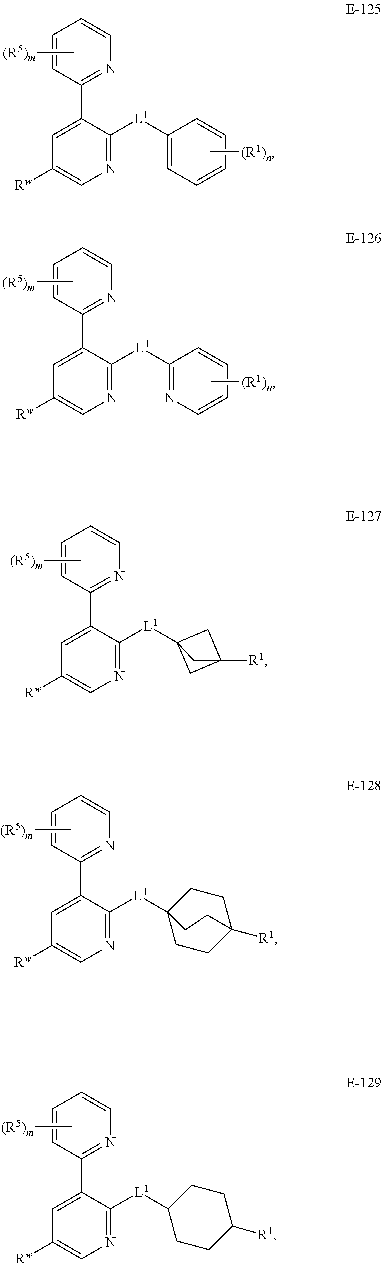

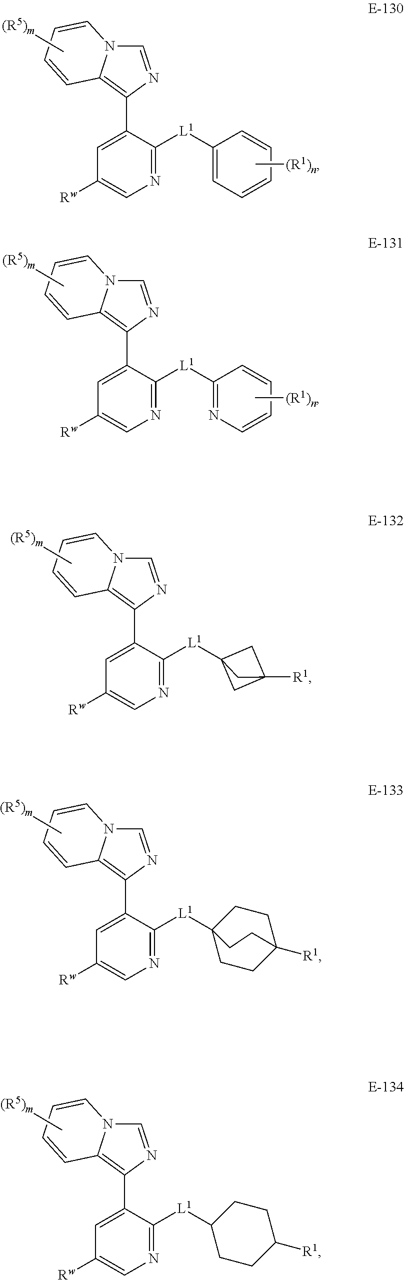

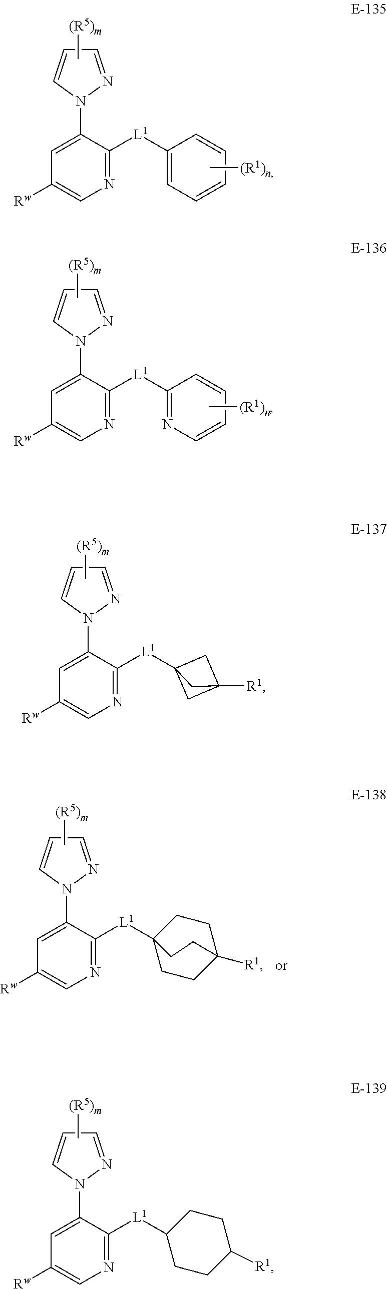

or a pharmaceutically acceptable salt thereof, wherein each of R¹, L¹, R^{w}, Y, m, n, and R⁵ is independently as defined and as described in embodiments in the section of TEAD Inhibitors of Formulae E, and E-1 to E-204.

In some embodiments, a TEAD inhibitor is a compound selected from the following:

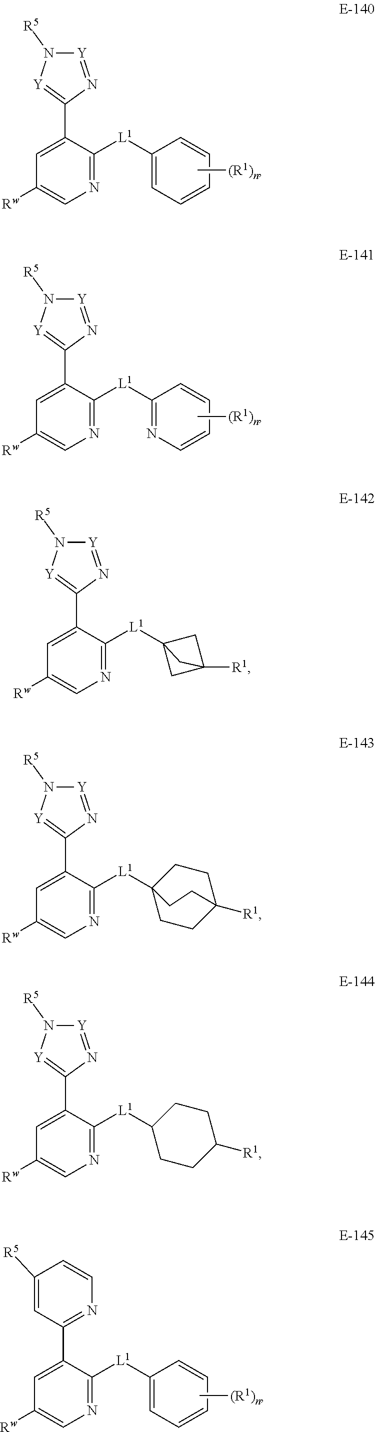

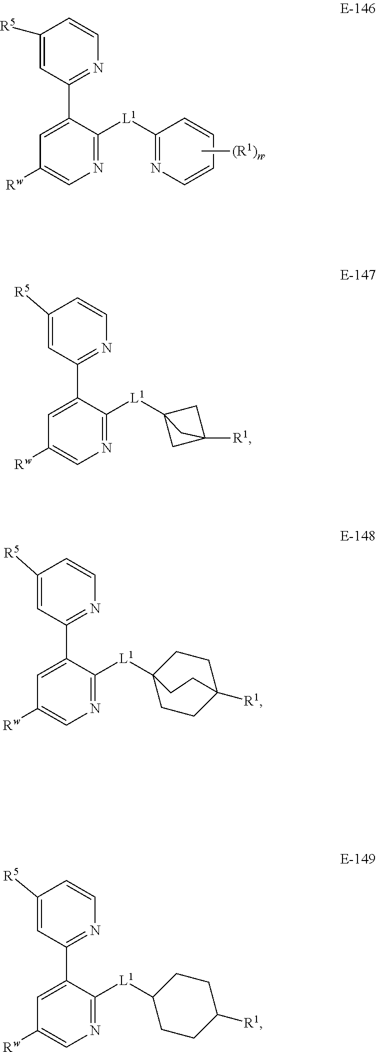

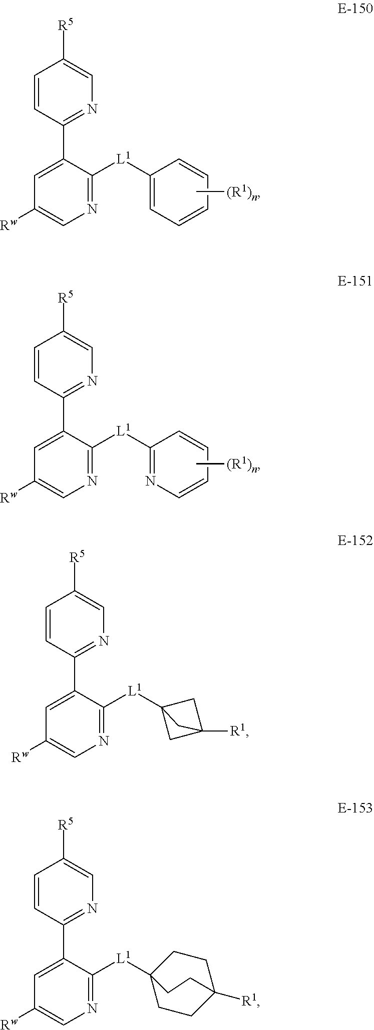

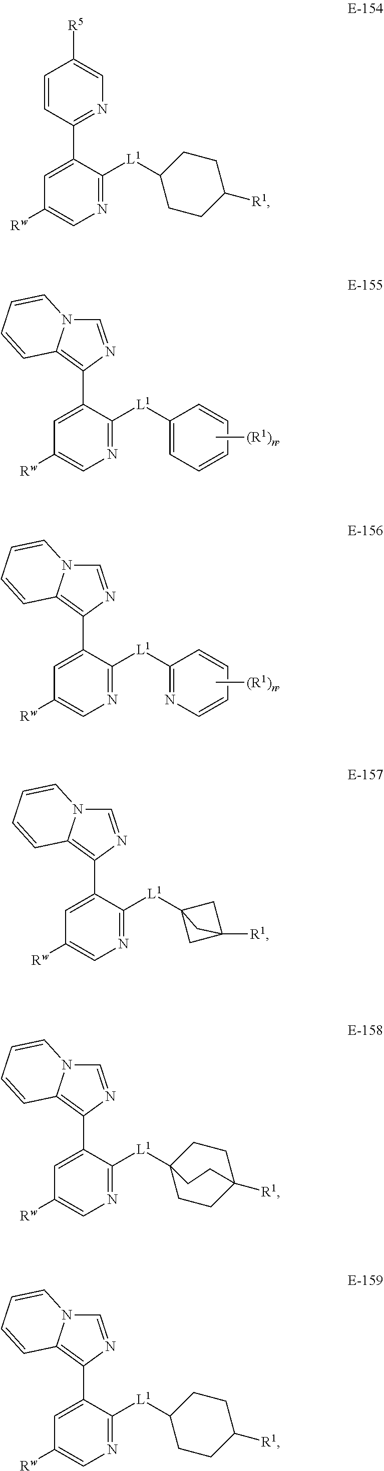

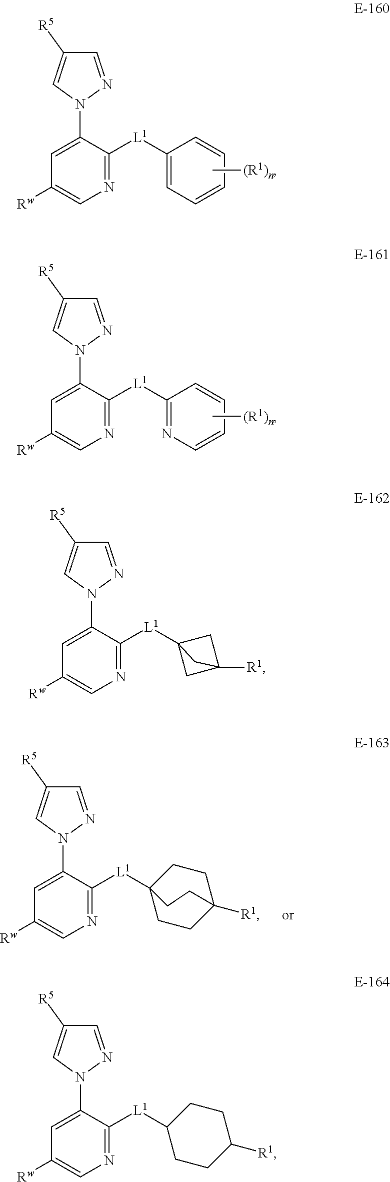

or a pharmaceutically acceptable salt thereof, wherein each of R¹, L¹, R^{w}, Y, n, and R⁵ is independently as defined and as described in embodiments in the section of TEAD Inhibitors of Formulae E, and E-1 to E-204.

In some embodiments, a TEAD inhibitor is a compound selected from the following:

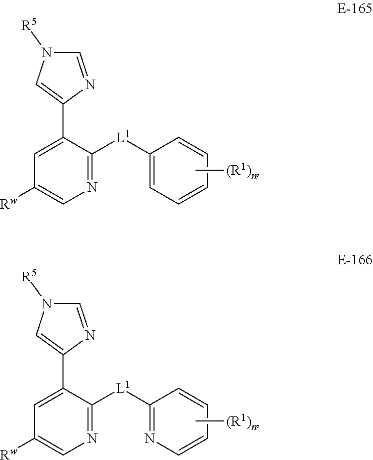

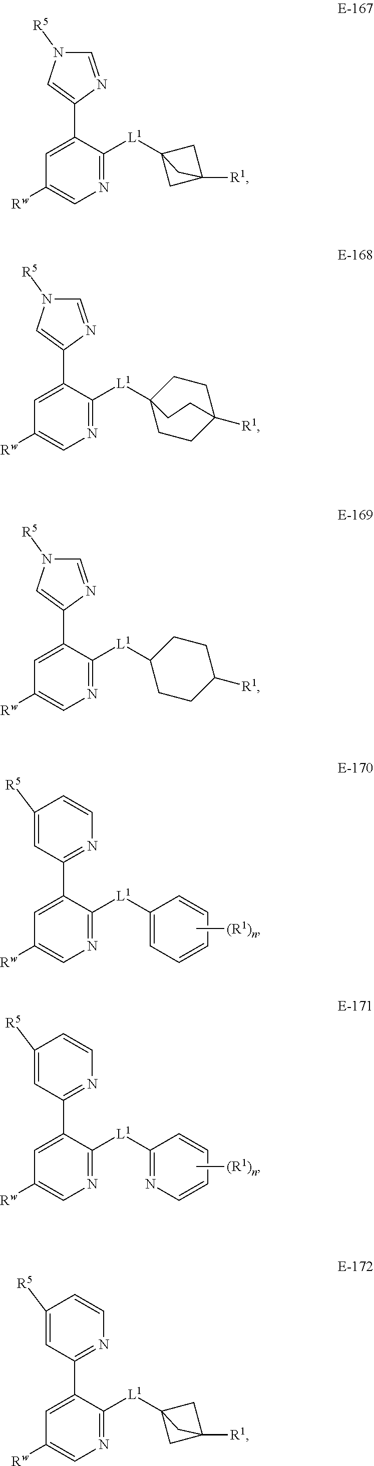

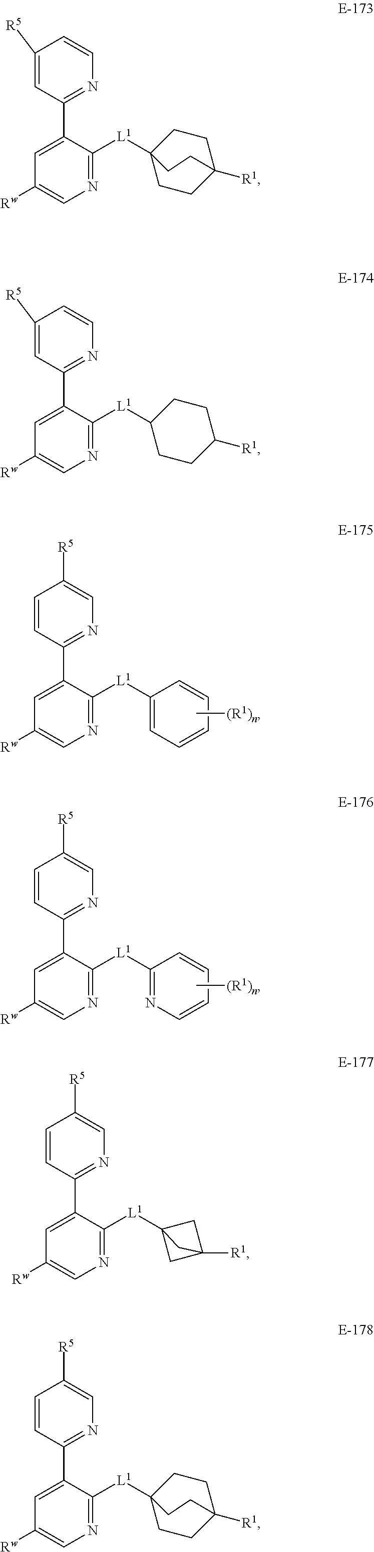

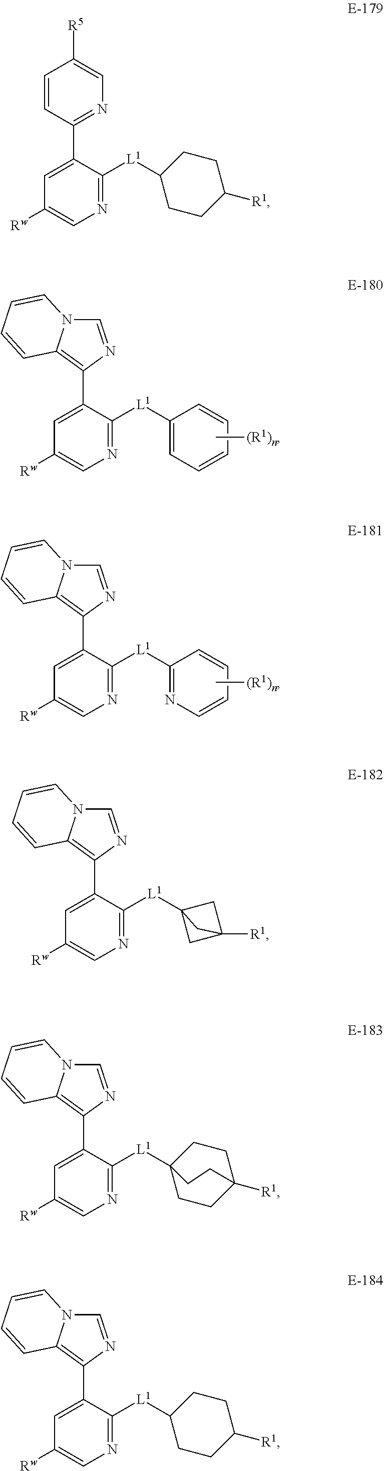

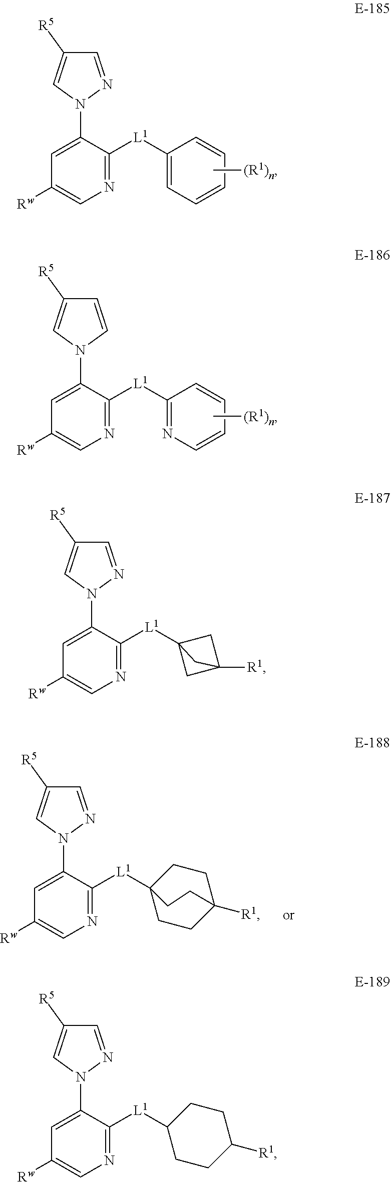

or a pharmaceutically acceptable salt thereof, wherein each of R¹, L¹, R^{w}, n, and R⁵ is independently as defined and as described in embodiments in the section of TEAD Inhibitors of Formulae E, and E-1 to E-204.

In some embodiments, a TEAD inhibitor is a compound selected from the following:

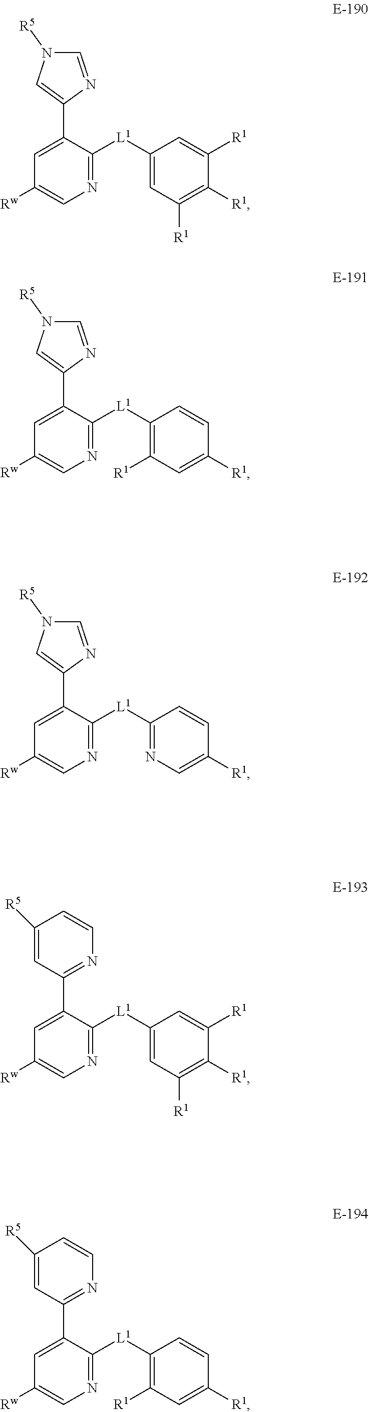

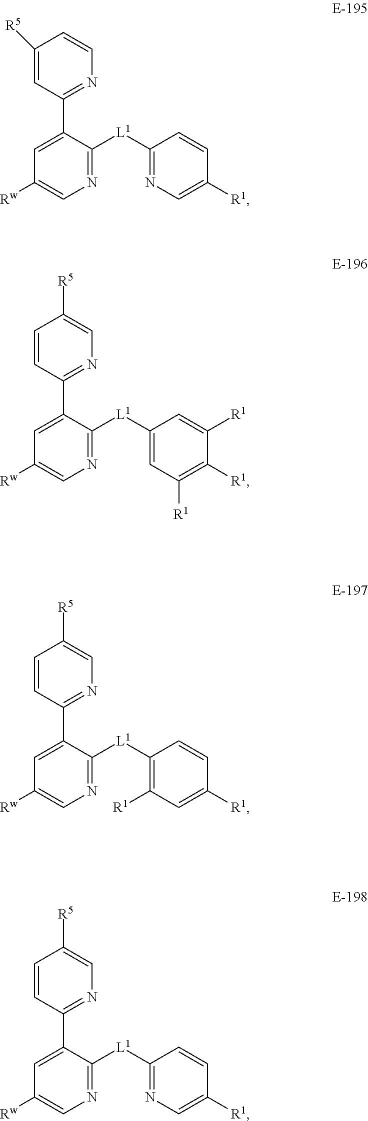

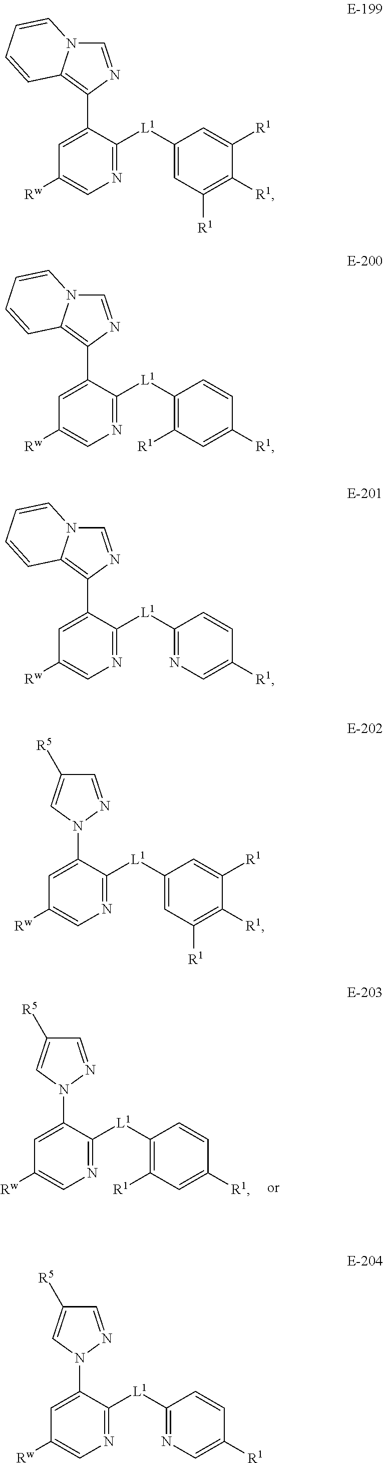

or a pharmaceutically acceptable salt thereof, wherein each of R¹, L¹, R^{w}, and R⁵ is independently as defined and as described in embodiments in the section of TEAD Inhibitors of Formulae E, and E-1 to E-204.

In some embodiments, a TEAD inhibitor is selected from those listed in Table E, or a pharmaceutically acceptable salt thereof.

TABLE E
Exemplified TEAD Inhibitors

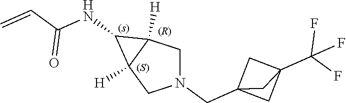

T-E-1

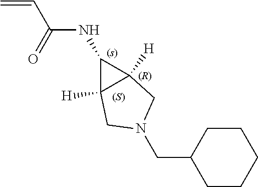

T-E-2

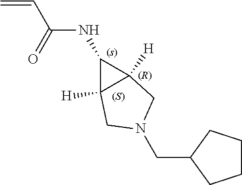

T-E-3

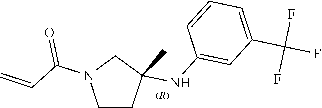

T-E-4

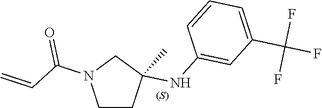

T-E-5

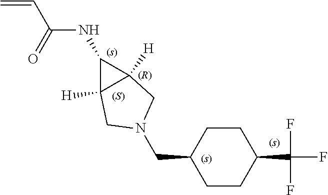

T-E-6

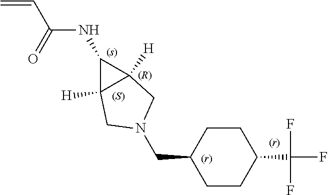

T-E-7

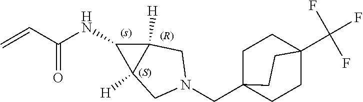

T-E-8

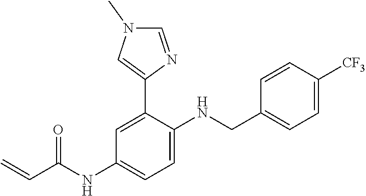 [SMILES: C=CC(=O)Nc1ccc(NCc2ccc(C(F)(F)F)cc2)c(-c2cn(C)cn2)c1]

T-E-9

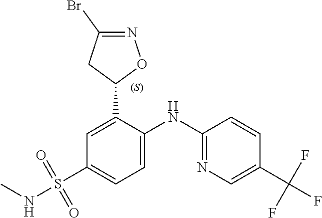

T-E-10

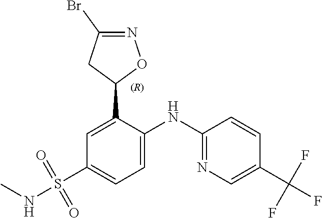

T-E-11

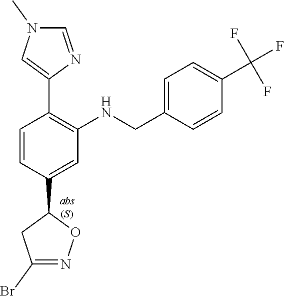

T-E-12

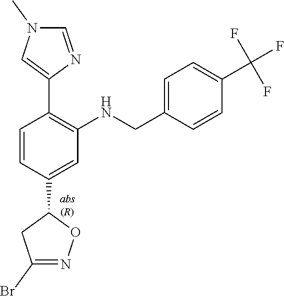

T-E-13

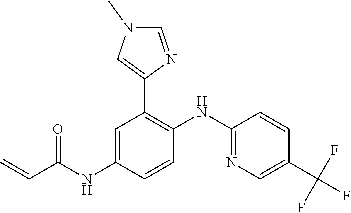 [SMILES: C=CC(=O)Nc1ccc(Nc2ccc(C(F)(F)F)cn2)c(-c2cn(C)cn2)c1]

T-E-14

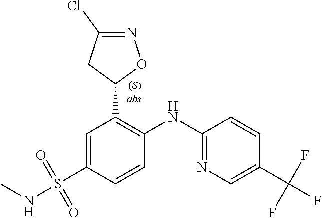

T-E-15

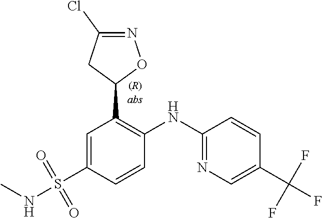

T-E-16

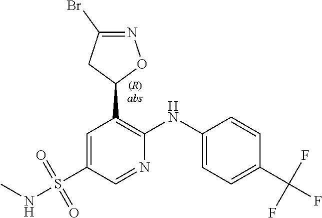

T-E-17

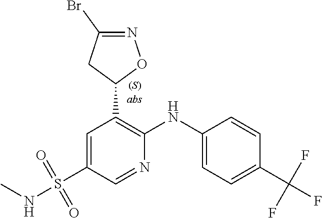

T-E-18

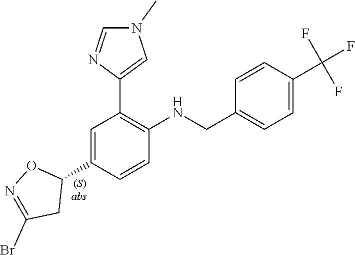

T-E-19

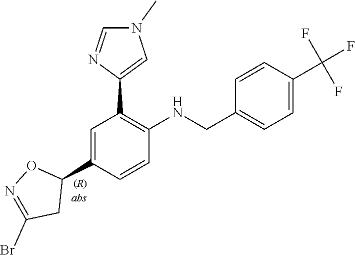

T-E-20

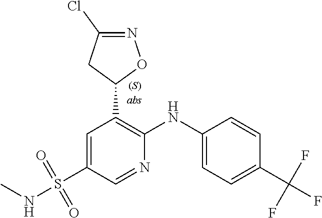

T-E-21

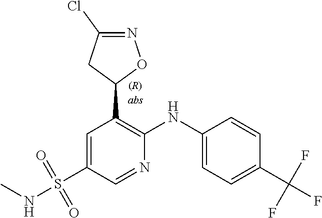

T-E-22

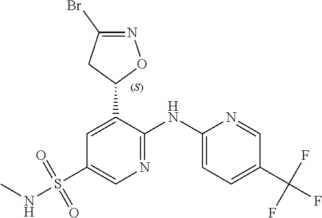

T-E-23

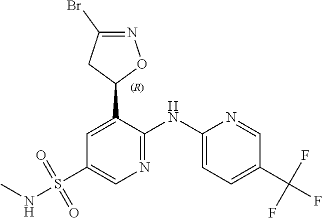

T-E-24

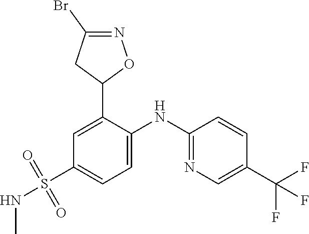 [SMILES: CNS(=O)(=O)c1ccc(Nc2ccc(C(F)(F)F)cn2)c(C2CC(Br)=NO2)c1]

T-E-25

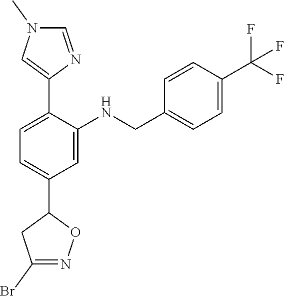 [SMILES: Cn1cnc(-c2ccc(C3CC(Br)=NO3)cc2NCc2ccc(C(F)(F)F)cc2)c1]

T-E-26

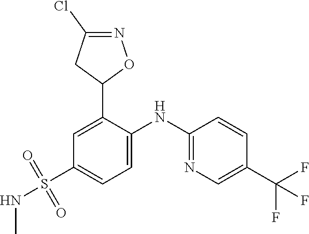 [SMILES: CNS(=O)(=O)c1ccc(Nc2ccc(C(F)(F)F)cn2)c(C2CC(Cl)=NO2)c1]

T-E-27

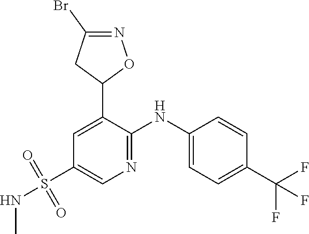 [SMILES: CNS(=O)(=O)c1cnc(Nc2ccc(C(F)(F)F)cc2)c(C2CC(Br)=NO2)c1]

T-E-28

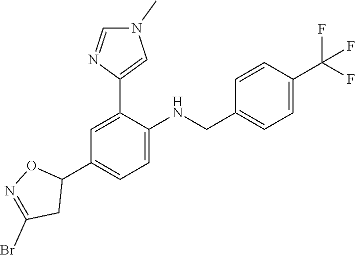 [SMILES: Cn1cnc(-c2cc(C3CC(Br)=NO3)ccc2NCc2ccc(C(F)(F)F)cc2)c1]

T-E-29

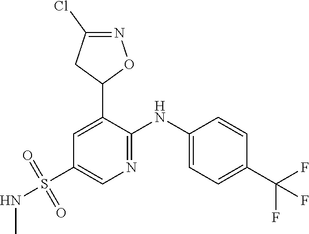 [SMILES: CNS(=O)(=O)c1cnc(Nc2ccc(C(F)(F)F)cc2)c(C2CC(Cl)=NO2)c1]

T-E-30

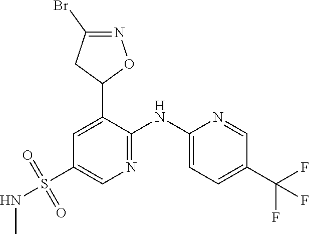 [SMILES: CNS(=O)(=O)c1cnc(Nc2ccc(C(F)(F)F)cn2)c(C2CC(Br)=NO2)c1]

T-E-31

As used herein, the terms “treatment,” “treat,” and “treating” refer to reversing, alleviating, delaying the onset of, or inhibiting the progress of a disease, or one or more symptoms thereof, as described herein. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example to prevent, or delay their recurrence.

As used herein, a patient or subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment or therapy.

A “therapeutically effective amount” or “therapeutically effective dosage” of a drug or therapeutic agent, such as a TEAD inhibitor, an EGFR inhibitor, and/or a MEK inhibitor, is any amount of the drug that, when used alone or in combination with another therapeutic agent, protects a patient or subject against the onset of a disease, such as cancer, or promotes disease regression evidenced by a decrease in severity of disease symptoms, an increase in frequency and duration of disease symptom-free periods, or a prevention of impairment or disability due to the disease affliction. The ability of a therapeutic agent, such as a TEAD inhibitor, an EGFR inhibitor, and/or a MEK inhibitor, to promote disease regression can be evaluated using a variety of methods known to the skilled practitioner, such as in human subjects during clinical trials, in animal model systems predictive of efficacy in humans, or by assaying the activity of the agent in in vitro assays.

In preferred embodiments, a therapeutically effective amount of the drug, such as a TEAD inhibitor, an EGFR inhibitor, and/or a MEK inhibitor, when used alone or in combination, promotes cancer regression to the point of eliminating the cancer. The term “promote(s) cancer regression” means that administering an effective amount of the drug, alone or in combination with one or more additional anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the terms “therapeutic benefit” or “benefit from therapy” refers to an improvement in one or more of overall survival, progression-free survival, partial response, complete response, and overall response rate and can also include a reduction in cancer or tumor growth or size, a decrease in severity of disease symptoms, an increase in frequency and duration of disease symptom-free periods, or a prevention of impairment or disability due to the disease affliction.

The terms “patient” or “subject” as used herein, means an animal, preferably a mammal, and most preferably a human.

An EGFR inhibitor can be administered separately from a TEAD inhibitor, as part of a multiple dosage regimen. Alternatively, an EGFR inhibitor may be part of a single dosage form, mixed together with an TEAD inhibitor in a single composition. If administered as a multiple dosage regime, an EGFR inhibitor and a TEAD inhibitor can be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, an EGFR inhibitor and a TEAD inhibitor are administered as a multiple dosage regimen with greater than 24 hours apart.

A MEK inhibitor can be administered separately from a TEAD inhibitor, as part of a multiple dosage regimen. Alternatively, a MEK inhibitor may be part of a single dosage form, mixed together with a TEAD inhibitor in a single composition. If administered as a multiple dosage regime, a MEK inhibitor and a TEAD inhibitor can be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, a MEK inhibitor and a TEAD inhibitor are administered as a multiple dosage regimen with greater than 24 hours apart.

A MEK inhibitor can be administered separately from a TEAD inhibitor and an EGFR inhibitor, as part of a multiple dosage regimen. Alternatively, a MEK inhibitor may be part of a single dosage form, mixed together with an TEAD inhibitor and an EGFR inhibitor in a single composition. If administered as a multiple dosage regime, a MEK inhibitor, an EGFR inhibitor, and a TEAD inhibitor can be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, a MEK inhibitor, an EGFR inhibitor, and a TEAD inhibitor are administered as a multiple dosage regimen with greater than 24 hours apart. In some embodiments, a TEAD inhibitor is N-methyl-3-(1-methyl-1H-imidazol-4-yl)-4-((4-(trifluoromethyl)benzyl)amino)benzenesulfonamide (Compound T-A-32), or a pharmaceutically acceptable salt thereof.

As used herein, the term “combination,” “combined,” and related terms refers to the simultaneous or sequential administration of therapeutic agents in accordance with this invention. For example, a TEAD inhibitor can be administered with an EGFR inhibitor simultaneously or sequentially in separate unit dosage forms or together in a single unit dosage form. Accordingly, the present invention provides a single unit dosage form comprising a TEAD inhibitor, an EGFR inhibitor, and a pharmaceutically acceptable carrier, adjuvant, or vehicle. A TEAD inhibitor can also be administered with a MEK inhibitor simultaneously or sequentially in separate unit dosage forms or together in a single unit dosage form. Accordingly, the present invention provides a single unit dosage form comprising a TEAD inhibitor and a MEK inhibitor, and a pharmaceutically acceptable carrier, adjuvant, or vehicle. A MEK inhibitor can also be administered with a TEAD inhibitor and an EGFR inhibitor simultaneously or sequentially in separate unit dosage forms or together in a single unit dosage form. Accordingly, the present invention provides a single unit dosage form comprising a TEAD inhibitor, an EGFR inhibitor, and a MEK inhibitor, and a pharmaceutically acceptable carrier, adjuvant, or vehicle.

### 4. Uses, Formulation and Administration

### Pharmaceutically Acceptable Compositions

In some embodiments, the present invention provides a pharmaceutical composition comprising a TEAD inhibitor, and a pharmaceutically acceptable carrier, adjuvant, or vehicle. In certain embodiments, the amount of a TEAD inhibitor in compositions of this invention is such that is effective to measurably inhibit TEAD, or a variant or mutant thereof, in a biological sample or in a patient. In some embodiments, a TEAD inhibitor is selected from those as described herein.

In certain embodiments, a composition of this invention is formulated for administration to a patient in need of such composition. In some embodiments, a composition of this invention is formulated for oral administration to a patient.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention can be administered orally, parenterally, by inhalation spray, topically, rectally, nasally, buccally, vaginally or via an implanted reservoir. “Administering,” as used herein, refers to the physical introduction of a composition comprising a therapeutic agent to a subject, using any of the various methods and delivery systems known to those skilled in the art. In some embodiments, a route of administration for a TEAD inhibitor is oral administration. In some embodiments, a route of administration for an EGFR inhibitor and/or an MEK inhibitor is intravenous, intramuscular, subcutaneous, intraperitoneal, spinal or other parenteral routes of administration, for example, by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

Sterile injectable forms of the compositions of this invention can be aqueous or oleaginous suspension. These suspensions can be formulated according to techniques known in the art using suitable dispersing or wetting agents and suspending agents. The sterile injectable preparation can also be a sterile injectable solution or suspension in a non-toxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium.

For this purpose, any bland fixed oil can be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

Pharmaceutically acceptable compositions of this invention can be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents can also be added.

Alternatively, pharmaceutically acceptable compositions of this invention can be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For topical applications, provided pharmaceutically acceptable compositions can be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions can be formulated in an ointment such as petrolatum.

Pharmaceutically acceptable compositions of this invention can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations can be administered with or without food. In some embodiments, pharmaceutically acceptable compositions of this invention are administered without food. In other embodiments, pharmaceutically acceptable compositions of this invention are administered with food.

The amount of a TEAD inhibitor that can be combined with the carrier materials to produce a composition in a single dosage form varies depending upon the host treated, the particular mode of administration. Preferably, provided compositions should be formulated so that a dosage of between 0.01-100 mg/kg body weight/day of a TEAD inhibitor can be administered to a patient receiving these compositions.

In those compositions comprising multiple therapeutic agents, the therapeutic agents can act synergistically. Therefore, the amount of each therapeutic agents in such compositions may be less than that required in a monotherapy utilizing only that therapeutic agent. In some embodiments, the amount of each therapeutic agent in the compositions comprising multiple therapeutic agents ranges from about 50% to 100% of the amount normally present in a composition comprising that agent as the only therapeutically active agent. In some embodiments, an EGFR inhibitor is administered at a dosage of about 50%, about 55%, about 60%, about 65%, about 70%, about 75%, about 80%, about 85%, about 90%, or about 95% of the amount normally administered for that agent. In some embodiments, a MEK inhibitor is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approved for dosing per the FDA label insert.

It should also be understood that a specific dosage and treatment regimen for any particular patient depends upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition also depends upon the particular compound in the composition.

### Uses of Compounds and Pharmaceutically Acceptable Compositions

In some embodiments, the present invention provides a method for treating cancer in a patient comprising administering to the patient a therapeutically effective amount of a TEAD inhibitor, or a pharmaceutical composition thereof, and an EGFR inhibitor. In some embodiments, the present invention provides a method for treating cancer in a patient comprising administering to the patient a therapeutically effective amount of a TEAD inhibitor, or a pharmaceutical composition thereof, and a MEK inhibitor. In some embodiments, the present invention provides a method for treating cancer in a patient comprising administering to the patient a therapeutically effective amount of a TEAD inhibitor, or a pharmaceutical composition thereof, an EGFR inhibitor, and an MEK inhibitor.

### Cancer

A “cancer,” as used herein, refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

A cancer to be treated in the present invention includes, but is not limited to, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.

In some embodiments of the methods and uses described herein, a cancer is mediated by activation of transcriptional coactivator with PDZ binding motif/Yes-associated protein transcription coactivator (TAZ/YAP). In some embodiments of the methods and uses described herein, a cancer is mediated by modulation of the interaction of YAP/TAZ with TEAD (e.g., TEAD1, TEAD2, TEAD3, and/or TEAD4). In some embodiments of the methods and uses described herein, the cancer is characterized by or associated with increased TEAD (e.g., TEAD1, TEAD2, TEAD3, and/or TEAD4) expression and/or increased TEAD (e.g., TEAD1, TEAD2, TEAD3, and/or TEAD4) activity. In some embodiments of the methods and uses described herein, the cancer is a cancer in which YAP is localized in the nucleus of the cancer cells.

In some embodiments of the methods and uses described herein, the cancer is characterized or associated with a genetic alteration in one or more Hippo pathway genes. As used herein, the term “genetic alteration in one or more Hippo pathway genes” refers to that certain percentage of cells in a sample, such as a tumor sample, having a detectable amount of genetic alteration in one or more Hippo pathway genes. As used herein, a genetic alteration in a gene, such as a Hippo pathway gene, can refer, for example, to a loss-of-function mutation in the gene (including, for example, frameshifts, nonsense mutations and splicing mutations), a change in gene copy number (including, for example, copy gain, amplification, copy loss, or deletion), or a fusion of the gene with another gene, such as, for example, a TAZ-CAMTA1 fusion or YAP1-TFE3 fusion. In some embodiments, genetic alteration in Hippo pathway genes refers to that about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or 100% of cells, such as tumor cells, in a sample have at least about three copies of genetically altered Hippo pathway genes, at least about four copies of genetically altered Hippo pathway genes, at least about five copies of genetically altered Hippo pathway genes, at least about six copies of genetically altered Hippo pathway genes, at least about seven copies of genetically altered Hippo pathway genes, at least about eight copies of genetically altered Hippo pathway genes, at least about nine copies of genetically altered Hippo pathway genes, at least about ten copies of genetically altered Hippo pathway genes, at least about eleven copies of genetically altered Hippo pathway genes, at least about twelve copies of genetically altered Hippo pathway genes, at least about nine copies of genetically altered Hippo pathway genes, at least about ten copies of genetically altered Hippo pathway genes, at least about eleven copies of genetically altered Hippo pathway genes, at least about twelve copies of genetically altered Hippo pathway genes, at least about thirteen copies of genetically altered Hippo pathway genes, at least about fourteen copies of genetically altered Hippo pathway genes, at least about fifteen copies of genetically altered Hippo pathway genes, at least about twenty copies of genetically altered Hippo pathway genes, or more. In some embodiments, genetic alteration in Hippo pathway genes refers to that about 10% tumor cells in a sample have at least about 15 copies of genetically altered Hippo pathway genes. In some embodiments, genetic alteration in Hippo pathway genes refers to that about 40% tumor cells in a sample have at least about 4 copies of genetically altered Hippo pathway genes. In some embodiments, genetic alteration in Hippo pathway genes refers to that about 10% tumor cells in a sample have at least about four copies of genetically altered Hippo pathway genes.

In some embodiments, a Hippo pathway gene is NF2. In some embodiments, the genetic alteration in the one or more Hippo pathway genes is NF2 deficiency. In some embodiments, NF2 deficiency refers to NF2 loss of function mutations. In some embodiments, NF2 deficiency refers to NF2 copy losses or deletions. In some embodiments, NF2 deficiency refers to absent or very low NF2 mRNA expression.

In some embodiments, a Hippo pathway gene is YAP1. In some embodiments, the genetic alteration in the one or more Hippo pathway genes is YAP1 amplification. In some embodiments, the genetic alteration in the one or more Hippo pathway genes is a YAP1 fusion, such as a YAP1-TFE3 fusion. In some embodiments, a Hippo pathway gene is TAZ. In some embodiments, the genetic alteration in the one or more Hippo pathway genes is TAZ amplification. In some embodiments, the genetic alteration in the one or more Hippo pathway genes is a TAZ fusion, such as a TAZ-CAMTA1 fusion. In some embodiments, a Hippo pathway gene is LATS 1/2. In some embodiments, the genetic alteration in the one or more Hippo pathway genes is LATS 1/2 copy number loss or deletion. In some embodiments, a Hippo pathway gene is MST1/2. In some embodiments, a Hippo pathway gene is BAP1.

In some embodiments, a cancer is characterized by a mutant Gα-protein. In some embodiments, a mutant Gα-protein is selected from G12, G13, Gq, G11, Gi, Go, and Gs. In some embodiments, a mutant Gα-protein is G12. In some embodiments, a mutant Gα-protein is G13. In some embodiments, a mutant Gα-protein is Gq. In some embodiments, a mutant Gα-protein is G11. In some embodiments, a mutant Gα-protein is Gi. In some embodiments, a mutant Gα-protein is Go. In some embodiments, a mutant Gα-protein is Gs.

In some embodiments, the cancer is lung cancer, thyroid cancer, ovarian cancer, colorectal cancer, prostate cancer, cancer of the pancreas, cancer of the esophagus, liver cancer, breast cancer, skin cancer, mesothelioma, or epithelioid hemangioendothelioma, or EHE. In some embodiments, the cancer is mesothelioma, such as malignant mesothelioma. In some embodiments, the cancer is EHE. In some embodiments, the lung cancer is non-small cell lung cancer (NSCLC).

In some embodiments, the cancer is a solid tumor. In some embodiments, the cancer is a locally advanced or metastatic solid tumor.

In some embodiments, the cancer is a KRAS mutant cancer. In some embodiments, the KRAS mutant cancer harbors the KRAS G12C mutation. In some embodiments, the KRAS mutant cancer harbors the KRAS G12D mutation. In some embodiments, the KRAS mutant cancer harbors the KRAS G12V mutation. In some embodiments, the KRAS mutant cancer harbors the KRAS G13 mutation. In some embodiments, the KRAS mutant cancer harbors one or more KRAS mutations selected from a KRAS G12C, a KRAS G12D mutation, a KRAS G12V mutation, and a KRAS G13 mutation. In some embodiments, the cancer is a KRAS mutant lung cancer.

Cancer includes, in some embodiments, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the cancer is Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Cancer, Esophageal Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenstrom Macroglobulinemia, or Wilms Tumor.

In certain embodiments, the cancer is selected from bladder cancer, breast cancer (including TNBC), cervical cancer, colorectal cancer, chronic lymphocytic leukemia (CLL), diffuse large B-cell lymphoma (DLBCL), esophageal adenocarcinoma, glioblastoma, head and neck cancer, leukemia (acute and chronic), low-grade glioma, lung cancer (including adenocarcinoma, non-small cell lung cancer, and squamous cell carcinoma), Hodgkin's lymphoma, non-Hodgkin lymphoma (NHL), melanoma, multiple myeloma (MM), ovarian cancer, pancreatic cancer, prostate cancer, renal cancer (including renal clear cell carcinoma and kidney papillary cell carcinoma), and stomach cancer.

In some embodiments, the cancer is small cell lung cancer, non-small cell lung cancer, colorectal cancer, multiple myeloma, acute myeloid leukemia (AML), acute lymphoblastic leukemia (ALL), pancreatic cancer, liver cancer, hepatocellular cancer, neuroblastoma, other solid tumors or other hematological cancers.

In some embodiments, the cancer is small cell lung cancer, non-small cell lung cancer, colorectal cancer, multiple myeloma, or AML.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, the methods or uses described herein inhibit or reduce or arrest or ameliorate the growth or spread of a cancer or tumor. In some embodiments, the tumor is treated by arresting, reducing, or inhibiting further growth of the cancer or tumor. In some embodiments, the methods or uses described herein increase or potentiate or activate one or more immune responses to inhibit or reduce or arrest or ameliorate the growth or spread of a cancer or tumor. In some embodiments, the cancer or tumor is treated by reducing the size (e.g., volume or mass) of the cancer or tumor by at least 5%, at least 10%, at least 25%, at least 50%, at least 75%, at least 90%, at least 95%, at least 96%, at least 97%, at least 98%, or at least 99% relative to the size of the cancer or tumor prior to treatment. In some embodiments, cancers or tumors are treated by reducing the quantity of the cancers or tumors in the patient by at least 5%, at least 10%, at least 25%, at least 50%, at least 75%, at least 90%, at least 95%, at least 96%, at least 97%, at least 98%, or at least 99% relative to the quantity of cancers or tumors prior to treatment.

In some embodiments, a patient treated using the methods or uses described herein exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the treatment is initiated. In some embodiments, a patient treated using the methods or uses described herein exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about 14 months, at least about 16 months, at least about 18 months, at least about 20 months, at least about 22 months, at least about two years, at least about three years, at least about four years, or at least about five years after the treatment is initiated.

In some embodiments, a patient treated using the methods or uses described herein exhibits an objective response rate (ORR) of at least about 15%, at least about 20%, at least about 25%, at least about 30%, about 35%, about 40%, about 45%, about 50%, about 55%, about 60%, about 65%, about 70%, about 75%, about 80%, about 85%, about 90%, about 95%, or about 100%.

The compounds and compositions as described herein, can be administered using any amount and any route of administration effective for treating or lessening the severity of a cancer. The exact amount required varies from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention is decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The terms “patient” or “subject,” as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, a TEAD inhibitor can be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type can also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The following examples are provided for illustrative purposes only and are not to be construed as limiting this invention in any manner.

### EXEMPLIFICATION

The TEAD inhibitors described herein can be produced by organic synthesis methods known to one of ordinary skill in the art. Additionally, certain TEAD inhibitors can be prepared as described in Pobbati et al., “Targeting the Central Pocket in Human Transcription Factor TEAD as a Potential Cancer Therapeutic Strategy,” Structure 2015, 23, 2076-2086; Gibault et al., “Targeting Transcriptional Enhanced Associate Domains (TEADs),” J. Med. Chem. 2018, 61, 5057-5072; Bum-Erdene et al., “Small-Molecule Covalent Modification of Conserved Cysteine Leads to Allosteric Inhibition of the TEAD. Yap Protein-Protein Interaction,” Cell Chemical Biology 2019, 26, 1-12; Holden et al., “Small Molecule Dysregulation of TEAD Lipidation Induces a Dominant-Negative Inhibition of HippoPathway Signaling,” Cell Reports 2020, 31, 107809; and the following international and US patent publications, WO 2017/053706, WO 2017/111076, WO 2018/204532, WO 2018/235926, US 20190010136, WO 2019/040380, WO 2019/113236, WO 2019/222431, WO 2019/232216, WO 2020/051099, WO 2020/081572, WO 2020/097389, WO 2020/190774, WO 2020/214734, WO 2020/243415, and WO 2020/243423, the contents of each of which are herein incorporated by reference in their entireties.

### Example 1. The Apoptosis Induction Effects of a TEAD Inhibitor T-A-32, an EGFR Inhibitor Osimertinib, an MEK Inhibitor Trametinib, and Combinations Thereof in HCC4006 and HCC827 Cell Lines

This study determined the ability of T-A-32 to enhance apoptosis in combination with osimertinib and trametinib in epidermal growth factor receptor (EGFR) mutant cell lines, and in combination with trametinib in Kirsten rat sarcoma viral oncogene (KRAS) or v raf murine sarcoma viral oncogene homolog B (BRAF) mutant cancer cell lines.

Three EGFR mutant cell lines (HCC4006, HCC827, and NCI-H1975), 5 KRAS mutant cell lines (A549, HCT116, Capan-2, Calu-1, and LoVo), and 1 BRAF mutant cell line (A2058) from multiple cancer indications were tested. The EGFR mutant cell lines were treated with single agent T-A-32, osimertinib (an EGFR inhibitor), or trametinib (a mitogen-activated protein kinase kinase [MEK] inhibitor); a dual combination of osimertinib and trametinib; or a triple combination of T-A-32, osimertinib, and trametinib in a 96-well plate format. The KRAS and BRAF mutant cell lines were treated with single agent T-A-32 or trametinib, or a dual combination of T-A-32 and trametinib in a 96-well plate format. Apoptosis induction was assessed by monitoring the activation of caspase-3/7 using a probe that generates bright green fluorescence upon DEVD peptide cleavage by activated caspase-3/7 as an early indicator of apoptosis every 2 hours for approximately 96 hours.

All cell lines were cultured in the recommended medium supplemented with 10% fetal bovine serum (FBS) at 37° C. in a humidified atmosphere containing 5% CO2 until they were at least 80% confluent as follows:
HCC4006, HCC827, and NCI-H1975 cells in Roswell Park Memorial Institute 1640+10% FBS
A2058 cells in Dulbecco's Modified Eagle Medium+10% FBS
HCT-116, Capan-2, and Calu-1 cells in McCoy's 5A+10% FBS
LoVo and A549 cells in F 12K+10% FBS

Cells were trypsinized, counted by an automated cell counter, and added to all wells of a 96-well plate at 3000 cells/well in 100 μL of the appropriate medium (1 plate for each cell line). The next day, compounds were diluted in DMSO to 1000× concentration. CELLEVENT Caspase-3/7 Green READYPROBE Reagent was prepared as per manufacturer's instructions by adding 1 drop of READYPROBE reagent into 1 mL of the appropriate medium. Compounds were diluted in the READYPROBE/medium mixture to 3× concentration and 50 μL were added to the cells. The plates were subsequently scanned every 2 hours using an Incucyte S3 Live-Cell Analysis System (Sartorius) for a total of 72 to 136 hours depending on the cell line. Plates were maintained at 37° C. in a humidified atmosphere containing 5% CO2 over the course of experiment.

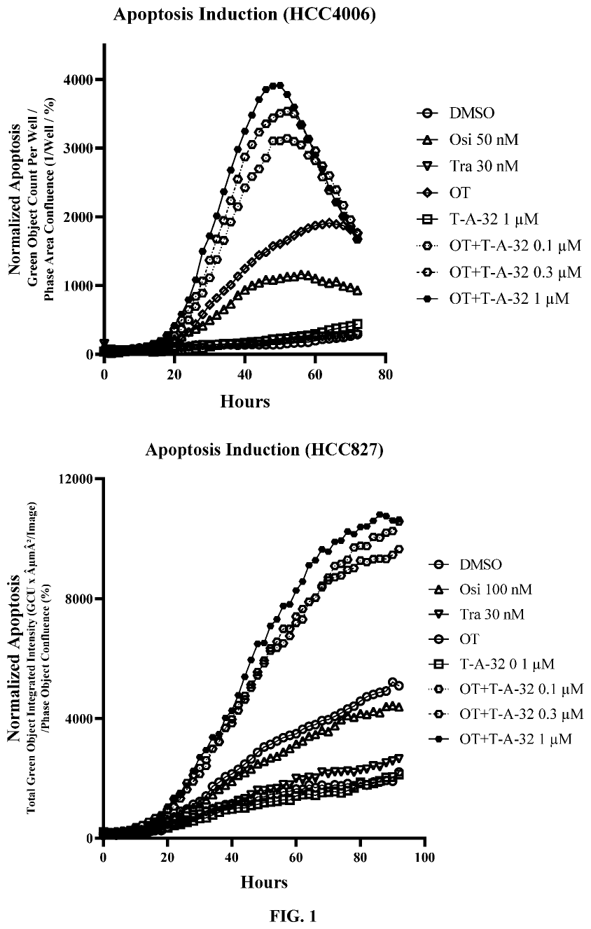

The results demonstrate that the triple combination of T-A-32, osimertinib, and trametinib enhanced apoptosis in EGFR mutant non-small cell lung cancer (NSCLC) cell lines (HCC4006 (FIG. 1), HCC827 (FIG. 1), and NCI-H1975) compared with the 3 single agent treatments and the dual combination of osimertinib and trametinib. T-A-32 in combination with trametinib enhanced apoptosis of KRAS (A549, HCT-116, Capan 2, Calu 1, and LoVo) and BRAF (A2058) mutant cell lines from multiple cancer indications compared with either single agent treatment alone.

### Example 2. The Effects of a TEAD Inhibitor T-A-32, an EGFR Inhibitor Osimertinib, an MEK Inhibitor Trametinib, and Combinations Thereof on H1975 Tumor Growth in the H1975 EGFR Mutant Lung Cancer Xenograft Mouse Model

This study determined the in vivo antitumor activity of T-A-32 administered in combination with osimertinib, an EGFR inhibitor and trametinib, a MEK inhibitor. This combination was tested in immunodeficient nude mice (Nu/Nu) bearing H1975 human non-small cell lung xenografts. H1975 was chosen as a xenograft model because H1975 harbors EGFR T790M/C797S/L858R mutations. T970M is well known point mutation for osimertinib resistance and L858R is also known EGFR activation mutation. The results demonstrated that T-A-32 in combination with both trametinib and osimertinib has significant antitumor activity compared to vehicle control in female Nu/Nu mice bearing established H1975 human non-small cell lung cancer xenografts.

Six- to 9-week-old female Nu/Nu mice were inoculated SC with 2×10⁶ H1975 human non-small cell lung tumor cells in the right flank. Tumor growth was monitored twice per week using vernier calipers and the mean tumor volume (MTV) was calculated. When the MTV reached approximately 177 mm³, animals were randomized into treatment groups (n=10/group) and dosed orally (PO) with either vehicle control (5% DMSO+95% PEG 400 (Vehicle 1)+0.5% hydroxypropyl methyl cellulose and 0.2% Tween-80 (Vehicle 2)+1% Tween 80 (Vehicle 3)) or T-A-32 at 75 mg/kg, osimertinib at 2.5 mg/kg, or trametinib at 1 mg/kg QD for 18 days.

Treatments started on Day 0, tumor size and body weight were measured twice per week, and the study was terminated when the vehicle control tumors reached a mean of approximately 1600 mm³. Percent TGI was calculated on Day 18 when the control MTV reached the maximum allowable tumor volume. The mean maximum body weight change was determined for each group.

As shown herein, treatment with T-A-32 administered PO at 75 mg/kg QD (once a day) in combination with osimertinib resulted in significant antitumor activity when compared to vehicle control (TGI=88%; p<0.001). Treatment with trametinib at 1 mg/kg in combination with osimertinib at 2.5 mg/kg resulted in significant antitumor activity compared with vehicle control (TGI=71%; p<0.001).

Treatment with T-A-32 in combination with both trametinib and osimertinib resulted in greatest significant antitumor activity (TGI=99%; p<0.001). No body weight loss was observed in this study.

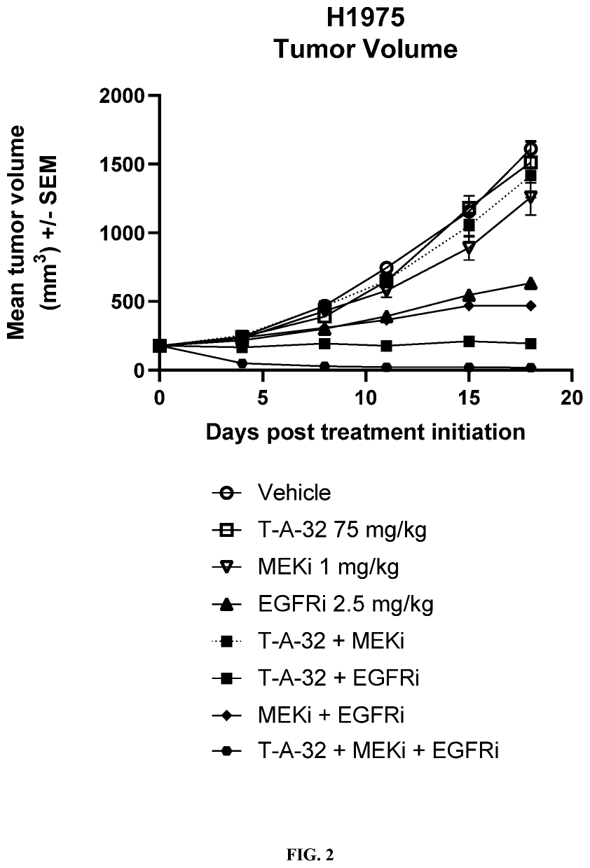

Accordingly, as shown in FIG. 2, the combination of T-A-32 and osimertinib led to synergistic effects and meaningful tumor growth inhibition, and the triple combination of T-A-32, osimertinib, and trametinib showed synergistic suppression of tumor growth leading to complete regressions in that treatment group.

### Example 3: Synthesis of Certain Exemplary TEAD Inhibitors

Certain exemplary compounds are prepared as described below.

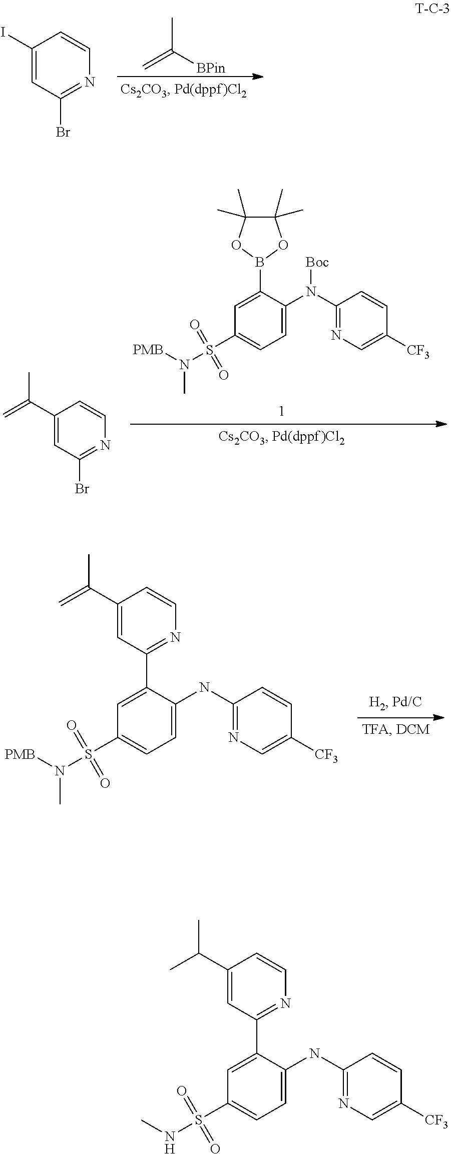

### Step 1: 2-Bromo-4-isopropenyl-pyridine

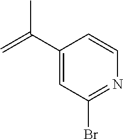

A mixture of 2-isopropenyl-4,4,5,5-tetramethyl-1,3,2-dioxaborolane (660 mg, 3.93 mmol, 1 eq), 2-bromo-4-iodo-pyridine (2 g, 7.04 mmol, 1.8 eq), Cs₂CO₃ (3.82 g, 11.73 mmol, 3 eq) and Pd(dppf)Cl₂ (143.09 mg, 195.56 μmol, 0.05 eq) in dioxane (45 mL) and H₂O (15 mL) was degassed and purged with N₂ for 3 times and the mixture was stirred under N₂ atmosphere at 100° C. for 12 h. TLC (PE/EtOAc=5/1, R_{f}=0.50) indicated 10% of starting material was remained and one major new spot with lower polarity was detected. The mixture was diluted with water (50 mL) and extracted with EtOAc (50 mL×3). The combined organic layers were dried over Na₂SO₄, filtered and concentrated under reduced pressure to yield a residue which was purified by flash silica gel chromatography (from PE/EtOAc=100/1 to 10/1, TLC: PE/EtOAc=5/1, R_{f}=0.50) to yield 2-bromo-4-isopropenyl-pyridine (650 mg, 3.15 mmol, 80.6% yield, 96.0% purity) as colorless oil. ¹H NMR (400 MHZ, CDCl₃) δ ppm 8.31 (d, J=5.1 Hz, 1H), 7.51 (d, J=1.0 Hz, 1H), 7.29 (dd, J=1.5, 5.1 Hz, 1H), 5.65-5.50 (m, 1H), 5.35-5.25 (m, 1H), 2.13 (s, 3H); ES-LCMS m z 198.1 [M+H]⁺.

### Step 2: 3-(4-Isopropenyl-2-pyridyl)-N-[(4-methoxyphenyl)methyl]-N-methyl-4-[[5-(trifluoromethyl)-2-pyridyl]amino]benzenesulfonamide

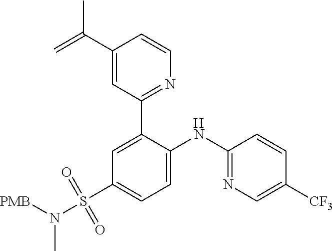

A mixture of 2-bromo-4-isopropenyl-pyridine (60 mg, 290.82 μmol, 96% purity, 1 eq), tert-butyl N-[4-[(4-methoxyphenyl)methyl-methyl-sulfamoyl]-2-(4,4,5,5-tetramethyl-1,3,2-dioxaborolan-2-yl)phenyl]-N-[5-(trifluoromethyl)-2-pyridyl]carbamate (211.16 mg, 290.46 μmol, 93.2% purity, 9.99e-1 eq), Pd(dppf)Cl₂ (19.20 mg, 26.24 μmol, 9.02e-2 eq), Cs₂CO₃ (288.00 mg, 883.93 μmol, 3.04 eq) in 1,4-dioxane (6 mL) and H₂O (2 mL) was degassed and purged with N₂ for 3 times and the mixture was stirred under N₂ atmosphere at 100° C. for 12 h. The mixture was diluted with water (10 mL) and extracted with EtOAc (10 mL×3). The combined organic layers were dried over Na₂SO₄, filtered and concentrated under reduced pressure to yield a residue which was purified by flash silica gel chromatography (from PE/EtOAc=100/1 to 4/1, TLC: PE/EtOAc=5/1, R_{f}=0.20) to yield 3-(4-isopropenyl-2-pyridyl)-N-[(4-methoxyphenyl)methyl]-N-methyl-4-[[5-(trifluoromethyl)-2-pyridyl]amino]benzenesulfonamide (140 mg, 233.90 μmol, 80.4% yield, 95.0% purity) as a light yellow solid. ¹H NMR (400 MHZ, CDCl₃) δ ppm 12.24 (s, 1H), 8.82 (d, J=8.9 Hz, 1H), 8.67 (d, J=5.3 Hz, 1H), 8.56 (s, 1H), 8.16 (d, J=2.1 Hz, 1H), 7.84 (dd, J=2.1, 8.9 Hz, 1H), 7.81-7.79 (m, 1H), 7.75 (dd, J=2.4, 8.8 Hz, 1H), 7.40 (dd, J=1.7, 5.3 Hz, 1H), 7.26-7.23 (m, 2H), 6.91 (d, J=8.7 Hz, 1H), 6.89-6.85 (m, 2H), 5.68-5.60 (m, 1H), 5.42-5.34 (m, 1H), 4.14 (s, 2H), 3.83-3.77 (m, 3H), 2.65-2.60 (m, 3H), 2.22 (s, 3H); ES-LCMS m/z 569.2 [M+H]⁺.

### Step 3: 3-(4-Isopropyl-2-pyridyl)-N-methyl-4-[[5-(trifluoromethyl)-2-pyridyl]amino]benzenesulfonamide

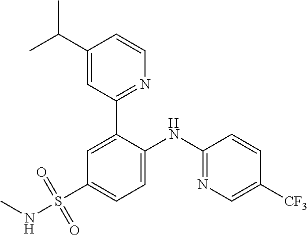

To a solution of 3-(4-isopropenyl-2-pyridyl)-N-[(4-methoxyphenyl)methyl]-N-methyl-4-[[5-(trifluoromethyl)-2-pyridyl]amino]benzenesulfonamide (140 mg, 233.90 μmol, 95% purity, 1 eq) in MeOH (50 mL) was added Pd/C (140 mg, 132.08 μmol, 10% purity, 0.5 eq). The mixture was degassed and purged with H₂ for 3 times, and then the mixture was stirred at 20° C. for 12 h under H₂ atmosphere. The mixture was filtered and concentrated under reduced pressure. The residue was added DCM (10 mL) and TFA (1 mL) and stirred at 20° C. for 12 h. The mixture was concentrated under reduced pressure and added NH₃·H₂O until pH=8 to yield a residue which was purified by preparative HPLC (column: Agela DuraShell C18 150*25 mm*5 um; mobile phase: [water (0.05% NH₃H₂O+10 mM NH₄HCO₃)-ACN]; B %: 60%-90%, 10 min), followed by lyophilization to yield 3-(4-isopropyl-2-pyridyl)-N-methyl-4-[[5-(trifluoromethyl)-2-pyridyl]amino]benzenesulfonamide (20.41 mg, 45.31 μmol, 19.4% yield, 100.0% purity) as a white solid. ¹H NMR (400 MHZ, CDCl₃) δ ppm 12.28 (br s, 1H), 8.74 (d, J=8.7 Hz, 1H), 8.61 (d, J=5.2 Hz, 1H), 8.52 (s, 1H), 8.20 (d, J=2.3 Hz, 1H), 7.86 (dd, J=2.1, 8.9 Hz, 1H), 7.73 (dd, J=2.4, 8.7 Hz, 1H), 7.67 (s, 1H), 7.27-7.24 (m, 1H), 6.90 (d, J=8.7 Hz, 1H), 4.35 (d, J=5.5 Hz, 1H), 3.03 (spt, J=6.9 Hz, 1H), 2.72 (d, J=5.5 Hz, 3H), 1.34 (d, J=6.9 Hz, 6H); ES-LCMS m/z 451.1 [M+H]⁺.

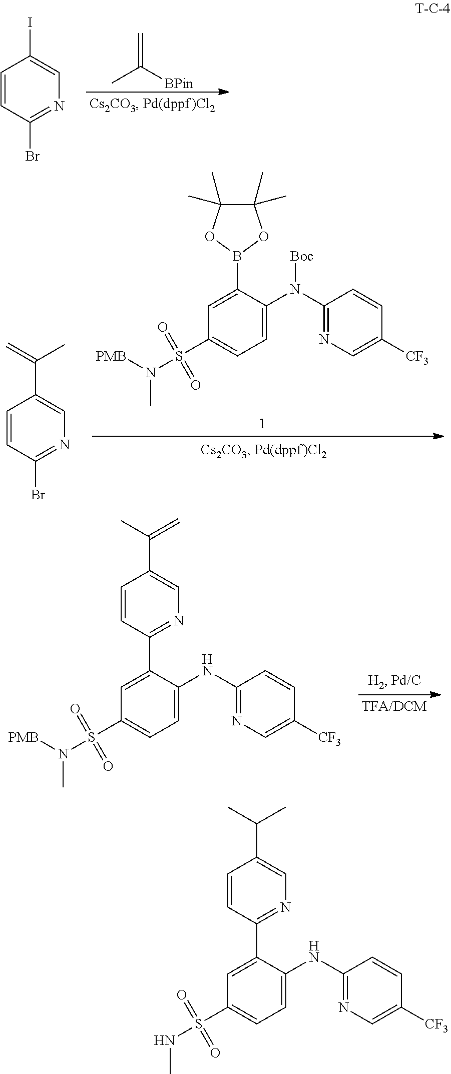

### Step 1: 2-Bromo-5-isopropenyl-pyridine

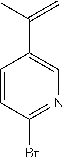

A mixture of 2-isopropenyl-4,4,5,5-tetramethyl-1,3,2-dioxaborolane (500 mg, 2.98 mmol, 1 eq), 2-bromo-5-iodo-pyridine (1.52 g, 5.35 mmol, 1.80 eq), Cs₂CO₃ (2.90 g, 8.92 mmol, 3 eq) and Pd(dppf)Cl₂ (215.86 mg, 295.01 μmol, 9.91e-2 eq) in 1,4-dioxane (60 mL) and H₂O (20 mL) was degassed and purged with N₂ for 3 times and the mixture was stirred under N₂ atmosphere at 100° C. for 12 h. TLC (PE/EtOAc=5/1, R_{f}=0.65) indicated starting material was consumed completely. The mixture was diluted with water (40 mL) and extracted with EtOAc (50 mL×3). The combined organic layers were dried over Na₂SO₄, filtered and concentrated under reduced pressure to yield a residue which was purified by flash silica gel chromatography (from PE/EtOAc=100/1 to 10/1, TLC: PE/EtOAc=5/1, R_{f}=0.65) to yield 2-bromo-5-isopropenyl-pyridine (390 mg, 1.87 mmol, 62.9% yield, 95.0% purity) as a yellow oil. ¹H NMR (400 MHZ, CDCl₃) δ ppm 8.45 (d, J=2.4 Hz, 1H), 7.61 (dd, J=2.6, 8.2 Hz, 1H), 7.44 (d, J=8.4 Hz, 1H), 5.45-5.40 (m, 1H), 5.23-5.20 (m, 1H), 2.17-2.13 (m, 3H).

### Step 2: 3-(5-Isopropenyl-2-pyridyl)-N-[(4-methoxyphenyl)methyl]-N-methyl-4-[[5-(trifluoromethyl)-2-pyridyl]amino]benzenesulfonamide

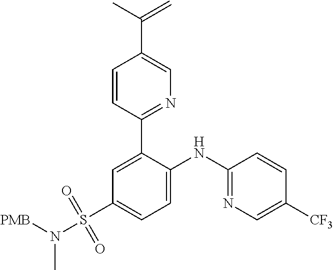

A mixture of 2-bromo-5-isopropenyl-pyridine (40 mg, 191.86 μmol, 95% purity, 1 eq), tert-butyl N-[4-[(4-methoxyphenyl)methyl-methyl-sulfamoyl]-2-(4,4,5,5-tetramethyl-1,3,2-dioxaborolan-2-yl)phenyl]-N-[5-(trifluoromethyl)-2-pyridyl]carbamate (140 mg, 185.97 μmol, 90% purity, 1 eq), Cs₂CO₃ (190.00 mg, 583.14 μmol, 3.04 eq) and Pd(dppf)Cl₂ (14 mg, 19.13 μmol, 0.1 eq) in dioxane (3 mL) and H₂O (1 mL) was degassed and purged with N₂ for 3 times and the mixture was stirred under N₂ atmosphere at 100° C. for 12 h. The mixture was diluted with water (10 mL) and extracted with EtOAc (10 mL×3). The combined organic layers were dried over Na₂SO₄, filtered and concentrated under reduced pressure to yield a residue which was purified by flash silica gel chromatography (from PE/EtOAc=100/1 to 5/1, TLC: PE/EtOAc=5/1, R_{f}=0.40) to yield 3-(5-isopropenyl-2-pyridyl)-N-[(4-methoxyphenyl)methyl]-N-methyl-4-[[5-(trifluoromethyl)-2-pyridyl]amino]benzenesulfonamide (80 mg, 137.88 μmol, 71.9% yield, 98.0% purity) as a white solid. ¹H NMR (400 MHZ, CDCl₃) δ ppm 12.53 (br s, 1H), 8.83 (dd, J=3.2, 5.5 Hz, 2H), 8.56 (s, 1H), 8.17 (d, J=2.1 Hz, 1H), 7.96 (dd, J=2.4, 8.5 Hz, 1H), 7.85-7.80 (m, 2H), 7.76 (dd, J=2.3, 8.7 Hz, 1H), 7.25 (d, J=8.5 Hz, 2H), 6.96 (d, J=8.7 Hz, 1H), 6.87 (d, J=8.5 Hz, 2H), 5.58-5.54 (m, 1H), 5.33-5.27 (m, 1H), 4.14 (s, 2H), 3.80 (s, 3H), 2.63 (s, 3H), 2.25 (s, 3H); ES-LCMS m/z 569.2 [M+H]⁺.

### Step 3: 3-(5-Isopropyl-2-pyridyl)-N-methyl-4-[[5-(trifluoromethyl)-2-pyridyl]amino]benzenesulfonamide

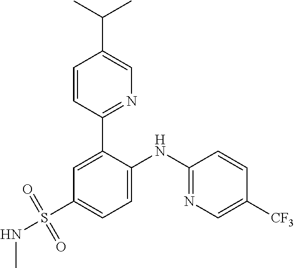 [SMILES: CNS(=O)(=O)c1ccc(Nc2ccc(C(F)(F)F)cn2)c(-c2ccc(C(C)C)cn2)c1]

To a solution of 3-(5-isopropenyl-2-pyridyl)-N-[(4-methoxyphenyl)methyl]-N-methyl-4-[[5-(trifluoromethyl)-2-pyridyl]amino]benzenesulfonamide (70 mg, 120.65 μmol, 98% purity, 1 eq) in MeOH (35 mL) was added Pd/C (70 mg, 10% purity). The mixture was degassed and purged with H₂ for 3 times and the mixture was stirred under H₂ atmosphere at 20° C. for 12 h. The mixture was filtered and concentrated under reduced pressure. The residue was dissolved in DCM (5 mL) and TFA (1 mL) and stirred at 20° C. for 12 h. The mixture was concentrated under reduced pressure and added NH₃·H₂O until pH=8 to yield a residue which was purified by preparative HPLC (column: Agela DuraShell C18 150*25 mm*5 um; mobile phase: [water (0.05% NH₃H₂O+10 mM NH₄HCO₃)-ACN]; B %: 65%-95%, 10 min), followed by lyophilization to yield 3-(5-isopropyl-2-pyridyl)-N-methyl-4-[[5-(trifluoromethyl)-2-pyridyl]amino]benzenesulfonamide (15.68 mg, 34.81 μmol, 28.9% yield, 100.0% purity) as a white solid. ¹H NMR (400 MHZ, CDCl₃) δ ppm 12.45 (br s, 1H), 8.75 (d, J=8.9 Hz, 1H), 8.59 (d, J=2.1 Hz, 1H), 8.53 (s, 1H), 8.20 (d, J=2.1 Hz, 1H), 7.85 (dd, J=2.3, 8.9 Hz, 1H), 7.83-7.80 (m, 1H), 7.80-7.76 (m, 1H), 7.74 (dd, J=2.4, 8.8 Hz, 1H), 6.93 (d, J=8.7 Hz, 1H), 4.32 (d, J=5.0 Hz, 1H), 3.05 (td, J=7.0, 13.9 Hz, 1H), 2.71 (d, J=5.5 Hz, 3H), 1.36 (d, J=6.9 Hz, 6H); ES-LCMS m/z 451.2 [M+H]⁺.

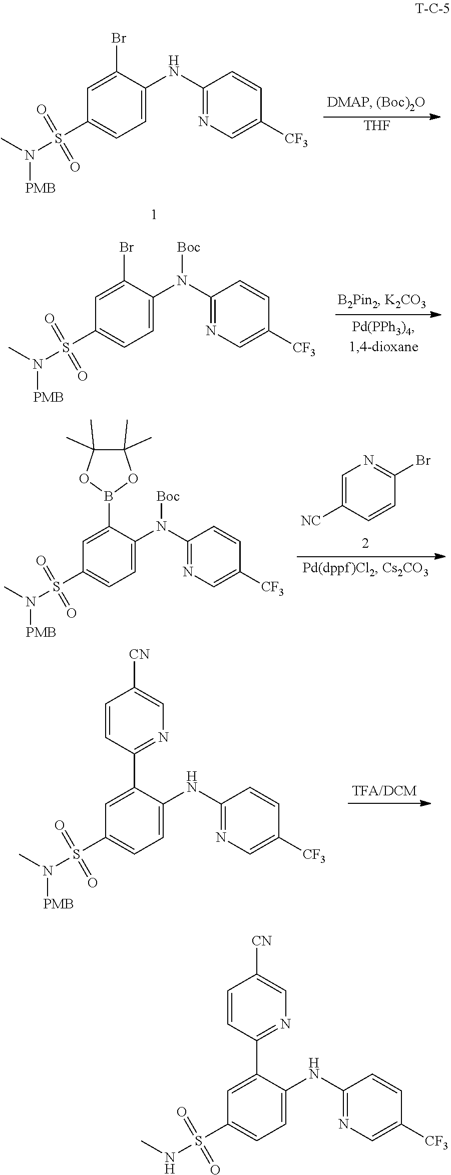

### Step 1: tert-Butyl N-[2-bromo-4-[(4-methoxyphenyl)methyl-methyl-sulfamoyl]phenyl]-N-[5-(trifluoromethyl)-2-pyridyl]carbamate

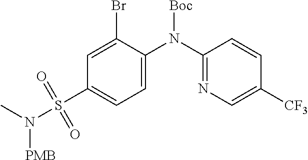

To a solution of 3-bromo-N-[(4-methoxyphenyl)methyl]-N-methyl-4-[[5-(trifluoromethyl)-2-pyridyl]amino]benzenesulfonamide (2 g, 3.69 mmol, 97.8%, 1 eq) in THF (35 mL) was added DMAP (450.57 mg, 3.69 mmol, 1 eq) and (Boc)₂O (2.41 g, 11.06 mmol, 2.54 mL, 3 eq). The mixture was stirred at 20° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.60) to yield tert-butyl N-[2-bromo-4-[(4-methoxyphenyl)methyl-methyl-sulfamoyl]phenyl]-N-[5-(trifluoromethyl)-2-pyridyl]carbamate (2.42 g, 3.68 mmol, 99.8% yield, 95.9% purity) as a white solid. ¹H NMR (400 MHZ, CDCl₃) δ ppm 8.46 (s, 1H), 8.22 (d, J=8.8 Hz, 1H), 8.11 (d, J=2.0 Hz, 1H), 7.95 (dd, J=2.1, 8.9 Hz, 1H), 7.83 (dd, J=2.0, 8.3 Hz, 1H), 7.46 (d, J=8.3 Hz, 1H), 7.24 (d, J=8.8 Hz, 2H), 6.89 (d, J=8.8 Hz, 2H), 4.17 (s, 2H), 3.82 (s, 3H), 2.67 (s, 3H), 1.45 (s, 9H); ES-LCMS m/z 630.0, 632.0 [M+H]⁺.

### Step 2: tert-Butyl N-[4-[(4-methoxyphenyl)methyl-methyl-sulfamoyl]-2-(4,4,5,5-tetramethyl-1,3,2-dioxaborolan-2-yl)phenyl]-N-[5-(trifluoromethyl)-2-pyridyl]carbamate

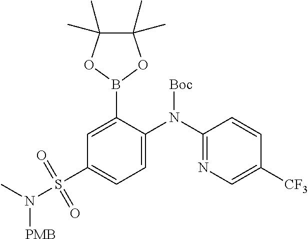

To a solution of tert-butyl N-[2-bromo-4-[(4-methoxyphenyl)methyl-methyl-sulfamoyl]phenyl]-N-[5-(trifluoromethyl)-2-pyridyl]carbamate (2.1 g, 3.19 mmol, 95.9%, 1 eq) and 4,4,5,5-tetramethyl-2-(4,4,5,5-tetramethyl-1,3,2-dioxaborolan-2-yl)-1,3,2-dioxaborolane (4.06 g, 15.97 mmol, 5 eq) in 1,4-dioxane (20 mL) was added K₂CO₃ (882.93 mg, 6.39 mmol, 2 eq) and Pd(PPh₃)+(369.12 mg, 319.43 μmol, 0.1 eq). The mixture was degassed and purged with N₂ for three times and stirred under N₂ atmosphere at 90° C. for 12 h. The reaction mixture was diluted with EtOAc (50 mL) and filtered through a pad of celite. The filtrate was concentrated under reduced pressure to give a residue. The residue was added to water (50 mL) and extracted with EtOAc (50 mL×4). The combined organic layers were dried over Na₂SO₄, filtered and the filtrate was concentrated to yield a residue which was purified by flash silica gel chromatography (from PE/EtOAc=100/1 to 3/1, TLC: PE/EtOAc=3/1, R_{f}=0.65) to yield tert-butyl N-[4-[(4-methoxyphenyl)methyl-methyl-sulfamoyl]-2-(4,4,5,5-tetramethyl-1,3,2-dioxaborolan-2-yl)phenyl]-N-[5-(trifluoromethyl)-2-pyridyl]carbamate (1.5 g, 2.06 mmol, 64.6% yield, 93.2% purity) as colorless oil. ¹H NMR (400 MHZ, CDCl₃) δ ppm 8.42 (s, 1H), 8.34-8.25 (m, 2H), 7.98-7.88 (m, 2H), 7.40 (d, J=8.3 Hz, 1H), 7.25 (d, J=8.8 Hz, 2H), 6.88 (d, J=8.6 Hz, 2H), 4.14 (s, 2H), 3.81 (s, 3H), 2.64 (s, 3H), 1.40 (s, 9H), 1.14 (s, 12H); ES-LCMS m/z 678.2 [M+H]⁺.

### Step 3: 3-(5-Cyano-2-pyridyl)-N-[(4-methoxyphenyl)methyl]-N-methyl-4-[[5-(trifluoromethyl)-2-pyridyl]amino]benzenesulfonamide

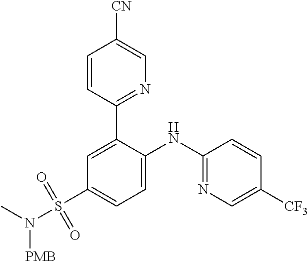

To a solution of tert-butyl N-[4-[(4-methoxyphenyl)methyl-methyl-sulfamoyl]-2-(4,4,5,5-tetramethyl-1,3,2-dioxaborolan-2-yl)phenyl]-N-[5-(trifluoromethyl)-2-pyridyl]carbamate (150 mg, 206.34 μmol, 93.2%, 1 eq) and 6-bromopyridine-3-carbonitrile (43.72 mg, 226.97 μmol, 95%, 1.1 eq) in 1,4-dioxane (6 mL) and water (2 mL) was added Cs₂CO₃ (134.46 mg, 412.67 μmol, 2 eq), Pd(dppf)Cl₂ (15.10 mg, 20.63 μmol, 0.1 eq) and 6-bromopyridine-3-carbonitrile (43.72 mg, 226.97 μmol, 95%, 1.1 eq). The mixture was bubbled with N₂ for 3 min and stirred under microwave at 100° C. for 30 min. The reaction mixture was added to saturated NaHCO₃ solution (30 mL), and extracted with DCM (30 mL×3). The combined organic layers were dried over Na₂SO₄, filtered and the filtrate was concentrated to yield a residue which was purified by flash silica gel chromatography (from PE/EtOAc=100/1 to 1/1, TLC: PE/EtOAc=1/1, R_{f}=0.45) to yield 3-(5-cyano-2-pyridyl)-N-[(4-methoxyphenyl)methyl]-N-methyl-4-[[5-(trifluoromethyl)-2-pyridyl]amino]benzenesulfonamide (40 mg, 66.19 μmol, 32.1% yield, 91.6% purity) as a yellow solid. ¹H NMR (400 MHZ, CDCl₃) δ ppm 11.90 (s, 1H), 9.02 (s, 1H), 8.83 (d, J=9.0 Hz, 1H), 8.58 (s, 1H), 8.18-8.14 (m, 2H), 7.98 (d, J=8.6 Hz, 1H), 7.89 (dd, J=1.8, 8.9 Hz, 1H), 7.81 (d, J=8.6 Hz, 1H), 7.24 (d, J=8.6 Hz, 2H), 6.96 (d, J=8.8 Hz, 1H), 6.88 (d, J=8.3 Hz, 2H), 4.15 (s, 2H), 3.81 (s, 3H), 2.65 (s, 3H); ES-LCMS m/z 554.4 [M+H]⁺.

### Step 4: 3-(5-Cyano-2-pyridyl)-N-methyl-4-[[5-(trifluoromethyl)-2-pyridyl]amino]benzenesulfonamide

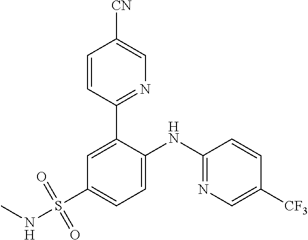 [SMILES: CNS(=O)(=O)c1ccc(Nc2ccc(C(F)(F)F)cn2)c(-c2ccc(C#N)cn2)c1]

To a stirred solution of 3-(5-cyano-2-pyridyl)-N-[(4-methoxyphenyl)methyl]-N-methyl-4-[[5-(trifluoromethyl)-2-pyridyl]amino]benzenesulfonamide (40 mg, 66.19 μmol, 91.6%, 1 eq) in DCM (3 mL) was added TFA (1.54 g, 13.51 mmol, 1 mL, 204.05 eq). The reaction mixture was stirred at 20° C. for 12 h. The reaction mixture was added to saturated NaHCO₃ solution (30 mL) and extracted with DCM (20 mL×3). The combined organic layers were dried over Na₂SO₄, filtered and the filtrate was concentrated to yield a residue which was purified by preparative HPLC (column: Agela DuraShell C18 150*25 mm*5 um; mobile phase: [water (0.05% NH₃H₂O+10 mM NH₄HCO₃)-ACN]; B %: 50%-80%, 10 min). The desired fraction was lyophilized to yield 3-(5-cyano-2-pyridyl)-N-methyl-4-[[5-(trifluoromethyl)-2-pyridyl]amino]benzenesulfonamide (15.13 mg, 34.91 μmol, 52.7% yield, 100.0% purity) as a light yellow solid. ¹H NMR (500 MHZ, DMSO-d₆) δ ppm 10.66 (s, 1H), 9.18 (d, J=1.4 Hz, 1H), 8.44 (s, 1H), 8.41 (dd, J=2.3, 8.4 Hz, 1H), 8.34 (d, J=8.9 Hz, 1H), 8.10 (d, J=2.1 Hz, 1H), 7.98 (d, J=8.4 Hz, 1H), 7.94 (dd, J=2.4, 8.9 Hz, 1H), 7.85 (dd, J=2.3, 8.7 Hz, 1H), 7.44 (d, J=5.3 Hz, 1H), 7.07 (d, J=8.7 Hz, 1H), 2.45 (d, J=4.7 Hz, 3H); ES-LCMS m/z 434.2 [M+H]⁺.

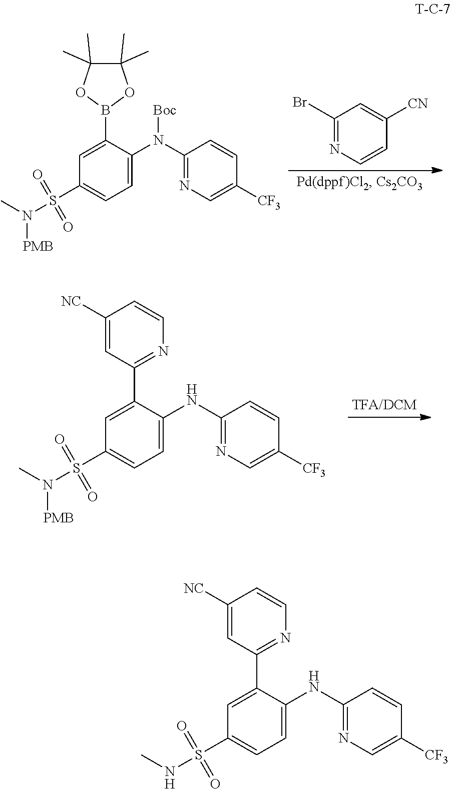

### Step 1: 3-(4-Cyano-2-pyridyl)-N-[(4-methoxyphenyl)methyl]-N-methyl-4-[[5-(trifluoromethyl)-2-pyridyl]amino]benzenesulfonamide

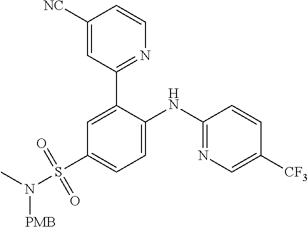

To a solution of tert-butyl N-[4-[(4-methoxyphenyl)methyl-methyl-sulfamoyl]-2-(4,4,5,5-tetramethyl-1,3,2-dioxaborolan-2-yl)phenyl]-N-[5-(trifluoromethyl)-2-pyridyl]carbamate (300 mg, 412.67 μmol, 93.2%, 1 eq) in 1,4-dioxane (6 mL) and water (2 mL) was added Cs₂CO₃ (268.91 mg, 825.34 μmol, 2 eq), Pd(dppf)Cl₂ (30.20 mg, 41.27 μmol, 0.1 eq) and 2-bromopyridine-4-carbonitrile (90.63 mg, 495.20 μmol, 1.2 eq). The mixture was bubbled with N₂ for 3 min and stirred under microwave at 100° C. for 30 min. The reaction mixture was added to saturated NaHCO₃ solution (30 mL) and extracted with EtOAc (20 mL×3). The combined organic layers were dried over Na₂SO₄, filtered and the filtrate was concentrated to yield a residue which was purified by flash silica gel chromatography (from PE/EtOAc=100/1 to 2/1, TLC: PE/EtOAc=3/1, R_{f}=0.35) to yield 3-(4-cyano-2-pyridyl)-N-[(4-methoxyphenyl)methyl]-N-methyl-4-[[5-(trifluoromethyl)-2-pyridyl]amino]benzenesulfonamide (200 mg, 325.17 μmol, 78.8% yield, 90.0% purity) as a yellow solid. ¹H NMR (500 MHZ, DMSO-d₆) δ ppm 11.02 (s, 1H), 8.98 (d, J=5.2 Hz, 1H), 8.56-8.48 (m, 2H), 8.43 (s, 1H), 8.15 (d, J=2.1 Hz, 1H), 7.97 (dd, J=2.3, 8.9 Hz, 1H), 7.93-7.89 (m, 2H), 7.25 (d, J=8.7 Hz, 2H), 7.16 (d, J=8.9 Hz, 1H), 6.92 (d, J=8.5 Hz, 2H), 4.12 (s, 2H), 3.73 (s, 3H), 2.57 (s, 3H); ES-LCMS m/z 554.4 [M+H]⁺.

### Step 2: 3-(4-Cyano-2-pyridyl)-N-methyl-4-[[5-(trifluoromethyl)-2-pyridyl]amino]benzenesulfonamide

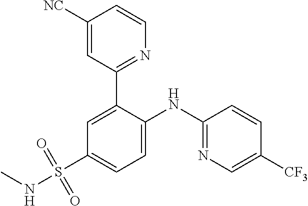 [SMILES: CNS(=O)(=O)c1ccc(Nc2ccc(C(F)(F)F)cn2)c(-c2cc(C#N)ccn2)c1]

To a stirred solution of 3-(4-cyano-2-pyridyl)-N-[(4-methoxyphenyl)methyl]-N-methyl-4-[[5-(trifluoromethyl)-2-pyridyl]amino]benzenesulfonamide (200 mg, 325.17 μmol, 90%, 1 eq) in DCM (3 mL) was added TFA (1.54 g, 13.51 mmol, 1 mL, 41.54 eq). The reaction mixture was stirred at 20° C. for 12 h. The reaction mixture was added to saturated NaHCO₃ solution (30 mL) and extracted with DCM (20 mL×3). The combined organic layers were dried over Na₂SO₄, filtered and the filtrate was concentrated to yield a residue which was purified by preparative HPLC (column: Agela DuraShell C18 150*25 mm*5 um; mobile phase: [water (0.05% NH₃H₂O+10 mM NH₄HCO₃)-ACN]; B %: 39%-69%, 10 min). The desired fraction was lyophilized to yield 3-(4-cyano-2-pyridyl)-N-methyl-4-[[5-(trifluoromethyl)-2-pyridyl]amino]benzenesulfonamide (18.1 mg, 41.76 μmol, 12.8% yield, 100.0% purity) as a white solid. ¹H NMR (500 MHZ, CDCl₃) δ ppm 11.84 (s, 1H), 8.91 (d, J=4.9 Hz, 1H), 8.76 (d, J=8.9 Hz, 1H), 8.56 (s, 1H), 8.21 (d, J=2.1 Hz, 1H), 8.12 (s, 1H), 7.92 (dd, J=2.1, 9.0 Hz, 1H), 7.78 (dd, J=2.3, 8.7 Hz, 1H), 7.59 (dd, J=1.2, 5.0 Hz, 1H), 6.93 (d, J=8.7 Hz, 1H), 4.53 (q, J=5.3 Hz, 1H), 2.74 (d, J=5.3 Hz, 3H); ES-LCMS m/z 434.1 [M+H]⁺.

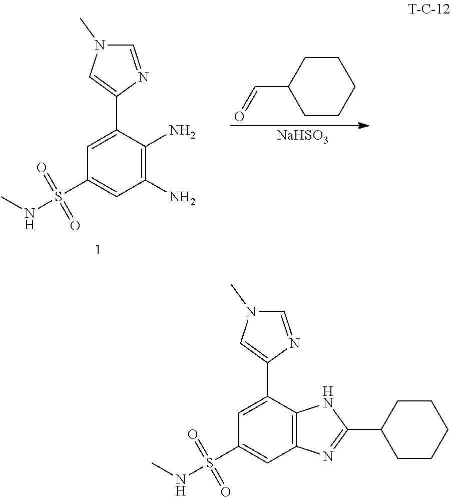

### Step 1: 2-Cyclohexyl-N-methyl-7-(1-methyl-1H-imidazol-4-yl)-1H-benzo[d]imidazole-5-sulfonamide

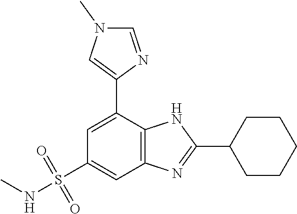 [SMILES: CNS(=O)(=O)c1cc(-c2cn(C)cn2)c2[nH]c(C3CCCCC3)nc2c1]

To a solution of 3,4-diamino-N-methyl-5-(1-methylimidazol-4-yl)benzenesulfonamide (80 mg, 255.92 μmol, 90% purity, 1 eq) in DMF (1 mL) was added sodium hydrogen sulphite (7.99 mg, 76.78 μmol, 0.3 eq), cyclohexanecarbaldehyde (30.14 mg, 268.72 μmol, 32.34 μL, 1.05 eq). The mixture was stirred under N₂ atmosphere at 140° C. for 2 h. The crude material was purified preparative HPLC ([water (10 mM NH₄HCO₃)-ACN]; B %: 26%-56%), followed by lyophilization to yield 2-cyclohexyl-N-methyl-7-(1-methylimidazol-4-yl)-1H-benzimidazole-5-sulfonamide (27.23 mg, 72.25 μmol, 28.2% yield, 99.1% purity) as a white solid. ¹H NMR (500 MHZ, DMSO-d₆) δ ppm 8.21 (s, 2H), 7.70 (s, 2H), 6.98 (s, 1H), 3.78 (s, 3H), 2.56-2.51 (m, 1H), 2.45 (s, 3H), 2.14-2.06 (m, 2H), 1.86 (d, J=3.5, 13.0 Hz, 2H), 1.78-1.65 (m, 3H), 1.50-1.41 (m, 2H), 1.40-1.32 (m, 1H); ES-LCMS m/z 374.2 [M+H]⁺.

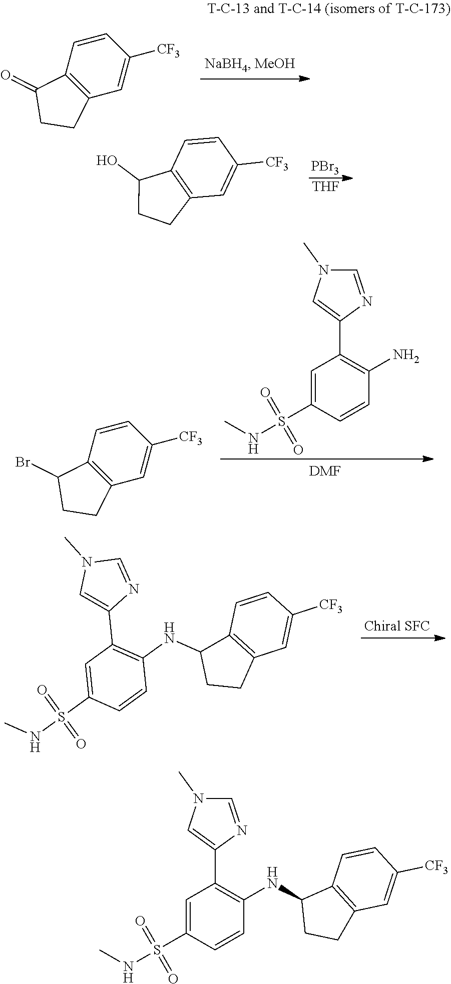

### Step 1: 5-(Trifluoromethyl)indan-1-ol

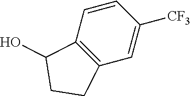 [SMILES: OC1CCc2cc(C(F)(F)F)ccc21]

To a solution of 5-(trifluoromethyl)indan-1-one (500.00 mg, 2.50 mmol, 1 eq) in THF (6 mL) was added NaBH₄ (141.75 mg, 3.75 mmol, 1.5 eq). After being stirring for 0.5 h, MeOH (2 mL) was added slowly. The mixture was stirred at 25° C. for 1 h. TLC (PE/EtOAc=10/1, R_{f}=0.48) indicated the starting material was consumed completely and one new spot formed. The reaction mixture was concentrated under reduced pressure. The residue was diluted with water (30 mL) and extracted with EtOAc (30 mL×3). The combined organic phase was washed with brine (20 mL), dried over anhydrous Na₂SO₄, filtered and concentrated in vacuum to yield 5-(trifluoromethyl)indan-1-ol (500 mg, 2.37 mmol, 95.0% yield, 96.0% purity) as yellow oil, which was used in the next step without further purification. ¹H NMR (400 MHZ, CDCl₃) δ ppm 7.52 (s, 3H), 5.29 (t, J=6.4 Hz, 1H), 3.19-3.04 (m, 1H), 2.92-2.84 (m, 1H), 2.61-2.53 (m, 1H), 2.05-1.95 (m, 1H); ES-LCMS: no desired ms was found.

### Step 2: 1-Bromo-5-(trifluoromethyl)indane

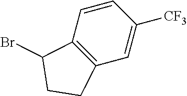 [SMILES: FC(F)(F)c1ccc2c(c1)CCC2Br]

To a solution of 5-(trifluoromethyl)indan-1-ol (410 mg, 1.95 mmol, 96%, 1 eq) in THF (10 mL) was added PBr₃ (1.58 g, 5.84 mmol, 782.92 μL, 3 eq) at 0° C. The mixture was stirred at 0° C. for 1 h. TLC (Pure PE, R_{f}=0.10) indicated the starting material was consumed completely and two new spots formed. The mixture was diluted with water (30 mL) and extracted with EtOAc (30 mL×3). The combined organic phase was washed with brine (20 mL), dried over anhydrous Na₂SO₄, filtered and concentrated in vacuum to yield 1-bromo-5-(trifluoromethyl)indane (500 mg, 1.32 mmol, 67.8% yield, 70.0% purity) as yellow oil, which was used in the next step without further purification. ¹H NMR (400 MHZ, CDCl₃) δ ppm 7.54 (d, J=7.1 Hz, 3H), 5.55 (d, J=4.9 Hz, 1H), 3.27-3.22 (m, 1H), 2.96-2.93 (m, 1H), 2.72-2.62 (m, 1H), 2.57 (d, J=6.8 Hz, 1H).

### Step 3: N-Methyl-3-(1-methylimidazol-4-yl)-4-[[(1R)-5-(trifluoromethyl)indan-1-yl]amino]benzenesulfonamide

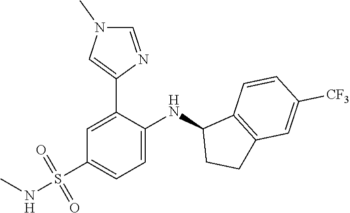 [SMILES: CNS(=O)(=O)c1ccc(N[C@@H]2CCc3cc(C(F)(F)F)ccc32)c(-c2cn(C)cn2)c1]

To a solution of 4-amino-N-methyl-3-(1-methylimidazol-4-yl)benzenesulfonamide (263.74 mg, 792.25 μmol, 80%, 1.2 eq) in DMF (3 mL) was added DIEA (255.97 mg, 1.98 mmol, 344.98 μL, 3 eq), followed by the addition of 1-bromo-5-(trifluoromethyl)indane (250.00 mg, 660.20 μmol, 70%, 1 eq). The mixture was stirred at 60° C. for 12 h. The mixture was diluted with water (50 mL) and extracted with EtOAc (50 mL×3). The combined organic phase was washed with brine (20 mL), dried over anhydrous Na₂SO₄, filtered and concentrated in vacuum to yield a residue which was purified by preparative HPLC (column: Agela DuraShell C18 150*25 mm*5 um; mobile phase: [water (0.05% NH₃·H₂O+10 mM NH₄HCO₃)-ACN]; B %: 50%-65%, 14 min), followed by lyophilization to yield product which was separated by SFC (column: DAICEL CHIRALPAK AD (250 mm*30 mm, 10 μm); mobile phase: [0.1% NH₃·H₂O MeOH]; B %: 35%-35%, min) to yield peak 1 (Rₜ=1.712 min) and peak 2 (Rₜ=2.144 min). Peak 1 was concentrated under reduced pressure to yield a residue which was dissolved in MeCN (20 mL) and H₂O (40 mL) and lyophilized to yield N-methyl-3-(1-methylimidazol-4-yl)-4-[[(1R)-5-(trifluoromethyl)indan-1-yl]amino]benzenesulfonamide (9.05 mg, 20.09 μmol, 3.0% yield, 100.0% purity, SFC: Rₜ=1.712, ee=100%, [α]^{28.2}_{D}=−44.000 (MeOH, c=0.05 g/100 mL)) as a green solid. ¹H NMR (500 MHZ, CDCl₃) δ ppm 9.12 (d, J=7.0 Hz, 1H), 7.88 (d, J=2.1 Hz, 1H), 7.62 (dd, J=2.1, 8.7 Hz, 1H), 7.54 (s, 1H), 7.52-7.46 (m, 2H), 7.39 (s, 1H), 7.28 (d, J=1.1 Hz, 1H), 6.91 (d, J=8.9 Hz, 1H), 5.16 (q, J=7.0 Hz, 1H), 4.28-4.13 (m, 1H), 3.74 (s, 3H), 3.16-3.07 (m, 1H), 3.06-2.95 (m, 1H), 2.81-2.74 (m, 1H), 2.67 (d, J=5.6 Hz, 3H), 2.11-2.02 (m, 1H); ES-LCMS m/z 451.2 [M+H]⁺.

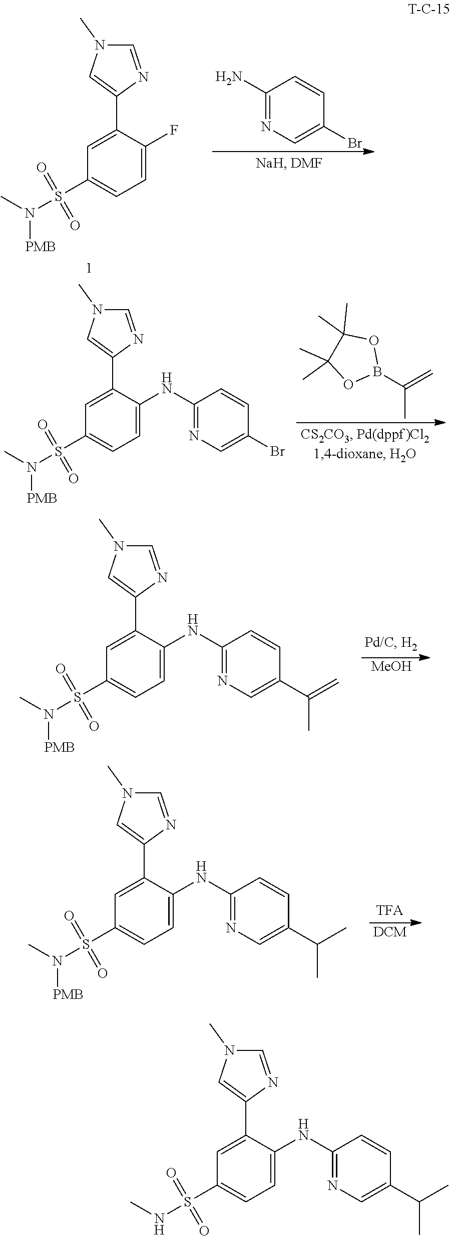

### Step 1: 4-[(5-Bromo-2-pyridyl)amino]-N-[(4-methoxyphenyl)methyl]-N-methyl-3-(1-methylimidazol-4-yl)benzenesulfonamide

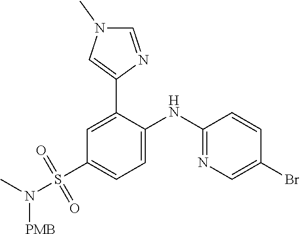

To a stirred solution of 5-bromopyridin-2-amine (319.86 mg, 1.85 mmol, 3 eq) in DMF (10 mL) was added NaH (221.83 mg, 5.55 mmol, 60%, 9 eq). The reaction mixture was stirred at 0° C. for 0.5 h. 4-Fluoro-N-[(4-methoxyphenyl)methyl]-N-methyl-3-(1-methylimidazol-4-yl)benzenesulfonamide (300 mg, 616.26 μmol, 80.0% purity, 1 eq) was added. The mixture was stirred under N₂ atmosphere at 120° C. for 11.5 h. The reaction mixture was diluted with H₂O (20 mL) and extracted with EtOAc (30 mL×3). The combined organic layers were washed with brine (20 mL), dried over Na₂SO₄, filtered and concentrated under reduced pressure to yield a residue which was purified by flash silica gel chromatography (from PE/EtOAc=100/1 to 1/1, TLC: PE/EtOAc=1/1, R_{f}=0.28) to yield 4-[(5-bromo-2-pyridyl)amino]-N-[(4-methoxyphenyl)methyl]-N-methyl-3-(1-methylimidazol-4-yl)benzenesulfonamide (180 mg, 321.87 μmol, 52.2% yield, 97.0% purity) as yellow oil. ¹H NMR (500 MHZ, CDCl₃) δ ppm 11.90 (s, 1H), 8.80 (d, J=8.9 Hz, 1H), 8.34 (d, J=2.4 Hz, 1H), 7.92 (d, J=2.3 Hz, 1H), 7.67-7.62 (m, 2H), 7.57 (s, 1H), 7.35 (d, J=1.4 Hz, 1H), 7.26-7.21 (m, 2H), 6.90-6.81 (m, 3H), 4.08 (s, 2H), 3.81 (d, J=1.1 Hz, 6H), 2.58 (s, 3H); ES-LCMS m/z 542.0, 544.0 [M+H]⁺.

### Step 2: 4-[(5-Isopropenyl-2-pyridyl)amino]-N-[(4-methoxyphenyl)methyl]-N-methyl-3-(1-methylimidazol-4-yl)benzenesulfonamide

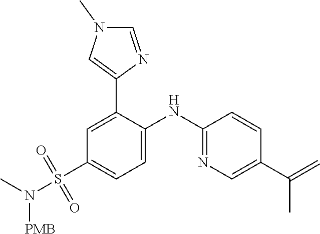

To a stirred solution of 4-[(5-bromo-2-pyridyl)amino]-N-[(4-methoxyphenyl)methyl]-N-methyl-3-(1-methylimidazol-4-yl)benzenesulfonamide (100 mg, 178.82 μmol, 97.0% purity, 1 eq) and 2-isopropenyl-4,4,5,5-tetramethyl-1,3,2-dioxaborolane (45.07 mg, 268.23 μmol, 1.5 eq) in 1,4-dioxane (6 mL) and H₂O (2 mL) was added Cs₂CO₃ (116.53 mg, 357.64 μmol, 2 eq) and Pd(dppf)Cl₂ (13.08 mg, 17.88 μmol, 0.1 eq). The reaction mixture was stirred under N₂ atmosphere at 100° C. for 3 h. The reaction mixture was diluted with H₂O (15 mL) and extracted with EtOAc (30 mL×3). The combined organic layers were washed with brine (30 mL), dried over Na₂SO₄, filtered and concentrated under reduced pressure to yield a residue which was purified by flash silica gel chromatography (from PE/EtOAc=100/1 to 1/1, TLC: PE/EtOAc=1/1, R_{f}=0.41) to yield 4-[(5-isopropenyl-2-pyridyl)amino]-N-[(4-methoxyphenyl)methyl]-N-methyl-3-(1-methylimidazol-4-yl)benzenesulfonamide (80 mg, 155.67 μmol, 87.1% yield, 98.0% purity) as yellow oil. ¹H NMR (500 MHZ, CDCl₃) δ ppm 8.39 (s, 1H), 7.96 (s, 1H), 7.75 (s, 1H), 7.68 (d, J=6.4 Hz, 1H), 7.60 (s, 1H), 7.38 (s, 1H), 7.24 (d, J=8.7 Hz, 3H), 6.96 (s, 1H), 6.87 (d, J=8.7 Hz, 2H), 5.36 (s, 1H), 5.08 (s, 1H), 4.09 (s, 2H), 3.81 (s, 6H), 2.58 (s, 3H), 2.16 (s, 3H); ES-LCMS m/z 504.2 [M+H]⁺.

### Step 3: 4-[(5-Isopropyl-2-pyridyl)amino]-N-[(4-methoxyphenyl)methyl]-N-methyl-3-(1-methylimidazol-4-yl)benzenesulfonamide

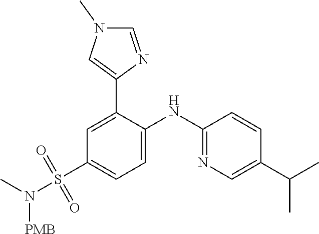

To a stirred solution of 4-[(5-isopropenyl-2-pyridyl)amino]-N-[(4-methoxyphenyl)methyl]-N-methyl-3-(1-methylimidazol-4-yl)benzenesulfonamide (80 mg, 155.67 μmol, 98.0% purity, 1 eq) in EtOAc (10 mL) was added Pd/C (100 mg, 10%). The reaction mixture was stirred under H₂ atmosphere (15 Psi) at 25° C. for 1 h. The reaction mixture was filtered and concentrated under reduced pressure to yield 4-[(5-isopropyl-2-pyridyl)amino]-N-[(4-methoxyphenyl)methyl]-N-methyl-3-(1-methylimidazol-4-yl)benzenesulfonamide (63 mg, 124.60 μmol, 80.1% yield, 100.0% purity) as a yellow solid. ¹H NMR (400 MHZ, CDCl₃) δ ppm 11.55 (s, 1H), 8.73 (d, J=8.8 Hz, 1H), 8.19 (d, J=2.2 Hz, 1H), 7.91 (d, J=2.2 Hz, 1H), 7.64 (dd, J=2.2, 8.8 Hz, 1H), 7.56 (s, 1H), 7.47 (dd, J=2.3, 8.4 Hz, 1H), 7.33 (s, 1H), 7.24 (d, J=8.6 Hz, 2H), 6.92 (d, J=8.3 Hz, 1H), 6.87 (d, J=8.6 Hz, 2H), 4.07 (s, 2H), 3.80 (d, J=3.7 Hz, 6H), 2.95-2.85 (m, 1H), 2.57 (s, 3H), 1.27 (d, J=7.1 Hz, 6H); ES-LCMS m/z 506.2 [M+H]⁺.

### Step 4: 4-[(5-Isopropyl-2-pyridyl)amino]-N-methyl-3-(1-methylimidazol-4-yl)benzenesulfonamide

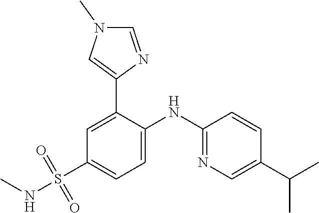 [SMILES: CNS(=O)(=O)c1ccc(Nc2ccc(C(C)C)cn2)c(-c2cn(C)cn2)c1]

To a stirred solution of 4-[(5-isopropyl-2-pyridyl)amino]-N-[(4-methoxyphenyl)methyl]-N-methyl-3-(1-methylimidazol-4-yl)benzenesulfonamide (63 mg, 124.60 μmol, 100.0% purity, 1 eq) in DCM (6 mL) was added TFA (3.08 g, 27.01 mmol, 2 mL, 216.80 eq). The reaction mixture was stirred at 25° C. for 1 h. The reaction mixture was concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 48%-78%, 10 min) to yield 4-[(5-isopropyl-2-pyridyl)amino]-N-methyl-3-(1-methylimidazol-4-yl)benzenesulfonamide (21.17 mg, 54.92 μmol, 44.1% yield, 100.0% purity) as a white solid. ¹H NMR (500 MHZ, DMSO-d₆) δ ppm 11.84 (s, 1H), 8.69 (d, J=8.9 Hz, 1H), 8.15 (d, J=2.3 Hz, 1H), 7.97-7.90 (m, 2H), 7.80 (d, J=1.1 Hz, 1H), 7.60 (dd, J=2.4, 8.5 Hz, 1H), 7.53 (dd, J=2.3, 8.9 Hz, 1H), 7.20-7.07 (m, 1H), 6.91 (d, J=8.4 Hz, 1H), 3.77 (s, 3H), 2.90-2.85 (m, 1H), 2.41 (d, J=5.0 Hz, 3H), 1.21 (d, J=6.9 Hz, 6H); ES-LCMS m/z 386.2 [M+H]⁺.

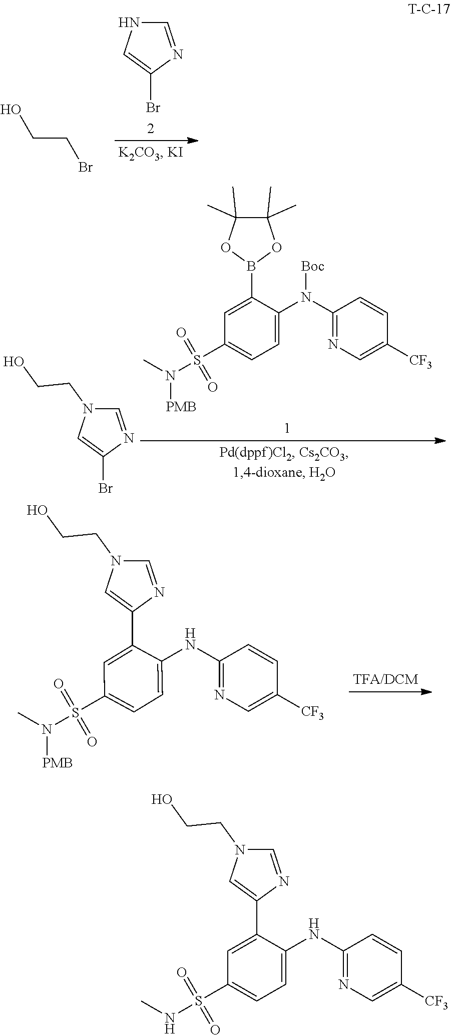

### Step 1: 2-(4-Bromoimidazol-1-yl)ethanol

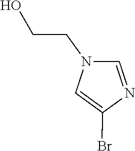

To a solution of 2-bromoethanol (2.55 g, 20.41 mmol, 1.45 mL, 3 eq) in DMF (40 mL) was added KI (1.13 g, 6.80 mmol, 1 eq), Cs₂CO₃ (8.87 g, 27.22 mmol, 4 eq) and 4-bromo-1H-imidazole (1 g, 6.80 mmol, 1 eq). The mixture was stirred under N₂ atmosphere at 100° C. for 12 h. TLC (PE/EtOAc=0/1, R_{f}=0.11) indicated starting material was consumed completely and one new spot formed. The mixture was diluted with water (80 mL) and extracted with EtOAc (50 mL×3). The combined organic layers were washed with brine (50 mL), dried over Na₂SO₄, filtered and concentrated to yield a residue which was purified by flash silica gel chromatography (From PE/EtOAc=1/0 to 0/1, R_{f}=0.11) to yield 2-(4-bromoimidazol-1-yl)ethanol (1.1 g, 3.46 mmol, 50.8% yield, 60.0% purity) as yellow oil. ¹H NMR (400 MHZ, CDCl₃) δ ppm 7.34 (d, J=1.2 Hz, 1H), 6.93 (d, J=1.6 Hz, 1H), 4.02-3.99 (m, 2H), 3.91-3.85 (m, 3H).

### Step 2: 3-[1-(2-Hydroxyethyl)imidazol-4-yl]-N-[(4-methoxyphenyl)methyl]-N-methyl-4-[[5-(trifluoromethyl)-2-pyridyl]amino]benzenesulfonamide

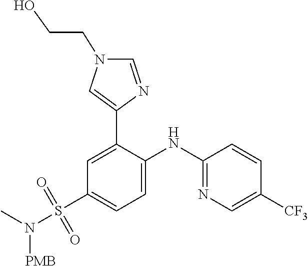

To a solution of 2-(4-bromoimidazol-1-yl)ethanol (250 mg, 785.24 μmol, 60% purity, 1 eq) in H₂O (5 mL) and 1,4-dioxane (15 mL) were added tert-butyl N-[4-[(4-methoxyphenyl)methyl-methyl-sulfamoyl]-2-(4,4,5,5-tetramethyl-1,3,2-dioxaborolan-2-yl)phenyl]-N-[5-(trifluoromethyl)-2-pyridyl]carbamate (729.80 mg, 785.24 μmol, 72.9% purity, 1 eq), Cs₂CO₃ (767.53 mg, 2.36 mmol, 3 eq) and Pd(dppf)Cl₂ (57.46 mg, 78.52 μmol, 0.1 eq). The mixture was stirred under N₂ atmosphere at 80° C. for 12 h. TLC (PE/EtOAc=1/1, R_{f}=0.10) indicated starting material was consumed completely and many new spots formed. The mixture was concentrated, diluted with water (80 mL) and extracted with EtOAc (50 mL×3). The combined organic layers were washed with brine (50 mL), dried over Na₂SO₄, filtered and concentrated to yield a residue which was purified by flash silica gel chromatography (From PE/EtOAc=1/0 to 1/1, R_{f}=0.10) to yield 3-[1-(2-hydroxyethyl)imidazol-4-yl]-N-[(4-methoxyphenyl)methyl]-N-methyl-4-[[5-(trifluoromethyl)-2-pyridyl]amino]benzenesulfonamide (50 mg, 74.61 μmol, 9.5% yield, 83.8% purity) as yellow oil. ¹H NMR (400 MHZ, CDCl₃) δ ppm 12.23 (s, 1H), 8.95 (d, J=9.0 Hz, 1H), 8.56 (s, 1H), 7.95 (d, J=2.0 Hz, 1H), 7.75-7.65 (m, 3H), 7.47 (s, 1H), 7.24 (d, J=8.6 Hz, 2H), 6.96 (d, J=8.6 Hz, 1H), 6.87 (d, J=8.6 Hz, 2H), 4.20 (t, J=5.1 Hz, 2H), 4.02 (s, 2H), 3.80 (s, 3H), 3.71 (s, 3H), 2.60 (s, 3H); ES-LCMS m/z 562.1 [M+H]⁺.

### Step 3: 3-[1-(2-Hydroxyethyl)imidazol-4-yl]-N-methyl-4-[[5-(trifluoromethyl)-2-pyridyl]amino]benzenesulfonamide

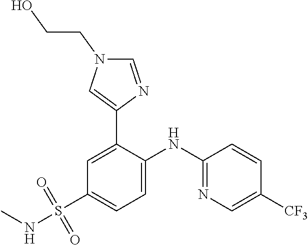 [SMILES: CNS(=O)(=O)c1ccc(Nc2ccc(C(F)(F)F)cn2)c(-c2cn(CCO)cn2)c1]

To a solution of 3-[1-(2-hydroxyethyl)imidazol-4-yl]-N-[(4-methoxyphenyl)methyl]-N-methyl-4-[[5-(trifluoromethyl)-2-pyridyl]amino]benzenesulfonamide (50 mg, 74.61 μmol, 83.8% purity, 1 eq) in DCM (4 mL) was added TFA (645.26 mg, 5.66 mmol, 419.00 μL, 75.85 eq). The mixture was stirred at 25° C. for 2 h. The mixture was concentrated, diluted with water (40 mL) and extracted with EtOAc (20 mL×3). The combined organic layers were washed with brine (30 mL), dried over Na₂SO₄, filtered and concentrated to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 34%-64%, 10 min) to yield 3-[1-(2-hydroxyethyl)imidazol-4-yl]-N-methyl-4-[[5-(trifluoromethyl)-2-pyridyl]amino]benzenesulfonamide (16.97 mg, 38.44 μmol, 51.5% yield, 100.0% purity) as a white solid. ¹H NMR (400 MHZ, CDCl₃) δ ppm 12.09 (s, 1H), 8.89 (d, J=9.0 Hz, 1H), 8.54 (s, 1H), 8.03 (d, J=2.0 Hz, 1H), 7.77-7.63 (m, 3H), 7.45 (s, 1H), 6.94 (d, J=8.6 Hz, 1H), 4.52 (s, 1H), 4.15 (d, J=4.7 Hz, 2H), 3.95 (s, 2H), 2.68 (d, J=5.5 Hz, 3H); ES-LCMS m/z 442.2 [M+H]⁺.

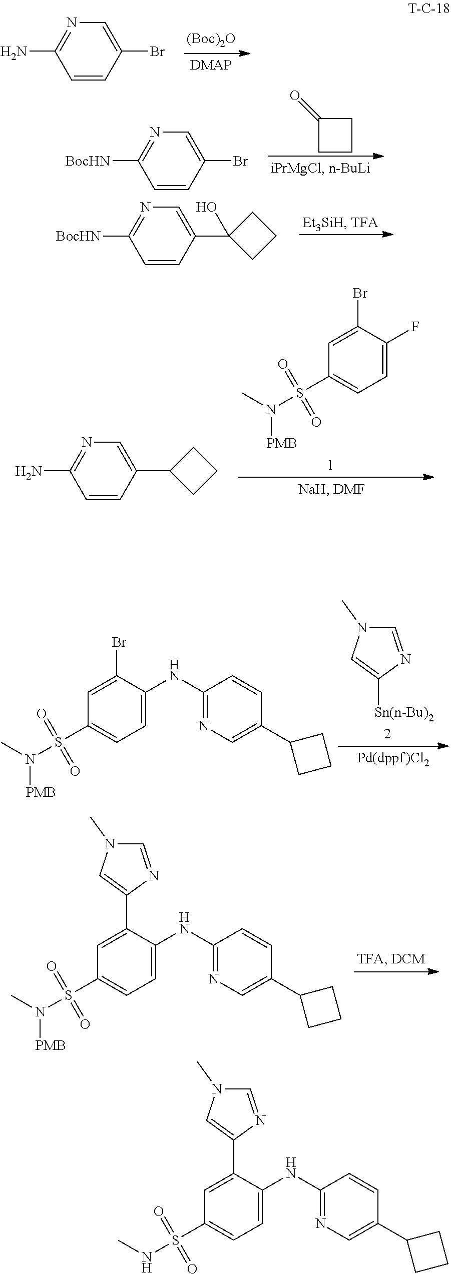

### Step 1: tert-Butyl N-(5-bromo-2-pyridyl)carbamate

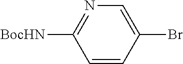 [SMILES: CC(C)(C)OC(=O)Nc1ccc(Br)cn1]

To a stirred solution of 5-bromopyridin-2-amine (4 g, 23.12 mmol, 1 eq) in DCM (60 mL) was added DMAP (4.24 g, 34.68 mmol, 1.5 eq) and (Boc)₂O (6.06 g, 27.74 mmol, 6.37 mL, 1.2 eq). The reaction mixture was stirred under N₂ atmosphere at 25° C. for 4 h. TLC (PE/EtOAc=3/1, R_{f}=0.57) showed start material was consumed completely and many new spots was detected. The reaction mixture was concentrated under reduced pressure to yield a residue which was purified by flash silica gel chromatography (from PE/EtOAc=100/1 to 100/5, TLC: PE/EtOAc=3/1, R_{f}=0.57) to yield tert-butyl N-(5-bromo-2-pyridyl)carbamate (1.9 g, 6.96 mmol, 30.1% yield, 100.0% purity) as a white solid. ¹H NMR (400 MHZ, CDCl₃) δ ppm 8.34 (s, 1H), 8.27-8.09 (m, 1H), 7.92 (d, J=9.0 Hz, 1H), 7.76 (dd, J=2.3, 8.9 Hz, 1H), 1.55 (s, 9H); ES-LCMS m/z 273.2, 275.2 [M+H]⁺.

### Step 2: tert-Butyl N-[5-(1-hydroxycyclobutyl)-2-pyridyl]carbamate

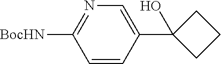 [SMILES: CC(C)(C)OC(=O)Nc1ccc(C2(O)CCC2)cn1]

To a solution of tert-butyl N-(5-bromo-2-pyridyl)carbamate (200 mg, 732.26 μmol, 100.0% purity, 1 eq) in THF (10 mL) was added i-PrMgCl (2 M, 366.13 μL, 1 eq) dropwise under N₂ atmosphere at −10° C. and stirred for 5 min. n-BuLi (2.5 M, 732.26 μL, 2.5 eq) was added dropwise under N₂ atmosphere at −30° C. and stirred for 5 min. A solution of cyclobutanone (76.99 mg, 1.10 mmol, 82.07 μL, 1.5 eq) in THF (1 mL) was added under N₂ atmosphere at −30° C. The mixture was stirred under N₂ atmosphere at −30° C. for 10 min. The reaction mixture was diluted with H₂O (20 mL) and extracted with EtOAc (30 mL×3). The combined organic layers were washed with brine (30 mL), dried over Na₂SO₄, filtered and concentrated under reduced pressure to yield a residue which was purified by flash silica gel chromatography (from PE/EtOAc=100/1 to 1/1, TLC: PE/EtOAc=1/1, R_{f}=0.28) to yield tert-butyl N-[5-(1-hydroxycyclobutyl)-2-pyridyl]carbamate (130 mg, 477.07 μmol, 65.2% yield, 97.0% purity) as a white solid. ¹H NMR (500 MHZ, DMSO-d₆) δ ppm 9.68 (s, 1H), 8.33 (d, J=2.3 Hz, 1H), 7.83-7.77 (m, 1H), 7.76-7.70 (m, 1H), 5.64-5.56 (m, 1H), 2.40-2.34 (m, 2H), 2.30-2.21 (m, 2H), 1.91-1.86 (m, 1H), 1.64-1.58 (m, 1H), 1.46 (s, 9H); ES-LCMS m/z 265.3 [M+H]⁺.

### Step 3: 5-Cyclobutylpyridin-2-amine

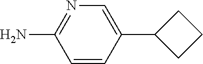 [SMILES: Nc1ccc(C2CCC2)cn1]

To a stirred solution of tert-butyl N-[5-(1-hydroxycyclobutyl)-2-pyridyl]carbamate (120 mg, 440.38 μmol, 97.0% purity, 1 eq) in triethylsilane (1.09 g, 9.39 mmol, 1.5 mL, 21.33 eq) was added TFA (2.31 g, 20.26 mmol, 1.5 mL, 46.00 eq). The reaction mixture was stirred under N₂ atmosphere at 70° C. for 1 h. The reaction mixture was concentrated under reduced pressure to yield a residue which was diluted with H₂O (10 mL), adjust pH to 9 by sat aq. NaOH and extracted with EtOAc (30 mL×3). The combined organic layers were washed with brine (20 mL), dried over Na₂SO₄, filtered and concentrated under reduced pressure to yield a residue which was purified by flash silica gel chromatography (from PE/EtOAc=100/1 to 1/1, TLC: PE/EtOAc=1/1, R_{f}=0.17) to yield 5-cyclobutylpyridin-2-amine (65 mg, 434.20 μmol, 98.6% yield, 99.0% purity) as yellow oil. ¹H NMR (500 MHZ, DMSO-d₆) δ ppm 7.74 (d, J=1.7 Hz, 1H), 7.31 (dd, J=2.3, 8.4 Hz, 1H), 6.40 (d, J=8.4 Hz, 1H), 5.67 (s, 2H), 2.22-2.16 (m, 3H), 2.04-1.96 (m, 2H), 1.92-1.86 (m, 1H), 1.80-1.73 (m, 1H); ES-LCMS m/z 149.4 [M+H]⁺.

### Step 4: 3-Bromo-4-[(5-cyclobutyl-2-pyridyl)amino]-N-[(4-methoxyphenyl)methyl]-N-methyl-benzenesulfonamide

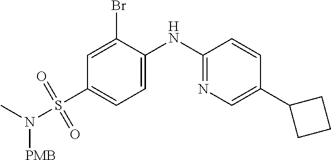

To a solution of 5-cyclobutylpyridin-2-amine (55 mg, 367.40 μmol, 99.0% purity, 1 eq) in DMF (4 mL) was added NaH (44.08 mg, 1.10 mmol, 60%, 3 eq) at 0° C. and stirred 0.5 h. 3-Bromo-4-fluoro-N-[(4-methoxyphenyl)methyl]-N-methyl-benzenesulfonamide (150.15 mg, 367.40 μmol, 95.0% purity, 1 eq) was added. The reaction mixture was stirred under N₂ atmosphere at 25° C. for 11.5 h. The reaction mixture was diluted with H₂O (20 mL) and extracted with EtOAc (40 mL×3). The combined organic layers were washed with brine (30 mL), dried over Na₂SO₄, filtered and concentrated under reduced pressure to yield a residue which was purified by flash silica gel chromatography (from PE/EtOAc=100/1 to 3/1, TLC: PE/EtOAc=3/1, R_{f}=0.56) to yield 3-bromo-4-[(5-cyclobutyl-2-pyridyl)amino]-N-[(4-methoxyphenyl)methyl]-N-methyl-benzenesulfonamide (140 mg, 265.66 μmol, 72.3% yield, 98.0% purity) as colorless oil. ¹H NMR (500 MHZ, CDCl₃) δ ppm 8.48 (d, J=8.7 Hz, 1H), 8.20 (d, J=2.1 Hz, 1H), 8.02-7.97 (m, 1H), 7.71 (dd, J=2.1, 8.8 Hz, 1H), 7.56 (dd, J=2.3, 8.4 Hz, 1H), 7.26-7.22 (m, 2H), 7.15 (s, 1H), 6.91 (d, J=8.4 Hz, 1H), 6.89-6.88 (m, 1H), 6.88-6.86 (m, 1H), 4.09 (s, 2H), 3.81 (s, 3H), 3.57-3.50 (m, 1H), 2.60-2.58 (m, 3H), 2.43-2.34 (m, 1H), 2.45-2.28 (m, 1H), 2.20-2.09 (m, 2H), 2.09-2.02 (m, 1H), 1.95-1.87 (m, 1H); ES-LCMS m/z 516.1, 518.1 [M+H]⁺.

### Step 5: 4-[(5-Cyclobutyl-2-pyridyl)amino]-N-[(4-methoxyphenyl)methyl]-N-methyl-3-(1-methylimidazol-4-yl)benzenesulfonamide

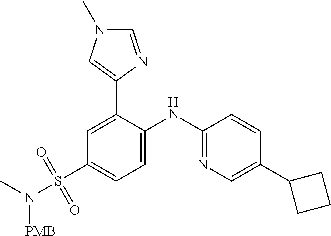

To a stirred solution of 3-bromo-4-[(5-cyclobutyl-2-pyridyl)amino]-N-[(4-methoxyphenyl)methyl]-N-methyl-benzenesulfonamide (100 mg, 189.76 μmol, 98.0% purity, 1 eq) and tributyl-(1-methylimidazol-4-yl)stannane (284.56 mg, 759.03 μmol, 99.0% purity, 4 eq) in DMF (10 mL) was added Pd(dppf)Cl₂ (13.88 mg, 18.98 μmol, 0.1 eq). The reaction mixture was stirred under N₂ atmosphere at 130° C. for 12 h. The reaction mixture was diluted with H₂O (20 mL) and extracted with EtOAc (30 mL×3). The combined organic layers were washed with brine (20 mL), dried over Na₂SO₄, filtered and concentrated under reduced pressure to yield a residue which was purified by flash silica gel chromatography (from PE/EtOAc=100/1 to 1/1, TLC: PE/EtOAc=1/1, R_{f}=0.21) to yield 4-[(5-cyclobutyl-2-pyridyl)amino]-N-[(4-methoxyphenyl)methyl]-N-methyl-3-(1-methylimidazol-4-yl)benzenesulfonamide (55 mg, 106.25 μmol, 56.0% yield, 100.0% purity) as a white solid. ¹H NMR (500 MHZ, CDCl₃) δ ppm 11.56 (s, 1H), 8.74 (d, J=8.9 Hz, 1H), 8.17 (d, J=2.1 Hz, 1H), 7.90 (d, J=2.3 Hz, 1H), 7.64 (dd, J=2.2, 8.8 Hz, 1H), 7.57 (s, 1H), 7.48 (dd, J=2.3, 8.4 Hz, 1H), 7.33 (d, J=1.1 Hz, 1H), 7.25 (s, 1H), 7.23 (s, 1H), 6.92 (d, J=8.5 Hz, 1H), 6.87 (d, J=8.7 Hz, 2H), 4.07 (s, 2H), 3.80 (d, J=2.9 Hz, 6H), 3.54-3.47 (m, 1H), 2.56 (s, 3H), 2.41-2.31 (m, 2H), 2.19-2.08 (m, 2H), 2.06-2.01 (m, 1H), 1.94-1.85 (m, 1H); ES-LCMS m/z 518.3 [M+H]⁺.

### Step 6: 4-[(5-Cyclobutyl-2-pyridyl)amino]-N-methyl-3-(1-methylimidazol-4-yl)benzenesulfonamide

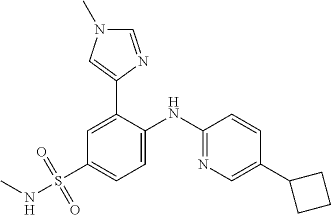 [SMILES: CNS(=O)(=O)c1ccc(Nc2ccc(C3CCC3)cn2)c(-c2cn(C)cn2)c1]

To a stirred solution of 4-[(5-cyclobutyl-2-pyridyl)amino]-N-[(4-methoxyphenyl)methyl]-N-methyl-3-(1-methylimidazol-4-yl)benzenesulfonamide (50 mg, 96.59 μmol, 100.0% purity, 1 eq) in DCM (10 mL) was added TFA (3.08 g, 27.01 mmol, 2.00 mL, 279.66 eq). The reaction mixture was stirred at 25° C. for 4 h. The reaction mixture was concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 45%-75%, 10 min) to yield 4-[(5-cyclobutyl-2-pyridyl)amino]-N-methyl-3-(1-methylimidazol-4-yl)benzenesulfonamide (34.31 mg, 86.32 μmol, 89.4% yield, 100.0% purity) as a white solid. ¹H NMR (400 MHZ, DMSO-d₆) δ ppm 11.87 (s, 1H), 8.69 (d, J=8.8 Hz, 1H), 8.12 (d, J=2.2 Hz, 1H), 7.98-7.88 (m, 2H), 7.81 (d, J=1.0 Hz, 1H), 7.63 (dd, J=2.3, 8.4 Hz, 1H), 7.53 (dd, J=2.1, 8.9 Hz, 1H), 7.20 (s, 1H), 6.92 (d, J=8.3 Hz, 1H), 3.77 (s, 3H), 3.52-3.43 (m, 1H), 2.41 (s, 3H), 2.33-2.22 (m, 2H), 2.16-2.04 (m, 2H), 2.01-1.90 (m, 1H), 1.89-1.76 (m, 1H); ES-LCMS m/z 398.3 [M+H]⁺.

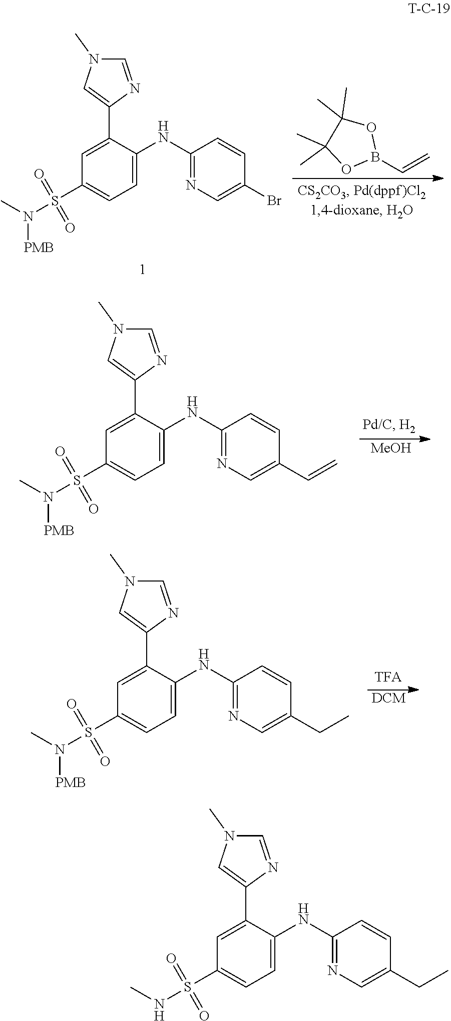

### Step 1: N-[(4-Methoxyphenyl)methyl]-N-methyl-3-(1-methylimidazol-4-yl)-4-[(5-vinyl-2-pyridyl)amino]benzenesulfonamide

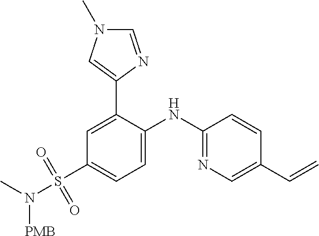

To a stirred solution of 4-[(5-bromo-2-pyridyl)amino]-N-[(4-methoxyphenyl)methyl]-N-methyl-3-(1-methylimidazol-4-yl)benzenesulfonamide (150 mg, 251.64 μmol, 91%, 1 eq) and 4,4,5,5-tetramethyl-2-vinyl-1,3,2-dioxaborolane (58.13 mg, 377.46 μmol, 64.02 μL, 1.5 eq) in 1,4-dioxane (6 mL) and H₂O (2 mL) was added Cs₂CO₃ (163.98 mg, 503.27 μmol, 2 eq) and Pd(dppf)Cl₂ (18.41 mg, 25.16 μmol, 0.1 eq). The reaction mixture was stirred under N₂ atmosphere at 100° C. for 3 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 2/1, TLC: PE/EtOAc=1/1, R_{f}=0.44) to yield N-[(4-methoxyphenyl)methyl]-N-methyl-3-(1-methylimidazol-4-yl)-4-[(5-vinyl-2-pyridyl)amino]benzenesulfonamide (110 mg, 198.03 μmol, 78.7% yield, 88.1% purity) as a white solid. ¹H NMR (400 MHZ, CDCl₃) δ ppm 11.82 (s, 1H), 8.86 (d, J=8.8 Hz, 1H), 8.29 (d, J=2.0 Hz, 1H), 7.92 (d, J=2.2 Hz, 1H), 7.68 (ddd, J=2.2, 8.7, 13.0 Hz, 2H), 7.58 (s, 1H), 7.34 (d, J=1.2 Hz, 1H), 7.24 (d, J=8.6 Hz, 2H), 6.92 (d, J=8.6 Hz, 1H), 6.87 (d, J=8.6 Hz, 2H), 6.67 (dd, J=11.0, 17.6 Hz, 1H), 5.67 (d, J=17.6 Hz, 1H), 5.22 (d, J=11.2 Hz, 1H), 4.08 (s, 2H), 3.80 (s, 6H), 2.57 (s, 3H); ES-LCMS m/z 490.2 [M+H]⁺.

### Step 2: 4-[(5-Ethyl-2-pyridyl)amino]-N-[(4-methoxyphenyl)methyl]-N-methyl-3-(1-methylimidazol-4-yl)benzenesulfonamide

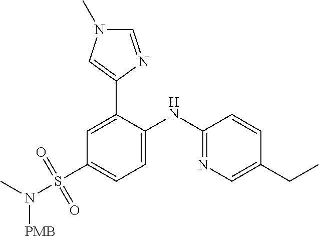

To a solution of N-[(4-methoxyphenyl)methyl]-N-methyl-3-(1-methylimidazol-4-yl)-4-[(5-vinyl-2-pyridyl)amino]benzenesulfonamide (110 mg, 198.03 μmol, 88.1%, 1 eq) in EtOAc (15 mL) was added Pd/C (100 mg, 10%, 1.00 eq) under H₂ atmosphere. The suspension was degassed and purged with H₂ for 3 times and stirred under H₂ at 25° C. for 1 h. The reaction mixture was filtered and concentrated under reduced pressure to yield compound 4-[(5-ethyl-2-pyridyl)amino]-N-[(4-methoxyphenyl)methyl]-N-methyl-3-(1-methylimidazol-4-yl)benzenesulfonamide (85 mg, 160.71 μmol, 81.1% yield, 92.9% purity) as a colorless oil, which was used in the next step without further purification. ¹H NMR (400 MHZ, CDCl₃) δ ppm 11.56 (br s, 1H), 8.74 (d, J=8.8 Hz, 1H), 8.17 (d, J=2.0 Hz, 1H), 7.90 (d, J=2.2 Hz, 1H), 7.64 (dd, J=2.2, 9.0 Hz, 1H), 7.57 (s, 1H), 7.44 (dd, J=2.3, 8.4 Hz, 1H), 7.33 (d, J=1.0 Hz, 1H), 7.24 (d, J=8.6 Hz, 2H), 6.91 (d, J=8.6 Hz, 1H), 6.87 (d, J=8.6 Hz, 2H), 4.07 (s, 2H), 3.80 (d, J=3.2 Hz, 6H), 2.64-2.58 (m, 2H), 2.56 (s, 3H), 1.27 (d, J=2.0 Hz, 3H); ES-LCMS m/z 492.2 [M+H]⁺.

### Step 3: 4-[(5-Ethyl-2-pyridyl)amino]-N-methyl-3-(1-methylimidazol-4-yl)benzenesulfonamide

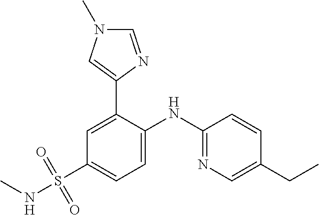 [SMILES: CCc1ccc(Nc2ccc(S(=O)(=O)NC)cc2-c2cn(C)cn2)nc1]

To a solution of 4-[(5-ethyl-2-pyridyl)amino]-N-[(4-methoxyphenyl)methyl]-N-methyl-3-(1-methylimidazol-4-yl)benzenesulfonamide (80 mg, 151.26 μmol, 92.9%, 1 eq) in DCM (6 mL) was added TFA (2.86 g, 25.11 mmol, 1.86 mL, 165.99 eq). The mixture was stirred at 25° C. for 1 h. The reaction mixture was quenched by addition of sat. NaHCO₃ (20 mL) and extracted with DCM (30 mL×3). The combined organic layers were washed with brine (10 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 39%-69%, 10 min), followed by lyophilization to yield 4-[(5-ethyl-2-pyridyl)amino]-N-methyl-3-(1-methylimidazol-4-yl)benzenesulfonamide (20 mg, 52.84 μmol, 34.9% yield, 98.1% purity) as a white solid. ¹H NMR (400 MHZ, CDCl₃) δ ppm 11.54 (s, 1H), 8.68 (d, J=9.0 Hz, 1H), 8.15 (s, 1H), 7.96 (d, J=1.7 Hz, 1H), 7.65 (dd, J=1.8, 8.9 Hz, 1H), 7.54 (s, 1H), 7.42 (dd, J=1.7, 8.3 Hz, 1H), 7.32 (s, 1H), 6.89 (d, J=8.3 Hz, 1H), 4.36 (d, J=5.4 Hz, 1H), 3.77 (s, 3H), 2.65 (d, J=5.4 Hz, 3H), 2.59 (q, J=7.6 Hz, 2H), 1.24 (t, J=7.6 Hz, 3H); ES-LCMS m/z 372.2 [M+H]⁺.

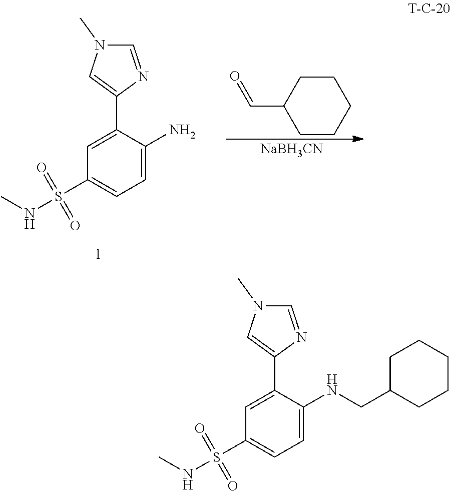

### Step 1: 4-(Cyclohexylmethylamino)-N-methyl-3-(1-methylimidazol-4-yl)benzenesulfonamide

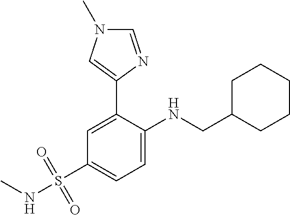 [SMILES: CNS(=O)(=O)c1ccc(NCC2CCCCC2)c(-c2cn(C)cn2)c1]

A mixture of 4-amino-N-methyl-3-(1-methylimidazol-4-yl)benzenesulfonamide (100 mg, 300.39 μmol, 80% purity, 1 eq) and cyclohexanecarbaldehyde (42 mg, 374.43 μmol, 45.06 μL, 1.25 eq) in MeOH (3 mL) and AcOH (0.05 mL) was stirred at 25° C. for 3 h. NaBH₃CN (56.00 mg, 891.12 μmol, 2.97 eq) was added. The mixture was stirred at 25° C. for 12 h. The mixture was filtered. The solid was stirred in MeOH (4 mL) for 0.5 h and filtered. The solid was washed with MeOH (2 mL), dissolved in MeCN (50 mL) and water (50 mL) and lyophilized to yield 4-(cyclohexylmethylamino)-N-methyl-3-(1-methylimidazol-4-yl)benzenesulfonamide (27.35 mg, 75.45 μmol, 25.1% yield, 100.0% purity) as a white solid. ¹H NMR (500 MHZ, CDCl₃) δ ppm 8.63 (br s, 1H), 7.81 (d, J=2.3 Hz, 1H), 7.56 (dd, J=2.2, 8.8 Hz, 1H), 7.48 (s, 1H), 7.25 (d, J=1.1 Hz, 1H), 6.66 (d, J=8.9 Hz, 1H), 4.16 (q, J=5.5 Hz, 1H), 3.76 (s, 3H), 3.09 (t, J=6.0 Hz, 2H), 2.62 (d, J=5.5 Hz, 3H), 1.87 (d, J=12.8 Hz, 2H), 1.80-1.74 (m, 2H), 1.73-1.64 (m, 2H), 1.31-1.18 (m, 3H), 1.05 (dq, J=3.2, 12.1 Hz, 2H); ES-LCMS m/z 363.3 [M+H]⁺.

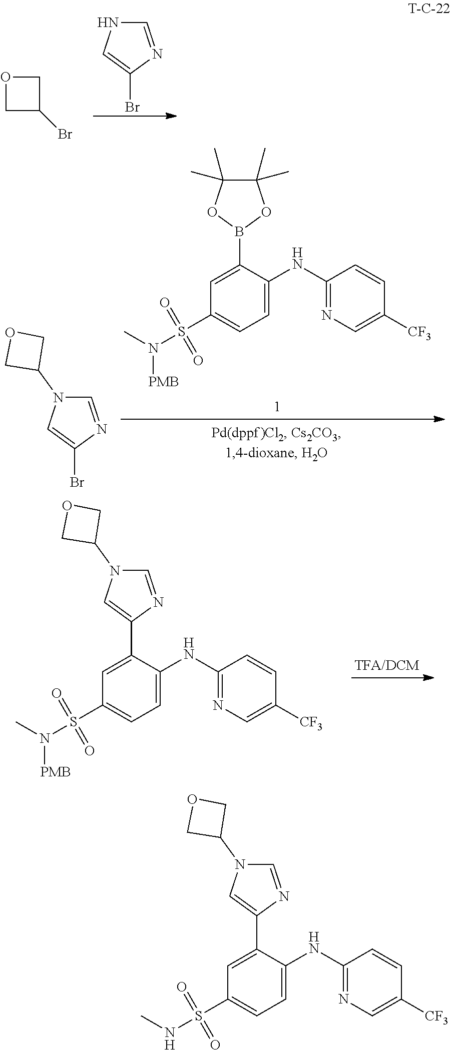

### Step 1: 4-Bromo-1-(oxetan-3-yl)imidazole

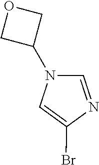

To a solution of 3-bromooxetane (700 mg, 5.11 mmol, 1 eq) in DMF (20 mL) was added Cs₂CO₃ (3.33 g, 10.22 mmol, 2 eq) and 4-bromo-1H-imidazole (1.13 g, 7.67 mmol, 1.5 eq). The mixture was stirred at 130° C. for 8 h. The reaction mixture was diluted with water (30 mL) and extracted with EtOAc (50 mL×3). The combined organic phases were washed with brine (50 mL), dried over anhydrous Na₂SO₄, filtered and concentrated under reduced pressure to yield a residue which was purified by flash silica gel chromatography (from PE/EtOAc=1/0 to 2/1, TLC: PE/EtOAc=0/1, R_{f}=0.57) to yield 4-bromo-1-(oxetan-3-yl)imidazole (740 mg, 3.23 mmol, 63.1% yield, 88.5% purity) as a yellow solid. ¹H NMR (400 MHZ, CDCl₃) δ ppm 7.51 (d, J=1.5 Hz, 1H), 7.28 (d, J=1.5 Hz, 1H), 5.31-5.22 (m, 1H), 5.11 (t, J=7.3 Hz, 2H), 4.85-4.79 (m, 2H); ES-LCMS m/z 204.9 [M+H]⁺.

### Step 2: N-[(4-Methoxyphenyl)methyl]-N-methyl-3-[1-(oxetan-3-yl)imidazol-4-yl]-4-[[5-(trifluoromethyl)-2-pyridyl]amino]benzenesulfonamide

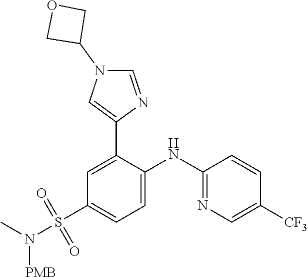

To a solution of 4-bromo-1-(oxetan-3-yl)imidazole (200 mg, 871.77 μmol, 88.5% purity, 1 eq) in 1,4-dioxane (5 mL) and H₂O (1 mL) was added Pd(dppf)Cl₂ (6.38 mg, 8.72 μmol, 0.01 eq), Cs₂CO₃ (568.08 mg, 1.74 mmol, 2 eq) and tert-butyl N-[4-[(4-methoxyphenyl)methyl-methyl-sulfamoyl]-2-(4,4,5,5-tetramethyl-1,3,2-dioxaborolan-2-yl)phenyl]-N-[5-(trifluoromethyl)-2-pyridyl]carbamate (1.42 g, 1.05 mmol, 50% purity, 1.2 eq). The mixture was stirred at 100° C. for 5 h. The reaction mixture was diluted with water (20 mL) and extracted with EtOAc (30 mL×3). The combined organic phases were washed with brine (20 mL), dried over anhydrous Na₂SO₄, filtered and concentrated under reduced pressure to yield a residue which was purified by flash silica gel chromatography (from PE/EtOAc=1/0 to 1/1, TLC: PE/EtOAc=2/1, R_{f}=0.21) to yield N-[(4-methoxyphenyl)methyl]-N-methyl-3-[1-(oxetan-3-yl)imidazol-4-yl]-4-[[5-(trifluoromethyl)-2-pyridyl]amino]benzenesulfonamide (130 mg, 113.32 μmol, 13.0% yield, 50.0% purity) as a yellow solid. ¹H NMR (400 MHZ, CDCl₃) δ ppm 12.05 (s, 1H), 8.93 (d, J=9.0 Hz, 1H), 8.53 (s, 1H), 7.98 (d, J=2.2 Hz, 1H), 7.78 (d, J=1.0 Hz, 1H), 7.71-7.67 (m, 2H), 7.16 (d, J=8.3 Hz, 2H), 6.96-6.88 (m, 2H), 6.85 (d, J=8.8 Hz, 2H), 5.40-5.31 (m, 1H), 5.17 (t, J=7.3 Hz, 2H), 4.90 (d, J=6.0, 7.2 Hz, 2H), 4.09 (s, 2H), 3.78 (s, 3H), 2.59 (s, 3H); ES-LCMS m/z 574.2 [M+H]⁺.

### Step 3: N-Methyl-3-[1-(oxetan-3-yl)imidazol-4-yl]-4-[[5-(trifluoromethyl)-2-pyridyl]amino]benzenesulfonamide

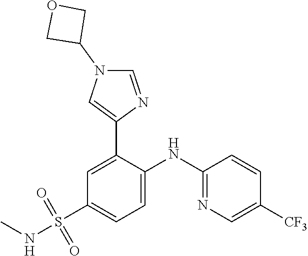 [SMILES: CNS(=O)(=O)c1ccc(Nc2ccc(C(F)(F)F)cn2)c(-c2cn(C3COC3)cn2)c1]

To a solution of N-[(4-methoxyphenyl)methyl]-N-methyl-3-[1-(oxetan-3-yl)imidazol-4-yl]-4-[[5-(trifluoromethyl)-2-pyridyl]amino]benzenesulfonamide (120 mg, 104.61 μmol, 50% purity, 1 eq) in DCM (3 mL) was added TFA (462.00 mg, 4.05 mmol, 300.00 μL, 38.74 eq). The mixture was stirred at 25° C. for 1 h. The reaction mixture was concentrated to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 44%-59%, 14 min), followed by lyophilization to yield N-methyl-3-[1-(oxetan-3-yl)imidazol-4-yl]-4-[[5-(trifluoromethyl)-2-pyridyl]amino]benzenesulfonamide (19.77 mg, 43.60 μmol, 41.7% yield, 100.0% purity) as a yellow solid. ¹H NMR (400 MHZ, CDCl₃) δ ppm 12.04 (s, 1H), 8.90 (d, J=9.0 Hz, 1H), 8.55 (s, 1H), 8.07 (d, J=2.2 Hz, 1H), 7.78 (s, 2H), 7.74 (td, J=2.8, 8.7 Hz, 2H), 6.94 (d, J=8.8 Hz, 1H), 5.43-5.34 (m, 1H), 5.20 (t, J=7.3 Hz, 2H), 4.96-4.89 (m, 2H), 4.31 (q, J=5.3 Hz, 1H), 2.70 (d, J=5.4 Hz, 3H); ES-LCMS m/z 454.2 [M+H]⁺.

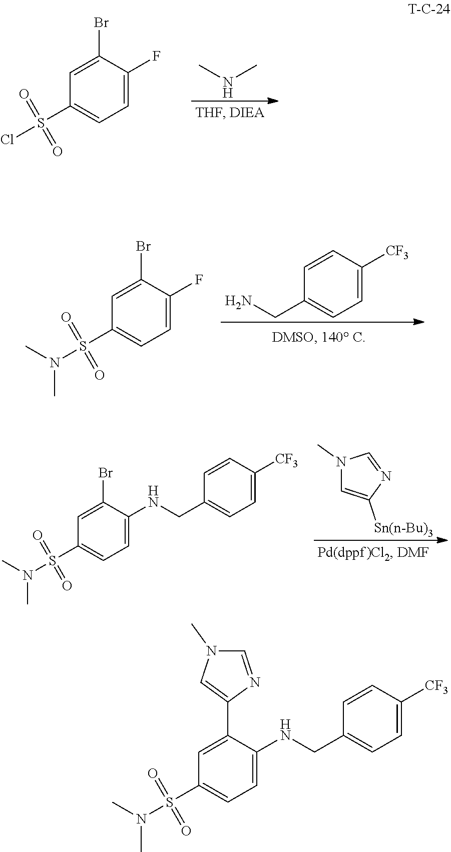

### Step 1: 3-Bromo-4-fluoro-N,N-dimethyl-benzenesulfonamide

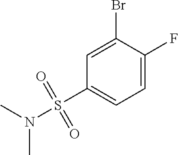

To a solution of 3-bromo-4-fluoro-benzenesulfonyl chloride (300 mg, 1.10 mmol, 1 eq) in THF (6 mL) was added DIEA (425.29 mg, 3.29 mmol, 573.16 μL, 3 eq) and N-methylmethanamine (185.44 mg, 1.65 mmol, 208.36 μL, 40%, 1.5 eq). The mixture was stirred at 20° C. for 1 h. TLC (PE/EtOAc=3/1, R_{f}=0.40) indicated most of the starting material was consumed and one new spot formed. The reaction mixture was concentrated to yield a residue which was purified by flash silica gel chromatography (from PE/EtOAc=1/0 to 3/1, TLC: PE/EtOAc=3/1, R_{f}=0.40) to yield 3-bromo-4-fluoro-N,N-dimethyl-benzenesulfonamide (300 mg, 1.03 mmol, 94.0% yield, 97.0% purity) as a white solid. ¹H NMR (400 MHZ, CDCl₃) δ ppm 8.01 (dd, J=2.2, 6.4 Hz, 1H), 7.73 (m, 1H), 7.32-7.28 (m, 1H), 2.74 (s, 6H); ES-LCMS m/z 282.1, 284.1 [M+H]⁺.

### Step 2: 3-Bromo-N,N-dimethyl-4-[[4-(trifluoromethyl)phenyl]methylamino]benzenesulfonamide

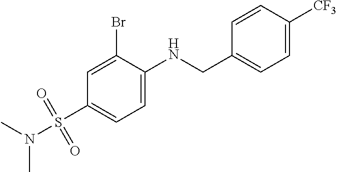 [SMILES: CN(C)S(=O)(=O)c1ccc(NCc2ccc(C(F)(F)F)cc2)c(Br)c1]

A mixture of 3-bromo-4-fluoro-N,N-dimethyl-benzenesulfonamide (300 mg, 1.03 mmol, 97%, 1 eq), [4-(trifluoromethyl)phenyl]methanamine (361.32 mg, 2.06 mmol, 293.75 μL, 2 eq) in DMSO (15 mL) was degassed and purged with N₂ for 3 times and stirred under N₂ atmosphere at 140° C. for 4 h. The reaction mixture was diluted with water (20 mL) and extracted with EtOAc (50 mL×3). The combined organic layers were dried over Na₂SO₄, filtered and concentrated to yield a residue which was purified by flash silica gel chromatography (from PE/EtOAc=100/1 to 3/1, TLC: PE/EtOAc=3/1, R_{f}=0.41) to yield 3-bromo-N,N-dimethyl-4-[[4-(trifluoromethyl)phenyl]methylamino]benzenesulfonamide (310 mg, 574.24 μmol, 55.6% yield, 81.0% purity) as a white solid. ¹H NMR (400 MHZ, CDCl₃) δ ppm 7.88 (d, J=2.0 Hz, 1H), 7.64 (d, J=8.2 Hz, 2H), 7.52 (dd, J=2.0, 8.6 Hz, 1H), 7.47 (d, J=7.8 Hz, 2H), 6.56 (d, J=8.6 Hz, 1H), 5.35 (t, J=5.3 Hz, 1H), 4.56 (d, J=5.5 Hz, 2H), 2.69 (s, 6H); ES-LCMS m/z 437.1, 439.1 [M+H]⁺.

### Step 3: N,N-Dimethyl-3-(1-methylimidazol-4-yl)-4-[[4-(trifluoromethyl)phenyl]methylamino]benzenesulfonamide

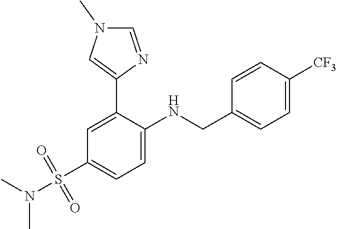 [SMILES: CN(C)S(=O)(=O)c1ccc(NCc2ccc(C(F)(F)F)cc2)c(-c2cn(C)cn2)c1]

To a solution of 3-bromo-N,N-dimethyl-4-[[4-(trifluoromethyl)phenyl]methylamino]benzenesulfonamide (310 mg, 574.24 μmol, 81%, 1 eq) in DMF (4 mL) was added Pd(dppf)Cl₂ (42.02 mg, 57.42 μmol, 0.1 eq) and tributyl-(1-methylimidazol-4-yl)stannane (538.20 mg, 1.44 mmol, 99%, 2.5 eq). The mixture was stirred under N₂ atmosphere at 130° C. for 3 h. The reaction mixture was diluted with water (20 mL) and extracted with EtOAc (50 mL×3). The combined organic layers were dried over Na₂SO₄, filtered and concentrated to yield a residue which was purified by flash silica gel chromatography (from PE/EtOAc=0/1 to 1/1, TLC: PE/EtOAc=1/1, R_{f}=0.20) to yield N,N-dimethyl-3-(1-methylimidazol-4-yl)-4-[[4-(trifluoromethyl)phenyl]methylamino]benzenesulfonamide (135.21 mg, 302.20 μmol, 52.6% yield, 98.0% purity) as a white solid. ¹H NMR (400 MHZ, CDCl₃) δ ppm 9.20 (s, 1H), 7.78 (d, J=2.0 Hz, 1H), 7.60 (d, J=8.2 Hz, 2H), 7.50 (d, J=7.8 Hz, 3H), 7.43 (dd, J=2.0, 8.6 Hz, 1H), 7.31 (d, J=1.2 Hz, 1H), 6.56 (d, J=9.0 Hz, 1H), 4.60 (d, J=5.5 Hz, 2H), 3.79 (s, 3H), 2.67 (s, 6H); ES-LCMS m/z 439.2 [M+H]⁺.

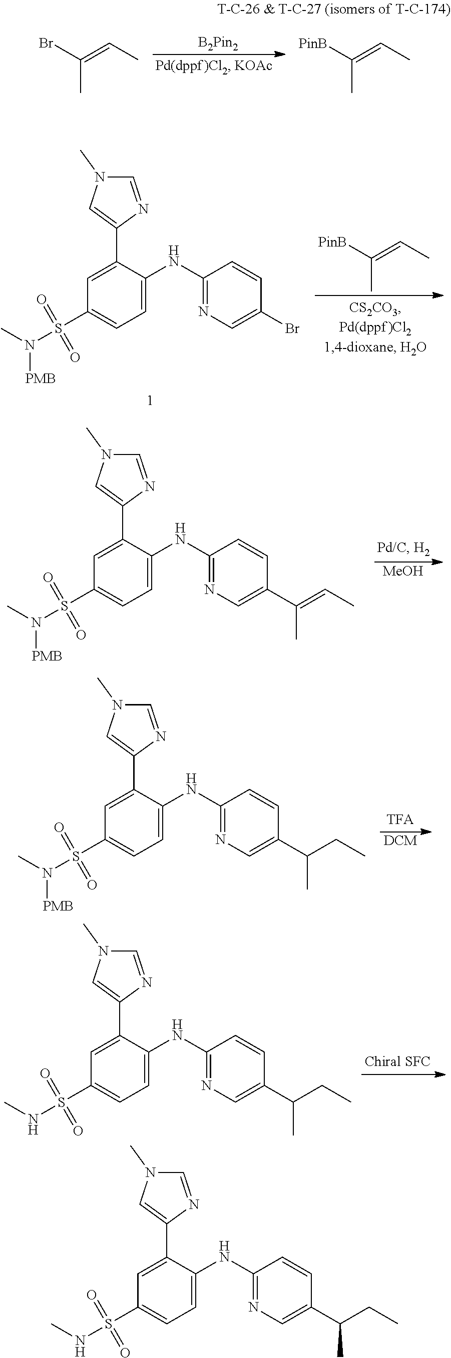

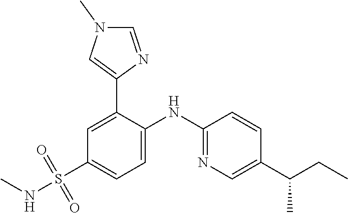 [SMILES: CC[C@H](C)c1ccc(Nc2ccc(S(=O)(=O)NC)cc2-c2cn(C)cn2)nc1]

### Step 1: 4,4,5,5-Tetramethyl-2-[(Z)-1-methylprop-1-enyl]-1,3,2-dioxaborolane

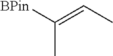

A stirred solution of (E)-2-bromobut-2-ene (0.4 g, 2.96 mmol, 1 eq) in THF (10 mL) was cooled to −78° C. and t-BuLi (1.3 M, 4.10 mL, 1.8 eq) was added dropwise. The resulting mixture was stirred at −78° C. for 30 minutes and 2-isopropoxy-4,4,5,5-tetramethyl-1,3,2-dioxaborolane (661.52 mg, 3.56 mmol, 725.35 μL, 1.2 eq) was added dropwise. The resulting mixture was stirred at −78° C. for 30 minutes and warmed to 25° C. and stirred for 1 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield 4,4,5,5-tetramethyl-2-[(Z)-1-methylprop-1-enyl]-1,3,2-dioxaborolane (400 mg, 1.76 mmol, 59.3% yield, 80.0% purity) as a yellow oil, which was used in the next step without further purification. ¹H NMR (400 MHZ, CDCl₃) δ ppm 6.15 (d, J=6.1 Hz, 1H), 1.88 (dd, J=1.5, 6.8 Hz, 3H), 1.75 (s, 3H), 1.28 (s, 12H).

### Step 2: N-[(4-Methoxyphenyl)methyl]-N-methyl-3-(1-methylimidazol-4-yl)-4-[[5-[(E)-1-methylprop-1-enyl]-2-pyridyl]amino]benzenesulfonamide

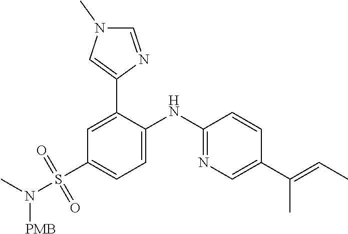

To a solution of 4-[(5-bromo-2-pyridyl)amino]-N-[(4-methoxyphenyl)methyl]-N-methyl-3-(1-methylimidazol-4-yl)benzenesulfonamide (220 mg, 369.07 μmol, 91%, 1 eq) in H₂O (2 mL) and 1,4-dioxane (8 mL) was added Pd(dppf)Cl₂ (27.01 mg, 36.91 μmol, 0.1 eq), 4,4,5,5-tetramethyl-2-[(E)-1-methylprop-1-enyl]-1,3-dioxolane (255.04 mg, 1.11 mmol, 80%, 3 eq) and Cs₂CO₃ (240.50 mg, 738.14 μmol, 2 eq). The mixture was stirred at 100° C. for 3 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 2/1, TLC: PE/EtOAc=2/1, R_{f}=0.49) to yield N-[(4-methoxyphenyl)methyl]-N-methyl-3-(1-methylimidazol-4-yl)-4-[[5-[(E)-1-methylprop-1-enyl]-2-pyridyl]amino]benzenesulfonamide (175 mg, 328.81 μmol, 89.0% yield, 97.2% purity) as a colorless oil. ¹H NMR (400 MHZ, CDCl₃) δ ppm 11.69-11.63 (m, 1H), 8.82-8.75 (m, 1H), 8.33-8.15 (m, 1H), 7.91-7.87 (m, 1H), 7.66-7.60 (m, 1H), 7.54 (s, 1H), 7.42 (dd, J=2.3, 8.4 Hz, 1H), 7.31 (d, J=1.0 Hz, 1H), 7.21 (d, J=8.6 Hz, 2H), 6.93-6.81 (m, 3H), 5.86-0.56 (m, 1H), 4.05 (s, 2H), 3.78 (s, 6H), 2.54 (s, 3H), 2.00 (s, 3H), 1.22 (s, 3H); ES-LCMS m/z 518.2 [M+H]⁺.

### Step 3: N-[(4-Methoxyphenyl)methyl]-N-methyl-3-(1-methylimidazol-4-yl)-4-[(5-sec-butyl-2-pyridyl)amino]benzenesulfonamide

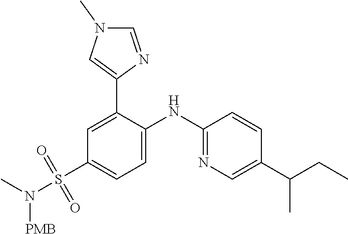

To a solution of N-[(4-methoxyphenyl)methyl]-N-methyl-3-(1-methylimidazol-4-yl)-4-[[5-[(E)-1-methylprop-1-enyl]-2-pyridyl]amino]benzenesulfonamide (170 mg, 299.71 μmol, 91.2%, 1 eq) in EtOAc (10 mL) was added Pd/C (100 mg, 10%) under H₂ atmosphere. The mixture was stirred under H₂ (15 Psi) at 25° C. for 1 h. The reaction mixture was filtered and concentrated under reduced pressure to yield N-[(4-methoxyphenyl)methyl]-N-methyl-3-(1-methylimidazol-4-yl)-4-[(5-sec-butyl-2-pyridyl)amino]benzenesulfonamide (150 mg, 259.79 μmol, 86.6% yield, 90.0% purity) as a colorless oil. ¹H NMR (400 MHZ, CDCl₃) δ ppm 11.56 (s, 1H), 8.76 (d, J=9.0 Hz, 1H), 8.14 (d, J=2.0 Hz, 1H), 7.91-7.90 (m, 1H), 7.66 (dd, J=2.4, 5.6 Hz, 1H), 7.56 (s, 1H), 7.42 (dd, J=2.3, 8.4 Hz, 1H), 7.35-7.33 (m, 2H), 7.29 (s, 1H), 6.87 (d, J=8.6 Hz, 2H), 6.66 (d, J=8.6 Hz, 1H), 4.07 (s, 2H), 3.80 (s, 3H), 3.79 (s, 3H), 2.64 (d, J=5.6 Hz, 3H), 1.60-1.53 (m, 2H), 1.26-1.25 (m, 3H), 0.85 (t, J=7.3 Hz, 3H); ES-LCMS m/z 520.3 [M+H]⁺.

### Step 4: N-Methyl-3-(1-methylimidazol-4-yl)-4-[[5-[(1R)-1-methylpropyl]-2-pyridyl]amino]benzenesulfonamide and N-methyl-3-(1-methylimidazol-4-yl)-4-[[5-[(1S)-1-methylpropyl]-2-pyridyl]amino]benzenesulfonamide

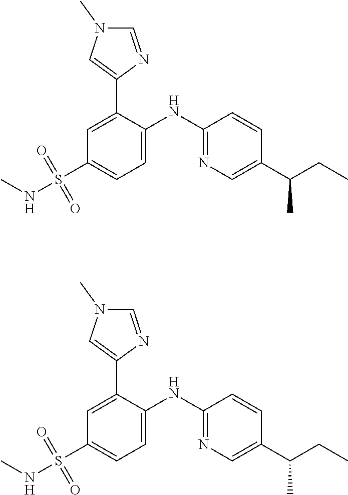

To a solution of N-[(4-methoxyphenyl)methyl]-N-methyl-3-(1-methylimidazol-4-yl)-4-[(5-sec-butyl-2-pyridyl)amino]benzenesulfonamide (140 mg, 242.47 μmol, 90%, 1 eq) in DCM (10 mL) was added TFA (2.77 g, 24.31 mmol, 1.80 mL, 100.26 eq). The mixture was stirred at 25 ºC for 1 h. The reaction mixture was quenched by addition of water (50 mL) and extracted with DCM (30 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 1/2, TLC: PE/EtOAc=1/1, R_{f}=0.26) to yield a product which was separated by chiral SFC column: DAICEL CHIRALPAK IG (250 mm*30 mm, 10 μm); mobile phase: [0.1% NH₃H₂O MeOH]; B %: 60%-60%, min) to yield Peak 1 and Peak 2. Peak 1 was concentrated under reduced pressure to yield the residue which was dissolved in MeCN (2 mL) and water (15 mL) and lyophilized to yield N-methyl-3-(1-methylimidazol-4-yl)-4-[[5-[(1R)-1-methylpropyl]-2-pyridyl]amino]benzenesulfonamide (15.03 mg, 37.62 μmol, 15.5% yield, 100.0% purity, SFC: Rₜ=2.211, ee=100%, [□]^{29.5}_{D}=+12.5 (CH₃OH, c=0.016 g/100 mL)) as a white solid. ¹H NMR (400 MHZ, CDCl₃) δ ppm 11.53 (br s, 1H), 8.70 (d, J=9.0 Hz, 1H), 8.13 (d, J=2.0 Hz, 1H), 7.95 (d, J=2.2 Hz, 1H), 7.66 (dd, J=2.1, 8.9 Hz, 1H), 7.55 (s, 1H), 7.41 (dd, J=2.2, 8.3 Hz, 1H), 7.33 (s, 1H), 6.91 (d, J=8.6 Hz, 1H), 4.20 (d, J=5.6 Hz, 1H), 3.79 (s, 3H), 2.66 (d, J=5.6 Hz, 3H), 2.62-2.55 (m, 1H), 1.64-1.60 (m, 2H), 1.25 (d, J=6.8 Hz, 3H), 0.84 (t, J=7.3 Hz, 3H); ES-LCMS m/z 400.3 [M+H]⁺. Peak 2 was concentrated under reduced pressure to yield a residue which was dissolved in MeCN (10 mL) and H₂O (20 mL) and lyophilized to yield N-methyl-3-(1-methylimidazol-4-yl)-4-[[5-[(1S)-1-methylpropyl]-2-pyridyl]amino]benzenesulfonamide (15.26 mg, 38.20 μmol, 15.7% yield, 100.0% purity) as a white solid. ¹H NMR (400 MHZ, CDCl₃) δ ppm 11.51 (br s, 1H), 8.66 (d, J=8.8 Hz, 1H), 8.10 (d, J=2.0 Hz, 1H), 7.93 (d, J=2.2 Hz, 1H), 7.63 (dd, J=2.2, 8.8 Hz, 1H), 7.53 (s, 1H), 7.38 (dd, J=2.3, 8.4 Hz, 1H), 7.31 (s, 1H), 6.88 (d, J=8.3 Hz, 1H), 4.17 (d, J=5.1 Hz, 1H), 3.77 (s, 3H), 2.63 (d, J=5.6 Hz, 3H), 2.58-2.52 (m, 1H), 1.65-1.59 (m, 2H), 1.22 (d, J=6.8 Hz, 3H), 0.81 (t, J=7.3 Hz, 3H); ES-LCMS m/z 400.3 [M+H]⁺.

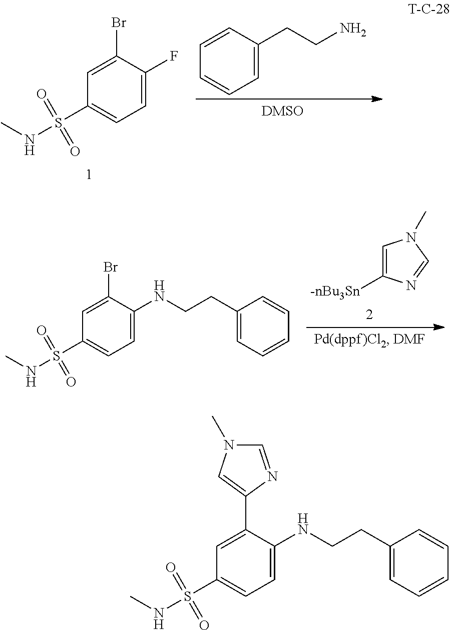

### Step 1: 3-Bromo-N-methyl-4-(2-phenylethylamino)benzenesulfonamide

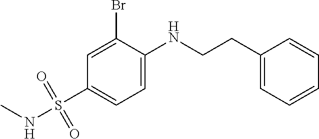 [SMILES: CNS(=O)(=O)c1ccc(NCCc2ccccc2)c(Br)c1]

To a solution of 3-bromo-4-fluoro-N-methyl-benzenesulfonamide (150 mg, 559.49 μmol, 1 eq) in DMSO (5 mL) was added 2-phenylethanamine (135.60 mg, 1.12 mmol, 140.51 μL, 2 eq). The mixture was stirred at 140° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.43) to yield 3-bromo-N-methyl-4-(2-phenylethylamino)benzenesulfonamide (200 mg, 530.77 μmol, 94.8% yield, 98.0% purity) as yellow oil. ¹H NMR (400 MHZ, CDCl₃) δ ppm 7.90 (d, J=2.2 Hz, 1H), 7.66 (dd, J=1.8, 8.7 Hz, 1H), 7.39-7.31 (m, 2H), 7.31-7.26 (m, 1H), 7.26-7.21 (m, 2H), 6.67 (d, J=8.8 Hz, 1H), 4.89 (br s, 1H), 4.29 (q, J=5.1 Hz, 1H), 3.54-3.45 (m, 2H), 2.99 (t, J=7.0 Hz, 2H), 2.64 (d, J=5.4 Hz, 3H); ES-LCMS m/z 369.1, 371.1 [M+H]⁺.

### Step 2: N-Methyl-3-(1-methylimidazol-4-yl)-4-(2-phenylethylamino)benzenesulfonamide

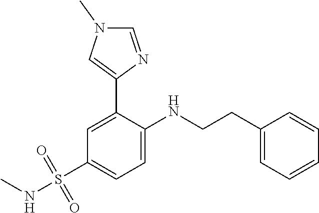 [SMILES: CNS(=O)(=O)c1ccc(NCCc2ccccc2)c(-c2cn(C)cn2)c1]

To a solution of 3-bromo-N-methyl-4-(2-phenylethylamino)benzenesulfonamide (200 mg, 530.77 μmol, 98%, 1 eq) in DMF (10 mL) was added tributyl-(1-methylimidazol-4-yl)stannane (397.97 mg, 1.06 mmol, 99%, 2 eq) and Pd(dppf)Cl₂ (38.84 mg, 53.08 μmol, 0.1 eq) was degassed and purged with N₂ for 3 times. The mixture was stirred under N₂ atmosphere at 130 ºC for 4 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which purified by flash silica gel chromatography (from PE/EtOAc=1/0 to 1/2, TLC: PE/EtOAc=1/2, R_{f}=0.43), followed by lyophilization to yield N-methyl-3-(1-methylimidazol-4-yl)-4-(2-phenylethylamino)benzenesulfonamide (133.67 mg, 353.45 μmol, 66.5% yield, 97.9% purity) as a gray solid. ¹H NMR (400 MHZ, CDCl₃) δ ppm 8.63 (br s, 1H), 7.82 (d, J=2.4 Hz, 1H), 7.58 (dd, J=2.2, 8.8 Hz, 1H), 7.45 (s, 1H), 7.35-7.28 (m, 4H), 7.26-7.20 (m, 2H), 6.71 (d, J=8.8 Hz, 1H), 4.13 (q, J=5.3 Hz, 1H), 3.76 (s, 3H), 3.55-3.49 (m, 2H), 3.03 (t, J=7.5 Hz, 2H), 2.63 (d, J=5.4 Hz, 3H); ES-LCMS m/z 371.2 [M+H]⁺.

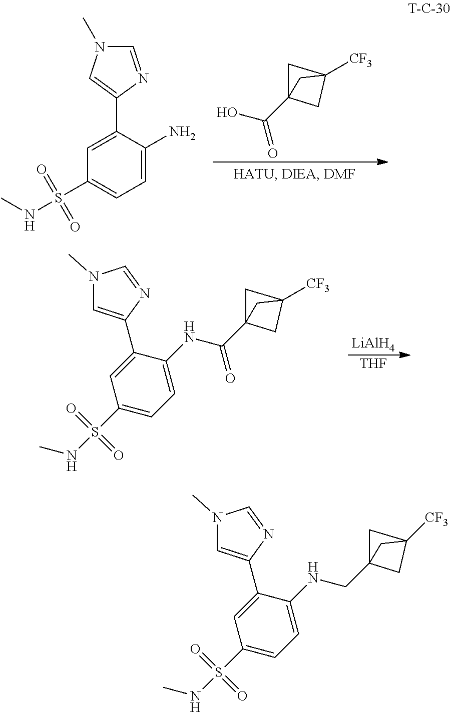

### N-[2-(1-Methylimidazol-4-yl)-4-(methylsulfamoyl)phenyl]-3-(trifluoromethyl)bicyclo[1.1.1]pentane-1-carboxamide

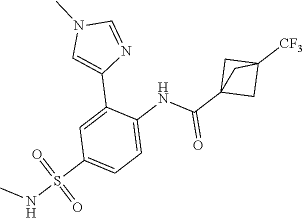 [SMILES: CNS(=O)(=O)c1ccc(NC(=O)C23CC(C(F)(F)F)(C2)C3)c(-c2cn(C)cn2)c1]

To a solution of 4-amino-N-methyl-3-(1-methylimidazol-4-yl)benzenesulfonamide (100 mg, 300.39 μmol, 80% purity, 1 eq) and 3-(trifluoromethyl)bicyclo[1.1.1]pentane-1-carboxylic acid (54.11 mg, 300.39 μmol, 1 eq) in DMF (6 mL) were added HATU (148.48 mg, 390.51 μmol, 1.3 eq) and DIPEA (77.65 mg, 600.78 μmol, 104.65 μL, 2 eq). The mixture was stirred under N₂ atmosphere at 25° C. for 16 h. The solvent was removed to yield a residue which was purified by preparative TLC (PE/EtOAc=0/1, R_{f}=0.85) to yield N-[2-(1-methylimidazol-4-yl)-4-(methylsulfamoyl)phenyl]-3-(trifluoromethyl)bicyclo[1.1.1]pentane-1-carboxamide (41 mg, 95.70 μmol, 31.9% yield, 100.0% purity) as a white solid. ¹H NMR (500 MHZ, CDCl₃) δ ppm 12.88 (s, 1H), 8.81 (d, J=8.9 Hz, 1H), 7.99 (d, J=2.1 Hz, 1H), 7.67 (dd, J=2.2, 8.8 Hz, 1H), 7.57 (s, 1H), 7.39 (d, J=1.1 Hz, 1H), 4.29 (d, J=5.3 Hz, 1H), 3.81 (s, 3H), 2.66 (d, J=5.3 Hz, 3H), 2.37 (s, 6H); ES-LCMS m/z 429.2 [M+H]⁺.

### Step 2: N-Methyl-3-(1-methylimidazol-4-yl)-4-[[3-(trifluoromethyl)-1-bicyclo[1.1.1]pentanyl]methylamino]benzenesulfonamide

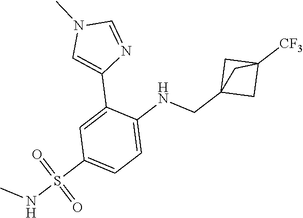 [SMILES: CNS(=O)(=O)c1ccc(NCC23CC(C(F)(F)F)(C2)C3)c(-c2cn(C)cn2)c1]

To a solution of N-[2-(1-methylimidazol-4-yl)-4-(methylsulfamoyl)phenyl]-3-(trifluoromethyl)bicyclo[1.1.1]pentane-1-carboxamide (130 mg, 300.40 μmol, 99% purity, 1 eq) in THF (5 mL) was added LiAlH₄ (34.20 mg, 901.20 μmol, 3 eq) at 25° C. and stirred for 16 h. TLC (PE/EtOAc=0/1, R_{f}=0.76) indicated the starting material was consumed completely and one new spot formed. The mixture was quenched by aq. 15% NaOH (1 mL). The mixture was filtered and the filter cake was washed with EtOAc (30 mL×2). The filtrate was concentrated to yield a residue which was purified by preparative TLC (PE/EtOAc=0/1, R_{f}=0.76) to yield a product which was dissolved in MeCN (3 mL) and H₂O (10 mL) and lyophilized to yield N-methyl-3-(1-methylimidazol-4-yl)-4-[[3-(trifluoromethyl)-1-bicyclo[1.1.1]pentanyl]methylamino]benzenesulfonamide (5.73 mg, 13.60 μmol, 4.5% yield, 98.4% purity) as a white solid. ¹H NMR (400 MHZ, CDCl₃) δ ppm 8.76 (br s, 1H), 7.83 (d, J=2.2 Hz, 1H), 7.55 (dd, J=2.1, 8.7 Hz, 1H), 7.49 (s, 1H), 7.28 (s, 1H), 6.62 (d, J=8.8 Hz, 1H), 4.31-4.15 (m, 1H), 3.83-3.69 (m, 3H), 3.39 (d, J=4.4 Hz, 2H), 2.69-2.54 (m, 3H), 2.00 (s, 6H); ES-LCMS m/z 415.2 [M+H]⁺.

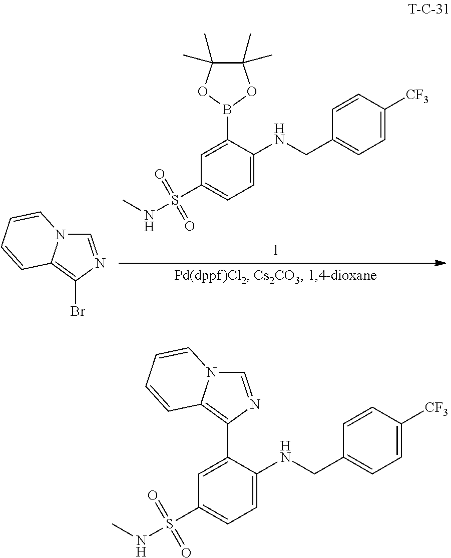 [SMILES: Brc1ncn2ccccc12 ; CNS(=O)(=O)c1ccc(NCc2ccc(C(F)(F)F)cc2)c(B2OC(C)(C)C(C)(C)O2)c1 ; CNS(=O)(=O)c1ccc(NCc2ccc(C(F)(F)F)cc2)c(-c2ncn3ccccc23)c1]

### Step 1: 3-Imidazo[1,5-a]pyridin-1-yl-N-methyl-4-[[4-(trifluoromethyl)phenyl]methylamino]benzenesulfonamide

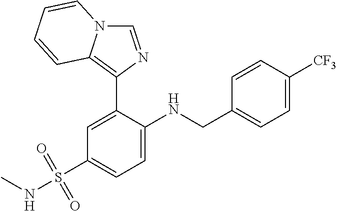 [SMILES: CNS(=O)(=O)c1ccc(NCc2ccc(C(F)(F)F)cc2)c(-c2ncn3ccccc23)c1]

To a solution of 1-bromoimidazo[1,5-a]pyridine (50 mg, 253.77 μmol, 1 eq) and N-methyl-3-(4,4,5,5-tetramethyl-1,3,2-dioxaborolan-2-yl)-4-[[4-(trifluoromethyl)phenyl]methylamino]benzenesulfonamide (159.13 mg, 304.52 μmol, 90% purity, 1.2 eq) in 1,4-dioxane (5 mL) and H₂O (0.5 mL) were added Pd(dppf)Cl₂ (18.57 mg, 25.38 μmol, 0.1 eq) and Cs₂CO₃ (165.36 mg, 507.53 μmol, 2 eq). The mixture was stirred under N₂ atmosphere at 90° C. for 16 h. The solvent was removed and the residue was treated with EtOAc (20 mL). The mixture was filtered and the filtrate was concentrated to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 47%-77%, 10 min), followed by lyophilization to yield 3-imidazo[1,5-a]pyridin-1-yl-N-methyl-4-[[4-(trifluoromethyl)phenyl]methylamino]benzenesulfonamide (55.95 mg, 118.81 μmol, 46.8% yield, 97.8% purity) as a gray solid. ¹H NMR (400 MHZ, CDCl₃) δ ppm 8.51 (t, J=5.5 Hz, 1H), 8.20 (s, 1H), 8.03 (d, J=2.0 Hz, 1H), 7.99 (d, J=7.1 Hz, 1H), 7.81 (d, J=9.3 Hz, 1H), 7.63-7.54 (m, 3H), 7.49 (d, J=8.1 Hz, 2H), 6.88 (dd, J=6.5, 9.2 Hz, 1H), 6.70 (t, J=6.7 Hz, 1H), 6.62 (d, J=8.6 Hz, 1H), 4.59 (d, J=5.4 Hz, 2H), 4.18 (br s, 1H), 2.64 (d, J=5.4 Hz, 3H); ES-LCMS m z 460.9 [M+H]⁺.

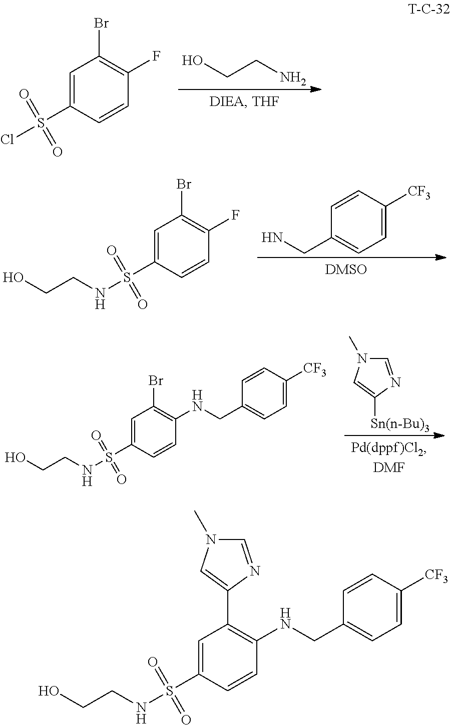

### Step 1: 3-Bromo-4-fluoro-N-(2-hydroxyethyl)benzenesulfonamide

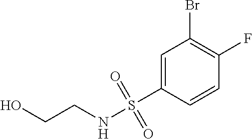 [SMILES: O=S(=O)(NCCO)c1ccc(F)c(Br)c1]

To a solution of 3-bromo-4-fluoro-benzenesulfonyl chloride (400 mg, 1.46 mmol, 1 eq) in THF (6 mL) was added DIEA (378.03 mg, 2.92 mmol, 509.48 μL, 2 eq) and 2-aminoethanol (134.00 mg, 2.19 mmol, 132.67 μL, 1.5 eq). The mixture was stirred at 20° C. for 1 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield 3-bromo-4-fluoro-N-(2-hydroxyethyl)benzenesulfonamide (400 mg, crude) as white oil, which was used in the next step without further purification. ¹H NMR (400 MHZ, CDCl₃) δ ppm 10.72 (s, 1H), 8.14 (dd, J=2.3, 6.2 Hz, 1H), 7.88 (ddd, J=2.2, 4.4, 8.6 Hz, 1H), 7.26-7.21 (m, 1H), 6.03 (s, 1H), 3.14-3.10 (m, 4H).

### Step 2: 3-Bromo-N-(2-hydroxyethyl)-4-[[4-(trifluoromethyl)phenyl]methylamino]benzenesulfonamide

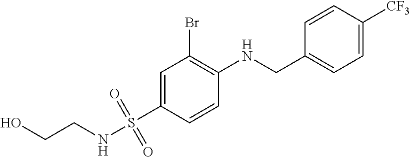 [SMILES: O=S(=O)(NCCO)c1ccc(NCc2ccc(C(F)(F)F)cc2)c(Br)c1]

To a solution of 3-bromo-4-fluoro-N-(2-hydroxyethyl)benzenesulfonamide (400 mg, 1.34 mmol, N/A purity, 1 eq) in DMSO (3 mL) was added [4-(trifluoromethyl)phenyl]methanamine (470.00 mg, 2.68 mmol, 382.11 μL, 2 eq). The mixture was stirred at 140° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 0/1, TLC: PE/EtOAc=0/1, R_{f} 0.40) to yield 3-bromo-N-(2-hydroxyethyl)-4-[[4-(trifluoromethyl)phenyl]methylamino]benzenesulfonamide (580 mg, 959.69 μmol, 71.5% yield, 75.0% purity) as yellow oil. ¹H NMR (400 MHZ, CDCl₃) δ ppm 7.98 (d, J=2.0 Hz, 1H), 7.65 (d, J=8.1 Hz, 2H), 7.46 (d, J=8.1 Hz, 2H), 6.54 (d, J=8.6 Hz, 1H), 5.40-5.31 (m, 1H), 4.77 (t, J=6.1 Hz, 1H), 4.57 (d, J=5.6 Hz, 2H), 4.13 (q, J=7.3 Hz, 1H), 3.74-3.71 (m, 2H), 3.13-3.05 (m, 2H), ES-LCMS m/z 453.0, 455.0 [M+H]⁺.

### Step 3: N-(2-Hydroxyethyl)-3-(1-methylimidazol-4-yl)-4-[[4-(trifluoromethyl)phenyl]methylamino]benzenesulfonamide

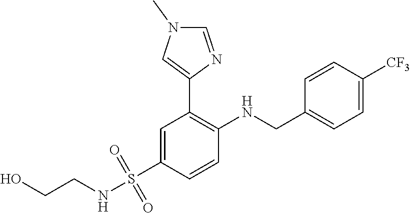 [SMILES: Cn1cnc(-c2cc(S(=O)(=O)NCCO)ccc2NCc2ccc(C(F)(F)F)cc2)c1]

A mixture of 3-bromo-N-(2-hydroxyethyl)-4-[[4-(trifluoromethyl)phenyl]methylamino]benzenesulfonamide (580 mg, 959.69 μmol, 75% purity, 1 eq), tributyl-(1-methylimidazol-4-yl)stannane (890.46 mg, 2.40 mmol, 2.5 eq), Pd(dppf)Cl₂ (70.22 mg, 95.97 μmol, 0.1 eq), tributyl-(1-methylimidazol-4-yl)stannane (890.46 mg, 2.40 mmol, 2.5 eq) in DMF (6 mL) was degassed and purged with N₂ for 3 times and the mixture was stirred under N₂ atmosphere at 130° C. for 8 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.49) and lyophilized to yield N-(2-hydroxyethyl)-3-(1-methylimidazol-4-yl)-4-[[4-(trifluoromethyl)phenyl]methylamino]benzenesulfonamide (274.08 mg, 583.18 μmol, 60.8% yield, 96.7% purity) as white solid. ¹H NMR (400 MHZ, CDCl₃) δ ppm 9.28-9.20 (m, 1H), 7.89 (d, J=2.3 Hz, 1H), 7.60 (d, J=7.8 Hz, 2H), 7.52-7.47 (m, 4H), 7.32 (d, J=1.2 Hz, 1H), 6.54 (d, J=8.6 Hz, 1H), 4.76 (t, J=6.3 Hz, 1H), 4.60 (d, J=5.5 Hz, 2H), 3.78 (s, 3H), 3.69 (t, J=5.1 Hz, 2H), 3.13-3.02 (m, 2H). ES-LCMS m/z 455.2 [M+H]⁺.

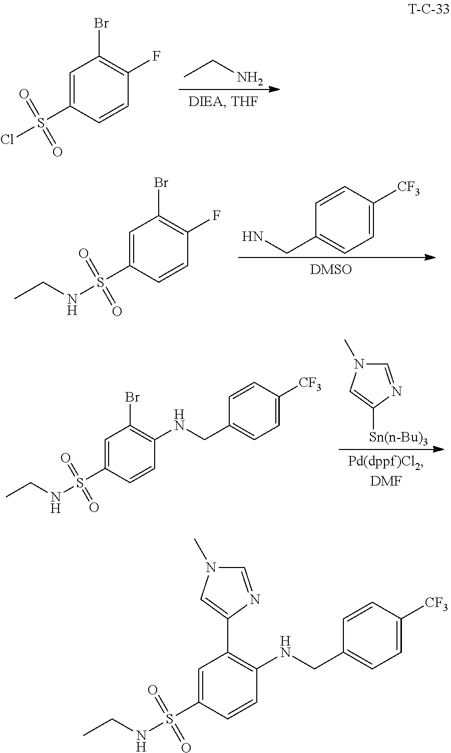

### Step 1: 3-Bromo-N-ethyl-4-fluoro-benzenesulfonamide

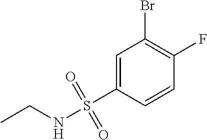

To a solution of ethanamine (238.51 mg, 2.92 mmol, 346.18 μL, 2 eq, HCl) in THF (6 mL) was added DIEA (472.54 mg, 3.66 mmol, 636.85 μL, 2.5 eq) and 3-bromo-4-fluoro-benzenesulfonyl chloride (400 mg, 1.46 mmol, 1 eq). The mixture was stirred at 20° C. for 2 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield 3-bromo-N-ethyl-4-fluoro-benzenesulfonamide (400 mg, 1.42 mmol, 96.9% yield, N/A purity) as a white oil, which was used in the next step without further purification. ¹H NMR (400 MHZ, CDCl₃) δ ppm 8.11 (dd, J=2.2, 6.4 Hz, 1H), 7.83 (ddd, J=2.2, 4.3, 8.6 Hz, 1H), 7.27-7.23 (m, 1H), 4.75 (s, 1H), 3.07-3.00 (m, 2H), 1.14 (t, J=7.2 Hz, 3H).

### Step 2: 3-Bromo-N-ethyl-4-[[4-(trifluoromethyl)phenyl]methylamino]benzenesulfonamide

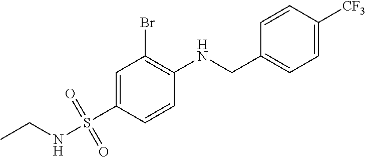 [SMILES: CCNS(=O)(=O)c1ccc(NCc2ccc(C(F)(F)F)cc2)c(Br)c1]

To a solution of 3-bromo-N-ethyl-4-fluoro-benzenesulfonamide (400 mg, 1.42 mmol, N/A purity, 1 eq) in DMSO (4 mL) was added [4-(trifluoromethyl)phenyl]methanamine (496.65 mg, 2.84 mmol, 403.78 μL, 2 eq). The mixture was stirred at 140° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 1/1, TLC: PE/EtOAc=1/1, R_{f}=0.40) to yield a 3-bromo-N-ethyl-4-[[4-(trifluoromethyl)phenyl]methylamino]benzenesulfonamide (217 mg, 397.01 μmol, 28.0% yield, 80.0% purity) as yellow oil. ¹H NMR (400 MHZ, CDCl₃) δ ppm 7.97 (d, J=2.2 Hz, 1H), 7.64 (d, J=8.1 Hz, 2H), 7.46 (d, J=8.1 Hz, 2H), 6.54 (d, J=8.6 Hz, 1H), 5.41-5.27 (m, 1H), 4.56 (d, J=5.9 Hz, 2H), 3.00 (dd, J=6.4, 7.1 Hz, 2H), 2.91 (s, 2H), 1.14-1.11 (m, 3H), ES-LCMS m/z 437.0, 438.9 [M+H]⁺.

### Step 3: N-Ethyl-3-(1-methylimidazol-4-yl)-4-[[4-(trifluoromethyl)phenyl]methylamino]benzenesulfonamide

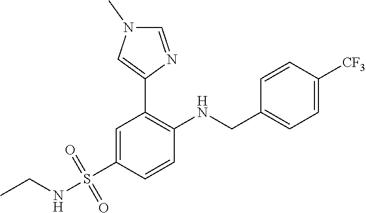 [SMILES: CCNS(=O)(=O)c1ccc(NCc2ccc(C(F)(F)F)cc2)c(-c2cn(C)cn2)c1]

A mixture of 3-bromo-N-ethyl-4-[[4-(trifluoromethyl)phenyl]methylamino]benzenesulfonamide (217 mg, 397.01 μmol, 80% purity, 1 eq), tributyl-(1-methylimidazol-4-yl)stannane (372.09 mg, 992.51 μmol, 99% purity, 2.5 eq), Pd(dppf)Cl₂ (29.05 mg, 39.70 μmol, 0.1 eq) in DMF (5 mL) was degassed and purged with N₂ for 3 times and the mixture was stirred under N₂ atmosphere at 130° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 1/1, TLC: PE/EtOAc=1/1, R_{f}=0.20) and by preparative HPLC (column: Agela DuraShell C18 150×25 mm×5 μm; mobile phase: [water (0.05% NH₃H₂O+10 mM NH₄HCO₃)-ACN]; B %: 43%-73%, 10 min) and lyophilized to yield N-ethyl-3-(1-methylimidazol-4-yl)-4-[[4-(trifluoromethyl)phenyl]methylamino]benzenesulfonamide (30.89 mg, 70.45 μmol, 17.6% yield, 100.0% purity) as a white solid. ¹H NMR (400 MHZ, CDCl₃) δ ppm 9.23 (t, J=5.7 Hz, 1H), 7.88 (d, J=2.2 Hz, 1H), 7.59 (d, J=8.1 Hz, 2H), 7.49 (d, J=6.4 Hz, 4H), 7.32 (s, 1H), 6.54 (d, J=8.8 Hz, 1H), 4.60 (d, J=5.6 Hz, 2H), 4.11 (t, J=6.2 Hz, 1H), 3.78 (s, 3H), 2.98 (q, J=7.0 Hz, 2H), 1.10 (t, J=7.2 Hz, 3H), ES-LCMS m/z 439.2 [M+H]⁺.

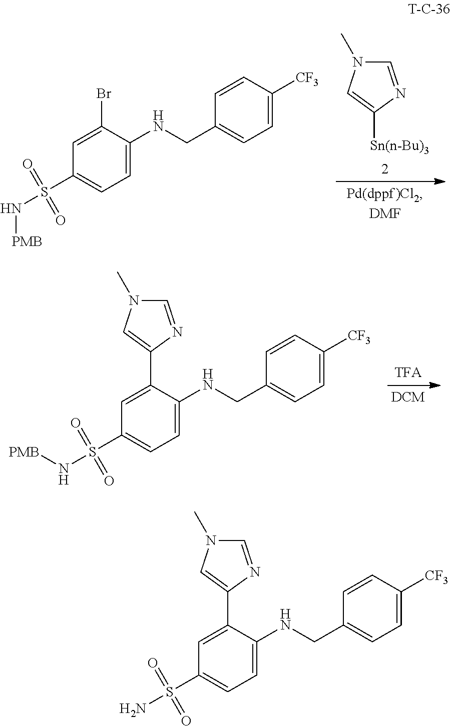

### Step 1: N-[(4-Methoxyphenyl)methyl]-3-(1-methylimidazol-4-yl)-4-[[4-(trifluoromethyl)phenyl]methylamino]benzenesulfonamide

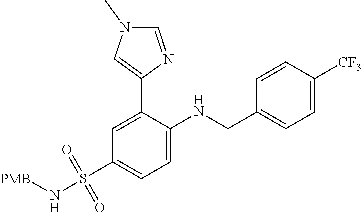

To a stirred solution of 3-bromo-N-[(4-methoxyphenyl)methyl]-4-[[4-(trifluoromethyl)phenyl]methylamino]benzenesulfonamide (1 g, 1.70 mmol, 90%, 1 eq) and tributyl-(1-methylimidazol-4-yl)stannane (832.09 mg, 2.04 mmol, 91%, 1.2 eq) in DMF (10 mL) was added Pd(dppf)Cl₂ (124.40 mg, 170.01 μmol, 0.1 eq). The reaction mixture bubbled with N₂ for 1 min and stirred under microwave at 130° C. for 1.5 h. The reaction mixture was quenched by addition of water (100 mL) and extracted with EtOAc (50 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 1/1, TLC: PE/EtOAc=1/1, R_{f}=0.50) to yield N-[(4-methoxyphenyl)methyl]-3-(1-methylimidazol-4-yl)-4-[[4-(trifluoromethyl)phenyl]methylamino]benzenesulfonamide (695 mg, 1.05 mmol, 61.6% yield, 80.0% purity) as a yellow solid. ¹H NMR (400 MHZ, CDCl₃) δ ppm 9.27 (t, J=5.3 Hz, 1H), 7.88 (s, 1H), 7.61 (d, J=7.8 Hz, 2H), 7.53-7.48 (m, 4H), 7.43 (d, J=7.8 Hz, 1H), 7.30 (s, 1H), 7.12 (d, J=8.3 Hz, 2H), 6.80 (d, J=8.1 Hz, 2H), 6.55 (d, J=8.8 Hz, 1H), 4.62 (d, J=5.6 Hz, 2H), 4.03 (d, J=6.1 Hz, 2H), 3.78 (s, 3H), 3.77 (s, 3H); ES-LCMS m/z 531.1 [M+H]⁺.

### Step 2: 3-(1-Methylimidazol-4-yl)-4-[[4-(trifluoromethyl)phenyl]methylamino]benzenesulfonamide

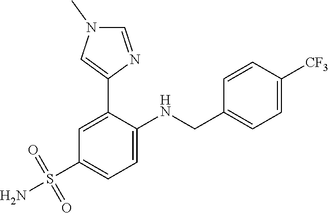 [SMILES: Cn1cnc(-c2cc(S(N)(=O)=O)ccc2NCc2ccc(C(F)(F)F)cc2)c1]

To a solution of N-[(4-methoxyphenyl)methyl]-3-(1-methylimidazol-4-yl)-4-[[4-(trifluoromethyl)phenyl]methylamino]benzenesulfonamide (695 mg, 1.31 mmol, 1 eq) in DCM (10 mL) was added TFA (15.40 g, 135.06 mmol, 10.00 mL, 103.10 eq). The mixture was stirred at 25° C. for 48 h. The reaction mixture was quenched by addition of aq. NaHCO₃ (100 mL) and extracted with EtOAc (50 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 1/1, TLC: PE/EtOAc=1/1, R_{f}=0.20) to yield 3-(1-methylimidazol-4-yl)-4-[[4-(trifluoromethyl)phenyl]methylamino]benzenesulfonamide (162.21 mg, 375.79 μmol, 29.0% yield, 96.9% purity) as a white solid. ¹H NMR (500 MHz, DMSO-d₆) δ ppm 9.22 (t, J=6.3 Hz, 1H), 7.88 (d, J=2.1 Hz, 1H), 7.80 (s, 1H), 7.69 (d, J=8.1 Hz, 2H), 7.64 (s, 1H), 7.54 (d, J=8.1 Hz, 2H), 7.37 (dd, J=2.1, 8.7 Hz, 1H), 6.94 (s, 2H), 6.58 (d, J=8.7 Hz, 1H), 4.64 (d, J=5.6 Hz, 2H), 3.75 (s, 3H); ES-LCMS m/z 411.1 [M+H]⁺.

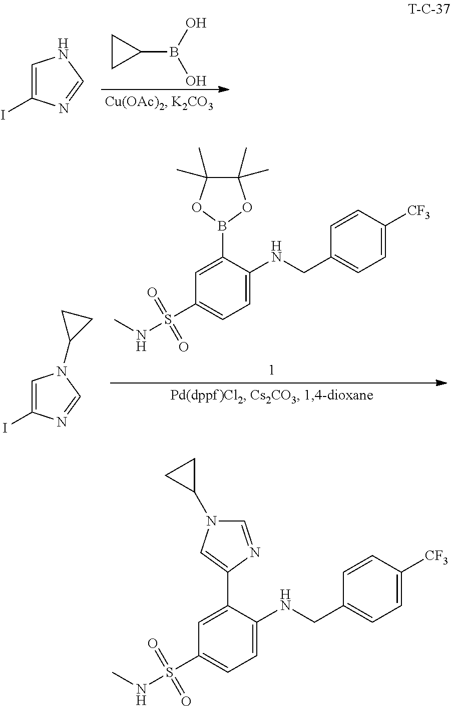

### Step 1: 1-Cyclopropyl-4-iodo-imidazole

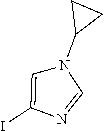

To a solution of 4-iodo-1H-imidazole (2.7 g, 13.92 mmol, 1 eq) in 1,2-dichloroethane (25 mL) was added 2-(2-pyridyl)pyridine (2.17 g, 13.92 mmol, 1 eq), Cu(OAc)₂ (2.53 g, 13.92 mmol, 1 eq), K₂CO₃ (3.85 g, 27.84 mmol, 2 eq) and cyclopropylboronic acid (2.03 g, 23.66 mmol, 1.7 eq). The mixture was stirred under N₂ atmosphere at 50° C. for 16 h. The reaction mixture was filtered through a pad of celite and the filtrate was concentrated to yield a residue which was purified by flash silica gel chromatography (from PE/DCM=1/0 to 1/2, TLC: PE/DCM=1/2, R_{f}=0.60) to yield 1-cyclopropyl-4-iodo-imidazole (1.3 g, 2.22 mmol, 15.9% yield, 40.0% purity) as a yellow solid. ¹H NMR (400 MHZ, CDCl₃) δ ppm 7.34 (d, J=1.0 Hz, 1H), 7.31 (d, J=1.0 Hz, 1H), 3.33 (tt, J=3.7, 7.2 Hz, 1H), 1.04-0.90 (m, 4H); ES-LCMS m/z 235.1 [M+H]⁺.

### Step 2: 3-(1-Cyclopropylimidazol-4-yl)-N-methyl-4-[[4-(trifluoromethyl)phenyl]methylamino]benzenesulfonamide

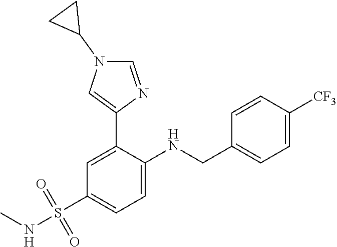 [SMILES: CNS(=O)(=O)c1ccc(NCc2ccc(C(F)(F)F)cc2)c(-c2cn(C3CC3)cn2)c1]

To a solution of N-methyl-3-(4,4,5,5-tetramethyl-1,3,2-dioxaborolan-2-yl)-4-[[4-(trifluoromethyl)phenyl]methylamino]benzenesulfonamide (150 mg, 287.04 μmol, 90%, 1 eq) and 1-cyclopropyl-4-iodo-imidazole (201.54 mg, 344.45 μmol, 40%, 1.2 eq) in 1,4-dioxane (5 mL) and H₂O (1.5 mL) was added Cs₂CO₃ (187.05 mg, 574.09 μmol, 2 eq) and Pd(dppf)Cl₂ (21.00 mg, 28.70 μmol, 0.1 eq). The mixture was stirred under N₂ atmosphere at 100° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by preparative HPLC column: Agela DuraShell C18 150*25 mm*5 μm; mobile phase: [water (0.05% NH₃·H₂O+10 mM NH₄HCO₃)-ACN]; B %: 47%-77%, 10 min), followed by lyophilization to yield 3-(1-cyclopropylimidazol-4-yl)-N-methyl-4-[[4-(trifluoromethyl)phenyl]methylamino]benzenesulfonamide (63.84 mg, 138.40 μmol, 48.2% yield, 97.6% purity) as a white solid. ¹H NMR (400 MHZ, CDCl₃) δ ppm 9.21 (t, J=5.1 Hz, 1H), 7.87-7.83 (m, 1H), 7.57 (d, J=8.3 Hz, 3H), 7.46 (d, J=7.3 Hz, 3H), 7.38 (s, 1H), 6.51 (d, J=8.8 Hz, 1H), 4.57 (d, J=5.6 Hz, 2H), 4.11 (q, J=5.2 Hz, 1H), 3.44-3.37 (m, 1H), 2.60 (d, J=5.6 Hz, 3H), 1.10-0.99 (m, 4H); ES-LCMS m/z 451.2 [M+H]⁺.

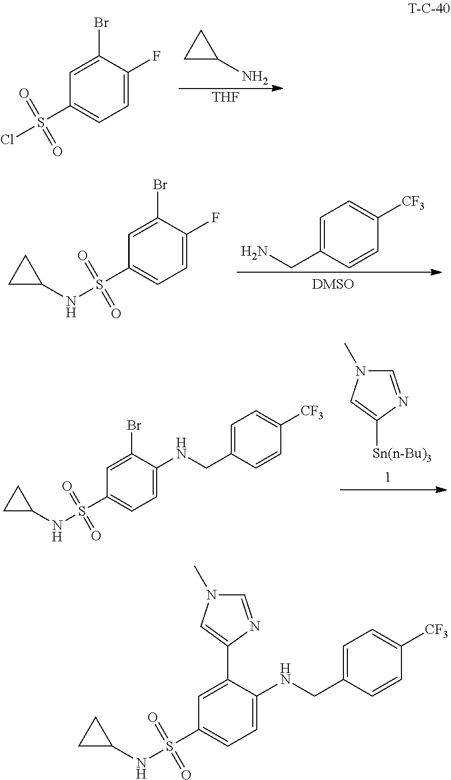

### Step 1: 3-Bromo-N-cyclopropyl-4-fluoro-benzenesulfonamide

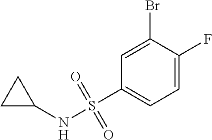 [SMILES: O=S(=O)(NC1CC1)c1ccc(F)c(Br)c1]

To a stirred solution of 3-bromo-4-fluoro-benzenesulfonyl chloride (700 mg, 2.56 mmol, 1 eq) in THF (12 mL) was added cyclopropanamine (438.37 mg, 7.68 mmol, 532.01 μL, 3 eq) at −60° C. The reaction mixture was stirred under N₂ atmosphere −60° C. for 1 h. TLC (PE/EtOAc=10/1, R_{f}=0.16) showed start material was consumed completely and one new spot was detected. The reaction mixture was quenched by addition 1 N HCl (8 mL) at −60° C. and extracted with EtOAc (30 mL×3). The combined organic layers were washed with brine (30 mL), dried over Na₂SO₄, filtered and concentrated under reduced pressure to yield 3-bromo-N-cyclopropyl-4-fluoro-benzenesulfonamide (800 mg, 2.50 mmol, 97.8% yield, 92.0% purity) as a white solid. ¹H NMR (400 MHZ, CDCl₃) δ ppm 8.12 (dd, J=2.3, 6.2 Hz, 1H), 7.86-7.82 (m, 1H), 7.29-7.25 (m, 1H), 4.85 (s, 1H), 2.31-2.24 (m, 1H), 0.64-0.60 (m, 4H); ES-LCMS m/z 294.1, 296.1 [M+H]⁺.

### Step 2: 3-Bromo-N-cyclopropyl-4-[[4-(trifluoromethyl)phenyl]methylamino]benzenesulfonamide

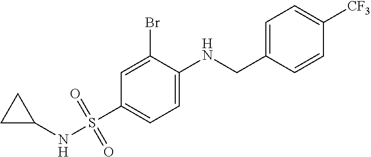 [SMILES: O=S(=O)(NC1CC1)c1ccc(NCc2ccc(C(F)(F)F)cc2)c(Br)c1]

To a stirred solution of 3-bromo-N-cyclopropyl-4-fluoro-benzenesulfonamide (200 mg, 625.55 μmol, 92.0% purity, 1 eq) in DMSO (5 mL) was added [4-(trifluoromethyl)phenyl]methanamine (219.13 mg, 1.25 mmol, 178.16 μL, 2 eq). The reaction mixture was stirred under N₂ atmosphere at 140° C. for 12 h. The reaction mixture was diluted with H₂O (20 mL) and extracted with EtOAc (40 mL×3). The combined organic layers were washed with brine (30 mL), dried over Na₂SO₄, filtered and concentrated under reduced pressure to yield a residue which was purified by flash silica gel chromatography (from PE/EtOAc=100/1 to 5/1, TLC: PE/EtOAc=1/1, R_{f}=0.43) to yield 3-bromo-N-cyclopropyl-4-[[4-(trifluoromethyl)phenyl]methylamino]benzenesulfonamide (280 mg, 573.36 μmol, 91.7% yield, 92.0% purity) as a white solid. ¹H NMR (400 MHZ, DMSO-d₆) δ ppm 7.78 (d, J=2.2 Hz, 1H), 7.70 (d, J=8.1 Hz, 2H), 7.62 (d, J=2.7 Hz, 1H), 7.53 (d, J=8.1 Hz, 2H), 7.46 (dd, J=2.0, 8.6 Hz, 1H), 6.92 (t, J=6.1 Hz, 1H), 6.61 (d, J=8.8 Hz, 1H), 4.60 (d, J=5.9 Hz, 2H), 2.04-1.97 (m, 1H), 0.48-0.41 (m, 2H), 0.37-0.30 (m, 2H); ES-LCMS m/z 449.1, 451.1 [M+H]⁺.

### Step 3: N-Cyclopropyl-3-(1-methylimidazol-4-yl)-4-[[4-(trifluoromethyl)phenyl]methylamino]benzenesulfonamide

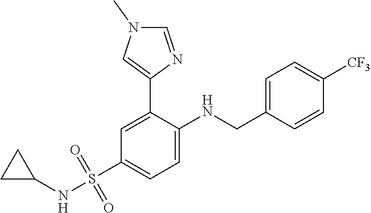 [SMILES: Cn1cnc(-c2cc(S(=O)(=O)NC3CC3)ccc2NCc2ccc(C(F)(F)F)cc2)c1]

To a stirred solution of 3-bromo-N-cyclopropyl-4-[[4-(trifluoromethyl)phenyl]methylamino]benzenesulfonamide (150 mg, 307.15 μmol, 92.0% purity, 1 eq) and tributyl-(1-methylimidazol-4-yl)stannane (230.30 mg, 614.31 μmol, 99.0% purity, 2 eq) in DMF (4 mL) was added Pd(dppf)Cl₂ (22.47 mg, 30.72 μmol, 0.1 eq). The reaction mixture was stirred under N₂ atmosphere at 140° C. for 5 h. The reaction mixture was diluted with H₂O (20 mL) and extracted with EtOAc (30 mL×3). The combined organic layers were washed with brine (20 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 50%-80%, 10 min) to yield N-cyclopropyl-3-(1-methylimidazol-4-yl)-4-[[4-(trifluoromethyl)phenyl]methylamino]benzenesulfonamide (80.72 mg, 177.45 μmol, 57.8% yield, 99.0% purity) as an off-white solid. ¹H NMR (400 MHZ, DMSO-d₆) δ ppm 9.30 (t, J=5.9 Hz, 1H), 7.84 (d, J=2.2 Hz, 1H), 7.81 (s, 1H), 7.74-7.66 (m, 3H), 7.56 (d, J=8.1 Hz, 2H), 7.48 (s, 1H), 7.37 (dd, J=2.1, 8.7 Hz, 1H), 6.64 (d, J=8.8 Hz, 1H), 4.65 (d, J=5.9 Hz, 2H), 3.75 (s, 3H), 2.03 (s, 1H), 0.48-0.40 (m, 2H), 0.38-0.31 (m, 2H); ES-LCMS m/z 451.2 [M+H]⁺.

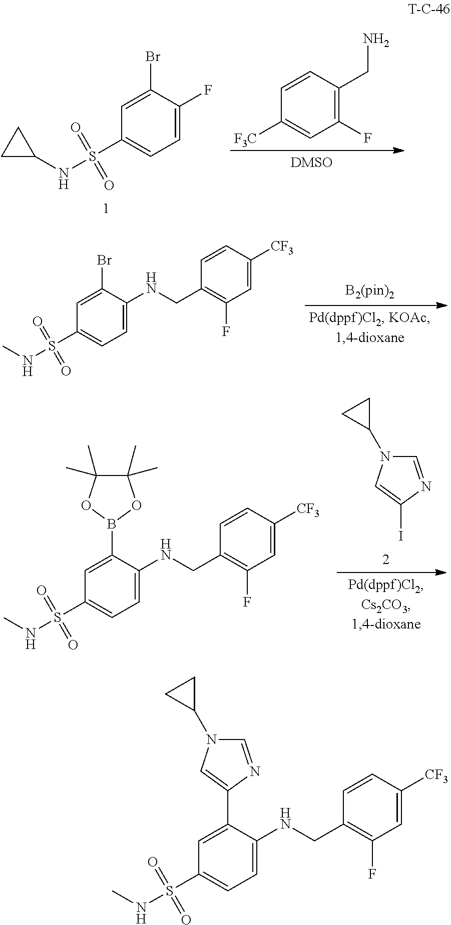 [SMILES: O=S(=O)(NC1CC1)c1ccc(F)c(Br)c1 ; NCc1ccc(C(F)(F)F)cc1F ; CNS(=O)(=O)c1ccc(NCc2ccc(C(F)(F)F)cc2F)c(Br)c1 ; CNS(=O)(=O)c1ccc(NCc2ccc(C(F)(F)F)cc2F)c(B2OC(C)(C)C(C)(C)O2)c1 ; Ic1cn(C2CC2)cn1 ; CNS(=O)(=O)c1ccc(NCc2ccc(C(F)(F)F)cc2F)c(-c2cn(C3CC3)cn2)c1]

### Step 1: 3-Bromo-4-[[2-fluoro-4-(trifluoromethyl)phenyl]methylamino]-N-methyl-benzenesulfonamide

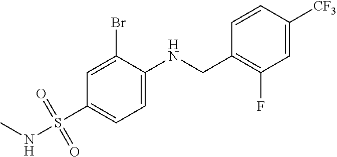 [SMILES: CNS(=O)(=O)c1ccc(NCc2ccc(C(F)(F)F)cc2F)c(Br)c1]

To a solution of [2-fluoro-4-(trifluoromethyl)phenyl]methanamine (200 mg, 1.04 mmol, 1 eq) in DMSO (10 mL) was added 3-bromo-4-fluoro-N-methyl-benzenesulfonamide (462.7 mg, 1.6 mmol, 90%, 1.5 eq). The mixture was stirred at 140° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.46) to yield a 3-bromo-4-[[2-fluoro-4-(trifluoromethyl)phenyl]methylamino]-N-methyl-benzenesulfonamide (380 mg, 790.8 μmol, 76.4% yield, 91.8% purity) as a colorless solid. ¹H NMR (400 MHZ, CDCl₃) δ ppm 7.97 (d, J=1.7 Hz, 1H), 7.62 (dd, J=1.6, 8.7 Hz, 1H), 7.44-7.37 (m, 3H), 6.57 (d, J=8.6 Hz, 1H), 5.34 (t, J=5.6 Hz, 1H), 4.62 (d, J=6.1 Hz, 2H), 4.24 (d, J=5.1 Hz, 1H), 2.65 (d, J=5.4 Hz, 3H); ES-LCMS m/z 441.0, 443.0 [M+H]⁺.

### Step 2: 4-[[2-Fluoro-4-(trifluoromethyl)phenyl]methylamino]-N-methyl-3-(4,4,5,5-tetramethyl-1,3,2-dioxaborolan-2-yl)benzenesulfonamide

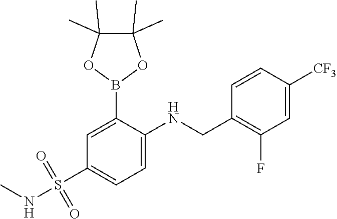 [SMILES: CNS(=O)(=O)c1ccc(NCc2ccc(C(F)(F)F)cc2F)c(B2OC(C)(C)C(C)(C)O2)c1]

To a solution of 3-bromo-4-[[2-fluoro-4-(trifluoromethyl)phenyl]methylamino]-N-methyl-benzenesulfonamide (260 mg, 525.5 μmol, 89.2%, 1 eq) and 4,4,5,5-tetramethyl-2-(4,4,5,5-tetramethyl-1,3,2-dioxaborolan-2-yl)-1,3,2-dioxaborolane (400.33 mg, 1.58 mmol, 3 eq) in 1,4-dioxane (5 mL) was added Pd(dppf)Cl₂ (38.45 mg, 52.55 μmol, 0.1 eq) and KOAc (103.15 mg, 1.05 mmol, 2 eq). The mixture was stirred at 100° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.43) to yield 4-[[2-fluoro-4-(trifluoromethyl)phenyl]methylamino]-N-methyl-3-(4,4,5,5-tetramethyl-1,3,2-dioxaborolan-2-yl)benzenesulfonamide (150 mg, 246.86 μmol, 47.0% yield, 80.4% purity) as colorless oil. ¹H NMR (400 MHz, CDCl₃) δ ppm 8.11 (d, J=2.2 Hz, 1H), 7.67 (dd, J=2.3, 8.7 Hz, 1H), 7.44-7.38 (m, 1H), 7.38-7.32 (m, 2H), 6.86 (t, J=6.1 Hz, 1H), 6.44 (d, J=9.0 Hz, 1H), 4.55 (d, J=5.9 Hz, 2H), 3.90 (br s, 1H), 2.59 (d, J=5.4 Hz, 3H), 1.33 (s, 12H); ES-LCMS m/z 489.2 [M+H]⁺.

### Step 3: 3-(1-Cyclopropylimidazol-4-yl)-4-[[2-fluoro-4-(trifluoromethyl)phenylmethylamino]-N-methyl-benzenesulfonamide

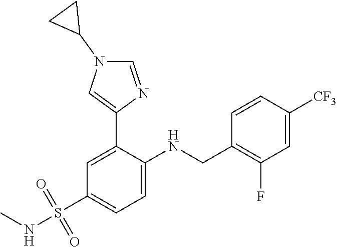 [SMILES: CNS(=O)(=O)c1ccc(NCc2ccc(C(F)(F)F)cc2F)c(-c2cn(C3CC3)cn2)c1]

To a solution of 4-[[2-fluoro-4-(trifluoromethyl)phenyl]methylamino]-N-methyl-3-(4,4,5,5-tetramethyl-1,3,2-dioxaborolan-2-yl)benzenesulfonamide (150 mg, 246.86 μmol, 80.4%, 1.16 eq) and 4-[[2-fluoro-4-(trifluoromethyl)phenyl]methylamino]-N-methyl-3-(4,4,5,5-tetramethyl-1,3,2-dioxaborolan-2-yl)benzenesulfonamide (149.29 mg, 255.15 μmol, 40%, 1.2 eq) in H₂O (1 mL) and 1,4-dioxane (5 mL) was added Cs₂CO₃ (138.55 mg, 425.25 μmol, 2 eq) and Pd(dppf)Cl₂ (15.56 mg, 21.26 μmol, 0.1 eq). The mixture was stirred at 100° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to give a residue which was purified by preparative HPLC (column: Agela DuraShell C18 150*25 mm*5 μm; mobile phase: [water (0.05% NH₃·H₂O+10 mM NH₄HCO₃)-ACN]; B %: 52%-82%, 10 min), followed by lyophilization to yield 3-(1-cyclopropylimidazol-4-yl)-4-[[2-fluoro-4-(trifluoromethyl)phenyl]methylamino]-N-methyl-benzenesulfonamide (13.56 mg, 28.95 μmol, 13.6% yield, 100.0% purity) as a white solid. ¹H NMR (400 MHZ, CDCl₃) δ ppm 9.24 (t, J=5.6 Hz, 1H), 7.89 (d, J=2.0 Hz, 1H), 7.61 (s, 1H), 7.55-7.47 (m, 2H), 7.42 (s, 1H), 7.35 (d, J=8.8 Hz, 2H), 6.55 (d, J=8.8 Hz, 1H), 4.64 (d, J=5.6 Hz, 2H), 4.25 (q, J=5.1 Hz, 1H), 3.47-3.39 (m, 1H), 2.63 (d, J=5.6 Hz, 3H), 1.11-1.02 (m, 4H); ES-LCMS m/z 469.2 [M+H]⁺.

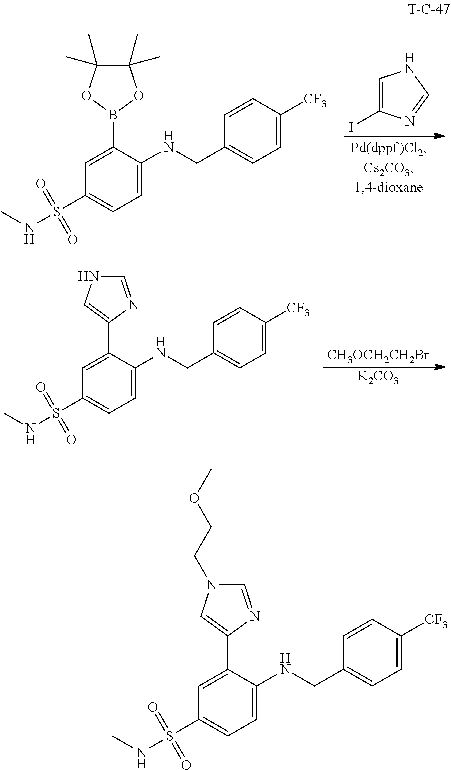

### Step 1: 3-(1H-Imidazol-4-yl)-N-methyl-4-[[4-(trifluoromethyl)phenyl]methylamino]benzenesulfonamide

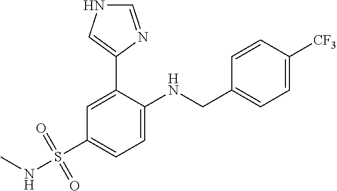 [SMILES: CNS(=O)(=O)c1ccc(NCc2ccc(C(F)(F)F)cc2)c(-c2c[nH]cn2)c1]

To a solution of N-methyl-3-(4,4,5,5-tetramethyl-1,3,2-dioxaborolan-2-yl)-4-[[4-(trifluoromethyl)phenyl]methylamino]benzenesulfonamide (500 mg, 956.81 μmol, 90%, 1 eq) and 4-iodo-1H-imidazole (222.71 mg, 1.15 mmol, 1.2 eq) in 1,4-dioxane (10 mL) and H₂O (2 mL) was added Cs₂CO₃ (623.49 mg, 1.91 mmol, 2 eq) and Pd(dppf)Cl₂ (70.01 mg, 95.68 μmol, 0.1 eq). The mixture was stirred under N₂ atmosphere at 100° C. for 12 h. The reaction mixture was quenched by addition of water (30 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 1/2, TLC: PE/EtOAc=1/1, R_{f}=0.44) to yield 3-(1H-imidazol-4-yl)-N-methyl-4-[[4-(trifluoromethyl)phenyl]methylamino]benzenesulfonamide (300 mg, 642.60 μmol, 67.1% yield, 87.9% purity) as a white solid. ¹H NMR (400 MHZ, CDCl₃) δ ppm 9.19 (br s, 1H), 7.92 (d, J=2.2 Hz, 1H), 7.74 (s, 1H), 7.60 (d, J=8.1 Hz, 2H), 7.54-7.49 (m, 3H), 7.46 (s, 1H), 6.56 (d, J=8.6 Hz, 1H), 4.61 (br s, 2H), 4.24 (d, J=5.6 Hz, 1H), 2.63 (d, J=5.6 Hz, 3H); ES-LCMS m/z 411.2 [M+H]⁺.

### Step 2: 3-[1-(2-Methoxyethyl)imidazol-4-yl]-N-methyl-4-[[4-(trifluoromethyl)phenyl]methylamino]benzenesulfonamide

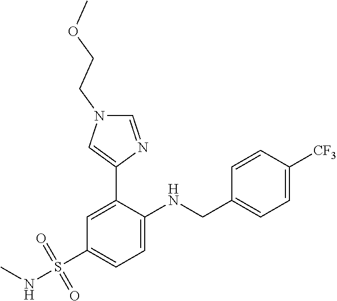 [SMILES: CNS(=O)(=O)c1ccc(NCc2ccc(C(F)(F)F)cc2)c(-c2cn(CCOC)cn2)c1]

To a solution of 3-(1H-imidazol-4-yl)-N-methyl-4-[[4-(trifluoromethyl)phenyl]methylamino]benzenesulfonamide (50 mg, 107.10 μmol, 87.9%, 1 eq) in DMF (5 mL) was added K₂CO₃ (29.60 mg, 214.2 μmol, 2 eq) and 1-bromo-2-methoxy-ethane (17.86 mg, 128.52 μmol, 12.07 μL, 1.2 eq). The mixture was stirred at 25° C. for 12 h. The reaction mixture was concentrated to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 43%-73%, 10 min), followed by lyophilization to yield 3-[1-(2-methoxyethyl)imidazol-4-yl]-N-methyl-4-[[4-(trifluoromethyl)phenyl]methylamino]benzenesulfonamide (16.89 mg, 35.36 μmol, 33.0% yield, 98.0% purity) as a white solid. ¹H NMR (400 MHZ, CDCl₃) δ ppm 9.35-9.29 (m, 1H), 7.90 (d, J=1.7 Hz, 1H), 7.62-7.57 (m, 3H), 7.50 (d, J=7.6 Hz, 3H), 7.41 (s, 1H), 6.54 (d, J=8.8 Hz, 1H), 4.61 (d, J=5.9 Hz, 2H), 4.19-4.10 (m, 3H), 3.70 (t, J=5.0 Hz, 2H), 3.39 (s, 3H), 2.63 (d, J=5.6 Hz, 3H); ES-LCMS m/z 469.2 [M+H]⁺.

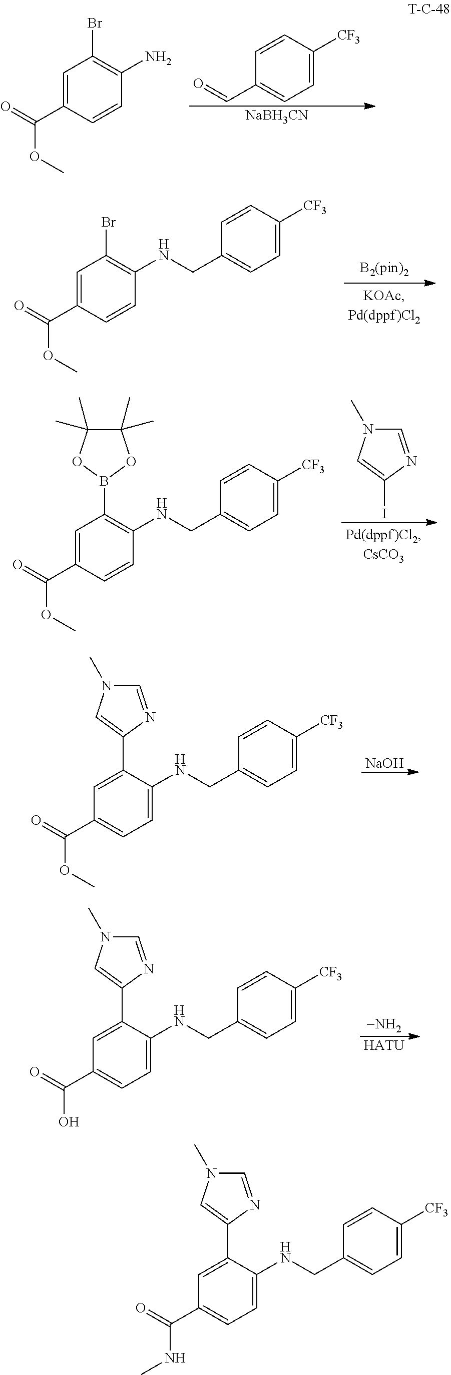

### Step 1: 3-Bromo-4-[[4-(trifluoromethyl)phenyl]methylamino]benzoate

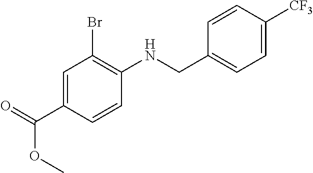 [SMILES: COC(=O)c1ccc(NCc2ccc(C(F)(F)F)cc2)c(Br)c1]

To a solution of 4-(trifluoromethyl)benzaldehyde (3 g, 17.23 mmol, 2.31 mL, 1 eq) in MeOH (50 mL) was added AcOH (2.07 g, 34.46 mmol, 1.97 mL, 2 eq) and methyl 4-amino-3-bromo-benzoate (4.76 g, 20.68 mmol, 1.2 eq). The mixture was stirred at 20° C. for 5 h. NaBH₃CN (2.17 g, 34.46 mmol, 2 eq) was added. The mixture was stirred at 20° C. for 17 h. The reaction mixture was concentrated, diluted with water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 5/1, TLC: PE/EtOAc=5/1, R_{f}=0.54) to yield methyl 3-bromo-4-[[4-(trifluoromethyl)phenyl]methylamino]benzoate (1.1 g, 2.64 mmol, 15.3% yield, 93.0% purity) as a white solid. ¹H NMR (400 MHZ, CDCl₃) δ ppm 8.17 (d, J=2.0 Hz, 1H), 7.81 (dd, J=1.6, 8.6 Hz, 1H), 7.63 (d, J=8.2 Hz, 2H), 7.46 (d, J=7.8 Hz, 2H), 6.50 (d, J=8.6 Hz, 1H), 5.30 (s, 1H), 4.56 (d, J=5.9 Hz, 2H), 3.86 (s, 3H); ES-LCMS m/z 388.1, 390.1 [M+H]⁺.

### Step 2: Methyl 3-(4,4,5,5-tetramethyl-1,3,2-dioxaborolan-2-yl)-4-[[4-(trifluoromethyl)phenyl]methylamino]benzoate

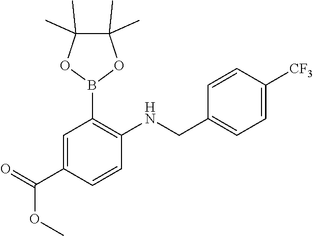 [SMILES: COC(=O)c1ccc(NCc2ccc(C(F)(F)F)cc2)c(B2OC(C)(C)C(C)(C)O2)c1]

To a solution of methyl 3-bromo-4-[[4-(trifluoromethyl)phenyl]methylamino]benzoate (600 mg, 1.44 mmol, 93%, 1 eq), 4,4,5,5-tetramethyl-2-(4,4,5,5-tetramethyl-1,3,2-dioxaborolan-2-yl)-1,3,2-dioxaborolane (730.06 mg, 2.87 mmol, 2 eq) in 1,4-dioxane (10 mL) was added KOAc (282.16 mg, 2.87 mmol, 2 eq) and Pd(PPh₃)₂Cl₂ (100.90 mg, 143.75 μmol, 0.1 eq). The mixture was stirred under N₂ atmosphere at 80° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 5/1, TLC: PE/EtOAc=5/1, R_{f}=0.54) to yield methyl 3-(4,4,5,5-tetramethyl-1,3,2-dioxaborolan-2-yl)-4-[[4-(trifluoromethyl)phenyl]methylamino]benzoate (450 mg, 920.17 μmol, 64.0% yield, 89.0% purity) as a white solid. ¹H NMR (400 MHZ, DMSO-d₆) δ ppm 8.11 (d, J=2.0 Hz, 1H), 7.76 (dd, J=2.0, 8.6 Hz, 1H), 7.72 (d, J=7.8 Hz, 2H), 7.53 (d, J=7.8 Hz, 2H), 6.97 (t, J=5.9 Hz, 1H), 6.52 (d, J=8.6 Hz, 1H), 4.61 (d, J=5.5 Hz, 2H), 3.74 (s, 3H), 1.33 (s, 12H); ES-LCMS m/z 436.3 [M+H]⁺.

### Step 3: Methyl 3-(1-methylimidazol-4-yl)-4-[[4-(trifluoromethyl)phenyl]methylamino]benzoate

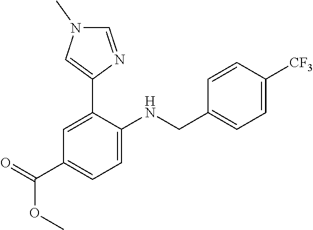 [SMILES: COC(=O)c1ccc(NCc2ccc(C(F)(F)F)cc2)c(-c2cn(C)cn2)c1]

To a solution of methyl 3-(4,4,5,5-tetramethyl-1,3,2-dioxaborolan-2-yl)-4-[[4-(trifluoromethyl)phenyl]methylamino]benzoate (450 mg, 920.17 μmol, 89%, 1.2 eq), 4-iodo-1-methyl-imidazole (159.50 mg, 766.81 μmol, 1 eq) in 1,4-dioxane (10 mL) and H₂O (2 mL) was added Pd(dppf)Cl₂ (56.11 mg, 76.68 μmol, 0.1 eq) and Cs₂CO₃ (249.84 mg, 766.81 μmol, 1 eq). The mixture was stirred under N₂ atmosphere at 80° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.38) to yield methyl 3-(1-methylimidazol-4-yl)-4-[[4-(trifluoromethyl)phenyl]methylamino]benzoate (200 mg, 410.92 μmol, 53.5% yield, 80.0% purity) as yellow oil. ¹H NMR (400 MHZ, CDCl₃) δ ppm 9.18 (s, 1H), 8.12 (d, J=2.2 Hz, 1H), 7.73 (dd, J=2.0, 8.6 Hz, 1H), 7.60-7.55 (m, 2H), 7.49 (d, J=10.8 Hz, 3H), 7.32 (d, J=1.2 Hz, 1H), 6.50 (d, J=8.8 Hz, 1H), 4.61 (d, J=5.1 Hz, 2H), 3.86 (s, 3H), 3.78 (s, 3H); ES-LCMS m z 390.2 [M+H]⁺.

### Step 4: 3-(1-Methylimidazol-4-yl)-4-[[4-(trifluoromethyl)phenyl]methylamino]benzoic acid

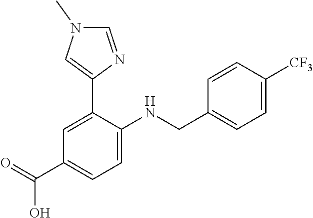 [SMILES: Cn1cnc(-c2cc(C(=O)O)ccc2NCc2ccc(C(F)(F)F)cc2)c1]

To a solution of methyl 3-(1-methylimidazol-4-yl)-4-[[4-(trifluoromethyl)phenyl]methylamino]benzoate (200 mg, 410.92 μmol, 80%, 1 eq) in THF (2 mL) and MeOH (2 mL) was added NaOH (109.58 mg, 410.92 μmol, 2 mL, 15%, 1 eq). The mixture was stirred at 20° C. for 12 h. The reaction mixture was concentrated to yield a residue which was dissolved HCl (1M, 10 mL), adjusted pH to 5˜6. The reaction mixture was quenched by addition of water (20 mL) and extracted with EtOAc (20 mL×3). The combined organic layers were washed with brine (10 mL), dried over Na₂SO₄, filtered and concentrated under reduced pressure to yield 3-(1-methylimidazol-4-yl)-4-[[4-(trifluoromethyl)phenyl]methylamino]benzoic acid (100 mg, 242.44 μmol, 59.0% yield, 91.0% purity) as a white solid, which was used in the next step without further purification. ¹H NMR (400 MHZ, DMSO-d₆) δ ppm 12.13 (s, 1H), 9.41-9.31 (m, 1H), 8.02 (d, J=2.0 Hz, 1H), 7.78 (s, 1H), 7.70 (d, J=9.3 Hz, 3H), 7.55 (d, J=8.6 Hz, 3H), 6.55 (d, J=8.6 Hz, 1H), 4.65 (d, J=5.4 Hz, 2H), 3.73 (s, 3H); ES-LCMS m/z 376.2 [M+H]⁺.

### Step 5: N-Methyl-3-(1-methylimidazol-4-yl)-4-[[4-(trifluoromethyl)phenyl]methylamino]benzamide

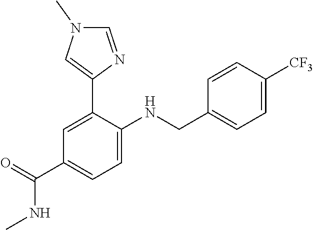 [SMILES: CNC(=O)c1ccc(NCc2ccc(C(F)(F)F)cc2)c(-c2cn(C)cn2)c1]

To a solution of 3-(1-methylimidazol-4-yl)-4-[[4-(trifluoromethyl)phenyl]methylamino]benzoic acid (70 mg, 169.71 μmol, 91%, 1 eq) in THF (3 mL) was added Et₃N (51.52 mg, 509.13 μmol, 70.86 μL, 3 eq) and methanamine; hydrochloride (57.29 mg, 848.56 μmol, 5 eq) and HATU (129.06 mg, 339.42 μmol, 2 eq). The mixture was stirred at 20° C. for 1 h. The reaction mixture was concentrated to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 36%-66%, 10 min), followed by lyophilization to yield N-methyl-3-(1-methylimidazol-4-yl)-4-[[4-(trifluoromethyl)phenyl]methylamino]benzamide (27.73 mg, 71.40 μmol, 42.0% yield, 100.0% purity) as a white solid. ¹H NMR (400 MHZ, DMSO-d₆) δ ppm 9.13 (t, J=6.5 Hz, 1H), 8.05 (d, J=4.3 Hz, 1H), 7.98 (d, J=2.0 Hz, 1H), 7.77 (s, 1H), 7.69 (d, J=7.8 Hz, 2H), 7.65 (s, 1H), 7.55 (d, J=8.2 Hz, 2H), 7.46 (d, J=8.6 Hz, 1H), 6.52 (d, J=8.6 Hz, 1H), 4.62 (d, J=5.9 Hz, 2H), 3.75 (s, 3H), 2.74 (d, J=4.3 Hz, 3H); ES-LCMS m/z 389.2 [M+H]⁺.

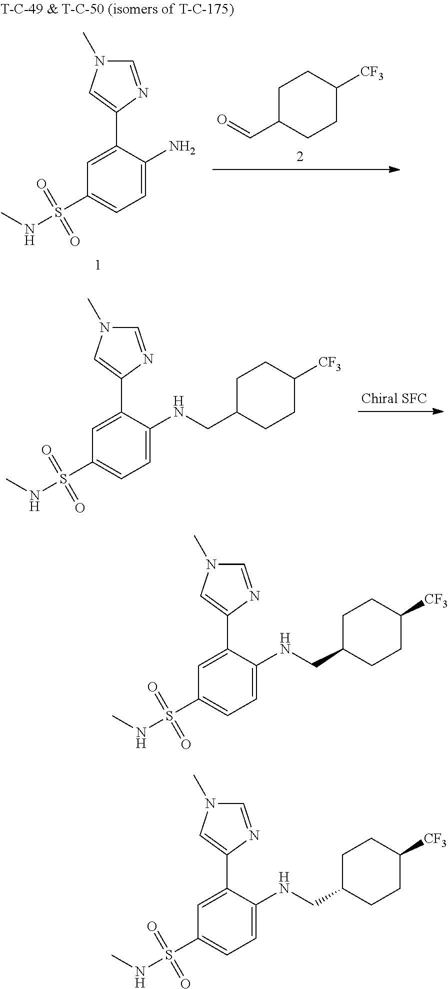

### Step 1: N-Methyl-3-(1-methylimidazol-4-yl)-4-[[4-(trifluoromethyl)cyclohexyl]methylamino]benzenesulfonamide and N-methyl-3-(1-methylimidazol-4-yl)-4-[[4-(trifluoromethyl)cyclohexyl]methylamino]benzenesulfonamide

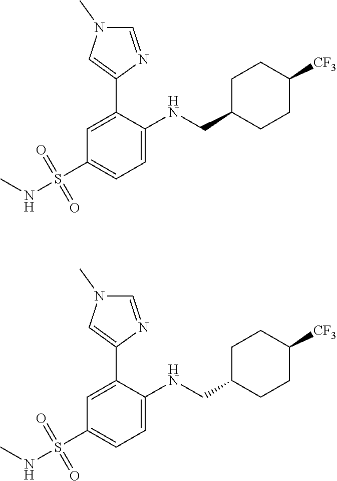 [SMILES: CNS(=O)(=O)c1ccc(NC[C@H]2CC[C@@H](C(F)(F)F)CC2)c(-c2cn(C)cn2)c1 ; CNS(=O)(=O)c1ccc(NC[C@H]2CC[C@H](C(F)(F)F)CC2)c(-c2cn(C)cn2)c1]

To a stirred solution of 4-amino-N-methyl-3-(1-methylimidazol-4-yl)benzenesulfonamide (155.60 mg, 555.04 μmol, 95.0% purity, 1 eq) and 4-(trifluoromethyl)cyclohexanecarbaldehyde (200 mg, 777.06 μmol, 70.0% purity, 1.4 eq) in MeOH (10 mL) was added AcOH (6.67 mg, 111.01 μmol, 6.35 μL, 0.2 eq). The reaction mixture was stirred under N₂ atmosphere at 25° C. for 1 h. NaBH₃CN (174.39 mg, 2.78 mmol, 5 eq) was added and the reaction mixture was stirred under N₂ atmosphere at 25° C. for 11 h. The reaction mixture was concentrated under reduced pressure, diluted with H₂O (15 mL), adjust pH to 8 by sat aq NaHCO₃ and extracted with EtOAc (30 mL×3). The combined organic layers were washed with brine (20 mL), dried over Na₂SO₄, filtered and concentrated under reduced pressure to yield a residue which was purified by preparative HPLC (column: Phenomenex Synergi C18 150*30 mm*4 μm; mobile phase: [water (0.05% HCl)-ACN]; B %: 30%-50%, 10 min) to yield crude product which was separated by chiral SFC (column: Phenomenex-Cellulose-2 (250 mm*30 mm, 5 μm); mobile phase: [0.1% NH₃H₂O ETOH]; B %: 40%-40%, min) to yield Peak 1 and Peak 2. Peak 1 was concentrated under reduced pressure to yield a residue which was dissolved in MeCN (20 mL) and H₂O (40 mL) and lyophilized to yield N-methyl-3-(1-methylimidazol-4-yl)-4-[[4-(trifluoromethyl)cyclohexyl]methylamino]benzenesulfonamide (15.11 mg, 35.10 μmol, 6.3% yield, 100.0% purity, SFC: Rₜ=4.596, ee=100.0%) as a gray solid. ¹H NMR (500 MHz, DMSO-d₆) δ ppm 8.80 (t, J=5.2 Hz, 1H), 7.78 (s, 1H), 7.76 (d, J=2.3 Hz, 1H), 7.64 (d, J=1.1 Hz, 1H), 7.40 (dd, J=2.1, 8.7 Hz, 1H), 6.98 (d, J=5.2 Hz, 1H), 6.76 (d, J=8.7 Hz, 1H), 3.73 (s, 3H), 3.25-3.17 (m, 2H), 2.36 (d, J=5.0 Hz, 4H), 1.96 (s, 1H), 1.67-1.65 (m, 4H), 1.62-1.52 (m, 4H); ES-LCMS m/z 431.3 [M+H]⁺. Peak 2 was concentrated under reduced pressure to yield a residue which was dissolved in MeCN (20 mL) and H₂O (40 mL) and lyophilized to yield N-methyl-3-(1-methylimidazol-4-yl)-4-[[4-(trifluoromethyl)cyclohexyl]methylamino]benzenesulfonamide (45.85 mg, 104.85 μmol, 18.9% yield, 98.4% purity, SFC: Rₜ=4.950, ee=98.2%) as a gray solid. ¹H NMR (500 MHz, DMSO-d₆) δ ppm 8.88 (t, J=5.5 Hz, 1H), 7.79 (s, 1H), 7.75 (d, J=2.1 Hz, 1H), 7.64 (d, J=0.9 Hz, 1H), 7.40 (dd, J=2.1, 8.7 Hz, 1H), 6.97 (q, J=5.0 Hz, 1H), 6.73 (d, J=8.9 Hz, 1H), 3.73 (s, 3H), 3.10 (t, J=6.0 Hz, 2H), 2.36 (d, J=5.0 Hz, 3H), 2.27-2.15 (m, 1H), 1.93-1.89 (m, 4H), 1.66-1.58 (m, 1H), 1.31-1.22 (m, 2H), 1.15-1.06 (m, 2H); ES-LCMS m/z 431.1 [M+H]⁺.

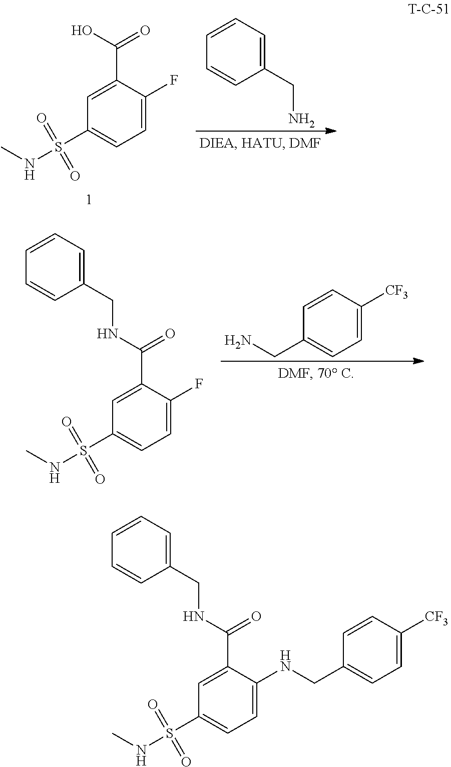

### Step 1: N-Benzyl-2-fluoro-5-(methylsulfamoyl)benzamide

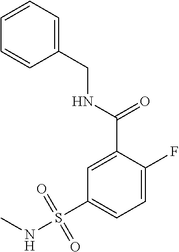 [SMILES: CNS(=O)(=O)c1ccc(F)c(C(=O)NCc2ccccc2)c1]

To a solution of phenylmethanamine (206.76 mg, 1.93 mmol, 210.33 μL, 1 eq) in DMF (3 mL) was added DIEA (748.12 mg, 5.79 mmol, 1.01 mL, 3 eq), 2-fluoro-5-(methylsulfamoyl)benzoic acid (500 mg, 1.93 mmol, 90% purity, 1 eq) and HATU (1.32 g, 3.47 mmol, 1.8 eq). The mixture was stirred at 25° C. for 0.5 h. The mixture was added water (15 mL) and extracted with EtOAc (20 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 1/1, TLC: PE/EtOAc=0/1, R_{f}=0.7) to yield N-benzyl-2-fluoro-5-(methylsulfamoyl)benzamide (300 mg, 856.20 μmol, 44.4% yield, 92.0% purity) as a white solid. ¹H NMR (400 MHZ, CDCl₃) δ ppm 8.61 (dd, J=2.7, 7.0 Hz, 1H), 8.00 (ddd, J=2.7, 4.7, 8.6 Hz, 1H), 7.38-7.34 (m, 4H), 7.33-7.30 (m, 1H), 7.29-7.26 (m, 1H), 7.00 (s, 1H), 4.72-4.68 (m, 3H), 2.67 (d, J=5.5 Hz, 3H); ES-LCMS m/z 323.2 [M+H]⁺.

### Step 2: N-Benzyl-5-(methylsulfamoyl)-2-[[4-(trifluoromethyl)phenyl]methylamino]benzamide

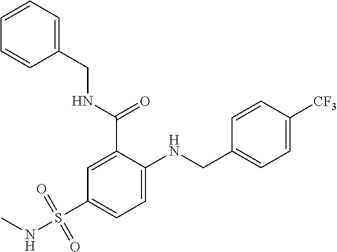 [SMILES: CNS(=O)(=O)c1ccc(NCc2ccc(C(F)(F)F)cc2)c(C(=O)NCc2ccccc2)c1]

To a solution of N-benzyl-2-fluoro-5-(methylsulfamoyl)benzamide (150 mg, 428.10 μmol, 92.0% purity, 1 eq) in DMF (3 mL) was added [4-(trifluoromethyl)phenyl]methanamine (149.96 mg, 856.20 μmol, 121.92 μL, 2 eq). The mixture was stirred at 70° C. for 4 h. The reaction mixture was concentrated to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 53%-83%, 10 min), followed by lyophilization to yield N-benzyl-5-(methylsulfamoyl)-2-[[4-(trifluoromethyl)phenyl]methylamino]benzamide (76.67 mg, 160.57 μmol, 37.5% yield, 100% purity) as a white solid. ¹H NMR (500 MHZ, CDCl₃) δ ppm 8.95 (t, J=5.8 Hz, 1H), 7.90 (d, J=2.1 Hz, 1H), 7.63 (dd, J=2.1, 8.9 Hz, 1H), 7.61 (s, 1H), 7.60 (s, 1H), 7.46 (d, J=8.1 Hz, 2H), 7.41-7.34 (m, 4H), 7.34-7.29 (m, 1H), 6.69 (s, 1H), 6.59 (d, J=9.0 Hz, 1H), 4.60 (d, J=5.6 Hz, 2H), 4.54 (d, J=6.0 Hz, 2H), 4.24 (q, J=5.4 Hz, 1H), 2.59 (d, J=5.5 Hz, 3H); ES-LCMS m/z 478.2 [M+H]⁺.

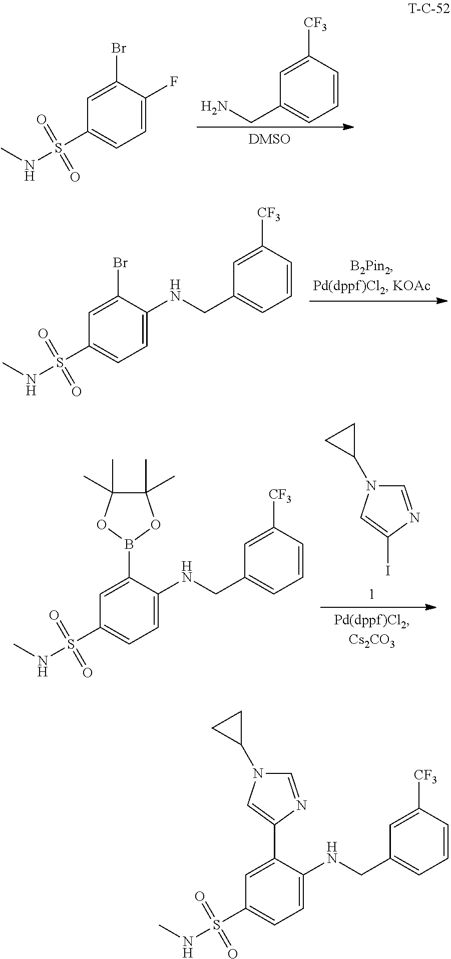 [SMILES: CNS(=O)(=O)c1ccc(F)c(Br)c1 ; NCc1cccc(C(F)(F)F)c1 ; CNS(=O)(=O)c1ccc(NCc2cccc(C(F)(F)F)c2)c(Br)c1 ; CNS(=O)(=O)c1ccc(NCc2cccc(C(F)(F)F)c2)c(B2OC(C)(C)C(C)(C)O2)c1 ; Ic1cn(C2CC2)cn1 ; CNS(=O)(=O)c1ccc(NCc2cccc(C(F)(F)F)c2)c(-c2cn(C3CC3)cn2)c1]

### Step 1: 3-Bromo-N-methyl-4-[[3-(trifluoromethyl)phenyl]methylamino]benzenesulfonamide

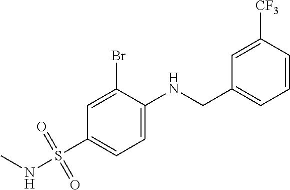 [SMILES: CNS(=O)(=O)c1ccc(NCc2cccc(C(F)(F)F)c2)c(Br)c1]

To a solution of 3-bromo-4-fluoro-N-methyl-benzenesulfonamide (400 mg, 1.49 mmol, 100% purity, 1 eq) in DMSO (6 mL) was added [3-(trifluoromethyl)phenyl]methanamine (522.64 mg, 2.98 mmol, 428.39 μL, 2 eq). The mixture was stirred at 140° C. for 12 h. The mixture was diluted with water (20 mL) and extracted with EtOAc (20 mL×3). The combined organic layers were dried over Na₂SO₄, filtered and concentrated under reduced pressure to yield a residue which was purified by flash silica gel chromatography (from PE/EtOAc=100/1 to 3/1, TLC: PE/EtOAc=3/1, R_{f}=0.30) to yield 3-bromo-N-methyl-4-[[3-(trifluoromethyl)phenyl]methylamino]benzenesulfonamide (590 mg, 1.32 mmol, 88.8% yield, 95.0% purity) as a brown oil. ¹H NMR (400 MHZ, CDCl₃) δ ppm 7.97 (d, J=2.0 Hz, 1H), 7.65-7.56 (m, 3H), 7.56-7.48 (m, 2H), 6.57 (d, J=8.6 Hz, 1H), 5.40-5.25 (m, 1H), 4.56 (d, J=5.9 Hz, 2H), 4.25 (q, J=5.7 Hz, 1H), 2.65 (d, J=5.5 Hz, 3H); ES-LCMS m/z 425.1 [M+H]⁺.

### Step 2: N-Methyl-3-(4,4,5,5-tetramethyl-1,3,2-dioxaborolan-2-yl)-4-[[3-(trifluoromethyl)phenyl]methylamino]benzenesulfonamide

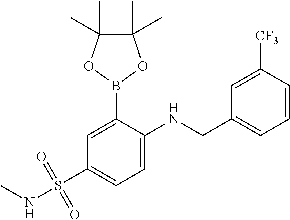 [SMILES: CNS(=O)(=O)c1ccc(NCc2cccc(C(F)(F)F)c2)c(B2OC(C)(C)C(C)(C)O2)c1]

A mixture of 3-bromo-N-methyl-4-[[3-(trifluoromethyl)phenyl]methylamino]benzenesulfonamide (200 mg, 448.91 μmol, 95% purity, 1 eq), 4,4,5,5-tetramethyl-2-(4,4,5,5-tetramethyl-1,3,2-dioxaborolan-2-yl)-1,3,2-dioxaborolane (136.80 mg, 538.72 μmol, 1.2 eq), KOAc (133.00 mg, 1.36 mmol, 3.02 eq) and Pd(PPh₃)₂Cl₂ (31.51 mg, 44.89 μmol, 0.1 eq) in 1,4-dioxane (4 mL) was degassed and purged with N₂ for 3 times and the mixture was stirred at 110° C. under N₂ atmosphere for 12 h. The mixture was diluted with water (20 mL) and extracted with EtOAc (25 mL×3). The combined organic layers were dried over Na₂SO₄, filtered and concentrated under reduced pressure to yield a residue which was purified by flash silica gel chromatography (from PE/EtOAc=100/1 to 4/1, TLC: PE/EtOAc=3/1, R_{f}=0.70) to yield N-methyl-3-(4,4,5,5-tetramethyl-1,3,2-dioxaborolan-2-yl)-4-[[3-(trifluoromethyl)phenyl]methylamino]benzenesulfonamide (100 mg, 138.21 μmol, 30.8% yield, 65.0% purity) as colorless oil. ¹H NMR (400 MHZ, CDCl₃) δ ppm 8.14 (d, J=2.4 Hz, 1H), 7.70 (dd, J=2.2, 8.8 Hz, 1H), 7.63 (s, 1H), 7.59-7.46 (m, 4H), 6.92-6.85 (m, 1H), 6.49 (d, J=8.8 Hz, 1H), 4.51 (d, J=5.6 Hz, 2H), 2.62 (d, J=5.6 Hz, 3H), 1.25 (s, 12H); ES-LCMS m/z 471.2 [M+H]⁺.

### Step 3: 3-(1-Cyclopropylimidazol-4-yl)-N-methyl-4-[[3-(trifluoromethyl)phenyl]methylamino]benzenesulfonamide

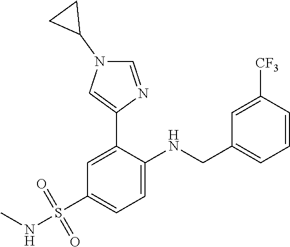 [SMILES: CNS(=O)(=O)c1ccc(NCc2cccc(C(F)(F)F)c2)c(-c2cn(C3CC3)cn2)c1]

A mixture of □-methyl-3-(4,4,5,5-tetramethyl-1,3,2-dioxaborolan-2-yl)-4-[[3-(trifluoromethyl)phenyl]methylamino]benzenesulfonamide (100 mg, 138.21 μmol, 65% purity, 1 eq), 1-cyclopropyl-4-iodo-imidazole (80.86 mg, 138.21 μmol, 40% purity, 1 eq), Cs₂CO₃ (135.09 mg, 414.62 μmol, 3 eq) and Pd(dppf)Cl₂ (10.11 mg, 13.82 μmol, 0.1 eq) in 1,4-dioxane (1.5 mL) and H₂O (0.5 mL) was degassed and purged with N₂ for 3 times and the mixture was stirred under N₂ atmosphere at 100° C. for 12 h. The mixture was diluted with water (10 mL) and extracted with EtOAc (10 mL×3). The combined organic layers were dried over Na₂SO₄, filtered and concentrated under reduced pressure to yield a residue which was purified by preparative HPLC (column: Welch Xtimate C18 150*25 mm*5 μm; mobile phase: [water (10 mM NH₄HCO₃)-ACN]; B %: 45%-75%, 10 min), followed by lyophilization to yield 3-(1-cyclopropylimidazol-4-yl)-N-methyl-4-[[3-(trifluoromethyl)phenyl]methylamino]benzenesulfonamide (11.42 mg, 25.35 μmol, 18.3% yield, 100.0% purity) as a gray solid. ¹H NMR (400 MHZ, CDCl₃) δ ppm 9.23 (t, J=6.0 Hz, 1H), 7.87 (d, J=2.3 Hz, 1H), 7.64 (s, 1H), 7.60 (d, J=1.1 Hz, 1H), 7.57-7.44 (m, 4H), 7.40 (d, J=1.2 Hz, 1H), 6.56 (d, J=8.9 Hz, 1H), 4.59 (d, J=5.6 Hz, 2H), 4.13 (q, J=5.3 Hz, 1H), 3.48-3.39 (m, 1H), 2.63 (d, J=5.6 Hz, 3H), 1.13-1.01 (m, 4H); ES-LCMS m/z 451.2 [M+H]⁺.

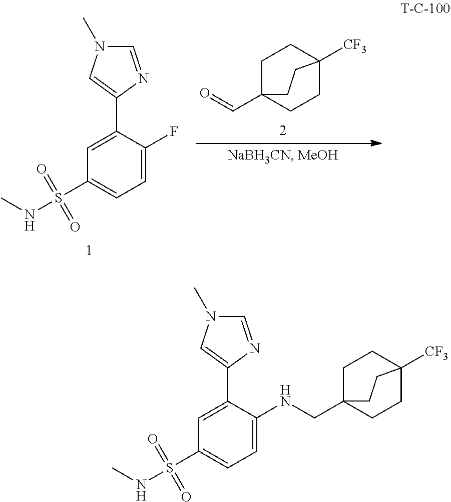 [SMILES: CNS(=O)(=O)c1ccc(F)c(-c2cn(C)cn2)c1 ; O=CC12CCC(C(F)(F)F)(CC1)CC2 ; CNS(=O)(=O)c1ccc(NCC23CCC(C(F)(F)F)(CC2)CC3)c(-c2cn(C)cn2)c1]

### Step 1: N-Methyl-3-(1-methylimidazol-4-yl)-4-[[4-(trifluoromethyl)-1-bicyclo[2.2.2]octanyl]methylamino]benzenesulfonamide

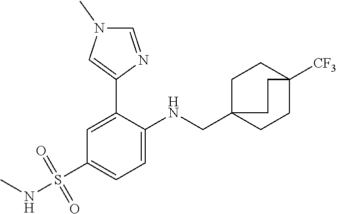 [SMILES: CNS(=O)(=O)c1ccc(NCC23CCC(C(F)(F)F)(CC2)CC3)c(-c2cn(C)cn2)c1]

To a solution of 4-amino-N-methyl-3-(1-methylimidazol-4-yl)benzenesulfonamide (55 mg, 196.19 μmol, 95% purity, 1 eq) in MeOH (5 mL) was added 4-(trifluoromethyl)bicyclo[2.2.2]octane-1-carbaldehyde (67.43 mg, 196.19 μmol, 60% purity, 1 eq), followed by 1 drop of AcOH. The mixture was stirred at 25° C. for 2 h. NaBH₃CN (36.99 mg, 588.58 μmol, 3 eq) was added and the mixture was stirred at 25° C. for 16 h. The solvent was removed to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 50%-80%, 10 min), followed by lyophilization to yield N-methyl-3-(1-methylimidazol-4-yl)-4-[[4-(trifluoromethyl)-1-bicyclo[2.2.2]octanyl]methylamino]benzenesulfonamide (8.74 mg, 19.14 μmol, 9.8% yield, 100.0% purity) as a white solid. ¹H NMR (400 MHZ, CDCl₃) δ ppm 8.75 (br s, 1H), 7.81 (d, J=2.2 Hz, 1H), 7.53 (dd, J=2.2, 8.8 Hz, 1H), 7.49 (s, 1H), 7.26 (d, J=1.2 Hz, 1H), 6.64 (d, J=8.8 Hz, 1H), 4.11 (q, J=5.4 Hz, 1H), 3.76 (s, 3H), 3.00 (d, J=5.4 Hz, 2H), 2.61 (d, J=5.4 Hz, 3H), 1.77-1.67 (m, 6H), 1.64-1.56 (m, 6H); ES-LCMS m/z 457.3 [M+H]⁺.

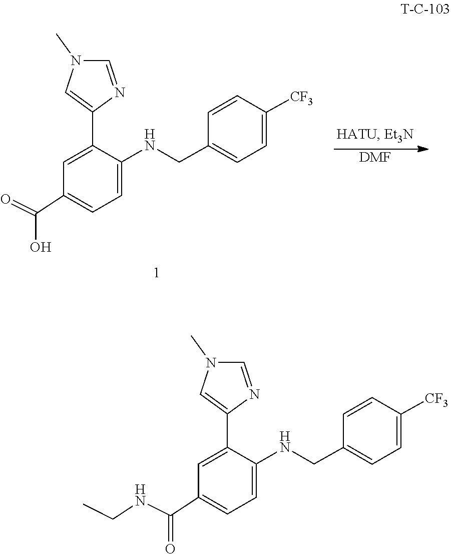

### Step 1: N-Ethyl-3-(1-methylimidazol-4-yl)-4-[[4-(trifluoromethyl)phenyl]methylamino]benzamide

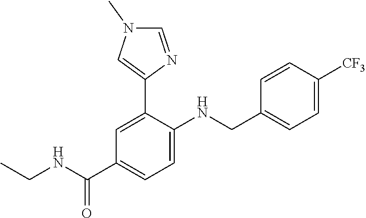 [SMILES: CCNC(=O)c1ccc(NCc2ccc(C(F)(F)F)cc2)c(-c2cn(C)cn2)c1]

To a solution of ethanamine (102.70 mg, 2.28 mmol, 149.05 μL, 9 eq) in DMF (5 mL) was added 3-(1-methylimidazol-4-yl)-4-[[4-(trifluoromethyl)phenyl]methylamino]benzoic acid (100 mg, 253.10 μmol, 95% purity, 1 eq), Et₃N (76.83 mg, 759.30 μmol, 105.69 μL, 3 eq) and HATU (173.23 mg, 455.58 μmol, 1.8 eq). The mixture was stirred at 20° C. for 1 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 40%-70%, 10 min) and lyophilized to yield N-ethyl-3-(1-methylimidazol-4-yl)-4-[[4-(trifluoromethyl)phenyl]methylamino]benzamide (40 mg, 98.77 μmol, 39.0% yield, 99.0% purity) as a white solid. ¹H NMR (400 MHZ, CDCl₃) δ ppm 8.95 (s, 1H), 7.98 (d, J=2.2 Hz, 1H), 7.59-7.55 (m, 2H), 7.49 (d, J=9.5 Hz, 3H), 7.36 (dd, J=2.2, 8.6 Hz, 1H), 7.32 (d, J=1.2 Hz, 1H), 6.47 (d, J=8.6 Hz, 1H), 5.94 (s, 1H), 4.60 (d, J=5.4 Hz, 2H), 3.77 (s, 3H), 3.51-3.44 (m, 2H), 1.23 (t, J=7.2 Hz, 3H); ES-LCMS m/z 403.3 [M+H]⁺.

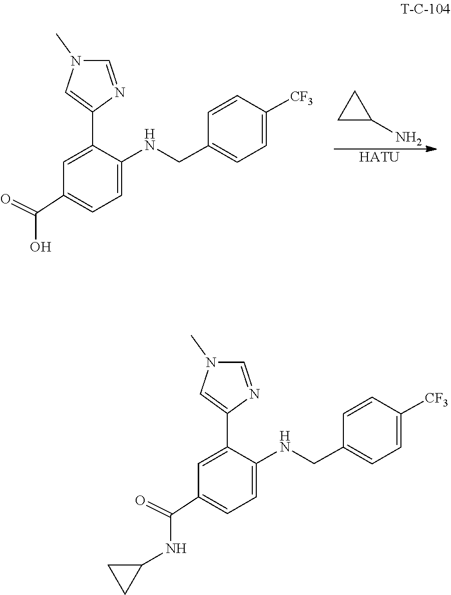

### Step 1: N-Cyclopropyl-3-(1-methylimidazol-4-yl)-4-[[4-(trifluoromethyl)phenyl]methylamino]benzamide

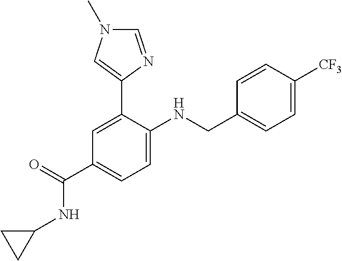 [SMILES: Cn1cnc(-c2cc(C(=O)NC3CC3)ccc2NCc2ccc(C(F)(F)F)cc2)c1]

To a solution of cyclopropanamine (57.80 mg, 1.01 mmol, 70.15 μL, 4 eq) in THF (3 mL) was added DIEA (65.42 mg, 506.20 μmol, 88.17 μL, 2 eq) and 3-(1-methylimidazol-4-yl)-4-[[4-(trifluoromethyl)phenyl]methylamino]benzoic acid (100 mg, 253.10 μmol, 95%, 1 eq) and HATU (192.47 mg, 506.20 μmol, 2 eq). The mixture was stirred at 20° C. for 1 h. The reaction mixture was concentrated to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 40%-70%, 10 min), followed by lyophilization to yield N-cyclopropyl-3-(1-methylimidazol-4-yl)-4-[[4-(trifluoromethyl)phenyl]methylamino]benzamide (73.29 mg, 176.85 μmol, 69.8% yield, 100.0% purity) as a white solid. ¹H NMR (400 MHZ, CDCl₃) δ ppm 8.98 (s, 1H), 7.96 (d, J=2.0 Hz, 1H), 7.60-7.54 (m, 2H), 7.48 (d, J=8.2 Hz, 3H), 7.34-7.29 (m, 2H), 6.45 (d, J=8.6 Hz, 1H), 6.07 (s, 1H), 4.59 (d, J=5.5 Hz, 2H), 3.78 (s, 3H), 2.87 (m, 1H), 0.87-0.81 (m, 2H), 0.61-0.54 (m, 2H); ES-LCMS m/z 415.2 [M+H]⁺.

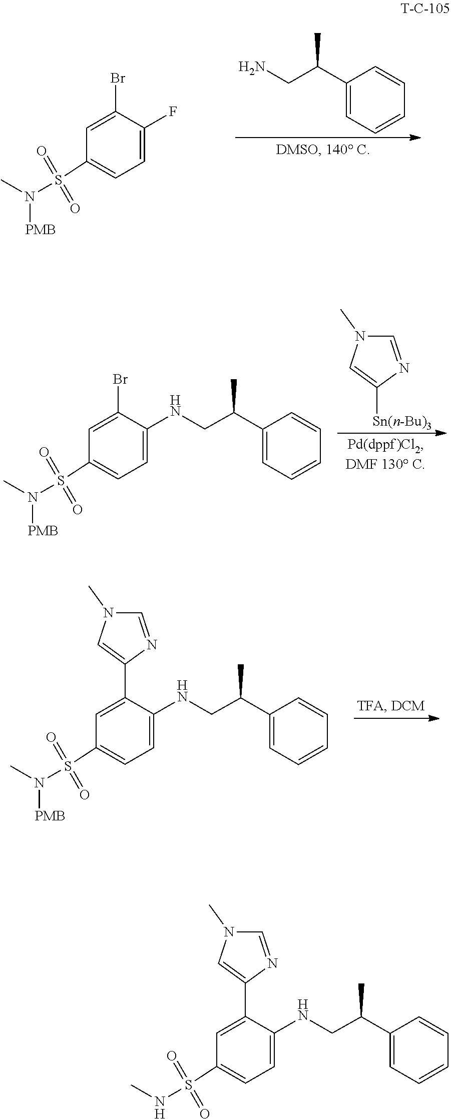

### Step 1: 3-Bromo-N-[(4-methoxyphenyl)methyl]-N-methyl-4-[[(2S)-2-phenylpropyl]amino]benzenesulfonamide

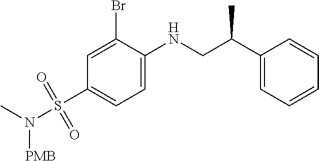

To a solution of 3-bromo-4-fluoro-N-[(4-methoxyphenyl)methyl]-N-methyl-benzenesulfonamide (300 mg, 734.06 μmol, 95% purity, 1 eq) in DMSO (5 mL) was added (2S)-2-phenylpropan-1-amine (198.50 mg, 1.47 mmol, 210.05 μL, 2 eq). The mixture was stirred at 140° C. for 2 h. The mixture was added water (20 mL) and extracted with EtOAc (20 mL×3). The combined organic layers were washed with brine (10 mL), dried over Na₂SO₄, filtered and concentrated under reduced pressure to give a residue which was purified by flash silica gel chromatography (from PE/EtOAc=1/0 to 3/1, TLC: PE/EtOAc=5/1, R_{f}=0.55) to yield 3-bromo-N-[(4-methoxyphenyl)methyl]-N-methyl-4-[[(2S)-2-phenylpropyl]amino]benzenesulfonamide (336 mg, 634.02 μmol, 86.3% yield, 95.0% purity) as a white solid. ¹H NMR (400 MHZ, CDCl₃) 0 ppm 7.83 (d, J=2.0 Hz, 1H), 7.61 (dd, J=2.0, 8.6 Hz, 1H), 7.39-7.34 (m, 2H), 7.30-7.26 (m, 2H), 7.25 (s, 1H), 7.21 (d, J=8.6 Hz, 2H), 6.86 (d, J=8.6 Hz, 2H), 6.65 (d, J=8.6 Hz, 1H), 4.79 (t, J=5.3 Hz, 1H), 4.03 (s, 2H), 3.80 (s, 3H), 3.47-3.41 (m, 1H), 3.36-3.29 (m, 1H), 3.15-3.07 (m, 1H), 2.53 (s, 3H), 1.41 (d, J=7.0 Hz, 3H); ES-LCMS m/z 505.1 [M+H]⁺.

### Step 2: N-[(4-Methoxyphenyl)methyl]-N-methyl-3-(1-methylimidazol-4-yl)-4-[[(2S)-2-phenylpropyl]amino]benzenesulfonamide

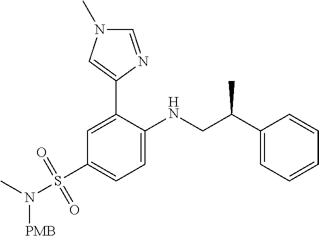

To a solution of 3-bromo-N-[(4-methoxyphenyl)methyl]-N-methyl-4-[[(2S)-2-phenylpropyl]amino]benzenesulfonamide (330 mg, 622.70 μmol, 95% purity, 1 eq) in DMF (4 mL) was added tributyl-(1-methylimidazol-4-yl)stannane (486.56 mg, 1.25 mmol, 95% purity, 2 eq) and Pd(dppf)Cl₂ (45.56 mg, 62.27 μmol, 0.1 eq). The mixture was stirred under N₂ atmosphere at 130° C. for 3 h. The mixture was diluted with water (20 mL) and extracted with EtOAc (20 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 1/1, TLC: PE/EtOAc=3/1, R_{f}=0.2) to yield N-[(4-methoxyphenyl)methyl]-N-methyl-3-(1-methylimidazol-4-yl)-4-[[(2S)-2-phenylpropyl]amino]benzenesulfonamide (310 mg, 583.58 μmol, 93.7% yield, 95.0% purity) as blue oil. ¹H NMR (400 MHZ, CDCl₃) δ ppm 8.61 (s, 1H), 7.75 (d, J=2.3 Hz, 1H), 7.53 (dd, J=2.2, 8.8 Hz, 1H), 7.39 (s, 1H), 7.36-7.26 (m, 5H), 7.26-7.15 (m, 5H), 6.84 (d, J=8.6 Hz, 2H), 6.70 (d, J=9.0 Hz, 1H), 4.01 (s, 2H), 3.79 (s, 3H), 3.73 (s, 3H), 3.47-3.36 (m, 2H), 3.21-3.12 (m, 1H), 2.51 (s, 3H), 1.43 (d, J=7.0 Hz, 3H); ES-LCMS m/z 505.3 [M+H]⁺.

### Step 3: N-Methyl-3-(1-methylimidazol-4-yl)-4-[[(2S)-2-phenylpropyl]amino]benzenesulfonamide

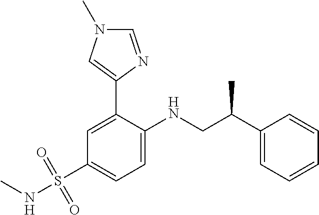 [SMILES: CNS(=O)(=O)c1ccc(NC[C@@H](C)c2ccccc2)c(-c2cn(C)cn2)c1]

To a solution of N-[(4-methoxyphenyl)methyl]-N-methyl-3-(1-methylimidazol-4-yl)-4-[[(2S)-2-phenylpropyl]amino]benzenesulfonamide (150 mg, 297.24 μmol, 1 eq) in DCM (3 mL) was added TFA (1.54 g, 13.51 mmol, 1 mL, 45.44 eq). The mixture was stirred at 25° C. for 0.5 h. The reaction mixture was concentrated to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 40%-70%, 10 min), followed by lyophilization to yield N-methyl-3-(1-methylimidazol-4-yl)-4-[[(2S)-2-phenylpropyl]amino]benzenesulfonamide (48.27 mg, 125.54 μmol, 42.2% yield, 100.0% purity) as a white solid. ¹H NMR (400 MHZ, CDCl₃) δ ppm 8.60 (s, 1H), 7.80 (d, J=2.3 Hz, 1H), 7.54 (dd, J=2.2, 8.8 Hz, 1H), 7.37 (s, 1H), 7.34-7.31 (m, 1H), 7.31-7.27 (m, 3H), 7.26-7.20 (m, 1H), 7.18 (d, J=0.8 Hz, 1H), 6.67 (d, J=8.6 Hz, 1H), 4.20-4.10 (m, 1H), 3.72 (s, 3H), 3.45-3.35 (m, 2H), 3.20-3.09 (m, 1H), 2.60 (d, J=5.5 Hz, 3H), 1.41 (d, J=7.0 Hz, 3H); ES-LCMS m/z 385.3 [M+H]⁺.

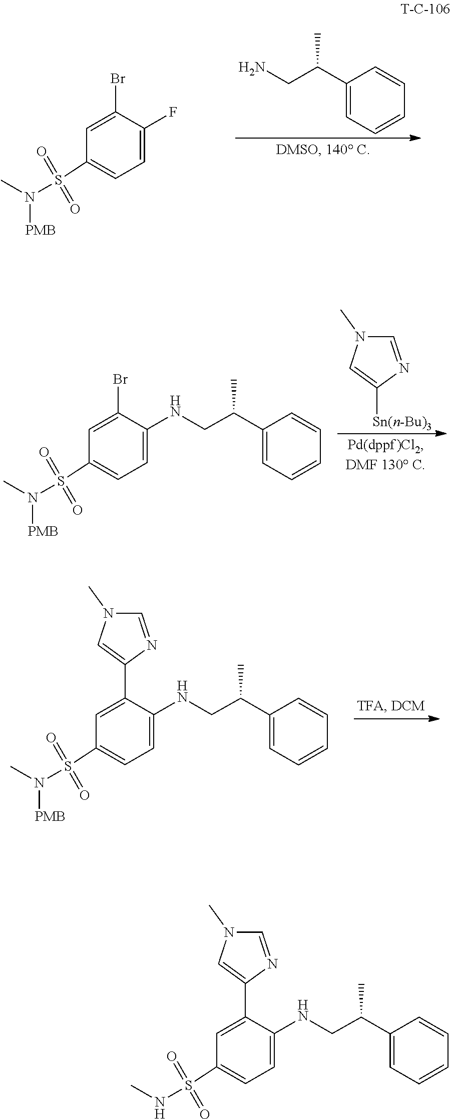

### Step 1: 3-Bromo-N-[(4-methoxyphenyl)methyl]-N-methyl-4-[[(2R)-2-phenylpropyl]amino]benzenesulfonamide

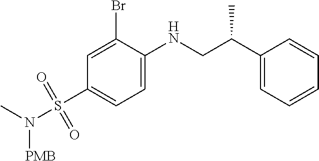

To a solution of 3-bromo-4-fluoro-N-[(4-methoxyphenyl)methyl]-N-methyl-benzenesulfonamide (100 mg, 244.69 μmol, 95% purity, 1 eq) in DMSO (2 mL) was added (2R)-2-phenylpropan-1-amine (66.17 mg, 489.37 μmol, 70.02 μL, 2 eq). The mixture was stirred at 140° C. for 2 h. The mixture was added water (20 mL) and extracted with EtOAc (20 mL×3). The combined organic layers were washed with brine (10 mL), dried over Na₂SO₄, filtered and concentrated under reduced pressure to yield 3-bromo-N-[(4-methoxyphenyl)methyl]-N-methyl-4-[[(2R)-2-phenylpropyl]amino]benzenesulfonamide (120 mg, 166.85 μmol, 68.1% yield, 70.0% purity) as a yellow oil. ¹H NMR (500 MHZ, CDCl₃) δ ppm 7.82 (d, J=2.1 Hz, 1H), 7.60 (dd, J=2.1, 8.6 Hz, 1H), 7.36 (d, J=7.6 Hz, 2H), 7.28 (d, J=7.3 Hz, 1H), 7.25 (s, 1H), 7.21 (d, J=8.7 Hz, 3H), 6.86 (d, J=8.5 Hz, 2H), 6.65 (d, J=8.7 Hz, 1H), 4.79 (t, J=5.3 Hz, 1H), 4.03 (s, 2H), 3.80 (s, 3H), 3.44 (td, J=6.4, 12.5 Hz, 1H), 3.36-3.31 (m, 1H), 3.14-3.07 (m, 1H), 2.61 (s, 3H), 1.41 (d, J=6.9 Hz, 3H); ES-LCMS m/z 505.1 [M+H]⁺.

### Step 2: N-[(4-Methoxyphenyl)methyl]-N-methyl-3-(1-methylimidazol-4-yl)-4-[[(2R)-2-phenylpropyl]amino]benzenesulfonamide

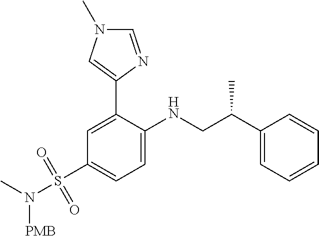

To a solution of 3-bromo-N-[(4-methoxyphenyl)methyl]-N-methyl-4-[[(2R)-2-phenylpropyl]amino]benzenesulfonamide (120 mg, 166.85 μmol, 70% purity, 1 eq) in DMF (3 mL) was added tributyl-(1-methylimidazol-4-yl)stannane (130.37 mg, 333.70 μmol, 95% purity, 2 eq) and Pd(dppf)Cl₂ (12.21 mg, 16.69 μmol, 0.1 eq). The mixture was stirred at 130° C. for 3 h under N₂ atmosphere. To the mixture was added water (10 mL) was added and extracted with EtOAc (20 mL×3). The combined organic layers were washed with brine (10 mL), dried over Na₂SO₄, filtered and concentrated under reduced pressure to give a residue which was purified by flash silica gel chromatography (from PE/EtOAc=1/0 to 1/1, TLC: PE/EtOAc=3/1, R_{f}=0.2) to yield N-[(4-methoxyphenyl)methyl]-N-methyl-3-(1-methylimidazol-4-yl)-4-[[(2R)-2-phenylpropyl]amino]benzenesulfonamide (80 mg, 150.60 μmol, 90.2% yield, 95.0% purity) as blue oil. ¹H NMR (400 MHZ, CDCl₃) δ ppm 8.59 (s, 1H), 7.75 (d, J=2.3 Hz, 1H), 7.53 (dd, J=2.2, 8.8 Hz, 1H), 7.39 (s, 1H), 7.35-7.28 (m, 5H), 7.25-7.16 (m, 5H), 6.84 (d, J=8.6 Hz, 2H), 6.70 (d, J=9.0 Hz, 1H), 4.01 (s, 2H), 3.79 (s, 3H), 3.73 (s, 3H), 3.46-3.37 (m, 2H), 3.18-3.12 (m, 1H), 2.51 (s, 3H), 1.42 (d, J=7.0 Hz, 3H); ES-LCMS m/z 505.3 [M+H]⁺.

### Step 3: N-Methyl-3-(1-methylimidazol-4-yl)-4-[[(2R)-2-phenylpropyl]amino]benzenesulfonamide

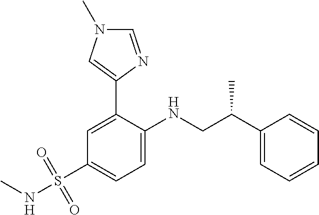 [SMILES: CNS(=O)(=O)c1ccc(NC[C@H](C)c2ccccc2)c(-c2cn(C)cn2)c1]

To a solution of N-[(4-methoxyphenyl)methyl]-N-methyl-3-(1-methylimidazol-4-yl)-4-[[(2R)-2-phenylpropyl]amino]benzenesulfonamide (80 mg, 150.60 μmol, 95% purity, 1 eq) in DCM (3 mL) was added TFA (1.46 g, 12.83 mmol, 950.00 μL, 85.20 eq). The mixture was stirred at 25° C. for 1 h. The reaction mixture was concentrated to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 40%-70%, 10 min), followed by lyophilization to yield N-methyl-3-(1-methylimidazol-4-yl)-4-[[(2R)-2-phenylpropyl]amino]benzenesulfonamide (21.87 mg, 56.88 μmol, 37.7% yield, 100.0% purity) as a white solid. ¹H NMR (400 MHZ, CDCl₃) δ ppm 8.62 (s, 1H), 7.80 (d, J=2.3 Hz, 1H), 7.54 (dd, J=2.3, 8.6 Hz, 1H), 7.37 (d, J=0.8 Hz, 1H), 7.34-7.31 (m, 1H), 7.30 (d, J=2.3 Hz, 3H), 7.25-7.20 (m, 1H), 7.18 (d, J=1.2 Hz, 1H), 6.67 (d, J=8.6 Hz, 1H), 4.17 (q, J=5.3 Hz, 1H), 3.72 (s, 3H), 3.40 (t, J=11.0 Hz, 2H), 3.17-3.11 (m, 1H), 2.60 (d, J=5.5 Hz, 3H), 1.41 (d, J=6.7 Hz, 3H); ES-LCMS m/z 385.2 [M+H]⁺.

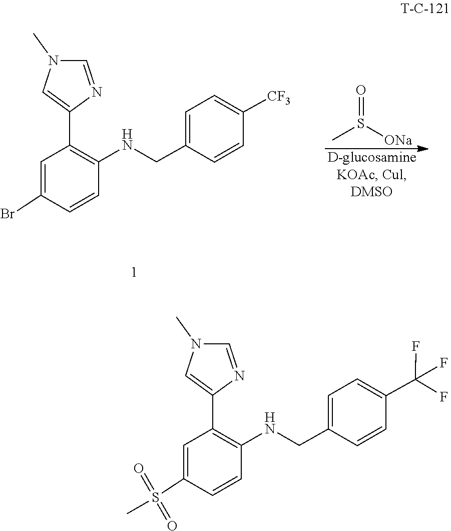

### Step 1: 2-(1-Methylimidazol-4-yl)-4-methylsulfonyl-N-[[4-(trifluoromethyl)phenyl]methyl]aniline

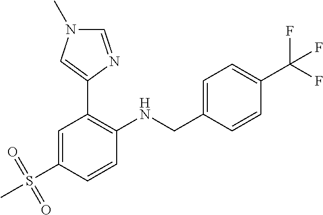 [SMILES: Cn1cnc(-c2cc(S(C)(=O)=O)ccc2NCc2ccc(C(F)(F)F)cc2)c1]

A mixture of 4-bromo-2-(1-methylimidazol-4-yl)-N-[[4-(trifluoromethyl)phenyl]methyl]aniline (100 mg, 236.45 μmol, 97%, 1 eq), methylsulfinyloxysodium (163.80 mg, 1.18 mmol, 5 eq, HCl), CuI (45.03 mg, 236.45 μmol, 1 eq), D-glucosamine (42.37 mg, 236.45 μmol, 1 eq) and KOAc (69.62 mg, 709.36 μmol, 3 eq) in DMSO (2.5 mL) and H₂O (2.5 mL) was degassed and purged with N₂ for 3 times and the mixture was stirred under N₂ atmosphere at 100° C. for 24 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 43%-73%, 10 min) and lyophilized to yield 2-(1-methylimidazol-4-yl)-4-methylsulfonyl-N-[[4-(trifluoromethyl)phenyl]methyl]aniline (11.7 mg, 28.58 μmol, 12.1% yield, 100.0% purity) as a white solid. ¹H NMR (400 MHZ, CDCl₃) δ ppm 9.37 (s, 1H), 7.93 (s, 1H), 7.63-7.45 (m, 6H), 6.57 (d, J=8.1 Hz, 1H), 4.62 (s, 2H), 3.79 (s, 3H), 3.02 (s, 3H); ES-LCMS m/z 410.2 [M+H]⁺.

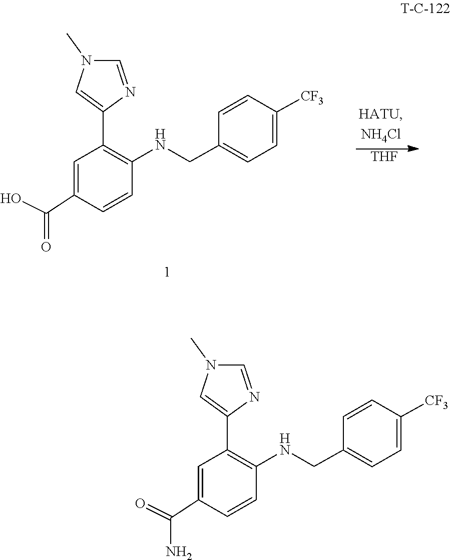 [SMILES: Cn1cnc(-c2cc(C(=O)O)ccc2NCc2ccc(C(F)(F)F)cc2)c1 ; Cn1cnc(-c2cc(C(N)=O)ccc2NCc2ccc(C(F)(F)F)cc2)c1 ; "THF"]

### Step 1: 3-(1-Methylimidazol-4-yl)-4-[[4-(trifluoromethyl)phenyl]methylamino]benzamide

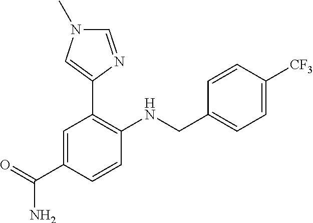 [SMILES: Cn1cnc(-c2cc(C(N)=O)ccc2NCc2ccc(C(F)(F)F)cc2)c1]

To a solution of 3-(1-methylimidazol-4-yl)-4-[[4-(trifluoromethyl)phenyl]methylamino]benzoic acid (50.00 mg, 133.21 μmol, 1 eq) in DMF (3 mL) was added NH₄Cl (50 mg, 934.75 μmol, 7.02 eq) and HATU (100 mg, 263.00 μmol, 1.97 eq). The mixture was stirred at 20° C. for 2 h. The reaction mixture was quenched with H₂O (20 mL) and extracted with EtOAc (20 mL×3). The combined organic layers dried over Na₂SO₄, filtered and concentrated under reduced pressure to yield a residue which was purified by preparative HPLC (column: Welch Xtimate C18 150×25 mm×5 μm; mobile phase: [water (10 mM NH₄HCO₃)-ACN]; B %: 35%-65%, 10 min), followed by lyophilization to yield 3-(1-methylimidazol-4-yl)-4-[[4-(trifluoromethyl)phenyl]methylamino]benzamide (27.25 mg, 72.79 μmol, 54.6% yield, 100.0%) as a white solid. ¹H NMR (400 MHZ, DMSO-d₆) δ ppm 9.20 (t, J=6.1 Hz, 1H), 8.03 (d, J=2.0 Hz, 1H), 7.77 (s, 1H), 7.72-7.67 (m, 3H), 7.62 (s, 1H), 7.57-7.53 (m, 1H), 7.55 (d, J=8.1 Hz, 1H), 7.50 (dd, J=1.7, 8.6 Hz, 1H), 6.93 (s, 1H), 6.50 (d, J=8.8 Hz, 1H), 4.62 (d, J=5.9 Hz, 2H), 3.75 (s, 3H); ES-LCMS m/z 375.3 [M+H]⁺.

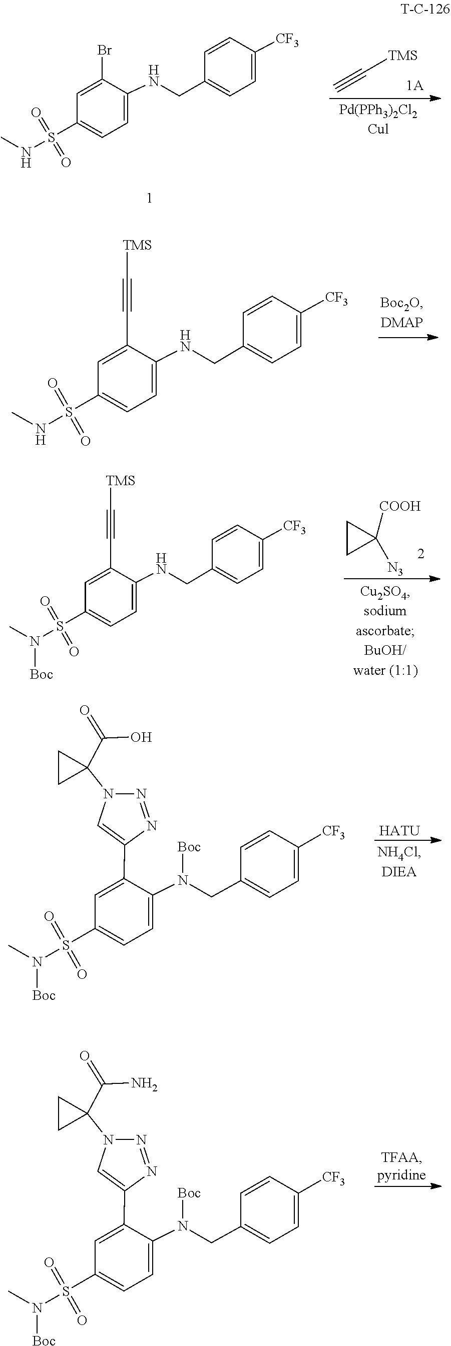

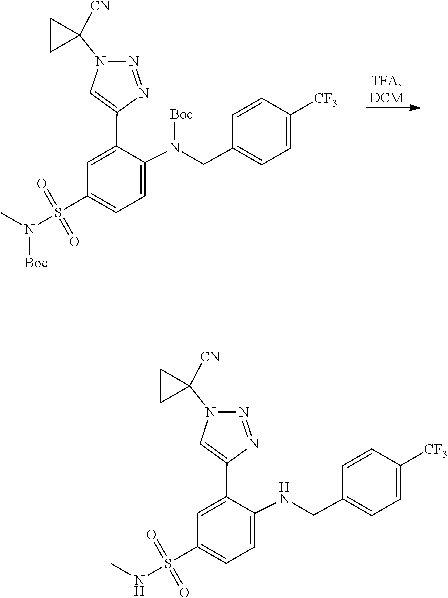

### Step 1: N-Methyl-4-[[4-(trifluoromethyl)phenyl]methylamino]-3-(2-trimethylsilylethynyl)benzenesulfonamide

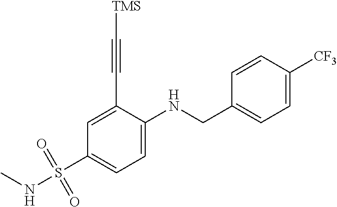 [SMILES: CNS(=O)(=O)c1ccc(NCc2ccc(C(F)(F)F)cc2)c(C#C[Si](C)(C)C)c1]

To a solution of 3-bromo-N-methyl-4-[[4-(trifluoromethyl)phenyl]methylamino]benzenesulfonamide (3.5 g, 7.09 mmol, 84% purity, 1 eq) and ethynyl(trimethyl)silane (2.09 g, 21.26 mmol, 2.95 mL, 3 eq) in TEA (30 mL) was added Pd(PPh₃)₂Cl₂ (248.75 mg, 354.40 μmol, 0.05 eq) and CuI (67.50 mg, 354.40 μmol, 0.05 eq). The mixture was stirred at 70° C. for 5 h. The reaction mixture was quenched by addition of water (50 mL) and extracted with EtOAc (50 mL×3). The combined organic layers were washed with brine (30 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.53) to yield N-methyl-4-[[4-(trifluoromethyl)phenyl]methylamino]-3-(2-trimethylsilylethynyl)benzenesulfonamide (3.0 g, 6.36 mmol, 89.7% yield, 93.3% purity) as a yellow oil. ¹H NMR (400 MHZ, CDCl₃) δ ppm 7.83 (d, J=2.2 Hz, 1H), 7.64 (d, J=8.1 Hz, 2H), 7.59 (dd, J=2.1, 8.7 Hz, 1H), 7.47 (d, J=8.1 Hz, 2H), 6.51 (d, J=8.8 Hz, 1H), 5.58 (t, J=5.5 Hz, 1H), 4.57 (d, J=5.9 Hz, 2H), 4.18 (q, J=5.5 Hz, 1H), 2.63 (d, J=5.4 Hz, 3H), 0.25 (s, 9H); ES-LCMS m/z 441.3 [M+H]⁺.

### Step 2: tert-Butyl N-[4-[tert-butoxycarbonyl(methyl)sulfamoyl]-2-(2-trimethylsilylethynyl)phenyl]-N-[[4-(trifluoromethyl)phenyl]methyl]carbamate

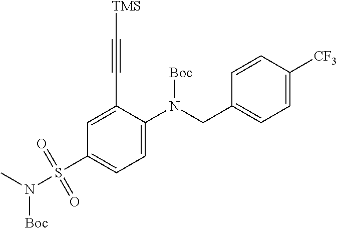 [SMILES: CN(C(=O)OC(C)(C)C)S(=O)(=O)c1ccc(N(Cc2ccc(C(F)(F)F)cc2)C(=O)OC(C)(C)C)c(C#C[Si](C)(C)C)c1]

To a solution of N-methyl-4-[[4-(trifluoromethyl)phenyl]methylamino]-3-(2-trimethylsilylethynyl)benzenesulfonamide (3 g, 6.36 mmol, 93.3% purity, 1 eq) in DCM (20 mL) was added DMAP (3.88 g, 31.78 mmol, 5 eq) and (Boc)₂O (13.87 g, 63.56 mmol, 14.60 mL, 10 eq) at 0° C. The mixture was stirred at 25° C. for 12 h. The reaction mixture was quenched by addition of water (50 mL) and extracted with EtOAc (30 mL×3). The combined organic layers were washed with brine (30 mL), dried over Na₂SO₄, filtered and concentrated under reduced pressure to yield a residue which was purified by flash silica gel chromatography (from PE/EtOAc=1/0 to 5/1, TLC: PE/EtOAc=3/1, R_{f}=0.49) to yield tert-butyl N-[4-[tert-butoxycarbonyl(methyl)sulfamoyl]-2-(2-trimethylsilylethynyl)phenyl]-N-[[4-(trifluoromethyl)phenyl]methyl]carbamate (1.9 g, 2.56 mmol, 40.3% yield, 86.5% purity) as colorless oil. ¹H NMR (400 MHZ, CDCl₃) δ ppm 7.97 (br s, 1H), 7.72 (d, J=6.1 Hz, 1H), 7.53 (d, J=7.8 Hz, 2H), 7.36 (d, J=8.1 Hz, 2H), 7.07 (br s, 1H), 4.88 (s, 2H), 3.36 (s, 3H), 1.40 (s, 9H), 1.34 (s, 9H), 0.25 (s, 9H); ES-LCMS m/z 658.3 [M+NH₄]⁺.

### Step 3: 1-[4-[5-[tert-Butoxycarbonyl(methyl)sulfamoyl]-2-[tert-butoxycarbonyl-[[4-(trifluoromethyl)phenyl]methyl]amino]phenyl]triazol-1-yl]cyclopropanecarboxylic acid

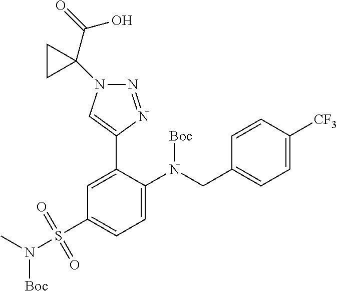 [SMILES: CN(C(=O)OC(C)(C)C)S(=O)(=O)c1ccc(N(Cc2ccc(C(F)(F)F)cc2)C(=O)OC(C)(C)C)c(-c2cn(C3(C(=O)O)CC3)nn2)c1]

To a solution of tert-butyl N-[4-[tert-butoxycarbonyl(methyl)sulfamoyl]-2-(2-trimethylsilylethynyl)phenyl]-N-[[4-(trifluoromethyl)phenyl]methyl]carbamate (1 g, 1.56 mmol, 1 eq) and 1-azidocyclopropanecarboxylic acid (595.06 mg, 4.68 mmol, 3 eq) in t-BuOH (15 mL) and H₂O (15 mL) was added sodium ascorbate (309.17 mg, 1.56 mmol, 1 eq) and CuSO₄ (249.09 mg, 1.56 mmol, 239.51 μL, 1 eq). The mixture was stirred at 25° C. for 12 h. The mixture was diluted with EtOAc (200 mL), washed with brine (20 mL×2), dried over Na₂SO₄, concentrated under reduced pressure to yield 1-[4-[5-[tert-butoxycarbonyl(methyl)sulfamoyl]-2-[tert-butoxycarbonyl-[[4-(trifluoromethyl)phenyl]methyl]amino]phenyl]triazol-1-yl]cyclopropanecarboxylic acid (1.44 g, 1.55 mmol, 99.4% yield, 75.0% purity) as a white solid. ¹H NMR (400 MHZ, CDCl₃) δ ppm 8.58 (br s, 1H), 7.80 (d, J=7.8 Hz, 1H), 7.55 (d, J=7.8 Hz, 3H), 7.39-7.29 (m, 2H), 7.03 (br s, 1H), 5.53 (s, 2H), 3.39 (s, 3H), 2.03 (s, 2H), 1.79 (d, J=8.8 Hz, 2H), 1.35 (s, 9H), 1.20 (s, 9H); ES-LCMS m/z 696.2 [M+H]⁺.

### Step 4 tert-Butyl N-[4-[tert-butoxycarbonyl(methyl)sulfamoyl]-2-[1-(1-carbamoylcyclopropyl)triazol-4-yl]phenyl]-N-[[4-(trifluoromethyl)phenyl]methyl]carbamate

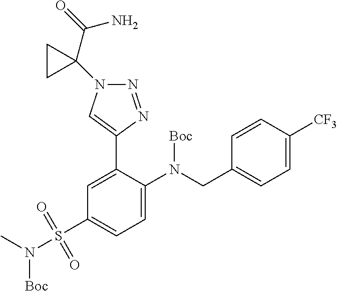 [SMILES: CN(C(=O)OC(C)(C)C)S(=O)(=O)c1ccc(N(Cc2ccc(C(F)(F)F)cc2)C(=O)OC(C)(C)C)c(-c2cn(C3(C(N)=O)CC3)nn2)c1]

To a solution of 1-[4-[5-[tert-butoxycarbonyl(methyl)sulfamoyl]-2-[tert-butoxycarbonyl-[[4-(trifluoromethyl)phenyl]methyl]amino]phenyl]triazol-1-yl]cyclopropanecarboxylic acid (1.2 g, 1.38 mmol, 80% purity, 1 eq) in DMF (15 mL) was added HATU (787.02 mg, 2.07 mmol, 1.5 eq), DIEA (356.68 mg, 2.76 mmol, 480.71 μL, 2 eq) and NH₄Cl (147.62 mg, 2.76 mmol, 2 eq). The mixture was stirred at 25° C. for 2 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to give a residue. The residue was purified by flash silica gel chromatography (from PE/EtOAc=1/0 to 1/1, TLC: PE/EtOAc=1/1, R_{f}=0.59) to yield tert-butyl N-[4-[tert-butoxycarbonyl(methyl)sulfamoyl]-2-[1-(1-carbamoylcyclopropyl)triazol-4-yl]phenyl]-N-[[4-(trifluoromethyl)phenyl]methyl]carbamate (1.1 g, 1.14 mmol, 83.0% yield, 72.3% purity) as colorless oil. ¹H NMR (400 MHZ, CDCl₃) δ ppm 8.57 (br s, 1H), 8.02 (s, 3H), 7.85 (d, J=8.3 Hz, 1H), 7.57 (d, J=8.1 Hz, 2H), 7.34 (d, J=8.1 Hz, 2H), 7.11 (br s, 1H), 5.50 (s, 2H), 3.40 (s, 3H), 1.99 (s, 2H), 1.61 (s, 9H), 1.51 (s, 2H), 1.37 (s, 9H); ES-LCMS m/z 695.2 [M+H]⁺.

### Step 5: tert-Butyl N-[4-[tert-butoxycarbonyl(methyl)sulfamoyl]-2-[1-(1-cyanocyclopropyl)triazol-4-yl]phenyl]-N-[[4-(trifluoromethyl)phenyl]methyl]carbamate

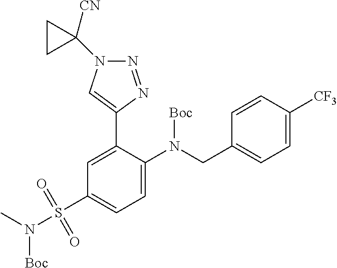 [SMILES: CN(C(=O)OC(C)(C)C)S(=O)(=O)c1ccc(N(Cc2ccc(C(F)(F)F)cc2)C(=O)OC(C)(C)C)c(-c2cn(C3(C#N)CC3)nn2)c1]

To a solution of tert-butyl N-[4-[tert-butoxycarbonyl(methyl)sulfamoyl]-2-[1-(1-carbamoylcyclopropyl)triazol-4-yl]phenyl]-N-[[4-(trifluoromethyl)phenyl]methyl]carbamate (400 mg, 416.23 μmol, 72.3% purity, 1 eq) in EtOAc (5 mL) was added TFAA (437.10 mg, 2.08 mmol, 289.47 μL, 5 eq) and pyridine (329.23 mg, 4.16 mmol, 335.95 μL, 10 eq). The mixture was stirred at 25° C. for 2 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield tert-butyl N-[4-[tert-butoxycarbonyl(methyl)sulfamoyl]-2-[1-(1-cyanocyclopropyl)triazol-4-yl]phenyl]-N-[[4-(trifluoromethyl)phenyl]methyl]carbamate (300 mg, 324.82 μmol, 78.0% yield, 73.3% purity) as colorless oil. ¹H NMR (400 MHZ, CDCl₃) δ ppm 8.63 (br s, 1H), 7.86 (d, J=8.1 Hz, 1H), 7.58 (d, J=8.1 Hz, 2H), 7.35 (dd, J=6.6, 11.0 Hz, 3H), 7.15 (br s, 1H), 5.07 (s, 1H), 4.48 (d, J=12.0 Hz, 1H), 3.40 (s, 3H), 3.05 (s, 4H), 1.37 (s, 9H), 1.15 (s, 9H); ES-LCMS m/z 696.2 [M+H]⁺.

### Step 6: N-Cyclopropyl-5-(methylsulfamoyl)-2-[[4-(trifluoromethyl)phenyl]methylamino]benzamide

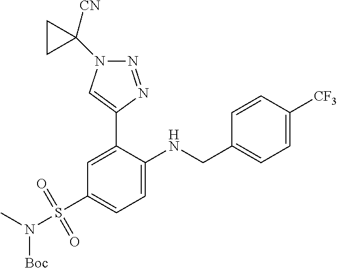 [SMILES: CN(C(=O)OC(C)(C)C)S(=O)(=O)c1ccc(NCc2ccc(C(F)(F)F)cc2)c(-c2cn(C3(C#N)CC3)nn2)c1]

To a solution of tert-butyl N-[4-[tert-butoxycarbonyl(methyl)sulfamoyl]-2-[1-(1-cyanocyclopropyl)triazol-4-yl]phenyl]-N-[[4-(trifluoromethyl)phenyl]methyl]carbamate (300 mg, 324.82 μmol, 73.3% purity, 1 eq) in DCM (5 mL) was added TFA (1.54 g, 13.51 mmol, 1 mL, 41.58 eq). The mixture was stirred at 25° C. for 1 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 45%-75%, 10 min), followed by lyophilization to yield 3-[1-(1-cyanocyclopropyl)triazol-4-yl]-N-methyl-4-[[4-(trifluoromethyl)phenyl]methylamino]benzenesulfonamide (91.24 mg, 191.49 μmol, 59.0% yield, 100.0% purity) as a white solid. ¹H NMR (400 MHZ, DMSO-d₆) δ ppm 9.12 (s, 1H), 8.19 (t, J=6.0 Hz, 1H), 7.91 (d, J=2.2 Hz, 1H), 7.71 (d, J=8.3 Hz, 2H), 7.58 (d, J=8.1 Hz, 2H), 7.47 (dd, J=2.0, 8.8 Hz, 1H), 7.05 (q, J=4.7 Hz, 1H), 6.74 (d, J=9.0 Hz, 1H), 4.69 (d, J=5.6 Hz, 2H), 2.36 (d, J=5.1 Hz, 3H), 2.11 (d, J=10.0 Hz, 4H); ES-LCMS m/z 477.2 [M+H]⁺.

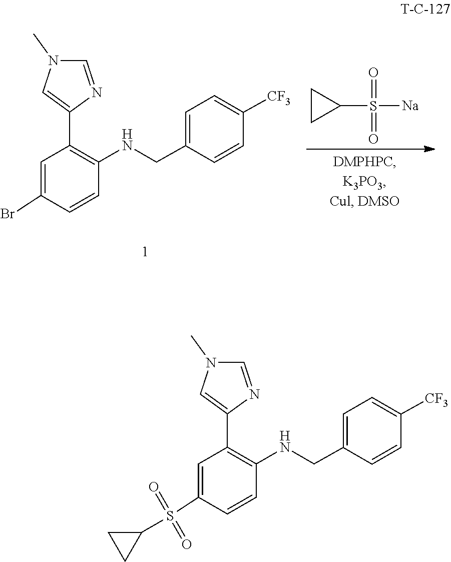

### Step 1: 4-Cyclopropylsulfonyl-2-(1-methylimidazol-4-yl)-N-[[4-(trifluoromethyl)phenyl]methyl]aniline

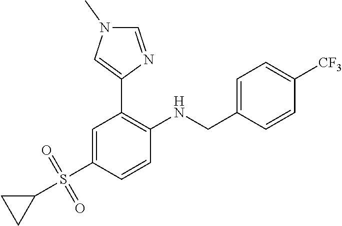 [SMILES: Cn1cnc(-c2cc(S(=O)(=O)C3CC3)ccc2NCc2ccc(C(F)(F)F)cc2)c1]

To a solution of 4-bromo-2-(1-methylimidazol-4-yl)-N-[[4-(trifluoromethyl)phenyl]methyl]aniline (100 mg, 236.45 μmol, 97%, 1 eq) and cyclopropylsulfinyloxysodium (302.96 mg, 2.36 mmol, 10 eq) in DMSO (2.5 mL) and H₂O (2.5 mL) was added CuI (45.03 mg, 236.45 μmol, 1 eq) and D-glucosamine (42.37 mg, 236.45 μmol, 1 eq) and KOAc (92.82 mg, 945.81 μmol, 4 eq). The mixture was stirred at 110° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by preparative HPLC (column: Welch Xtimate C18 150*25 mm*5 μm; mobile phase: [water (10 mM NH₄HCO₃)-ACN]; B %: 42%-72%, 10 min), followed by lyophilization to yield 4-cyclopropylsulfonyl-2-(1-methylimidazol-4-yl)-N-[[4-(trifluoromethyl)phenyl]methyl]aniline (15.99 mg, 35.69 μmol, 15.0% yield, 97.2% purity) as a white solid. ¹H NMR (400 MHZ, CDCl₃) δ ppm 9.31 (s, 1H), 7.88 (d, J=2.0 Hz, 1H), 7.60 (d, J=8.1 Hz, 2H), 7.54-7.47 (m, 4H), 7.33 (s, 1H), 6.56 (d, J=8.8 Hz, 1H), 4.61 (d, J=5.6 Hz, 2H), 3.79 (s, 3H), 2.47-2.39 (m, 1H), 1.32-1.26 (m, 2H), 1.00-0.94 (m, 2H); ES-LCMS m/z 436.0 [M+H]⁺.

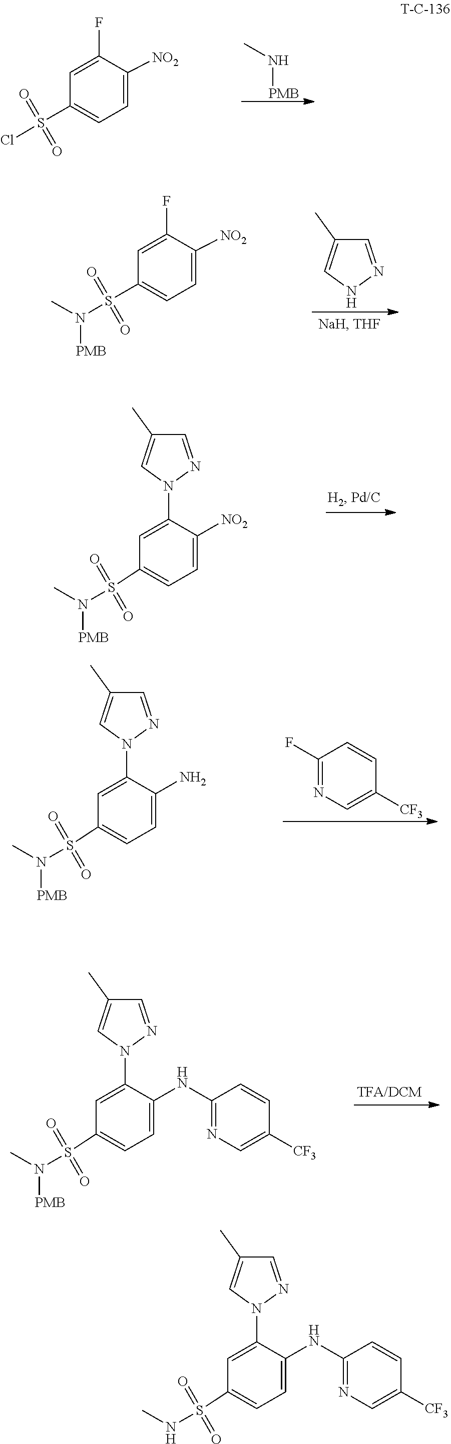

### Step 1: 3-Fluoro-N-[(4-methoxyphenyl)methyl]-N-methyl-4-nitro-benzenesulfonamide

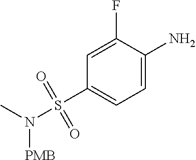

To a solution of 3-fluoro-4-nitro-benzenesulfonyl chloride (950 mg, 3.96 mmol, 1 eq) in THF (14 mL) was added 1-(4-methoxyphenyl)-N-methyl-methanamine (608.00 mg, 4.02 mmol, 1.01 eq) and DIEA (1.54 g, 11.89 mmol, 2.07 mL, 3 eq) at −20° C. The mixture was stirred at −20° C. for 1 h under N₂ atmosphere. TLC (PE/EtOAc=3/1, R_{f}=0.40) indicated starting material was consumed completely and one new spot formed. The reaction mixture was quenched by 2 M HCl at −10° C. until pH=2, diluted with water (20 mL) and extracted with EtOAc (20 mL×3). The combined organic layers were washed with 1 M HCl (20 mL×3), dried over Na₂SO₄, filtered and concentrated under reduced pressure to yield 3-fluoro-N-[(4-methoxyphenyl)methyl]-N-methyl-4-nitro-benzenesulfonamide (1.33 g, 3.57 mmol, 89.9% yield, 95.0% purity) as a yellow solid, which was used in the next step without further purification. ¹H NMR (400 MHZ, CDCl₃) δ ppm 8.21 (dd, J=7.0, 8.7 Hz, 1H), 7.75 (d, J=2.0 Hz, 1H), 7.74-7.72 (m, 1H), 7.21 (d, J=8.6 Hz, 2H), 6.88 (d, J=8.8 Hz, 2H), 4.18 (s, 2H), 3.82 (s, 3H), 2.69 (s, 3H).

### Step 2: N-[(4-Methoxyphenyl)methyl]-N-methyl-3-(4-methylpyrazol-1-yl)-4-nitro-benzenesulfonamide

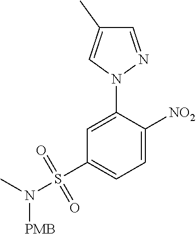

To a solution of 4-methyl-1H-pyrazole (292.75 mg, 3.57 mmol, 287.01 μL, 1 eq) in THF (15 mL) was added NaH (855.68 mg, 21.39 mmol, 60% purity, 6 eq) at 0° C. The mixture was stirred at 0° C. for 30 min. 3-Fluoro-N-[(4-methoxyphenyl)methyl]-N-methyl-4-nitro-benzenesulfonamide (1.33 g, 3.57 mmol, 95% purity, 1 eq) was added and the mixture was stirred at 25° C. for 12 h. The reaction mixture was quenched by water (30 mL) and extracted with EtOAc (30 mL×3). The combined organic layers were dried over Na₂SO₄, filtered and concentrated under reduced pressure to yield a residue which was purified by preparative TLC (PE/EtOAc=2/1, R_{f}=0.40) to yield N-[(4-methoxyphenyl)methyl]-N-methyl-3-(4-methylpyrazol-1-yl)-4-nitro-benzenesulfonamide (360 mg, 821.23 μmol, 23.0% yield, 95.0% purity) as a yellow solid. ¹H NMR (400 MHZ, CDCl₃) δ ppm 7.97 (d, J=1.7 Hz, 1H), 7.95-7.89 (m, 1H), 7.87-7.82 (m, 1H), 7.60 (s, 1H), 7.55 (s, 1H), 7.23 (d, J=8.6 Hz, 2H), 6.88 (d, J=8.8 Hz, 2H), 4.19 (s, 2H), 3.81 (s, 3H), 2.70 (s, 3H), 2.18 (s, 3H); ES-LCMS m/z 417.4 [M+H]⁺.

### Step 3: 4-Amino-N-[(4-methoxyphenyl)methyl]-N-methyl-3-(4-methylpyrazol-1-yl)benzenesulfonamide

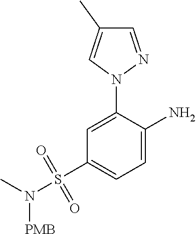

To a solution of N-[(4-methoxyphenyl)methyl]-N-methyl-3-(4-methylpyrazol-1-yl)-4-nitro-benzenesulfonamide (310 mg, 707.17 μmol, 95% purity, 1 eq) in MeOH (80 mL) was added Pd/C (294.50 mg, 277.83 μmol, 10% purity, 3.93e-1 eq). The mixture was stirred at 25° C. for 2 h. TLC (PE/EtOAc=2/1, R_{f}=0.50) indicated starting material was consumed completely and one new spot formed. The mixture was filtered and concentrated under reduced pressure to yield 4-amino-N-[(4-methoxyphenyl)methyl]-N-methyl-3-(4-methylpyrazol-1-yl)benzenesulfonamide (300 mg, 698.64 μmol, 98.8% yield, 90.0% purity) as a brown solid, which was used in the next step without further purification. ¹H NMR (400 MHZ, DMSO-d₆) δ ppm 8.03 (s, 1H), 7.62 (s, 1H), 7.53 (d, J=2.2 Hz, 1H), 7.50 (dd, J=2.0, 8.6 Hz, 1H), 7.23 (d, J=8.6 Hz, 2H), 7.00 (d, J=8.6 Hz, 1H), 6.91 (d, J=8.6 Hz, 2H), 6.50 (s, 2H), 4.00 (s, 2H), 3.74 (s, 3H), 2.46 (s, 3H), 2.15-2.07 (m, 3H); ES-LCMS m/z 387.2 [M+H]⁺.

### Step 4: N-[(4-Methoxyphenyl)methyl]-N-methyl-3-(4-methylpyrazol-1-yl)-4-[[5-(trifluoromethyl)-2-pyridyl]amino]benzenesulfonamide

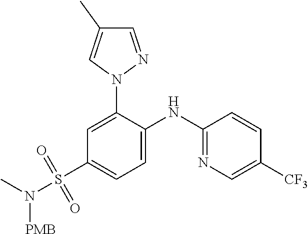

To a solution of 4-amino-N-[(4-methoxyphenyl)methyl]-N-methyl-3-(4-methylpyrazol-1-yl)benzenesulfonamide (200 mg, 465.76 μmol, 90% purity, 1 eq) and 2-fluoro-5-(trifluoromethyl)pyridine (76.89 mg, 465.76 μmol, 1 eq) in DMF (2 mL) was added K₂CO₃ (193.11 mg, 1.40 mmol, 3 eq). The mixture was stirred at 130° C. for 3 h. TLC (PE/EtOAc=3/1, R_{f}=0.60) indicated 40% of starting material remained and one major new spot with lower polarity was detected. The reaction mixture was quenched by water (10 mL) and extracted with EtOAc (10 mL×3). The combined organic layers were dried over Na₂SO₄, filtered and concentrated under reduced pressure to yield a residue which was purified by flash silica gel chromatography (from PE/EtOAc=100/1 to 4/1, TLC: PE/EtOAc=3/1, R_{f}=0.60) to yield N-[(4-methoxyphenyl)methyl]-N-methyl-3-(4-methylpyrazol-1-yl)-4-[[5-(trifluoromethyl)-2-pyridyl]amino]benzenesulfonamide (110 mg, 186.25 μmol, 40.0% yield, 90.0% purity) as a yellow solid. ¹H NMR (400 MHZ, CDCl₃) δ ppm 10.53 (s, 1H), 8.87 (d, J=9.5 Hz, 1H), 8.55 (s, 1H), 7.78-7.74 (m, 3H), 7.71-7.67 (m, 2H), 7.24 (d, J=8.4 Hz, 2H), 6.90-6.85 (m, 3H), 4.13 (s, 2H), 3.82-3.80 (m, 3H), 2.67-2.57 (m, 3H), 2.21 (s, 3H); ES-LCMS m/z 532.6 [M+H]⁺.

### Step 5 N-Methyl-3-(4-methylpyrazol-1-yl)-4-[[5-(trifluoromethyl)-2-pyridyl]amino]benzenesulfonamide

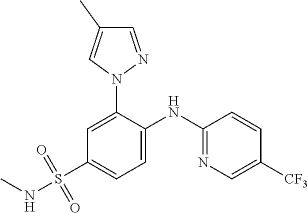 [SMILES: CNS(=O)(=O)c1ccc(Nc2ccc(C(F)(F)F)cn2)c(-n2cc(C)cn2)c1]

A solution of N-[(4-methoxyphenyl)methyl]-N-methyl-3-(4-methylpyrazol-1-yl)-4-[[5-(trifluoromethyl)-2-pyridyl]amino]benzenesulfonamide (110 mg, 186.25 μmol, 90% purity, 1 eq) in DCM (1 mL) and TFA (0.2 mL) was stirred at 25° C. for 12 h. The mixture was concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 48%-78%, 10 min), followed by lyophilization to yield N-methyl-3-(4-methylpyrazol-1-yl)-4-[[5-(trifluoromethyl)-2-pyridyl]amino]benzenesulfonamide (15.09 mg, 36.68 μmol, 19.7% yield, 100.0% purity) as a white solid. ¹H NMR (400 MHZ, CDCl₃) δ ppm 10.51 (s, 1H), 8.84 (d, J=8.9 Hz, 1H), 8.54 (s, 1H), 7.82 (d, J=2.0 Hz, 1H), 7.79 (dd, J=2.0, 8.9 Hz, 1H), 7.75 (dd, J=2.4, 8.6 Hz, 1H), 7.70 (d, J=13.9 Hz, 2H), 6.85 (d, J=8.7 Hz, 1H), 4.44-4.21 (m, 1H), 2.71 (d, J=5.5 Hz, 3H), 2.21 (s, 3H); ES-LCMS m/z 412.2 [M+H]⁺.

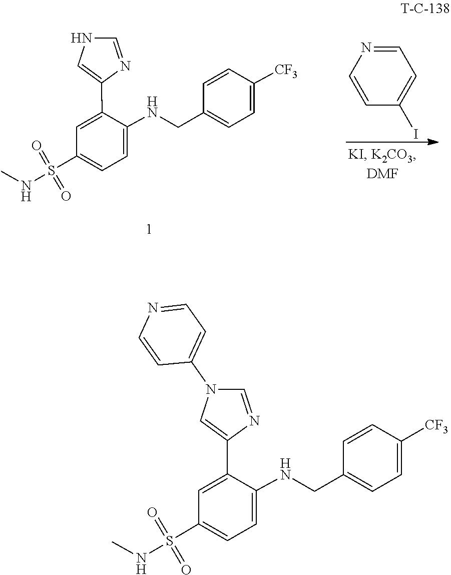 [SMILES: CNS(=O)(=O)c1ccc(NCc2ccc(C(F)(F)F)cc2)c(-c2c[nH]cn2)c1 ; Ic1ccncc1 ; CNS(=O)(=O)c1ccc(NCc2ccc(C(F)(F)F)cc2)c(-c2cn(-c3ccncc3)cn2)c1]

### Step 1: N-Methyl-3-[1-(4-pyridyl)imidazol-4-yl]-4-[[4-(trifluoromethyl)phenyl]methylamino]benzenesulfonamide

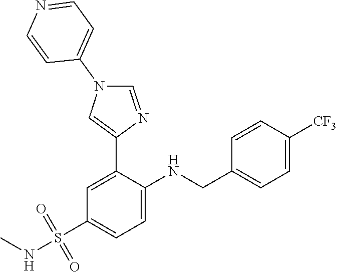 [SMILES: CNS(=O)(=O)c1ccc(NCc2ccc(C(F)(F)F)cc2)c(-c2cn(-c3ccncc3)cn2)c1]

To a solution of 3-(1H-imidazol-4-yl)-N-methyl-4-[[4-(trifluoromethyl)phenyl]methylamino]benzenesulfonamide (150 mg, 347.21 μmol, 95.0% purity, 1 eq) and 4-iodopyridine (92.53 mg, 451.37 μmol, 1.3 eq) in DMF (4 mL) was added KI (57.64 mg, 347.21 μmol, 1 eq) and K₂CO₃ (110.37 mg, 798.59 μmol, 2.3 eq). The mixture was bubbled with N₂ for 3 min and stirred under microwave (0 Bar) at 130° C. for 3 h. The reaction mixture was filtered and concentrated under reduced pressure to yield a residue which was purified by preparative HPLC (column: Phenomenex Synergi C18 150*30 mm*4 μm; mobile phase: [water (0.05% HCl)-ACN]; B %: 27%-57%, 10 min) to yield N-methyl-3-[1-(4-pyridyl)imidazol-4-yl]-4-[[4-(trifluoromethyl)phenyl]methylamino]benzenesulfonamide (12.92 mg, 23.81 μmol, 6.9% yield, 96.6% purity, HCl) as a yellow solid. ¹H NMR (400 MHZ, DMSO-d₆) δ ppm 9.01-8.89 (m, 3H), 8.74 (s, 1H), 8.29 (s, 2H), 8.03 (d, J=2.2 Hz, 1H), 7.73 (d, J=8.3 Hz, 2H), 7.60 (d, J=7.8 Hz, 2H), 7.44 (dd, J=2.2, 8.8 Hz, 1H), 7.08 (s, 1H), 6.72 (d, J=8.8 Hz, 1H), 4.68 (s, 2H), 2.38 (s, 3H); ES-LCMS m/z 488.1 [M+H]⁺.

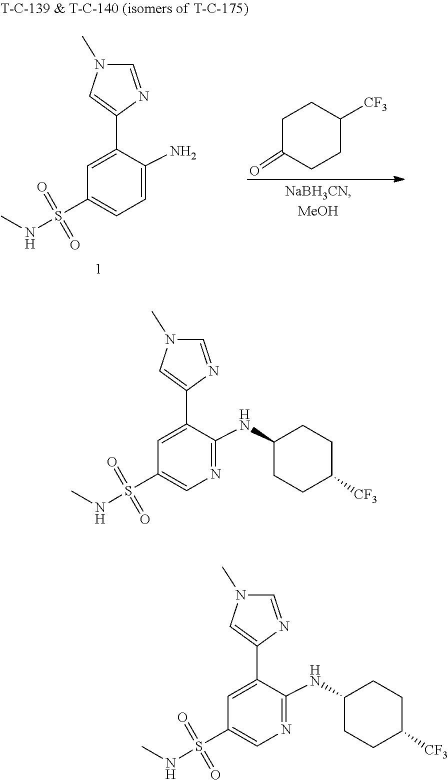 [SMILES: CNS(=O)(=O)c1ccc(N)c(-c2cn(C)cn2)c1 ; O=C1CCC(C(F)(F)F)CC1 ; CNS(=O)(=O)c1cnc(N[C@H]2CC[C@H](C(F)(F)F)CC2)c(-c2cn(C)cn2)c1 ; CNS(=O)(=O)c1cnc(N[C@H]2CC[C@@H](C(F)(F)F)CC2)c(-c2cn(C)cn2)c1]

### Step 1: N-Methyl-3-(1-methyl-1H-imidazol-4-yl)-4-(((1r,4r)-4-(trifluoromethyl)cyclohexyl)amino)benzenesulfonamide and N-methyl-3-(1-methyl-1H-imidazol-4-yl)-4-(((1s,4s)-4-(trifluoromethyl)cyclohexyl)amino)benzenesulfonamide

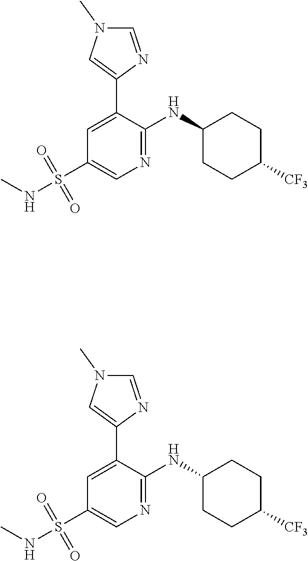 [SMILES: CNS(=O)(=O)c1cnc(N[C@H]2CC[C@H](C(F)(F)F)CC2)c(-c2cn(C)cn2)c1 ; CNS(=O)(=O)c1cnc(N[C@H]2CC[C@@H](C(F)(F)F)CC2)c(-c2cn(C)cn2)c1]

To a solution of 4-amino-N-methyl-3-(1-methylimidazol-4-yl)benzenesulfonamide (100 mg, 337.94 μmol, 90% purity, 1 eq) in MeOH (5 mL) was added 4-(trifluoromethyl)cyclohexanone (168.44 mg, 1.01 mmol, 3 eq), followed by 1 drop of AcOH. The mixture was stirred at 50° C. for 2 h. NaBH₃CN (63.71 mg, 1.01 mmol, 3 eq) was added and the mixture was stirred at 50° C. for 16 h. The solvent was removed to yield a residue which was purified by preparative TLC (PE/EtOAc=1/1, P1 (R_{f}=0.36); P2 (R_{f}=0.52)) to yield a product which was dissolved in ACN (5 mL) and H₂O (20 mL) and lyophilized to yield N-methyl-3-(1-methyl-1H-imidazol-4-yl)-4-(((1r,4r)-4-(trifluoromethyl)cyclohexyl)amino)benzenesulfonamide (33.97 mg, 79.94 μmol, 23.7% yield, 98.0% purity) as an off-white solid. ¹H NMR (400 MHZ, DMSO-d₆) δ ppm 8.72 (d, J=7.6 Hz, 1H), 7.82-7.70 (m, 2H), 7.62 (s, 1H), 7.38 (dd, J=2.0, 8.8 Hz, 1H), 6.98 (q, J=5.1 Hz, 1H), 6.83 (d, J=9.0 Hz, 1H), 3.72 (s, 3H), 3.51-3.39 (m, 1H), 2.35 (d, J=5.1 Hz, 4H), 2.13 (d, J=11.5 Hz, 2H), 1.92 (d, J=11.5 Hz, 2H), 1.55-1.40 (m, 2H), 1.34-1.20 (m, 2H); ES-LCMS m/z 417.2 [M+H]⁺ and N-methyl-3-(1-methyl-1H-imidazol-4-yl)-4-(((1s,4s)-4-(trifluoromethyl)cyclohexyl)amino)benzenesulfonamide (19.83 mg, 47.62 μmol, 14.1% yield, 100.0% purity) as an off-white solid. ¹H NMR (400 MHZ, DMSO-d₆) δ ppm 9.28 (d, J=7.8 Hz, 1H), 7.84-7.74 (m, 2H), 7.67 (d, J=1.0 Hz, 1H), 7.39 (dd, J=2.0, 8.8 Hz, 1H), 6.99 (q, J=5.1 Hz, 1H), 6.78 (d, J=9.0 Hz, 1H), 3.91 (s, 1H), 3.73 (s, 3H), 2.36 (d, J=5.1 Hz, 4H), 1.87 (d, J=12.5 Hz, 2H), 1.78-1.51 (m, 6H); ES-LCMS m/z 417.2 [M+H]⁺.

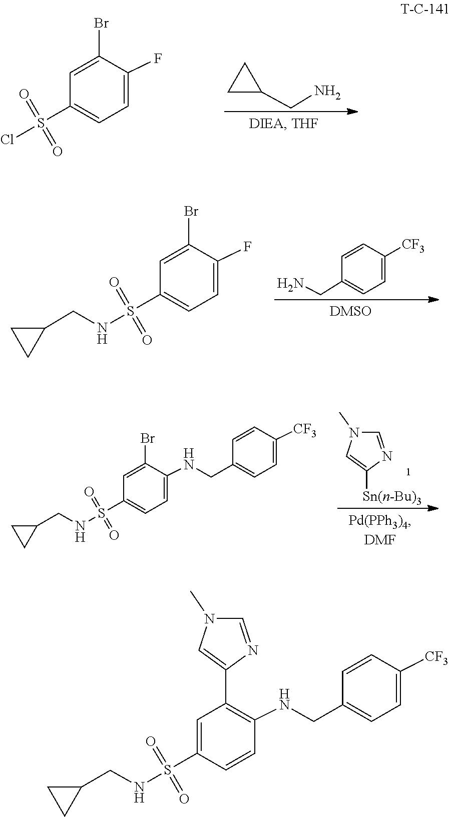

### Step 1: 3-Bromo-N-(cyclopropylmethyl)-4-fluoro-benzenesulfonamide

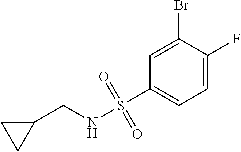 [SMILES: O=S(=O)(NCC1CC1)c1ccc(F)c(Br)c1]

To a solution of 3-bromo-4-fluoro-benzenesulfonyl chloride (0.5 g, 1.83 mmol, 1 eq) in THF (10 mL) was added cyclopropylmethanamine (260.03 mg, 3.66 mmol, 2 eq) and DIEA (708.79 mg, 5.48 mmol, 955.25 μL, 3 eq). The mixture was stirred at 25° C. for 2 h. TLC (PE/EtOAc=5/1, R_{f}=0.40) showed the starting materials was consumed completely and one new spot was detected. The mixture was adjusted pH=7-8 by HCl (1 N), added water (50 mL) and extracted with EtOAc (40 mL×3). The combined organic layers were washed with brine (60 mL), dried over Na₂SO₄, filtered and concentrated to yield a residue which was purified by silica gel column chromatography (from pure PE to PE/EtOAc=5/1, TLC: PE/EtOAc=5/1, R_{f}=0.40) to yield 3-bromo-N-(cyclopropylmethyl)-4-fluoro-benzenesulfonamide (230 mg, 716.50 μmol, 39.2% yield, 96.0% purity) as a white solid. ¹H NMR (500 MHz, CDCl₃) δ ppm 8.10 (dd, J=2.3, 6.3 Hz, 1H), 7.81 (m, 1H), 7.24 (t, J=8.5 Hz, 1H), 4.89-4.40 (m, 1H), 2.86 (t, J=6.5 Hz, 2H), 0.91-0.87 (m, 1H), 0.52-0.49 (m, 2H), 0.15-0.12 (m, 2H); ES-LCMS m/z 308.1, 310.1 [M+H]⁺.

### Step 2: 3-Bromo-N-(cyclopropylmethyl)-4-((4-(trifluoromethyl)benzyl)amino)benzenesulfonamide

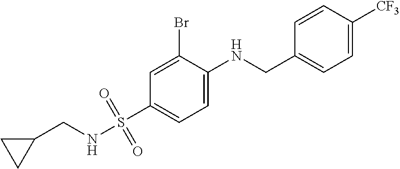 [SMILES: O=S(=O)(NCC1CC1)c1ccc(NCc2ccc(C(F)(F)F)cc2)c(Br)c1]

To a solution of 3-bromo-N-(cyclopropylmethyl)-4-fluoro-benzenesulfonamide (160 mg, 498.43 μmol, 96%, 1 eq) in DMSO (5 mL) was added [4-(trifluoromethyl)phenyl]methanamine (174.60 mg, 996.86 μmol, 141.95 μL, 2 eq). The mixture was stirred at 130° C. for 12 h. The reaction mixture was partitioned between water (50 mL) and EtOAc (100 mL×3). The organic phase was separated, washed with brine (50 mL×3), dried over Na₂SO₄, filtered and concentrated under reduced pressure to yield a residue which was purified on silica gel column chromatography (from pure PE to PE/EtOAc=5/1, TLC: PE/EtOAc=5/1, R_{f}=0.55) to yield 3-bromo-N-(cyclopropylmethyl)-4-((4-(trifluoromethyl)benzyl)amino)benzenesulfonamide (50 mg, 52.88 μmol, 10.6% yield, 49.0% purity) as white oil. ¹H NMR (500 MHZ, CDCl₃) δ ppm 7.97 (d, J=2.0 Hz, 1H), 7.64 (d, J=8.1 Hz, 2H), 7.60 (dd, J=2.0, 8.7 Hz, 1H), 7.46 (d, J=7.9 Hz, 2H), 6.53 (d, J=8.7 Hz, 1H), 5.33 (s, 1H), 4.56 (d, J=5.0 Hz, 2H), 4.42 (t, J=5.9 Hz, 1H), 2.81 (t, J=6.5 Hz, 2H), 0.91-0.87 (m, 1H), 0.51-0.46 (m, 2H), 0.11 (q, J=5.0 Hz, 2H); ES-LCMS m/z 463.1 [M+H]⁺.

### Step 3: N-(Cyclopropylmethyl)-3-(1-methyl-1H-imidazol-4-yl)-4-((4-(trifluoromethyl)benzyl)amino)benzenesulfonamide

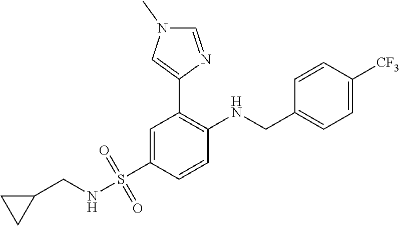 [SMILES: Cn1cnc(-c2cc(S(=O)(=O)NCC3CC3)ccc2NCc2ccc(C(F)(F)F)cc2)c1]

To a solution of 3-bromo-N-(cyclopropylmethyl)-4-((4-(trifluoromethyl)benzyl)amino)benzenesulfonamide (50 mg, 52.88 μmol, 49%, 1 eq) and tributyl-(1-methylimidazol-4-yl)stannane (43.61 mg, 105.76 μmol, 90%, 2 eq) in DMF (5 mL) was added Pd(PPh₃)+(3.06 mg, 2.64 μmol, 0.05 eq) under N₂ atmosphere. The mixture was stirred under N₂ atmosphere at 130° C. for 12 h. The mixture was diluted with water (80 mL) and extracted with EtOAc (50 mL×3). The combined organic layers were washed with brine (50 mL), dried over Na₂SO₄, filtered and concentrated to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 50%-80%, 10 min) to yield N-(cyclopropylmethyl)-3-(1-methyl-1H-imidazol-4-yl)-4-((4-(trifluoromethyl)benzyl)amino)benzenesulfonamide (8.75 mg, 18.84 μmol, 35.6% yield, 100.0% purity) as a white solid. ¹H NMR (400 MHZ, CDCl₃) δ ppm 9.22 (s, 1H), 7.87 (d, J=2.3 Hz, 1H), 7.59 (d, J=8.2 Hz, 2H), 7.49 (d, J=5.9 Hz, 4H), 7.31 (d, J=1.2 Hz, 1H), 6.54 (d, J=8.6 Hz, 1H), 4.60 (d, J=5.5 Hz, 2H), 4.32-4.24 (m, 1H), 3.79 (s, 3H), 2.82-2.74 (m, 2H), 0.88 (s, 1H), 0.49-0.40 (m, 2H), 0.11-0.02 (m, 2H); ES-LCMS m/z 465.2 [M+H]⁺.

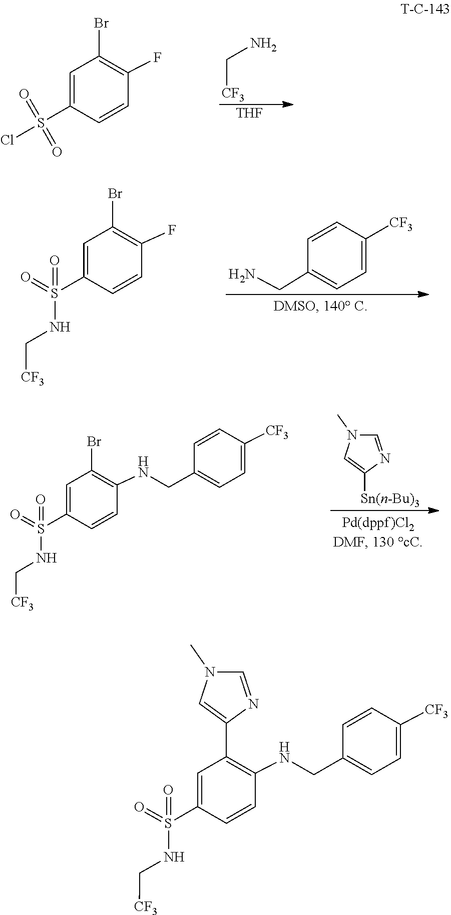

### Step 1: 3-Bromo-4-fluoro-N-(2,2,2-trifluoroethyl)benzenesulfonamide

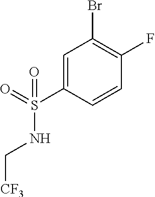 [SMILES: O=S(=O)(NCC(F)(F)F)c1ccc(F)c(Br)c1]

To a solution of 3-bromo-4-fluoro-benzenesulfonyl chloride (300 mg, 1.04 mmol, 95% purity, 1 eq) in THF (5 mL) was added a solution of 2,2,2-trifluoroethanamine (206.43 mg, 2.08 mmol, 163.84 μL, 2 eq) in THF (3 mL). The mixture was stirred at 25° C. for 16 h. The mixture was diluted with water (15 mL) and extracted with EtOAc (15 mL×3). The combined organic layers were washed with brine (15 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.3) to yield 3-bromo-4-fluoro-N-(2,2,2-trifluoroethyl)benzenesulfonamide (300 mg, 847.96 μmol, 81.3% yield, 95.0% purity) as a white solid. ¹H NMR (400 MHZ, CDCl₃) δ ppm 8.09 (dd, J=2.3, 6.3 Hz, 1H), 7.81 (ddd, J=2.3, 4.3, 8.6 Hz, 1H), 7.28-7.24 (m, 1H), 5.18 (s, 1H), 3.75-3.66 (m, 2H).

### Step 2: 3-Bromo-N-(2,2,2-trifluoroethyl)-4-[[4-(trifluoromethyl)phenyl]methylamino]benzenesulfonamide

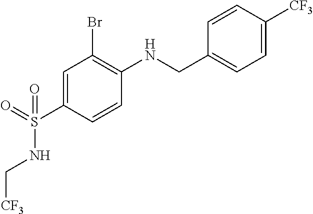 [SMILES: O=S(=O)(NCC(F)(F)F)c1ccc(NCc2ccc(C(F)(F)F)cc2)c(Br)c1]

To a solution of 3-bromo-4-fluoro-N-(2,2,2-trifluoroethyl)benzenesulfonamide (200 mg, 595.06 μmol, 1 eq) in DMSO (3 mL) was added [4-(trifluoromethyl)phenyl]methanamine (208.45 mg, 1.19 mmol, 169.47 μL, 2 eq). The mixture was stirred at 140° C. for 16 h. The mixture was diluted with water (20 mL) and extracted with EtOAc (20 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 1/1, TLC: PE/EtOAc=3/1, R_{f}=0.2) to yield 3-bromo-N-(2,2,2-trifluoroethyl)-4-[[4-(trifluoromethyl)phenyl]methylamino]benzenesulfonamide (250 mg, 458.02 μmol, 76.9% yield, 90.0% purity) as a white solid. ¹H NMR (400 MHZ, CDCl₃) δ ppm 7.96 (d, J=2.0 Hz, 1H), 7.64 (d, J=8.2 Hz, 2H), 7.60 (dd, J=2.0, 8.6 Hz, 1H), 7.44 (d, J=8.2 Hz, 2H), 6.53 (d, J=8.6 Hz, 1H), 5.39 (s, 1H), 4.80 (s, 1H), 4.56 (d, J=5.9 Hz, 2H), 3.63 (d, J=8.6 Hz, 2H); ES-LCMS m/z 493.0 [M+H]⁺.

### Step 3: 3-(1-Methylimidazol-4-yl)-N-(2,2,2-trifluoroethyl)-4-[[4-(trifluoromethyl)phenyl]methylamino]benzenesulfonamide

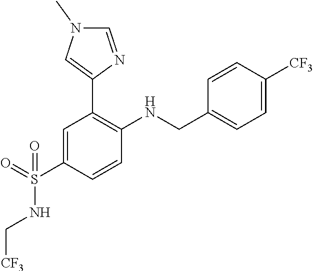 [SMILES: Cn1cnc(-c2cc(S(=O)(=O)NCC(F)(F)F)ccc2NCc2ccc(C(F)(F)F)cc2)c1]

To a solution of 3-bromo-N-(2,2,2-trifluoroethyl)-4-[[4-(trifluoromethyl)phenyl]methylamino]benzenesulfonamide (250 mg, 458.02 μmol, 90% purity, 1 eq) in DMF (3 mL) was added tributyl-(1-methylimidazol-4-yl)stannane (357.88 mg, 916.04 μmol, 95% purity, 2 eq) and Pd(dppf)Cl₂ (33.51 mg, 45.80 μmol, 0.1 eq). The mixture was stirred under N₂ atmosphere at 130° C. for 3 h. The reaction mixture was concentrated to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 49%-79%, 10 min), followed by lyophilization to yield 3-(1-methylimidazol-4-yl)-N-(2,2,2-trifluoroethyl)-4-[[4-(trifluoromethyl)phenyl]methylamino]benzenesulfonamide (66.05 mg, 131.70 μmol, 28.7% yield, 98.1% purity) as a gray solid. ¹H NMR (400 MHZ, CDCl₃) δ ppm 9.39-9.28 (m, 1H), 7.87 (d, J=2.3 Hz, 1H), 7.59 (d, J=7.8 Hz, 2H), 7.53-7.45 (m, 4H), 7.30 (d, J=1.2 Hz, 1H), 6.53 (d, J=9.0 Hz, 1H), 4.75 (s, 1H), 4.60 (d, J=5.9 Hz, 2H), 3.78 (s, 3H), 3.59 (d, J=5.5 Hz, 2H); ES-LCMS m/z 493.1 [M+H]⁺.

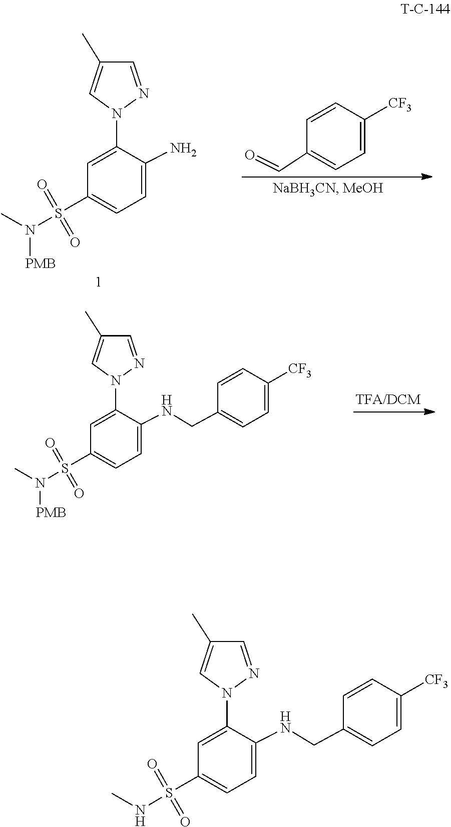

### Step 1: N-[(4-Methoxyphenyl)methyl]-N-methyl-3-(4-methylpyrazol-1-yl)-4-[[4-(trifluoromethyl)phenyl]methylamino]benzenesulfonamide

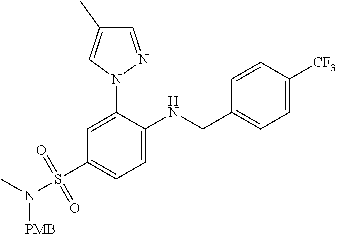

To a solution of 4-amino-N-[(4-methoxyphenyl)methyl]-N-methyl-3-(4-methylpyrazol-1-yl)benzenesulfonamide (50 mg, 116.44 μmol, 90% purity, 1 eq) in MeOH (1 mL) was added 4-(trifluoromethyl)benzaldehyde (22.30 mg, 128.08 μmol, 17.16 μL, 1.1 eq). The mixture was stirred at 25° C. for 2 h. NaBH₃CN (36.59 mg, 582.20 μmol, 5 eq) was added and the mixture was stirred at 25° C. for 12 h. The mixture was diluted with water (5 mL) and extracted with EtOAc (5 mL×3). The combined organic layers were dried over Na₂SO₄, filtered and concentrated under reduced pressure to yield a residue which was purified by preparative TLC (PE/EtOAc=2/1, R_{f}=0.60) to yield N-[(4-methoxyphenyl)methyl]-N-methyl-3-(4-methylpyrazol-1-yl)-4-[[4-(trifluoromethyl)phenyl]methylamino]benzenesulfonamide (12 mg, 19.83 μmol, 17.0% yield, 90.0% purity) as a white solid. ¹H NMR (400 MHZ, CDCl₃) δ ppm 7.65-7.54 (m, 6H), 7.48 (d, J=8.2 Hz, 2H), 7.22 (d, J=8.6 Hz, 2H), 6.86 (d, J=8.6 Hz, 2H), 6.68 (d, J=8.6 Hz, 1H), 4.56 (d, J=5.9 Hz, 2H), 4.05 (s, 2H), 3.80 (s, 3H), 2.55 (s, 3H), 2.24-2.15 (m, 3H); ES-LCMS m/z 545.2 [M+H]⁺.

### Step 2: N-Methyl-3-(4-methylpyrazol-1-yl)-4-[[4-(trifluoromethyl)phenyl]methylamino]benzenesulfonamide

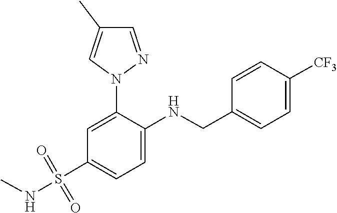 [SMILES: CNS(=O)(=O)c1ccc(NCc2ccc(C(F)(F)F)cc2)c(-n2cc(C)cn2)c1]

A solution of N-[(4-methoxyphenyl)methyl]-N-methyl-3-(4-methylpyrazol-1-yl)-4-[[4-(trifluoromethyl)phenyl]methylamino]benzenesulfonamide (12 mg, 19.83 μmol, 90% purity, 1 eq) in DCM (1 mL) and TFA (0.2 mL) was stirred at 25° C. for 12 h. The mixture was filtered and concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 45%-75%, 10 min), followed by lyophilization to yield N-methyl-3-(4-methylpyrazol-1-yl)-4-[[4-(trifluoromethyl)phenyl]methylamino]benzenesulfonamide (3.38 mg, 7.95 μmol, 40.1% yield, 99.8% purity) as a white solid. ¹H NMR (500 MHZ, CDCl₃) δ ppm 7.68 (d, J=2.1 Hz, 1H), 7.64 (s, 1H), 7.62-7.56 (m, 5H), 7.46 (d, J=8.1 Hz, 2H), 6.65 (d, J=8.9 Hz, 1H), 4.55 (d, J=5.8 Hz, 2H), 4.22 (d, J=4.9 Hz, 1H), 2.64 (d, J=5.0 Hz, 3H), 2.19 (s, 3H); ES-LCMS m/z 425.2 [M+H]⁺.

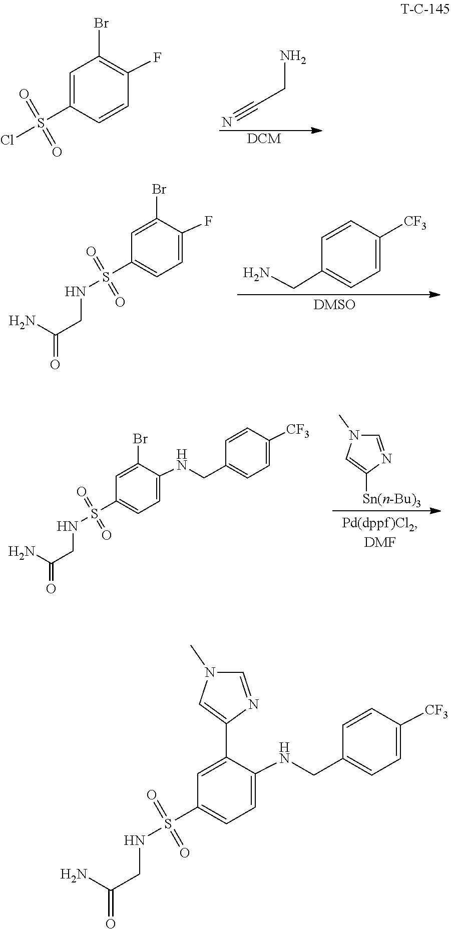

### Step 1: 2-[(3-Bromo-4-fluoro-phenyl)sulfonylamino]acetamide

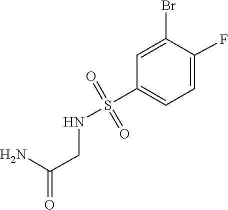 [SMILES: NC(=O)CNS(=O)(=O)c1ccc(F)c(Br)c1]

To a solution of 3-bromo-4-fluoro-benzenesulfonyl chloride (300 mg, 1.10 mmol, 1 eq) in THF (8 mL) was added DIEA (296.80 mg, 2.30 mmol, 400 μL, 2.09 eq) and 2-aminoacetamide (160 mg, 2.16 mmol, 1.97 eq) at 0° C. The mixture was stirred at 0° C. for 1 h and at 20° C. for 1 h. The reaction mixture was quenched with H₂O (30 mL) and extracted with EtOAc (30 mL×3). The combined organic layers dried over Na₂SO₄, filtered and concentrated under reduced pressure to yield 2-[(3-bromo-4-fluoro-phenyl)sulfonylamino]acetamide (330 mg, 1.03 mmol, 93.8% yield, 97.0%) as a white solid, which was used in the next step without further purification. ¹H NMR (400 MHZ, DMSO-d₆) δ ppm 8.10 (dd, J=2.2, 6.4 Hz, 1H), 7.97 (s, 1H), 7.84 (ddd, J=2.2, 4.6, 8.6 Hz, 1H), 7.59 (t, J=8.6 Hz, 1H), 7.32 (s, 1H), 7.09 (s, 1H), 3.45 (s, 2H); ES-LCMS m/z 311.0, 313.0 [M+H]⁺.

### Step 2: 2-[[3-Bromo-4-[[4-(trifluoromethyl)phenyl]methylamino]phenyl]sulfonylamino]acetamide

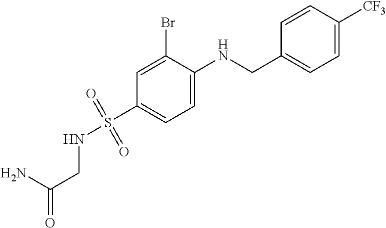 [SMILES: NC(=O)CNS(=O)(=O)c1ccc(NCc2ccc(C(F)(F)F)cc2)c(Br)c1]

To a solution of 2-[(3-bromo-4-fluoro-phenyl)sulfonylamino]acetamide (200 mg, 623.54 μmol, 97%, 1 eq) in DMSO (2 mL) was added [4-(trifluoromethyl)phenyl]methanamine (210 mg, 1.20 mmol, 170.73 μL, 1.92 eq). The mixture was stirred under microwave at 140° C. for 1 h. TLC (PE/EtOAc=1/1, R_{f}=0.20) showed the starting material was consumed completely. The reaction mixture was quenched with H₂O (30 mL) and extracted with EtOAc (30 mL×3). The combined organic layers dried over Na₂SO₄, filtered and concentrated under reduced pressure to yield a residue which was purified by flash silica gel chromatography (from PE/EtOAc=100/1 to 1/1, TLC: PE/EtOAc=1/1, R_{f}=0.20) to yield 2-[[3-bromo-4-[[4-(trifluoromethyl)phenyl]methylamino]phenyl]sulfonylamino]acetamide (200 mg, 386.04 μmol, 61.9% yield, 90.0%) as a yellow solid. ¹H NMR (400 MHZ, DMSO-d₆) δ ppm 7.77 (d, J=2.0 Hz, 1H), 7.66 (d, J=8.1 Hz, 2H), 7.50 (d, J=8.1 Hz, 2H), 7.42 (dd, J=2.0, 8.6 Hz, 1H), 7.21 (s, 1H), 7.05 (s, 1H), 6.87 (t, J=6.1 Hz, 1H), 6.54 (d, J=8.8 Hz, 1H), 4.55 (d, J=6.1 Hz, 2H), 3.24 (s, 2H); ES-LCMS m/z 466.0, 468.0 [M+H]⁺.

### Step 3: 2-[[3-(1-Methylimidazol-4-yl)-4-[[4-(trifluoromethyl)phenyl]methylamino]phenyl]sulfonylamino]acetamide

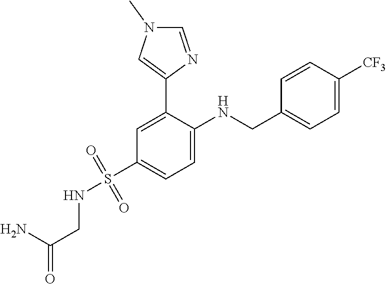 [SMILES: Cn1cnc(-c2cc(S(=O)(=O)NCC(N)=O)ccc2NCc2ccc(C(F)(F)F)cc2)c1]

To a solution of 2-[[3-bromo-4-[[4-(trifluoromethyl)phenyl]methylamino]phenyl]sulfonylamino]acetamide (200 mg, 386.04 μmol, 90%, 1 eq) in DMF (2 mL) was added Pd(dppf)Cl₂ (30 mg, 41.00 μmol, 0.1 eq) and tributyl-(1-methylimidazol-4-yl)stannane (210 mg, 565.81 μmol, 1.47 eq). The mixture was stirred under N₂ atmosphere at 130° C. for 2 h. The reaction mixture was quenched with H₂O (40 mL) and extracted with EtOAc (50 mL×3). The combined organic layers washed with brine (50 mL), dried over Na₂SO₄, filtered and concentrated under reduced pressure to yield a residue which was purified by preparative HPLC (column: Welch Xtimate C18 150×25 mm×5 μm; mobile phase: [water (10 mM NH₄HCO₃)-ACN]; B %: 34%-64%, 10 min) to yield 2-[[3-(1-methylimidazol-4-yl)-4-[[4-(trifluoromethyl)phenyl]methylamino]phenyl]sulfonylamino]acetamide (60 mg, 126.30 μmol, 32.7% yield, 98.4%) as a white solid. ¹H NMR (400 MHZ, DMSO-d₆) δ ppm 9.33 (t, J=5.5 Hz, 1H), 7.85 (d, J=2.2 Hz, 1H), 7.81 (s, 1H), 7.73-7.67 (m, 3H), 7.56 (d, J=8.1 Hz, 2H), 7.39-7.29 (m, 2H), 7.26-7.20 (m, 1H), 7.11 (s, 1H), 6.61 (d, J=8.8 Hz, 1H), 4.64 (d, J=5.6 Hz, 2H), 3.75 (s, 3H), 3.27 (s, 2H); ES-LCMS m/z 468.1 [M+H]⁺.

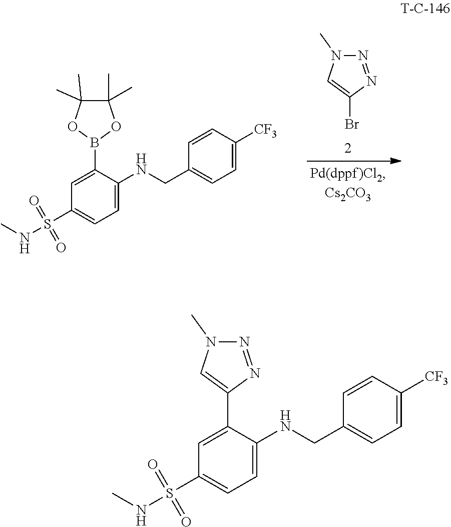

### Step 1. N-Methyl-3-(1-methyltriazol-4-yl)-4-[[4-(trifluoromethyl)phenyl]methylamino]benzenesulfonamide

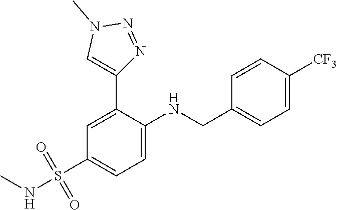

To a solution of N-methyl-3-(4,4,5,5-tetramethyl-1,3,2-dioxaborolan-2-yl)-4-[[4-(trifluoromethyl)phenyl]methylamino]benzenesulfonamide (300 mg, 510.30 μmol, 80% purity, 1 eq) and 4-bromo-1-methyl-triazole (123.99 mg, 765.45 μmol, 1.5 eq) in 1,4-dioxane (5 mL) and H₂O (2 mL) was added Pd(dppf)Cl₂ (37.34 mg, 51.03 μmol, 0.1 eq) and Cs₂CO₃ (332.53 mg, 1.02 mmol, 2 eq). The mixture was stirred at 100° C. for 12 h. The reaction mixture was quenched by addition of water (50 mL) and extracted with EtOAc (30 mL×3). The combined organic layers were washed with brine (30 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 42%-72%, 10 min), followed by lyophilization to yield N-methyl-3-(1-methyltriazol-4-yl)-4-[[4-(trifluoromethyl)phenyl]methylamino]benzenesulfonamide (80.15 mg, 188.40 μmol, 36.9% yield, 100.0% purity) as a white solid. ¹H NMR (400 MHZ, CDCl₃) δ ppm 8.92 (t, J=5.6 Hz, 1H), 7.98 (s, 1H), 7.91 (d, J=2.2 Hz, 1H), 7.61 (d, J=8.3 Hz, 2H), 7.56 (dd, J=2.0, 8.8 Hz, 1H), 7.49 (d, J=8.1 Hz, 2H), 6.62 (d, J=8.8 Hz, 1H), 4.65 (d, J=5.9 Hz, 2H), 4.27 (d, J=5.4 Hz, 1H), 4.21 (s, 3H), 2.63 (d, J=5.4 Hz, 3H); ES-LCMS m/z 426.2 [M+H]⁺.

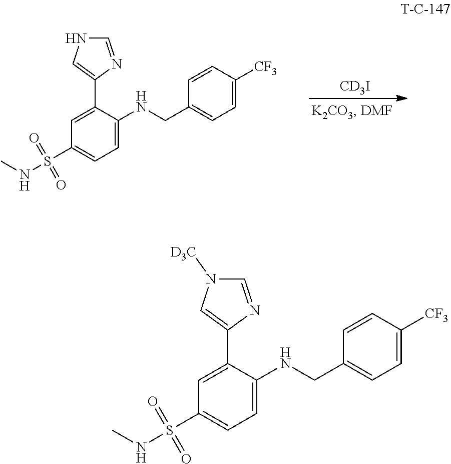 [SMILES: CNS(=O)(=O)c1ccc(NCc2ccc(C(F)(F)F)cc2)c(-c2c[nH]cn2)c1 ; "CD3I" ; [2H]C([2H])([2H])n1cnc(-c2cc(S(=O)(=O)NC)ccc2NCc2ccc(C(F)(F)F)cc2)c1]

### Step 1: N-Methyl-3-[1-(trideuteriomethyl)imidazol-4-yl]-4-[[4-(trifluoromethyl)phenyl]methylamino]benzenesulfonamide

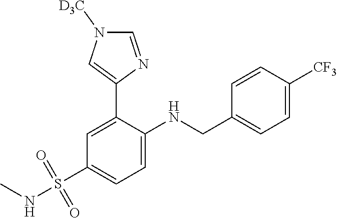 [SMILES: [2H]C([2H])([2H])n1cnc(-c2cc(S(=O)(=O)NC)ccc2NCc2ccc(C(F)(F)F)cc2)c1]

To a stirred solution of 3-(1H-imidazol-4-yl)-N-methyl-4-[[4-(trifluoromethyl)phenyl]methylamino]benzenesulfonamide (500 mg, 1.13 mmol, 93.0% purity, 1 eq) in DMF (20 mL) was added trideuterio(iodo)methane (361.32 mg, 2.49 mmol, 155.07 μL, 2.2 eq) and K₂CO₃ (187.91 mg, 1.36 mmol, 1.2 eq). The reaction mixture was stirred under N₂ atmosphere at 25° C. for 3 h. The reaction mixture was diluted with H₂O (20 mL) and extracted with EtOAc (50 mL×3). The combined organic layers were washed with brine (20 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 50%-80%, 10 min) to yield N-methyl-3-[1-(trideuteriomethyl)imidazol-4-yl]-4-[[4-(trifluoromethyl)phenyl]methylamino]benzenesulfonamide (240 mg, 551.77 μmol, 48.7% yield, 98.3% purity) as a white solid. ¹H NMR (400 MHZ, CDCl₃) δ ppm 9.24 (s, 1H), 7.88 (d, J=2.0 Hz, 1H), 7.59 (d, J=8.1 Hz, 2H), 7.49 (d, J=6.8 Hz, 4H), 7.32 (d, J=1.0 Hz, 1H), 6.54 (d, J=8.8 Hz, 1H), 4.60 (d, J=5.6 Hz, 2H), 4.21 (d, J=5.4 Hz, 1H), 2.62 (d, J=5.6 Hz, 3H); ES-LCMS m/z 428.2 [M+H]⁺.

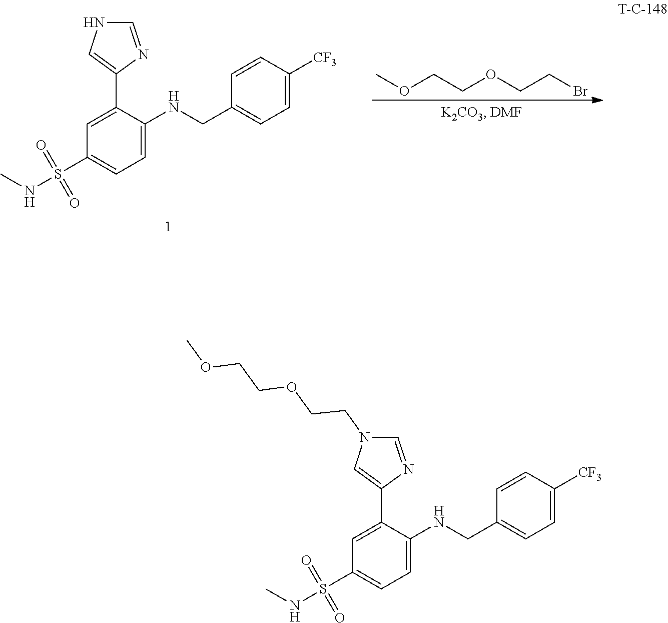

### Step 1: N-[(4-Methoxyphenyl)methyl]-N-methyl-4-[[5-(trifluoromethyl)-2-pyridyl]amino]-3-vinyl-benzenesulfonamide

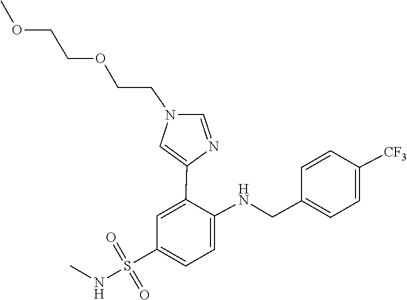 [SMILES: CNS(=O)(=O)c1ccc(NCc2ccc(C(F)(F)F)cc2)c(-c2cn(CCOCCOC)cn2)c1]

To a solution of 3-(1H-imidazol-4-yl)-N-methyl-4-[[4-(trifluoromethyl)phenyl]methylamino]benzenesulfonamide (50 mg, 103.55 μmol, 85%, 1 eq) in DMF (5 mL) was added K₂CO₃ (28.62 mg, 207.11 μmol, 2 eq) and 1-(2-bromoethoxy)-2-methoxy-ethane (37.91 mg, 207.11 μmol, 2 eq). The mixture was stirred at 25° C. for 12 h. The reaction mixture was quenched by addition of water (50 mL) and extracted with EtOAc (30 mL×3). The combined organic layers were washed with brine (30 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 42%-72%, 10 min), followed by lyophilization to yield 3-[1-[2-(2-methoxyethoxy)ethyl]imidazol-4-yl]-N-methyl-4-[[4-(trifluoromethyl)phenyl]methylamino]benzenesulfonamide (24.95 mg, 48.34 μmol, 46.6% yield, 99.3% purity) as a yellow solid. ¹H NMR (400 MHZ, CDCl₃) δ ppm 9.30 (t, J=5.6 Hz, 1H), 7.90 (d, J=2.2 Hz, 1H), 7.59 (d, J=7.3 Hz, 3H), 7.52-7.47 (m, 3H), 7.43 (d, J=1.0 Hz, 1H), 6.54 (d, J=8.8 Hz, 1H), 4.60 (d, J=5.9 Hz, 2H), 4.22-4.16 (m, 3H), 3.82 (t, J=5.0 Hz, 2H), 3.66-3.61 (m, 2H), 3.57-3.52 (m, 2H), 3.40 (s, 3H), 2.62 (d, J=5.6 Hz, 3H); ES-LCMS m/z 513.2 [M+H]⁺.

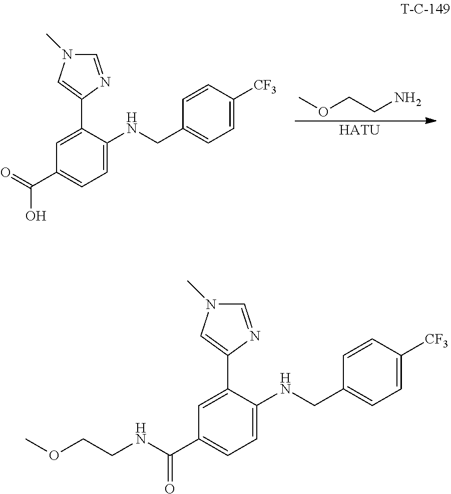

### Step 1: N-(2-Methoxyethyl)-3-(1-methyl-1H-imidazol-4-yl)-4-((4-(trifluoromethyl)benzyl)amino)benzamide

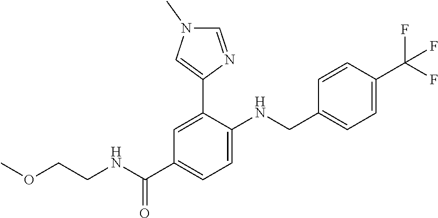 [SMILES: COCCNC(=O)c1ccc(NCc2ccc(C(F)(F)F)cc2)c(-c2cn(C)cn2)c1]

To a solution of 3-(1-methylimidazol-4-yl)-4-[[4-(trifluoromethyl)phenyl]methylamino]benzoic acid (51.02 mg, 133.21 μmol, 98%, 1 eq) in DMF (2 mL) was added HATU (60.78 mg, 159.85 μmol, 1.2 eq), 2-methoxyethanamine (16.01 mg, 213.14 μmol, 18.53 μL, 1.6 eq) and TEA (40.44 mg, 399.63 μmol, 55.62 μL, 3 eq). The reaction mixture was concentrated to yield a residue which was purified by preparative HPLC (basic) to yield N-(2-methoxyethyl)-3-(1-methyl-1H-imidazol-4-yl)-4-((4-(trifluoromethyl)benzyl)amino)benzamide (26.85 mg, 62.09 μmol, 46.6% yield, 100.0% purity) as a green solid. ¹H NMR (400 MHZ, CDCl₃) δ ppm 8.98 (br s, 1H), 7.98 (d, J=2.3 Hz, 1H), 7.60-7.55 (m, 2H), 7.49 (d, J=9.4 Hz, 3H), 7.40 (dd, J=2.3, 8.6 Hz, 1H), 7.32 (d, J=1.6 Hz, 1H), 6.48 (d, J=8.6 Hz, 1H), 6.39 (br s, 1H), 4.60 (d, J=5.9 Hz, 2H), 3.78 (s, 3H), 3.63 (q, J=5.2 Hz, 2H), 3.58-3.50 (m, 2H), 3.38 (s, 3H); ES-LCMS m/z 433.2 [M+H]⁺.

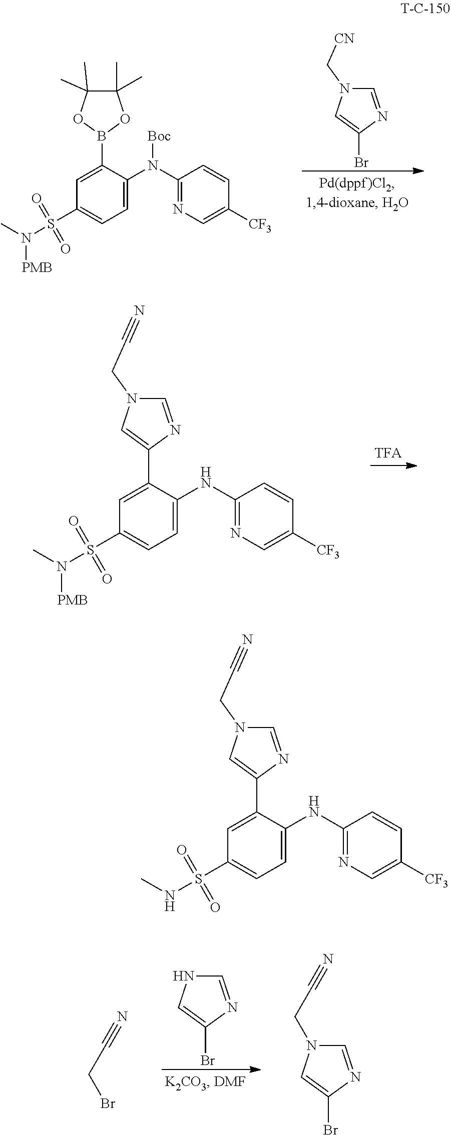

### Step 1: 3-[1-(Cyanomethyl)imidazol-4-yl]-N-[(4-methoxyphenyl)methyl]-N-methyl-4-[[5-(trifluoromethyl)-2-pyridyl]amino]benzenesulfonamide

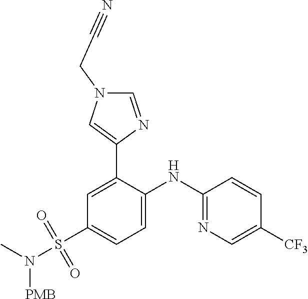

A mixture of 2-(4-bromoimidazol-1-yl)acetonitrile (150 mg, 806.41 μmol, 100% purity, 1 eq), tert-butyl N-[4-[(4-methoxyphenyl)methyl-methyl-sulfamoyl]-2-(4,4,5,5-tetramethyl-1,3,2-dioxaborolan-2-yl)phenyl]-N-[5-(trifluoromethyl)-2-pyridyl]carbamate (500 mg, 652.36 μmol, 88.4% purity, 8.09e-1 eq), Pd(PPh₃)₄ (100 mg, 86.54 μmol, 1.07e-1 eq) and Cs₂CO₃ (800 mg, 2.46 mmol, 3.04 eq) in 1,4-dioxane (3 mL) and H₂O (1 mL) was stirred under N₂ atmosphere at 100° C. for 2 h. The reaction mixture was diluted with H₂O (20 mL) and extracted with EtOAc (20 mL×3). The organic layer was dried over Na₂SO₄, filtered and concentrated under reduced pressure to yield a residue which was purified by flash silica gel chromatography (from PE/EtOAc=100/1 to 0/1, TLC: PE/EtOAc=1/1, R_{f}=0.16, 0.05) to yield 3-[1-(cyanomethyl)imidazol-4-yl]-N-[(4-methoxyphenyl)methyl]-N-methyl-4-[[5-(trifluoromethyl)-2-pyridyl]amino]benzenesulfonamide (30 mg, 35.41 μmol, 4.4% yield, 65.7% purity) as a colorless gum. ES-LCMS m/z 557.2 [M+H]⁺.

### Step 2: 3-[1-(Cyanomethyl)imidazol-4-yl]-N-methyl-4-[[5-(trifluoromethyl)-2-pyridyl]amino]benzenesulfonamide

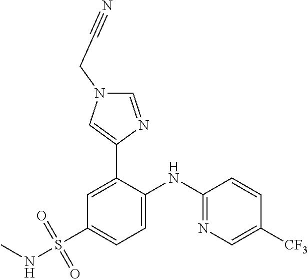 [SMILES: CNS(=O)(=O)c1ccc(Nc2ccc(C(F)(F)F)cn2)c(-c2cn(CC#N)cn2)c1]

To a solution of 3-[1-(cyanomethyl)imidazol-4-yl]-N-[(4-methoxyphenyl)methyl]-N-methyl-4-[[5-(trifluoromethyl)-2-pyridyl]amino]benzenesulfonamide (30 mg, 53.90 μmol, 1 eq) in DCM (2 mL) was added TFA (770.00 mg, 6.75 mmol, 0.5 mL, 125.28 eq). The mixture was stirred at 25° C. for 1 h. The reaction mixture was concentrated under reduced pressure to yield a residue which was purified by preparative HPLC (column: Welch Xtimate C18 150*25 mm*5 μm; mobile phase: [water (10 mM NH₄HCO₃)-ACN]; B %: 41%-71%, 10 min) and lyophilized to yield 3-[1-(cyanomethyl)imidazol-4-yl]-N-methyl-4-[[5-(trifluoromethyl)-2-pyridyl]amino]benzenesulfonamide (1.76 mg, 4.03 μmol, 7.5% yield, 100.0% purity) as a white solid. ¹H NMR (500 MHZ, CD₃OD) δ ppm 8.81 (d, J=8.9 Hz, 1H), 8.53 (s, 1H), 8.11 (d, J=2.1 Hz, 1H), 7.99 (s, 1H), 7.87 (dd, J=2.4, 8.9 Hz, 1H), 7.83 (s, 1H), 7.72 (dd, J=2.2, 8.8 Hz, 1H), 7.06 (d, J=8.9 Hz, 1H), 5.37 (s, 2H), 2.58 (s, 3H); ES-LCMS m/z 437.2 [M+H]⁺.

### Step 3: 2-(4-Bromoimidazol-1-yl)acetonitrile

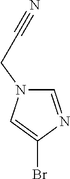

To a solution of 2-bromoacetonitrile (2.45 g, 20.41 mmol, 1.36 mL, 1.2 eq) and 4-bromo-1H-imidazole (2.5 g, 17.01 mmol, 1 eq) in DMF (12 mL) was added K₂CO₃ (4.70 g, 34.02 mmol, 2 eq). The mixture was stirred at 50° C. for 3 h. The reaction mixture was quenched with H₂O (100 mL) and extracted with EtOAc (100 mL×3). The combined organic layers was washed with brine (100 mL), dried over Na₂SO₄, filtered and concentrated under reduced pressure to yield a residue which was purified by preparative HPLC (column: Welch Xtimate C18 150×25 mm×5 μm; mobile phase: [water (10 mM NH₄HCO₃)-ACN]; B %: 0%-40%, 10 min) and lyophilized to yield 2-(4-bromoimidazol-1-yl)acetonitrile (1.5 g, 8.06 mmol, 47.4% yield, 90.0%) as a brown solid. ¹H NMR (400 MHZ, DMSO-d₆) δ ppm 7.78 (d, J=1.2 Hz, 1H), 7.51 (d, J=1.5 Hz, 1H), 5.33 (s, 2H); ES-LCMS m/z 186.1, 188.1 [M+H]⁺.

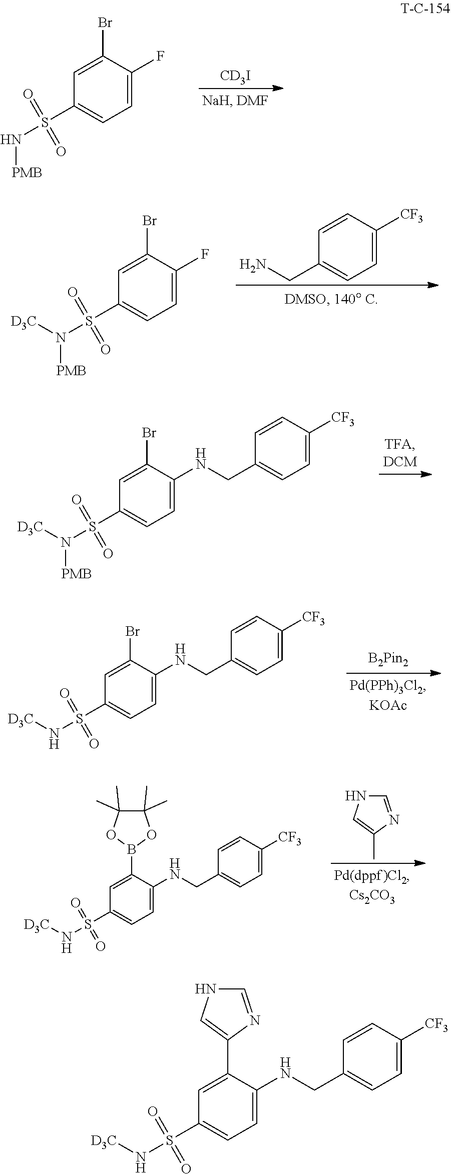

### Step 1: 3-Bromo-4-fluoro-N-[(4-methoxyphenyl)methyl]-N-(trideuteriomethyl)benzenesulfonamide

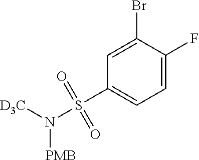

To a solution of 3-bromo-4-fluoro-N-[(4-methoxyphenyl)methyl]benzenesulfonamide (3.2 g, 8.12 mmol, 95%, 1 eq) in THF (50 mL) was added NaH (649.88 mg, 16.25 mmol, 60%, 2 eq) at 0° C. The mixture was stirred at 0° C. for 30 min. Trideuterio(iodo)methane (2.31 g, 16.25 mmol, 989.74 μL, 2 eq) was added to the mixture dropwise with stirring at 0° C. The mixture was stirred at 0° C. for 1 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield 3-bromo-4-fluoro-N-[(4-methoxyphenyl)methyl]-N-(trideuteriomethyl)benzenesulfonamide (3 g, 7.67 mmol, 94.3% yield, 100.0% purity) as a white solid, which was used in the next step without further purification. ¹H NMR (400 MHZ, CDCl₃) δ ppm 8.04 (dd, J=2.1, 6.2 Hz, 1H), 7.78 (m, 1H), 7.32-7.27 (m, 1H), 7.22 (d, J=8.3 Hz, 2H), 6.88 (d, J=8.6 Hz, 2H), 4.12 (s, 2H), 3.82 (s, 3H); ES-LCMS: no desired m/z was found.

### Step 2: 3-Bromo-N-[(4-methoxyphenyl)methyl]-N-(trideuteriomethyl)-4-[[4-(trifluoromethyl)phenyl]methylamino]benzenesulfonamide

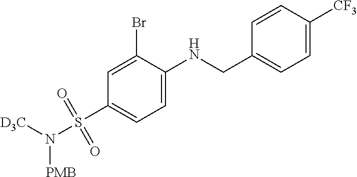

To a solution of [4-(trifluoromethyl)phenyl]methanamine (2.69 g, 15.33 mmol, 2.18 mL, 2 eq) in DMSO (30 mL) was added 3-bromo-4-fluoro-N-[(4-methoxyphenyl)methyl]-N-(trideuteriomethyl)benzenesulfonamide (3 g, 7.67 mmol, 1 eq). The mixture was stirred at 140° C. for 12 h. The reaction mixture was quenched by addition of water (80 mL) and extracted with EtOAc (60 mL×3). The combined organic layers were washed with brine (10 mL), dried over Na₂SO₄, filtered and concentrated to yield a residue which was purified by flash silica gel chromatography (from PE/EtOAc=1/0 to 5/1, TLC: PE/EtOAc=5/1, R_{f}=0.58) to yield 3-bromo-N-[(4-methoxyphenyl)methyl]-N-(trideuteriomethyl)-4-[[4-(trifluoromethyl)phenyl]methylamino]benzenesulfonamide (3.2 g, 5.27 mmol, 68.7% yield, 90.0% purity) as yellow oil. ¹H NMR (400 MHZ, CDCl₃) δ ppm 7.93 (d, J=1.2 Hz, 1H), 7.66 (d, J=8.1 Hz, 2H), 7.58 (d, J=9.8 Hz, 1H), 7.48 (d, J=8.1 Hz, 2H), 7.22 (d, J=8.3 Hz, 2H), 6.86 (d, J=8.1 Hz, 2H), 6.57 (d, J=8.6 Hz, 1H), 5.36 (t, J=5.5 Hz, 1H), 4.57 (d, J=5.6 Hz, 2H), 4.05 (s, 2H), 3.81 (s, 3H); ES-LCMS m/z 546.1, 548.1 [M+H]⁺.

### Step 3: 3-Bromo-N-(trideuteriomethyl)-4-[[4-(trifluoromethyl)phenyl]methylamino]benzenesulfonamide

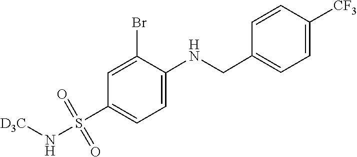 [SMILES: [2H]C([2H])([2H])NS(=O)(=O)c1ccc(NCc2ccc(C(F)(F)F)cc2)c(Br)c1]

To a solution of 3-bromo-N-[(4-methoxyphenyl)methyl]-N-(trideuteriomethyl)-4-[[4-(trifluoromethyl)phenyl]methylamino]benzenesulfonamide (3.1 g, 5.11 mmol, 90%, 1 eq) in DCM (40 mL) was added TFA (11.09 g, 97.24 mmol, 7.20 mL, 19.04 eq). The mixture was stirred at 25° C. for 12 h. The reaction mixture was quenched by addition of water (60 mL) and extracted with EtOAc (50 mL×3). The combined organic layers were washed with brine (20 mL), dried over Na₂SO₄, filtered and concentrated under reduced pressure to yield 3-bromo-N-(trideuteriomethyl)-4-[[4-(trifluoromethyl)phenyl]methylamino]benzenesulfonamide (2.2 g, 4.64 mmol, 90.9% yield, 90.0% purity) as a yellow solid, which was used in the next step without further purification. ¹H NMR (400 MHZ, CDCl₃) δ ppm 7.97 (d, J=2.2 Hz, 1H), 7.64 (d, J=8.1 Hz, 2H), 7.60 (dd, J=2.1, 8.7 Hz, 1H), 7.46 (d, J=8.1 Hz, 2H), 6.54 (d, J=8.8 Hz, 1H), 5.35 (s, 1H), 4.57 (d, J=4.6 Hz, 2H), 4.21-4.15 (m, 1H); ES-LCMS m/z 426.1, 428.1 [M+H]⁺.

### Step 4: 3-(4,4,5,5-Tetramethyl-1,3,2-dioxaborolan-2-yl)-N-(trideuteriomethyl)-4-[[4-(trifluoromethyl)phenyl]methylamino]benzenesulfonamide

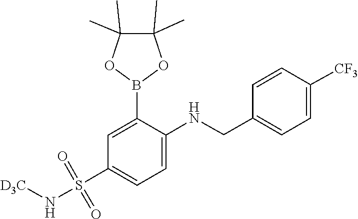 [SMILES: [2H]C([2H])([2H])NS(=O)(=O)c1ccc(NCc2ccc(C(F)(F)F)cc2)c(B2OC(C)(C)C(C)(C)O2)c1]

To a solution of 4,4,5,5-tetramethyl-2-(4,4,5,5-tetramethyl-1,3,2-dioxaborolan-2-yl)-1,3,2-dioxaborolane (3.22 g, 12.67 mmol, 3 eq) and 3-bromo-N-(trideuteriomethyl)-4-[[4-(trifluoromethyl)phenyl]methylamino]benzenesulfonamide (2 g, 4.22 mmol, 90%, 1 eq) in 1,4-dioxane (10 mL) was added Pd(dppf)Cl₂ (308.98 mg, 422.27 μmol, 0.1 eq) and KOAc (828.83 mg, 8.45 mmol, 2 eq). The mixture was stirred under microwave (1 bar) at 75° C. for 1 h. The reaction mixture was quenched by addition of water (60 mL) and extracted with EtOAc (50 mL×3). The combined organic layers were washed with brine (10 mL), dried over Na₂SO₄, filtered and concentrated to yield a residue which was purified by flash silica gel chromatography (from PE/EtOAc=1/0 to 3/1, TLC: PE/EtOAc=3/1, R_{f}=0.64) to yield 3-(4,4,5,5-tetramethyl-1,3,2-dioxaborolan-2-yl)-N-(trideuteriomethyl)-4-[[4-(trifluoromethyl)phenyl]methylamino]benzenesulfonamide (1.5 g, 2.85 mmol, 67.5% yield, 90.0% purity) as a white solid. ¹H NMR (400 MHZ, CDCl₃) δ ppm 8.14 (d, J=2.2 Hz, 1H), 7.68 (dd, J=2.4, 8.8 Hz, 1H), 7.62 (d, J=8.1 Hz, 2H), 7.46 (d, J=8.1 Hz, 2H), 6.91 (t, J=5.5 Hz, 1H), 6.44 (d, J=8.8 Hz, 1H), 4.53 (d, J=5.6 Hz, 2H), 1.36 (s, 12H); ES-LCMS m/z 474.1[M+H]⁺.

### Step 5: 3-(1H-Imidazol-4-yl)-N-(trideuteriomethyl)-4-[[4-(trifluoromethyl)phenyl]methylamino]benzenesulfonamide

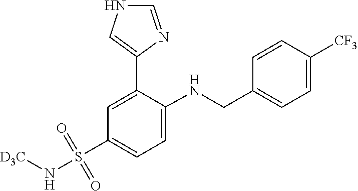 [SMILES: [2H]C([2H])([2H])NS(=O)(=O)c1ccc(NCc2ccc(C(F)(F)F)cc2)c(-c2c[nH]cn2)c1]

To a solution of 4-iodo-1H-imidazole (553.24 mg, 2.85 mmol, 1 eq), 3-(4,4,5,5-tetramethyl-1,3,2-dioxaborolan-2-yl)-N-(trideuteriomethyl)-4-[[4-(trifluoromethyl)phenyl]methylamino]benzenesulfonamide (1.5 g, 2.85 mmol, 90%, 1 eq) in 1,4-dioxane (20 mL) and H₂O (4 mL) was added Pd(dppf)Cl₂ (208.69 mg, 285.21 μmol, 0.1 eq) and Cs₂CO₃ (1.86 g, 5.70 mmol, 2 eq). The mixture was stirred under N₂ atmosphere at 80° C. for 12 h. The reaction mixture was quenched by addition of water (50 mL) and extracted with EtOAc (30 mL×3). The combined organic layers were washed with brine (10 mL), dried over Na₂SO₄, filtered and concentrated to yield a residue which was purified by flash silica gel chromatography (from PE/EtOAc=1/0 to 1/1, TLC: PE/EtOAc=1/1, R_{f}=0.36) and by preparative HPLC (column: Agela DuraShell C18 150*25 mm*5 μm; mobile phase: [water (0.05% NH₃·H₂O+10 mM NH₄HCO₃)-ACN]; B %: 40%-70%, 10 min), followed by lyophilization to yield 3-(1H-imidazol-4-yl)-N-(trideuteriomethyl)-4-[[4-(trifluoromethyl)phenyl]methylamino]benzenesulfonamide (500 mg, 1.21 mmol, 42.4% yield, 100.0% purity) as a white solid. ¹H NMR (400 MHZ, CDCl₃) δ ppm 9.39 (s, 1H), 9.25 (s, 1H), 7.93 (d, J=1.7 Hz, 1H), 7.74 (s, 1H), 7.60 (d, J=8.3 Hz, 2H), 7.54-7.48 (m, 3H), 7.47 (s, 1H), 6.57 (d, J=8.6 Hz, 1H), 4.62 (d, J=5.6 Hz, 2H), 4.13 (s, 1H); ES-LCMS m/z 413.9 [M+H]⁺.

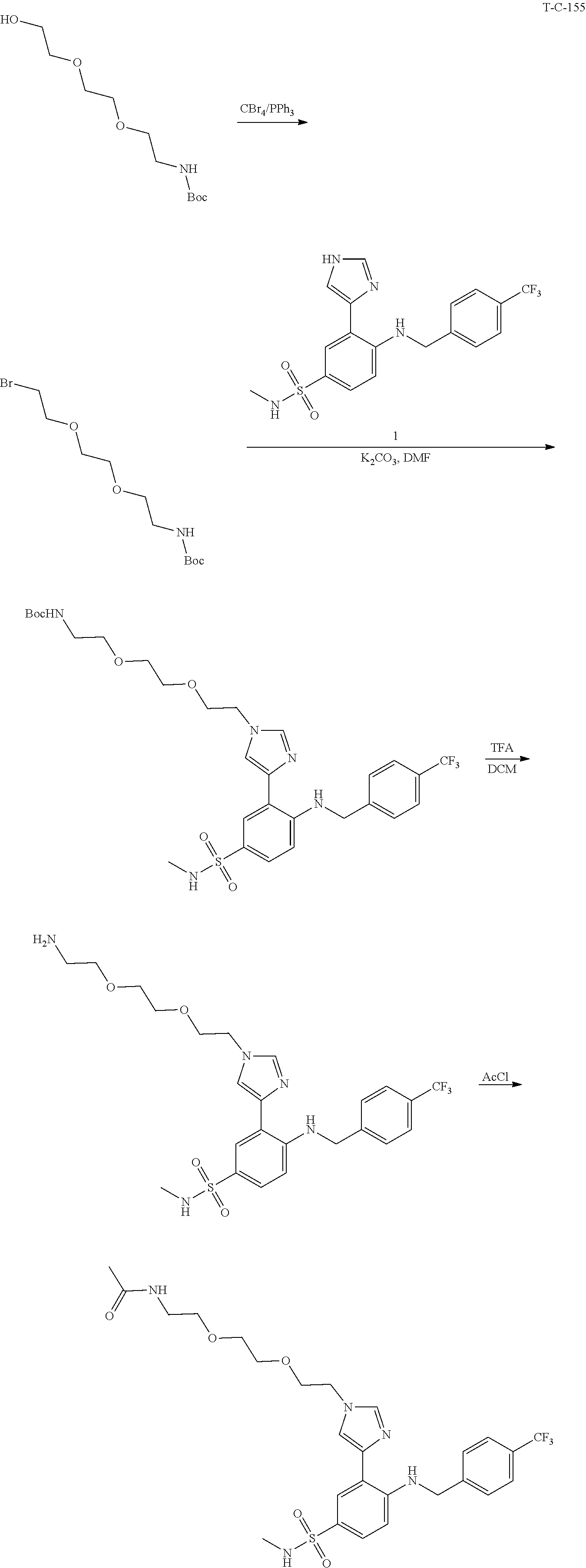

### Step 1: tert-Butyl N-[2-[2-(2-bromoethoxy)ethoxy]ethyl]carbamate

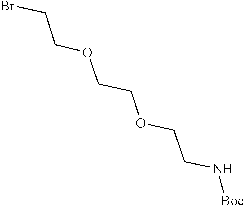 [SMILES: CC(C)(C)OC(=O)NCCOCCOCCBr]

To a solution of tert-butyl N-[2-[2-(2-hydroxyethoxy)ethoxy]ethyl]carbamate (300 mg, 1.20 mmol, 1 eq) and PPh₃ (631.24 mg, 2.41 mmol, 2 eq) in THF (5 mL) was added CBr₄ (798.13 mg, 2.41 mmol, 2 eq) slowly. The mixture was stirred at 25° C. for 3 h. The reaction mixture was diluted with water (20 mL) and extracted with EtOAc (30 mL×3). The combined organic phases were washed with brine (20 mL), dried over anhydrous Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.37) to yield tert-butyl N-[2-[2-(2-bromoethoxy)ethoxy]ethyl]carbamate (280 mg, 654.71 μmol, 54.4% yield, 73.0% purity) as yellow oil. ¹H NMR (400 MHZ, CDCl₃) δ ppm 5.03 (s, 1H), 3.81 (t, J=6.3 Hz, 2H), 3.69-3.60 (m, 4H), 3.55 (t, J=5.1 Hz, 2H), 3.48 (t, J=6.3 Hz, 2H), 3.36-3.27 (m, 2H), 1.43 (s, 9H).

### Step 2: tert-Butyl N-[2-[2-[2-[4-[5-(methylsulfamoyl)-2-[[4-(trifluoromethyl)phenyl]methylamino]phenyl]imidazol-1-yl]ethoxy]ethoxy]ethyl]carbamate

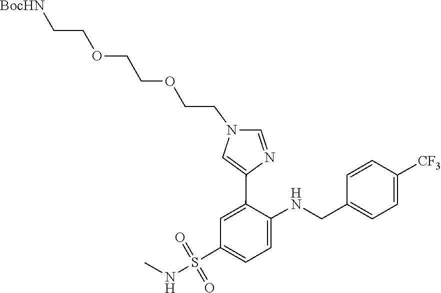 [SMILES: CNS(=O)(=O)c1ccc(NCc2ccc(C(F)(F)F)cc2)c(-c2cn(CCOCCOCCNC(=O)OC(C)(C)C)cn2)c1]

To a solution of 3-(1H-imidazol-4-yl)-N-methyl-4-[[4-(trifluoromethyl)phenyl]methylamino]benzenesulfonamide (250 mg, 527.52 μmol, 86.6% purity, 1 eq) in DMF (3 mL) was added K₂CO₃ (145.81 mg, 1.06 mmol, 2 eq) and tert-butyl N-[2-[2-(2-bromoethoxy)ethoxy]ethyl]carbamate (280 mg, 654.71 μmol, 73% purity, 1.24 eq). The mixture was stirred at 25° C. for 6 h. The reaction mixture was diluted with water (20 mL) and extracted with EtOAc (30 mL×3). The combined organic phases were washed with brine (20 mL), dried over anhydrous Na₂SO₄, filtered and concentrated under reduced pressure to yield a residue which was purified by flash silica gel chromatography (from PE/EtOAc=1/0 to 1/1, TLC: PE/EtOAc=1/1, R_{f}=0.15) to yield tert-butyl N-[2-[2-[2-[4-[5-(methylsulfamoyl)-2-[[4-(trifluoromethyl)phenyl]methylamino]phenyl]imidazol-1-yl]ethoxy]ethoxy]ethyl]carbamate (290 mg, 361.54 μmol, 68.5% yield, 80.0% purity) as a yellow gum. ¹H NMR (500 MHZ, CDCl₃) δ ppm 9.31 (t, J=5.7 Hz, 1H), 7.95-7.87 (m, 1H), 7.58 (d, J=6.4 Hz, 3H), 7.47 (d, J=10.7 Hz, 2H), 6.52 (d, J=8.9 Hz, 1H), 5.08 (s, 1H), 4.59 (d, J=5.8 Hz, 2H), 4.17 (t, J=4.9 Hz, 2H), 4.11-4.07 (m, 1H), 3.79 (t, J=5.0 Hz, 2H), 3.60 (s, 4H), 3.56-3.52 (m, 2H), 3.32 (d, J=4.6 Hz, 2H), 2.60 (d, J=5.3 Hz, 3H), 1.39 (s, 9H); ES-LCMS m/z 642.2 [M+H]⁺.

### Step 3: 3-[1-[2-[2-(2-Aminoethoxy)ethoxy]ethyl]imidazol-4-yl]-N-methyl-4-[[4-(trifluoromethyl)phenyl]methylamino]benzenesulfonamide

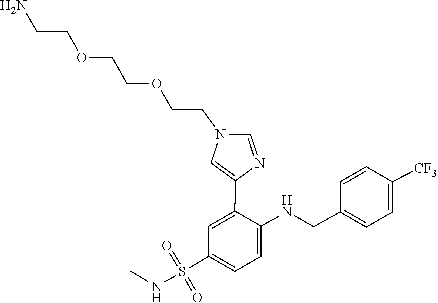 [SMILES: CNS(=O)(=O)c1ccc(NCc2ccc(C(F)(F)F)cc2)c(-c2cn(CCOCCOCCN)cn2)c1]

To a solution of tert-butyl N-[2-[2-[2-[4-[5-(methylsulfamoyl)-2-[[4-(trifluoromethyl)phenyl]methylamino]phenyl]imidazol-1-yl]ethoxy]ethoxy]ethyl]carbamate (290 mg, 361.54 μmol, 80% purity, 1 eq) in DCM (6 mL) was added TFA (1 mL). The mixture was stirred at 25° C. for 1 h. The solvent was removed to yield 3-[1-[2-[2-(2-aminoethoxy)ethoxy]ethyl]imidazol-4-yl]-N-methyl-4-[[4-(trifluoromethyl)phenyl]methylamino]benzenesulfonamide (170 mg, 142.62 μmol, 39.5% yield, 55.0% purity, TFA) as a green gum. ¹H NMR (500 MHz, CDCl₃) δ ppm 9.25 (t, J=5.8 Hz, 1H), 7.91 (d, J=2.1 Hz, 1H), 7.81-7.75 (m, 1H), 7.59 (s, 2H), 7.56-7.53 (m, 2H), 7.49-7.45 (m, 3H), 6.50 (d, J=8.9 Hz, 1H), 4.58 (d, J=5.5 Hz, 2H), 4.14-4.11 (m, 2H), 4.10-4.08 (m, 2H), 3.81-3.78 (m, 2H), 3.61-3.55 (m, 4H), 3.01 (s, 2H), 2.55-2.52 (m, 3H); ES-LCMS m/z 542.2 [M+H]⁺.

### Step 4: N-[2-[2-[2-[4-[5-(Methylsulfamoyl)-2-[[4-(trifluoromethyl)phenyl]methylamino]phenyl]imidazol-1-yl]ethoxy]ethoxy]ethyl]acetamide

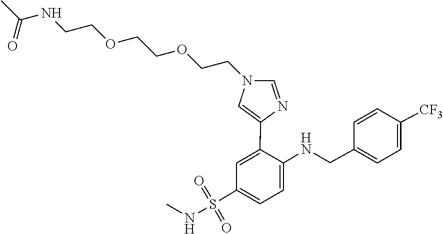 [SMILES: CNS(=O)(=O)c1ccc(NCc2ccc(C(F)(F)F)cc2)c(-c2cn(CCOCCOCCNC(C)=O)cn2)c1]

To a solution of 3-[1-[2-[2-(2-aminoethoxy)ethoxy]ethyl]imidazol-4-yl]-N-methyl-4-[[4-(trifluoromethyl)phenyl]methylamino]benzenesulfonamide (100 mg, 147.71 μmol, 80% purity, 1 eq) in DCM (3 mL) was added acetyl chloride (10.44 mg, 132.94 μmol, 9.49 μL, 0.9 eq). The mixture was stirred at 25° C. for 1 h. The solvent was removed to yield a residue which was purified by preparative HPLC (column: Phenomenex Synergi C18 150*30 mm*4 μm; mobile phase: [water (0.05% HCl)-ACN]; B %: 30%-50%, 10 min), followed by lyophilization to yield N-[2-[2-[2-[4-[5-(methylsulfamoyl)-2-[[4-(trifluoromethyl)phenyl]methylamino]phenyl]imidazol-1-yl]ethoxy]ethoxy]ethyl]acetamide (12.96 mg, 20.90 μmol, 14.2% yield, 100.0% purity, HCl) as a yellow solid. ¹H NMR (400 MHZ, DMSO-d₆) δ ppm 8.88 (s, 1H), 8.02-7.85 (m, 2H), 7.70 (d, J=8.3 Hz, 2H), 7.64 (s, 1H), 7.59 (d, J=8.1 Hz, 2H), 7.49 (d, J=8.3 Hz, 1H), 7.17-7.02 (m, 1H), 6.65 (d, J=8.8 Hz, 1H), 4.56 (s, 2H), 4.36 (s, 2H), 3.84 (t, J=4.9 Hz, 2H), 3.62-3.58 (m, 2H), 3.52 (dd, J=2.7, 5.4 Hz, 4H), 3.16 (q, J=5.8 Hz, 2H), 2.35 (d, J=4.2 Hz, 3H), 1.78 (s, 3H); ES-LCMS m/z 584.2 [M+H]⁺.

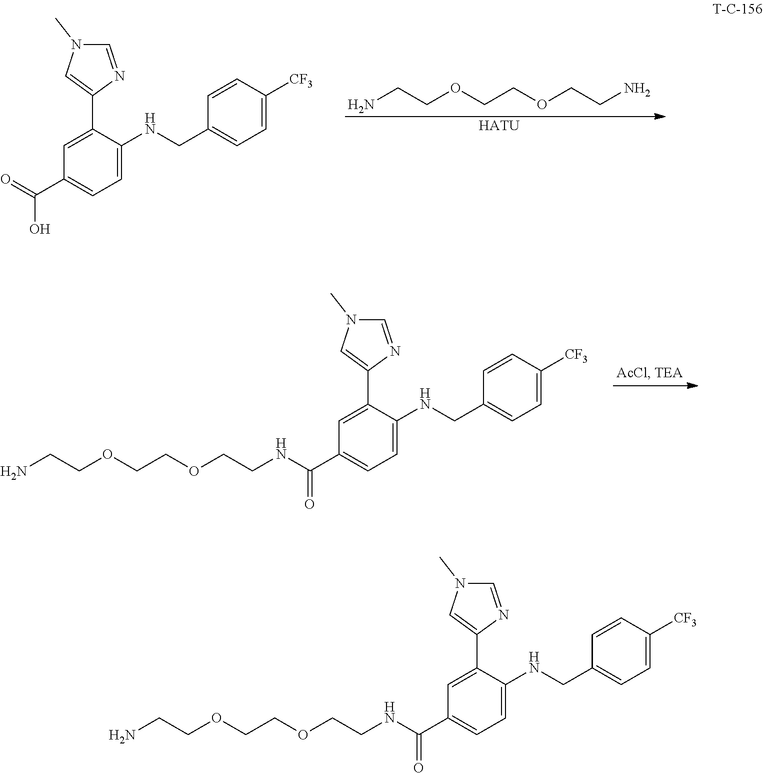

### Step 1: N-(2-(2-(2-Aminoethoxy)ethoxy)ethyl)-3-(1-methyl-1H-imidazol-4-yl)-4-((4-(trifluoromethyl)benzyl)amino)benzamide

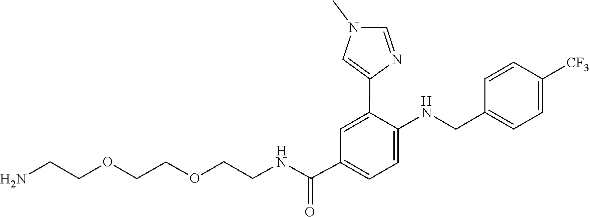 [SMILES: Cn1cnc(-c2cc(C(=O)NCCOCCOCCN)ccc2NCc2ccc(C(F)(F)F)cc2)c1]

To a solution of 3-(1-methylimidazol-4-yl)-4-[[4-(trifluoromethyl)phenyl]methylamino]benzoic acid (140 mg, 331.96 μmol, 89.0% purity, 1 eq) in DMF (25 mL) was added 2,2′-(ethane-1,2-diylbis(oxy))diethanamine (344.38 mg, 2.32 mmol, 7 eq), HATU (151.47 mg, 398.35 μmol, 1.2 eq) and N,N-diethylethanamine (100.77 mg, 995.89 μmol, 138.61 μL, 3 eq). The mixture was stirred at 25° C. for 1 h. The reaction mixture was quenched by addition of water (40 mL) and extracted with EtOAc (30 mL×3). The combined organic layers were washed with brine (20 mL), dried over Na₂SO₄, filtered and concentrated under reduced pressure to yield N-[2-[2-(2-aminoethoxy)ethoxy]ethyl]-3-(1-methylimidazol-4-yl)-4-[[4-(trifluoromethyl)phenyl]methylamino]benzamide (250 mg, crude) as a black brown solid, which was used in the next step without further purification. ES-LCMS m/z 506.2 [M+H]⁺.

### Step 2: N-(2-(2-(2-Acetamidoethoxy)ethoxy)ethyl)-3-(1-methyl-1H-imidazol-4-yl)-4-((4-(trifluoromethyl)benzyl)amino)benzamide

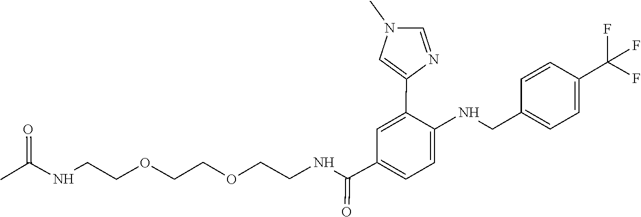 [SMILES: CC(=O)NCCOCCOCCNC(=O)c1ccc(NCc2ccc(C(F)(F)F)cc2)c(-c2cn(C)cn2)c1]

To a solution of N-[2-[2-(2-aminoethoxy)ethoxy]ethyl]-3-(1-methylimidazol-4-yl)-4-[[4-(trifluoromethyl)phenyl]methylamino]benzamide (133.40 mg, 263.88 μmol, N/A purity, 1 eq) in DCM (30 mL) was added acetyl chloride (20.71 mg, 263.88 μmol, 18.83 μL, 1 eq) and TEA (80.11 mg, 791.64 μmol, 110.19 μL, 3 eq). The mixture was stirred at 25° C. for 1 h. The reaction mixture was quenched by addition of water (50 mL) and extracted with EtOAc (50 mL×3). The combined organic layers were washed with brine (20 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 35%-65%, 10 min), followed by lyophilization to yield N-[2-[2-(2-acetamidoethoxy)ethoxy]ethyl]-3-(1-methylimidazol-4-yl)-4-[[4-(trifluoromethyl)phenyl]methylamino]benzamide (45.54 mg, 82.25 μmol, 31.1% yield, 98.9% purity) as a brown solid. ¹H NMR (400 MHZ, CDCl₃) δ ppm 8.97 (s, 1H), 7.99 (s, 1H), 7.61-7.55 (m, 2H), 7.49 (d, J=8.1 Hz, 3H), 7.41 (d, J=8.3 Hz, 1H), 7.32 (s, 1H), 6.58-6.45 (m, 2H), 6.08 (s, 1H), 4.60 (s, 2H), 3.78 (s, 3H), 3.67-3.62 (m, 6H), 3.55-3.48 (m, 4H), 3.40 (d, J=5.4 Hz, 2H), 1.94 (s, 3H); ES-LCMS m/z 548.3 [M+H]⁺.

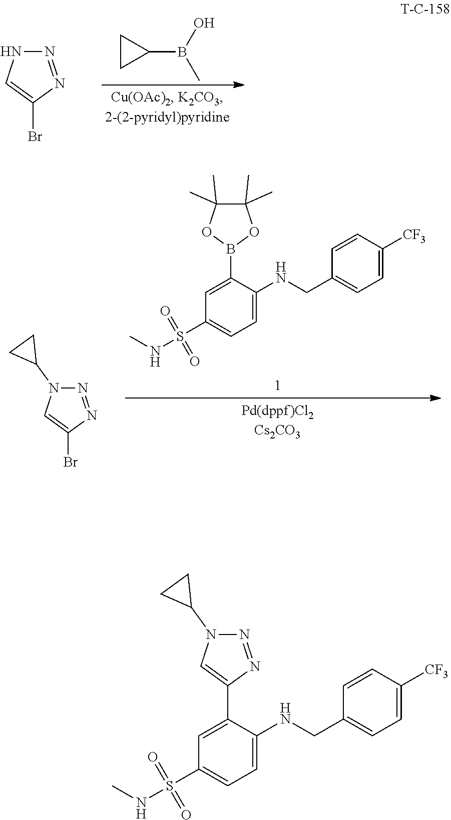

### Step 1: 4-Bromo-1-cyclopropyl-triazole

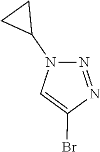

To a solution of 4-bromo-1H-triazole (250 mg, 1.69 mmol, 1 eq) in 1,2-dichloroethane (10 mL) was added 2-(2-pyridyl)pyridine (263.89 mg, 1.69 mmol, 1 eq), Cu(OAc)₂ (306.89 mg, 1.69 mmol, 1 eq), K₂CO₃ (467.05 mg, 3.38 mmol, 2 eq) and cyclopropylboronic acid (217.70 mg, 2.53 mmol, 1.5 eq). The mixture was stirred under N₂ atmosphere at 50° C. for 16 h. The reaction mixture was filtered through a pad of celite and the filtrate was concentrated to yield a residue which was purified by preparative TLC (PE/DCM=3/1, TLC: PE/DCM=3/1, R_{f}=0.60) to yield 4-bromo-1-cyclopropyl-triazole (150 mg, 398.88 μmol, 23.6% yield, 50.0% purity) as colorless oil. ES-LCMS m/z 188.1, 190.1 [M+H]⁺.

### Step 2: 3-(1-Cyclopropyltriazol-4-yl)-N-methyl-4-[[4-(trifluoromethyl)phenyl]methylamino]benzenesulfonamide

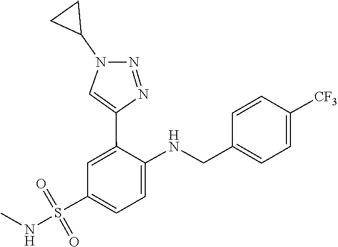 [SMILES: CNS(=O)(=O)c1ccc(NCc2ccc(C(F)(F)F)cc2)c(-c2cn(C3CC3)nn2)c1]

A mixture of 4-bromo-1-cyclopropyl-triazole (120 mg, 319.11 μmol, 50% purity, 1 eq), N-methyl-3-(4,4,5,5-tetramethyl-1,3,2-dioxaborolan-2-yl)-4-[[4-(trifluoromethyl)phenyl]methylamino]benzenesulfonamide (187.60 mg, 319.11 μmol, 80% purity, 1 eq), Pd(dppf)Cl₂ (23.35 mg, 31.91 μmol, 0.1 eq), Cs₂CO₃ (103.97 mg, 319.11 μmol, 1 eq) in 1,4-dioxane (5 mL) and H₂O (1 mL) was degassed and purged with N₂ for 3 times and the mixture was stirred under N₂ atmosphere at 100° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by preparative HPLC (column: Boston Green ODS 150*30 mm*5 μm; mobile phase: [water (0.05% NH₃·H₂O+10 mM NH₄HCO₃)-ACN]; B %: 51%-81%, 10 min), followed by lyophilization to yield 3-(1-cyclopropyltriazol-4-yl)-N-methyl-4-[[4-(trifluoromethyl)phenyl]methylamino]benzenesulfonamide (24.08 mg, 53.34 μmol, 16.7% yield, 100.0% purity) as a yellow solid. ¹H NMR (500 MHZ, DMSO-d₆) δ ppm 8.80 (s, 1H), 8.50 (t, J=6.0 Hz, 1H), 7.90 (d, J=2.1 Hz, 1H), 7.71 (d, J=8.1 Hz, 2H), 7.58 (d, J=8.1 Hz, 2H), 7.45 (dd, J=1.9, 8.8 Hz, 1H), 7.02 (q, J=5.2 Hz, 1H), 6.74 (d, J=8.9 Hz, 1H), 4.69 (d, J=6.0 Hz, 2H), 4.09 (tt, J=3.8, 7.5 Hz, 1H), 2.36 (d, J=5.2 Hz, 3H), 1.31-1.27 (m, 2H), 1.20-1.15 (m, 2H); ES-LCMS m/z 452.1 [M+H]⁺.

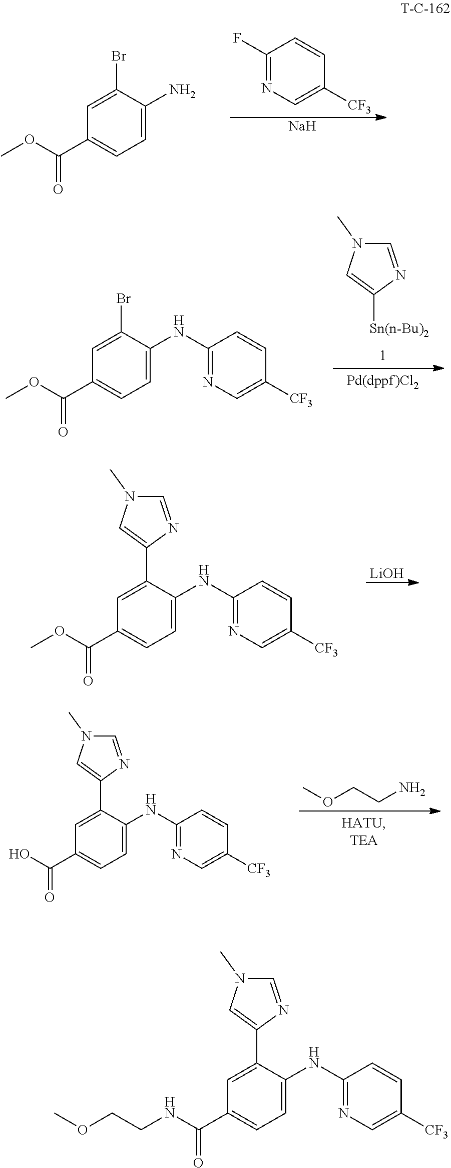

### Step 1: Methyl 3-bromo-4-[[5-(trifluoromethyl)-2-pyridyl]amino]benzoate

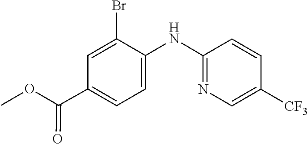 [SMILES: COC(=O)c1ccc(Nc2ccc(C(F)(F)F)cn2)c(Br)c1]

To a solution of methyl 4-amino-3-bromo-benzoate (5 g, 21.73 mmol, 1 eq) in DMF (30 mL) was added dropwise NaH (1.74 g, 43.47 mmol, 60% purity, 2 eq) at 0° C. under N₂ atmosphere. After addition, the mixture was stirred at 25° C. for 30 min. 2-Fluoro-5-(trifluoromethyl)pyridine (4.31 g, 26.11 mmol, 1.2 eq) was added. The resulting mixture was stirred at 100° C. for 12 h. The mixture was added to sat. aq. NH₄Cl at 0° C., diluted with water (300 mL) and extracted with EtOAc (300 mL×3). The organic layer was washed with brine (300 mL×2), dried over Na₂SO₄, filtered and concentrated under reduced pressure to yield a residue which was purified by flash silica gel chromatography (from PE/EtOAc=100/1 to 3/1, TLC: PE/EtOAc=3/1, R_{f}=0.72) to yield methyl 3-bromo-4-[[5-(trifluoromethyl)-2-pyridyl]amino]benzoate (1.7 g, 4.34 mmol, 19.9% yield, 95.7% purity) as a white solid. ¹H NMR (400 MHZ, CDCl₃) δ ppm 8.57 (s, 1H), 8.42 (d, J=8.6 Hz, 1H), 8.27 (d, J=2.0 Hz, 1H), 7.99 (dd, J=1.8, 8.8 Hz, 1H), 7.80 (dd, J=2.2, 8.8 Hz, 1H), 7.37 (s, 1H), 6.96 (d, J=8.6 Hz, 1H), 3.91 (s, 3H); LCMS m/z 376.7 [M+H]⁺.

### Step 2: Methyl 3-(1-methylimidazol-4-yl)-4-[[5-(trifluoromethyl)-2-pyridyl]amino]benzoate

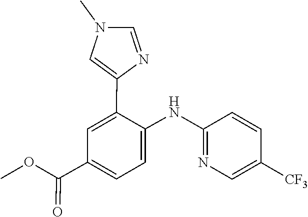 [SMILES: COC(=O)c1ccc(Nc2ccc(C(F)(F)F)cn2)c(-c2cn(C)cn2)c1]

To a solution of methyl 3-bromo-4-[[5-(trifluoromethyl)-2-pyridyl]amino]benzoate (1.7 g, 4.53 mmol, 1 eq) and tributyl-(1-methylimidazol-4-yl)stannane (2.02 g, 5.44 mmol, 1.2 eq) in DMF (20 mL) was added Pd(dppf)Cl₂ (331.58 mg, 453.00 μmol, 0.1 eq). The mixture was stirred under N₂ atmosphere at 130° C. for 12 h. TLC (PE/EtOAc=3/1, R_{f1}=0.60, R_{f2}=0.42) showed the start materials were remained and one new spot was detected. The mixture was diluted with water (100 mL) and extracted with EtOAc (100 mL×3). The organic layer was washed with brine (100 mL×2), dried over Na₂SO₄, filtered and concentrated under reduced pressure to yield a residue which was purified by flash silica gel chromatography (from PE/EtOAc=100/1 to 3/1, TLC: PE/EtOAc=3/1, R_{f}=0.19) to yield methyl 3-(1-methylimidazol-4-yl)-4-[[5-(trifluoromethyl)-2-pyridyl]amino]benzoate (1.2 g, 3.01 mmol, 66.5% yield, 94.5% purity) as a yellow solid. ¹H NMR (400 MHZ, CDCl₃) δ ppm 8.70 (d, J=8.6 Hz, 1H), 8.53 (s, 1H), 8.20 (d, J=2.0 Hz, 1H), 8.01 (s, 1H), 7.91 (dd, J=2.0, 8.6 Hz, 1H), 7.68 (dd, J=2.2, 8.8 Hz, 1H), 7.54 (d, J=0.8 Hz, 1H), 7.36 (d, J=1.2 Hz, 1H), 6.93 (d, J=8.6 Hz, 1H), 3.91 (s, 3H), 3.79 (s, 3H); LCMS m/z 377.2 [M+H]⁺.

### Step 3: 3-(1-Methylimidazol-4-yl)-4-[[5-(trifluoromethyl)-2-pyridyl]amino]benzoic acid

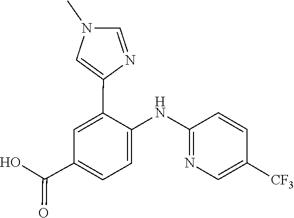 [SMILES: Cn1cnc(-c2cc(C(=O)O)ccc2Nc2ccc(C(F)(F)F)cn2)c1]

To a solution of methyl 3-(1-methylimidazol-4-yl)-4-[[5-(trifluoromethyl)-2-pyridyl]amino]benzoate (1.2 g, 3.19 mmol, 1 eq) in H₂O (10 mL), MeOH (10 mL) and THF (10 mL) was added LiOH·H₂O (669.04 mg, 15.94 mmol, 5 eq). The mixture was stirred at 25° C. for 12 h. The mixture was diluted with water (100 mL) and extracted with EtOAc (100 mL×3). The organic layer was washed with brine (100 mL×2), dried over Na₂SO₄, filtered and concentrated under reduced pressure to yield 3-(1-methylimidazol-4-yl)-4-[[5-(trifluoromethyl)-2-pyridyl]amino]benzoic acid (700 mg, 1.93 mmol, 60.6% yield, 100.0% purity) as a yellow solid. ¹H NMR (500 MHZ, DMSO-d₆) δ ppm 12.42 (s, 1H), 8.67 (d, J=8.7 Hz, 1H), 8.62 (s, 1H), 8.24 (d, J=2.0 Hz, 1H), 7.97 (dd, J=2.5, 8.8 Hz, 1H), 7.94 (s, 1H), 7.89 (d, J=1.1 Hz, 1H), 7.81 (dd, J=2.0, 8.7 Hz, 1H), 7.09 (d, J=8.7 Hz, 1H), 3.78 (s, 3H); LCMS m/z 363.2 [M+H]⁺.

### Step 4: N-(2-Methoxyethyl)-3-(1-methylimidazol-4-yl)-4-[[5-(trifluoromethyl)-2-pyridyl]amino]benzamide

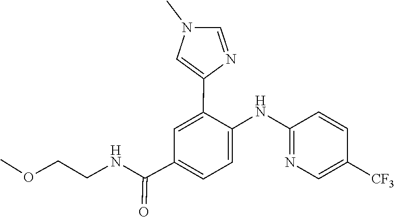 [SMILES: COCCNC(=O)c1ccc(Nc2ccc(C(F)(F)F)cn2)c(-c2cn(C)cn2)c1]

To a solution of 3-(1-methylimidazol-4-yl)-4-[[5-(trifluoromethyl)-2-pyridyl]amino]benzoic acid (100 mg, 276.01 μmol, 100% purity, 1 eq) in DMF (3 mL) was added HATU (125.94 mg, 331.21 μmol, 1.2 eq) and DIEA (107.02 mg, 828.03 μmol, 144.23 μL, 3 eq). The mixture was stirred at 25° C. for 0.5 h. 2-methoxyethanamine (103.65 mg, 1.38 mmol, 119.97 μL, 5 eq) was added and the resulting mixture was stirred 25° C. for 5 h. The reaction mixture was diluted with EtOAc (15 mL) and filtered through a pad of celite. The filtrate was concentrated under reduced pressure to yield a residue which was purified by preparative HPLC (column: Boston Prime C18 150*30 mm*5 μm; mobile phase: [water (0.05% NH₃H₂O+10 mM NH₄HCO₃)-ACN]; B %: 43%-73%, 10 min), followed by lyophilization to yield N-(2-methoxyethyl)-3-(1-methylimidazol-4-yl)-4-[[5-(trifluoromethyl)-2-pyridyl]amino]benzamide (61.73 mg, 131.86 μmol, 53.3% yield, 100.0% purity) as a white solid. ¹H NMR (400 MHZ, DMSO-d₆) δ ppm 12.25 (s, 1H), 8.60-8.55 (m, 2H), 8.48-8.44 (m, 1H), 8.19 (d, J=2.2 Hz, 1H), 7.95-7.89 (m, 2H), 7.81 (s, 1H), 7.72 (dd, J=2.0, 8.8 Hz, 1H), 7.03 (d, J=8.8 Hz, 1H), 3.78 (s, 3H), 3.51-3.42 (m, 4H), 3.28 (s, 3H); LCMS m/z 420.2 [M+H]⁺.

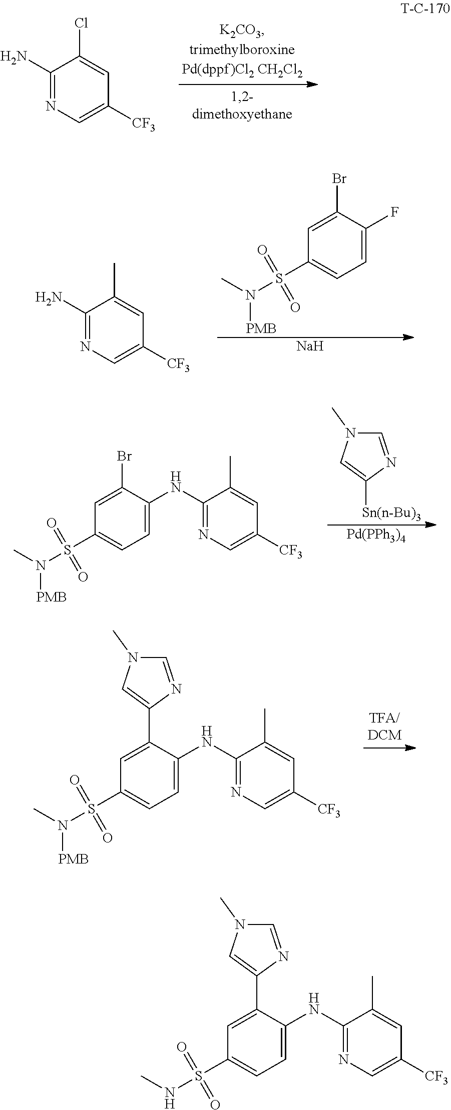

### Step 1: 3-Methyl-5-(trifluoromethyl)pyridin-2-amine

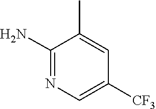

To a solution of 3-chloro-5-(trifluoromethyl)pyridin-2-amine (1 g, 5.09 mmol, 1 eq) in 1,2-dimethoxyethane (10 mL) was added K₂CO₃ (2.11 g, 15.26 mmol, 3 eq), trimethylboroxine (2.30 g, 9.16 mmol, 2.56 mL, 50%, 1.8 eq) and Pd(dppf)Cl₂·CH₂Cl₂ (415.47 mg, 508.76 μmol, 0.1 eq). The mixture was bubbled with N₂ for 3 min and stirred under microwave at 130° C. for 0.5 h. TLC (PE/EtOAc=3/1, R_{f}=0.64) indicated the starting material was consumed completely and two new spots formed. The mixture was diluted with water (30 mL) and extracted with EtOAc (30 mL×3). The combined organic phase was washed with brine (20 mL), dried over anhydrous Na₂SO₄, filtered and concentrated in vacuum to yield a residue which was purified by flash silica gel chromatography (from PE/EtOAc=100/1 to 3/1, TLC: PE/EtOAc=3/1, R_{f}=0.42) to yield 3-methyl-5-(trifluoromethyl)pyridin-2-amine (500 mg, 2.70 mmol, 53.0% yield, 95.0% purity) as a yellow solid. ¹H NMR (400 MHZ, CDCl₃) δ ppm 8.22 (s, 1H), 7.47 (s, 1H), 4.90-4.70 (m, 2H), 2.18 (s, 3H); ES-LCMS m/z 176.8 [M+H]⁺.

### Step 2: 3-Bromo-N-[(4-methoxyphenyl)methyl]-N-methyl-4-[[3-methyl-5-(trifluoromethyl)-2-pyridyl]amino]benzenesulfonamide

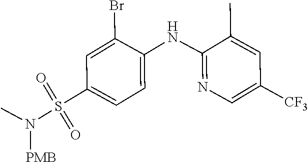

To a solution of 3-methyl-5-(trifluoromethyl)pyridin-2-amine (272.20 mg, 1.47 mmol, 95%, 1.2 eq) in DMF (10 mL) was added NaH (146.81 mg, 3.67 mmol, 60%, 3 eq) at 0° C. After being stirred for 0.5 h, 3-bromo-4-fluoro-N-[(4-methoxyphenyl)methyl]-N-methyl-benzenesulfonamide (500 mg, 1.22 mmol, 95%, 1 eq) was added. The mixture was stirred at 25 ºC for 12 h. TLC (PE/EtOAc=3/1, R_{f}=0.55) indicated the starting material was consumed completely and two new spots formed. The residue was diluted with H₂O (80 mL) and extracted with EtOAc (50 mL×3). The combined organic layers were washed with sat. aq. NaCl (30 mL), dried over Na₂SO₄, filtered and concentrated under reduced pressure to yield a residue which was purified by flash silica gel chromatography (from PE/EtOAc=100/1 to 3/1, TLC: PE/EtOAc=3/1, R_{f}=0.50) to yield 3-bromo-N-[(4-methoxyphenyl)methyl]-N-methyl-4-[[3-methyl-5-(trifluoromethyl)-2-pyridyl]amino]benzenesulfonamide (420 mg, 694.36 μmol, 56.8% yield, 90.0% purity) as a white solid. ¹H NMR (400 MHZ, CDCl₃) δ ppm 9.00 (d, J=8.8 Hz, 1H), 8.47 (s, 1H), 8.04 (d, J=2.0 Hz, 1H), 7.79 (dd, J=1.5, 8.8 Hz, 1H), 7.68 (s, 1H), 7.45 (s, 1H), 7.23 (d, J=8.3 Hz, 2H), 6.87 (d, J=8.6 Hz, 2H), 4.11 (s, 2H), 3.81 (s, 3H), 2.61 (s, 3H), 2.45 (s, 3H); ES-LCMS m/z 544.0, 546.0 [M+H]⁺.

### Step 3: N-[(4-Methoxyphenyl)methyl]-N-methyl-3-(1-methylimidazol-4-yl)-4-[[3-methyl-5-(trifluoromethyl)-2-pyridyl]amino]benzenesulfonamide

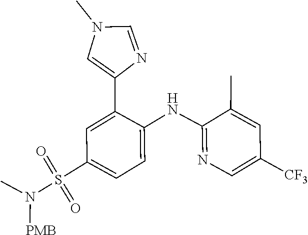

To a solution of 3-bromo-N-[(4-methoxyphenyl)methyl]-N-methyl-4-[[3-methyl-5-(trifluoromethyl)-2-pyridyl]amino]benzenesulfonamide (350 mg, 578.64 μmol, 90%, 1 eq) and tributyl-(1-methylimidazol-4-yl)stannane (438.28 mg, 1.16 mmol, 98%, 2 eq) in DMF (10 mL) was added Pd(PPh₃)+(33.43 mg, 28.93 μmol, 0.05 eq). The mixture was stirred under N₂ atmosphere at 130° C. for 12 h. TLC (PE/EtOAc=1/1, R_{f}=0.57) indicated the starting material was consumed completely and one new spot formed. The reaction mixture was concentrated under reduced pressure to yield a residue which was purified by flash silica gel chromatography (from PE/EtOAc=100/1 to 1/1, TLC: PE/EtOAc=1/1, R_{f}=0.35) to yield N-[(4-methoxyphenyl)methyl]-N-methyl-3-(1-methylimidazol-4-yl)-4-[[3-methyl-5-(trifluoromethyl)-2-pyridyl]amino]benzenesulfonamide (280 mg, 502.96 μmol, 86.9% yield, 98.0% purity) as a yellow solid. ¹H NMR (500 MHz, CDCl₃) δ ppm 11.88 (s, 1H), 9.10 (d, J=9.0 Hz, 1H), 8.43 (s, 1H), 7.96 (d, J=2.3 Hz, 1H), 7.70 (dd, J=2.2, 8.9 Hz, 1H), 7.58 (d, J=5.2 Hz, 2H), 7.37 (s, 1H), 7.24 (d, J=8.5 Hz, 2H), 6.87 (d, J=8.5 Hz, 2H), 4.10 (s, 2H), 3.81 (d, J=4.6 Hz, 6H), 2.59 (s, 3H), 2.50 (s, 3H); ES-LCMS m/z 546.2 [M+H]⁺.

### Step 4: N-Methyl-3-(1-methylimidazol-4-yl)-4-[[3-methyl-5-(trifluoromethyl)-2-pyridyl]amino]benzenesulfonamide

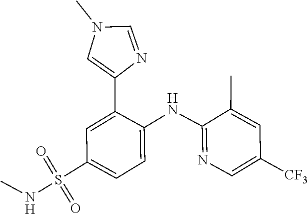 [SMILES: CNS(=O)(=O)c1ccc(Nc2ncc(C(F)(F)F)cc2C)c(-c2cn(C)cn2)c1]

A mixture of N-[(4-methoxyphenyl)methyl]-N-methyl-3-(1-methylimidazol-4-yl)-4-[[3-methyl-5-(trifluoromethyl)-2-pyridyl]amino]benzenesulfonamide (230 mg, 413.14 μmol, 98%, 1 eq) in DCM (3 mL) and TFA (1.54 g, 13.51 mmol, 1 mL, 32.69 eq) was stirred under N₂ atmosphere at 25° C. for 3 h. TLC (PE/EtOAc=1/1, R_{f}=0.51) indicated the starting material was consumed completely and one new spot formed. The reaction mixture was concentrated under reduced pressure to yield a residue. To the residue was added sat. aq. NaHCO₃ (80 mL) and the mixture was extracted with EtOAc (60 mL×3). The combined organic phase was washed with brine (20 mL), dried over anhydrous Na₂SO₄, filtered and concentrated in vacuum to yield a residue which was purified by flash silica gel chromatography (from PE/EtOAc=100/1 to 1/1, TLC: PE/EtOAc=1/1, R_{f}=0.32) to yield N-methyl-3-(1-methylimidazol-4-yl)-4-[[3-methyl-5-(trifluoromethyl)-2-pyridyl]amino]benzenesulfonamide (120.57 mg, 277.17 μmol, 67.1% yield, 97.8% purity) as a yellow solid. ¹H NMR (400 MHZ, CDCl₃) δ ppm 11.88 (s, 1H), 9.05 (d, J=8.8 Hz, 1H), 8.41 (s, 1H), 8.01 (d, J=2.4 Hz, 1H), 7.72 (dd, J=2.2, 8.8 Hz, 1H), 7.57 (d, J=9.0 Hz, 2H), 7.37 (d, J=1.0 Hz, 1H), 4.33 (q, J=5.2 Hz, 1H), 3.81 (s, 3H), 2.68 (d, J=5.6 Hz, 3H), 2.49 (s, 3H); ES-LCMS m/z 426.2 [M+H]⁺.

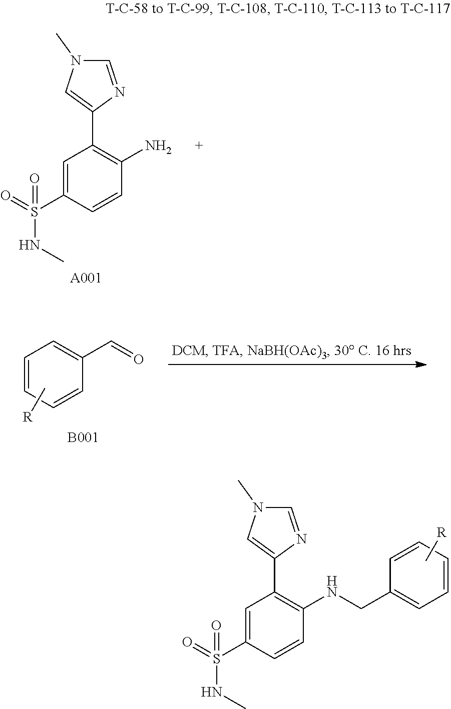

A001 (80.0 mg, 0.30 mmol, 1.0 equiv.) and B001 (0.30 mmol, 1.0 equiv.) was dissolved in DCM (3 mL) and TFA (1 mL). The mixture was added NaBH(OAc)₃ (189.9 mg, 0.90 mmol, 3.0 equiv.). The mixture was stirred at 30° C. for 16 hours. Check the reactions by LCMS. The reaction mixture was filtered and concentrated under reduced pressure to give residues. The resulting mixture was adjusted to pH 10 by the dropwise addition of saturated aqueous NH₃·H₂O. Some products were precipitated out from ACN (2 mL) and H₂O (4 mL). Or the crude product was purified by prep-HPLC to give product.

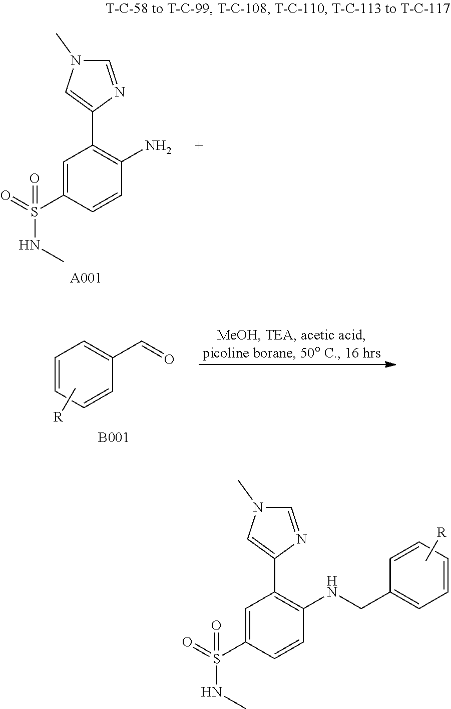

A001 (80.0 mg, 0.30 mmol, 1.0 equiv.) and B001 (0.30 mmol, 1.0 equiv.) dissolved in MeOH (3 mL), was added TEA (130.0 ul, 0.90 mmol, 3.0 equiv.) and acetic acid (300 ul). The mixture was added picoline borane (96.3 mg, 0.90 mmol, 3.0 equiv.). The mixture was stirred at 50 ºC for 16 hours. Check the reactions by LCMS. The reaction mixture was filtered and concentrated under reduced pressure to give residues. The resulting mixture was adjusted to pH 10 by the dropwise addition of saturated aqueous NH₃·H₂O. Some products were precipitated out from ACN (2 mL) and H₂O (4 mL). Or the crude product was purified by prep-HPLC to give product.

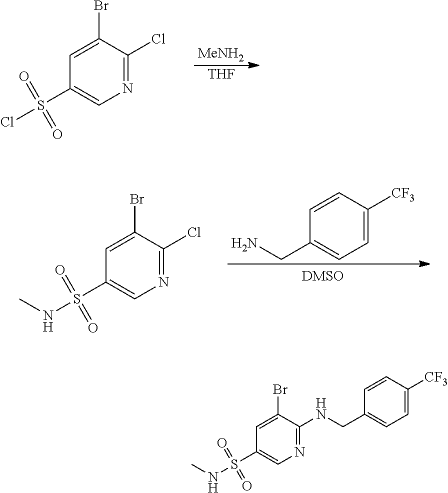 [SMILES: "MeNH2" ; O=S(=O)(Cl)c1cnc(Cl)c(Br)c1 ; CNS(=O)(=O)c1cnc(Cl)c(Br)c1 ; NCc1ccc(C(F)(F)F)cc1 ; CNS(=O)(=O)c1cnc(NCc2ccc(C(F)(F)F)cc2)c(Br)c1 ; "THF"]

### Step 1: 5-Bromo-6-chloro-N-methyl-pyridine-3-sulfonamide

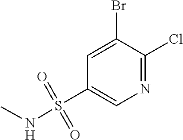

To a solution of 5-bromo-6-chloro-pyridine-3-sulfonyl chloride (2.5 g, 8.59 mmol, 1 eq) in THF (50 mL) was added MeNH₂ (1.62 g, 17.19 mmol, 33% purity, 2 eq) dropwise at −50° C. and the mixture was stirred for 1 h. TLC (PE/EtOAc=3/1, R_{f}=0.59) indicated the starting material was consumed completely and one new spot formed. The reaction was treated with water (50 mL) and extracted with EtOAc (50 mL×2). The combined organic layers were washed with brine (50 mL), dried over Na₂SO₄, filtered and concentrated under reduced pressure to yield 5-bromo-6-chloro-N-methyl-pyridine-3-sulfonamide (2.4 g, 7.73 mmol, 90% yield, 92% purity) as a white solid, which was used in the next step without further purification. ¹H NMR (400 MHZ, CDCl₃) δ ppm 8.77 (d, J=2.0 Hz, 1H), 8.35 (d, J=2.0 Hz, 1H), 4.64 (d, J=4.4 Hz, 1H), 2.76 (d, J=5.1 Hz, 4H); ES-LCMS m/z 285.0, 287.0, 289.0 [M+H]⁺.

### Step 2: 5-Bromo-N-methyl-6-[[4-(trifluoromethyl)phenyl]methylamino]pyridine-3-sulfonamide

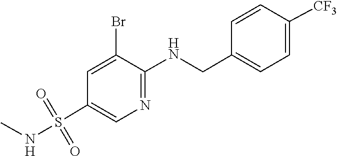 [SMILES: CNS(=O)(=O)c1cnc(NCc2ccc(C(F)(F)F)cc2)c(Br)c1]

A solution of 5-bromo-6-chloro-N-methyl-pyridine-3-sulfonamide (2.4 g, 7.73 mmol, 92% purity, 1 eq) and [4-(trifluoromethyl)phenyl]methanamine (2.71 g, 15.47 mmol, 2.20 mL, 2 eq) in DMSO (50 mL) was stirred at 140° C. for 16 h. TLC (PE/EtOAc=3/1, R_{f}=0.35) indicated the starting material was consumed completely and one new spot formed. The reaction mixture was poured into water (80 mL), extracted with EtOAc (80 mL×2). The combined organic layers were washed with brine (80 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.35) to yield 5-bromo-N-methyl-6-[[4-(trifluoromethyl)phenyl]methylamino]pyridine-3-sulfonamide (3.45 g, 7.40 mmol, 95.7% yield, 91.0% purity) as a white solid. ¹H NMR (400 MHZ, CDCl₃) δ ppm 8.51 (d, J=2.0 Hz, 1H), 8.05 (d, J=2.2 Hz, 1H), 7.61 (d, J=8.1 Hz, 2H), 7.46 (d, J=8.1 Hz, 2H), 5.92 (br s, 1H), 4.82 (d, J=5.9 Hz, 2H), 4.41 (q, J=5.1 Hz, 1H), 2.69 (d, J=5.4 Hz, 3H); ES-LCMS m/z 424.1, 426.1 [M+H]⁺.

### Step 3: N-Methyl-5-(1-methylimidazol-4-yl)-6-[[4-(trifluoromethyl)phenyl]methylamino]pyridine-3-sulfonamide

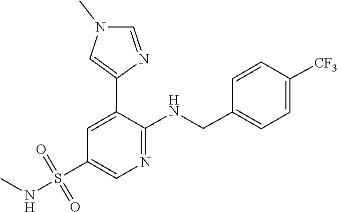 [SMILES: CNS(=O)(=O)c1cnc(NCc2ccc(C(F)(F)F)cc2)c(-c2cn(C)cn2)c1]

To a solution of 5-bromo-N-methyl-6-[[4-(trifluoromethyl)phenyl]methylamino]pyridine-3-sulfonamide (4.2 g, 9.01 mmol, 91% purity, 1 eq) and tributyl-(1-methylimidazol-4-yl)stannane (4.83 g, 11.71 mmol, 90% purity, 1.3 eq) in DMF (80 mL) was added Pd(dppf)Cl₂ (659.20 mg, 0.90 mmol, 0.1 eq). The mixture was stirred under N₂ atmosphere at 130° C. for 16 h. TLC (PE/EtOAc=1/1, R_{f}=0.24) indicated the starting material was consumed completely and one new spot formed. The solvent was removed and the residue was treated with sat. aq. KF (200 mL) and stirred for 1 h. The mixture was filtered and the filter cake was washed with EtOAc (400 mL×2). The organic phases were washed with brine (200 mL), dried over anhydrous Na₂SO₄, filtered and concentrated to yield a residue which was purified by flash silica gel chromatography (from PE/EtOAc=1/1 to 1/2, TLC: PE/EtOAc=3/1, R_{f}=0.32) to yield 3.5 g of crude product which was trituration with MeOH (30 mL) to yield N-methyl-5-(1-methylimidazol-4-yl)-6-[[4-(trifluoromethyl)phenyl]methylamino]pyridine-3-sulfonamide (2.71 g, 6.37 mmol, 70.7% yield, 100.0% purity) as a white solid. ¹H NMR (400 MHz, DMSO-d₆) δ ppm 9.88 (t, J=5.9 Hz, 1H), 8.22 (d, J=2.2 Hz, 1H), 8.01 (d, J=2.2 Hz, 1H), 7.86 (d, J=5.4 Hz, 2H), 7.68 (d, J=8.1 Hz, 2H), 7.54 (d, J=8.1 Hz, 2H), 7.18 (q, J=4.8 Hz, 1H), 4.87 (d, J=5.9 Hz, 2H), 3.75 (s, 3H), 2.41 (d, J=5.1 Hz, 3H); ES-LCMS m/z 426.2 [M+H]⁺.

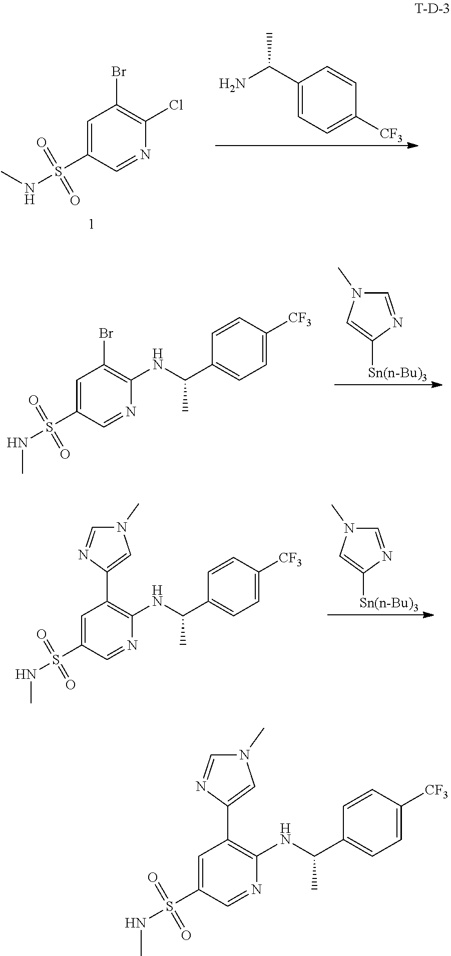

### Step 1: 5-Bromo-N-methyl-6-[[(1S)-1-[4-(trifluoromethyl)phenyl]ethyl]amino]pyridine-3-sulfonamide

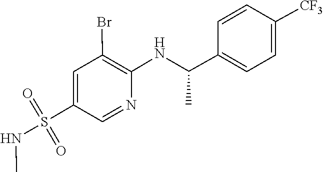 [SMILES: CNS(=O)(=O)c1cnc(N[C@@H](C)c2ccc(C(F)(F)F)cc2)c(Br)c1]

To a solution of 5-bromo-6-chloro-N-methyl-pyridine-3-sulfonamide (60 mg, 189.11 μmol, 90% purity, 1 eq) in DMSO (2 mL) was added (1R)-1-[4-(trifluoromethyl)phenyl]ethanamine (71.55 mg, 378.22 μmol, 2 eq). The mixture was stirred at 140° C. for 16 h. The mixture was diluted with water (20 mL) and extracted with EtOAc (20 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 1/1, TLC: PE/EtOAc=3/1, R_{f}=0.4) to yield 5-bromo-N-methyl-6-[[(1S)-1-[4-(trifluoromethyl)phenyl]ethyl]amino]pyridine-3-sulfonamide (83 mg, 179.92 μmol, 95.1% yield, 95.0% purity) as a light yellow solid. ¹H NMR (400 MHZ, CDCl₃) δ ppm 8.44 (d, J=2.0 Hz, 1H), 8.02 (d, J=2.0 Hz, 1H), 7.60 (d, J=8.2 Hz, 2H), 7.47 (d, J=8.6 Hz, 2H), 5.76 (d, J=6.3 Hz, 1H), 5.41-5.35 (m, 1H), 4.23 (s, 1H), 2.67 (d, J=5.5 Hz, 3H), 1.63 (d, J=7.0 Hz, 3H); ES-LCMS m/z 440.1 [M+H]⁺.

### Step 2: N-Methyl-5-(1-methylimidazol-4-yl)-6-[[(1S)-1-[4-(trifluoromethyl)phenyl]ethyl]amino]pyridine-3-sulfonamide

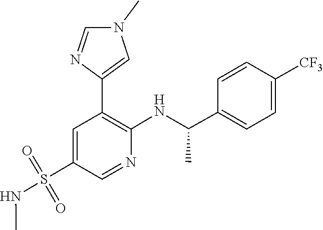 [SMILES: CNS(=O)(=O)c1cnc(N[C@@H](C)c2ccc(C(F)(F)F)cc2)c(-c2cn(C)cn2)c1]

To a solution of 5-bromo-N-methyl-6-[[(1S)-1-[4-(trifluoromethyl)phenyl]ethyl]amino]pyridine-3-sulfonamide (83 mg, 179.92 μmol, 95% purity, 1 eq) in DMF (2 mL) was added tributyl-(1-methylimidazol-4-yl)stannane (140.58 mg, 359.83 μmol, 95% purity, 2 eq) and Pd(dppf)Cl₂ (13.16 mg, 17.99 μmol, 0.1 eq). The mixture was stirred at 130° C. for 3 h. The reaction mixture was concentrated to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 46%-76%, 10 min), followed by lyophilization to yield N-methyl-5-(1-methylimidazol-4-yl)-6-[[(1S)-1-[4-(trifluoromethyl)phenyl]ethyl]amino]pyridine-3-sulfonamide (17.54 mg, 39.91 μmol, 22.1% yield, 100.0% purity) as a black brown solid. ¹H NMR (500 MHZ, CDCl₃) δ ppm 9.80 (d, J=7.3 Hz, 1H), 8.38 (d, J=2.4 Hz, 1H), 7.90 (d, J=2.3 Hz, 1H), 7.57-7.54 (m, 2H), 7.52 (d, J=7.5 Hz, 3H), 7.32 (d, J=1.1 Hz, 1H), 5.50 (m, J=7.0 Hz, 1H), 4.26 (q, J=5.3 Hz, 1H), 3.77 (s, 3H), 2.64 (d, J=5.5 Hz, 3H), 1.63 (d, J=6.9 Hz, 3H); ES-LCMS m/z 440.2 [M+H]⁺.

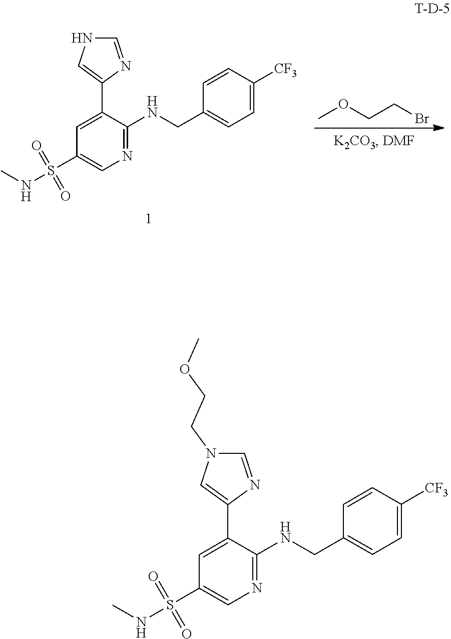

### Step 1: 5-[1-(2-Methoxyethyl)imidazol-4-yl]-N-methyl-6-[[4-(trifluoromethyl)phenyl]methylamino]pyridine-3-sulfonamide

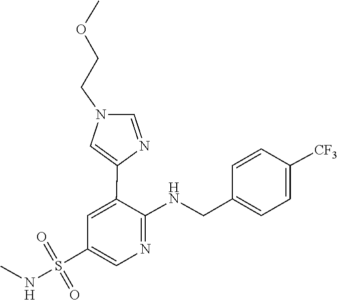 [SMILES: CNS(=O)(=O)c1cnc(NCc2ccc(C(F)(F)F)cc2)c(-c2cn(CCOC)cn2)c1]

To a solution of 5-(1H-imidazol-4-yl)-N-methyl-6-[[4-(trifluoromethyl)phenyl]methylamino]pyridine-3-sulfonamide (100 mg, 243.07 μmol, 1 eq) in DMF (2 mL) were added K₂CO₃ (67.19 mg, 486.14 μmol, 2 eq) and 1-bromo-2-methoxyethane (33.78 mg, 243.07 μmol, 22.83 μL, 1 eq). The mixture was stirred at 25° C. for 16 h. The mixture was filtered and the filtrate was concentrated to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 46%-76%, 10 min), followed by lyophilization to yield 5-[1-(2-methoxyethyl)imidazol-4-yl]-N-methyl-6-[[4-(trifluoromethyl)phenyl]methylamino]pyridine-3-sulfonamide (36.58 mg, 76.36 μmol, 31.4% yield, 98.3% purity) as a white solid. ¹H NMR (400 MHz, DMSO-d₆) δ ppm 9.91 (t, J=5.9 Hz, 1H), 8.22 (d, J=2.2 Hz, 1H), 8.02 (d, J=2.2 Hz, 1H), 7.93-7.85 (m, 2H), 7.68 (d, J=8.1 Hz, 2H), 7.55 (d, J=8.1 Hz, 2H), 7.19 (d, J=4.9 Hz, 1H), 4.87 (d, J=5.9 Hz, 2H), 4.22 (t, J=5.1 Hz, 2H), 3.67 (t, J=5.1 Hz, 2H), 3.27 (s, 3H), 2.41 (d, J=4.9 Hz, 3H); ES-LCMS m/z 470.0 [M+H]⁺.

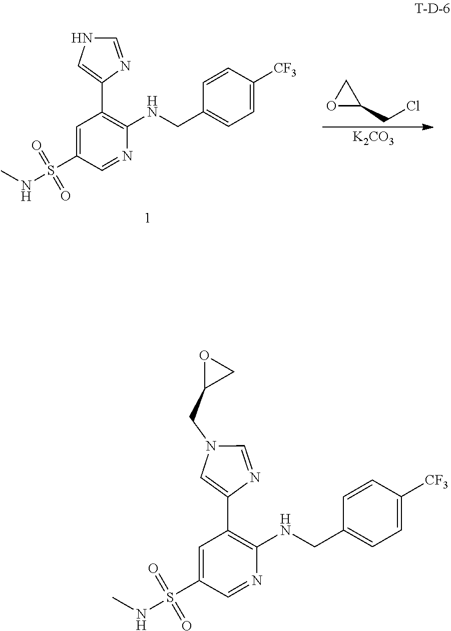

### Step 1: N-Methyl-5-[1-[[(2R)-oxiran-2-yl]methyl]imidazol-4-yl]-6-[[4-(trifluoromethyl)phenyl]methylamino]pyridine-3-sulfonamide

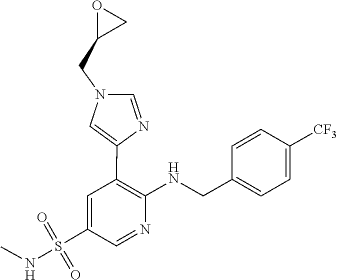 [SMILES: CNS(=O)(=O)c1cnc(NCc2ccc(C(F)(F)F)cc2)c(-c2cn(C[C@@H]3CO3)cn2)c1]

To a solution of (2S)-2-(chloromethyl)oxirane (44.98 mg, 486.14 μmol, 38.12 μL, 2 eq) and KI (80.70 mg, 486.14 μmol, 2 eq) in DMF (2 mL) was added 5-(1H-imidazol-4-yl)-N-methyl-6-[[4-(trifluoromethyl)phenyl]methylamino]pyridine-3-sulfonamide (100 mg, 243.07 μmol, 100%, 1 eq) and K₂CO₃ (67.19 mg, 486.14 μmol, 2 eq). The mixture was stirred at 60° C. for 12 h. The reaction mixture was filtered to yield the liquid which was purified by preparative HPLC (column: Agela DuraShell C18 150*25 mm*5 μm; mobile phase: [water (0.05% NH₃·H₂O+10 mM NH₄HCO₃)-ACN]; B %: 43%-73%, 10 min), followed by lyophilization to yield N-methyl-5-[1-[[(2R)-oxiran-2-yl]methyl]imidazol-4-yl]-6-[[4-(trifluoromethyl)phenyl]methylamino]pyridine-3-sulfonamide (20.51 mg, 43.88 μmol, 18.1% yield, 100.0% purity, [□]^{31.7}_{D}=+1.111 (MeOH, c=0.18 g/100 mL)) as a white solid. ¹H NMR (500 MHz, CDCl₃) δ ppm 9.77 (t, J=5.4 Hz, 1H), 8.48 (d, J=2.3 Hz, 1H), 7.98 (d, J=2.3 Hz, 1H), 7.60-7.56 (m, 3H), 7.51 (d, J=8.1 Hz, 2H), 7.46 (d, J=1.1 Hz, 1H), 4.92 (d, J=5.6 Hz, 2H), 4.45-4.35 (m, 2H), 3.97 (dd, J=6.3, 14.8 Hz, 1H), 3.30 (qd, J=3.0, 6.2 Hz, 1H), 2.93 (t, J=4.2 Hz, 1H), 2.68 (d, J=5.5 Hz, 3H), 2.57 (dd, J=2.4, 4.4 Hz, 1H); ES-LCMS m/z 468.1 [M+H]⁺.

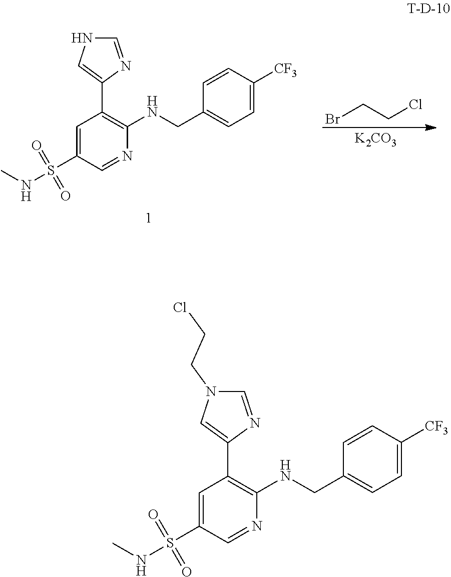

### Step 1: 5-[1-(2-Chloroethyl)imidazol-4-yl]-N-methyl-6-[[4-(trifluoromethyl)phenyl]methylamino]pyridine-3-sulfonamide

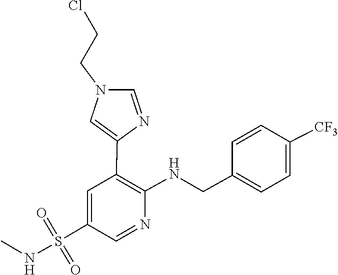 [SMILES: CNS(=O)(=O)c1cnc(NCc2ccc(C(F)(F)F)cc2)c(-c2cn(CCCl)cn2)c1]

To a solution of 5-(1H-imidazol-4-yl)-N-methyl-6-[[4-(trifluoromethyl)phenyl]methylamino]pyridine-3-sulfonamide (100 mg, 243.07 μmol, 100% purity, 1 eq) in DMF (2 mL) was added K₂CO₃ (100.78 mg, 729.22 μmol, 3 eq) and 1-bromo-2-chloro-ethane (52.29 mg, 364.61 μmol, 30.22 μL, 1.5 eq). The mixture was stirred at 25° C. for 8 h. The reaction mixture was quenched by addition H₂O (20 mL) and extracted with EtOAc (30 mL×3). The combined organic layers were washed with brine (30 mL), dried over Na₂SO₄, filtered and concentrated under reduced pressure to yield a residue which was purified by preparative HPLC (column: Welch Xtimate C18 150*25 mm*5 μm; mobile phase: [water (10 mM NH₄HCO₃)-ACN]; B %: 45%-75%, 10 min) to yield 5-[1-(2-chloroethyl)imidazol-4-yl]-N-methyl-6-[[4-(trifluoromethyl)phenyl]methylamino]pyridine-3-sulfonamide (43.57 mg, 91.94 μmol, 37.8% yield, 100.0% purity) as a white solid. ¹H NMR (400 MHZ, DMSO-d₆) δ ppm 9.86 (t, J=6.0 Hz, 1H), 8.24 (d, J=2.4 Hz, 1H), 8.01 (dd, J=2.0, 4.4 Hz, 2H), 7.96 (s, 1H), 7.69 (d, J=8.4 Hz, 2H), 7.55 (d, J=8.4 Hz, 2H), 7.20 (d, J=5.2 Hz, 1H), 4.88 (d, J=6.0 Hz, 2H), 4.42 (t, J=5.6 Hz, 2H), 4.05 (t, J=5.6 Hz, 2H), 2.41 (d, J=4.8 Hz, 3H); ES-LCMS m/z 474.1 [M+H]⁺.

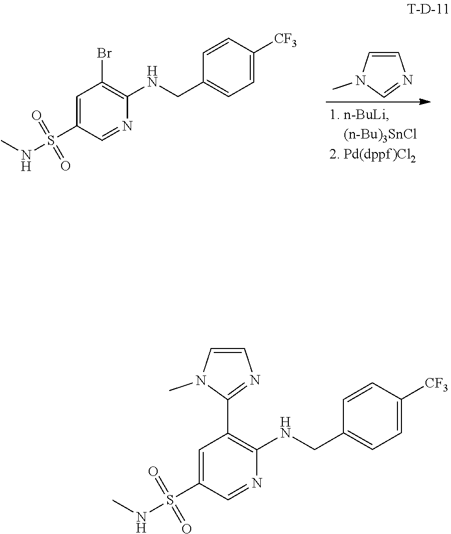

### Step 1: N-Methyl-5-(1-methylimidazol-2-yl)-6-[[4-(trifluoromethyl)phenyl]methylamino]pyridine-3-sulfonamide

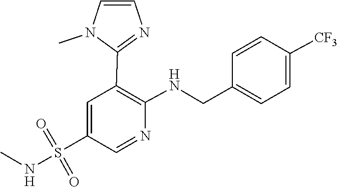 [SMILES: CNS(=O)(=O)c1cnc(NCc2ccc(C(F)(F)F)cc2)c(-c2nccn2C)c1]

To a solution of 1-methylimidazole (500 mg, 6.09 mmol, 485.44 μL, 18.13 eq) in THF (10 mL) was added n-BuLi (2.5 M, 2.38 mL, 17.68 eq) dropwise under N₂ atmosphere at −30° C. The mixture was stirred at 0° C. for 0.5 h. Tributyl(chloro)stannane (2.27 g, 6.97 mmol, 1.88 mL, 20.76 eq) was added. The mixture was stirred under N₂ atmosphere at 0° C. for 0.5 h and at 25° C. for 1 h. 5-Bromo-N-methyl-6-[[4-(trifluoromethyl)phenyl]methylamino]pyridine-3-sulfonamide (150 mg, 335.90 μmol, 95% purity, 1 eq) and Pd(dppf)Cl₂ (95.00 mg, 129.83 μmol, 3.87e-1 eq) were added. The mixture was stirred under N₂ atmosphere at 100° C. for 12 h. The mixture was diluted with water (50 mL) and KF (5 g) was added. The mixture was stirred at 25° C. for 1 h and extracted with EtOAc (50 mL×3). The organic layer was dried over Na₂SO₄, filtered and concentrated under reduced pressure to yield a residue which was purified by flash silica gel chromatography (from PE/EtOAc=100/1 to 2/1, TLC: PE/EtOAc=1/1, R_{f}=0.20) and by preparative HPLC (column: Boston Prime C18 150*30 mm*5 μm; mobile phase: [water (0.05% NH₃H₂O+10 mM NH₄HCO₃)-ACN]; B %: 47%-77%, 10 min) and lyophilized to yield N-methyl-5-(1-methylimidazol-2-yl)-6-[[4-(trifluoromethyl)phenyl]methylamino]pyridine-3-sulfonamide (25.36 mg, 58.85 μmol, 17.5% yield, 98.7% purity) as a white solid. ¹H NMR (500 MHz, CD₃OD) δ ppm 8.52 (s, 1H), 7.91 (s, 1H), 7.62 (d, J=8.0 Hz, 2H), 7.55 (d, J=8.0 Hz, 2H), 7.31 (s, 1H), 7.18 (s, 1H), 4.82 (s, 2H), 3.74 (s, 3H), 2.58 (s, 3H); ES-LCMS m/z 426.2 [M+H]⁺.

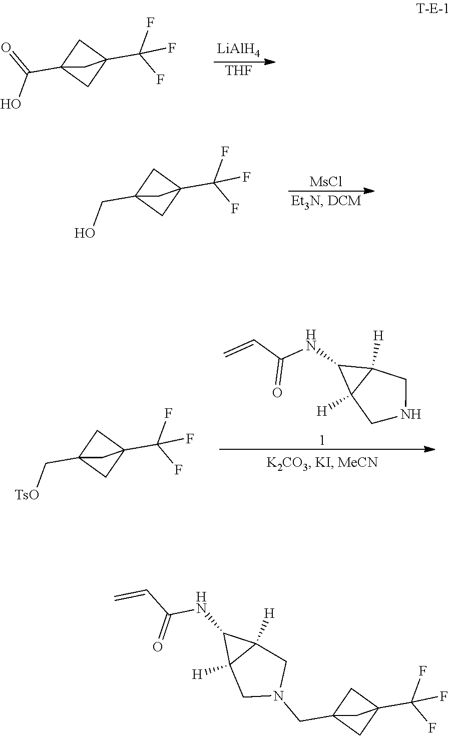

### Step 1: [3-(Trifluoromethyl)-1-bicyclo[1.1.1]pentanyl]methanol

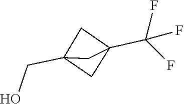 [SMILES: OCC12CC(C(F)(F)F)(C1)C2]

To a solution of LiAlH₄ (84.28 mg, 2.22 mmol, 2 eq) in THF (5 mL) was added 3-(trifluoromethyl)bicyclo[1.1.1]pentane-1-carboxylic acid (200 mg, 1.11 mmol, 1 eq) at 0° C. and stirred at 25° C. for 2 h. TLC (PE/EtOAc=1/1, R_{f}=0.60) indicated the starting material was consumed completely and one new spot formed. The mixture was quenched by 10% aq. NaOH (0.5 mL) and the precipitated solid was filtered and the filtrate was concentrated to yield [3-(trifluoromethyl)-1-bicyclo[1.1.1]pentanyl]methanol (180 mg, 975.08 μmol, 87.8% yield, 90% purity) as colorless oil, which was used in the next step without further purification. ¹H NMR (500 MHz, CDCl₃) δ ppm 3.67 (s, 2H), 1.92 (s, 6H).

### Step 2: [3-(Trifluoromethyl)-1-bicyclo[1.1.1]pentanyl]methyl methanesulfonate

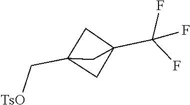 [SMILES: Cc1ccc(S(=O)(=O)OCC23CC(C(F)(F)F)(C2)C3)cc1]

To a solution of [3-(trifluoromethyl)-1-bicyclo[1.1.1]pentanyl]methanol (180 mg, 975.08 μmol, 90% purity, 1 eq) and Et₃N (197.33 mg, 1.95 mmol, 271.44 μL, 2 eq) in DCM (3 mL) was added MsCl (170 mg, 1.48 mmol, 114.86 μL, 1.52 eq) dropwise at 0° C. and the mixture stirred at 25° C. for 2 h. TLC (PE/EtOAc=1/1, R_{f}=0.67) indicated the starting material was consumed completely and one new spot formed. The mixture was quenched with sat. aq. NaHCO₃ (10 mL) and extracted with DCM (15 mL×2). The combined organic phase was dried over anhydrous Na₂SO₄, filtered and concentrated to yield [3-(trifluoromethyl)-1-bicyclo[1.1.1]pentanyl]methyl methanesulfonate (230 mg, 894.65 μmol, 91.8% yield, 95.0% purity) as a colorless gum, which was used in the next step without further purification. ¹H NMR (400 MHZ, CDCl₃) δ ppm 4.25 (s, 2H), 3.02 (s, 3H), 2.02 (s, 6H).

### Step 3: N-[(1S,5R)-3-[[3-(Trifluoromethyl)-1-bicyclo[1.1.1]pentanyl]methyl]-3-azabicyclo[3.1.0]hexan-6-yl]prop-2-enamide

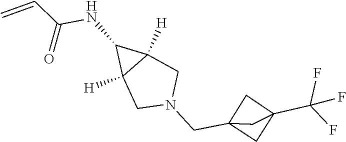 [SMILES: C=CC(=O)N[C@H]1[C@@H]2CN(CC34CC(C(F)(F)F)(C3)C4)C[C@@H]21]

To a solution of N-[(1S,5R)-3-azabicyclo[3.1.0]hexan-6-yl]prop-2-enamide (120 mg, 360.61 μmol, 80% purity, 1 eq, TFA) and [3-(trifluoromethyl)-1-bicyclo[1.1.1]pentanyl]methyl methanesulfonate (92.71 mg, 360.61 μmol, 95% purity, 1 eq) in ACN (3 mL) were added K₂CO₃ (149.52 mg, 1.08 mmol, 3 eq) and KI (5.99 mg, 36.06 μmol, 0.1 eq). The mixture was stirred at 60° C. for 16 h. The solvent was removed to yield a residue which was purified by preparative HPLC (column: Welch Xtimate C18 150*25 mm*5 μm; mobile phase: [water (10 mM NH₄HCO₃)-ACN]; B %: 48%-78%, 10 min), followed by lyophilization to yield N-[(1S,5R)-3-[[3-(trifluoromethyl)-1-bicyclo[1.1.1]pentanyl]methyl]-3-azabicyclo[3.1.0]hexan-6-yl]prop-2-enamide (18.28 mg, 60.87 μmol, 16.9% yield, 100.0% purity) as a white solid. ¹H NMR (500 MHz, CDCl₃) δ ppm 6.26 (d, J=16.9 Hz, 1H), 6.01 (dd, J=10.4, 16.9 Hz, 1H), 5.61 (d, J=10.2 Hz, 1H), 5.54 (br s, 1H), 3.17 (d, J=8.9 Hz, 2H), 3.02 (d, J=2.0 Hz, 1H), 2.51 (s, 2H), 2.37 (d, J=8.4 Hz, 2H), 1.86 (s, 6H), 1.61 (s, 2H); ES-LCMS m/z 301.2 [M+H]⁺.

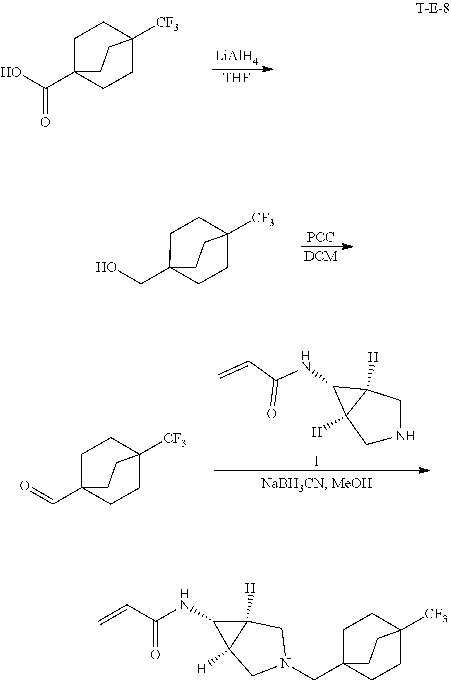

### Step 1: [4-(Trifluoromethyl)-1-bicyclo[2.2.2]octanyl]methanol

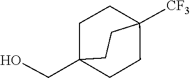 [SMILES: OCC12CCC(C(F)(F)F)(CC1)CC2]

To a solution of 4-(trifluoromethyl)bicyclo[2.2.2]octane-1-carboxylic acid (300 mg, 1.35 mmol, 1 eq) in THF (5 mL) was added LiAlH₄ (102.49 mg, 2.70 mmol, 2 eq) at 25° C. The mixture was stirred at 25° C. for 1 h. TLC (PE/EtOAc=3/1, R_{f}=0.59) indicated the starting material was consumed completely and one new spot formed. The mixture was quenched by 10% aq. NaOH (0.5 mL) and the precipitated solid was filtered and the filtrate was concentrated to yield [4-(trifluoromethyl)-1-bicyclo[2.2.2]octanyl]methanol (280 mg, 1.28 mmol, 94.6% yield, 95.0% purity) as colorless gum, which was used in the next step without further purification. ¹H NMR (400 MHZ, CDCl₃) δ ppm 3.30 (s, 2H), 1.76-1.65 (m, 6H), 1.51-1.41 (m, 6H).

### Step 2: 4-(Trifluoromethyl)bicyclo[2.2.2]octane-1-carbaldehyde

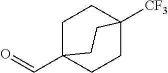 [SMILES: O=CC12CCC(C(F)(F)F)(CC1)CC2]

To a solution of [4-(trifluoromethyl)-1-bicyclo[2.2.2]octanyl]methanol (50 mg, 228.12 μmol, 95% purity, 1 eq) in DCM (3 mL) was added PCC (98.35 mg, 456.25 μmol, 2 eq). The mixture was stirred at 25° C. for 1 h. TLC (PE/EtOAc=3/1, R_{f}=0.75) indicated the starting material was consumed completely and one new spot formed. The solvent was removed to yield a residue which was purified by flash silica gel chromatography (from PE/EtOAc=2/1, TLC: PE/EtOAc=3/1, R_{f}=0.75) to yield 4-(trifluoromethyl)bicyclo[2.2.2]octane-1-carbaldehyde (50 mg, 223.08 μmol, 97.8% yield, 92.0% purity) as yellow oil. ¹H NMR (400 MHz, CDCl₃) δ ppm 9.47 (s, 1H), 1.76-1.69 (m, 12H).

### Step 3: N-[(1R,5S)-3-[[4-(Trifluoromethyl)-1-bicyclo[2.2.2]octanyl]methyl]-3-azabicyclo[3.1.0]hexan-6-yl]prop-2-enamide

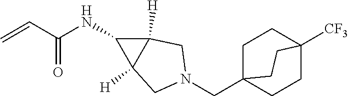 [SMILES: C=CC(=O)N[C@H]1[C@@H]2CN(CC34CCC(C(F)(F)F)(CC3)CC4)C[C@@H]21]

To a solution of N-[(1S,5R)-3-azabicyclo[3.1.0]hexan-6-yl]prop-2-enamide (120 mg, 360.61 μmol, 80% purity, 1 eq, TFA) and TEA (36.49 mg, 360.61 μmol, 50.19 μL, 1 eq) in MeOH (10 mL) was added 4-(trifluoromethyl)bicyclo[2.2.2]octane-1-carbaldehyde (47.43 mg, 211.62 μmol, 92% purity). The mixture was stirred at 25° C. for 2 h. NaBH₃CN (67.98 mg, 1.08 mmol, 3 eq) was added and the mixture was stirred at 25° C. for 16 h. The solvent was removed to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 51%-81%, 10 min), followed by lyophilization to yield N-[(1R,5S)-3-[[4-(trifluoromethyl)-1-bicyclo[2.2.2]octanyl]methyl]-3-azabicyclo[3.1.0]hexan-6-yl]prop-2-enamide (24.64 mg, 71.96 μmol, 20.0% yield, 100.0% purity) as a white solid. ¹H NMR (400 MHZ, CDCl₃) δ ppm 6.31-6.22 (m, 1H), 6.01 (dd, J=10.3, 17.1 Hz, 1H), 5.61 (dd, J=1.1, 10.1 Hz, 1H), 5.49 (br s, 1H), 3.13 (d, J=8.8 Hz, 2H), 3.02 (d, J=1.7 Hz, 1H), 2.48 (d, J=8.3 Hz, 2H), 2.13 (s, 2H), 1.68-1.59 (m, 6H), 1.49 (s, 2H), 1.43-1.32 (m, 6H); ES-LCMS m/z 343.2 [M+H]⁺.

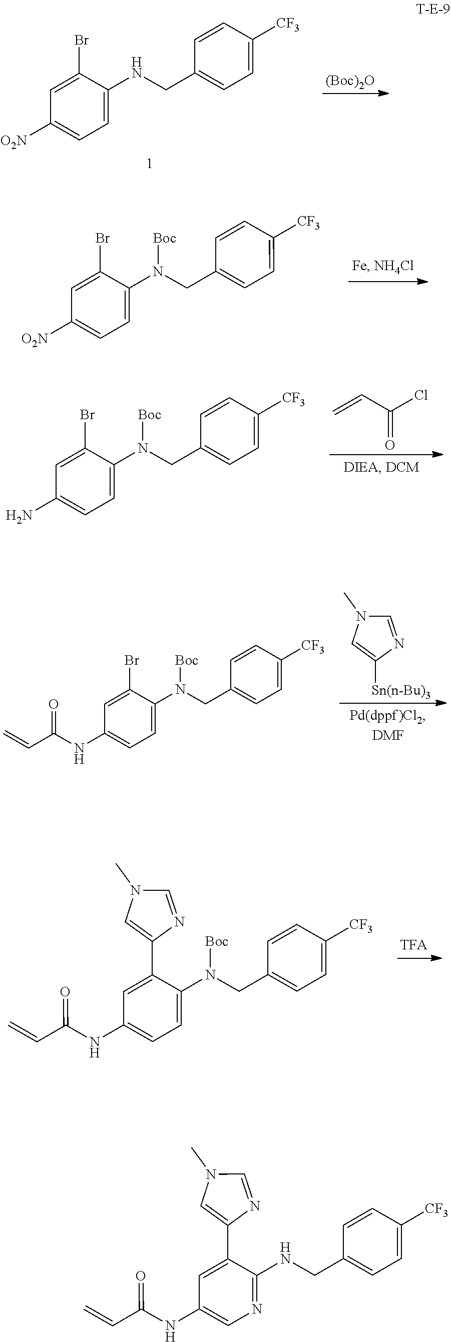

### Step 1: tert-Butyl (2-bromo-4-nitrophenyl)(4-(trifluoromethyl)benzyl)carbamate

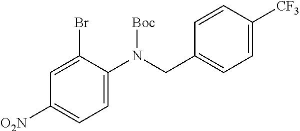 [SMILES: CC(C)(C)OC(=O)N(Cc1ccc(C(F)(F)F)cc1)c1ccc([N+](=O)[O-])cc1Br]

To a solution of 2-bromo-4-nitro-N-[[4-(trifluoromethyl)phenyl]methyl]aniline (1.10 g, 2.67 mmol, 91.1% purity, 1 eq) in DCM (10 mL) was added (Boc)₂O (1.75 g, 8.00 mmol, 1.84 mL, 3 eq) and DMAP (325.66 mg, 2.67 mmol, 1 eq). The mixture was stirred at 25° C. for 12 h. The reaction mixture was concentrated to yield a residue which was purified by flash silica gel chromatography (from PE/EtOAc=1/0 to 10/1, TLC: PE/EtOAc=10/1, R_{f}=0.51) to yield tert-butyl N-(2-bromo-4-nitro-phenyl)-N-[[4-(trifluoromethyl)phenyl]methyl]carbamate (1.15 g, 2.40 mmol, 89.9% yield, 98.7% purity) as a white solid. ¹H NMR (400 MHZ, CDCl₃) δ ppm 8.49 (br s, 1H), 8.06 (d, J=7.8 Hz, 1H), 7.57 (d, J=7.4 Hz, 2H), 7.36 (d, J=8.2 Hz, 2H), 5.20 (d, J=14.1 Hz, 1H), 4.39 (d, J=15.7 Hz, 1H), 1.57-1.40 (m, 9H); ES-LCMS m/z 375.3[M-Boc+H]⁺.

### Step 2: tert-Butyl (4-amino-2-bromophenyl)(4-(trifluoromethyl)benzyl)carbamate

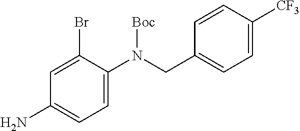 [SMILES: CC(C)(C)OC(=O)N(Cc1ccc(C(F)(F)F)cc1)c1ccc(N)cc1Br]

To a solution of tert-butyl N-(2-bromo-4-nitro-phenyl)-N-[[4-(trifluoromethyl)phenyl]methyl]carbamate (1.11 g, 2.31 mmol, 98.7%, 1 eq) in EtOH (5 mL) and H₂O (5 mL) was added Fe (646.28 mg, 11.57 mmol, 5 eq) and NH₄Cl (1.24 g, 23.15 mmol, 10 eq). The mixture was stirred at 80° C. for 1 h. The reaction mixture was quenched by addition of water (40 mL) and extracted with EtOAc (30 mL×3). The combined organic layers were washed with brine (10 mL), dried over Na₂SO₄, filtered and concentrated under reduced pressure to yield tert-butyl N-(4-amino-2-bromo-phenyl)-N-[[4-(trifluoromethyl)phenyl]methyl]carbamate (1.01 g, 2.06 mmol, 89.1% yield, 91.0% purity) as a yellow solid, which was used in the next step without further purification. ¹H NMR (400 MHZ, CDCl₃) δ ppm 7.54 (d, J=7.8 Hz, 2H), 7.39 (s, 2H), 6.95-6.88 (m, 1H), 6.58 (d, J=8.6 Hz, 1H), 6.50-6.39 (m, 1H), 5.20 (d, J=14.9 Hz, 1H), 4.27-4.20 (m, 1H), 3.72 (s, 2H), 1.38 (s, 9H); ES-LCMS m/z 391.0 [M-Boc+H]⁺.

### Step 3: tert-Butyl (4-acrylamido-2-bromophenyl)(4-(trifluoromethyl)benzyl)carbamate

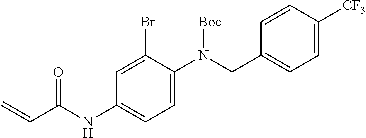 [SMILES: C=CC(=O)Nc1ccc(N(Cc2ccc(C(F)(F)F)cc2)C(=O)OC(C)(C)C)c(Br)c1]

To a solution of tert-butyl N-(4-amino-2-bromo-phenyl)-N-[[4-(trifluoromethyl)phenyl]methyl]carbamate (250 mg, 539.00 μmol, 96% purity, 1 eq) in DCM (10 mL) was added acryloyl chloride (73.18 mg, 808.49 μmol, 65.92 μL, 1.5 eq) and DIEA (139.32 mg, 1.08 mmol, 187.77 μL, 2 eq). The mixture was stirred at 25° C. for 1 h. The reaction mixture was quenched by addition of water (50 mL) and extracted with DCM (50 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.43) to yield tert-butyl N-[2-bromo-4-(prop-2-enoylamino)phenyl]-N-[[4-(trifluoromethyl)phenyl]methyl]carbamate (243 mg, 486.66 μmol, 90.2% yield, 100% purity) as colorless oil. ¹H NMR (400 MHZ, CDCl₃) δ ppm 7.60-7.53 (m, 2H), 7.40-7.29 (m, 3H), 7.26-7.18 (m, 1H), 6.92-6.75 (m, 1H), 6.46 (d, J=16.8 Hz, 1H), 6.21 (dd, J=10.6, 16.8 Hz, 1H), 5.84-5.74 (m, 1H), 5.21 (d, J=15.3 Hz, 1H), 4.30 (s, 1H), 4.26 (s, 1H), 4.13 (q, J=7.2 Hz, 1H), 2.06 (s, 1H), 1.63-1.52 (m, 9H), 1.33-1.25 (m, 2H); ES-LCMS m/z 521.1 [M+H]⁺.

### Step 4: tert-Butyl (4-acrylamido-2-(1-methyl-1H-imidazol-4-yl)phenyl)(4-(trifluoromethyl)benzyl)carbamate

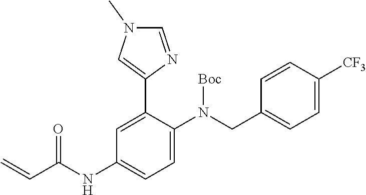 [SMILES: C=CC(=O)Nc1ccc(N(Cc2ccc(C(F)(F)F)cc2)C(=O)OC(C)(C)C)c(-c2cn(C)cn2)c1]

To a solution of tert-butyl N-[2-bromo-4-(prop-2-enoylamino)phenyl]-N-[[4-(trifluoromethyl)phenyl]methyl]carbamate (200 mg, 400.54 μmol, 100%, 1 eq) and tributyl-(1-methylimidazol-4-yl)stannane (234.73 mg, 600.82 μmol, 95%, 1.5 eq) in DMF (7 mL) was added Pd(dppf)Cl₂ (29.31 mg, 40.05 μmol, 0.1 eq). The mixture was stirred under N₂ atmosphere at 130° C. for 1 h. The reaction mixture was quenched by addition of water (50 mL) and extracted with EtOAc (50 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 4/5, TLC: PE/EtOAc=3/1, R_{f}=0.20) to yield tert-butyl N-[2-(1-methylimidazol-4-yl)-4-(prop-2-enoylamino)phenyl]-N-[[4-(trifluoromethyl)phenyl]methyl]carbamate (100 mg, 199.80 μmol, 49.8% yield, 100.0% purity) as a yellow solid. ¹H NMR (400 MHZ, CDCl₃) δ ppm 7.86 (s, 1H), 7.55-7.43 (m, 4H), 7.34 (d, J=7.8 Hz, 3H), 6.88 (br s, 1H), 6.73 (d, J=7.0 Hz, 1H), 6.45-6.35 (m, 1H), 6.25-6.14 (m, 1H), 5.76 (d, J=10.6 Hz, 1H), 5.18 (d, J=14.5 Hz, 1H), 4.18-4.07 (m, 1H), 4.11 (d, J=13.7 Hz, 1H), 3.67 (s, 3H), 1.26 (s, 9H); ES-LCMS m/z 501.2 [M+H]⁺.

### Step 5: N-(3-(1-Methyl-1H-imidazol-4-yl)-4-((4-(trifluoromethyl)benzyl)amino)phenyl)acrylamide

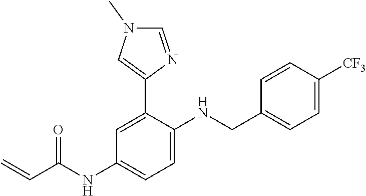 [SMILES: C=CC(=O)Nc1ccc(NCc2ccc(C(F)(F)F)cc2)c(-c2cn(C)cn2)c1]

To a solution of tert-butyl N-[2-(1-methylimidazol-4-yl)-4-(prop-2-enoylamino)phenyl]-N-[[4-(trifluoromethyl)phenyl]methyl]carbamate (100 mg, 199.80 μmol, 100%, 1 eq) in DCM (3 mL) was added TFA (3.08 g, 27.01 mmol, 2.00 mL, 135.20 eq). The mixture was stirred at 25° C. for 1 h. The reaction mixture was quenched by addition of water (30 mL) and extracted with EtOAc (30 mL×3). The combined organic layers were washed with brine (10 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 40%-70%, 10 min), followed by lyophilization to yield N-[3-(1-methylimidazol-4-yl)-4-[[4-(trifluoromethyl)phenyl]methylamino]phenyl]prop-2-enamide (10.48 mg, 26.17 μmol, 13.1% yield, 100.0% purity) as a yellow solid. ¹H NMR (400 MHZ, CDCl₃) δ ppm 8.35 (br s, 1H), 7.92 (s, 1H), 7.57-7.45 (m, 5H), 7.24 (br s, 1H), 7.08-6.94 (m, 2H), 6.47-6.36 (m, 2H), 6.26-6.14 (m, 1H), 5.71 (d, J=11.3 Hz, 1H), 4.53 (s, 2H), 3.74 (s, 3H); ES-LCMS m/z 401.2 [M+H]⁺.

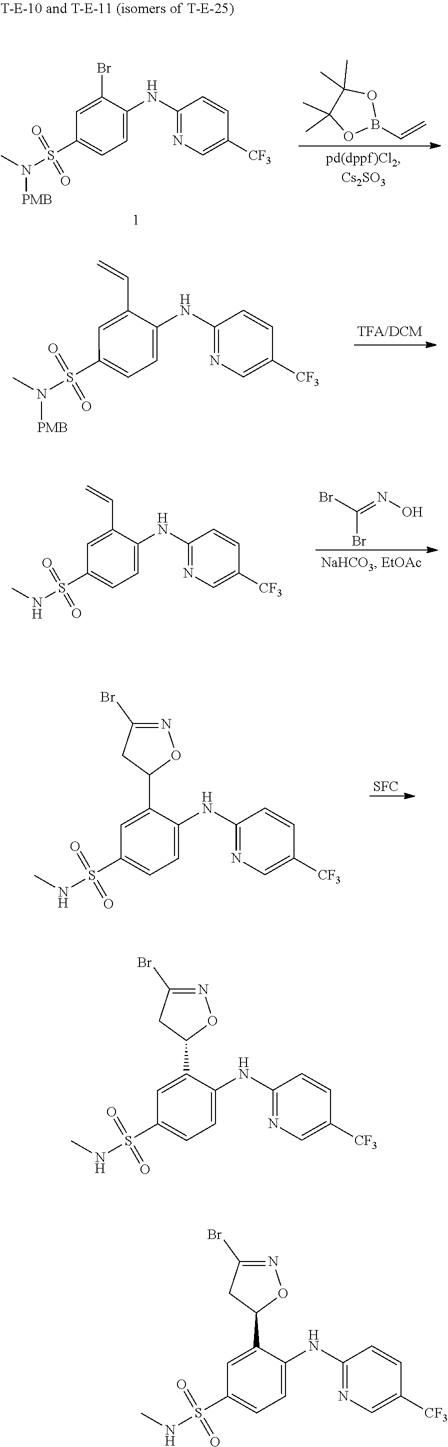

### Step 1: N-[(4-Methoxyphenyl)methyl]-N-methyl-4-[[5-(trifluoromethyl)-2-pyridyl]amino]-3-vinyl-benzenesulfonamide

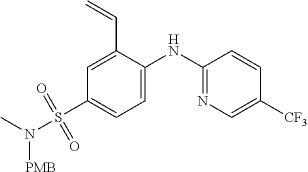

To a solution of 3-bromo-N-[(4-methoxyphenyl)methyl]-N-methyl-4-[[5-(trifluoromethyl)-2-pyridyl]amino]benzenesulfonamide (600 mg, 1.13 mmol, 1 eq) and 4,4,5,5-tetramethyl-2-vinyl-1,3,2-dioxaborolane (348.48 mg, 2.26 mmol, 383.79 μL, 2 eq) in 1,4-dioxane (10 mL) and H₂O (2 mL) was added Cs₂CO₃ (737.21 mg, 2.26 mmol, 2 eq) and Pd(dppf)Cl₂ (82.78 mg, 113.13 μmol, 0.1 eq). The mixture was stirred under N₂ atmosphere at 100° C. for 12 h. The reaction mixture was quenched by addition of water (50 mL) and extracted with EtOAc (50 mL×3). The combined organic layers were washed with brine (30 mL), dried over Na₂SO₄, filtered and concentrated under reduced pressure to yield a residue which was purified by flash silica gel chromatography (from PE/EtOAc=1/0 to 2/1, TLC: PE/EtOAc=1/1, R_{f}=0.59) to yield N-[(4-methoxyphenyl)methyl]-N-methyl-4-[[5-(trifluoromethyl)-2-pyridyl]amino]-3-vinyl-benzenesulfonamide (500 mg, 1.04 mmol, 91.6% yield, 98.9% purity) as a white solid. ¹H NMR (400 MHZ, CDCl₃) δ ppm 8.53 (s, 1H), 7.98-7.90 (m, 2H), 7.76 (dt, J=2.2, 8.9 Hz, 2H), 7.25 (d, J=8.6 Hz, 2H), 6.93-6.82 (m, 5H), 5.83 (d, J=17.6 Hz, 1H), 5.58 (d, J=11.0 Hz, 1H), 4.13 (s, 2H), 3.81 (s, 3H), 2.63 (s, 3H); ES-LCMS m/z 478.6 [M+H]⁺.

### Step 2: N-Methyl-4-[[5-(trifluoromethyl)-2-pyridyl]amino]-3-vinyl-benzenesulfonamide

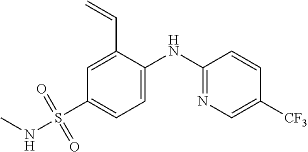 [SMILES: C=Cc1cc(S(=O)(=O)NC)ccc1Nc1ccc(C(F)(F)F)cn1]

To a solution of N-[(4-methoxyphenyl)methyl]-N-methyl-4-[[5-(trifluoromethyl)-2-pyridyl]amino]-3-vinyl-benzenesulfonamide (500 mg, 1.04 mmol, 98.9% purity, 1 eq) in DCM (2 mL) was added TFA (1.52 g, 13.37 mmol, 989.70 μL, 12.90 eq). The mixture was stirred at 25° C. for 2 h. The reaction mixture was quenched by addition of water (50 mL) and extracted with EtOAc (30 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 2/1, TLC: PE/EtOAc=2/1, R_{f}=0.49) to yield N-methyl-4-[[5-(trifluoromethyl)-2-pyridyl]amino]-3-vinyl-benzenesulfonamide (300 mg, 805.09 μmol, 77.7% yield, 95.9% purity) as colorless oil. ¹H NMR (400 MHZ, CDCl₃) δ ppm 8.50 (s, 1H), 7.97 (d, J=2.0 Hz, 1H), 7.89 (d, J=8.6 Hz, 1H), 7.77 (dt, J=2.1, 8.5 Hz, 2H), 6.94 (s, 1H), 6.90-6.80 (m, 2H), 5.83 (d, J=17.4 Hz, 1H), 5.56 (d, J=11.2 Hz, 1H), 4.51 (q, J=5.1 Hz, 1H), 2.72 (d, J=5.4 Hz, 3H); ES-LCMS m/z 358.1 [M+H]⁺.

### Step 3: 3-[(5S)-3-Bromo-4,5-dihydroisoxazol-5-yl]-N-methyl-4-[[5-(trifluoromethyl)-2-pyridyl]amino]benzenesulfonamide and 3-[(5R)-3-bromo-4,5-dihydroisoxazol-5-yl]-N-methyl-4-[[5-(trifluoromethyl)-2-pyridyl]amino]benzenesulfonamide

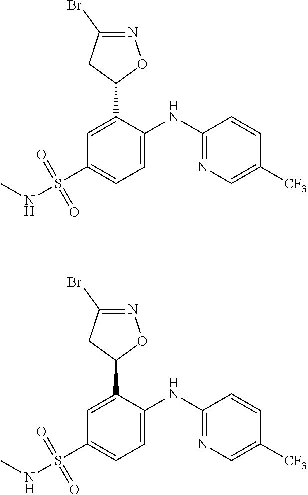 [SMILES: CNS(=O)(=O)c1ccc(Nc2ccc(C(F)(F)F)cn2)c([C@@H]2CC(Br)=NO2)c1 ; CNS(=O)(=O)c1ccc(Nc2ccc(C(F)(F)F)cn2)c([C@H]2CC(Br)=NO2)c1]

To a stirred solution of N-methyl-4-[[5-(trifluoromethyl)-2-pyridyl]amino]-3-vinyl-benzenesulfonamide (270 mg, 724.58 μmol, 95.9%, 1 eq) and dibromomethanone oxime (293.94 mg, 1.45 mmol, 2 eq) in EtOAc (10 mL) was added NaHCO₃ (608.70 mg, 7.25 mmol, 281.80 μL, 10 eq). The reaction mixture was stirred at 25° C. for 2 h. The reaction mixture was quenched by addition of water (50 mL) and extracted with EtOAc (50 mL×3). The combined organic layers were washed with brine (50 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 40%-70%, 10 min), followed by lyophilization to yield the mixture which was separated by chiral SFC column: DAICEL CHIRALPAK IG (250 mm*30 mm, 10 μm); mobile phase: [0.1% NH₃·H₂O MEOH]; B %: 40%-40%) to yield Peak 1 and Peak 2. Peak 1 was concentrated under reduced pressure to yield a residue which was dissolved in MeCN (10 mL) and H₂O (20 mL) and lyophilized to yield 3-[(5S)-3-bromo-4,5-dihydroisoxazol-5-yl]-N-methyl-4-[[5-(trifluoromethyl)-2-pyridyl]amino]benzenesulfonamide (44.51 mg, 92.87 μmol, 12.8% yield, 100.0% purity, SFC: Rₜ=1.658, ee=100%, [α]^{31.4}_{D}=−44.4 (CH₃OH, c=0.054 g/100 mL)) as a yellow solid. ¹H NMR (400 MHZ, CDCl₃) δ ppm 8.51 (s, 1H), 8.19 (d, J=8.6 Hz, 1H), 7.88 (dd, J=2.1, 8.7 Hz, 1H), 7.84-7.76 (m, 2H), 7.40 (s, 1H), 6.81 (d, J=8.8 Hz, 1H), 5.81 (t, J=11.1 Hz, 1H), 4.41 (s, 1H), 3.62-3.52 (m, 1H), 3.49-3.38 (m, 1H), 2.71 (d, J=5.4 Hz, 3H); ES-LCMS m/z 479.0 481.0 [M+H]⁺. Peak 2 was concentrated under reduced pressure to yield a residue which was dissolved in MeCN (10 mL) and H₂O (20 mL) and lyophilized to yield 3-[(5R)-3-bromo-4,5-dihydroisoxazol-5-yl]-N-methyl-4-[[5-(trifluoromethyl)-2-pyridyl]amino]benzenesulfonamide (48.81 mg, 101.84 μmol, 14.1% yield, 100.0% purity, SFC: Rₜ=1.982, ee=100%, [α]^{31.4}_{D}=+40.0 (CH₃OH, c=0.050 g/100 mL)) as a yellow solid. ¹H NMR (400 MHZ, CDCl₃) δ ppm 8.51 (s, 1H), 8.20 (d, J=8.6 Hz, 1H), 7.89 (dd, J=2.2, 8.6 Hz, 1H), 7.83-7.76 (m, 2H), 7.39 (s, 1H), 6.81 (d, J=8.8 Hz, 1H), 5.81 (t, J=11.1 Hz, 1H), 4.35 (d, J=4.9 Hz, 1H), 3.62-3.51 (m, 1H), 3.49-3.39 (m, 1H), 2.71 (d, J=5.4 Hz, 3H); ES-LCMS m/z 479.1, 481.1 [M+H]⁺.

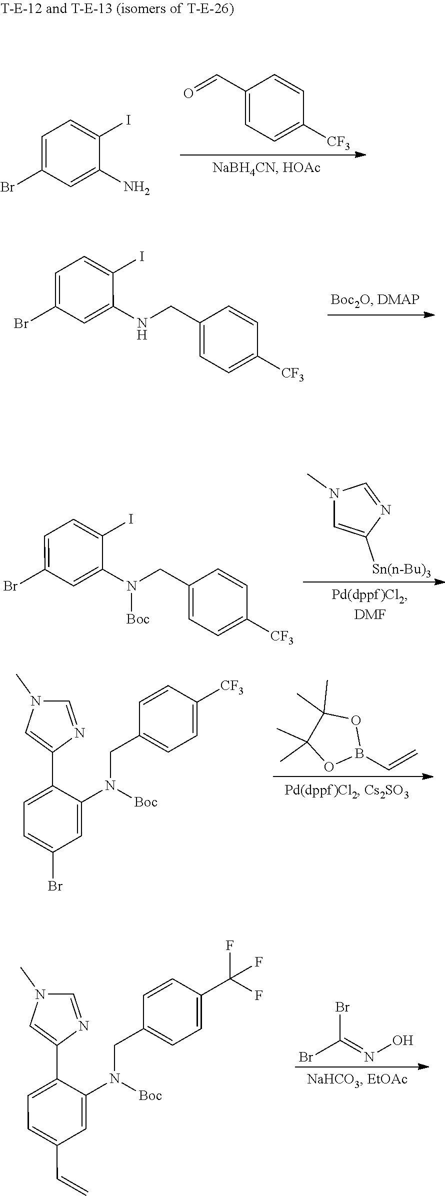

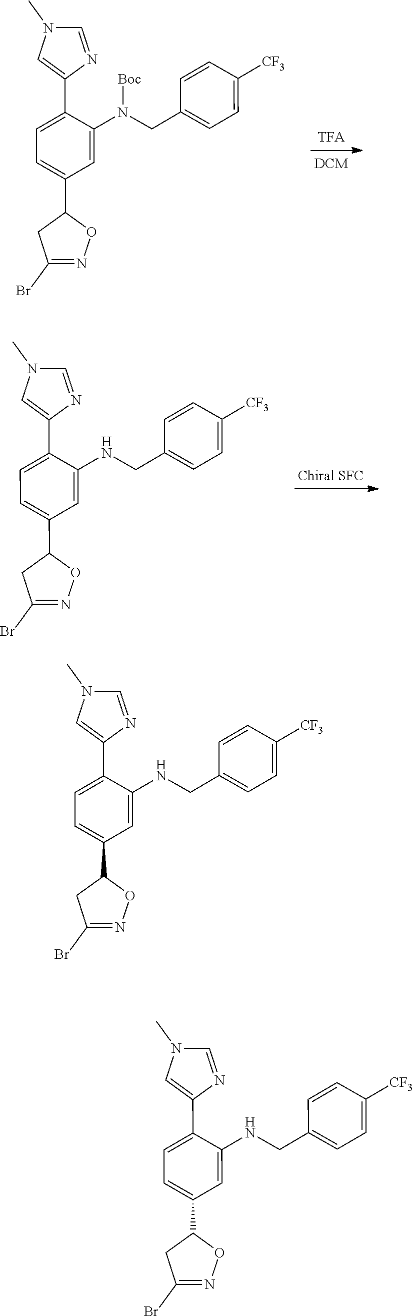

### Step 1: 5-Bromo-2-iodo-N-[[4-(trifluoromethyl)phenyl]methyl]aniline

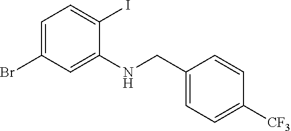 [SMILES: FC(F)(F)c1ccc(CNc2cc(Br)ccc2I)cc1]

To a solution of 5-bromo-2-iodo-aniline (2.5 g, 8.39 mmol, 1 eq) and 4-(trifluoromethyl)benzaldehyde (4.38 g, 25.17 mmol, 3.37 mL, 3 eq) in MeOH (25 mL) was added AcOH (50.39 mg, 839.16 μmol, 47.99 μL, 0.1 eq). The mixture was stirred at 60° C. for 4 h. NaBH₃CN (2.64 g, 41.96 mmol, 5 eq) was added at 25° C. The mixture was stirred at 60° C. for 12 h. The solvent was removed and the residue was quenched by addition of water (200 mL) and extracted with EtOAc (100 mL×3). The combined organic layers were washed with brine (50 mL), dried over Na₂SO₄, filtered and concentrated under reduced pressure to yield a residue which was purified by flash silica gel chromatography (from PE/EtOAc=1/0 to 5/1, TLC: PE/EtOAc=5/1, R_{f}=0.70) to yield 5-bromo-2-iodo-N-[[4-(trifluoromethyl)phenyl]methyl]aniline (1.88 g, 3.67 mmol, 43.7% yield, 89.0% purity) as yellow oil. ¹H NMR (400 MHZ, CDCl₃) δ ppm 7.64 (d, J=8.1 Hz, 2H), 7.52 (d, J=8.3 Hz, 1H), 7.47 (d, J=7.8 Hz, 2H), 6.64-6.58 (m, 2H), 4.72 (s, 1H), 4.47 (d, J=5.6 Hz, 2H); ES-LCMS m/z 455.9, 457.9 [M+H]⁺.

### Step 2: tert-Butyl N-(5-bromo-2-iodo-phenyl)-N-[[4-(trifluoromethyl)phenyl]methyl]carbamate

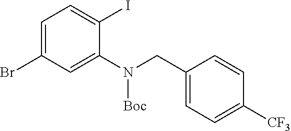 [SMILES: CC(C)(C)OC(=O)N(Cc1ccc(C(F)(F)F)cc1)c1cc(Br)ccc1I]

To a solution of 5-bromo-2-iodo-N-[[4-(trifluoromethyl)phenyl]methyl]aniline (1.88 g, 3.67 mmol, 89%, 1 eq) in THF (20 mL) was added DMAP (448.24 mg, 3.67 mmol, 1 eq) and Boc₂O (2.40 g, 11.01 mmol, 2.53 mL, 3 eq). The mixture was stirred at 20° C. for 12 h. The solvent was removed to yield a residue which was purified by flash silica gel chromatography (from PE/EtOAc=1/0 to 10/1, TLC: PE/EtOAc=10/1, R_{f}=0.8) to yield tert-butyl N-(5-bromo-2-iodo-phenyl)-N-[[4-(trifluoromethyl)phenyl]methyl]carbamate (2.23 g, crude) as a green solid. ¹H NMR (400 MHZ, CDCl₃) δ ppm 7.71 (d, J=8.3 Hz, 1H), 7.59 (d, J=7.3 Hz, 2H), 7.38 (d, J=7.8 Hz, 2H), 7.12 (d, J=7.8 Hz, 1H), 6.97 (s, 1H), 5.16 (d, J=15.2 Hz, 1H), 4.27 (d, J=14.9 Hz, 1H), 1.57 (s, 9H); ES-LCMS m/z 499.9, 421.9 [M-t-Bu+H]⁺.

### Step 3: tert-Butyl N-[5-bromo-2-(1-methylimidazol-4-yl)phenyl]-N-[[4-(trifluoromethyl)phenyl]methyl]carbamate

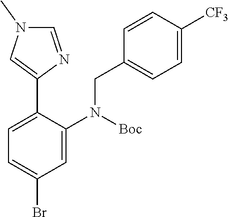 [SMILES: Cn1cnc(-c2ccc(Br)cc2N(Cc2ccc(C(F)(F)F)cc2)C(=O)OC(C)(C)C)c1]

A mixture of tert-butyl N-(5-bromo-2-iodo-phenyl)-N-[[4-(trifluoromethyl)phenyl]methyl]carbamate (1 g, 1.73 mmol, 96.3% purity, 1 eq), tributyl-(1-methylimidazol-4-yl)stannane (662.53 mg, 1.73 mmol, 97% purity, 1 eq) and Pd(dppf)Cl₂ (126.70 mg, 173.15 μmol, 0.1 eq) in DMF (10 mL) was degassed and purged with N₂ for 3 times and the mixture was stirred under N₂ atmosphere at 120° C. for 4 h. The reaction mixture was quenched by addition of water (100 mL) and extracted with EtOAc (50 mL×3). The combined organic layers were washed with brine (20 mL), dried over Na₂SO₄, filtered and concentrated under reduced pressure to yield a residue which purified by flash silica gel chromatography (from PE/EtOAc=I/O to 3/1, TLC: PE/EtOAc=3/1, R_{f}=0.40) to yield tert-butyl N-[5-bromo-2-(1-methylimidazol-4-yl)phenyl]-N-[[4-(trifluoromethyl)phenyl]methyl]carbamate (530 mg, 934.66 μmol, 54.0% yield, 90.0% purity) as black brown oil. ¹H NMR (400 MHZ, CDCl₃) δ ppm 7.97 (d, J=7.6 Hz, 1H), 7.55 (d, J=8.1 Hz, 2H), 7.46 (s, 2H), 7.35 (d, J=6.8 Hz, 2H), 6.94 (s, 1H), 6.82 (s, 1H), 5.15 (d, J=14.4 Hz, 1H), 4.18 (d, J=14.9 Hz, 1H), 3.66 (s, 3H), 1.26 (s, 9H); ES-LCMS m z 510.1, 512.1 [M+H]⁺.

### Step 4 tert-Butyl N-[2-(1-methylimidazol-4-yl)-5-vinyl-phenyl]-N-[[4-(trifluoromethyl)phenyl]methyl]carbamate

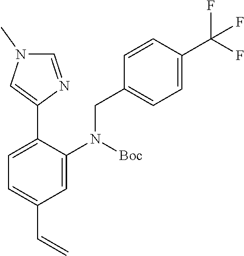 [SMILES: C=Cc1ccc(-c2cn(C)cn2)c(N(Cc2ccc(C(F)(F)F)cc2)C(=O)OC(C)(C)C)c1]

A mixture of tert-butyl N-[5-bromo-2-(1-methylimidazol-4-yl)phenyl]-N-[[4-(trifluoromethyl)phenyl]methyl]carbamate (530 mg, 934.66 μmol, 90%, 1 eq), 4,4,5,5-tetramethyl-2-vinyl-1,3,2-dioxaborolane (287.90 mg, 1.87 mmol, 317.07 μL, 2 eq), Pd(dppf)Cl₂ (68.39 mg, 93.47 μmol, 0.1 eq), Cs₂CO₃ (761.33 mg, 2.34 mmol, 2.5 eq) in 1,4-dioxane (4.5 mL) and H₂O (1.5 mL) was degassed and purged with N₂ for 3 times and the mixture was stirred under N₂ atmosphere at 100° C. for 12 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.40) to yield tert-butyl N-[2-(1-methylimidazol-4-yl)-5-vinyl-phenyl]-N-[[4-(trifluoromethyl)phenyl]methyl]carbamate (219 mg, 464.34 μmol, 49.7% yield, 97.0% purity) as yellow oil. ¹H NMR (400 MHZ, CDCl₃) δ ppm 8.05 (d, J=8.1 Hz, 1H), 7.54 (d, J=7.8 Hz, 3H), 7.47 (s, 1H), 7.38 (d, J=7.3 Hz, 3H), 6.85 (s, 1H), 6.74 (s, 1H), 6.57 (dd, J=10.9, 17.5 Hz, 1H), 5.55 (d, J=17.4 Hz, 1H), 5.18 (d, J=10.8 Hz, 1H), 5.22-5.15 (m, 1H), 3.67 (s, 3H), 1.25 (s, 9H); ES-LCMS m/z 458.1 [M+H]⁺.

### Step 5: tert-Butyl N-[2-(1-methylimidazol-4-yl)-5-vinyl-phenyl]-N-[[4-(trifluoromethyl)phenyl]methyl]carbamate

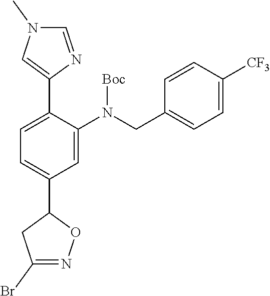 [SMILES: Cn1cnc(-c2ccc(C3CC(Br)=NO3)cc2N(Cc2ccc(C(F)(F)F)cc2)C(=O)OC(C)(C)C)c1]

To a solution of tert-butyl N-[2-(1-methylimidazol-4-yl)-5-vinyl-phenyl]-N-[[4-(trifluoromethyl)phenyl]methyl]carbamate (169 mg, 358.33 μmol, 97%, 1 eq) and dibromomethanone oxime (109.02 mg, 537.49 μmol, 1.5 eq) in EtOAc (3 mL) was added NaHCO₃ (301.02 mg, 3.58 mmol, 10 eq). The mixture was stirred at 25° C. for 4 h. The reaction mixture was quenched by addition of water (50 mL) and extracted with EtOAc (30 mL×3). The combined organic layers were washed with brine (10 mL), dried over Na₂SO₄, filtered and concentrated under reduced pressure to yield tert-butyl N-[5-(3-bromo-4,5-dihydroisoxazol-5-yl)-2-(1-methylimidazol-4-yl)phenyl]-N-[[4-(trifluoromethyl)phenyl]methyl]carbamate (248 mg, crude) as a yellow solid which was used in the next step without further purification. ¹H NMR (400 MHZ, CDCl₃) δ ppm 8.10 (s, 1H), 7.64 (s, 2H), 7.60 (d, J=8.3 Hz, 2H), 7.51-7.47 (m, 1H), 6.95-6.82 (m, 2H), 6.68 (s, 1H), 5.54 (d, J=8.8 Hz, 1H), 5.30-5.08 (m, 2H), 3.71 (s, 3H), 3.59-3.45 (m, 2H), 1.54 (s, 9H).

### Step 6: (S)-5-(3-Bromo-4,5-dihydroisoxazol-5-yl)-2-(1-methyl-1H-imidazol-4-yl)-N-(4-(trifluoromethyl)benzyl)aniline and (R)-5-(3-bromo-4,5-dihydroisoxazol-5-yl)-2-(1-methyl-1H-imidazol-4-yl)-N-(4-(trifluoromethyl)benzyl)aniline

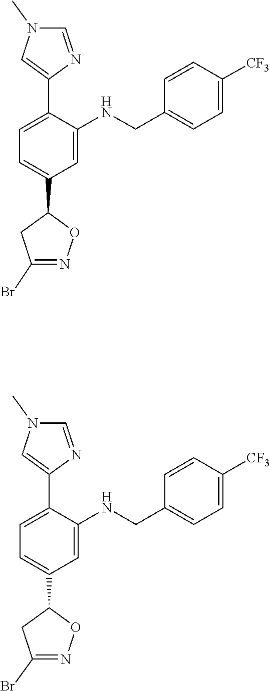 [SMILES: Cn1cnc(-c2ccc([C@@H]3CC(Br)=NO3)cc2NCc2ccc(C(F)(F)F)cc2)c1 ; Cn1cnc(-c2ccc([C@H]3CC(Br)=NO3)cc2NCc2ccc(C(F)(F)F)cc2)c1]

To a solution of tert-butyl N-[5-(3-bromo-4,5-dihydroisoxazol-5-yl)-2-(1-methylimidazol-4-yl)phenyl]-N-[[4-(trifluoromethyl)phenyl]methyl]carbamate (248 mg, 428.02 μmol, 1 eq) in DCM (3 mL) was added TFA (184.80 mg, 1.62 mmol, 120 μL, 3.79 eq). The mixture was stirred at 25° C. for 1 h. The reaction mixture was quenched by addition of water (100 mL) and extracted with EtOAc (60 mL×3). The combined organic layers were washed with brine (20 mL), dried over Na₂SO₄, filtered and concentrated under reduced pressure to yield a residue which was purified by flash silica gel chromatography (from PE/EtOAc=100/1 to 3/1, TLC: PE/EtOAc=3/1, R_{f}=0.05) to yield the compound which was separated by SFC (column: DAICEL CHIRALPAK AD (250 mm*30 mm, 10 μm); mobile phase: [0.1% NH₃H₂O ETOH]; B %: 50%-50%) to yield Peak 1 and Peak 2. Peak 1 was purified by preparative HPLC (column: Agela DuraShell C18 150*25 mm*5 μm; mobile phase: [water (0.05% NH₃H₂O+10 mM NH₄HCO₃)-ACN]; B %: 52%-82%, 10 min) and lyophilized to yield (S)-5-(3-bromo-4,5-dihydroisoxazol-5-yl)-2-(1-methyl-1H-imidazol-4-yl)-N-(4-(trifluoromethyl)benzyl)aniline (10.44 mg, 21.35 μmol, 5.0% yield, 98.5% purity, SFC: Rₜ=2.143, ee=98.2%, [α]^{32.0}_{D}=+180.0 (MeOH, c=0.02 g/100 mL)) as a white solid. ¹H NMR (400 MHZ, CDCl₃) δ ppm 8.52 (s, 1H), 7.60-7.56 (m, 2H), 7.54-7.50 (m, 2H), 7.47 (s, 1H), 7.39 (d, J=7.8 Hz, 1H), 7.18 (s, 1H), 6.61 (d, J=7.8 Hz, 1H), 6.47 (s, 1H), 5.57-5.47 (m, 1H), 4.55 (s, 2H), 3.77 (s, 3H), 3.50 (dd, J=10.9, 17.2 Hz, 1H), 3.07 (dd, J=8.9, 17.2 Hz, 1H); ES-LCMS m/z 478.8, 480.8 [M+H]⁺. Peak 2 was purified by preparative HPLC (column: Agela DuraShell C18 150*25 mm*5 μm; mobile phase: [water (0.05% NH₃H₂O+10 mM NH₄HCO₃)-ACN]; B %: 51%-81%, 10 min) and lyophilized to yield (R)-5-(3-bromo-4,5-dihydroisoxazol-5-yl)-2-(1-methyl-1H-imidazol-4-yl)-N-(4-(trifluoromethyl)benzyl)aniline (9.5 mg, 18.99 μmol, 4.4% yield, 95.9% purity, SFC: Rₜ=2.482, ee=98.8%, ee=98.8%, [α]^{32.0}_{D}=−187.5 (MeOH, c=0.016 g/100 mL)) as a white solid. ¹H NMR (400 MHZ, CDCl₃) δ ppm 8.52 (s, 1H), 7.60-7.56 (m, 2H), 7.53-7.50 (m, 2H), 7.47 (s, 1H), 7.39 (d, J=7.8 Hz, 1H), 7.18 (d, J=1.2 Hz, 1H), 6.61 (d, J=7.8 Hz, 1H), 6.47 (s, 1H), 5.52 (dd, J=8.9, 10.9 Hz, 1H), 4.55 (s, 2H), 3.77 (s, 3H), 3.50 (dd, J=10.9, 17.2 Hz, 1H), 3.07 (dd, J=8.8, 17.1 Hz, 1H); ES-LCMS m/z 479.0, 481.0 [M+H]⁺.

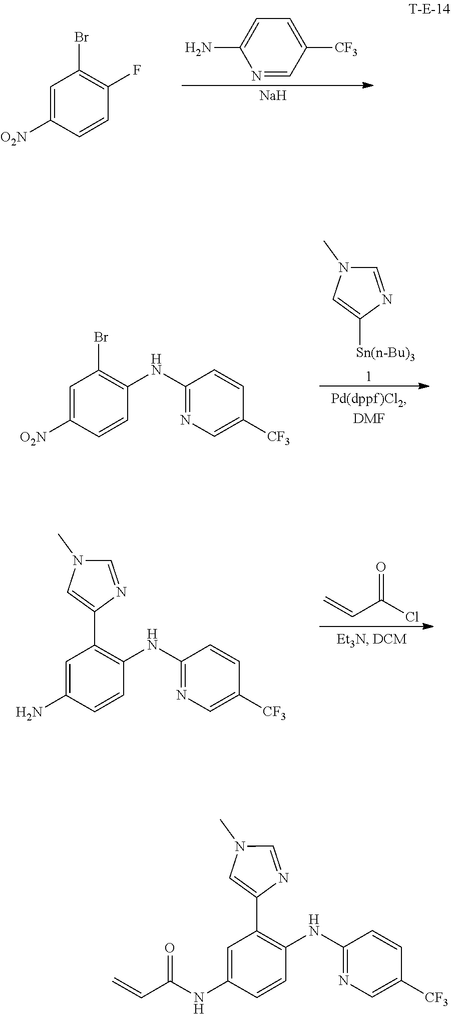

### Step 1: N-(2-Bromo-4-nitrophenyl)-5-(trifluoromethyl)pyridin-2-amine

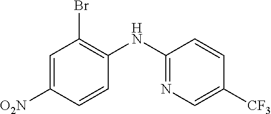 [SMILES: O=[N+]([O-])c1ccc(Nc2ccc(C(F)(F)F)cn2)c(Br)c1]

To a solution of 5-(trifluoromethyl) pyridin-2-amine (1.84 g, 11.36 mmol, 1 eq) in THF (30 mL) was added NaH (1.36 g, 34.09 mmol, 60%, 3 eq) at 0° C. The mixture was stirred for 30 min. 2-Bromo-1-fluoro-4-nitro-benzene (2.5 g, 11.36 mmol, 1 eq) was added at 0° C. and the mixture was stirred at 25° C. for 12 h. The reaction mixture was quenched with water (50 mL) and extracted with EtOAc (50 mL×3). The combined organic layers were dried over Na₂SO₄, filtered and concentrated under reduced pressure to yield a residue which was purified by flash silica gel chromatography (from PE/EtOAc=100/1 to 10/1, TLC: PE/EtOAc=5/1, R_{f}=0.65) to yield N-(2-bromo-4-nitro-phenyl)-5-(trifluoromethyl)pyridin-2-amine (850 mg, 2.00 mmol, 17.6% yield, 85.0% purity) as a yellow solid. ¹H NMR (400 MHZ, CDCl₃) δ ppm 8.76 (d, J=9.3 Hz, 1H), 8.63 (s, 1H), 8.52 (d, J=2.4 Hz, 1H), 8.24 (dd, J=2.4, 9.3 Hz, 1H), 7.87 (dd, J=2.2, 8.8 Hz, 1H), 7.54 (s, 1H), 7.00 (d, J=8.8 Hz, 1H); ES-LCMS m/z 363.9 [M+H]⁺.

### Step 2: 2-(1-Methyl-1H-imidazol-4-yl)-N¹-(5-(trifluoromethyl)pyridin-2-yl)benzene-1,4-diamine

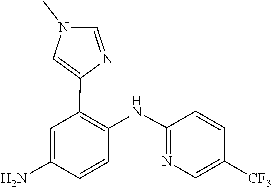 [SMILES: Cn1cnc(-c2cc(N)ccc2Nc2ccc(C(F)(F)F)cn2)c1]

To a solution of N-(2-bromo-4-nitro-phenyl)-5-(trifluoromethyl)pyridin-2-amine (700 mg, 1.93 mmol, 1 eq) and tributyl-(1-methylimidazol-4-yl)stannane (1.58 g, 3.87 mmol, 91%, 2 eq) in DMF (15 mL) was added Pd(dppf)Cl₂ (70.73 mg, 96.66 μmol, 0.05 eq) under N₂ atmosphere. The mixture was stirred under N₂ atmosphere at 130° C. for 12 h. The reaction mixture was partitioned between water (50 mL) and EtOAc (100 mL×3). The organic phase was separated, washed with brine (50 mL), dried over Na₂SO₄, filtered and concentrated under reduced pressure to yield a residue which was purified by silica gel column chromatography (from pure PE to PE/EtOAc=0/1, TLC: PE/EtOAc=0/1, R_{f}=0.34) to yield 2-(1-methylimidazol-4-yl)-N¹-[5-(trifluoromethyl)-2-pyridyl]benzene-1,4-diamine (300 mg, 648.04 μmol, 33.5% yield, 72.1% purity) as a yellow oil. ¹H NMR (400 MHZ, CDCl₃) δ ppm 9.41 (s, 1H), 8.40 (s, 1H), 7.72 (d, J=8.6 Hz, 1H), 7.59-7.43 (m, 2H), 7.27 (s, 1H), 7.18-7.01 (m, 2H), 6.74-6.57 (m, 2H), 3.71 (s, 3H); ES-LCMS m/z 334.3 [M+H]⁺.

### Step 3: N-(3-(1-Methyl-1H-imidazol-4-yl)-4-((5-(trifluoromethyl)pyridin-2-yl)amino)phenyl)acrylamide

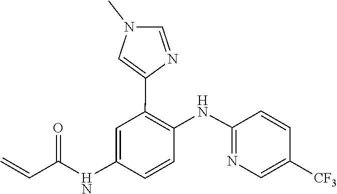 [SMILES: C=CC(=O)Nc1ccc(Nc2ccc(C(F)(F)F)cn2)c(-c2cn(C)cn2)c1]

To a solution of 2-(1-methylimidazol-4-yl)-N¹-[5-(trifluoromethyl)-2-pyridyl]benzene-1,4-diamine (230 mg, 496.83 μmol, 72%, 1 eq) and Et₃N (150.82 mg, 1.49 mmol, 207.46 μL, 3 eq) in DCM (3 mL) was added prop-2-enoyl chloride (58.46 mg, 645.88 μmol, 52.66 μL, 1.3 eq) under N₂ atmosphere at 0° C. The mixture was stirred at 25° C. for 2 h. The reaction mixture was partitioned between water (30 mL) and EtOAc (50 mL×3). The organic phase was separated, washed with brine (30 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃H₂O+10 mM NH₄HCO₃)-ACN]; B %: 38%-68%, 10 min) to yield N-[3-(1-methylimidazol-4-yl)-4-[[5-(trifluoromethyl)-2-pyridyl]amino]phenyl]prop-2-enamide (85.15 mg, 215.43 μmol, 43.4% yield, 98.2% purity) as a white solid. ¹H NMR (500 MHz, CDCl₃) δ ppm 11.30 (s, 1H), 8.44 (s, 1H), 8.38 (d, J=8.9 Hz, 1H), 8.17 (d, J=2.0 Hz, 1H), 7.74 (s, 1H), 7.60 (dd, J=2.0, 8.9 Hz, 1H), 7.47 (s, 1H), 7.18 (s, 2H), 6.82 (d, J=8.7 Hz, 1H), 6.49-6.40 (m, 1H), 6.35-6.24 (m, 1H), 5.75 (d, J=10.4 Hz, 1H), 3.68 (s, 3H); ES-LCMS m/z 388.2 [M+H]⁺.

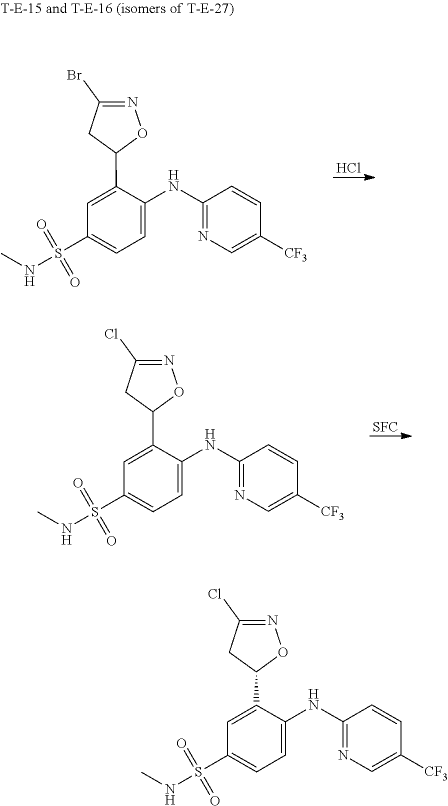 [SMILES: CNS(=O)(=O)c1ccc(Nc2ccc(C(F)(F)F)cn2)c(C2CC(Br)=NO2)c1 ; Cl ; CNS(=O)(=O)c1ccc(Nc2ccc(C(F)(F)F)cn2)c(C2CC(Cl)=NO2)c1 ; "SFC" ; CNS(=O)(=O)c1ccc(Nc2ccc(C(F)(F)F)cn2)c([C@@H]2CC(Cl)=NO2)c1]

### Step 1: 3-[(5S)-3-Chloro-4,5-dihydroisoxazol-5-yl]-N-methyl-4-[[5-(trifluoromethyl)-2-pyridyl]amino]benzenesulfonamide

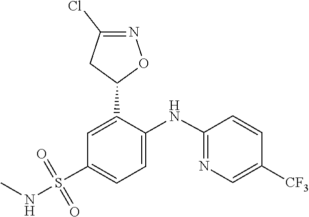 [SMILES: CNS(=O)(=O)c1ccc(Nc2ccc(C(F)(F)F)cn2)c([C@@H]2CC(Cl)=NO2)c1]

To a solution of 3-(3-bromo-4,5-dihydroisoxazol-5-yl)-N-methyl-4-[[5-(trifluoromethyl)-2-pyridyl]amino]benzenesulfonamide (900 mg, 723.35 μmol, 38.5% purity, 1 eq) in 1,4-dioxane (10 mL) was added aq. HCl (2 mL). The mixture was stirred at 40° C. for 12 h. The reaction mixture was quenched by addition of saturated NaHCO₃ (100 mL) and extracted with EtOAc (50 mL×3). The combined organic layers were washed with brine (50 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃·H₂O+10 mM NH₄HCO₃)-ACN]; B %: 40%-70%, 10 min), followed by lyophilization to yield the mixture which was separated by chiral SFC (column: column: DAICEL CHIRALPAK AS (250 mm*30 mm, 10 μm); mobile phase: [0.1% NH₃·H₂O EtOH]; B %: 25%-20%) to yield Peak 1 and Peak 2. Peak 1 was concentrated under reduced pressure to yield a residue which was dissolved in MeCN (10 mL) and H₂O (20 mL) then lyophilized to yield 3-[(5S)-3-chloro-4,5-dihydroisoxazol-5-yl]-N-methyl-4-[[5-(trifluoromethyl)-2-pyridyl]amino]benzenesulfonamide (48.36 mg, 111.22 μmol, 15.4% yield, 100.0% purity, SFC: Rₜ=1.382, ee=99.84%, [α]^{31.4}_{D}=−46.15 (CH₃OH, c=0.052 g/100 mL)) as a white solid. ¹H NMR (400 MHZ, CDCl₃) δ ppm 8.51 (s, 1H), 8.19 (d, J=8.8 Hz, 1H), 7.89 (dd, J=2.1, 8.7 Hz, 1H), 7.82-7.77 (m, 2H), 7.42 (s, 1H), 6.82 (d, J=8.8 Hz, 1H), 5.88 (t, J=11.1 Hz, 1H), 4.46-4.33 (m, 1H), 3.56-3.37 (m, 2H), 2.71 (d, J=5.1 Hz, 3H); ES-LCMS m/z 435.1, 437.1 [M+H]⁺.

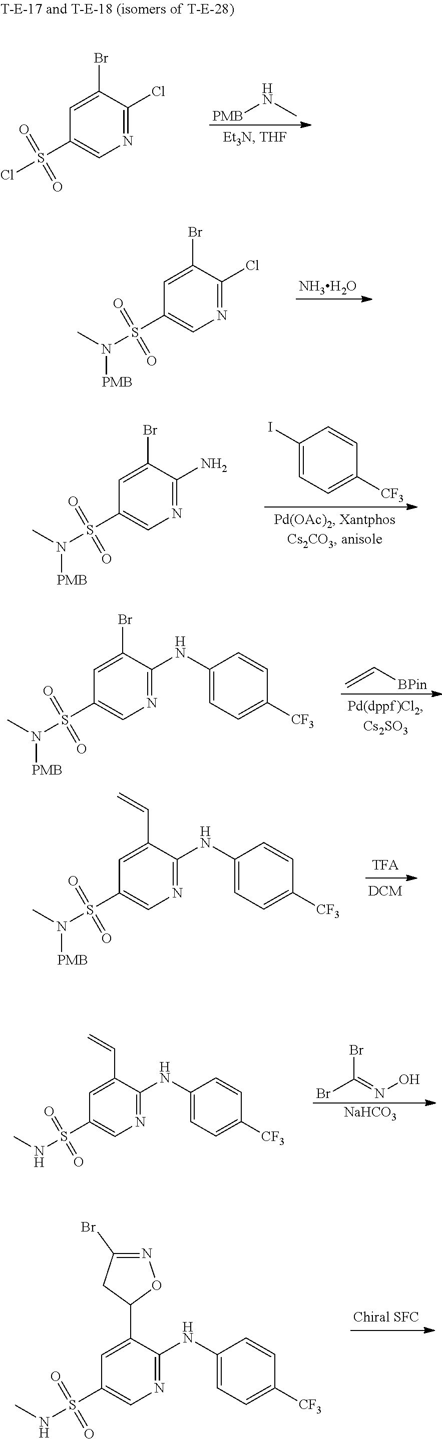

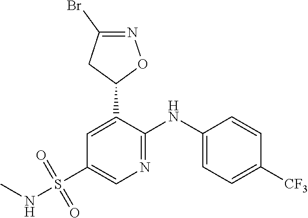 [SMILES: CNS(=O)(=O)c1cnc(Nc2ccc(C(F)(F)F)cc2)c([C@@H]2CC(Br)=NO2)c1]

### Step 1: 5-Bromo-6-chloro-N-[(4-methoxyphenyl)methyl]-N-methyl-pyridine-3-sulfonamide

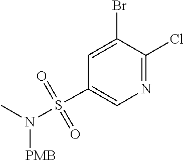

To a solution of 5-bromo-6-chloro-pyridine-3-sulfonyl chloride (1.0 g, 3.44 mmol, 1 eq) and Et₃N (695.58 mg, 6.87 mmol, 956.78 μL, 2 eq) in THF (10 mL) was added 1-(4-methoxyphenyl)-N-methyl-methanamine (571.67 mg, 3.78 mmol, 1.1 eq). The mixture was stirred at −30° C. for 1 h. TLC (PE/EtOAc=3/1, R_{f}=0.56) indicated the starting material was consumed completely and one new spot formed. The mixture was poured into water (50 mL) and the precipitated solid was filtered and dried to yield 5-bromo-6-chloro-N-[(4-methoxyphenyl)methyl]-N-methyl-pyridine-3-sulfonamide (1.1 g, 2.49 mmol, 72.6% yield, 92.0% purity) as a light yellow solid, which was used in the next step without further purification. ¹H NMR (500 MHZ, CDCl₃) δ ppm 8.71 (d, J=2.1 Hz, 1H), 8.23 (d, J=2.1 Hz, 1H), 7.21 (d, J=8.7 Hz, 2H), 6.90-6.85 (m, 2H), 4.18 (s, 2H), 3.81 (s, 3H), 2.70 (s, 3H); ES-LCMS m/z 405.0, 407.0 [M+H]⁺.

### Step 2: 6-Amino-5-bromo-N-[(4-methoxyphenyl)methyl]-N-methyl-pyridine-3-sulfonamide

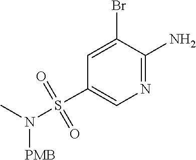

To a solution of 5-bromo-6-chloro-N-[(4-methoxyphenyl)methyl]-N-methyl-pyridine-3-sulfonamide (950 mg, 2.15 mmol, 92% purity, 1 eq) in THF (5 mL) was added NH₃·H₂O (1.48 mL, 28% purity, 5 eq). The mixture was stirred under microwave at 100° C. for 12 h. TLC (PE/EtOAc=1/1, R_{f}=0.31) indicated the starting material was consumed completely and one new spot formed. The solvent was removed to yield 6-amino-5-bromo-N-[(4-methoxyphenyl)methyl]-N-methyl-pyridine-3-sulfonamide (860 mg, 2.12 mmol, 98.2% yield, 95.0% purity) as a yellow solid, which was used in the next step without further purification. ¹H NMR (500 MHZ, DMSO-d₆) δ ppm 8.33 (d, J=2.0 Hz, 1H), 7.99 (d, J=2.0 Hz, 1H), 7.31 (br s, 2H), 7.23 (d, J=8.4 Hz, 3H), 6.92 (d, J=8.5 Hz, 2H), 3.75 (s, 3H).

### Step 3: 5-Bromo-N-[(4-methoxyphenyl)methyl]-N-methyl-6-[4-(trifluoromethyl)anilino]pyridine-3-sulfonamide

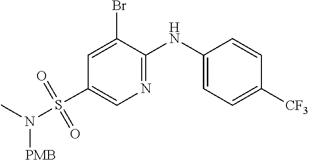

To a solution of 6-amino-5-bromo-N-[(4-methoxyphenyl)methyl]-N-methyl-pyridine-3-sulfonamide (510 mg, 1.25 mmol, 95% purity, 1 eq) and 1-iodo-4-(trifluoromethyl)benzene (409.42 mg, 1.51 mmol, 221.31 μL, 1.2 eq) in anisole (20 mL) were added Pd(OAc)₂ (42.24 mg, 188.15 μmol, 0.15 eq), xantphos (72.58 mg, 125.43 μmol, 0.1 eq) and Cs₂CO₃ (613.02 mg, 1.88 mmol, 1.5 eq). The mixture was stirred under N₂ atmosphere at 130° C. for 16 h. The solvent was removed and the residue was treated with EtOAc (30 mL). The mixture was filtered and the filtrate was concentrated to yield a residue which was purified by flash silica gel chromatography (from PE/EtOAc=1/0 to 3/1, TLC: PE/EtOAc=3/1, R_{f}=0.57) to yield 5-bromo-N-[(4-methoxyphenyl)methyl]-N-methyl-6-[4-(trifluoromethyl)anilino]pyridine-3-sulfonamide (587 mg, 1.05 mmol, 83.8% yield, 95.0% purity) as a yellow solid. ¹H NMR (500 MHZ, CDCl₃) δ ppm 8.61 (d, J=1.8 Hz, 1H), 8.12 (d, J=1.8 Hz, 1H), 7.81 (d, J=8.5 Hz, 2H), 7.64 (d, J=8.4 Hz, 2H), 7.51 (s, 1H), 7.23 (d, J=8.5 Hz, 2H), 6.88 (d, J=8.5 Hz, 2H), 4.14 (s, 2H), 3.81 (s, 3H), 2.65 (s, 3H); ES-LCMS m/z 530.0, 532.0 [M+H]⁺.

### Step 4: N-[(4-Methoxyphenyl)methyl]-N-methyl-6-[4-(trifluoromethyl)anilino]-5-vinyl-pyridine-3-sulfonamide

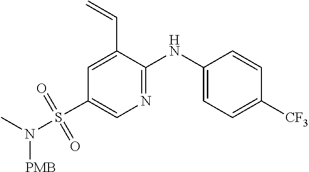

To solution a of 5-bromo-N-[(4-methoxyphenyl)methyl]-N-methyl-6-[4-(trifluoromethyl)anilino]pyridine-3-sulfonamide (587 mg, 1.05 mmol, 95% purity, 1 eq) and 4,4,5,5-tetramethyl-2-vinyl-1,3,2-dioxaborolane (323.88 mg, 2.10 mmol, 356.70 μL, 2 eq) in 1,4-dioxane (18 mL) and H₂O (3 mL) were added Pd(dppf)Cl₂ (76.94 mg, 105.15 μmol, 0.1 eq) and Cs₂CO₃ (685.17 mg, 2.10 mmol, 2 eq). The mixture was stirred under N₂ atmosphere at 90° C. for 16 h. The solvent was removed and the residue was treated with EtOAc (20 mL). The mixture was filtered and the filtrate was concentrated to yield a residue which was purified by flash silica gel chromatography (from PE/EtOAc=1/0 to 3/1, TLC: PE/EtOAc=3/1, R_{f}=0.49) to yield N-[(4-methoxyphenyl)methyl]-N-methyl-6-[4-(trifluoromethyl)anilino]-5-vinyl-pyridine-3-sulfonamide (350 mg, 732.99 μmol, 69.7% yield, 100.0% purity) as a yellow solid. ¹H NMR (500 MHZ, CDCl₃) δ ppm 8.62 (d, J=2.3 Hz, 1H), 7.89 (d, J=2.3 Hz, 1H), 7.75 (d, J=8.5 Hz, 2H), 7.61 (d, J=8.5 Hz, 2H), 7.23 (d, J=8.5 Hz, 2H), 6.87 (d, J=8.5 Hz, 3H), 6.75 (dd, J=11.1, 17.3 Hz, 1H), 5.84 (d, J=17.2 Hz, 1H), 5.71 (d, J=11.1 Hz, 1H), 4.13 (s, 2H), 3.80 (s, 3H), 2.63 (s, 3H); ES-LCMS m/z 478.2 [M+H]⁺.

### Step 5: N-Methyl-6-[4-(trifluoromethyl)anilino]-5-vinyl-pyridine-3-sulfonamide

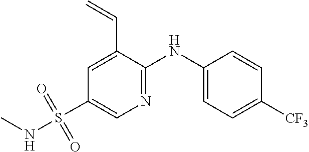 [SMILES: C=Cc1cc(S(=O)(=O)NC)cnc1Nc1ccc(C(F)(F)F)cc1]

To a solution of N-[(4-methoxyphenyl)methyl]-N-methyl-6-[4-(trifluoromethyl)anilino]-5-vinyl-pyridine-3-sulfonamide (300 mg, 628.27 μmol, 100% purity, 1 eq) in DCM (5 mL) was added TFA (1.5 mL). The mixture was stirred at 25° C. for 16 h. TLC (PE/EtOAc=1/1, R_{f}=0.58) indicated the starting material was consumed completely and one new spot formed. The mixture was concentrated to yield N-methyl-6-[4-(trifluoromethyl)anilino]-5-vinyl-pyridine-3-sulfonamide (330 mg, 616.07 μmol, 98.1% yield, 88.0% purity, TFA) as an off-white solid, which was used in the next step without further purification. ¹H NMR (500 MHZ, CD₃OD) δ ppm 8.50 (d, J=2.3 Hz, 1H), 8.09 (d, J=2.1 Hz, 1H), 7.85 (d, J=8.5 Hz, 2H), 7.62 (d, J=8.5 Hz, 2H), 7.05 (dd, J=10.9, 17.2 Hz, 1H), 5.91 (d, J=17.1 Hz, 1H), 5.62 (d, J=11.0 Hz, 1H), 2.59 (s, 3H).

### Step 6: 5-(3-Bromo-4,5-dihydroisoxazol-5-yl)-N-methyl-6-[4-(trifluoromethyl)anilino]pyridine-3-sulfonamide

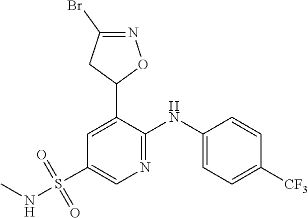 [SMILES: CNS(=O)(=O)c1cnc(Nc2ccc(C(F)(F)F)cc2)c(C2CC(Br)=NO2)c1]

To a solution of N-methyl-6-[4-(trifluoromethyl)anilino]-5-vinyl-pyridine-3-sulfonamide (330 mg, 616.07 μmol, 88% purity, 1 eq, TFA) and dibromomethanone oxime (249.92 mg, 1.23 mmol, 2 eq) in EtOAc (20 mL) was added NaHCO₃ (517.54 mg, 6.16 mmol, 10 eq). The mixture was stirred at 25° C. for 3 h. TLC (PE/EtOAc=1/1, R_{f}=0.78) indicated the starting material was consumed completely and one new spot formed. The mixture was filtered and the filtrate was concentrated to yield a residue which was purified by flash silica gel chromatography (from PE/EtOAc=1/0 to 2/1, TLC: PE/EtOAc=1/1, R_{f}=0.78) to yield 5-(3-bromo-4,5-dihydroisoxazol-5-yl)-N-methyl-6-[4-(trifluoromethyl)anilino]pyridine-3-sulfonamide (290 mg, 574.83 μmol, 93.3% yield, 95.0% purity) as a yellow solid. ¹H NMR (400 MHz, CDCl₃) δ ppm 8.71 (d, J=2.2 Hz, 1H), 7.85 (d, J=2.0 Hz, 1H), 7.74-7.66 (m, 2H), 7.64-7.57 (m, 2H), 5.77 (t, J=11.2 Hz, 1H), 4.39 (q, J=5.4 Hz, 1H), 4.12 (q, J=7.1 Hz, 1H), 3.56 (dd, J=1.8, 11.4 Hz, 2H), 2.71 (d, J=5.4 Hz, 3H); ES-LCMS m/z 479.0, 481.0 [M+H]⁺.

### Step 7: (S)-5-(3-Bromo-4,5-dihydroisoxazol-5-yl)-N-methyl-6-((4-(trifluoromethyl)phenyl)amino)pyridine-3-sulfonamide

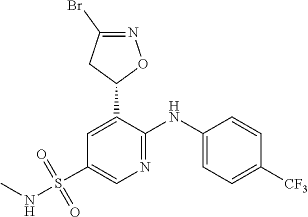 [SMILES: CNS(=O)(=O)c1cnc(Nc2ccc(C(F)(F)F)cc2)c([C@@H]2CC(Br)=NO2)c1]

The compound 5-(3-bromo-4,5-dihydroisoxazol-5-yl)-N-methyl-6-[4-(trifluoromethyl)anilino]pyridine-3-sulfonamide (100 mg, 198.22 μmol, 95% purity, 1 eq) was separated by chiral SFC (column: DAICEL CHIRALPAK AD (250 mm*30 mm, 10 μm); mobile phase: [0.1% NH₃H₂O EtOH]; B %: 30%-30%) to yield Peak 1 and Peak 2. Peak 1 was concentrated under reduced pressure to yield a residue which was dissolved in MeCN (10 mL) and H₂O (20 mL) and lyophilized to yield (S)-5-(3-bromo-4,5-dihydroisoxazol-5-yl)-N-methyl-6-((4-(trifluoromethyl)phenyl)amino)pyridine-3-sulfonamide (32.79 mg, 68.42 μmol, 34.5% yield, 100.0% purity, SFC: Rₜ=1.372, ee=100%, [□]28.6_{D}=−5.00 (MeOH, c=0.08 g/100 mL) as a white solid. ¹H NMR (500 MHZ, CDCl₃) δ ppm 8.71 (d, J=2.1 Hz, 1H), 7.85 (d, J=2.1 Hz, 1H), 7.72-7.65 (m, 3H), 7.64-7.58 (m, 2H), 5.77 (t, J=11.2 Hz, 1H), 4.44 (q, J=5.2 Hz, 1H), 3.62-3.51 (m, 2H), 2.71 (d, J=5.3 Hz, 3H); ES-LCMS m/z 478.9, 480.9 [M+H]⁺ and Peak 2 was concentrated under reduced pressure to yield a residue which was dissolved in MeCN (10 mL) and H₂O (20 mL) and lyophilized to yield (R)-5-(3-bromo-4,5-dihydroisoxazol-5-yl)-N-methyl-6-((4-(trifluoromethyl)phenyl)amino)pyridine-3-sulfonamide (31.64 mg, 66.02 μmol, 33.3% yield, 100.0% purity, SFC: Rₜ=1.596, ee=100%, [□]28.8_{D}=+6.67 (MeOH, c=0.09 g/100 mL) as a white solid. ¹H NMR (500 MHZ, CDCl₃) δ ppm 8.70 (d, J=2.3 Hz, 1H), 7.86 (d, J=2.1 Hz, 1H), 7.72-7.65 (m, 3H), 7.63-7.59 (m, 2H), 5.77 (t, J=11.1 Hz, 1H), 4.48 (q, J=5.3 Hz, 1H), 3.62-3.51 (m, 2H), 2.70 (d, J=5.3 Hz, 3H); ES-LCMS m/z 479.0, 481.0 [M+H]⁺.

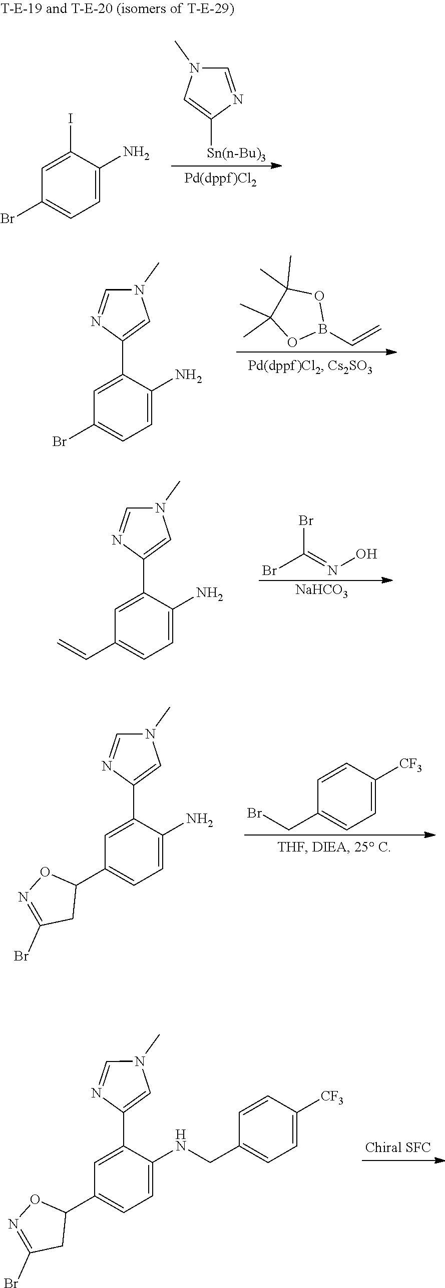

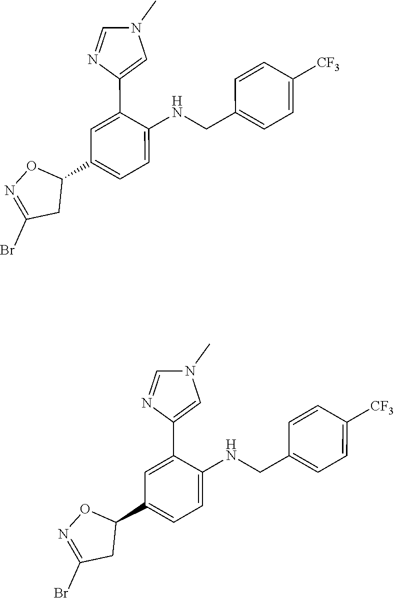 [SMILES: Cn1cnc(-c2cc([C@@H]3CC(Br)=NO3)ccc2NCc2ccc(C(F)(F)F)cc2)c1 ; Cn1cnc(-c2cc([C@H]3CC(Br)=NO3)ccc2NCc2ccc(C(F)(F)F)cc2)c1]

### Step 1: 4-Bromo-2-(1-methylimidazol-4-yl)aniline

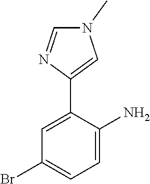

To a mixture of 4-bromo-2-iodo-aniline (2 g, 6.71 mmol, 1 eq) and tributyl-(1-methylimidazol-4-yl)stannane (2.72 g, 6.71 mmol, 91.5%, 1 eq) in DMF (20 mL) was added Pd(dppf)Cl₂ (491.22 mg, 671.32 μmol, 0.1 eq). The mixture was stirred under N₂ atmosphere at 130° C. for 12 h. The mixture was diluted with water (100 mL) and extracted with EtOAc (100 mL×3). The combined organic phase was washed with brine (50 mL), dried over anhydrous Na₂SO₄, filtered and concentrated in vacuum to yield a residue which was purified by flash silica gel chromatography (from PE/EtOAc=100/1 to 0/1, TLC: PE/EtOAc=0/1, R_{f}=0.30) to yield 4-bromo-2-(1-methylimidazol-4-yl)aniline (1 g, 3.25 mmol, 48.5% yield, 82.0% purity) as yellow oil. ¹H NMR (400 MHZ, CDCl₃) δ ppm 7.47-7.42 (m, 2H), 7.13-7.06 (m, 2H), 6.58 (d, J=8.6 Hz, 1H), 5.72-5.34 (m, 2H), 3.72 (s, 3H); ES-LCMS m/z 252.0, 254.0 [M+H]⁺.

### Step 2: 2-(1-Methylimidazol-4-yl)-4-vinyl-aniline

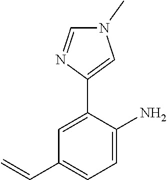

To a solution of 4-bromo-2-(1-methylimidazol-4-yl)aniline (1 g, 3.25 mmol, 82.0%, 1 eq) and 4,4,5,5-tetramethyl-2-vinyl-1,3,2-dioxaborolane (1.00 g, 6.51 mmol, 1.10 mL, 2 eq) in 1,4-dioxane (30 mL) and H₂O (6 mL) was added Pd(dppf)Cl₂ (237.99 mg, 325.25 μmol, 0.1 eq) and Cs₂CO₃ (3.18 g, 9.76 mmol, 3 eq). The mixture was stirred under N₂ atmosphere at 100° C. for 2 h. The mixture was diluted with water (100 mL) and extracted with EtOAc (100 mL×3). The combined organic phase was washed with brine (50 mL), dried over anhydrous Na₂SO₄, filtered and concentrated in vacuum to yield a residue which was purified by flash silica gel chromatography (from PE/EtOAc=100/1 to 0/1, TLC: PE/EtOAc=0/1, R_{f}=0.35) to yield 2-(1-methylimidazol-4-yl)-4-vinyl-aniline (500 mg, 2.16 mmol, 66.4% yield, 86.0% purity) as brown oil. ¹H NMR (400 MHZ, DMSO-d₆) δ ppm 7.68 (s, 1H), 7.56 (d, J=1.2 Hz, 1H), 7.46 (d, J=2.0 Hz, 1H), 7.06 (dd, J=2.0, 8.2 Hz, 1H), 6.62 (d, J=8.2 Hz, 1H), 6.55 (dd, J=11.0, 17.6 Hz, 1H), 6.43 (s, 2H), 5.53 (dd, J=1.2, 17.6 Hz, 1H), 4.94 (dd, J=1.0, 10.8 Hz, 1H), 3.70 (s, 3H); ES-LCMS m/z 200.3 [M+H]⁺.

### Step 3: 4-(3-Bromo-4,5-dihydroisoxazol-5-yl)-2-(1-methylimidazol-4-yl)aniline

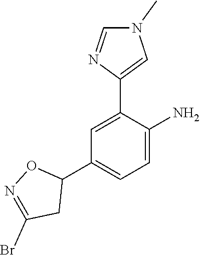 [SMILES: Cn1cnc(-c2cc(C3CC(Br)=NO3)ccc2N)c1]

To a solution of 2-(1-methylimidazol-4-yl)-4-vinyl-aniline (500 mg, 2.16 mmol, 86.0%, 1 eq) in EtOAc (10 mL) was added NaHCO₃ (1.81 g, 21.58 mmol, 839.35 μL, 10 eq) and dibromomethanone oxime (656.59 mg, 3.24 mmol, 1.5 eq). The mixture was stirred at 25° C. for 12 h. The reaction mixture was filtered and concentrated under reduced pressure to yield a residue which was purified by preparative TLC (PE/EtOAc=0/1, TLC: PE/EtOAc=0/1, R_{f}=0.20) to yield 4-(3-bromo-4,5-dihydroisoxazol-5-yl)-2-(1-methylimidazol-4-yl)aniline (150 mg, 434.35 μmol, 20.1% yield, 93.0% purity) as yellow oil. ¹H NMR (500 MHZ, CDCl₃) δ ppm 7.47 (s, 1H), 7.34 (d, J=1.8 Hz, 1H), 7.17 (s, 1H), 7.01-6.99 (m, 1H), 6.71 (d, J=8.2 Hz, 1H), 5.57 (t, J=10.2 Hz, 2H), 3.80-3.71 (m, 3H), 3.51 (dd, J=10.8, 17.3 Hz, 1H), 3.23 (dd, J=9.8, 17.3 Hz, 1H), 2.98-1.99 (m, 1H); ES-LCMS m/z 321.1, 323.1 [M+H]⁺.

### Step 4: 4-[(5S)-3-Bromo-4,5-dihydroisoxazol-5-yl]-2-(1-methylimidazol-4-yl)-N-[[4-(trifluoromethyl)phenyl]methyl]aniline and 4-[(5R)-3-bromo-4,5-dihydroisoxazol-5-yl]-2-(1-methylimidazol-4-yl)-N-[[4-(trifluoromethyl)phenyl]methyl]aniline

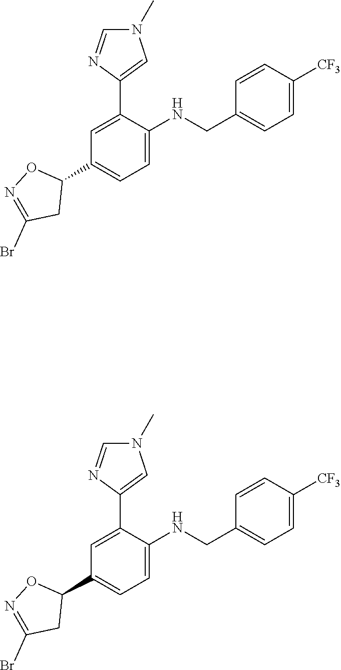 [SMILES: Cn1cnc(-c2cc([C@@H]3CC(Br)=NO3)ccc2NCc2ccc(C(F)(F)F)cc2)c1 ; Cn1cnc(-c2cc([C@H]3CC(Br)=NO3)ccc2NCc2ccc(C(F)(F)F)cc2)c1]

To a solution of 4-(3-bromo-4,5-dihydroisoxazol-5-yl)-2-(1-methylimidazol-4-yl)aniline (120 mg, 347.48 μmol, 93.0%, 1 eq) in THF (5 mL) was added DIEA (134.73 mg, 1.04 mmol, 181.57 μL, 3 eq) and 1-(bromomethyl)-4-(trifluoromethyl)benzene (166.12 mg, 694.95 μmol, 107.17 μL, 2 eq). The mixture was stirred at 25° C. for 12 h. The mixture was diluted with water (30 mL) and extracted with EtOAc (30 mL×3). The combined organic phase was washed with brine (20 mL), dried over anhydrous Na₂SO₄, filtered and concentrated in vacuum to yield a residue which was purified by preparative HPLC (column: Boston Prime C18 150*30 mm*5 μm; mobile phase: [water (0.05% NH₃·H₂O+10 mM NH₄HCO₃)-ACN]; B %: 60%-90%, 10 min), followed by lyophilization to yield a product. The product was separated by SFC (column: DAICEL CHIRALPAK IG (250 mm*50 mm, 10 μm); mobile phase: [0.1% NH₃·H₂O EtOH]; B %: 60%-60%) to yield peak 1 and peak 2. Peak 1 was concentrated under reduced pressure to yield a residue which was dissolved in MeCN (20 mL) and H₂O (40 mL) and lyophilized to yield 4-[(5S)-3-bromo-4,5-dihydroisoxazol-5-yl]-2-(1-methylimidazol-4-yl)-N-[[4-(trifluoromethyl)phenyl]methyl]aniline (24.61 mg, 50.72 μmol, 14.6% yield, 98.8% purity, SFC: Rₜ=2.248, ee=100%, [α]^{26.8}_{D}=+140.000 (MeOH, c=0.180 g/100 mL)) as a white solid. ¹H NMR (500 MHZ, CDCl₃) δ ppm 8.52 (s, 1H), 7.57 (d, J=8.1 Hz, 2H), 7.49 (d, J=10.2 Hz, 3H), 7.39 (d, J=2.0 Hz, 1H), 7.23 (s, 1H), 7.00 (dd, J=2.1, 8.5 Hz, 1H), 6.49 (d, J=8.4 Hz, 1H), 5.57 (t, J=10.2 Hz, 1H), 4.56 (s, 2H), 3.77 (s, 3H), 3.50 (dd, J=10.7, 17.4 Hz, 1H), 3.23 (dd, J=9.8, 17.3 Hz, 1H); ES-LCMS m/z 478.9, 480.9 [M+H]⁺. Peak 2 was concentrated under reduced pressure to yield a residue which was dissolved in MeCN (20 mL) and H₂O (40 mL) and lyophilized to yield 4-[(5R)-3-bromo-4,5-dihydroisoxazol-5-yl]-2-(1-methylimidazol-4-yl)-N-[[4-(trifluoromethyl)phenyl]methyl]aniline (24.58 mg, 50.61 μmol, 14.6% yield, 98.7% purity, SFC: Rₜ=3.301, ee=100%, [α]^{26.8}_{D}=−198.71 (MeOH, c=0.155 g/100 mL)) as a white solid. ¹H NMR (500 MHz, CDCl₃) δ ppm 8.54 (s, 1H), 7.58-7.55 (m, 2H), 7.49 (d, J=9.2 Hz, 3H), 7.39 (d, J=2.0 Hz, 1H), 7.23 (s, 1H), 7.00 (dd, J=2.0, 8.4 Hz, 1H), 6.49 (d, J=8.5 Hz, 1H), 5.57 (t, J=10.3 Hz, 1H), 4.56 (s, 2H), 3.77 (s, 3H), 3.50 (dd, J=10.8, 17.3 Hz, 1H), 3.23 (dd, J=9.8, 17.3 Hz, 1H); ES-LCMS m/z 478.9, 480.9 [M+H]⁺.

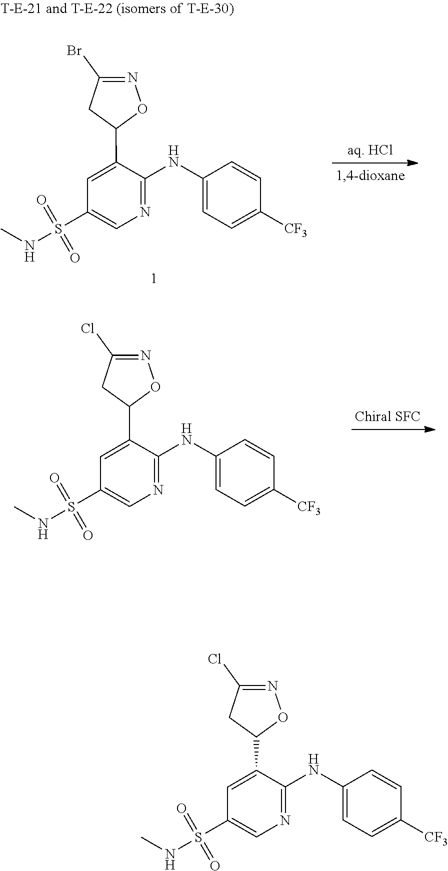

### Step 1: (S)-5-(3-Chloro-4,5-dihydroisoxazol-5-yl)-N-methyl-6-((4-(trifluoromethyl)phenyl)amino)pyridine-3-sulfonamide

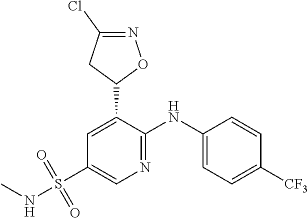

To a solution of 5-(3-bromo-4,5-dihydroisoxazol-5-yl)-N-methyl-6-[4-(trifluoromethyl)anilino]pyridine-3-sulfonamide (185 mg, 366.70 μmol, 95% purity, 1 eq) in 1,4-dioxane (10 mL) was added HCl (4 M, 0.5 mL). The mixture was stirred at 40° C. for 16 h. The solvent was removed and the residue was treated with water (10 mL), adjusted to pH 8 with sat. aq. NaHCO₃ and extracted with EtOAc (20 mL×2). The combined organic layers were washed with brine (20 mL), dried over Na₂SO₄, filtered and concentrated under reduced pressure to yield a residue which was purified by preparative TLC (PE/EtOAc=1/1, R_{f}=0.71) to yield a product which was separated by chiral SFC (column: DAICEL CHIRALPAK AD (250 mm*30 mm, 10 μm); mobile phase: [0.1% NH₃H₂O EtOH]; B %: 35%-35%) to yield Peak 1 and Peak 2. Peak 1 was concentrated under reduced pressure to yield a residue which was dissolved in MeCN (10 mL) and H₂O (20 mL) and lyophilized to yield (S)-5-(3-chloro-4,5-dihydroisoxazol-5-yl)-N-methyl-6-((4-(trifluoromethyl)phenyl)amino)pyridine-3-sulfonamide (43.65 mg, 97.09 μmol, 26.5% yield, 96.7% purity, SFC: Rₜ=1.279, ee=99.4%, [□]^{24.4}_{D}=−24.24 (MeOH, c=0.0825 g/100 mL) as a white solid. ¹H NMR (500 MHZ, CDCl₃) δ ppm 8.71 (d, J=2.4 Hz, 1H), 7.85 (d, J=2.3 Hz, 1H), 7.70-7.68 (m, 3H), 7.64-7.58 (m, 2H), 5.84 (t, J=11.2 Hz, 1H), 4.41 (br s, 1H), 3.62-3.41 (m, 2H), 2.71 (d, J=5.3 Hz, 3H); ES-LCMS m/z 435.0 [M+H]⁺.

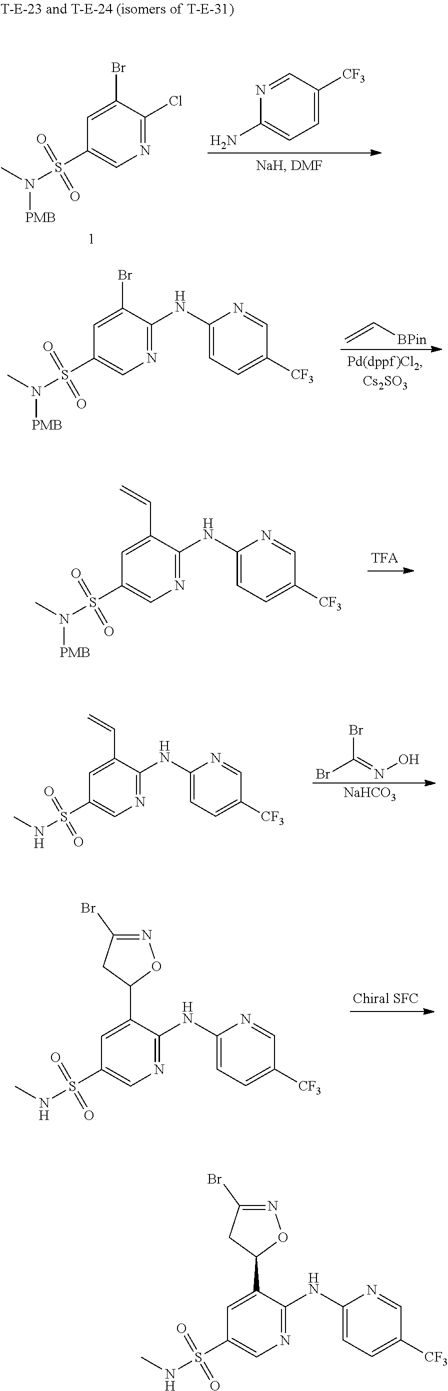

### Step 1: 5-Bromo-N-[(4-methoxyphenyl)methyl]-N-methyl-6-[[5-(trifluoromethyl)-2-pyridyl]amino]pyridine-3-sulfonamide

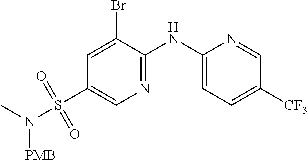

To a solution of 5-(trifluoromethyl)pyridin-2-amine (91.11 mg, 562.00 μmol, 1.5 eq) in DMF (3 mL) was added NaH (59.94 mg, 1.50 mmol, 60% purity, 4 eq) and the mixture was stirred at 0° C. for 0.5 h. 5-Bromo-6-chloro-N-[(4-methoxyphenyl)methyl]-N-methyl-pyridine-3-sulfonamide (160 mg, 374.67 μmol, 95% purity, 1 eq) was added and the mixture was stirred at 25° C. for 3 h. The reaction mixture was diluted with H₂O (20 mL) and extracted with EtOAc (40 mL×3). The combined organic phases were washed with brine (20 mL), dried over anhydrous Na₂SO₄, filtered and concentrated under reduced pressure to yield a residue. To the residue was added MeOH (5 mL) and the mixture was stirred at 25° C. for 2 h. The slurry was filtered and the cake was rinsed with MeOH (3 mL×2). The solid was collected and dried in vacuo to yield 5-bromo-N-[(4-methoxyphenyl)methyl]-N-methyl-6-[[5-(trifluoromethyl)-2-pyridyl]amino]pyridine-3-sulfonamide (160 mg, 301.12 μmol, 80.8% yield, 100.0% purity) as a white solid. ¹H NMR (400 MHZ, DMSO-d₆) δ ppm 9.21 (s, 1H), 8.72 (s, 1H), 8.68 (d, J=2.2 Hz, 1H), 8.41 (d, J=2.2 Hz, 1H), 8.28-8.19 (m, 2H), 7.24 (d, J=8.6 Hz, 2H), 6.93 (d, J=8.6 Hz, 2H), 4.16 (s, 2H), 3.74 (s, 3H), 2.60 (s, 3H); ES-LCMS m/z 533.0 [M+H]⁺.

### Step 2: N-[(4-Methoxyphenyl)methyl]-N-methyl-6-[[5-(trifluoromethyl)-2-pyridyl]amino]-5-vinyl-pyridine-3-sulfonamide

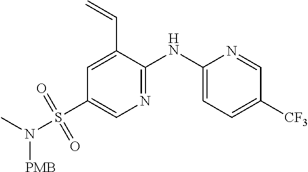

To a solution of 5-bromo-N-[(4-methoxyphenyl)methyl]-N-methyl-6-[[5-(trifluoromethyl)-2-pyridyl]amino]pyridine-3-sulfonamide (260 mg, 489.32 μmol, 100% purity, 1 eq) in 1,4-dioxane (6 mL) and H₂O (1 mL) was added 4,4,5,5-tetramethyl-2-vinyl-1,3,2-dioxaborolane (301.45 mg, 1.96 mmol, 331.99 μL, 4 eq), Pd(dppf)Cl₂ (35.80 mg, 48.93 μmol, 0.1 eq) and Cs₂CO₃ (318.86 mg, 978.64 μmol, 2 eq). The mixture was stirred under N₂ atmosphere at 90° C. for 12 h. The reaction mixture was diluted with H₂O (20 mL) and extracted with EtOAc (40 mL×3). The combined organic phases were washed with brine (20 mL), dried over anhydrous Na₂SO₄, filtered and concentrated under reduced pressure to yield a residue which was purified by flash silica gel chromatography (from PE/EtOAc=1/0 to 5/1, TLC: PE/EtOAc=5/1, R_{f}=0.35) to yield N-[(4-methoxyphenyl)methyl]-N-methyl-6-[[5-(trifluoromethyl)-2-pyridyl]amino]-5-vinyl-pyridine-3-sulfonamide (200 mg, 409.62 μmol, 83.7% yield, 98.0% purity) as a yellow solid. ¹H NMR (400 MHZ, CDCl₃) δ ppm 8.69 (d, J=2.3 Hz, 1H), 8.64 (d, J=8.8 Hz, 1H), 8.55 (s, 1H), 8.01-7.91 (m, 2H), 7.79 (s, 1H), 7.24 (d, J=8.8 Hz, 2H), 6.95-6.86 (m, 2H), 6.82 (dd, J=11.0, 17.3 Hz, 1H), 5.86 (d, J=17.3 Hz, 1H), 5.74 (d, J=11.0 Hz, 1H), 4.16 (s, 2H), 3.81 (s, 3H), 2.66 (s, 3H); ES-LCMS m/z 479.6 [M+H]⁺.

### Step 3: N-Methyl-6-[[5-(trifluoromethyl)-2-pyridyl]amino]-5-vinyl-pyridine-3-sulfonamide

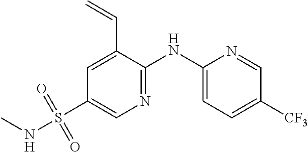 [SMILES: C=Cc1cc(S(=O)(=O)NC)cnc1Nc1ccc(C(F)(F)F)cn1]

To a solution of N-[(4-methoxyphenyl)methyl]-N-methyl-6-[[5-(trifluoromethyl)-2-pyridyl]amino]-5-vinyl-pyridine-3-sulfonamide (200 mg, 409.62 μmol, 98% purity, 1 eq) in DCM (3 mL) was added TFA (1.51 g, 13.24 mmol, 980.00 μL, 32.31 eq). The mixture was stirred at 25 ºC for 3 h. The solvent was removed to yield N-methyl-6-[[5-(trifluoromethyl)-2-pyridyl]amino]-5-vinyl-pyridine-3-sulfonamide (140 mg, crude) as a yellow solid. ¹H NMR (400 MHZ, CDCl₃) ¿ ppm 8.71 (d, J=2.0 Hz, 1H), 8.52 (s, 1H), 8.37 (d, J=8.8 Hz, 1H), 8.28 (s, 1H), 8.19 (d, J=7.1 Hz, 1H), 6.75 (s, 1H), 6.61 (s, 1H), 5.94 (d, J=16.9 Hz, 1H), 5.78 (d, J=11.0 Hz, 1H), 3.99 (s, 3H); ES-LCMS m/z 359.2 [M+H]⁺.

### Step 4: 5-[(5S)-3-Bromo-4,5-dihydroisoxazol-5-yl]-N-methyl-6-[[5-(trifluoromethyl)-2-pyridyl]amino]pyridine-3-sulfonamide and 5-[(5R)-3-bromo-4,5-dihydroisoxazol-5-yl]-N-methyl-6-[[5-(trifluoromethyl)-2-pyridyl]amino]pyridine-3-sulfonamide

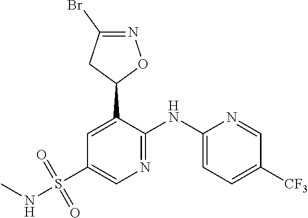 [SMILES: CNS(=O)(=O)c1cnc(Nc2ccc(C(F)(F)F)cn2)c([C@H]2CC(Br)=NO2)c1]

To a solution of N-methyl-6-[[5-(trifluoromethyl)-2-pyridyl]amino]-5-vinyl-pyridine-3-sulfonamide (140 mg, 390.69 μmol, 1 eq) in EtOAc (10 mL) was added NaHCO₃ (328.22 mg, 3.91 mmol, 151.95 μL, 10 eq) and dibromomethanone oxime (158.49 mg, 781.38 μmol, 2 eq). The mixture was stirred at 25° C. for 6 h. The mixture was filtered and the filtrate was concentrated to yield a residue which was purified by flash silica gel chromatography (from PE/EtOAc=1/0 to 1/1, TLC: PE/EtOAc=1/1, R_{f}=0.32) to yield a product which was separated by chiral SFC (column: DAICEL CHIRALPAK AD (250 mm*30 mm, 10 μm); mobile phase: [0.1% NH₃H₂O MeOH]; B %: 55%-55%) to yield Peak 1 and Peak 2. Peak 2 was concentrated under reduced pressure to yield 5-[(5R)-3-bromo-4,5-dihydroisoxazol-5-yl]-N-methyl-6-[[5-(trifluoromethyl)-2-pyridyl]amino]pyridine-3-sulfonamide (39.4 mg, 81.48 μmol, 20.9% yield, 99.3% purity, SFC: Rₜ=4.427, ee=99.9%, [α]^{24.5}_{D}=+39.22 (MeOH, c=0.051 g/100 mL) as a white solid. ¹H NMR (500 MHZ, DMSO-d₆) δ ppm 9.73 (s, 1H), 8.68-8.55 (m, 2H), 8.14-8.07 (m, 1H), 8.06-7.98 (m, 2H), 7.63 (q, J=4.8 Hz, 1H), 6.19 (dd, J=7.9, 11.0 Hz, 1H), 3.92 (dd, J=11.0, 17.5 Hz, 1H), 3.43-3.39 (m, 1H), 2.45 (d, J=4.9 Hz, 3H); ES-LCMS m/z 480.1 [M+H]⁺.

### Example 4. The Effects of a TEAD Inhibitor T-A-32, a MEK Inhibitor Trametinib, and a Combination Thereof on HCT-116 Tumor Growth in the HCT116 KRAS G13D Mutant Human Colorectal Carcinoma Xenograft Mouse Model

This study determined the in vivo antitumor activity of T-A-32 administered in combination with trametinib, a MEK inhibitor. This combination was tested in immunodeficient nude mice (Nu/Nu) bearing HCT-116 human colorectal carcinoma xenografts. HCT116 was chosen as a xenograft model because HCT116 harbors a the KRAS G13D mutation. The results demonstrated that T-A-32 in combination with trametinib has significant antitumor activity compared to vehicle control, and either agent alone, in female Nu/Nu mice bearing established HCT116 human colorectal carcinoma xenografts.

Six- to eight-week-old female Nu/Nu mice were inoculated subcutaneously with 1×10⁶ HCT116 human colorectal carcinoma tumor cells in the right flank. Tumor growth was monitored twice per week using vernier calipers and the mean tumor volume (MTV) was calculated. When the MTV reached approximately 150-200 mm³, approximately ten (10) days after cell inoculation, animals were randomized into treatment groups (n=10/group) and dosed orally (PO) with either vehicle control (5% DMSO+95% PEG 400 (Vehicle 1)+0.5% hydroxypropyl methyl cellulose and 0.2% Tween-80 (Vehicle 2)) or T-A-32 at 75 mg/kg, or trametinib at 0.5 mg/kg once per day (PO) for fourteen (14) days.

Tumor size and body weight were measured twice, and the study was terminated when the vehicle control tumors reached a mean of approximately 2000 mm³. Percent TGI was calculated on Day 14 when the control MTV reached the maximum allowable tumor volume. The mean maximum body weight change was determined for each group.

As shown herein, treatment with T-A-32 administered PO at 75 mg/kg QD (once a day) in combination with trametinib at 0.5 mg/kg resulted in significant antitumor activity compared with vehicle control (TGI=78%; p<0.0001). Accordingly, as shown in FIG. 3, the combination of T-A-32 and trametinib showed synergistic suppression of tumor growth.

### Example 5. The Effects of a TEAD Inhibitor T-A-32, a MEK Inhibitor Trametinib, and a Combination Thereof on A549 Tumor Growth in the A549 KRAS G12S Mutant Human Lunc Cancer Xenograft Mouse Model

This study determined the in vivo antitumor activity of T-A-32 administered in combination with trametinib, a MEK inhibitor. This combination was tested in immunodeficient nude mice (Nu/Nu) bearing A549 human lung cancer xenografts. A549 was chosen as a xenograft model because A549 cells harbor the KRAS G12S mutation. The results demonstrated that T-A-32 in combination with trametinib has significant antitumor activity compared to vehicle control, and either agent alone, in female Nu/Nu mice bearing established A549 human lung cancer xenografts.

Six- to eight-week-old female Nu/Nu mice were inoculated subcutaneously with 5×10⁶ A549 human lung cancer cells in the right flank. Tumor growth was monitored twice per week using vernier calipers and the mean tumor volume (MTV) was calculated. When the MTV reached approximately 150-200 mm³, approximately eight (8) days after cell inoculation, animals were randomized into treatment groups (n=8/group) and dosed orally (PO) with either vehicle control (5% DMSO+95% PEG 400 (Vehicle 1)+0.5% hydroxypropyl methyl cellulose and 0.2% Tween-80 (Vehicle 2)) or T-A-32 at 75 mg/kg, or trametinib at 1.0 mg/kg once per day (PO) for twenty-five (25) days.

Tumor size and body weight were measured twice per week, and all treatments ended on day 25. Percent TGI was calculated on Day 25 when the control MTV reached the maximum allowable tumor volume. The mean maximum body weight change was determined for each group.

As shown herein, treatment with T-A-32 administered PO at 75 mg/kg QD (once a day) in combination with trametinib at 1.0 mg/kg resulted in significant antitumor activity compared with vehicle control (TGI=83%; p<0.0001). Accordingly, as shown in FIG. 4, the combination of T-A-32 and trametinib showed synergistic suppression of tumor growth.

### Example 6. The Effects of a TEAD Inhibitor T-A-32, a MEK Inhibitor Trametinib, and a Combination Thereof on LoVo Tumor Growth in the LoVo KRAS G12D Mutant Human Colorectal Adenocarcinoma Xenograft Mouse Model

This study determined the in vivo antitumor activity of T-A-32 administered in combination with trametinib, a MEK inhibitor. This combination was tested in immunodeficient nude mice (Nu/Nu) bearing LoVo human colorectal adenocarcinoma xenografts. LoVo was chosen as a xenograft model because LoVo cells harbor the KRAS G12D mutation. The results demonstrated that T-A-32 in combination with trametinib has significant antitumor activity compared to vehicle control, and either agent alone, in female Nu/Nu mice bearing established LoVo human human colorectal adenocarcinoma xenografts.

Six- to eight-week-old female Nu/Nu mice were inoculated subcutaneously with 5×10⁶ LoVo human colorectal adenocarcinoma cells in the right flank. Tumor growth was monitored twice per week using vernier calipers and the mean tumor volume (MTV) was calculated. When the MTV reached approximately 150-200 mm³, approximately eight (8) days after cell inoculation, animals were randomized into treatment groups (n=10/group) and dosed orally (PO) with either vehicle control (5% DMSO+95% PEG 400 (Vehicle 1)+0.5% hydroxypropyl methyl cellulose and 0.2% Tween-80 (Vehicle 2)) or T-A-32 at 75 mg/kg, or trametinib at 1.0 mg/kg once per day (PO) for twenty-eight (28) days.

Tumor size and body weight were measured twice per week, and all treatments ended on day 28. Percent TGI was calculated on Day 28 when the control MTV reached the maximum allowable tumor volume. The mean maximum body weight change was determined for each group.

As shown herein, treatment with T-A-32 administered PO at 75 mg/kg QD (once a day) in combination with trametinib at 1.0 mg/kg resulted in significant antitumor activity compared with vehicle control (TGI=75%; p<0.0001). Accordingly, as shown in FIG. 5, the combination of T-A-32 and trametinib showed synergistic suppression of tumor growth.

While we have described a number of embodiments of this invention, it is apparent that our basic examples may be altered to provide other embodiments that utilize the compounds and methods of this invention. Therefore, it will be appreciated that the scope of this invention is to be defined by the application and claims rather than by the specific embodiments that have been represented by way of example.

1. A method of treating cancer in a patient in need thereof, comprising administering to the patient a therapeutically effective amount of a TEAD inhibitor and an EGFR inhibitor.

2. The method of claim 1, wherein the TEAD inhibitor is a compound of Formula A: 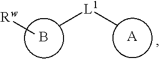
or a pharmaceutically acceptable salt thereof, wherein
L1 is C₁-6 bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—;
Ring A is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur;
Ring B is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, a 8-10 membered bicyclic heteroaromatic ring having 1-5 heteroatoms independently selected from nitrogen, oxygen, or sulfur;
R^{w} is an optionally substituted 4-, 5-, or 6-membered ring having 1, 2, 3, or 4 heteroatoms independently selected from nitrogen, oxygen, or sulfur; and
each R is independently —H or optionally substituted −C₁₋₆ aliphatic.

3. The method of claim 1, wherein the TEAD inhibitor is a compound of Formula B: 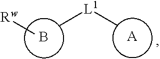
or a pharmaceutically acceptable salt thereof, wherein
L1 is a covalent bond, or a C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—;
Ring A is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur;
Ring B is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, a 8-10 membered bicyclic heteroaromatic ring having 1-5 heteroatoms independently selected from nitrogen, oxygen, or sulfur;
R^{w} is a warhead group; wherein when R^{w} is a saturated or partially unsaturated monocyclic carbocyclic or heterocyclic ring, it optionally forms a spiro bicyclic ring with Ring B; and
each R is independently —H or optionally substituted —C₁₋₆ aliphatic.

4. The method of claim 1, wherein the TEAD inhibitor is a compound of Formula C: 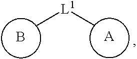
or a pharmaceutically acceptable salt thereof, wherein
L1 is a covalent bound, or a C₁₋₆ bivalent straight or branched hydrocarbon chain wherein 1, 2, or 3 methylene units of the chain are independently and optionally replaced with —N(R)—, —O—, or —C(O)—;
Ring A is selected from 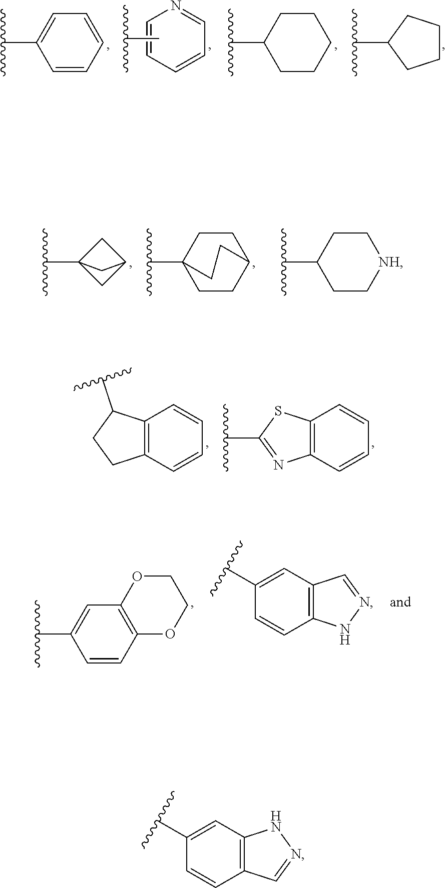
each of which is optionally substituted;
Ring B is selected from 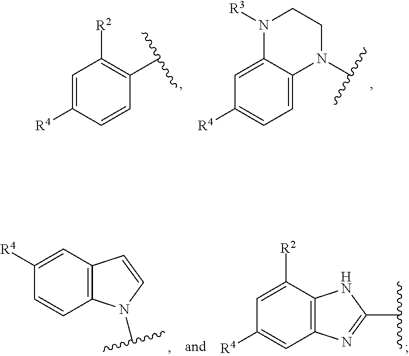
each R² is independently selected from —OR, —C(O)NR₂, optionally substituted —C₁₋₆ aliphatic, 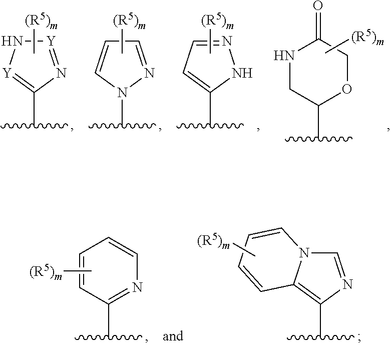
each Y is independently N or CR⁵;
R³ is H, —C(O)R, or optionally substituted —C₁₋₆ aliphatic;
each R⁴ is independently —S(O)₂NR₂, —S(O)₂R, —C(O)NR₂, —C(O)R, or optionally substituted —C₁₋₆ aliphatic;
each R⁵ is independently R, —CN, —C(O)R, —C(O)NR₂, or optionally substituted 5-6 membered heteroaryl having 1-2 heteroatoms independently selected from nitrogen, oxygen, or sulfur;
each m is independently 0, 1, or 2; and
each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

5. The method of claim 1, wherein the TEAD inhibitor is a compound of Formula D: 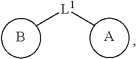
or a pharmaceutically acceptable salt thereof, wherein
L1 is a covalent bound, or a C₁₋₆ bivalent straight or branched hydrocarbon chain wherein 1, 2, or 3 methylene units of the chain are independently and optionally replaced with —N(R)—, —O—, or —C(O)—;
Ring A is selected from 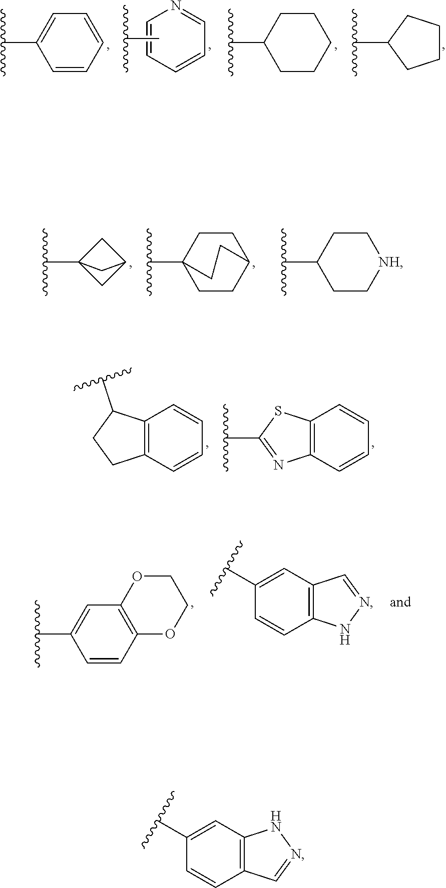
each of which is optionally substituted;
Ring B is 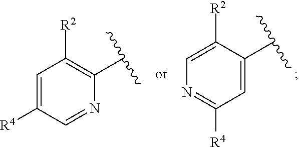
each R² is independently selected from —OR, —C(O)NR₂, optionally substituted —C₁₋₆ aliphatic, 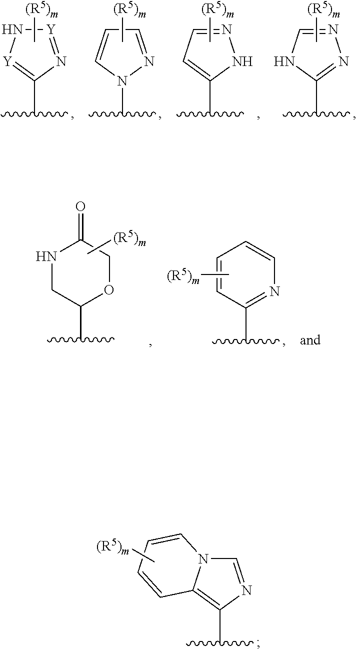
each Y is independently N or CR⁵;
each R⁴ is independently —S(O)₂NR₂, —S(O)₂R, —C(O)NR₂, —C(O)R, or optionally substituted —C₁₋₆ aliphatic;
each R⁵ is independently R, —CN, —C(O)R, —C(O)NR₂, or optionally substituted 5-6 membered heteroaryl having 1-2 heteroatoms independently selected from nitrogen, oxygen, or sulfur;
each m is independently 0, 1, or 2; and
each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

6. The method of claim 1, wherein the TEAD inhibitor is a compound of Formula E: 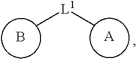
or a pharmaceutically acceptable salt thereof, wherein
L1 is a covalent bound, or a C₁₋₆ bivalent straight or branched hydrocarbon chain wherein 1, 2, or 3 methylene units of the chain are independently and optionally replaced with —N(R)—, —O—, or —C(O)—;
Ring A is selected from 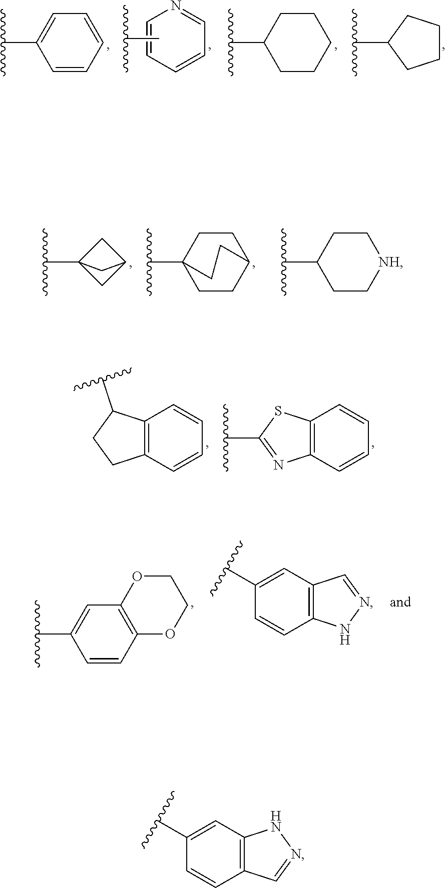
each of which is optionally substituted;
Ring B is selected from 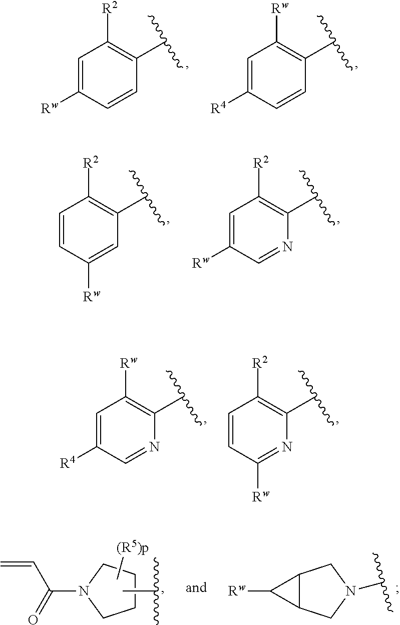
each R^{w} is independently selected from 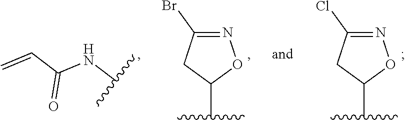
each R² is independently selected from —OR, —C(O)NR₂, optionally substituted —C₁₋₆ aliphatic, 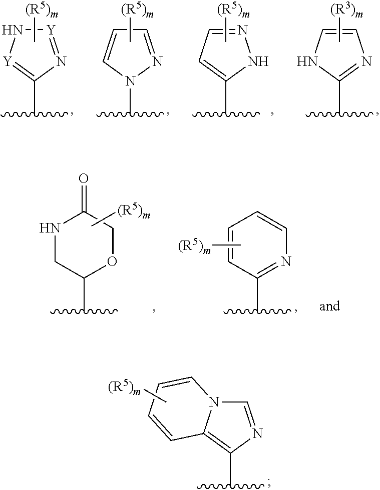
each Y is independently N or CR⁵;
each R³ is independently H or optionally substituted —C₁₋₆ aliphatic;
each R⁴ is independently —S(O)₂NR₂, —S(O)₂R, —C(O)NR₂, —C(O)R, or optionally substituted —C₁₋₆ aliphatic;
each R⁵ is independently R, —CN, —C(O)R, —C(O)NR₂, or optionally substituted 5-6 membered heteroaryl having 1-2 heteroatoms independently selected from nitrogen, oxygen, or sulfur;
each m is independently 0, 1, or 2;
p is 0, 1, or 2, and
each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

7. The method of claim 1, wherein the EGFR inhibitor is selected from cetuximab, necitumumab, panitumumab, zalutumumab, nimotuzumab, and matuzumab.

8. The method of claim 1, wherein the EGFR inhibitor is selected from osimertinib, gefitinib, erlotinib, lapatinib, neratinib, vandetanib, afatinib, brigatinib, dacomitinib, and icotinib.

9. The method of claim 1, further comprising administering an MEK inhibitor.

10. The method of claim 9, wherein the MEK inhibitor is selected from refametinib, selumetinib, trametinib, and cobimetinib.

11. The method of claim 1, wherein the cancer is an EGFR mutant resistant cancer.

12. The method of claim 11, wherein the cancer is an EGFR mutant resistant lung cancer, or an EGFR mutant resistant NSCLC.

13. A method of treating cancer in a patient in need thereof, comprising administering to the patient a therapeutically effective amount of a TEAD inhibitor and a MEK inhibitor.

14. The method of claim 13, wherein the TEAD inhibitor is a compound of Formula A: 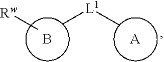
or a pharmaceutically acceptable salt thereof, wherein
L1 is C₁-6 bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—;
Ring A is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur;
Ring B is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, a 8-10 membered bicyclic heteroaromatic ring having 1-5 heteroatoms independently selected from nitrogen, oxygen, or sulfur;
R^{w} is an optionally substituted 4-, 5-, or 6-membered ring having 1, 2, 3, or 4 heteroatoms independently selected from nitrogen, oxygen, or sulfur; and
each R is independently —H or optionally substituted —C₁₋₆ aliphatic.

15. The method of claim 13, wherein the TEAD inhibitor is a compound of Formula B: 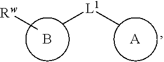
or a pharmaceutically acceptable salt thereof, wherein
L¹ is a covalent bond, or a C₁₋₆ bivalent straight or branched hydrocarbon chain wherein 1, 2, or 3 methylene units of the chain are independently and optionally replaced with —O—, —CH(OR)—, —CH(SR)—, —CH(N(R)₂)—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, or —(R)NC(S)N(R)—;
Ring A is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur;
Ring B is an optionally substituted ring selected from phenyl, a 4-, 5-, or 6-membered saturated or partially unsaturated monocyclic carbocyclic ring, a 4-, 5-, or 6-membered saturated or partially unsaturated monocyclic heterocyclic ring having 1-2 heteroatoms independently selected from nitrogen, oxygen, or sulfur, a 5-6 membered monocyclic heteroaromatic ring having 1, 2, 3, or 4 heteroatoms independently selected from nitrogen, oxygen, or sulfur, a 8-10 membered bicyclic aromatic ring, a 8-10 membered bicyclic heteroaromatic ring having 1-5 heteroatoms independently selected from nitrogen, oxygen, or sulfur;
R^{w} is a warhead group; wherein when R^{w} is a saturated or partially unsaturated monocyclic carbocyclic or heterocyclic ring, it optionally forms a spiro bicyclic ring with Ring B; and
each R is independently —H or optionally substituted —C₁₋₆ aliphatic.

16. The method of claim 13, wherein the TEAD inhibitor is a compound of Formula C: 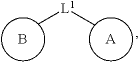
or a pharmaceutically acceptable salt thereof, wherein
L¹ is a covalent bound, or a C₁₋₆ bivalent straight or branched hydrocarbon chain wherein 1, 2, or 3 methylene units of the chain are independently and optionally replaced with —N(R)—, —O—, or —C(O)—;
Ring A is selected from 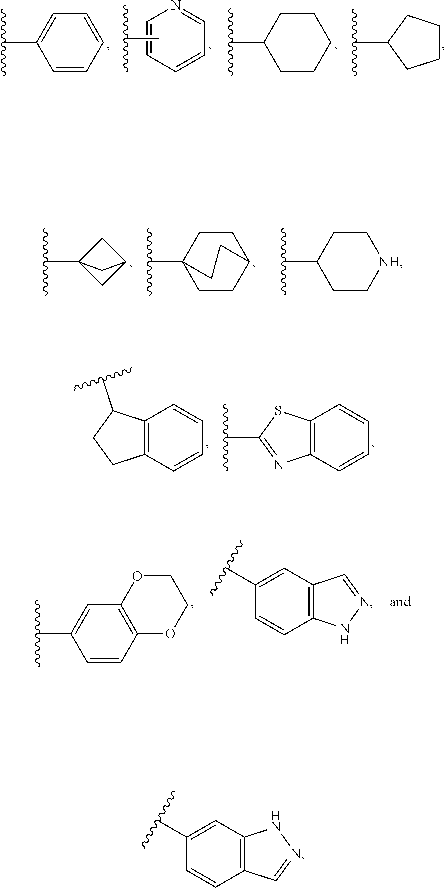
each of which is optionally substituted;
Ring B is selected from 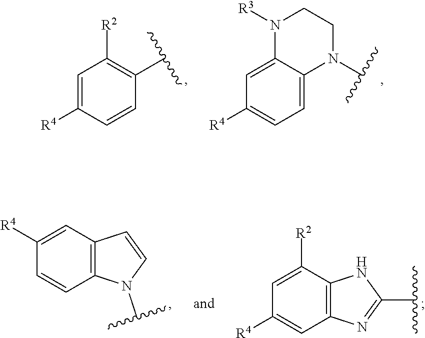
each R² is independently selected from —OR, —C(O)NR₂, optionally substituted —C₁₋₆ aliphatic, 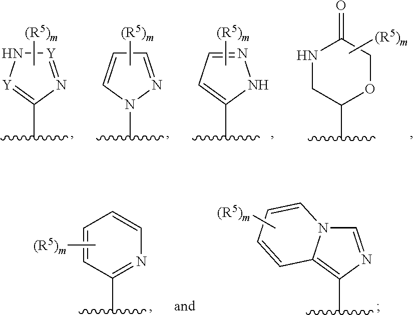
each Y is independently N or CR⁵;
R³ is H, —C(O)R, or optionally substituted —C₁₋₆ aliphatic;
each R⁴ is independently —S(O)₂NR₂, —S(O)₂R, —C(O)NR₂, —C(O)R, or optionally substituted —C₁₋₆ aliphatic;
each R⁵ is independently R, —CN, —C(O)R, —C(O)NR₂, or optionally substituted 5-6 membered heteroaryl having 1-2 heteroatoms independently selected from nitrogen, oxygen, or sulfur;
each m is independently 0, 1, or 2; and
each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

17. The method of claim 13, wherein the TEAD inhibitor is a compound of Formula D: 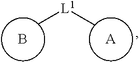
or a pharmaceutically acceptable salt thereof, wherein
L1 is a covalent bound, or a C₁₋₆ bivalent straight or branched hydrocarbon chain wherein 1, 2, or 3 methylene units of the chain are independently and optionally replaced with —N(R)—, —O—, or —C(O)—;
Ring A is selected from 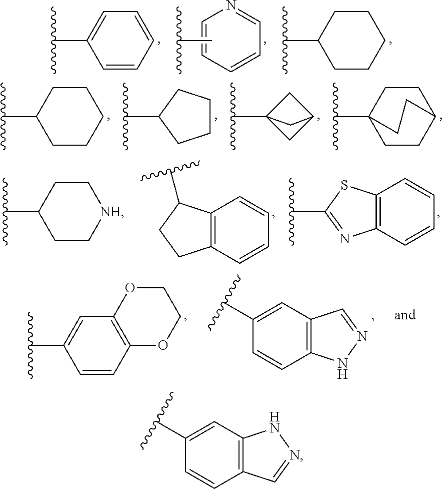
each of which is optionally substituted;
Ring B is 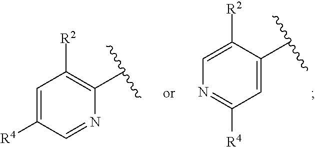
each R² is independently selected from —OR, —C(O)NR₂, optionally substituted —C₁₋₆ aliphatic, 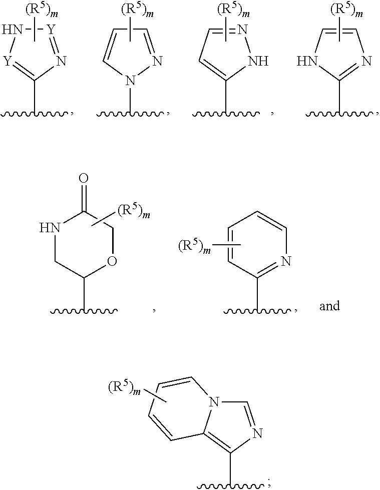
each Y is independently N or CR⁵;
each R⁴ is independently —S(O)₂NR₂, —S(O)₂R, —C(O)NR₂, —C(O)R, or optionally substituted —C₁₋₆ aliphatic;
each R⁵ is independently R, —CN, —C(O)R, —C(O)NR₂, or optionally substituted 5-6 membered heteroaryl having 1-2 heteroatoms independently selected from nitrogen, oxygen, or sulfur;
each m is independently 0, 1, or 2; and
each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

18. The method of claim 13, wherein the TEAD inhibitor is a compound of Formula E: 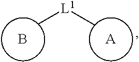
or a pharmaceutically acceptable salt thereof, wherein
L1 is a covalent bound, or a C₁₋₆ bivalent straight or branched hydrocarbon chain wherein 1, 2, or 3 methylene units of the chain are independently and optionally replaced with —N(R)—, —O—, or —C(O)—;
Ring A is selected from 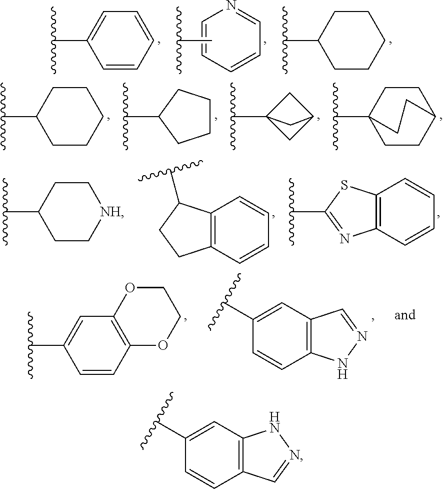
each of which is optionally substituted;
Ring B is selected from 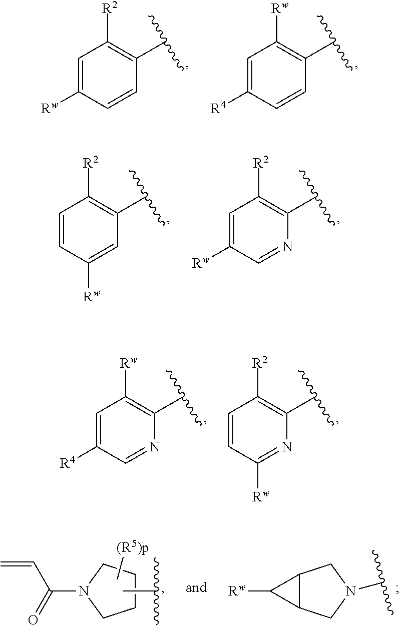
each R^{w} is independently selected from 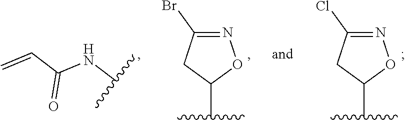
each R² is independently selected from —OR, —C(O)NR₂, optionally substituted —C₁₋₆ aliphatic, 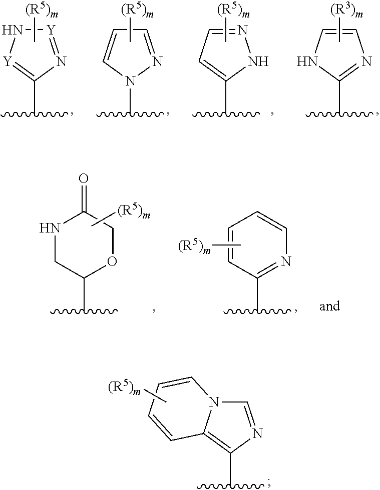
each Y is independently N or CR⁵;
each R³ is independently H or optionally substituted —C₁₋₆ aliphatic;
each R⁴ is independently —S(O)₂NR₂, —S(O)₂R, —C(O)NR₂, —C(O)R, or optionally substituted —C₁₋₆ aliphatic;
each R⁵ is independently R, —CN, —C(O)R, —C(O)NR₂, or optionally substituted 5-6 membered heteroaryl having 1-2 heteroatoms independently selected from nitrogen, oxygen, or sulfur;
each m is independently 0, 1, or 2;
p is 0, 1, or 2, and
each R is independently H, optionally substituted —C₁₋₆ aliphatic, optionally substituted 3-8 membered saturated or partially unsaturated monocyclic carbocyclyl, or optionally substituted 3-8 membered saturated or partially unsaturated monocyclic heterocyclyl having 1-2 heteroatoms independently selected from nitrogen, oxygen, or sulfur.

19. The method of claim 13, wherein the MEK inhibitor is selected from refametinib, selumetinib, trametinib, cobimetinib, binimetinib, mirdametinib, and pimasertib.

20. The method of claim 13, wherein the cancer is a KRAS mutant cancer that harbors one or more KRAS mutations selected from a KRAS G12C, a KRAS G12D mutation, a KRAS G12V mutation, and a KRAS G13 mutation.

21. (canceled)
